(19) United States
(12) Patent Application Publication
(10) Pub. No.: US 2024/0384332 A1
(43) Pub. Date: Nov. 21, 2024

(54) PROGRAMMABLE NUCLEASE DIAGNOSTIC DEVICE

(71) Applicant: Mammoth Biosciences, Inc., Brisbane, CA (US)

(72) Inventors: Jesus CHING, Saratoga, CA (US); James Paul BROUGHTON, South San Francisco, CA (US); Janice Sha CHEN, San Francisco, CA (US); Clare Louise FASCHING, Redwood City, CA (US); Phillip You Fai LEE, South San Francisco, CA (US); Brian J. EASTMAN, Napa, CA (US); Devin SPRATT, Mountain View, CA (US); Sarah Jane SHAPIRO, Pacifica, CA (US); Sonal JAIN, San Mateo, CA (US); Matthew VEROSLOFF, South San Francisco, CA (US); Jasmeet SINGH, Santa Clara, CA (US); Elizabeth M. HAWKINS, Redwood City, CA (US); Vaishnavi NAGESH, San Jose, CA (US); Lior KREINDLER, Foster City, CA (US); Sophia HUBBELL, San Francisco, CA (US); Carley Gelenter HENDRIKS, Burlingame, CA (US); Emma G. STANFIELD, San Francisco, CA (US); Amanda Claire BROWN, Brisbane, CA (US)

(21) Appl. No.: 18/393,023

(22) Filed: Dec. 21, 2023

Related U.S. Application Data
(63) Continuation of application No. PCT/US2022/034596, filed on Jun. 22, 2022.
(60) Provisional application No. 63/213,642, filed on Jun. 22, 2021.
(60) Provisional application No. 63/222,377, filed on Jul. 15, 2021.
(60) Provisional application No. 63/225,284, filed on Jul. 23, 2021.
(60) Provisional application No. 63/231,536, filed on Aug. 10, 2021.
(60) Provisional application No. 63/239,866, filed on Sep. 1, 2021.
(60) Provisional application No. 63/239,917, filed on Sep. 1, 2021.
(60) Provisional application No. 63/241,952, filed on Sep. 8, 2021.
(60) Provisional application No. 63/245,610, filed on Sep. 17, 2021.
(60) Provisional application No. 63/246,754, filed on Sep. 21, 2021.
(60) Provisional application No. 63/255,356, filed on Oct. 13, 2021.
(60) Provisional application No. 63/305,629, filed on Feb. 1, 2022.
(60) Provisional application No. 63/310,071, filed on Feb. 14, 2022.
(60) Provisional application No. 63/322,139, filed on Mar. 21, 2022.
(60) Provisional application No. 63/330,725, filed on Apr. 13, 2022.
(60) Provisional application No. 63/340,830, filed on May 11, 2022.
(60) Provisional application No. 63/351,751, filed on Jun. 13, 2022.

(51) Int. Cl. C12Q 1/6844; B01L 3/00; C12Q 1/34

(52) U.S. Cl. CPC C12Q 1/6844; B01L 3/5023; C12Q 1/34; B01L 2200/16; B01L 2300/0825; B01L 2400/06

(57) ABSTRACT

Various compositions and diagnostic devices for the detection of nucleic acids are provided. Also provided are systems comprising the same, and methods of using the same. In some embodiments, compositions and devices comprise or are configured to be used in combination with one or more of a guide nucleic acid, a programmable nuclease, and reporters. In some embodiments, presence of at least one sequence of interest is determined by detecting a signal produced upon cleavage of the reporters.

### CROSS-REFERENCE

This application claims priority to U.S. Provisional Application Ser. No. 63/213,642 filed on Jun. 22, 2021; U.S. Provisional Application Ser. No. 63/222,377 filed on Jul. 15, 2021; U.S. Provisional Application Ser. No. 63/225,284 filed on Jul. 23, 2021; U.S. Provisional Application Ser. No. 63/231,536 filed on Aug. 10, 2021; U.S. Provisional Application Ser. No. 63/239,866 filed on Sep. 1, 2021; U.S. Provisional Application Ser. No. 63/239,917 filed on Sep. 1, 2021; U.S. Provisional Application Ser. No. 63/241,952 filed on Sep. 8, 2021; U.S. Provisional Application Ser. No. 63/245,610 filed on Sep. 17, 2021; U.S. Provisional Application Ser. No. 63/246,754 filed on Sep. 21, 2021; U.S. Provisional Application Ser. No. 63/255,356 filed on Oct. 13, 2021; U.S. Provisional Application Ser. No. 63/305,629 filed on Feb. 1, 2022; U.S. Provisional Application Ser. No. 63/310,071 filed on Feb. 14, 2022; U.S. Provisional Application Ser. No. 63/322,139 filed on Mar. 21, 2022; U.S. Provisional Application Ser. No. 63/330,725 filed on Apr. 13, 2022; U.S. Provisional Application Ser. No. 63/340,830 filed on May 11, 2022; and U.S. Provisional Application Ser. No. 63/351,751 filed on Jun. 13, 2022, each of which is incorporated herein by reference in its entirety for all purposes.

### STATEMENT AS TO FEDERALLY SPONSORED RESEARCH

This invention was made with government support under Contract No. N66001-21-C-4048 awarded by the Department of Defense, Defense Advanced Research Projects Agency (DARPA). The US government has certain rights in the invention.

### SEQUENCE LISTING

The instant application contains a Sequence Listing which has been submitted electronically in XML format and is hereby incorporated by reference in its entirety. Said XML copy, created on Mar. 29, 2024, is named 203477-747301_US_SL.xml and is 2,182,193 bytes in size.

### BACKGROUND

Detection of ailments, especially at the early stages of disease or infection, can provide guidance on treatment or intervention to reduce the progression or transmission of said ailments. Such ailments can be detected at the point of need by devices capable of running diagnostic assays. Various biological species associated with an organism, disease state, phenotype, or genotype can be detected by these devices. Challenges in deploying such devices include developing methods to immobilize diagnostic assay components on surfaces without compromising the performance of the assay, as well as performing amplification of samples without significant additional instrumentation.

### SUMMARY

In an aspect, the present disclosure provides a programmable nuclease diagnostic device that may comprise a sample interface configured to receive a sample that may comprise at least one sequence of interest; a channel in fluid communication with the sample interface and a detection chamber, said channel comprising one or more movable mechanisms to separate the sample into a plurality of droplets, wherein said detection chamber is configured to receive and contact the plurality of droplets with at least one programmable nuclease probe disposed on a surface of said detection chamber, wherein said at least one programmable nuclease probe may comprise a guide nucleic acid complexed with a programmable nuclease; and a plurality of sensors that determine a presence of said at least one sequence of interest by detecting a signal produced upon cleavage of a target nucleic acid region of said at least one sequence of interest by said at least one programmable nuclease probe. In some embodiments, the cleavage of said target nucleic acid region occurs after a complementary binding of said target nucleic acid region to said guide nucleic acid of said at least one programmable nuclease probe. In some embodiments, the programmable nuclease probe may comprise a CRISPR/Cas enzyme. In some embodiments, the guide nucleic acid may comprise a guide RNA. In some embodiments, the one or more movable mechanisms comprise one or more valves configured to restrict flow through one or more sections of the channel. In some embodiments, the one or more movable mechanisms comprise a plunger or a bristle that is configured to restrict flow through one or more sections of the channel. In some embodiments, the one or more movable mechanisms are operatively coupled to a power source that is integrated with or insertable into the device. In some embodiments, the power source may comprise a battery. In some embodiments, the device may comprise a physical filter to filter one or more particles from the sample that do not comprise the sequence of interest. In some embodiments, the device may comprise a sample preparation chamber. In some embodiments, the sample preparation chamber may comprise a lysing agent. In some embodiments, the sample preparation chamber may comprise a heating unit configured for heat inactivation. In some embodiments, the sample preparation chamber may comprise one or more reagents for nucleic acid purification. In some embodiments, the channel may comprise a plurality of heating elements and a plurality of heat sinks for amplifying the at least one sequence of interest or a portion thereof. In some embodiments, the plurality of heating elements and the plurality of heat sinks are configured to perform one or more thermocycling operations on the plurality of droplets. In some embodiments, the device may comprise a plurality of programmable nuclease probes comprising different guide RNAs. In some embodiments, the signal is associated with a physical, chemical, or electrochemical change or reaction. In some embodiments, the signal may comprise an optical signal. In some embodiments, the signal may comprise a fluorescent or colorimetric signal. In some embodiments, the signal may comprise a potentiometric or amperometric signal. In some embodiments, the signal may comprise a piezo-electric signal. In some embodiments, the signal is associated with a change in an index of refraction of a solid or gel volume in which said at least one programmable nuclease probe is disposed.

In another aspect, the present disclosure provides a device that may comprise a sample interface configured to receive a sample that may comprise one or more target sequences of interest; one or more channels comprising one or more movable mechanisms to separate the sample into partitioned samples, wherein the one or more channels are in fluid communication with the sample interface and a reaction chamber that is configured to receive and contact the partitioned samples with an enzyme, reagent, or programmable detection agent that is configured to cleave a nucleic acid of said one or more target sequences of interest; and a plurality of sensors for determining a presence of the one or more target sequences of interest by detecting one or more reporters released upon said cleavage of said nucleic acid. In some embodiments, the programmable detection agent may comprise a CRISPR/Cas enzyme. In some embodiments, the one or more target sequences of interest comprise a sequence of nucleic acids comprising said nucleic acid. In some embodiments, the one or more movable mechanisms comprise a plurality of valves configured to restrict flow in a first direction through the one or more channels towards the sample interface. In some embodiments, the plurality of valves are configured to selectively permit flow in a second direction through the one or more channels towards the reaction chamber. In some embodiments, a first valve and a second valve of the plurality of valves are configured to physically, fluidically, or thermally isolate a first portion of the sample from a second portion of the sample when the first valve and the second valve are in a closed state. In some embodiments, the one or more channels comprise a plurality of heating elements and a plurality of heat sinks to perform thermocycling on the partitioned samples. In some embodiments, a first heating element of the plurality of heating elements and a first heat sink of the plurality of heat sinks are positioned between a first movable mechanism and a second movable mechanism of the one or more movable mechanisms. In some embodiments, the reporter may comprise a nucleic acid and a detection moiety. In some embodiments, the reporter may comprise at least one ribonucleotide or at least one deoxyribonucleotide. In some embodiments, the reporter may comprise a DNA nucleic acid or an RNA nucleic acid. In some embodiments, the device may comprise a telemedicine unit configured to provide one or more detection results to a computing unit that is remote from the device, wherein the one or more detection results indicate a presence or an absence of a target nucleic acid of interest in the sample.

In another aspect, the present disclosure provides a method for target detection, comprising: contacting a sample with any of the devices described herein; and detecting a presence or an absence of one or more genes of interest in said sample. In some embodiments, the method may comprise generating one or more detection results indicating the presence or the absence of the one or more genes of interest in the sample. In some embodiments, the method may comprise transmitting the one or more detection results to a remote computing unit. In some embodiments, the remote computing unit may comprise a mobile device. In another aspect, the present disclosure provides a method for target detection, comprising: providing a sample comprising at least one gene of interest; separating the sample into a plurality of sub-samples using one or more movable mechanisms; receiving the plurality of sub-samples in a detection chamber and contacting the plurality of sub-samples with at least one programmable nuclease probe disposed on a surface of said detection chamber, wherein said at least one programmable nuclease probe may comprise a guide nucleic acid complexed with a programmable nuclease; and using a plurality of sensors to determine a presence or an absence of said at least one gene of interest by detecting a signal produced upon cleavage of a target nucleic acid region in said at least one gene of interest by said at least one programmable nuclease probe. In some embodiments, the method may comprise amplifying the at least one gene of interest after separating the sample into a plurality of sub-samples. In some embodiments, the method may comprise amplifying the at least one gene of interest before the plurality of sub-samples are received in the detection chamber. In some embodiments, amplifying the at least one gene of interest may comprise using a plurality of heating elements and a plurality of heat sinks to perform thermocycling on the plurality of sub-samples. In some embodiments, the programmable nuclease probe may comprise a CRISPR/Cas enzyme. In some embodiments, the guide nucleic acid may comprise a guide RNA. In some embodiments, the one or more movable mechanisms comprise one or more valves configured to restrict flow through one or more sections of the channel. In some embodiments, the one or more movable mechanisms comprise a plunger or a bristle that is configured to restrict flow through one or more sections of the channel. In some embodiments, the method may comprise using a physical filter to filter one or more particles from the sample that do not comprise the at least one gene of interest. In some embodiments, the method may comprise lysing the sample before detecting the at least one gene of interest. In some embodiments, the method may comprise performing enzyme (e.g., PK) inactivation on the sample. In some embodiments, the method may comprise performing heat inactivation on the sample. In some embodiments, the method may comprise performing nucleic acid purification on the sample. In some embodiments, the method may comprise contacting the plurality of sub-samples with a plurality of programmable nuclease probes comprising different guide RNAs. In some embodiments, the signal is associated with a physical, chemical, or electrochemical change or reaction. In some embodiments, the signal is selected from the group consisting of an optical signal, a fluorescent signal, a colorimetric signal, a potentiometric signal, an amperometric signal, and a piezo-electric signal. In some embodiments, the signal is associated with a change in an index of refraction of a solid or gel volume in which said at least one programmable nuclease probe is disposed. In some embodiments, the method may comprise using the signal to detect pathogenic viruses, pathogenic bacteria, pathogenic worms, pathogenic fungi, or cancer cells. In some embodiments, the pathogenic viruses are selected from the group consisting of respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, and papillomavirus. In some embodiments, the method may comprise amplifying or modifying the signal using a physical or chemical interaction between a reporter that is released upon cleavage and another material, entity, or molecular species in the detection chamber. In some embodiments, the devices of the present disclosure are configured to detect pathogenic viruses, pathogenic bacteria, pathogenic worms, pathogenic fungi, or cancer cells based on the signal. In some embodiments, the pathogenic viruses are selected from the group consisting of respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, and papillomavirus. In some embodiments, the detection chamber or the reaction chamber of the device may comprise another material, entity, or molecular species that is configured to physically or chemically interact or react with a reporter that is released upon cleavage to amplify or modify the signal. In some embodiments, the sequence of interest may comprise a biological sequence. The biological sequence can comprise a nucleic acid sequence or an amino acid sequence. In some embodiments, the sequence of interest is associated with an organism of interest, a disease of interest, a disease state of interest, a phenotype of interest, a genotype of interest, or a gene of interest.

In another aspect, a reaction chamber may comprise a surface wherein a probe comprising at least one of said enzyme, said reagent, said programmable detection reagent, a programmable nuclease, a guide nucleic acid, a reporter or a combination thereof, and wherein said probe is immobilized to said surface by a linkage. In some embodiments, a linkage may comprise a surface functionality and a probe functionality. In some embodiments, a surface functionality is disposed on said surface. In some embodiments, a surface functionality is streptavidin. In some embodiments, an amino acid residue of said programmable nuclease is connected to said surface by said linkage. In some embodiments, an amino acid residue is modified with said probe functionality. In some embodiments, a probe functionality is biotin. In some embodiments, a guide nucleic acid is connected to said surface by said linkage. In some embodiments, a guide nucleic acid is modified at the 3′ end or 5′ end with said probe functionality. In some embodiments, a reporter is connected to said surface by said linkage. In some embodiments, a reporter may comprise at least one of said nucleic acid, said probe functionality, a detection moiety, a quencher or a combination thereof. In some embodiments, a reporter is configured for said detection moiety to remain immobilized to said surface and said quencher to be released into solution upon cleavage of said reporter. In some embodiments, a reporter is configured for said quencher to remain immobilized to said surface and for said detection moiety to be released into solution, upon cleavage of said reporter.

In one aspect, the present disclosure provides a composition comprising a plurality of reporters. In some embodiments, (a) each reporter of the plurality of reporters comprises one or more enzymes conjugated to a linker; (b) the linker comprises one or more nucleic acid sections; and (c) the linker (i) comprises a functionality for immobilization to a support, or (ii) is conjugated to the support. Conjugations can be covalent or non-covalent. In some embodiments, the one or more enzymes is a single enzyme, such as a single horseradish peroxidase (HRP). In some embodiments, the one or more enzymes comprises a poly-HRP comprising a plurality of HRP enzymes. In some embodiments, the plurality of HRP enzymes of the poly-HRP are complexed with streptavidin, and the linker comprises a biotin for conjugation with the poly-HRP. In some embodiments, the one or more nucleic acid sections comprise one or more sections of DNA, one or more sections of RNA, or a combination thereof. In some embodiments, the one or more nucleic acid sections consist of DNA. In some embodiments, the one or more nucleic acid sections consist of RNA. In some embodiments, the one or more nucleic acid sections comprise one or more sections of DNA and one or more sections of RNA. In some embodiments, the one or more nucleic acid sections are single-stranded. In some embodiments, the one or more nucleic acid sections have a combined length of at least 20, 30, 40, or 50 nucleotides. In some embodiments, at least one of the one or more nucleic acid sections is a substrate for cleavage by a programmable nuclease (e.g., any of the programmable nucleases described herein). In some embodiments, the linker comprises (a) a first portion comprising a non-naturally occurring guide nucleic acid, and (b) a second portion comprising a nucleic acid section that is a substrate for cleavage by a programmable nuclease, and the one or more enzymes (e.g., HRP) are conjugated to the linker via the second portion. In some embodiments, the linker further comprises one or more hydrocarbon chains. In some embodiments, the one or more hydrocarbon chains comprises a hydrocarbon chain between the one or more nucleic acid sections and the support. In some embodiments, the one or more hydrocarbon chains comprises a hydrocarbon chain between the one or more nucleic acid sections and the one or more enzymes. In some embodiments, the support is a bead, a hydrogel, or the surface of a container (e.g., a well or a tube). In some embodiments, the composition further comprises a programmable nuclease (e.g., any of the programmable nucleases described herein).

In one aspect, the present disclosure provides methods for detecting a target nucleic acid in a sample using a composition as described herein. In some embodiments, the method comprises (a) contacting the sample with a composition described herein to produce a reaction fluid, wherein (i) the linker is conjugated to the support, (ii) the composition further comprises a guide nucleic acid configured to bind to the target nucleic acid, and (iii) the programmable nuclease cleaves the linker in response to formation of a complex comprising the programmable nuclease, the guide nucleic acid, and the target nucleic acid; (b) separating the reaction fluid from the support; (c) contacting the separated reaction fluid with a substrate of the one or more enzymes, wherein presence of the one or more enzymes in the fluid produces a detectable signal from the enzyme substrate; and (d) detecting the detectable signal. In some embodiments, the method comprises (a) contacting the sample with a composition described herein to produce a reaction fluid, wherein (i) the composition further comprises a programmable nuclease and a guide nucleic acid, (ii) the guide nucleic acid is configured to bind to the target nucleic acid, and (iii) the programmable nuclease cleaves the linker in response to formation of a complex comprising the programmable nuclease, the guide nucleic acid, and the target nucleic acid; (b) contacting the reaction fluid with a plurality of beads, wherein each bead reacts with a plurality of the functionalities, thereby immobilizing the functionalities to the beads; (c) separating the reaction fluid from the beads; (d) contacting the separated reaction fluid with a substrate of the one or more enzymes, wherein presence of the one or more enzymes in the fluid produces a detectable signal from the enzyme substrate; and (e) detecting the detectable signal. In some embodiments, the method further comprises concentrating the one or more enzymes prior to contacting the one or more enzymes with the substrate, wherein the concentrating comprises separating the one or more enzymes from at least a portion of the separated reaction fluid. In some embodiments, concentrating comprises capture of the enzyme on a support. In some embodiments, contacting the separated reaction fluid with the substrate of the one or more enzymes comprises contacting the enzyme on the support with the substrate 2, 3, 4, 5, or more times. In some embodiments, the detectable signal comprises a change in an absorbance, and detection comprises detecting or measuring the change in absorbance.

In various aspects described herein, a reporter is connected to said surface by a linkage. In some embodiments, a linkage may comprise a surface functionality and a probe functionality. In some embodiments, a surface functionality is disposed on said surface and said reporter may comprise said probe functionality.

In certain aspects, described herein are embodiments of a method for target detection, comprising: providing a sample comprising at least one sequence of interest; separating the sample into a plurality of sub-samples using one or more movable mechanisms; receiving the plurality of sub-samples in a detection chamber and contacting the plurality of sub-samples with at least one probe, wherein said at least one probe is connected to a surface of said detection chamber by a linkage, wherein said at least one probe may comprise a programmable nuclease, a guide nucleic acid, a reporter or a combination thereof; and using a plurality of sensors to determine a presence of said at least one sequence of interest by detecting a signal produced upon cleavage of said reporter by said programmable nuclease.

Described herein are various embodiments of a device comprising: a sample interface configured to receive a sample that may comprise one or more target sequences of interest; one or more channels comprising one or more movable mechanisms to separate said sample into partitioned samples, wherein said one or more channels are in fluid communication with said sample interface and a reaction chamber comprising a surface, wherein at least one probe may comprise a programmable nuclease, a guide nucleic acid, a reporter or a combination thereof, wherein said at least one probe is connected to said surface by a linkage; and a plurality of sensors for determining a presence of said one or more target sequences of interest by detecting a signal emitted upon cleavage of said reporter by said programmable nuclease.

Described herein are various devices for detecting a target nucleic acid, comprising: a sample interface configured to receive a sample comprising a target nucleic acid; a reaction chamber (e.g., a heating region) in fluid communication with the sample interface and configured to amplify the sample received via the sample interface; a detection region in fluid communication with the heating region; and a programmable nuclease probe disposed within the sample interface, the heating region, and/or the detection region, wherein a signal is produced via selective binding between the programmable nuclease probe and the target nucleic acid within the heating region, the sample interface, and/or the detection region, wherein the detection region is configured to detect the signal corresponding to a presence of the target nucleic acid, and wherein the presence or absence of the target nucleic acid is determined within a time of less than 30 minutes after the sample is received at the sample interface. In some embodiments, a reagent mix comprising amplification reagents. In some embodiments, the reagent mix is lyophilized. In some embodiments, the reagent mix is located in a region of the device that is in fluid communication with both the sample interface and the heating region. In some embodiments, the reagent mix is located within the sample interface, the heating region, the detection region, and/or a region between the sample interface and the heating region. In some embodiments, the heating region comprises amplification reagents. In some embodiments, the sample is amplified via Loop-Mediated Isothermal Amplification (LAMP). In some embodiments, the heating region is configured to maintain an isothermal, or non-cycled temperature profile. In some embodiments, the isothermal, or non-cycled temperature profile is between about 30° C. to about 60° C. In some embodiments, the isothermal, or non-cycled temperature profile is about 55° C. to about 60° C. In some embodiments, the sample interface comprises a compartment configured to receive a swab containing the sample. In some embodiments, the compartment comprises a scraper configured to transfer the sample from the swab to the device. In some embodiments, the compartment contains a interface solution configured to extract the sample from the swab. In some embodiments, the interface solution comprises a buffer solution or a lysis buffer solution. In some embodiments, the sample interface is configured to receive the sample from a swab via pipetting. In some embodiments, the sample interface comprises a compartment configured to receive the sample from a container containing the sample. In some embodiments, the container comprises a syringe. In some embodiments, the syringe interface comprises an opening for receiving the sample therethrough. In some embodiments, the syringe interface opening is in fluid communication with i) the heating region, and/or ii) another compartment that is in fluid communication with the heating region. In some embodiments, the sample interface is configured to receive the sample as a fluid. In some embodiments, the heating region and detection region are disposed on the same location on the device. In some embodiments, the heating region and the detection region are disposed within a same compartment of the device. In some embodiments, the heating region comprises a channel for fluid movement therethrough. In some embodiments, the channel is in fluid communication with the sample interface and the detection region, either directly or indirectly, thereby enabling the sample to move from the sample interface to the detection region. In some embodiments, the channel comprises a spiral configuration or a serpentine configuration. In some embodiments, two or more channels for fluid movement therethrough, wherein at least one channel of the two or more channels is configured to move the sample from the sample interface to the detection region. In some embodiments, each channel of the heating region comprises one or more movable mechanisms. In some embodiments, the one or more movable mechanisms comprises i) a first movable mechanism between the sample interface and heating region for controlling transfer of the sample therebetween, and/or ii) a second movable mechanism between the heating region and the detecting region for controlling transfer of the sample therebetween. In some embodiments, at least one channel of the heating region comprises two or more heating compartments configured to separate the sample into two or more sub-samples, wherein the two or more compartments are separated from each other via a movable mechanism of the one or more movable mechanisms. In some embodiments, each heating compartment is configured to be heated by a corresponding heating element. In some embodiments, at least one heating element of the device comprises a chemical heating element. In some embodiments, at least one chemical heating element is sodium acetate. In some embodiments, the heating region comprises a chamber in fluid communication with the sample interface and the detection region. In some embodiments, the heating region comprises a reporter immobilized therein, wherein the report is configured to release a detection moiety via the selective binding between the activated programmable nuclease and the target nucleic acid, thereby enabling the signal to be produced. In some embodiments, the reporter is immobilized in the heating region via a support that is immobilized on a surface of the heating region. In some embodiments, the support comprises a bead, a coating, and an interspersed polymer. In some embodiments, the support comprises a solid support. In some embodiments, the surface of the heating region comprises a well that is a recessed portion of the surface, wherein the support is disposed within the well. In some embodiments, one or more channels are configured to move the sample from the sample interface to the detection region. In some embodiments, the one or more channels are located within the sample interface, between the sample interface and the heating region, within the heating region, between the heating region and the detection region, and/or within the detection region. In some embodiments, one or more channels comprises a plurality of channels, wherein the plurality of channels comprises at least one set of channels arranged in series. In some embodiments, the one or more channels comprises a plurality of channels, wherein the plurality of channels comprises at least one set of channels arranged in parallel (parallel channels), thereby enabling the sample to be split into sub-samples within each channel of the at least one set of parallel channels. In some embodiments, the one or more channels comprises a plurality of channels, wherein the plurality of channels comprises at least one set of channels configured to move the sample from a first location within the device to a second location within the device, thereby enabling the sample to be split into sub-samples within each channel of the at least one set of channels In some embodiments, the at least one set of channels comprises two or more channels having a different length and/or different configuration, thereby enabling specific conditions to be specified for two or more corresponding sub-samples. In some embodiments, the specific conditions comprise a specified heating temperature range, a specified heating duration, a specified residence time within any region or location on the device, a specific incubation time, contact with specific reagents, or any combination thereof. In some embodiments, the at least one set of channels comprises two or more channels having a same length and/or configuration, thereby enabling specific conditions to be specified for two or more corresponding sub-samples. In some embodiments, a channel of the one or more channels comprises a radial configuration, a spiral configuration, a serpentine configuration, a linear configuration, or any combination thereof. In some embodiments, at least one actuator. In some embodiments, the at least one actuator comprises a plunger, a spring-actuated plunger, or a spring mechanism. In some embodiments, the at least one actuator is manually actuated. In some embodiments, a first actuator of the at least one actuator is configured to move the sample from the sample interface to the heating region via manual actuation of the first actuator. In some embodiments, a second actuator of the at least one actuator is configured to move the detection moiety from the heating region to the detection region via manual actuation of the second actuator. In some embodiments, the device is configured to be operated manually without electrical power. In some embodiments, a power source. In some embodiments, the power source comprises one or more batteries. In some embodiments, the heating region is configured to heat the sample via a heating element. In some embodiments, the heating element comprises a chemical heating element. In some embodiments, the chemical heating element is sodium acetate. In some embodiments, the signal is visually detectable. In some embodiments, the programmable nuclease comprises a guide nucleic acid. In some embodiments, the guide nucleic acid is modified. In some embodiments, the guide nucleic acid is modified with at least one methyl group. In some embodiments, the programmable nuclease further comprises a Cas enzyme. In some embodiments, the Cas enzyme is selected from the group consisting Cas12, Cas13, Cas14, Cas14a, Cas14a1, and CasPhi. In some embodiments, the target nucleic acid is indicative of a respiratory disorder or respiratory pathogen. In some embodiments, the respiratory disorder or respiratory pathogen selected from the group consisting of SARS-CoV-2 and corresponding variants, 29E), NL63, OC43, HKU1, MERS-CoV, (MERS), SARS-CoV (SARS, Flu A, Flu B, RSV, Rhinovirus, Strep A, and TB. In some embodiments, the device is configured to differentiate between a viral infection and a bacterial infection. In some embodiments, the target nucleic acid is indicative of a sexually transmitted infection (STI) or infection related to a woman's health. In some embodiments, the STI or infection related to a woman's health is selected from the group consisting of CT, NG, MG, TV, HPV, Candida, B. Vaginosis Syphilis and UTI. In some embodiments, the target nucleic acid comprises a single nucleotide polymorphism (SNP). In some embodiments, the SNP is indicative of NASH disorder or Alpha-1 disorder. In some embodiments, the target nucleic acid is a blood borne pathogen selected from the group consisted of HIV, HBV, HCV and Zika. In some embodiments, the target nucleic acid is indicative of H. Pylori, C. Difficile, Norovirus, HSV and Meningitis. In some embodiments, a physical filter configured to filter one or more particles from the sample that do not comprise the target nucleic acid. In some embodiments, the physical filter is located between and in fluid communication with the sample interface and heating region. In some embodiments, the programmable nuclease, guide nucleic acid, or the reporter are immobilized to a device surface by a linkage. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface. In some embodiments, a plurality of guide nucleic acids, wherein each guide nucleic acid of the plurality of guide nucleic acids is complementary, or partially complementary to a different segment of the target nucleic acid. In some embodiments, the sample comprises the sample containing the target nucleic acid(s), the sample containing the amplification reagents, the amplified sample, and/or the sample containing the detection moiety.

Described herein are various devices for detecting a target nucleic acid in a sample, comprising: a sample interface for receiving the sample; a reaction chamber (e.g., a heating region) in fluid communication with the sample interface, the heating region comprising: a programmable nuclease comprising a guide nucleic acid, and a reporter, wherein the programmable nuclease is activated by selective binding between the guide nucleic acid and a target nucleic acid, wherein the reporter is configured to release a detection moiety upon cleavage by the activated programmable nuclease; a chemical heating element configured to heat to the heating region; a detection region in fluid communication with the heating region, wherein the detection region is configured to detect a signal produced by the released detection moiety; a first manual actuator configured to transfer the sample from the heating region to the detection region; and a reagent mix comprising amplification reagents, wherein the reagent mix is disposed within the sample interface, the heating region, the detection region, and/or between the sample interface and the heating region, wherein the device is configured to determine the presence or absence of the target nucleic acid within a time of less than 30 minutes via the produced signal. In some embodiments, the reagent mix is lyophilized. In some embodiments, the heating region is configured to amplify the target nucleic acid. In some embodiments, the heating region comprises the amplification reagents. In some embodiments, the target nucleic acid is amplified via Loop-Mediated Isothermal Amplification (LAMP). In some embodiments, the heating region is configured to maintain an isothermal, or non-cycled temperature profile. In some embodiments, the isothermal, or non-cycled temperature profile is between about 30° C. to about 60° C. In some embodiments, the isothermal, or non-cycled temperature profile is about 55° C. to about 60° C. In some embodiments, the sample interface comprises a compartment configured to receive a swab containing the sample. In some embodiments, the compartment comprises a scraper configured to transfer the sample from the swab to the device. In some embodiments, the compartment contains an interface solution configured to extract the sample from the swab. In some embodiments, the interface solution comprises a buffer solution or a lysis buffer solution. In some embodiments, the sample interface is configured to receive the sample from a swab via pipetting. In some embodiments, the sample interface comprises a compartment configured to receive the sample from a container containing the sample. In some embodiments, the container comprises a syringe. In some embodiments, the syringe interface comprises an opening for receiving the sample therethrough. In some embodiments, the syringe interface opening is in fluid communication with the heating region, or another compartment that is in fluid communication with the heating region. In some embodiments, the sample interface is configured to receive the sample as a fluid. In some embodiments, the heating region and detection region are disposed on the same location on the device. In some embodiments, the heating region and the detection region are disposed within a same compartment of the device. In some embodiments, the heating region comprises a channel for fluid movement therethrough. In some embodiments, the channel is in fluid communication with the sample interface and the detection region, either directly or indirectly, thereby enabling the sample to move from the sample interface to the detection region. In some embodiments, the channel comprises a spiral configuration or a serpentine configuration. In some embodiments, two or more channels for fluid movement therethrough, wherein at least one channel of the two or more channels is configured to move the sample from the sample interface to the detection region. In some embodiments, each channel of the heating region comprises one or more movable mechanisms. In some embodiments, the one or more movable mechanisms comprises i) a first movable mechanism between the sample interface and heating region for controlling transfer of the sample therebetween, and/or ii) a second movable mechanism between the heating region and the detecting region for controlling transfer of the sample therebetween. In some embodiments, at least one channel of the heating region comprises two or more heating compartments configured to separate the sample into two or more sub-samples, wherein the two or more compartments are separated from each other via a movable mechanism of the one or more movable mechanism. In some embodiments, each heating compartment is configured to be heated by a corresponding heating element of at least one heating element. In some embodiments, the at least one heating element of the device comprises a chemical heating element. In some embodiments, the at least one chemical heating element is sodium acetate. In some embodiments, the heating region comprises a chamber. In some embodiments, the heating region comprises the reporter immobilized therein. In some embodiments, the reporter is immobilized in the heating region via a support that is immobilized on a surface of the heating region. In some embodiments, the support comprises a bead, a coating, and an interspersed polymer. In some embodiments, the support comprises a solid support. In some embodiments, the surface of the heating region comprises a well that is recessed portion of the surface, wherein the support is disposed within the well. In some embodiments, one or more channels are configured to move the sample from the sample interface to the detection region. In some embodiments, the one or more channels are located within the sample interface, between the sample interface and the heating region, within the heating region, between the heating region and the detection region, and/or within the detection region. In some embodiments, the one or more channels comprises a plurality of channels, wherein the plurality of channels comprises at least one set of channels arranged in series. In some embodiments, the one or more channels comprises a plurality of channels, wherein the plurality of channels comprises at least one set of parallel channels arranged in parallel (parallel channels), thereby enabling the sample to be split into sub-samples within each channel of the at least one set of parallel channels. In some embodiments, the one or more channels comprises a plurality of channels, wherein the plurality of channels comprises at least one set of channels configured to move the sample from a first location within the device to a second location within the device, thereby enabling the sample to be split into sub-samples within each channel of the at least one set of channels. In some embodiments, the at least one set of channels comprises two or more channels having a different length and/or different configuration, thereby enabling specific conditions to be specified for two or more corresponding sub-samples. In some embodiments, the specific conditions comprise a specified heating temperature range, a specified heating duration, a specified residence time within any region or location on the device, a specific incubation time, contact with specific reagents, or any combination thereof. In some embodiments, the at least one set of channels comprises two or more channels having a same length and/or configuration, thereby enabling specific conditions to be specified for two or more corresponding sub-samples. In some embodiments, a channel of the one or more channels comprises a radial configuration, a spiral configuration, a serpentine configuration, a linear configuration, or any combination thereof. In some embodiments, the at least one actuator comprises a plunger, a spring-actuated plunger, or a spring mechanism. In some embodiments, the device is configured to be operated manually without electrical power. In some embodiments, the device may comprise a power source. In some embodiments, the power source comprises one or more batteries. In some embodiments, the heating region is configured to heat the sample via a heating element. In some embodiments, the heating element comprises a chemical heating element. In some embodiments, the chemical heating element is sodium acetate. In some embodiments, the signal is visually detectable. In some embodiments, the guide nucleic acid is modified. In some embodiments, the guide nucleic acid is modified with at least one methyl group. In some embodiments, the programmable nuclease further comprises a Cas enzyme. In some embodiments, the Cas enzyme is selected from the group consisting Cas12, Cas13, Cas14, Cas14a, Cas14a1, and CasPhi. In some embodiments, the target nucleic acid is indicative of a respiratory disorder or respiratory pathogen. In some embodiments, the respiratory disorder or respiratory pathogen selected from the group consisting of SARS-CoV-2 and corresponding variants, 29E), NL63, OC43, HKU1, MERS-CoV, (MERS), SARS-CoV (SARS, Flu A, Flu B, RSV, Rhinovirus, Strep A, and TB. In some embodiments, the device is configured to differentiate between a viral infection and a bacterial infection. In some embodiments, the target nucleic acid is indicative of a sexually transmitted infection (STI) or infection related to a woman's health. In some embodiments, the STI or infection related to a woman's health is selected from the group consisting of CT, NG, MG, TV, HPV, Candida, B. Vaginosis Syphilis, and UTI. In some embodiments, the target nucleic acid comprises a single nucleotide polymorphism (SNP). In some embodiments, the SNP is indicative of NASH disorder or Alpha-1 disorder. In some embodiments, the target nucleic acid is a blood borne pathogen selected from the group consisted of HIV, HBV, HCV, and Zika. In some embodiments, the target nucleic acid is indicative of H. Pylori, C. Difficile, Norovirus, HSV, and Meningitis. In some embodiments, a physical filter configured to filter one or more particles from the sample that do not comprise the target nucleic acid. In some embodiments, the physical filter is located between and in fluid communication with the sample interface and heating region. In some embodiments, the programmable nuclease, guide nucleic acid, or the reporter are immobilized to a device surface by a linkage. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface. In some embodiments, a plurality of guide nucleic acids, wherein each guide nucleic acid of the plurality of guide nucleic acids is complementary, or partially complementary to a different segment of the target nucleic acid. In some embodiments, the sample comprises the sample containing the target nucleic acid(s), the sample containing the amplification reagents, the amplified sample, and/or the sample containing the detection moiety.

Described herein are various embodiments for a microarray device for multiplexed detection of a plurality of target nucleic acids in a sample, comprising: a sample interface for receiving the sample; a reagent mix comprising amplification reagents; a reaction chamber (e.g., a heating region) in fluid communication with the sample interface; and a detection region comprising a surface comprising a plurality of detection spots in a microarray format, wherein each of the plurality of detection spots comprise a reporter probe, wherein each reporter probe is configured to release a detection moiety via cleavage by an activated programmable nuclease, wherein each of the plurality of detection spots comprise a different programmable nuclease probe, and wherein the device is configured to determine the presence or absence of each of the plurality of target nucleic acids within a time of less than 30 minutes via the release of each detection moiety of each reporter at each of the plurality of detection spots. In some embodiments, at least one programmable nuclease probe comprises a Cas enzyme. In some embodiments, each Cas enzyme of the at least one programmable nuclease is selected from the group comprising of Cas12, Cas13, Cas14, Cas14a, and Cas14a1. In some embodiments, the microarray device may comprise all the various embodiments of devices as described herein.

Described herein are various embodiment of a kit for the detection of a target nucleic acid in a sample, the kit comprising: a swab; elution reagents; lysis reagents; a device comprising a sample interface for receiving the sample; a reaction chamber (e.g., a heating region) in fluidic communication with the sample interface and configured to receive the sample, the heating region comprising a programmable nuclease comprising a guide nucleic acid and a reporter disposed within the heating region, wherein the programmable nuclease is activated by selective binding between the guide nucleic acid and the target nucleic acid, wherein the reporter is configured to release a detection moiety via the activated programmable nuclease; a chemical heating element configured to provide heat to the heating region; a detection region in fluid communication with the heating region and the sample interface, wherein the detection region is configured to detect a signal produced by the released detection moiety, thereby detecting the presence of the target nucleic acid; and a reagent mix comprising amplification reagents, wherein the reagent mix is disposed within the sample interface, the heating region, the detection region, and/or between the sample interface and the heating region, and wherein the device is configured to determine the presence or absence of the target nucleic acid within a time of less than 30 minutes via the released detection moiety. In some embodiments, the sample interface is configured to receive the sample from the swab. In some embodiments, a collection tube, wherein the collection tube is configured to accept the swab, wherein the sample contained in the swab is transferred to the collection tube, and wherein the collection tube is separate from the device. In some embodiments, the collection tube is configured to be inserted into the sample interface to transfer the sample to the device. In some embodiments, the collection tube is a syringe. In some embodiments, the kit comprises a first container containing the elution reagents and/or the lysis reagents. In some embodiments, the kit comprises dilution reagents. In some embodiments, the kit comprises a second container containing the dilution reagents. In some embodiments, the programmable nuclease comprises a Cas enzyme. In some embodiments, the Cas enzyme is selected from the group comprising of Cas12, Cas13, Cas14, Cas14a, and Cas14a1. In some embodiments, the kit may comprise all the various embodiments of devices, as described herein.

Described herein are various embodiments of a method for the detection of a target nucleic acid in a sample, the method comprising: providing a device configured to determine a presence or absence of a target nucleic acid in less than 30 minutes after a sample is introduced into the device, the device comprising a sample interface, a heating region in fluid communication with the sample interface, and a detection region in fluid communication with the heating region; introducing the sample into the sample interface of the device; mixing the sample with a reagent mix comprising amplification reagents to generate a mixed sample solution; transferring the mixed sample solution from the sample interface to the heating region; amplifying the sample by heating the mixed sample solution in the heating region; performing a programmable nuclease-based assay, wherein selective binding between a guide nucleic acid and the target nucleic acid activates a programmable nuclease probe configured to cleave a reporter probe, thereby releasing a detection moiety into the sample solution when the target nucleic acid is present; transferring the mixed sample solution with the amplified sample from the heating region to the detection region; and determining the presence or absence of the target nucleic acid in the sample via capture of the released detection moiety in the detection region. In some embodiments, the amplifying the target nucleic acid and the performing the programmable nuclease assay are performed as a one-pot reaction in the heating region. In some embodiments, the one-pot reaction is performed between about 30° C. to 60° C. In some embodiments, the one-pot reaction is performed at about 55° C. In some embodiments, the programmable nuclease of the one-pot reaction comprises a Cas enzyme, the Cas enzyme selected from the group comprising of Cas12, Cas13, Cas14, Cas14a, and Cas14a1. In some embodiments, the method further comprises filtering the sample with the reagent mix prior to entering the heating region. In some embodiments, the method further comprises filtering the sample comprises filtering one or more particles from the sample that do not comprise the target nucleic acid. In some embodiments, the filter is located between and in fluid communication with the sample interface and heating region. In some embodiments, the method further comprises a plurality of guide nucleic acids, each guide nucleic acid of the plurality of guide nucleic acids is complementary, or partially complementary to a different segment of the target nucleic acid. In some embodiments, the method further comprises, prior to step (a): providing a collection tube comprising a sample solution comprising the target nucleic acid; and transferring the sample solution to the device via inserting the collection tube into a sample interface of the device, wherein the sample solution dissolves and mixes with a lyophilized reaction mix comprising amplification reagents. In some embodiments, the method may comprise all of the embodiments the various devices, as described herein.

Described herein are various devices for detecting a target nucleic acid, comprising: a sample interface configured to receive a sample comprising the target nucleic acid; a first reaction chamber in fluid communication with the sample interface, wherein the first reaction chamber comprises a plurality of reporters; and a second reaction chamber in fluid communication with the first reaction chamber, wherein the second reaction chamber comprises an enzyme substrate, wherein each reporter of the plurality of reporters (i) is immobilized to a surface of the reaction chamber via a tether, (ii) comprised of a programmable nuclease-enzyme fusion protein complexed with a guide nucleic acid, the guide nucleic acid being configured to bind to the target nucleic acid, and (iii) configured to release the programmable nuclease-enzyme fusion protein following cleavage of the tether by the programmable nuclease-enzyme fusion protein bound to the target nucleic acid, and wherein release of the programmable nuclease-enzyme fusion protein is indicative of a presence or absence of the target nucleic acid in the sample. In some embodiments, the programmable nuclease-enzyme fusion protein comprises Cas12-HRP. In some embodiments, the tether of each reporter of the plurality of reporters comprises ssDNA. In some embodiments, the tether of each reporter of the plurality of reporters comprises RNA. In some embodiments, the tether of each reporter of the plurality of reporters comprises both ssDNA and RNA. In some embodiments, the programmable nuclease-enzyme fusion protein comprises Cas12-HRP. In some embodiments, the tether of each reporter of the plurality of reporters comprises RNA. In some embodiments, the enzyme substrate is HRP substrate. In some embodiments, the surface is configured to immobilize a nucleic acid. In some embodiments, the surface comprises streptavidin, biotin, an amine group, a carboxyl group, an epoxy group, an NHS group, a malemide group, or a thiol group. In some embodiments, the surface is a hydrogel surface. In some embodiments, the surface comprises an acrydite modification.

Described herein are various devices for detecting a target nucleic acid, comprising: a sample interface configured to receive a sample comprising the target nucleic acid; a first reaction chamber in fluid communication with the sample interface, wherein the first reaction chamber comprises a plurality of reporters; a second reaction chamber in fluid communication with the first reaction chamber, wherein the second reaction chamber comprises an enzyme substrate, wherein each reporter of the plurality of reporters comprises a programmable nuclease-split enzyme fusion protein complexed with a guide nucleic acid, the guide nucleic acid being configured to bind to the target nucleic acid, and wherein release of the programmable nuclease-split enzyme fusion protein is indicative of a presence or absence of the target nucleic acid in the sample. In some embodiments, the programmable nuclease-split enzyme fusion protein comprises HRP-L. In some embodiments, the device further comprises a programmable nuclease-split enzyme fusion protein comprising HRP-S. In some embodiments, the device is configured to produce a signal upon binding of the programmable nuclease-split enzyme fusion protein comprising HRP-L and binding of the programmable nuclease-split enzyme fusion protein comprising HRP-S to the target nucleic acid.

Described herein are various devices for detecting a target nucleic acid, comprising: a sample interface configured to receive a sample comprising the target nucleic acid; a first reaction chamber in fluid communication with the sample interface, wherein the first reaction chamber comprises a plurality of reporters, each reporter of the plurality comprising two or more [enzymes]; and a surface of the first reaction chamber, wherein each reporter of the plurality of reporters is immobilized to the surface of the first reaction chamber via a tether, and a second reaction chamber in fluid communication with the first reaction chamber, wherein the second reaction chamber comprises an enzyme substrate, and wherein cleaving the tether by a programmable nuclease complexed with a guide nucleic acid, the guide nucleic acid being configured to bind to the target nucleic acid, may release the two or more enzymes into solution, and wherein each enzyme of the two or more enzymes may be contacted with the enzyme substrate in the second reaction chamber to create a detectable and amplified signal indicative of a presence or absence of the target nucleic acid in the sample. In some embodiments, each enzyme of the two or more enzymes comprises HRP. In some embodiments, the reporter comprises a streptavidin comprising the two or more enzymes. In some embodiments, the tether contacts the surface via a biotin, an acrydite, or an amine. In some embodiments, the surface of the first reaction chamber is functionalized with a hydrogel, carboxyl group, or N-Hydroxysuccinimide (NHS). In some embodiments, the [tether] comprises a segment of cleavable DNA, cleavable RNA or a combination thereof. In some embodiments, the enzyme substrate comprises 3,3′,5,5′-tetramethylbenzidine (TMB). In some embodiments, the surface of the first reaction chamber comprises an inner wall of the first reaction chamber. In some embodiments, the surface of the first reaction chamber comprises a surface of a bead, wherein the bead is contained within the first reaction chamber. In some embodiments, the bead is a magnetic bead.

In one aspect, the present disclosure provides a composition for detecting a target nucleic acid, the composition comprising a programmable nuclease, a guide nucleic acid, a first reporter, an enzyme, and a second reporter. In some embodiments, (a) the guide nucleic acid is configured to bind to the target nucleic acid; (b) the programmable nuclease is effective to cleave the first reporter in response to formation of a complex comprising the programmable nuclease, the guide nucleic acid, and the target nucleic acid; (c) the cleavage of the first reporter is effective to separate a first nucleic acid section from a second nucleic acid section thereof; (d) the first nucleic acid section is effective to activate the enzyme; and (e) the activated enzyme is effective to cleave the second reporter to produce a detectable product comprising a detection moiety. In some embodiments, the enzyme is an endonuclease (e.g., NucC) and the second reporter comprises a polynucleotide substrate of the enzyme (e.g., cyclic triadenylate (cA3)). In some embodiments, the second nucleic acid section comprises RNA residues, optionally wherein the RNA residues comprise a plurality of uracil residues. In some embodiments, the second nucleic acid section comprises DNA residues, optionally wherein the DNA residues comprise a plurality of thymine residues. In some embodiments, (a) the second reporter comprises a fluorescent label and a quencher, and (b) cleavage of the second reporter by the activated enzyme is effective to separate the fluorescent label from the quencher.

In one aspect, the present disclosure provides a method of detecting a target nucleic acid in a sample. In some embodiments, the method comprises (a) contacting the sample with a composition described herein (e.g., a composition comprising a programmable nuclease, a guide nucleic acid, a first reporter, an enzyme, and a second reporter); (b) cleaving the first reporter with the programmable nuclease in response to formation of the complex comprising the programmable nuclease, the guide nucleic acid, and the target nucleic acid, thereby releasing the first nucleic acid section; (c) activating the enzyme with the first nucleic acid section; (d) cleaving the second reporter with the activated enzyme, thereby producing the detectable product comprising the detection moiety; and (e) detecting the detection moiety, thereby detecting the presence of the target nucleic acid in the sample. In some embodiments, the second reporter comprises a polynucleotide substrate of the enzyme, and the enzyme is a NucC. In some embodiments, step (d) is performed at a temperature of at least 40° C. In some embodiments, all of steps (b)-(d) are performed at a temperature of at least 40° C. (e.g., 45° C., 50° C., 55° C., 60° C., 65° C., or higher).

In one aspect, the present disclosure provides a composition for detecting a target nucleic acid in a reaction chamber, the composition comprising a programmable nuclease, a guide nucleic acid, a forward primer, a reverse primer, a polymerase, a nicking endonuclease, and a reporter. In some embodiments, (a) the guide nucleic acid is configured to bind to the target nucleic acid; (b) the forward primer comprises (i) a 5′ portion comprising a first hairpin, and (ii) a 3′ portion that is configured to bind the target nucleic acid at a first overlapping region with respect to the guide nucleic acid; (c) the reverse primer comprises (i) a 5′ portion comprising a second hairpin, and (ii) a 3′ portion that is configured to bind a complement of the target nucleic acid at a second overlapping region with respect to the guide nucleic acid; (d) the first and second hairpins are cleavage substrates for the nicking endonuclease; (e) the programmable nuclease is effective to cleave the reporter in response to formation of a complex comprising the programmable nuclease, the guide nucleic acid, and (i) the target nucleic acid or (ii) an amplicon of the target nucleic acid; and (f) the cleavage of the reporter is effective to produce a detectable product comprising a detection moiety. In some embodiments, (a) the sequence of the target nucleic acid to which the 3′ portion of the first primer is configured to bind defines a first sequence of the target nucleic acid; (b) the sequence of the 3′ portion of the reverse primer defines a second sequence of the target nucleic acid; and (c) the first sequence and second sequence are separated by about 5 to about 10 nucleotides along the target nucleic acid. In some embodiments, the 3′ portions of the forward primer and reverse primer are about 16 to about 20 nucleotides in length. In some embodiments, overlap between the 3′ portion of the reverse primer and the sequence to which the guide nucleic acid is configured to bind overlap by 1 to 5 nucleotides, 2 to 5 nucleotides, or 3 nucleotides. In some embodiments, the first hairpin and/or the second hairpin are 10 to 20 nucleotides in length, 16 to 20 nucleotides in length, or 16 nucleotides in length. In some embodiments, the programmable nuclease is a Cas protein, optionally wherein the Cas protein is a Cas12 protein or a Cas14 protein.

In a related aspect, the present disclosure provides a method of detecting a target nucleic acid in a sample using a composition described herein (e.g., a composition comprising a programmable nuclease, a guide nucleic acid, a forward primer, a reverse primer, a polymerase, a nicking endonuclease, and a reporter). In some embodiments, the method comprises: (a) contacting the sample with the composition; (b) performing nicking enzyme amplification reaction (NEAR) reaction to amplify the target nucleic acid; (c) forming a complex comprising the programmable nuclease, the guide nucleic acid, and (i) the target nucleic acid, or (ii) an amplicon of the target nucleic acid; (d) cleaving the reporter with the programmable nuclease activated by formation of the complex, thereby producing the detectable cleavage product; and (e) detecting the detection moiety, thereby detecting the presence of the target nucleic acid in the sample. In some embodiments, steps (b) through (d) are performed at about the same temperature (e.g., as in an isothermal reaction).

In one aspect, the present disclosure provides systems comprising a reaction chamber comprising a composition described herein, such as with regard to any of the various aspects described herein. In some embodiments, the reaction chamber is a chamber within a device disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 shows a process flow chart for a programmable nuclease-based detection device, whereby a sample comprising one or more target sequences is collected and prepared before the one or more target sequences are detected. Sample preparation includes compartmentalized thermocycling.

FIGS. 2A-2B show top down and cross-section views of a programmable nuclease-based detection device, as described herein.

FIGS. 3A-3B illustrate a cross sectional view of a programmable nuclease-based detection device comprising a plurality of thermocycling compartments with movable mechanisms, as described herein.

FIGS. 4A-4B illustrate a programmable nuclease probe comprising a programmable nuclease and a guide nucleic acid complexed with the programmable nuclease before and after a complementary binding event, as described herein.

FIGS. 5A-5B show a programmable nuclease probe before and after a complementary binding event and the generation of a signal indicating a presence of a target sequence or target nucleic acid, as described herein.

FIG. 6 illustrates a large molecular weight reporter that can undergo a reaction to generate an amplified signal that is detectable by a sensor, as described herein.

FIGS. 7A-7C illustrate various multiplexing embodiments of the programmable nuclease-based detection device, as described herein.

FIG. 8 illustrates an assay design for a point-of-need (PON) 5-plex respiratory panel.

FIG. 9 illustrates a PON disposable device, according to an embodiment, as described herein.

FIGS. 10A-10C illustrate a consumable and embodiments of components, as described herein.

FIGS. 11A-11D further illustrate various embodiments of components of a consumable, as described herein.

FIG. 12 illustrates valve positioning of the consumable device, as described herein.

FIG. 13 presents an oxidation curve for HERC2 gene DETECTR reaction with electrochemical reporters, as described herein.

FIG. 14 presents a reduction curve for HERC2 DETECTR reaction with electrochemical reporters, as described herein.

FIG. 15 presents a cyclic voltammogram taken before and after HERC2 DETECTR reaction, as described herein.

FIG. 16 presents an oxidation curve for SARS-CoV-2 DETECTR reaction with electrochemical reporters, as described herein.

FIG. 17 presents full data set for square wave voltammetry measurements of SARS-CoV-2 DETECTR reaction with electrochemical reporters and controls, as described herein.

FIG. 18 presents complexing master mix with R1763 (N-gene), as described herein.

FIG. 19 presents experimental conditions for square wave voltammetry measurements, as described herein.

FIGS. 20A-20C illustrates immobilization strategies for CRISPR-Cas diagnostic assay components, as described herein

FIG. 21 illustrates an embodiment where immobilization strategies are combined to enable CRISPR diagnostic readouts, as described herein.

FIG. 22 presents results for the evaluation of the compatibility of various chemical modifications to gRNAs, as described herein.

FIG. 23 presents results for the immobilization of gRNAs to a streptavidin coated surface, as described herein.

FIG. 24 presents results for immobilization of Cas protein-RNA complexes, as described herein.

FIGS. 25A-25B present results for immobilization of reporters, as described herein.

FIG. 26 presents results for functional testing of combined immobilized ribonucleoprotein (RNP) and reporter system, as described herein.

FIGS. 27A-27E present results for evaluation of different reporters for immobilization in combination with Cas complex immobilization, as described herein.

FIGS. 28A-28C present results for Cy5 reporter is functional for DETECTR, as described herein.

FIGS. 29A-29F present results for immobilization optimization involving the complex formation step, as described herein.

FIGS. 30A-30B present results for immobilization optimization involving a gRNA/reporter binding time and reporter concentration.

FIGS. 31A-31C present results showing target discrimination of modified gRNAs.

FIGS. 32A-32E present results showing biotin-modified Cas13a gRNA is functional.

FIG. 33 presents results of a streptavidin coated microscope slide with biotinylated reporter.

FIGS. 34A-34B present results of DETECR reaction on glass slide.

FIG. 35 present experiment conditions, as described herein.

FIG. 36 present experiment conditions, as described herein. Figure discloses SEQ ID NOS 1455-1456, 1492 and 1518, respectively, in order of appearance.

FIGS. 37A-37B present experiment conditions, as described herein.

FIGS. 38A-38B present experiment conditions, as described herein.

FIGS. 39A-39B present experiment conditions, as described herein.

FIGS. 40A-40B present experiment conditions, as described herein.

FIGS. 41A-41B present experiment conditions, as described herein.

FIG. 42 presents experiment conditions, as described herein.

FIG. 43 shows a layout for a DETECTR assay device.

FIG. 44 shows a schematic of a sliding valve device.

FIG. 45 shows a diagram of sample movement through the sliding valve device as shown in FIG. 44.

FIG. 46 presents results for sample preparation optimization for an initial lysis and concentration buffer screen.

FIG. 47 presents results for sample preparation optimization involving Hotpot with Cas 14a.1

FIG. 48 presents results for sample preparation optimization involving a LANCR

(Cas12 variant DETECTR using SEQ ID NO: 17) control run.

FIGS. 49A-49B present lyophilization optimization results with Group 1: Trehalose using RT-LAMP on the left and DETECTR on the right.

FIGS. 50A-50B present lyophilization optimization results for Group 2: PVP 40, sorbitol, Mannitol, Mannosse, using RT-LAMP MM with 3-8% of the candidate excipient as shown in FIG. 50A and results for DETECTR as shown in FIG. 50B.

FIGS. 51A-51B presents lyophilization optimization results for Group 2: PVP 40, Sorbitol, Mannitol, Mannose, using DETECTR MM with 3-5% of candidate excipient.

FIG. 52 presents lyophilization optimization results for RT-LAMP Mastermix in Trehalose Functionality Screening.

FIGS. 53A-53B presents lyophilization optimization results for DETECTR Mastermix in Trehalose, as described herein.

FIGS. 54A-54B present results for HotPot involving LAMP amplification with Cas14a DETECTR in single reaction volume (one-pot).

FIG. 55 presents results for RT-LAMP amplification with Cas14a DETECTR in single reaction volume (one-pot).

FIGS. 56A-56B presents results for identifying buffers that are compatible with Cas14a and low temperature RT-LAMP (LowLAMP).

FIG. 57 presents results involving the impact of individual components on the performance of Cas 14 at low temperature RT-LAMP conditions.

FIG. 58 presents results for LAMP amplification with Cas14a DETECTR in single reaction volume (one-pot).

FIG. 59A-59B presents results for one-pot Cas14 with LowLAMP at 50 C.

FIG. 60 presents results for one-pot Cas14 with Bsm DNA polymerase at 55 C.

FIG. 61 presents results for a limit of detection study involving one-pot DETECTR (HotPot).

FIG. 62 presents results for a limit of detection study involving one-pot DETECTR (HotPot), where two different DNA polymerases at 55 C were tested.

FIG. 63 presents results for a study involving replacing Bst polymerase in the NEAR assay, showing enablement for SARS-CoV-2 detection at lower temperatures.

FIG. 64 presents results for NEAR assay amplification functions in Cas14a optimal buffers.

FIG. 65 presents results for Cas14a functions in a range of KOAc salt concentrations.

FIG. 66 presents results for a study involving increasing concentrations of KOAc to improve NEAR performance in Cas14a optimal buffers.

FIG. 67 presents results for a study involving increasing concentrations of KOAc to improve NEAR performance in Cas14a optimal buffers.

FIGS. 68A-68B present sequences and results for performance of Cas14a.1 crRNAs on SARS-CoV-2 E-gene amplicon, respectively. FIG. 68A discloses SEQ ID NOS 1519.

FIG. 69 presents results for the evaluation of the performance of Klenow (exo-) NEAR assay in IB13 buffer at decreasing salt concentrations.

FIG. 70 presents an overview of sRCA.

FIG. 71 presents results from screening dumbbell DNA templates for sRCA.

FIG. 72 presents results from a study involving the ability of Cas14a to detect product of RCA reaction across increasing temperatures.

FIG. 73 presents results from a study involving the effects of trigger oligos.

FIG. 74 presents results from a study involving a titration of trigger oligos for Cas14 one-pot sRCA.

FIG. 75 presents results from evaluating a Cas12 variant (SEQ ID NO: 17) in one-pot sRCA.

FIG. 76 presents an overview of RCA positive feedback for Cas13.

FIG. 77 presents results from evaluating Cas13-compatible DNA templates for RCA.

FIG. 78 presents results from a study evaluating whether a Cas13-compatible DNA template is functional in RCA.

FIG. 79 presents results from a study involving Cas13 functionality in a one-pot sRCA reaction across increasing temperatures.

FIG. 80 presents an overview of CasPin.

FIG. 81 presents potential hairpin structures for CasPin.

FIG. 82 presents results for an initial design using two hairpins. Figure discloses SEQ ID NO: 1520.

FIG. 83 presents a schematic of combined gRNA and reporter immobilization on the left and results for immobilization of DETECTR components using NHS-Amine chemistries on the right.

FIG. 84 presents results from optimizing the conjugation buffer to reduce non-specific binding.

FIG. 85 presents results from a study involving immobilizing different combinations of reporter+guide+Cas12 variant (SEQ ID NO: 17).

FIG. 86 presents results from a study optimizing gRNA and target concentrations to improve signal-to-noise ratio for immobilized DETECTR.

FIGS. 87A-87B present modifications and results from evaluating various amino modifications for DETECTR immobilization, respectively.

FIG. 88 presents results for the FASTR assay, involving detection of SARS-CoV-2 with rapid thermocycling+CRISPR Dx.

FIG. 89 presents results from a study to determine top performing polymerases and buffers for the FASTR assay.

FIG. 90 presents results for single copy detection of SARS-CoV-2 with FASTR.

FIG. 91 presents results for variations on rapid cycling times for denaturation and annealing/extension in FASTR.

FIG. 92 presents results for minimizing RT time for FASTR.

FIG. 93 presents results for higher pH buffers that improve FASTR performance.

FIG. 94 presents results for FASTR compatibility with crude lysis buffers.

FIG. 95 presents results for non-optimized multiplexing of FASTR.

FIG. 96 presents results for multiplex FASTR.

FIG. 97 presents results for the limit of detection of multiplex FASTR.

FIG. 98 presents key primers and gRNAs. Figure discloses SEQ ID NOS 1521-1529, respectively, in order of appearance.

FIGS. 99A-99B present results for a one-pot master mix of both RT-LAMP and DETECTR assay reagents pooled together. Aliquots of the same reaction mixture containing Cas12 variant (SEQ ID NO: 17) were run separately for each assay. FIG. 99A shows results for the RT-LAMP assay and FIG. 99B shows results for the DETECTR assay.

FIG. 100 presents results for the reconstituted Cas12-based DETECTR master mix after lyophilization (using a Cas12 variant (SEQ ID NO: 17)).

FIG. 101 presents results for a Cas14a1-based DETECTR assay.

FIGS. 102A-102B present results for RT-LAMP and Cas12-based DETECTR assays where one sample containing the reagent master mix was stored for two weeks prior to lyophilization (using a Cas12 variant (SEQ ID NO: 17)).

FIG. 103 presents results for small volume, lyophilized reaction master mixes.

FIG. 104 presents dynamic scanning calorimetry results for a pooled, lyophilized master mix of DETECTR and RT-LAMP reagents.

FIG. 105 presents a list of excipients.

FIG. 106 presents results for a one-pot DETECTR assay ran on a handheld microfluidic device.

FIGS. 107A-107B illustrate an embodiment of a multiplex lateral flow strip, as described herein.

FIG. 108 illustrates an embodiment of a workflow with multiplex “HotPot” as described herein.

FIG. 109 illustrates an embodiment for HRP paper-based detection, as described herein.

FIG. 110 illustrates an embodiment for an HRP-based multiplex lateral flow assay, as described herein.

FIG. 111 illustrates an embodiment for Multiplexed Cas13 immobilization approach to an HRP-based multiplex lateral flow assay, as described herein.

FIG. 112 shows results for both DNAse and DETECTR based assays for two replicate runs a week apart.

FIGS. 113A-113B illustrate the use of multiple Cas-complex probes guide pooling enhanced signal detection to a lateral flow assay, as described herein.

FIG. 114 depicts results of a DETECTR assay showing enhanced Cas12a-based detection of the GF184 target using a pooled-guide (pooled-gRNA) format compared to DETECTR Cas12a-based assay using an individual gRNA format.

FIG. 115 depicts results of a DETECTR assay showing enhanced sensitivity of the Cas13a-based detection of the SC2 target using a pooled-guide format compared to the Cas13a-based assays using an individual guide format.

FIG. 116 shows images corresponding to each chamber, used to count the number of positive droplets, showing that the Cas13a-DETECTR assay samples containing the pooled guide RNAs generated more crystals containing the amplified products per starting copy of the target RNA than the Cas13a-DETECTR assay samples containing the guide RNAs in individual format.

FIG. 117 shows that measurement of signal intensity following amplification showed that the Cas13a-DETECTR assay samples containing the pooled guide RNAs generated more signal intensity per starting copy of the target template RNA than the Cas13a-DETECTR assay samples containing the guide RNAs in individual format.

FIG. 118 shows that measurement of signal intensity following amplification showed that the Cas13a-DETECTR assay samples containing the pooled guide RNAs generated more signal intensity per starting copy of the target template RNA than the Cas13a-DETECTR assay samples containing the guide RNAs in individual format. FIG. 118 also shows that relative quantification performed by counting the number of positive droplets showed that the Cas13a-DETECTR assay samples containing the pooled guide RNAs generated more crystals containing the amplified products per starting copy of the target template RNA than the Cas13a-DETECTR assay samples containing the guide RNAs in individual format.

FIG. 119 shows that Cas13a DETECTR assay samples containing the pooled guides (R4637, R4638, R4667, R4676, R4684, R4689, R4691) did not exhibit higher target detection sensitivity per starting copy of the target than the Cas13a DETECTR samples containing the single guides R4684, R4667, or R4785 (RNAseP guide) in individual format.

FIG. 120 illustrates an embodiment of a handheld device comprising a lateral flow assay, as described herein.

FIGS. 121A-121B illustrate an embodiment of a point of care device comprising a lateral flow assay, as described herein.

FIG. 122 illustrates an embodiment of a point of care device comprising a lateral flow assay, as described herein.

FIG. 123 illustrates an embodiment of a point of care device comprising a lateral flow assay and a spiral reaction chamber as described herein.

FIG. 124 illustrates an embodiment of a point of care device comprising switch triggers for triggering device actuators as described herein.

FIG. 125 illustrates an embodiment of a reaction chamber that is coupled to an input port and is substantially spiral in shape as described herein.

FIG. 126 illustrates an embodiment of a housing structure for lateral flow assays comprising channel structures of substantially the same contour length for each assay as described herein.

FIGS. 127A-127B illustrates an embodiment of a point of care device before actuation and after partial actuation of device actuators as described herein.

FIGS. 128A-128B illustrates an embodiment of a chemical heating element and its time-dependent temperature profile as described herein.

FIGS. 129A-129B illustrates embodiments of lateral flow strips configurations as described herein.

FIG. 130 illustrates an embodiment of a point of care device comprising a chemical heating element and an electrical heating element.

FIGS. 131A-131B illustrates results of DETECTR assays showing successful nucleic acids amplification utilizing one of the point of care device embodiments described herein.

FIG. 132 illustrates results of DETECTR assays showing successful detection of nucleic acids utilizing one of the flow strip assay embodiments described herein.

FIG. 133 illustrates a flow diagram of a process used to evaluate, characterize, and optimize proteins for diagnostic applications.

FIG. 134 illustrates a schematic showing a workflow for the process using Labcyte Echo.

FIG. 135 shows experimental results for fluorescence of three candidate Cas enzymes.

FIG. 136 shows the performance of three candidate Cas enzymes at different temperatures and buffers.

FIG. 137 shows the results of testing conducted with CasM. 1740 with three additional buffers at 35° C.

FIG. 138 shows the results of experiments investigating the limits of detection on single-strand oligo or synthetic dsDNA target at 35° C.

FIG. 139 shows the results of experiments evaluating the limit of detection at both 35° C. and the highest temperature that the protein was demonstrated to function at for CasM.1740.

FIG. 140 shows the results of experiments investigating the effects of additives and assay formulations on the performance of the proteins.

FIG. 141 shows the results for single nucleotide mutation sensitivity experiments with CasM.124070.

FIG. 142 shows the results for single nucleotide mutation sensitivity experiments with CasM.08.

FIG. 143 shows the results for single nucleotide mutation sensitivity experiments with CasM.124070 and CasM.08 with the same target site.

FIGS. 144A-144B show the results of experiments investigating the kinetics of trans-cleavage for proteins that have the best performance in terms of sensitivity, specificity, or thermostability.

FIG. 145 summarizes the performance results for various Cas enzymes.

FIG. 146 shows a schematic of an exemplary workflow for a multiplexed programmable nuclease assay.

FIGS. 147 and 148 show an exemplary, a non-limiting handheld device for performing a multiplexed programmable nuclease (e.g., DETECTR) assay.

FIG. 149 shows an exemplary hydrogel comprising immobilized reporters co-polymerized therein.

FIGS. 150A and 150B show exemplary multiplexing strategies for hydrogel immobilized DETECTR systems.

FIGS. 151A-151B show an exemplary positive feedback system for signal amplification.

FIG. 152 shows an exemplary workflow for DETECTR-based HotPot reactions.

FIG. 153 shows fluorescence results of HotPot reactions with reporters immobilized on glass beads.

FIG. 154 shows lateral flow strip results using samples from the same experiments conducted to yield results illustrated in FIG. 153.

FIG. 155 shows fluorescence results of HotPot reactions with reporters immobilized on magnetic beads.

FIG. 156 shows fluorescence results of DETECTR-based HotPot assays for a variety of respiratory disease nucleic acid sequence targets.

FIGS. 157A-157C shows results of limit of detection experiments for initial HotPot assay testing.

FIG. 158 shows the fluorescence detected from HotPot assays in the presence of various additives.

FIGS. 159A-159B shows the influence of select additives that increase the speed and/or the signal strength of some HotPot assays.

FIG. 160 shows HotPot results from experiments conducted with various amounts of a few additives.

FIG. 161 shows HotPot results from experiments conducted with various amounts of a few additives with BSM DNA Polymerases.

FIGS. 162A-162B show lateral flow assay results of DETECTR-based OnePot and HotPot assays conducted with hydrogels comprising immobilized reporters.

FIGS. 163A-163D illustrate a non-limiting example of a handheld device for performing a multiplexed programmable nuclease (e.g., DETECTR) assay.

FIG. 164 presents results depicting the enhancement of the signal generated in a Hotpot reaction with the addition of Thermostable Inorganic Pyrophosphatase (TIPP) in comparison to control conditions lacking TIPP, target RNA or both.

FIGS. 165A-165B present results from DETECTR lateral flow Hotpot reaction assay strips, depicting the enhancement of the signal generated in a Hotpot reaction with the addition of Thermostable Inorganic Pyrophosphatase (TIPP) in comparison to control conditions lacking TIPP, target RNA or both.

FIG. 166 shows a schematic of the NEAR reaction. A forward and reverse primer consisting of a nicking enzyme stabilization site and recognition region able amplify a target region of interest into a single-stranded DNA molecule. A guide RNA will bind a region complementary to the amplified ssDNA, allowing further detection by a DETECTR system.

FIGS. 167A-167B show an exemplary NEAR-DETECTR reaction. FIG. 167A show a forward and reverse primer flanking the target region. In addition, a panel of 19 guide RNAs are shown in comparison to the amplicon. FIG. 167A discloses SEQ ID NOS 53, 1530 and 54, respectively, in order of appearance. FIG. 167B shows the detection of the amplicon using NEAR-DETECTR using the above mentioned guide RNAs.

FIGS. 168A-168B shows an example NEAR reaction with an exemplary guide RNA showing detection of the E-gene of SARS-CoV2. FIG. 168A shows an example NEAR reaction with forward and reverse NEAR primers with a guide RNA. FIG. 168A discloses SEQ ID NO: 1531. FIG. 168B shows the NEAR-DETECTR reaction following amplification, showing the resulting signal of 20,000 copies or 0 copies of the amplicon in solution.

FIG. 169 shows the resulting signal of a NEAR-DETECTR reaction in which the pre-amplification time is varied prior to DETECTR.

FIG. 170 shows the comparison of the NEAR-DETECTR reaction using orthogonal Cas systems (Cas12 Variant (SEQ ID NO: 17), Cas13 Variant (SEQ ID NO: 21), and Cas14 Variant (SEQ ID NO. 3)). The experiments were performed in the presence or absence of the target NEAR amplicon, showing different cleavage preferences for the reporter molecule.

FIG. 171 shows the optimization of the NEAR reaction using different magnesium (Mg2+) concentrations. This shows the time to result (in minutes) comparing different buffer compositions and different concentrations of added magnesium. The top panel shows the following conditions using Bst2.0, and the bottom panel shows the following experimental conditions using Bst.3.0. These results informed the following experimental conditions for the NEAR reaction: Bst2.0, 12 mM Mg2+, at 60 C, resulting in a less than 5 minute amplification time and approximately 20,000 copies.

FIGS. 172A-172B shows the experimental design of the primer and guide RNA design and the results of these designs. In FIG. 172A, eight primer pairs (R1763 F (1-8)/R (1-8)) and the guide RNA R1763. FIG. 172A discloses SEQ ID NO: 1532. In FIG. 172B, the aforementioned primers pairs and guides R1763 and R1765 are used in a NEAR-DETECTR reaction to determine the efficacy of the reaction using different primer pairs. The raw fluorescence (AU) of the NEAR-DETECTR reaction is reported.

FIGS. 173A-173B describes the optimization of the hinge stabilization region in the NEAR primers to determine if the nicking enzyme activity can be modulated. In FIG. 173A, the hinge stabilization loop region is modified to alter the melting temperature of the stem loop. In FIG. 173B, the modified stem loops as shown in FIG. 173A were used to for detection of the SARS-CoV-2 E-gene using different inputs of amplicons resulting from a NEAR amplification (1,000, 500, and 200 input amplicon copies).

FIG. 174 shows a comparison of a reverse transcription-NEAR-DETECTR (RT-NECTR) reaction using different reverse transcriptases. Wartmstart RTx (NEB), Bst 3.0, and Omniscript RT (Qiagen) were used on different amounts of input RNA in order to compare the limit of detection (LOD) of these reaction conditions.

FIG. 175 shows the experimental results of an RT-NECTR reaction using differing concentrations of NEAR primers.

FIG. 176 shows the LOD of the E-gene of SARS-CoV-2 using RT-NECTR using different amplification times and input copies for the RT-NECTR reaction.

FIG. 177 shows detection of the SARS-CoV-2 E gene using different Cas systems (Cas12 Variant (SEQ ID NO: 17), Cas13 Variant (SEQ ID NO: 21), and Cas14 Variant (SEQ ID NO. 3)) and a panel of different guides (SEQ ID NO: 76-SEQ ID NO: 82).

FIG. 178 shows an exemplary, a non-limiting handheld device for performing a programmable nuclease (e.g., DETECTR) assay.

FIG. 179 shows a cross-sectional view of the device of FIG. 178.

FIG. 180 shows a reaction channel component of the device of FIG. 178.

FIG. 181 shows an isometric view of a detection pen of the device of FIG. 178.

FIG. 182 shows a cross-sectional view of the detection pen of FIG. 181.

FIG. 183 shows an exploded view of the detection pen of FIG. 181.

FIG. 184 shows a cross-sectional view of a sample preparation reservoir of the device of FIG. 178.

FIG. 185 shows a point of care device for performing a multiplexed programmable nuclease (e.g., DETECTR) assay.

FIG. 186 shows an exploded view of device of FIG. 185 with fluidic chip assembly disposed between chemical heating packs.

FIGS. 187A-187C show various views of the chip assembly of the device of FIG. 185. FIG. 187A shows a perspective view of a chip assembly comprising a fluidic chip, a plunger assembly, and a detection clip. FIG. 187B shows a top view of the chip assembly of FIG. 187A. FIG. 187C shows a front view of a detection card configured to be inserted into a detection interface of the detection clip of FIG. 187A.

FIG. 188 shows side and top views of the fluidic chip of FIG. 187A.

FIGS. 189A-189C show front (FIG. 189A), side (FIG. 189B), and cross-sectional (FIG. 189C) vies of the fully assembled chip assembly of FIG. 185 including a fluidic chip, a plunger assembly, a detection clip, and a detection card.

FIGS. 190A-190D show perspective (FIG. 190A), top (FIG. 190B), side (FIG. 190C), and bottom (FIG. 190D) views showing various configurations of the fluidic chip and plunger assembly in use. Prior to use, the plunger assembly may be disposed within the fluidic chip in an initial configuration as shown in the middle row. After the sample is added to the sample interface, the plunger assembly may be pulled back into a loaded configuration to draw the sample into the reaction chamber/channel as shown in the top row. After the amplification and/or DETECTR reactions are run, the plunger assembly may be pushed into a dispensing configuration to push the sample out of the reaction chamber/channel and into the sample interface/detection interface as shown in the bottom row. The detection card comprising lateral flow assay strips may then be inserted into the detection interface and the detection region may be visualized to determine if the target nucleic acid was present in the sample as described herein.

FIGS. 191A-191C show cross-sectional side (left) and top (right) views highlighting various configurations of the fluidic chip and plunger assembly in use. Prior to use, the plunger assembly may be disposed within the reaction chamber/channel of the fluidic chip in an initial configuration as shown in FIG. 191B. After the sample is added to the sample interface, the plunger assembly may be pulled back into a loaded configuration to draw the sample into the reaction chamber/channel as shown in FIG. 191A. A locking mechanism may prevent the plunger assembly from moving during the reaction. After the amplification and/or DETECTR reactions are run, the plunger assembly may be pushed into a dispensing configuration to push the sample out of the reaction chamber/channel and into the sample interface/detection interface as shown in FIG. 191C. A locking release mechanism may be actuated (e.g., by squeezing) to release the locking mechanism and allow the plunger assembly to move between configurations. The detection card comprising lateral flow assay strips may then be inserted into the detection interface and the detection region may be visualized to determine if the target nucleic acid was present in the sample as described herein.

FIGS. 192A-192B show a signal amplification strategy involving the release of a tethered enzyme by a programmable nuclease. Presence of a target nucleic acid may activate the programmable nuclease, which may cleave a nucleic acid tether that immobilizes the enzyme on a surface. Cleaving the nucleic acid tether may release the enzyme into solution, which may be contacted with an enzyme substrate to create a detectable and amplified signal.

FIGS. 193A-193C show a signal amplification strategy involving the release of a programmable nuclease-enzyme fusion protein. Presence of a target nucleic acid may activate a programmable nuclease portion of the programmable nuclease-enzyme fusion protein, wherein the activated programmable nuclease may cleave a nucleic acid tether that immobilizes the programmable nuclease-enzyme fusion protein on a surface. Cleaving the nucleic acid tether may release the programmable nuclease-enzyme fusion protein into solution, wherein an enzyme portion of the programmable nuclease-enzyme fusion protein may be contacted with an enzyme substrate to create a detectable and amplified signal. In some cases, contacting may comprise contacting the solution comprising the released programmable nuclease-enzyme fusion protein with another solution comprising the enzyme substrate.

FIG. 194 shows a signal generation and amplification strategy involving the binding of two programmable nuclease-split enzyme fusion proteins with a target nucleic acid, wherein the two programmable nuclease-split enzyme fusion proteins each comprise an inactivated programmable nuclease portion and a split enzyme subunit portion. The programmable nuclease portion of each programmable nuclease-split enzyme fusion protein may be configured to bind to a different portion of a target nucleic acid. When the inactivated programmable nuclease portion of each programmable nuclease-split enzyme fusion protein binds to the target nucleic acid, the split enzyme subunit portion of each programmable nuclease-split enzyme fusion protein may dock to form an active enzyme. The active enzyme may be contacted with an enzyme substrate to create a detectable and amplified signal.

FIGS. 195A-195E show various exemplary reporters for use in the signal amplification strategy shown in FIGS. 192A-192B. Each reporter comprises at least one of the following: a detection moiety comprising a horseradish peroxidase (HRP) enzyme for signal enhancement, a nucleic acid-based linker-section, and a functionality for immobilization to a surface. In some embodiments, a carbon spacer may be positioned between the functionality and the nucleic acid and/or detection moiety. In FIG. 195A (e.g., rep161), the reporter comprises an enzyme (e.g., an HRP enzyme), a nucleic acid-based linker (e.g., a single-stranded nucleic acid comprising 20 thymines (T20) (SEQ ID NO: 126)), and a biotin functionality. In FIG. 195B (e.g., rep194), the reporter comprises an enzyme (e.g., an HRP enzyme), a nucleic acid-based linker (e.g., a single-stranded nucleic acid comprising 12 thymines (T12 (SEQ ID NO: 127)) each on either side of a cleavable section comprising 5 uracils), a carbon spacer, and a biotin functionality. In FIG. 195C (e.g., rep188), the reporter comprises an enzyme (e.g., an HRP enzyme), a nucleic acid-based linker (e.g., a single-stranded nucleic acid comprising 20 thymines (T20) (SEQ ID NO: 126)), and an acrydite functionality. In FIG. 195D (e.g., rep190), the reporter comprises an enzyme (e.g., an HRP enzyme), a nucleic acid-based linker (e.g., a single-stranded nucleic acid comprising 20 thymines (SEQ ID NO: 126)), a carbon spacer, and an amine functionality. In FIG. 195E (e.g., rep197), the reporter comprises an enzyme (e.g., a poly-HRP enzyme), a first carbon spacer, a nucleic acid-based linker (e.g., a single-stranded nucleic acid comprising 20 thymines (T20) (SEQ ID NO: 126)), a second carbon spacer, and an amine functionality. In FIG. 195E (e.g., rep197), a poly-HRP detection moiety (e.g., streptavidin (SA) molecule functionalized with multiple HRP groups) is conjugated to the nucleic acid linker via a biotin and the first carbon spacer. In at least some instances, the incorporation of multiple enzymes on one reporter may provide an assay signal boost upon a single cleavage event of the reporter induced by the activated programmable nuclease. In some embodiments, a linker comprises at least one linker segment, the functionality for immobilization to a surface, or a combination thereof. In some embodiments, the linker is referred to as a tether.

FIG. 196 shows a schematic of a reporter immobilized to a surface of a carboxylic acid magnetic bead (MB) via an amide bond. The depicted reporter comprises a poly-HRP reporter and may be substantially similar to the reporter shown in FIG. 195E.

FIGS. 197A-197E show a schematic (FIG. 197A) and results for experiments designed to study effects of reporter concentration (FIGS. 197B-197D) and to determine the limit of detection (FIG. 197E) of a programmable nuclease-based detection assay using reporters comprising HRP-based detection moieties (rep194) which were immobilized on streptavidin beads.

FIGS. 198A-198E show results for an experiment designed to study the effect of different concentrations of reporter functionalized to a plate (FIGS. 198A-198C and 198E) and to determine the limit of detection (FIG. 198D) of a programmable nuclease-based detection assay using reporters comprising HRP-based detection moieties (rep194) immobilized on a plate.

FIGS. 199A-199F show the results of a programmable nuclease-based detection assay utilizing hydrogels of different PEG-DA concentrations (20% or 35%) co-polymerized with different concentrations (10 uM or 1 uM) of reporters comprising HRP-based detection moieties and acrydite functionalities (rep188), where the hydrogels act as a solid support for the reporter comprising HRP signal enhancement enzymes.

FIGS. 200A-200F show a schematic (FIG. 200A) and results (FIGS. 200B-200F) of a programmable nuclease-based detection assay utilizing NHS-activated resin as the solid support for a reporter comprising an HRP signal enhancement enzyme (rep190).

FIGS. 201A-201D show results of a programmable nuclease-based detection assay using a poly-HRP reporter immobilized carboxylic beads as shown in FIG. 196. The bead volume and target concentration were varied to determine a limit of detection (LOD).

FIG. 202 shows a schematic of a signal amplification strategy involving downstream activation of an endonuclease following activation of a programmable nuclease in response to a target nucleic acid. Presence of the target nucleic acid may activate the programmable nuclease, which may cleave the first nucleic acid section of the first reporter from the second nucleic acid section of the first reporter, thereby freeing the first nucleic acid section to bind to and activate the endonuclease. The activated endonuclease may then cleave the second reporter and release a detection moiety that may be detected as described herein.

FIG. 203 shows illustrative structures for first reporters comprising first and second nucleic acid sections in accordance with some embodiments, such as the strategy illustrated in FIG. 202.

FIG. 204 shows a schematic of a signal amplification strategy involving reporters comprising an enzyme conjugated to a linker, in which the linker comprises a functionality for immobilization to a support. Presence of a target nucleic acid may activate the programmable nuclease, which may cleave a nucleic acid tether to separate the enzyme from the functionality for immobilization. The solution is then contacted with beads that bind the functionality for immobilization. Enzymes cleaved from the nucleic acid tether remain free, whereas in the absence of cleavage, enzymes become complexed with the beads. The solution is separated from the beads and tested for enzyme activity in the presence of enzyme substrate to create a detectable and amplified signal indicative of the presence of the target nucleic acid.

FIG. 205 shows illustrative results for assays with different reporter concentrations. Error bands indicate 1 standard deviation (n=3).

FIG. 206 shows illustrative results for assays with different beads and binding buffers. Error bands indicate 1 standard deviation (n=3).

FIG. 207 shows illustrative results for assays with reporters having tethers of different lengths. DNase is shown as a control for maximal activity of each reporter. Error bands indicate 1 standard deviation (n=3).

FIG. 208 shows illustrative results for assays with different reporter concentrations and bead volumes. Error bands indicate 1 standard deviation (n=3).

FIG. 209 shows illustrative results for a study of assay sensitivity for various concentrations of target. Error bands indicate 1 standard deviation (n=22). OTC indicates off-target control.

FIG. 210 shows a schematic representation of a process for concentrating enzymes released from a reporter (e.g., HRP) in an assay.

FIG. 211 shows illustrative results for the effects of concentrating enzymes released from a reporter in an assay. Error bands indicate 1 standard deviation (n=3).

FIG. 212 shows illustrative results for the effects of adding enzyme substrate serially following concentration of the enzyme on a substrate resin.

FIG. 213 shows illustrative results measured by raw fluorescence signal over reaction time for the effects of running a NucC enzyme activity assay at elevated temperatures with and without activator present during the reaction.

FIG. 214 shows a schematic representation of a surface-immobilized positive-feedback system.

FIG. 215 shows a schematic representation of a surface-immobilized positive-feedback system wherein the Cas #1 is initially in the solution and Cas #2 is initially immobilized. In a subsequent step, Cas #1 forms an activated complex upon binding to a first target nucleic acid and cleaves the ssDNA tether linking Cas #2 to the surface. The released Cas #2 is activated upon complexing with a second target nucleic acid (immobilized target #2), and the activated Cas #2 is effective to release additional immobilized Cas #2 and to cleave the reporter to produce a detectable product.

FIG. 216 shows graphical representations of the data output of a surface-immobilized positive feedback system assay using two programmable nucleases with one initially immobilized on a surface.

FIG. 217 shows a schematic representation of tethered guide-reporter chimera hybrid molecules with HRP wherein the tethered guide-reporter hybrid molecule is anchored on a surface with a carbon spacer connecting the programmable nuclease to the surface, and then a subsequent carbon spacer connecting the programmable nuclease to the reporter DNA, followed by a final carbon spacer linking the target DNA to HRP.

FIG. 218 shows illustrative results measured by raw fluorescence signal over reaction time for the effects of running Cas enzyme variants (Cas12 variants of SEQ ID NO: 17 or 34, or variants designated as a Cas12 variant having SEQ ID NO: 120, and a Cas12 variant having SEQ ID NO: 116) in a NEAR buffer conditions using Cas complexing and DETECTR.

FIG. 219 shows illustrative results measured by raw fluorescence signal over reaction time for the effects of running Cas variants (Cas of SEQ ID NO: 34, and a Cas12 variant having SEQ ID NO: 116) when added to a NEAR reaction mix.

FIG. 220 shows illustrative results measured by raw fluorescence signal over reaction time comparing a two-step NEAR reaction in the presence of guide alone (no template control, NTC) and presence of a template.

FIG. 221A-221B shows illustrative results measured by raw fluorescence signal over reaction time for primer screens of illustrative target sites R8895 (in influenza B virus (IBV)) (FIG. 221A) and R288 (in RSV-A) (FIG. 221B). Two-digit numbers along the top and right of the collections of graphs identify primers in the respective reactions. The sequences and locations of these primers are illustrated in FIGS. 222A-222B.

FIGS. 222A-222B shows a diagram depicting single base pair shifts of primers in an illustrative optimization of guide overlap for R8895 (FIG. 222A) and R288 (FIG. 222B). FIG. 222A discloses SEQ ID NOS 1533-1541, respectively, in order of appearance. FIG. 222B discloses SEQ ID NOS 1542-1545, 1487-1489 and 1450-1451, respectively, in order of appearance.

FIG. 223 shows illustrative results measured by raw fluorescence signal over reaction time for a NEAR reaction with three selected primer pairs of 25 with 24, 25 with 26, and 31 with 36 (referring to primers as numbered in FIG. 222A-222B).

FIG. 224 shows illustrative results measured by raw fluorescence signal over reaction time for a NEAR reaction with primer set 25-26 for the effect of TIPP on the reaction.

FIG. 225 shows illustrative results measured by raw fluorescence signal over reaction time for testing Savinase for RNAse activity.

FIG. 226 shows illustrative results measured by raw fluorescence signal over reaction time for testing Savinase for RNAse activity in combination with Savinase inhibitor phenylmethylsulfonyl fluoride (PMSF).

FIG. 227 shows illustrative results measured by raw fluorescence signal over reaction time for testing protease inhibitors for activity against CasM.26.

FIGS. 228A-228D show illustrative results measured by raw fluorescence signal over reaction time for testing Savinase under different conditions for protease activity.

FIGS. 229A-229D show illustrative results measured by raw fluorescence signal over reaction time for testing Savinase under different conditions for testing remaining RNAse activity.

FIGS. 230A-230B show illustrative results measured by raw fluorescence signal over time for the effects of running a NucC enzyme activity assay with and without various concentrations of linear activator present during the reaction.

### DETAILED DESCRIPTION

The present disclosure provides systems and methods for nucleic acid target detection. The systems and methods of the present disclosure can be implemented using devices that are configured for programmable nuclease-based detection. In some embodiments, the devices can be configured for single reaction detection. In some embodiments, the devices can be disposable devices. The devices disclosed herein can be particularly well suited for carrying out highly efficient, rapid, and accurate reactions for detecting whether a target is present in a sample. The target can comprise a target sequence or target nucleic acid. As used herein, a target can be referred to interchangeably as a target nucleic acid. Further, a target can be referred to as a target amplicon or a target nucleic acid amplicon if such target undergoes amplification (e.g., through a thermocycling process as described elsewhere herein). The target nucleic acid can be a portion of a nucleic acid of interest, e.g., a target nucleic acid from any plant, animal, virus, or microbe of interest. The devices provided herein can be used to perform rapid tests in a single integrated system.

The target nucleic acid can be a nucleic acid or a portion of a nucleic acid from a pathogen, virus, bacterium, fungi, protozoa, worm, or other agent(s) or organism(s) responsible for and/or related to a disease or condition in living organisms (e.g., humans, animals, plants, crops, and the like). The target nucleic acid can be a nucleic acid, or a portion thereof. The target nucleic acid can be a portion of a nucleic acid from a gene expressed in a cancer or genetic disorder in the sample. The target nucleic acid can be a portion of an RNA or DNA from any organism in the sample. In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter. The programmable nuclease can be referred to as an RNA-activated programmable RNA nuclease. In some instances, the programmable nuclease as disclosed herein can bind to a target DNA to initiate trans cleavage of an RNA reporter. Such a programmable nuclease can be referred to herein as a DNA-activated programmable RNA nuclease. In some cases, a programmable nuclease as described herein can be activated by a target RNA or a target DNA. For example, a programmable nuclease, e.g., a Cas enzyme, can be activated by a target RNA nucleic acid or a target DNA nucleic acid to cleave RNA reporters. In some embodiments, the Cas enzyme can bind to a target ssDNA which initiates trans cleavage of RNA reporters. In some instances, a programmable nuclease as disclosed herein can bind to a target DNA to initiate trans cleavage of a DNA reporter, and this programmable nuclease can be referred to as a DNA-activated programmable DNA nuclease.

The nucleic acids described and referred to herein can comprise a plurality of base pairs. A base pair can be a biological unit comprising two nucleobases bound to each other by hydrogen bonds. Nucleobases can comprise adenine, guanine, cytosine, thymine, and/or uracil. In some cases, the nucleic acids described and referred to herein can comprise different base pairs. In some cases, the nucleic acids described and referred to herein can comprise one or more modified base pairs. The one or more modified base pairs can be produced when one or more base pairs undergo a chemical modification leading to new bases. The one or more modified base pairs can be, for example, Hypoxanthine, Inosine, Xanthine, Xanthosine, 7-Methylguanine, 7-Methylguanosine, 5,6-Dihydrouracil, Dihydrouridine, 5-Methylcytosine, 5-Methylcytidine, 5-hydroxymethylcytosine (5 hmC), 5-formylcytosine (5fC), or 5-carboxylcytosine (5caC).

The programmable nuclease can become activated after binding of a guide nucleic acid that is complexed with the programmable nuclease with a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of detector nucleic acids with a detection moiety. Once the target nucleic acid is cleaved by the activated programmable nuclease, the detection moiety can be released or separated from the reporter and can directly or indirectly generate a detectable signal. The reporter and/or the detection moiety can be immobilized on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

The systems and methods of the present disclosure can be implemented using a device that is compatible with any type of programmable nuclease that is human-engineered or naturally occurring. The programmable nuclease can comprise a nuclease that is capable of being activated when complexed with a guide nucleic acid and a target nucleic acid segment or a portion thereof. A programmable nuclease can become activated when complexed with a guide nucleic acid and a target sequence of a target gene of interest. The programmable nuclease can be activated upon binding of a guide nucleic acid to a target nucleic acid and can exhibit or enable trans cleavage activity once activated. In any instances or embodiments where a CRISPR-based programmable nuclease is described or used, it is recognized herein that any other type of programmable nuclease can be used in addition to or in substitution of such CRISPR-based programmable nuclease.

The systems and methods of the present disclosure can be implemented using a device that is compatible with a plurality of programmable nucleases. The device can comprise a plurality of programmable nuclease probes comprising the plurality of programmable nucleases and one or more corresponding guide nucleic acids. The plurality of programmable nuclease probes can be the same. Alternatively, the plurality of programmable nuclease probes can be different. For example, the plurality of programmable nuclease probes can comprise different programmable nucleases and/or different guide nucleic acids associated with the programmable nucleases.

As used herein, a programmable nuclease generally refers to any enzyme that can cleave nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpf1. Programmable nucleases can also include, for example, PfAgo and/or NgAgo.

ZFNs can cut genetic material in a sequence-specific matter and can be designed, or programmed, to target specific viral targets. A ZFN is composed of two domains: a DNA-binding zinc-finger protein linked to the Fokl nuclease domain. The DNA-binding zinc-finger protein is fused with the non-specific Fokl cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) can cut genetic material in a sequence-specific matter and can be designed, or programmed, to target specific viral targets. TALENs contain the Fokl nuclease domain at their carboxyl termini and a class of DNA binding domains known as transcription activator-like effectors (TALEs). TALENs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target various nucleic acids.

The programmable nuclease can comprise any type of human engineered enzymes. Alternatively, the programmable nuclease can comprise CRISPR enzymes derived from naturally occurring bacteria or phage. A programmable nuclease can be a Cas protein (also referred to, interchangeably, as a Cas nuclease). A crRNA and Cas protein can form a CRISPR enzyme. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The programmable nuclease can comprise one or more amino acid modifications. The programmable nuclease be a nuclease derived from a CRISPR-Cas system. The programmable nuclease can be a nuclease derived from recombineering.

### Programmable Nucleases

Disclosed herein are programmable nucleases and uses thereof, e.g., detection and editing of target nucleic acids. In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide RNAs of Type V CRISPR/Cas proteins hybridize to ssDNA or dsDNA. However, the trans cleavage activity of Type V CRISPR/Cas protein is typically directed towards ssDNA. In some cases, the Type V CRISPR/Cas protein comprises a catalytically inactive nuclease domain. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease. The Type V CRISPR/Cas protein may be a Cas14 protein. The Cas 14 protein may be a Cas14a. 1 protein. The Cas14a. 1 protein may be represented by SEQ ID NO: 3, presented in Table 1. The Cas14 protein may comprise an amino acid sequence that is at least 70%, at least 75%, at least 80%, at least 85%, at least 90%, at least 95%, at least 98%, at least 99%, or 100% identical to SEQ ID NO: 3. The Cas14 protein may consist of an amino acid sequence that is at least 70%, at least 75%, at least 80%, at least 85%, at least 90%, at least 95%, at least 98%, at least 99%, or 100% identical to SEQ ID NO: 3. The Cas14 protein may comprise at least about 50, at least about 100, at least about 150, at least about 200, at least about 250, at least about 300, at least about 350, at least about 400, at least about 450, at least about 500 consecutive amino acids of SEQ ID NO: 3.

Table 1 provides illustrative amino acid sequences of programmable nucleases having trans-cleavage activity. In some instances, programmable nucleases described herein comprise an amino acid sequence that is at least 65%, at least 70%, at least 75%, at least 80%, at least 85%, at least 90%, at least 95%, at least 97%, or at least 98%, at least 99%, or 100% identical to any one of SEQ ID Nos: 1-34 or 87-125. The programmable nuclease may consist of an amino acid sequence that is at least 65%, at least 70%, at least 75%, at least 80%, at least 85%, at least 90%, at least 95%, at least 98%, at least 99%, or 100% identical to any one or SEQ ID Nos: 1-34 or 87-125. The programmable nuclease may comprise at least about 50, at least about 100, at least about 150, at least about 200, at least about 250, at least about 300, at least about 350, at least about 400, at least about 450, at least about 500 consecutive amino acids of any one of SEQ ID NOs: 1-34 or 87-125.

TABLE 1
Example Protein Sequences

| SEQ ID | SEQUENCE |
|---|---|
| SEQ ID NO: | MADLSQFTHKYQVPKTLRFELIPQGKTLENLSAYGMVADDKQRSENYK |
| 1 | KLKPVIDRIYKYFIEESLKNTNLDWNPLYEAIREYRKEKTTATITNLKEQQ |
| | DICRRAIASRFEGKVPDKGDKSVKDFNKKQSKLFKELFGKELFTDSVLEQ |
| | LPGVSLSDEDKALLKSFDKFTTYFVGFYDNRKNVFSSDDISTGIPHRLVQ |
| | ENFPKFIDNCDDYKRLVLVAPELKEKLEKAAEATKIFEDVSLDEIFSIKFY |
| | NRLLQQNQIDQFNQLLGGIAGAPGTPKIQGLNETLNLSMQQDKTLEQKL |
| | KSVPHRFSPLYKQILSDRSSLSFIPESFSCDAEVLLAVQEYLDNLKTEHVIE |
| | DLKEVFNRLTTLDLKHIYVNSTKVTAFSQALFGDWNLCREQLRVYKMS |
| | NGNEKITKKALGELESWLKNSDIAFTELQEALADEALPAKVNLKVQEAIS |
| | GLNEQMAKSLPKELKIPEEKEELKALLDAIQEVYHTLEWFIVSDDVETDT |
| | DFYVPLKETLQIIQPIIPLYNKVRNFATQKPYSVEKFKLNFANPTLADGW |
| | DENKEQQNCAVLFQKGNNYYLGILNPKNKPDFDNVDTEKQGNCYQKM |
| | VYKQFPDFSKMMPKCTTQLKEVKQHFEGKDSDYILNNKNFIKPLTITREV |
| | YDLNNVLYDGKKKFQIDYLRKTKDEDGYYTALHTWIDFAKKFVASYKS |
| | TSIYDTSTILPPEKYEKLNEFYGALDNLFYQIKFENIPEEIIDTYVEDGKLFL |
| | FQIYNKDFAAGATGAPNLHTIYWKAVFDPENVKDVVVKLNGQAELFYR |
| | PKSNMDVIRHKVGEKLVNRTLKDGSILTDELHKELYLYANGSLKKGLSE |
| | DAKIILDKNLAVIYDVHHEIVKDRRFTTDKFFFHVPLTLNYKCDKNPVKF |
| | NAEVQEYLKENPDTYVIGIDRGERNLIYAVVIDPKGRIVEQKSFNVINGF |
| | DYHGKLDQREKERVKARQAWTAVGKIKELKQGYLSLVVHEISKMMVR |
| | YQAVVVLENLNVGFKRVRSGIAEKAVYQQFEKMLINKLNYLMFKDAGG |
| | TEPGSVLNAYQLTDRFESFAKMGLQTGFLFYIPAAFTSKIDPATGFVDPFR |
| | WGAIKTLADKREFLSGFESLKFDSTTGNFILHFDVSKNKNFQKKLEGFVP |
| | DWDIIIEANKMKTGKGATYIAGKRIEFVRDNNSQGHYEDYLPCNALAET |
| | LRQCDIPYEEGKDILPLILEKNDSKLLHSVFKVVRLTLQMRNSNAETGED |
| | YISSPVEDVSGSCFDSRMENEKLPKDADANGAYHIALKGMLALERLRKD |
| | EKMAISNNDWLNYIQEKRA* |
| SEQ ID NO: | MAGKKKDKDVINKTLSVRIIRPRYSDDIEKEISDEKAKRKQDGKTGELDR |
| 2 | AFFSELKSRNPDIITNDELFPLFTEIQKNLTEIYNKSISLLYMKLIVEEEGGS |
| | TASALSAGPYKECKARFNSYISLGLRQKIQSNFRRKELKGFQVSLPTAKS |
| | DRFPIPFCHQVENGKGGFKVYETGDDFIFEVPLIKYTATNKKSTSGKNYT |
| | KVQLNNPPVPMNVPLLLSTMRRRQTKKGMQWNKDEGTNAELRRVMSG |
| | EYKVSYAEIIRRTRFGKHDDWFVNFSIKFKNKTDELNQNVRGGIDIGVSN |
| | PLVCAVTNGLDRYIVANNDIMAFNERAMARRRTLLRKNRFKRSGHGAK |
| | NKLEPITVLTEKNERFRKSILQRWAREVAEFFKRTSASVVNMEDLSGITE |
| | REDFFSTKLRTTWNYRLMQTTIENKLKEYGIAVNYISPKYTSQTCHSCGK |
| | RNDYFTFSYRSENNYPPFECKECNKVKCNADFNAAKNIALKVVL |
| SEQ ID NO: | MAKNTITKTLKLRIVRPYNSAEVEKIVADEKNNREKIALEKNKDKVKEA |
| 3 | CSKHLKVAAYCTTQVERNACLFCKARKLDDKFYQKLRGQFPDAVFWQ |
| | EISEIFRQLQKQAAEIYNQSLIELYYEIFIKGKGIANASSVEHYLSDVCYTR |
| | AAELFKNAAIASGLRSKIKSNFRLKELKNMKSGLPTTKSDNFPIPLVKQK |
| | GGQYTGFEISNHNSDFIIKIPFGRWQVKKEIDKYRPWEKFDFEQVQKSPK |
| | PISLLLSTQRRKRNKGWSKDEGTEAEIKKVMNGDYQTSYIEVKRGSKIGE |
| | KSAWMLNLSIDVPKIDKGVDPSIIGGIDVGVKSPLVCAINNAFSRYSISDN |
| | DLFHFNKKMFARRRILLKKNRHKRAGHGAKNKLKPITILTEKSERFRKK |
| | LIERWACEIADFFIKNKVGTVQMENLESMKRKEDSYFNIRLRGFWPYAE |
| | MQNKIEFKLKQYGIEIRKVAPNNTSKTCSKCGHLNNYFNFEYRKKNKFP |
| | HFKCEKCNFKENADYNAALNISNPKLKSTKEEP |
| SEQ ID NO: | MATLVSFTKQYQVQKTLRFELIPQGKTQANIDAKGFINDDLKRDENYMK |
| 4 | VKGVIDELHKNFIEQTLVNVDYDWRSLATAIKNYRKDRSDTNKKNLEKT |
| | QEAARKEIIAWFEGKRGNSAFKNNQKSFYGKLFKKELFSEILRSDDLEYD |
| | EETQDAIACFDKFTTYFVGFHENRKNMYSTEAKSTSVAYRVVNENFSKF |
| | LSNCEAFSVLEAVCPNVLVEAEQELHLHKAFSDLKLSDVFKVEAYNKYL |
| | SQTGIDYYNQIIGGISSAEGVRKIRGVNEVVNNAIQQNDELKVALRNKQF |
| | TMVQLFKQILSDRSTLSFVSEQFTSDQEVITVVKQFNDDIVNNKVLAVVK |
| | TLFENFNSYDLEKIYINSKELASVSNALLKDWSKIRNAVLENKIIELGANP |
| | PKTKISAVEKEVKNKDFSIAELASYNDKYLDKEGNDKEICSIANVVLEAV |
| | GALEIMLAESLPADLKTLENKNKVKGILDAYENLLHLLNYFKVSAVNDV |
| | DLAFYGAFEKVYVDISGVMPLYNKVRNYATKKPYSVEKFKLNFAMPTL |
| | ADGWDKNKERDNGSIILLKDGQYYLGVMNPQNKPVIDNAVCNDAKGY |
| | QKMVYKMFPEISKMVTKCSTQLNAVKAHFEDNTNDFVLDDTDKFISDL |
| | TITKEIYDLNNVLYDGKKKFQIDYLRNTGDFAGYHKALETWIDFVKEFLS |
| | KYRSTAIYDLTTLLPTNYYEKLDVFYSDVNNLCYKIDYENISVEQVNEW |
| | VEEGNLYLFKIYNKDFATGSTGKPNLHTMYWNAVFAEENLHDVVVKLN |
| | GGAELFYRPKSNMPKVEHRVGEKLVNRKNVNGEPIADSVHKEIYAYAN |
| | GKISKSELSENAQEELPLAIIKDVKHNITKDKRYLSDKYFFHVPITLNYKA |
| | NGNPSAFNTKVQAFLKNNPDVNIIGIDRGERNLLYVVVIDQQGNIIDKKQ |
| | VSYNKVNGYDYYEKLNQREKERIEARQSWGAVGKIKELKEGYLSLVVR |
| | EIADMMVKYNAIVVMENLNAGFKRVRGGIAEKAVYQKFEKMLIDKLNY |
| | LVFKDVEAKEAGGVLNAYQLTDKFDSFEKMGNQSGFLFYVPAAYTSKI |
| | DPVTGFANVFSTKHITNTEAKKEFICSFNSLRYDEAKDKFVLECDLNKFK |
| | IVANSHIKNWKFIIGGKRIVYNSKNKTYMEKYPCEDLKATLNASGIDFSS |
| | SEIINLLKNVPANREYGKLFDETYWAIMNTLQMRNSNALTGEDYIISAVA |
| | DDNEKVFDSRTCGAELPKDADANGAYHIALKGLYLLQRIDISEEGEKVD |
| | LSIKNEEWFKFVQQKEYAR* |
| SEQ ID NO: | MCMKITKIDGISHKKYKEKGKLIKNNDTAKDIIEERFNDIEKKTKELFQK |
| 5 | TLDFYVKNYEKCKEQNKERREKAKNYFSKVKILVDNKKITICNENTEKM |
| | EIEDFNEYDVRSGKYFNVLNKILNGENYTEEDLEVFENDLQKRTGRIKSI |
| | KNSLEENKAHFKKESINNNIIYDRVKGNNKKSLFYEYYRISSKHQEYVNN |
| | IFEAFDKLYSNSHEAMNNLFSEITKDSKDRNIRKIREAYHEILNKNKTEFG |
| | EELYKKIQDNRNNFDKLLEIEPEIKELTKSQIFYKYYIDKVNLDETSIKHCF |
| | CHLVEIEVNQLLKNYVYSKRNINKEKLENIFEYCKLKNLIKNKLVNKLN |
| | NYIRNCGKYNAYISNNDVVVNSEKISEIRTKEAFLRSIIGVSSSAYFSLRNI |
| | LNTDNTQDITNKVDKEVDKLYQENKKIELEERLKLFFGNYFDINNQQEIK |
| | VFLMNIDKIISSIRHEIIHFKMETNAQNIFDFNNVNLGNTAKNIFSNEINEE |
| | KIKFKIFKQLNSANVFDYLSNKDITEYMDKVVFSFTNRNVSFVPSFTKIYN |
| | RVQDLANSLEIKKWKIPDKSEGKDAQIYLLKNIYYGKFLDEFLNEENGIFI |
| | SIKDKIIELNRNQNKRTGFYKLEKFEKIEETNPKKYLEIIQSLYMINIEEIDS |
| | EGKNIFLDFIQKIFLKGFFEFIKNNYNYLLELKKIQDKKNIFDSEMSEYIAG |
| | EKTLEDIGEINEIIQDIKITEIDKILNQTDKINCFYLLLKLLNYKEITELKGN |
| | LEKYQILSKTNVYEKELMLLNIVNLDNNKVKIENFKILAEEIGKFIEKINIE |
| | EINKNKKIKTFEELRNFEKGENTGEYYNIYSDDKNIKNIRNLYNIKKYGM |
| | LDLLEKISEKTNYCIKKKDLEEYSELRKQLEDEKTNFYKIQEYLHSKYQQ |
| | KPKKILLKNNKNDYEKYKKSIENIEKYVHLKNKIEFNELNLLQSLLLKILH |
| | RLVGFTSIWERDLRFRLIGEFPDELDVEDIFDHRKRYKGTGKGICKKYDR |
| | FINTHTEYKNNNKMENVKFADNNPVRNYIAHFNYLPNPKYSILKMMEK |
| | LRKLLDYDRKLKNAVMKSIKDILEEYGFKAEFIINSDKEIILNLVKSVEIIH |
| | LGKEDLKSRRNSEDLCKLVKAMLEYSK* |
| SEQ ID NO: | MEDKQFLERYKEFIGLNSLSKTLRNSLIPVGSTLKHIQEYGILEEDSLRAQ |
| 6 | KREELKGIMDDYYRNYIEMHLRDVHDIDWNELFEALTEVKKNQTDDAK |
| | KRLEKIQEKKRKEIYQYLSDDAVFSEMFKEKMISGILPDFIRCNEGYSEEE |
| | KEEKLKTVALFHRFTSSFNDFFLNRKNVFTKEAIVTAIGYRVVHENAEIFL |
| | ENMVAFQNIQKSAESQISIIERKNEHYFMEWKLSHIFTADYYMMLMTQK |
| | AIEHYNEMCGVVNQQMREYCQKEKKNWNLYRMKRLHKQILSNASTSF |
| | KIPEKYENDAEVYESVNSFLQNVMEKTVMERIAVLKNSTDNFDLSKIYIT |
| | APYYEKISNYLCGSWNTITDCLTHYYEQQIAGKGARKDQKVKAAVKAD |
| | KWKSLSEIEQLLKEYARAEEVKRKPEEYIAEIENIVSLKEAHLLEYHPEVN |
| | LIENEKYATEIKDVLDNYMELFHWMKWFYIEEAVEKEVNFYGELDDLY |
| | EEIKDIVPLYNKVRNYVTQKPYSDTKIKLNFGTPTLANGWSKSKEYDYN |
| | AILLQKDGKYYMGIFNPIQKPEKEIIEGHSQPLEGNEYKKMVYYYLPSAN |
| | KMLPKVLLSKKGMEIYQPSEYIINGYKERRHIKSEEKFDLQFCHDLIDYF |
| | KSGIERNSDWKVFGFDFSDTDTYQDISGFYREVEDQGYKIDWTYIKEADI |
| | DRLNEEGKLYLFQIYNKDFSEKSTGRENLHTMYLKNLFSEENVREQVLK |
| | LNGEAEIFFRKSSVKKPIIHKKGTMLVNRTYMEEVNGNSVRRNIPEKEYQ |
| | EIYNYKNHRLKGELSTEAKKYLEKAVCHETKKDIVKDYRYSVDKFFIHL |
| | PITINYRASGKETLNSVAQRYIAHQNDMHVIGIDRGERNLIYVSVINMQG |
| | EIKEQKSFNIINEFNYKEKLKEREQSRGAARRNWKEIGQIKDLKEGYLSG |
| | VIHEIAKMMIKYHAIIAMEDLNYGFKRGRFKVERQVYQKFENMLIQKLN |
| | YLVFKDRPADEDGGVLRGYQLAYIPDSVKKMGRQCGMIFYVPAAFTSKI |
| | DPTTGFVDIFKHKVYTTEQAKREFILSFDEICYDVERQLFRFTFDYANFVT |
| | QNVTLARNNWTIYTNGTRAQKEFGNGRMRDKEDYNPKDKMVELLESE |
| | GIEFKSGKNLLPALKKVSNAKVFEELQKIVRFTVQLRNSKSEENDVDYD |
| | HVISPVLNEEGNFFDSSKYKNKEEKKESLLPVDADANGAYCIALKGLYI |
| | MQAIQKNWSEEKALSPDVLRLNNNDWFDYIQNKRYR* |
| SEQ ID NO: | MEEKKMSKIEKFIGKYKISKTLRFRAVPVGKTQDNIEKKGILEKDKKRSE |
| 7 | DYEKVKAYLDSLHRDFIENTLKKVKLNELNEYACLFFSGTKDDGDKKK |
| | MEKLEEKMRKTISNEFCNDEMYKKIFSEKILSENNEEDVSDIVSSYKGFFT |
| | SLNGYVNNRKNLYVSDAKPTSIAYRCINENLPKFLRNVECYKKVVQVIP |
| | KEQIEYMSNNLNLSPYRIEDCFNIDFFEFCLSQGGIDLYNTFIGGYSKKDG |
| | TKVQGINEIVNLYNQKNKKDKEKYKLPQFTPLFKQILSDRDTKSFSIEKLE |
| | NIYEVVELVKKSYSDEMFDDIETVFSNLNYYDASGIYVKNGPAITHISMN |
| | LTKDWATIRNNWNYEYDEKHSTKKNKNIEKYEDTRNTMYKKIDSFTLE |
| | YISRLVGKDIDELVKYFENEVANFVMDIKKTYSKLTPLFDRCQKENFDIS |
| | EDEVNDIKGYLDNVKLLESFMKSFTINGKENNIDYVFYGKFTDDYDKLH |
| | EFDHIYNKVRNYITTSRKPYKLDKYKLYFDNPQLLGGWDINKEKDYRTV |
| | MLTKDGKYYFAIIDKGEHPFDNIPKDYFDNNGYYKKIIYRQIPNAAKYLS |
| | SKQIVPQNPPEEVKRILDKKKADSKSLTEEEKNIFIDYIKSDFLKNYKLLF |
| | DKNNNPYFNFAFRESSTYESLNEFFEDVERQAYSVRYENLPADYIDNLV |
| | NEGKIYLFEIYSKDFSEYSKGTNNLHTMYFKALFDNDNLKNTVFKLSGN |
| | AELFIRPASIKKDELVIHPKNQLLQNKNPLNPKKQSIFDYDLVKDKRFFEN |
| | QYMLHISIEINKNERDAKKIKNINEMVRKELKDSDDNYIIGIDRGERNLLY |
| | VCVINSAGKIVEQMSLNEIINEYNGIKHTVDYQGLLDKCEKERNAQRQS |
| | WKSIENIKELKDGYISQVVHKLCQLVEKYDAIIAMENLNGGFKRGRTKF |
| | EKQVYQKFENKLINKMEYMADKKRKTTENGGILRGYQLTNGCINNSYQ |
| | NGFIFYVPAWLTSKIDPTTGFVDLLKPKYTNVEEAHLWINKFNSITYDKK |
| | LDMFAFNINYSQFPRADIDYRKIWTFYTNGYRIETFRNSEKNNEFDWKEV |
| | HLTSVIKKLLEEYQINYISGKNIIDDLIQIKDKPFWNSFIKYIRLTLQMRNSI |
| | TGRTDVDYIISPVINNEGTFYDSRKDLDEITLPQDADANGAYNIARKALW |
| | IIEKLKESPDEELNKVKLAITQREWLEYAQINI* |
| SEQ ID NO: | MEKIKKPSNRNSIPSIIISDYDANKIKEIKVKYLKLARLDKITIQDMEIVDNI |
| 8 | VEFKKILLNGVEHTIIDNQKIEFDNYEITGCIKPSNKRRDGRISQAKYVVTI |
| | TDKYLRENEKEKRFKSTERELPNNTLLSRYKQISGFDTLTSKDIYKIKRYI |
| | DFKNEMLFYFQFIEEFFNPLLPKGKNFYDLNIEQNKDKVAKFIVYRLNDD |
| | FKNKSLNSYITDTCMIINDFKKIQKILSDFRHALAHFDFDFIQKFFDDQLD |
| | KNKFDINTISLIETLLDQKEEKNYQEKNNYIDDNDILTIFDEKGSKFSKLH |
| | NFYTKISQKKPAFNKLINSFLSQDGVPNEEFKSYLVTKKLDFFEDIHSNKE |
| | YKKIYIQHKNLVIKKQKEESQEKPDGQKLKNYNDELQKLKDEMNTITKQ |
| | NSLNRLEVKLRLAFGFIANEYNYNFKNFNDEFTNDVKNEQKIKAFKNSS |
| | NEKLKEYFESTFIEKRFFHFSVNFFNKKTKKEETKQKNIFNSIENETLEEL |
| | VKESPLLQIITLLYLFIPRELQGEFVGFILKIYHHTKNITSDTKEDEISIEDA |
| | QNSFSLKFKILAKNLRGLQLFHYSLSHNTLYNNKQCFFYEKGNRWQSVY |
| | KSFQISHNQDEFDIHLVIPVIKYYINLNKLMGDFEIYALLKYADKNSITVK |
| | LSDITSRDDLKYNGHYNFATLLFKTFGIDTNYKQNKVSIQNIKKTRNNLA |
| | HQNIENMLKAFENSEIFAQREEIVNYLQTEHRMQEVLHYNPINDFTMKT |
| | VQYLKSLSVHSQKEGKIADIHKKESLVPNDYYLIYKLKAIELLKQKVIEVI |
| | GESEDEKKIKNAIAKEEQIKKGNN |
| SEQ ID NO: | MEKSLNDFIGLYSVSKTLRFELKPVSETLENIKKFHFLEEDKKKANDYKD |
| 87 | VKKIIDNYHKYFIDDVLKNASFNWKKLEEAIREYNKNKSDDSALVAEQK |
| | KLGDAILKLFTSDKRYKALTAATPKELFESILPDWFGEQCNQDLNKAAL |
| | KTFQKFTSYFTGFQENRKNVYSAEAIPTAVPYRIVNDNFPKFLQNVLIFKT |
| | IQEKCPQIIDEVEKELSSYLGKEKLAGIFTLESFNKYLGQGGKENQRGIDF |
| | YNQIIGGVVEKEGGINLRGVNQFLNLYWQQHPDFTKEDRRIKMVPLYKQ |
| | ILSDRSSLSFKIESIENDEELKNALLECADKLELKNDEKKSIFEEVCDLFSS |
| | VKNLDLSGIYINRKDINSVSRILTGDWSWLQSRMNVYAEEKFTTKAEKA |
| | RWQKSLDDEGENKSKGFYSLTDLNEVLEYSSENVAETDIRITDYFEHRCR |
| | YYVDKETEMFVQGSELVALSLQEMCDDILKKRKAMNTVLENLSSENKL |
| | REKTDDVAVIKEYLDAVQELLHRIKPLKVNGVGDSTFYSVYDSIYSALSE |
| | VISVYNKTRNYITKKAASPEKYKLNFDNPTLADGWDLNKEQANTSVILR |
| | KDGMFYLGIMNPKNKPKFAEKYDCGNESCYEKMIYKQFDATKQIPKCST |
| | QKKEVQKYFLSGATEPYILNDKKSFKSELIITKDIWFMNNHVWDGEKFV |
| | PKRDNETRPKKFQIGYFKQTGDFDGYKNALSNWISFCKNFLQSYLSATV |
| | YDYNFKNSEEYEGLDEFYNYLNATCYKLNFINIPETEINKMVSEGKLYLF |
| | QIYNKDFASGSTGMPNMHTLYWKNLFSDENLKNVCLKLNGEAELFYRP |
| | AGIKEPVIHKEGSYLVNRTTEDGESIPEKIYFEIYKNANGKLEKLSDEAQN |
| | YISNHEVVIKKAGHEIIKDRHYTEPKFLFHVPLTINFKASGNSYSINENVR |
| | KFLKNNPDVNIIGLDRGERHLIYLSLINQKGEIIKQFTFNEVERNKNGRTIK |
| | VNYHEKLDQREKERDAARKSWQAIGKIAELKEGYLSAVIHQLTKLMVE |
| | YNAVVVMEDLNFGFKRGRFHVEKQVYQKFEHILIDKSNYLVFKDRGLN |
| | EPGGVLNGYQIAGQFESFQKLGKQSGMLFYVPAGYTSKIDPKTGFVSMM |
| | NFKDLTNVHKKRDFFSKFDNIHYDEANGSFVFTFDYKKFDGKAKEEMK |
| | LTKWSVYSRDKRIVYFAKTKSYEDVLPTEKLQKIFESNGIDYKSGNNIQD |
| | SVMAIGADLKEGAKPSKEISDFWDGLLSNFKLILQMRNSNARTGEDYIIS |
| | PVMADDGTFFDSREEFKKGEDAKLPLDADANGAYHIALKGLSLINKINL |
| | SKDEELKKFDMKISNADWFKFAQEKNYAK* |
| SEQ ID NO: | MENYGGFTGLYPLQKTLKFELRPQGRTMEHLVSSNFFEEDRDRAEKYKI |
| 9 | VKKVIDNYHKDFINECLSKRSFDWTPLMKTSEKYYASKEKNGKKKQDL |
| | DQKIIPTIENLSEKDRKELELEQKRMRKEIVSVFKEDKRFKYLFSEKLFSIL |
| | LKDEDYSKEKLTEKEILALKSFNKFSGYFIGLHKNRANFYSEGDESTAIA |
| | YRIVNENFPKFLSNLKKYREVCEKYPEIIQDAEQSLAGLNIKMDDIFPME |
| | NFNKVMTQDGIDLYNLAIGGKAQALGEKQKGLNEFLNEVNQSYKKGND |
| | RIRMTPLFKQILSERTSYSYILDAFDDNSQLITSINGFFTEVEKDKEGNTFD |
| | RAVGLIASYMKYDLSRVYIRKADLNKVSMEIFGSWERLGGLLRIFKSELY |
| | GDVNAEKTSKKVDKWLNSGEFSLSDVINAIAGSKSAETFDEYILKMRVA |
| | RGEIDNALEKIKCINGNFSEDENSKMIIKAILDSVQRLFHLFSSFQVRADFS |
| | QDGDFYAEYNEIYEKLFAIVPLYNRVRNYLTKNNLSMKKIKLNFKNPAL |
| | ANGWDLNKEYDNTAVIFLREGKYYLGIMNPSKKKNIKFEEGSGTGPFYK |
| | KMAYKLLPDPNKMLPKVFFAKKNINYYNPSDEIVKGYKAGKYKKGENF |
| | DIDFCHKLIDFFKESIQKNEDWRAFNYLFSATESYKDISDFYSEVEDQGY |
| | RMYFLNVPVANIDEYVEKGDLFLFQIYNKDFASGAKGNKDMHTIYWNA |
| | AFSDENLRNVVVKLNGEAELFYRDKSIIEPICHKKGEMLVNRTCFDKTPV |
| | PDKIHKELFDYHNGRAKTLSIEAKGYLDRVGVFQASYEIIKDRRYSENK |
| | MYFHVPLKLNFKADGKKNLNKMVIEKFLSDKDVHIIGIDRGERNLLYYS |
| | VIDRRGNIIDQDSLNIIDGFDYQKKLGQREIERREARQSWNSIGKIKDLKE |
| | GYLSKAVHKVSKMVLEYNAIVVLEDLNFGFKRGRFKVEKQVYQKFEK |
| | MLIDKLNYLVFKEVLDSRDAGGVLNAYQLTTQLESFNKLGKQSGILFYV |
| | PAAYTSKIDPTTGFVSLFNTSRIESDSEKKDFLSGFDSIVYSAKDGGIFAFK |
| | FDYRNRNFQREKTDHKNIWTVYTNGDRIKYKGRMKGYEITSPTKRIKDV |
| | LSSSGIRYDDGQELRDSIIQSGNKVLINEVYNSFIDTLQMRNSDGEQDYIIS |
| | PVKNRNGEFFRTDPDRRELPVDADANGAYHIALRGELLMQKIAEDFDPK |
| | SDKFTMPKMEHKDWFEFMQTRGD* |
| SEQ ID NO: | MEVQKTVMKTLSLRILRPLYSQEIEKEIKEEKERRKQAGGTGELDGGFY |
| 10 | KKLEKKHSEMFSFDRLNLLLNQLQREIAKVYNHAISELYIATIAQGNKSN |
| | KHYISSIVYNRAYGYFYNAYIALGICSKVEANFRSNELLTQQSALPTAKS |
| | DNFPIVLHKQKGAEGEDGGFRISTEGSDLIFEIPIPFYEYNGENRKEPYKW |
| | VKKGGQKPVLKLILSTFRRQRNKGWAKDEGTDAEIRKVTEGKYQVSQIE |
| | INRGKKLGEHQKWFANFSIEQPIYERKPNRSIVGGLDVGIRSPLVCAINNS |
| | FSRYSVDSNDVFKFSKQVFAFRRRLLSKNSLKRKGHGAAHKLEPITEMT |
| | EKNDKFRKKIIERWAKEVTNFFVKNQVGIVQIEDLSTMKDREDHFFNQY |
| | LRGFWPYYQMQTLIENKLKEYGIEVKRVQAKYTSQLCSNPNCRYWNNY |
| | FNFEYRKVNKFPKFKCEKCNLEISADYNAARNLSTPDIEKFVAKATKGIN |
| | LPEK* |
| SEQ ID NO: | MIIHNCYIGGSFMKKIDSFTNCYSLSKTLRFKLIPIGATQSNFDLNKMLDE |
| 11 | DKKRAENYSKAKSIIDKYHRFFIDKVLSSVTENKAFDSFLEDVRAYAELY |
| | YRSNKDDSDKASMKTLESKMRKFIALALQSDEGFKDLFGQNLIKKTLPE |
| | FLESDTDKEIIAEFDGFSTYFTGFFNNRKNMYSADDQPTAISYRCINDNLP |
| | KFLDNVRTFKNSDVASILNDNLKILNEDFDGIYGTSAEDVFNVDYFPFVL |
| | SQKGIEAYNSILGGYTNSDGSKIKGLNEYINLYNQKNENIHRIPKMKQLF |
| | KQILSERESVSFIPEKFDSDDDVLSSINDYYLERDGGKVLSIEKTVEKIEKL |
| | FSAVTDYSTDGIFVKNAAELTAVCSGAFGYWGTVQNAWNNEYDALNG |
| | YKETEKYIDKRKKAYKSIESFSLADIQKYADVSESSETNAEVTEWLRNEI |
| | KEKCNLAVQGYESSKDLISKPYTESKKLFNNDNAVELIKNALDSVKELE |
| | NVLRLLLGTGKEESKDENFYGEFLPCYERICEVDSLYDKVRNYMTQKLY |
| | KTDKIKLNFQNPQFLGGWDRNKEADYSAVLLRRNSLYYIAIMPSGYKRV |
| | FEKIPAPKADETVYEKVIYKLLPGPNKMLPKVFFSKKGIETFNPPKEILEK |
| | YELGTHKTGDGFNLDDCHALIDYFKSALDVHSDWSNFGFRFSDTSTYKN |
| | IADFYNEVKNQGYKITFCDVPQSYINELVDEGKLYLFQLYNKDFSEHSKG |
| | TPNLHTLYFKMLFDERNLENVVFKLNGEAEMFYREASISKDDMIVHPKN |
| | QPIKNKNEQNSRKQSTFEYDIVKDRRYTVDQFMLHIPITLNFTANGGTNI |
| | NNEVRKALKDCDKNYVIGIDRGERNLLYICVVDSEGRIIEQYSLNEIINEY |
| | NGNTYSTDYHALLDKKEKERLESRKAWKTVENIKELKEGYISQVVHKIC |
| | ELVEKYDAVIVMEDLNLGFKQGRSGKFEKSVYQKFEKMLIDKLNYFAD |
| | KKKSPEEIGSVLNAYQLTNAFESFEKMGKQNGFIFYVPAYLTSKIDPTTG |
| | FADLLHPSSKQSKESMRDFVGRFDSITFNKTENYFEFELDYNKFPRCNTD |
| | YRKKWTVCTYGSRIKTFRNPEKNSEWDNKTVELTPAFMALFEKYSIDVN |
| | GDIKAQIMSVDKKDFFVELIGLLRLTLQMRNSETGKVDRDYLISPVKNSE |
| | GVFYNSDDYKGIENASLPKDADANGAYNIARKGLWIIEQIKACENDAEL |
| | NKIRLAISNAEWLEYAQKK* |
| SEQ ID NO: | MKDYIRKTLSLRILRPYYGEEIEKEIAAAKKKSQAEGGDGALDNKFWDR |
| 12 | LKAEHPEIISSREFYDLLDAIQRETTLYYNRAISKLYHSLIVEREQVSTAKA |
| | LSAGPYHEFREKFNAYISLGLREKIQSNFRRKELARYQVALPTAKSDTFPI |
| | PIYKGFDKNGKGGFKVREIENGDFVIDLPLMAYHRVGGKAGREYIELDR |
| | PPAVLNVPVILSTSRRRANKTWFRDEGTDAEIRRVMAGEYKVSWVEILQ |
| | RKRFGKPYGGWYVNFTIKYQPRDYGLDPKVKGGIDIGLSSPLVCAVTNS |
| | LARLTIRDNDLVAFNRKAMARRRTLLRQNRYKRSGHGSANKLKPIEALT |
| | EKNELYRKAIMRRWAREAADFFRQHRAATVNMEDLTGIKDREDYFSQM |
| | LRCYWNYSQLQTMLENKLKEYGIAVKYIEPKDTSKTCHSCGHVNEYFDF |
| | NYRSAHKFPMFKCEKCGVECGADYNAARNIAQA |
| SEQ ID NO: | MKEQFINRYPLSKTLRFSLIPVGETENNFNKNLLLKKDKQRAENYEKVK |
| 13 | CYIDRFHKEYIESVLSKARIEKVNEYANLYWKSNKDDSDIKAMESLEND |
| | MRKQISKQLTSTEIYKKRLFGKELICEDLPSFLTDKDERETVECFRSFTTY |
| | FKGFNTNRENMYSSDGKSTAIAYRCINDNLPRFLDNVKSFQKVFDNLSD |
| | ETITKLNTDLYNIFGRNIEDIFSVDYFEFVLTQSGIEIYNSMIGGYTCSDKT |
| | KIQGLNECINLYNQQVAKNEKSKKLPLMKPLYKQILSEKDSVSFIPEKFN |
| | SDNEVLHAIDDYYTGHIGDFDLLTELLQSLNTYNANGIFVKSGVAITDISN |
| | GAFNSWNVLRSAWNEKYEALHPVTSKTKIDKYIEKQDKIYKAIKSFSLFE |
| | LQSLGNENGNEITDWYISSINESNSKIKEAYLQAQKLLNSDYEKSYNKRL |
| | YKNEKATELVKNLLDAIKEFQKLIKPLNGTGKEENKDELFYGKFTSYYD |
| | SIADIDRLYDKVRNYITQKPYSKDKIKLNFDNPQLLGGWDKNKESDYRT |
| | VLLHKDGLYYLAVMDKSHSKAFVDAPEITSDDKDYYEKMEYKLLPGPN |
| | KMLPKVFFASKNIDTFQPSDRILDIRKRESFKKGATFNKAECHEFIDYFKD |
| | SIKKHDDWSQFGFKFSPTESYNDISEFYREISDQGYSVRFNKISKNYIDGL |
| | VNNGYIYLFQIYNKDFSKYSKGTPNLHTLYFKMLFDERNLSNVVYKLNG |
| | EAEMFYREASIGDKEKITHYANQPIKNKNPDNEKKESVFEYDIVKDKRFT |
| | KRQFSLHLPITINFKAHGQEFLNYDVRKAVKYKDDNYVIGIDRGERNLIY |
| | ISVINSNGEIVEQMSLNEIISDNGHKVDYQKLLDTKEKERDKARKNWTSV |
| | ENIKELKEGYISQVVHKICELVIKYDAVIAMEDLNFGFKRGRFPVEKQVY |
| | QKFENMLISKLNLLIDKKAEPTEDGGLLRAYQLTNKFDGVNKAKQNGIIF |
| | YVPAWDTSKIDPATGFVNLLKPKCNTSVPEAKKLFETIDDIKYNANTDM |
| | FEFYIDYSKFPRCNSDFKKSWTVCTNSSRILTFRNKEKNNKWDNKQIVLT |
| | DEFKSLFNEFGIDYKGNLKDSILSISNADFYRRLIKLLSLTLQMRNSITGST |
| | LPEDDYLISPVANKSGEFYDSRNYKGTNAALPCDADANGAYNIARKAL |
| | WAINVLKDTPDDMLNKAKLSITNAEWLEYTQK* |
| SEQ ID NO: | MKEQFVNQYPISKTLRFSLIPIGKTEENFNKNLLLKEDEKKAEEYQKVKG |
| 14 | YIDRYHKFFIETALCNINFEGFEEYSLLYYKCSKDDNDLKTMEDIEIKLRK |
| | QISKTMTSHKLYKDLFGENMIKTILPNFLDSDEEKNSLEMFRGFYTYFSG |
| | FNTNRKNMYTEEAKSTSIAYRCINDNLPKFLDNSKSFEKIKCALNKEELK |
| | AKNEEFYEIFQIYATDIFNIDFFNFVLTQPGIDKYNGIIGGYTCSDGTKVQG |
| | LNEIINLYNQQIAKDDKSKRLPLLKMLYKQILSDRETVSFIPEKFSSDNEV |
| | LESINNYFSKNVSNAIKSLKELFQGFEAYNMNGIFISSGVAITDLSNAVFG |
| | DWNAISTAWEKAYFETNPPKKNKSQEKYEEELKANYKKIKSFSLDEIQR |
| | LGSIAKSPDSIGSVAEYYKITVTEKIDNITELYDGSKELLNCNYSESYDKK |
| | LIKNDTVIEKVKTLLDAVKSLEKLIKPLVGTGKEDKDELFYGTFLPLYTSL |
| | SAVDRLYDKVRNYATQKPYSKDKIKLNFNCSSFLSGWATDYSSNGGLIF |
| | EKDGLYYLGIVNKKFTTEEIDYLQQNADENPAQRIVYDFQKPDNKNTPR |
| | LFIRSKGTNYSPSVKEYNLPVEEIVELYDKRYFTTEYRNKNPELYKASLV |
| | KLIDYFKLGFTRHESYRHYDFKWKKSEEYNDISEFYKDVEISCYSLKQEK |
| | INYNTLLNFVAENRIYLFQIYNKDFSKYSKGTPNLHTRYFKALFDENNLS |
| | DVVFKLNGGSEMFFRKASIKDNEKVVHPANQPIDNKNPDNSKKQSTFDY |
| | ELIKDKRFTKHQFSIHIPITMNFKARGRDFINNDIRKAIKSEYKPYVIGIDR |
| | GERNLIYISVINNNGEIVEQMSLNDIISDNGYKVDYQRLLDRKEKERDNA |
| | RKSWGTIENIKELKEGYISQVIHKICELVIKYDAVIAMEDLNFGFKRGRFN |
| | VEKQVYQKFENMLISKLNYLCDKKSEANSEGGLLKAYQLTNKFDGVNK |
| | GKQNGIIFYVPAWLTSKIDPVTGFVDLLHPKYISVEETHSLFEKLDDIRYN |
| | FEKDMFEFDIDYSKLPKCNADFKQKWTVCTNADRIMTFRNSEKNNEWD |
| | NKRILLSDEFKRLFEEFGIDYCHNLKNKILSISNKDFCYRFIKLFALTMQM |
| | RNSITGSTNPEDDYLISPVRDENGVFYDSRNFIGSKAGLPIDADANGAYNI |
| | ARKGLWAINAIKSTADDMLDKVDLSISNAKWLEYVQK* |
| SEQ ID NO: | MKITKIDGILHKKYIKEGKLVKSTSEENKTDERLSELLTIRLDTYIKNPDN |
| 15 | ASEEENRIRRETLKEFFSNKVLYLKDSILYLKDRREKNQLQNKNYSEEDIS |
| | EYDLKNKNSFLVLKKILLNEDINSEELEIFRNDFEKKLDKINSLKYSLEEN |
| | KANYQKINENNIKKVEGKSKRNIFYNYYKDSAKRNDYINNIQEAFDKLY |
| | KKEDIENLFFLIENSKKHEKYKIRECYHKIIGRKNDKENFATIIYEEIQNVN |
| | NMKELIEKVPNVSELKKSQVFYKYYLNKEKLNDENIKYVFCHFVEIEMS |
| | KLLKNYVYKKPSNISNDKVKRIFEYQSLKKLIENKLLNKLDTYVRNCGK |
| | YSFYLQDGEIATSDFIVGNRQNEAFLRNIIGVSSTAYFSLRNILETENENDI |
| | TGRIKGKTVKNKKGEEKYISGEIDKLYDNNKQNEVKKNLKMFYSYDFN |
| | MNRKKEIEDFFSNIDEAISSIRHGIVHFNLELEGKDIFTFKNIVPSQISKKMF |
| | QNEINEKKLKLKIFRQLNSANVFRYLEKYKILNYLNRTRFEFVNKNIPFVP |
| | SFTKLYSRIDDLKNSLCIYWKIPKANDNNKTKEITDAQIYLLKNIYYGEFL |
| | NYFMSNNGNFFEIIKEIIELNKNDKRNLKTGFYKLQKFENLQEKTPKEYL |
| | ANIQSFYMIDAGNKDEEEKDAYIDFIQKIFLKGFMTYLANNGRLSLMYIG |
| | NDEQINTSLAGKKQEFDKFLKKYEQNNNIEIPHEINEFVREIKLGKILKYT |
| | ESLNMFYLILKLLNHKELTNLKGSLEKYQSANKEEAFSDQLELINLLNLD |
| | NNRVTEDFELEADEIGKFLDFNGNKVKDNKELKKFDTNKIYFDGENIIKH |
| | RAFYNIKKYGILNLLEKISDEAKYKISIEELKNYSNKKIEIEKNHTTQENLH |
| | RKYARPRKDEKFNDEDYKKYEKTIRNIQQYTHLKNKVEFNELNLLQSLL |
| | LRILHRLVGYTSIWERDLRFRLKGEFPENQYIEEIFNFDNSKNVKYKNGQI |
| | VEKYISFYKELYKDDMEKISIYSDKKVKELKKEKKDLYIRNYIAHFNYIP |
| | NAEVSLLEVLENLRKLLSYDRKLKNAIMKSIVDILKEYGFVVTFKIEKDK |
| | KIRIESLKSEEVVHLKKLKLKDNDKKKEPIKTYRNSKELCKLVKVMFEY |
| | KMKEKKSEN* |
| SEQ ID NO: | MKITKIDGISHKKYIKEGKLVKSTSEENKTDERLSELLTIRLDTYIKNPDN |
| 16 | ASEEENRIRRENLKEFFSNKVLYLKDGILYLKDRREKNQLQNKNYSEEDI |
| | SEYDLKNKNSFLVLKKILLNEDINSEELEIFRKDVEAKLNKINSLKYSFEE |
| | NKANYQKINENNVEKVGGKSKRNIIYDYYRESAKRNDYINNVQEAFDK |
| | LYKKEDIEKLFFLIENSKKHEKYKIRECYHKIIGRKNDKENFAKIIYEEIQN |
| | VNNIKELIEKVPDMSELKKSQVFYKYYLDKEELNDKNIKYAFCHFVEIE |
| | MSQLLKNYVYKRLSNISNDKIKRIFEYQNLKKLIENKLLNKLDTYVRNC |
| | GKYNYYLQDGEIATSDFIAGNRQNEAFLRNIIGVSSVAYFSLRNILETENK |
| | DDITGKMRGKTRIDSKTGEEKYIPGEVDQIYYENKQNEVKNKLKMFYGY |
| | DFDMDNKKEIEDFFANIDEAISSIRHGIVHFNLDLDGKDIFAFKNIVPSEIS |
| | KKMFQNEINEKKLKLKIFRQLNSANVFRYLEKYKILNYLKRTRFEFVNK |
| | NIPFVPSFTKLYSRIDDLKNSLGIYWKTPKTNDDNKTKEIIDAQIYLLKNIY |
| | YGEFLNYFMSNNGNFFEISREIIELNKNDKRNLKTGFYKLQKFEDIQEKTP |
| | KKYLANIQSLYMINAGNQDEEEKDTYIDFIQKIFLKGFMTYLANNGRLSL |
| | MYIGNDEQINTSLAGKKQEFDKFLKKYEQNNNIEIPHEINEFLREIKLGKI |
| | LKYTESLNMFYLILKLLNHKELTNLKGSLEKYQSANKEETFSDELELINL |
| | LNLDNNRVTEDFELEANEIGKFLDFNGNKIKDRKELKKFDTKKIYFDGEN |
| | IIKHRAFYNIKKYGMLNLLEKIADKAKYKISLKELKEYSNKKNEIEKNYT |
| | MQQNLHRKYARPKKDEKFNDEDYKEYEKAIGNIQKYTHLKNKVEFNEL |
| | NLLQGLLLKILHRLVGYTSIWERDLRFRLKGEFPENQYIEEIFNFDNSKNV |
| | KYKSGQIVEKYINFYKELYKDNVEKRSIYSDKKVKKLKQEKKDLYIRNY |
| | IAHFNYIPHAEISLLEVLENLRKLLSYDRKLKNAIMKSVVDILKEYGFVAT |
| | FKIGADKKIGIQTLESEKIVHLKNLKKKKLMTDRNSEELCKLVKVMFEY |
| | KMEEEKNLKTKKCKVI* |
| SEQ ID NO: | MKKIDNFVGCYPVSKTLRFKAIPIGKTQENIEKKRLVEEDEVRAKDYKA |
| 17 | VKKLIDRYHREFIEGVLDNVKLDGLEEYYMLFNKSDREESDNKKIEIMEE |
| | RFRRVISKSFKNNEEYKKIFSKKIIEEILPNYIKDEEEKELVKGFKGFYTAF |
| | VGYAQNRENMYSDEKKSTAISYRIVNENMPRFITNIKVFEKAKSILDVDK |
| | INEINEYILNNDYYVDDFFNIDFFNYVLNQKGIDIYNAIIGGIVTGDGRKIQ |
| | GLNECINLYNQENKKIRLPQFKPLYKQILSESESMSFYIDEIESDDMLIDM |
| | LKESLQIDSTINNAIDDLKVLFNNIFDYDLSGIFINNGLPITTISNDVYGQW |
| | STISDGWNERYDVLSNAKDKESEKYFEKRRKEYKKVKSFSISDLQELGG |
| | KDLSICKKINEIISEMIDDYKSKIEEIQYLFDIKELEKPLVTDLNKIELIKNSL |
| | DGLKRIERYVIPFLGTGKEQNRDEVFYGYFIKCIDAIKEIDGVYNKTRNYL |
| | TKKPYSKDKFKLYFENPQLMGGWDRNKESDYRSTLLRKNGKYYVAIID |
| | KSSSNCMMNIEEDENDNYEKINYKLLPGPNKMLPKVFFSKKNREYFAPS |
| | KEIERIYSTGTFKKDTNFVKKDCENLITFYKDSLDRHEDWSKSFDFSFKES |
| | SAYRDISEFYRDVEKQGYRVSFDLLSSNAVNTLVEEGKLYLFQLYNKDF |
| | SEKSHGIPNLHTMYFRSLFDDNNKGNIRLNGGAEMFMRRASLNKQDVT |
| | VHKANQPIKNKNLLNPKKTTTLPYDVYKDKRFTEDQYEVHIPITMNKVP |
| | NNPYKINHMVREQLVKDDNPYVIGIDRGERNLIYVVVVDGQGHIVEQLS |
| | LNEIINENNGISIRTDYHTLLDAKERERDESRKQWKQIENIKELKEGYISQ |
| | VVHKICELVEKYDAVIALEDLNSGFKNSRVKVEKQVYQKFEKMLITKLN |
| | YMVDKKKDYNKPGGVLNGYQLTTQFESFSKMGTQNGIMFYIPAWLTSK |
| | MDPTTGFVDLLKPKYKNKADAQKFFSQFDSIRYDNQEDAFVFKVNYTK |
| | FPRTDADYNKEWEIYTNGERIRVFRNPKKNNEYDYETVNVSERMKELFD |
| | SYDLLYDKGELKETICEMEESKFFEELIKLFRLTLQMRNSISGRTDVDYLI |
| | SPVKNSNGYFYNSNDYKKEGAKYPKDADANGAYNIARKVLWAIEQFK |
| | MADEDKLDKTKISIKNQEWLEYAQTHCE |
| SEQ ID NO: | MKKIDSFVNYYPLSKTLRFSLIPVGKTEDNFNAKLLLEEDEKRAIEYEKV |
| 18 | KRYIDRYHKHFIETVLANFHLDDVNEYAELYYKAGKDDKDLKYMEKLE |
| | GKMRKSISAAFTKDKKYKEIFGQEIIKNILPEFLENEDEKESVKMFQGFFT |
| | YFTGFNDNRKNMYTHEAQTTAISYRCINENLPKFLDNVQSFAKIKESISS |
| | DIMNKLDEVCMDLYGVYAQDMFCTDYFSFVLSQSGIDRYNNIIGGYVD |
| | DKGVKIQGINEYINLYNQQVDEKNKRLPLMKKLYKQILIEKESISFIPEKF |
| | ESDNIVINAISDYYHNNVENLFDDFNKLFNEFSEYDDNGIFVTSGLAVTDI |
| | SNAVFGSWNIISDSWNEEYKDSHPMKKTTNAEKYYEDMKKEYKKNLSF |
| | TIAELQRLGEAGCNDECKGDIKEYYKTTVAEKIENIKNAYEISKDLLASD |
| | YEKSNDKKLCKNDSAISLLKNLLDSIKDLEKTIKPLLGTGKEENKDDVFY |
| | GKFTNLYEMISEIDRLYDKVRNYVTQKPYSKDKIKLNFENPQHLGGWDK |
| | NKERDYRSVLLKKEDKYYLAIMDKSNNKAFIDFPDDGECYEKIEYKLLP |
| | GPNKMLPKVFFASSNIEYFAPSKKILEIRSRESFKKGDMFNLKDCHEFIDF |
| | FKESIKKHEDWSQFGFEFSPTEKYNDISEFYNEVKIQGYSLKYKNVSKKY |
| | IDELIECGQLYLFQIYNKDFSVYAKGNPNLHTMYFKMLFDERNLANVVY |
| | QLNGGAEMFYRKASIKDSEKIVHHANQPIKNKNADNVKKESVFEYDIIK |
| | DKRFTKRQFSIHIPITLNFKAKGQNFINNDVRMALKKADENYVIGIDRGE |
| | RNLLYICVINSKGEIVEQKSLNEIIGDNGYRVDYHKLLDKKEAERDEARK |
| | SWGTIENIKELKEGYLSQIVHEISKLVIKYDAVIAIEDLNSGFKKGRFKVE |
| | KQVYQKFENMLCTKLNYLVDKNADANECGGLLKAYQLTNKEDGANRG |
| | RQNGIIFSVPAWLTSKIDPVTGFADLLRPKYKSVSESVEFISKIDNIRYNSK |
| | EDYFEFDIDYSKFPNSTASYKKKWTVCTYGERIINVRNKEKNNMWDNK |
| | TIVLTDEFKKLFADFGVDVSKNIKESVLAIDSKDFYYRFINLLANTLQLRN |
| | SEVGNVDVDYLISPVKGVDGSFYDSRLVKEKTLPENADANGAYNIARKA |
| | LWAIDVLKQTKDEELKNANLSIKNAEWLEYVQK* |
| SEQ ID NO: | MKNQNTLPSNPTDILKDKPFWAAFFNLARHNVYLTVNHINKLLDLEKLY |
| 19 | NKDKHKEIFEHEDIFNISDDVMNDVNSNGKKRKLDIKKIWANLDTDLTR |
| | KYQLRELILKHFPFIQPAIIGAQTKERTTIDKDKRSTSTSNDSLKPTGEGDI |
| | NDPLSLSNVKSIFFRLLQMLEQLRNYYSHVKHSKSATMPNFDEGLLKSM |
| | YNIFIDSVNKVKEDYSSNSVIDPNTSFSHLISKDEQGEIKPCRYSFTSKDGS |
| | INASGLLFFVSLFLEKQDSIWMQKKIPGFKKTSENYMKMTNEVFCRNHIL |
| | LPKMRLETVYDKDWMLLDMLNEVVRCPLSLYKRLAPADQNKFKVPEK |
| | SSDNANRQEDDNPFSRILVRHQNRFPYFALRFFDLNEVFTTLRFQINLGC |
| | YHFAICKKQIGDKKEVHHLTRTLYGFSRLQNFTQNTRPEEWNTLVKTTE |
| | PSSGNDGKTVQGVPLPYISYTIPHYQIENEKIGIKIFDGDTAVDTDIWPSVS |
| | TEKQLNKPDKYTLTPGFKADVFLSVHELLPMMFYYQLLLCEGMLKTDA |
| | GNAVEKVLIDTRNAIFNLYDAFVQEKINTITDLENYLQDKPILIGHLPKQ |
| | MIDLLKGHQRDMLKAVEQKKAMLIKDTERRLERLNKQPEQKPNVAAK |
| | NTGTLLRNGQIADWLVKDMMRFQPVKRDKEGNPINCSKANSTEYQMLQ |
| | RAFAFYTTDSYRLPRYFEQLHLINCDNSHLFLSRFEYDKQPNLIAFYAAY |
| | LEAKLEFLNELQPQNWASDNYFLLLRAPKNDRQKLAEGWKNGFNLPRG |
| | LFTEKIKTWFNEHKTIVDISDCDIFKNRVGQVARLIPVFFDKKFKDHSQPF |
| | YTYNFNVGNVSKITEANYLSKEKRENLFKSYQNKFKNNIPAEKTKEYRE |
| | YKNFSSWKKFERELRLIKNQDILTWLMCKNLFDEKIKPKKDILEPRIAVS |
| | YIKLDSLQTNTSTAGSLNALAKVVPMTLAIHIDSPKPKGKAGNNEKENK |
| | EFTVYIKEEGTKLLKWGNFKTLLADRRIKGLFSYIEHDDINLEKYPLTKY |
| | QVDSELDLYQKYRIDIFKQTLDLEAQLLDKYSDLNTDNFNQMLSGWSEK |
| | EGIPRNIKQDVAFLIGVRNGFSHNQYPDSKRIAFSRIKKENPKTSSLQESEG |
| | LNIAKQMYEEAQQVVNKIKNIESFD* |
| SEQ ID NO: | MKVTKIDGISHKKFEDEGKLVKFTGHFNIKNEMKERLEKLKELKLSNYIK |
| 20 | NPENVKNKDKNKEKETKSRRENLKKYFSEIILRKKEEKYLLKKTRKFKNI |
| | TEEINYDDIKKRENQQKIFDVLKELLEQRINENDKEEILNFDSVKLKEAFE |
| | EDFIKKELKIKAIEESLEKNRADYRKDYVELENEKYEDVKGQNKRSLVFE |
| | YYKNPENREKFKENIKYAFENLYTEENIKNLYSEIKEIFEKVHLKSKVRYF |
| | YQNEIIGESEFSEKDEEGISILYKQIINSVEKKEKFIEFLQKVKIKDLTRSQIF |
| | YKYFLENEELNDENIKYVFSYFVEIEVNKLLKENVYKTKKFNEGNKYRV |
| | KNIFNYDKLKNLVVYKLENKLNNYVRNCGKYNYHMENGDIATSDINM |
| | KNRQTEAFLRSILGVSSFGYFSLRNILGVNDDDFYKIEKDERKNENFILKK |
| | AKEDFTSKNIFEKVVDKSFEKKGIYQIKENLKMFYGNSFDKVDKDELKK |
| | FFVNMLEAITSVRHRIVHYNINTNSENIFDFSNIEVSKLLKNIFEKEIDTRE |
| | LKLKIFRQLNSAGVFDYWESWVIKKYLENVKFEFVNKNVPFVPSFKKLY |
| | DRIDNLKGWNALKLGNNINIPKRKEAKDSQIYLLKNIYYGEFVEKFVND |
| | NKNFEKIVKEIIEINRGAGTNKKTGFYKLEKFETLKANTPTKYLEKLQSL |
| | HKISYDKEKIEEDKDVYVDFVQKIFLKGFVNYLKKLDSLKSLNLLNLRK |
| | DETITDKKSVHDEKLKLWENSGSNLSKMPEEIYEYVKKIKISNINYNDRM |
| | SIFYLLLKLIDYRELTNLRGNLEKYESMNKNKIYSEELTIINLVNLDNNKV |
| | RTNFSLEAEDIGKFLKSSITIKNIAQLNNFSKIFADGENVIKHRSFYNIKKY |
| | GILDLLEKIVAKADLKITKEEIKKYENLQNELKRNDFYKIQEQIHRNYNQ |
| | KPFSIKKIENKKDFEKYKKVIEKIQDYTQLKNKIEFNDLNLLQSLIFRILHR |
| | LAGYTSLWERDLQFKLKGEFPEDKYIDEIFNSDGNNNQKYKHGGIADKY |
| | ANFLIEKKEEKSGEILNKKQRKKKIKEDLEIRNYIAHFNYLPNAEKSILEIL |
| | EELRELLKHDRKLKNAVMKSIKDIFREYGFIVEFTISHTKNGKKIKVCSV |
| | KSEKIKHLKNNELITTRNSEDLCELVKIMLEHKELQK* |
| SEQ ID NO: | MKVTKVGGISHKKYTSEGRLVKSESEENRTDERLSALLNMRLDMYIKNP |
| 21 | SSTETKENQKRIGKLKKFFSNKMVYLKDNTLSLKNGKKENIDREYSETDI |
| | LESDVRDKKNFAVLKKIYLNENVNSEELEVFRNDIKKKLNKINSLKYSFE |
| | KNKANYQKINENNIEKVEGKSKRNIIYDYYRESAKRDAYVSNVKEAFDK |
| | LYKEEDIAKLVLEIENLTKLEKYKIREFYHEIIGRKNDKENFAKIIYEEIQN |
| | VNNMKELIEKVPDMSELKKSQVFYKYYLDKEELNDKNIKYAFCHFVEIE |
| | MSQLLKNYVYKRLSNISNDKIKRIFEYQNLKKLIENKLLNKLDTYVRNC |
| | GKYNYYLQDGEIATSDFIARNRQNEAFLRNIIGVSSVAYFSLRNILETENE |
| | NDITGRMRGKTVKNNKGEEKYVSGEVDKIYNENKKNEVKENLKMFYS |
| | YDFNMDNKNEIEDFFANIDEAISSIRHGIVHFNLELEGKDIFAFKNIAPSEIS |
| | KKMFQNEINEKKLKLKIFRQLNSANVFRYLEKYKILNYLKRTRFEFVNK |
| | NIPFVPSFTKLYSRIDDLKNSLGIYWKTPKTNDDNKTKEIIDAQIYLLKNIY |
| | YGEFLNYFMSNNGNFFEISKEIIELNKNDKRNLKTGFYKLQKFEDIQEKIP |
| | KEYLANIQSLYMINAGNQDEEEKDTYIDFIQKIFLKGFMTYLANNGRLSL |
| | IYIGSDEETNTSLAEKKQEFDKFLKKYEQNNNIKIPYEINEFLREIKLGNIL |
| | KYTERLNMFYLILKLLNHKELTNLKGSLEKYQSANKEEAFSDQLELINLL |
| | NLDNNRVTEDFELEADEIGKFLDFNGNKVKDNKELKKFDTNKIYFDGEN |
| | IIKHRAFYNIKKYGMLNLLEKIADKAGYKISIEELKKYSNKKNEIEKNHK |
| | MQENLHRKYARPRKDEKFTDEDYESYKQAIENIEEYTHLKNKVEFNELN |
| | LLQGLLLRILHRLVGYTSIWERDLRFRLKGEFPENQYIEEIFNFENKKNVK |
| | YKGGQIVEKYIKFYKELHQNDEVKINKYSSANIKVLKQEKKDLYIRNYIA |
| | HFNYIPHAEISLLEVLENLRKLLSYDRKLKNAVMKSVVDILKEYGFVATF |
| | KIGADKKIGIQTLESEKIVHLKNLKKKKLMTDRNSEELCKLVKIMFEYK |
| | MEEKKSEN |
| SEQ ID NO: | MNELVKNRCKQTKTICQKLIPIGKTRETIEKYNLMEIDRKIAANKELMNK |
| 22 | LFSLIAGKHINDTLSKCTDLDFEPLLTSLSSLNNAKENDRDNLREYYDSVF |
| | EEKKTLAEEISSRLTAVKFAGKDFFTKNIPDFLETYEGDDKNEMSELVSL |
| | VIENTVTAGYVKKLEKIDRSMEYRLVSGTVVKRVLTDNADIYEKNIEKA |
| | KDFDYGVLNIDEASQFTTLVAKDYANYLTADGIAIYNVGIGKINLALNEY |
| | CQKNKEYSYNKLALLPLQKMLYGEKLSLFEKLEDFTSDEELINSYNKFA |
| | KTVNESGLAEIIKKAVPSYDEIVIKPNKISNYSNSITGHWSLVNRIMKDYL |
| | ENNGIKNADKYMEKGLTLSEIGDALENKNIKHSDFISNLINDLGHTYTEIK |
| | ENKESLKKDESVNALIIKKELDMLLSILQNLKVFDIDNEMFDTGFGIEVSK |
| | AIEILGYGVPLYNKIRNYITKKPDPKKKFMTKFGSATIGTGITTSVEGSKK |
| | ATFLKDGDAVFLLLYNTAGCKANNVSVSNLADLINSSLEIENSGKCYQK |
| | MIYQTPGDIKKQIPRVFVYKSEDDDLIKDFKAGLHKTDLSFLNGRLIPYL |
| | KEAFATHETYKNYTFSYRNSYESYDEFCEHMSEQAYILEWKWIDKKLID |
| | DLVEDGSLLMFRVWNRFMKKKEGKISKHAKIVNELFSDENASNAAIKLL |
| | SVFDIFYRDKQIDNPIVHKAGTTLYNKRTKDGEVIVDYTTMVKNKEKRP |
| | NVYTTTKKYDIIKDRRYTEEQFEIHLHVNIGKEENKEKLETSKVINEKKN |
| | TLVVTRSNEHLLYVVIFDENDNILLKKSLNTVKGMNFKSKLEVVEIQKKE |
| | NMQSWKTVGSNQALMEGYLSFAIKEIADLVKEYDAILVLEQNSVGKNIL |
| | NERVYTRFKEMLITNLSLDVDYENKDFYSYTELGGKVASWRDCVTNGIC |
| | IQVPSAYKYKDPTTSFSTISMYAKTTAEKSKKLKQIKSFKYNRERGLFEL |
| | VIAKGVGLENNIVCDSFGSRSIIENDISKEVSCTLKIEKYLIDAGIEYNDEK |
| | EVLKDLDTAAKTDAVHKAVTLLLKCFNESPDGRYYISPCGEHFTLCDAP |
| | EVLSAINYYIRSRYIREQIVEGVKKMEYKKTILLAK* |
| SEQ ID NO: | MNGNRIIVYREFVGVTPVAKTLRNELRPIGHTQEHIIHNGLIQEDELRQEK |
| 23 | STELKNIMDDYYREYIDKSLSGVTDLDFTLLFELMNLVQSSPSKDNKKAL |
| | EKEQSKMREQICTHMQSDSNYKNIFNAKFLKEILPDFIKNYNQYDAKDK |
| | AGKLETLALFNGFSTYFTDFFEKRKNVFTKEAVSTSIAYRIVHENSLTFLA |
| | NMTSYKKISEKALDEIEVIEKNNQDKMGDWELNQIFNPDFYNMVLIQSGI |
| | DFYNEICGVVNAHMNLYCQQTKNNYNLFKMRKLHKQILAYTSTSFEVP |
| | KMFEDDMSVYNAVNAFIDETEKGNIIGKLKDIVNKYDELDEKRIYISKDF |
| | YETLSCFMSGNWNLITGCVENFYDENIHAKGKSKEEKVKKAVKEDKYK |
| | SINDVNDLVEKYIDEKERNEFKNSNAKQYIREISNIITDTETAHLEYDEHIS |
| | LIESEEKADEMKKRLDMYMNMYHWAKAFIVDEVLDRDEMFYSDIDDIY |
| | NILENIVPLYNRVRNYVTQKPYNSKKIKLNFQSPTLANGWSQSKEFDNN |
| | AIILIRDNKYYLAIFNAKNKPDKKIIQGNSDKKNDNDYKKMVYNLLPGA |
| | NKMLPKVFLSKKGIETFKPSDYIISGYNAHKHIKTSENFDISFCRDLIDYFK |
| | NSIEKHAEWRKYEFKFSATDSYNDISEFYREVEMQGYRIDWTYISEADIN |
| | KLDEEGKIYLFQIYNKDFAENSTGKENLHTMYFKNIFSEENLKDIIIKLNG |
| | QAELFYRRASVKNPVKHKKDSVLVNKTYKNQLDNGDVVRIPIPDDIYNE |
| | IYKMYNGYIKENDLSEAAKEYLDKVEVRTAQKDIVKDYRYTVDKYFIHT |
| | PITINYKVTARNNVNDMAVKYIAQNDDIHVIGIDRGERNLIYISVIDSHGN |
| | IVKQKSYNILNNYDYKKKLVEKEKTREYARKNWKSIGNIKELKEGYISG |
| | VVHEIAMLMVEYNAIIAMEDLNYGFKRGRFKVERQVYQKFESMLINKL |
| | NYFASKGKSVDEPGGLLKGYQLTYVPDNIKNLGKQCGVIFYVPAAFTSK |
| | IDPSTGFISAFNFKSISTNASRKQFFMQFDEIRYCAEKDMFSFGFDYNNFD |
| | TYNITMSKTQWTVYTNGERLQSEFNNARRTGKTKSINLTETIKLLLEDNE |
| | INYADGHDVRIDMEKMDEDKNSEFFAQLLSLYKLTVQMRNSYTEAEEQ |
| | EKGISYDKIISPVINDEGEFFDSDNYKESDDKECKMPKDADANGAYCIAL |
| | KGLYEVLKIKSEWTEDGFDRNCLKLPHAEWLDFIQNKRYE* |
| SEQ ID NO: | MNKDIRKNFTDFVGISEIQKTLRFILIPIGKTAQNIDKYNMFEDDEIRHEY |
| 24 | YPILKEACDDFYRNHIDQQFENLELDWSKLDEALASEDRDLINETRATYR |
| | QVLFNRLKNSVDIKGDSKKNKTLSLESSDKNLGKKKTKNTFQYNFNDLF |
| | KAKLIKAILPLYIEYIYEGEKLENAKKALKMYNRFTSRLSNFWQARANIF |
| | TDDEISTGSPYRLVNDNFTIFRINNSIYTKNKPFIEEDILEFEKKLKSKKIIK |
| | DFESVDDYFTVNAFNKLCTQNGIDKYNSILGGFTTKEREKVKGLNELFN |
| | LAQQSINKGKKGEYRKNIRLGKLTKLKKQILAISDSTSFLIEQIEDDQDLY |
| | NKIKDFFELLLKEEIENENIFTQYANLQKLIEQADLSKIYINAKHLNKISHQ |
| | VTGKWDSLNKGIALLLENININEESKEKSEVISNGQTKDISSEAYKRYLQI |
| | QSEEKDIERLRTQIYFSLEDLEKALDLVLIDENMDRSDKSILSYVQSPDLN |
| | VNFERDLTDLYSRIMKLEENNEKLLANHSAIDLIKEFLDLIMLRYSRWQI |
| | LFCDSNYELDQTFYPIYDAVMEILSNIIRLYNLARNYLSRKPDRMKKKKI |
| | NFNNPTLADGWSESKIPDNSSMLFIKDGMYYLGIIKNRAAYSELLEAESL |
| | QSSEKKKSENSSYERMNYHFLPDAFRSIPKSSIAMKAVKEHFEINQKTAD |
| | LLLDTDKFSKPLRITKEIFDMQYVDLHKNKKKYQVDYLRDTGDKKGYR |
| | KALNTWLNFCKDFISKYKGRNLFDYSKIKDADHYETVNEFYNDVDKYS |
| | YHIFFTSVAETTVEKFISEGKLYLFQLYNKDFSPHSTGKPNLHTIYWRALF |
| | SEENLTSKNIKLNGQAEIFFRPKQIETPFTHKKGSILVNRFDVNGNPIPINV |
| | YQEIKGFKNNVIKWDDLNKTTQEGLENDQYLYFESEFEIIKDRRYTEDQL |
| | FFHVPISFNWDIGSNPKINDLATQYIVNSNDIHIIGIDRGENHLIYYSVIDLQ |
| | GAIVEQGSLNTITEYTENKFLNNKTNNLRKIPYKDILQQREDERADARIK |
| | WHAIDKIKDLKDGYLGQIVHFLAKLIIKYNAIVILEDLNYGFKRGRFKVE |
| | RQVYQKFEMALMKKLNVLVFKDYDIDEIGGPLKPWQLTRPIDSYERMG |
| | RQNGILFYVPAAYTSAVDPVTGFANLFYLNNVKNSEKFHFFSKFESIKYH |
| | SDQDMFSFAFDYNNFGTTTRINDLSKSKWQVFTNHERSVWNNKEKNYV |
| | TQNLTDLIKKLLRTYNIEFKNNQNVLDSILKIENNTDKENFARELFRLFRL |
| | TIQLRNTTVNENNTEITENELDYIISPVKDKNGNFFDSRDELKNLPDNGD |
| | ANGAYNIARKGLLYIEQLQESIKTGKLPTLSISTLDWFNYIMK* |
| SEQ ID NO: | MNKGGYVIMEKMTEKNRWENQFRITKTIKEEIIPTGYTKVNLQRVNMLK |
| 25 | REMERNEDLKKMKEICDEYYRNMIDVSLRLEQVRTLGWESLIHKYRML |
| | NKDEKEIKALEKEQEDLRKKISKGFGEKKAWTGEQFIKKILPQYLMDHY |
| | TGEELEEKLRIVKKFKGCTMFLSTFFKNRENIFTDKPIHTAVGHRITSENA |
| | MLFAANINTYEKMESNVTLEIERLQREFWRRGINISEIFTDAYYVNVLTQ |
| | KQIEAYNKICGDINQHMNEYCQKQKLKFSEFRMRELKKQILAVVGEHFE |
| | IPEKIESTKEVYRELNEYYESLKELHGQFEELKSVQLKYSQIYVQKKGYD |
| | RISRYIGGQWDLIQECMKKDCASGMKGTKKNHDAKIEEEVAKVKYQSIE |
| | HIQKLVCTYEEDRGHKVTDYVDEFIVSVCDLLGADHIITRDGERIELPLQ |
| | YEPGTDLLKNDTINQRRLSDIKTILDWHMDMLEWLKTFLVNDLVIKDEE |
| | FYMAIEELNERMQCVISVYNRIRNYVTQKGYEPEKIRICFDKGTILTGWT |
| | TGDNYQYSGFLLMRNDKYYLGIINTNEKSVRKILDGNEECKDENDYIRV |
| | GYHLINDASKQLPRIFVMPKAGKKSEILMKDEQCDYIWDGYCHNKHNES |
| | KEFMRELIDYYKRSIMNYDKWEGYCFKFSSTESYDNMQDFYKEVREQS |
| | YNISFSYINENVLEQLDKDGKIYLFQVYNKDFAAGSTGTPNLHTMYLQN |
| | LFSSQNLELKRLRLGGNAELFYRPGTEKDVTHRKGSILVDRTYVREEKD |
| | GIEVRDTVPEKEYLEIYRYLNGKQKGDLSESAKQWLDKVHYREAPCDII |
| | KDKRYAQEKYFLHFSVEINPNAEGQTALNDNVRRWLSEEEDIHVIGIDR |
| | GERNLIYVSLMDGKGRIKDQKSYNIVNSGNKEPVDYLAKLKVREKERDE |
| | ARRNWKAIGKIKDIKTGYLSYVVHEIVEMAVREKAIIVMEDLNYGFKRG |
| | RFKVERQVYQKFEEMLINKLNYVVDKQLSVDEPGGLLRGYQLAFIPKDK |
| | KSSMRQNGIVFYVPAGYTSKIDPTTGFVNIFKFPQFGKGDDDGNGKDYD |
| | KIRAFFGKFDEIRYECDEKVTADNTREVKERYRFDFDYSKFETHLVHMK |
| | KTKWTVYAEGERIKRKKVGNYWTSEVISDIALRMSNTLNIAGIEYKDGH |
| | NLVNEICALRGKQAGIILNELLEIVRLTVQLRNSTTEGDVDERDEIISPVLN |
| | EKYGCFYHSTEYKQQNGDVLPKDADANGAYCIGLKGIYEIRQIKNKWK |
| | EDMTKGEGKALNEGMRISHDQWFEFIQNMNKGE* |
| SEQ ID NO: | MNNPRGAFGGFTNLYSLSKTLRFELKPYLEIPEGEKGKLFGDDKEYYKN |
| 26 | CKTYTEYYLKKANKEYYDNEKVKNTDLQLVNFLHDERIEDAYQVLKPV |
| | FDTLHEEFITDSLESAEAKKIDFGNYYGLYEKQKSEQNKDEKKKIDKPLE |
| | TERGKLRKAFTPIYEAEGKNLKNKAGKEKKDKDILKESGFKVLIEAGILK |
| | YIKNNIDEFADKKLKNNEGKEITKKDIETALGAENIEGIFDGFFTYFSGFN |
| | QNRENYYSTEEKATAVASRIVDENLSKFCDNILLYRKNENDYLKIFNFLK |
| | NKGKDLKLKNSKFGKENEPEFIPAYDMKNDEKSFSVADFVNCLSQGEIE |
| | KYNAKIANANYLINLYNQNKDGNSSKLSMFKILYKQIGCGEKKDFIKTIK |
| | DNAELKQILEKACEAGKKYFIRGKSEDGGVSNIFDFTDYIQSHENYKGVY |
| | WSDKAINTISGKYFANWDTLKNKLGDAKVENKNTGEDKADVKYKVPQ |
| | AVMLSELFAVLDDNAGEDWREKGIFFKASLFEGDQNKSEIIKNANRPSQ |
| | ALLKMICDDMESLAKNFIDSGDKILKISDRDYQKDENKQKIKNWLDNAL |
| | WINQILKYFKVKANKIKGDSIDARIDSGLDMLVFSSDNPAEDYDMIRNYL |
| | TQKPQDEINKLKLNFENSSLAGGWDENKEKDNSCIILKDEQDKQYLAVM |
| | KYENTKVFEQKNSQLYIADNAAWKKMIYKLVPGASKTLPKVFFSKKWT |
| | ANRPTPSDIVEIYQKGSFKKENVDFNDKKEKDESRKEKNREKIIAELQKT |
| | CWMDIRYNIDGKIESAKYVNKEKLAKLIDFYKENLKKYPSEEESWDRLF |
| | AFGFSDTKSYKSIDQFYIEVDKQGYKLEFVTINKARLDEYVRDGKIYLFEI |
| | RSRDNNLVNGEEKTSAKNLQTIYWNAAFGGDDNKPKLNGEAEIFYRPAI |
| | AENKLNKKKDKNGKEIIDGYRFSKEKFIFHCPITLNFCLKETKINDKLNA |
| | ALAKPENGQGVYFLGIDRGEKHLAYYSLVNQKGEILEQGTLNLPFLDKN |
| | GKSRSIKVEKKSFEKDSNGGIIKDKDGNDKIKIEFVECWNYNDLLDARAG |
| | DRDYARKNWTTIGTIKELKDGYISQVVRKIVDLSIYKNTETKEFREMPAF |
| | IVLEDLNIGFKRGRQKIEKQVYQKLELALAKKLNFLVDKKADIGEIGSVT |
| | KAIQLTPPVNNFGDMENRKQFGNMLYIRADYTSQTDPATGWRKSIYLKS |
| | GSESNVKEQIEKSFFDIRYESGDYCFEYRDRHGKMWQLYSSKNGVSLDR |
| | FHGERNNSKNVWESEKQPLNEMLDILFDEKRFDKSKSLYEQMFKGGVA |
| | LTRLPKEINKKDKPAWESLRFVIILIQQIRNTGKNGDDRNGDFIQSPVRDE |
| | KTGEHFDSRIYLDKEQKGEKADLPTSGDANGAYNIARKGIVVAEHIKRG |
| | FDKLYISDEEWDTWLAGDEIWDKWLKENRESLTKTRK* |
| SEQ ID NO: | MNYKTGLEDFIGKESLSKTLRNALIPTESTKIHMEEMGVIRDDELRAEKQ |
| 27 | QELKEIMDDYYRTFIEEKLGQIQGIQWNSLFQKMEETMEDISVRKDLDKI |
| | QNEKRKEICCYFTSDKRFKDLFNAKLITDILPNFIKDNKEYTEEEKAEKEQ |
| | TRVLFQRFATAFTNYFNQRRNNFSEDNISTAISFRIVNENSEIHLQNMRAF |
| | QRIEQQYPEEVCGMEEEYKDMLQEWQMKHIYSVDFYDRELTQPGIEYY |
| | NGICGKINEHMNQFCQKNRINKNDFRMKKLHKQILCKKSSYYEIPFRFES |
| | DQEVYDALNEFIKTMKKKEIIRRCVHLGQECDDYDLGKIYISSNKYEQIS |
| | NALYGSWDTIRKCIKEEYMDALPGKGEKKEEKAEAAAKKEEYRSIADID |
| | KIISLYGSEMDRTISAKKCITEICDMAGQISIDPLVCNSDIKLLQNKEKTTEI |
| | KTILDSFLHVYQWGQTFIVSDIIEKDSYFYSELEDVLEDFEGITTLYNHVR |
| | SYVTQKPYSTVKFKLHFGSPTLANGWSQSKEYDNNAILLMRDQKFYLGI |
| | FNVRNKPDKQIIKGHEKEEKGDYKKMIYNLLPGPSKMLPKVFITSRSGQE |
| | TYKPSKHILDGYNEKRHIKSSPKFDLGYCWDLIDYYKECIHKHPDWKNY |
| | DFHFSDTKDYEDISGFYREVEMQGYQIKWTYISADEIQKLDEKGQIFLFQI |
| | YNKDFSVHSTGKDNLHTMYLKNLFSEENLKDIVLKLNGEAELFFRKASI |
| | KTPIVHKKGSVLVNRSYTQTVGNKEIRVSIPEEYYTEIYNYLNHIGKGKLS |
| | SEAQRYLDEGKIKSFTATKDIVKNYRYCCDHYFLHLPITINFKAKSDVAV |
| | NERTLAYIAKKEDIHIIGIDRGERNLLYISVVDVHGNIREQRSFNIVNGYD |
| | YQQKLKDREKSRDAARKNWEEIEKIKELKEGYLSMVIHYIAQLVVKYN |
| | AVVAMEDLNYGFKTGRFKVERQVYQKFETMLIEKLHYLVFKDREVCEE |
| | GGVLRGYQLTYIPESLKKVGKQCGFIFYVPAGYTSKIDPTTGFVNLFSFK |
| | NLTNRESRQDFVGKFDEIRYDRDKKMFEFSFDYNNYIKKGTILASTKWK |
| | VYTNGTRLKKIVVNGKYTSQSMEVELTDAMEKMLQRAGIEYHDGKDL |
| | KGQIVEKGIEAEIIDIFRLTVQMRNSRSESEDREYDRLISPVLNDKGEFFDT |
| | ATADKTLPQDADANGAYCIALKGLYEVKQIKENWKENEQFPRNKLVQD |
| | NKTWFDFMQKKRYL* |
| SEQ ID NO: | MRISKTLSLRIVRPFYTPEVEAGIKAEKDKREAQGQTRSLDAKFFNELKK |
| 28 | KHSEIILSSEFYSLLSEVQRQLTSIYNHAMSNLYHKIIVEGEKTSTSKALSN |
| | IGYDECKAIFPSYMALGLRQKIQSNFRRRDLKNFRMAVPTAKSDKFPIPIY |
| | RQVDGSKGGFKISENDGKDFIVELPLVDYVAEEVKTAKGRFTKINISKPP |
| | KIKNIPVILSTLRRRQSGQWFSDDGTNAEIRRVISGEYKVSWIEIVRRTRF |
| | GKHDDWFVNMVIKYDKPEEGLDSKVVGGIDVGVSSPLVCALNNSLDRY |
| | FVKSSDIIAFNKRAMARRRTLLRQNKYKRSGHGSKNKLEPITVLTEKNER |
| | FKKSIMQRWAKEVAEFFRGKGASVVRMEELSGLKEKDNFFSSYLRMYW |
| | NYGQLQQIIENKLKEYGIKVNYVSPKDTSKKCHSCTHINEFFTFEYRQKN |
| | NFPLFKCEKCGVECSADYNAAKNMAIA |
| SEQ ID NO: | MRTMVTFEDFTKQYQVSKTLRFELIPQGKTLENMKRDGIISVDRQRNED |
| 29 | YQKAKGILDKLYKYILDFTMETVVIDWEALATATEEFRKSKDKKTYEKV |
| | QSKIRTALLEHVKKQKVGTEDLFKGMFSSKIITGEVLAAFPEIRLSDEENL |
| | ILEKFKDFTTYFTGFFENRKNVFTDEALSTSFTYRLVNDNFIKFFDNCIVF |
| | KNVVNISPHMAKSLETCASDLGIFPGVSLEEVFSISFYNRLLTQTGIDQFN |
| | QLLGGISGKEGEHKQQGLNEIINLAMQQNLEVKEVLKNKAHRFTPLFKQI |
| | LSDRSTMSFIPDAFADDDEVLSAVDAYRKYLSEKNIGDRAFQLISDMEAY |
| | SPELMRIGGKYVSVLSQLLFYSWSEIRDGVKAYKESLITGKKTKKELENI |
| | DKEIKYGVTLQEIKEALPKKDIYEEVKKYAMSVVKDYHAGLAEPLPEKI |
| | ETDDERASIKHIMDSMLGLYRFLEYFSHDSIEDTDPVFGECLDTILDDMN |
| | ETVPLYNKVRNFSTRKVYSTEKFKLNFNNSSLANGWDKNKEQANGAILL |
| | RKEGEYFLGIFNSKNKPKLVSDGGAGIGYEKMIYKQFPDFKKMLPKCTIS |
| | LKDTKAHFQKSDEDFTLQTDKFEKSIVITKQIYDLGTQTVNGKKKFQVD |
| | YPRLTGDMEGYRAALKEWIDFGKEFIQAYTSTAIYDTSLFRDSSDYPDLP |
| | SFYKDVDNICYKLTFEWIPDAVIDDCIDDGSLYLFKLHNKDFSSGSIGKPN |
| | LHTLYWKALFEEENLSDVVVKLNGQAELFYRPKSLTRPVVHEEGEVIIN |
| | KTTSTGLPVPDDVYVELSKFVRNGKKGNLTDKAKNWLDKVTVRKMPH |
| | AITKDRRFTVDKFFFHVPITLNYKADSSPYRFNDFVRQYIKDCSDVKIIGI |
| | DRGERNLIYAVVIDGKGNIIEQRSFNTVGTYNYQEKLEQKEKERQTARQ |
| | DWATVTKIKDLKKGYLSAVVHELSKMIVKYKAIVALENLNVGFKRMRG |
| | GIAERSVYQQFEKALIDKLNYLVFKDEEQSGYGGVLNAYQLTDKFESFS |
| | KMGQQTGFLFYVPAAYTSKIDPLTGFINPFSWKHVKNREDRRNFLNLFS |
| | KLYYDVNTHDFVLAYHHSNKDSKYTIKGNWEIADWDILIQENKEVFGK |
| | TGTPYCVGKRIVYMDDSTTGHNRMCAYYPHTELKKLLSEYGIEYTSGQD |
| | LLKIIQEFDDDKLVKGLFYIIKAALQMRNSNSETGEDYISSPIEGRPGICFD |
| | SRAEADTLPYDADANGAFHIAMKGLLLTERIRNDDKLAISNEEWLNYIQ |
| | EMRG* |
| SEQ ID NO: | MSKLEKFTNCYSLSKTLRFKAIPVGKTQENIDNKRLLVEDEKRAEDYKG |
| 30 | VKKLLDRYYLSFINDVLHSIKLKNLNNYISLFRKKTRTEKENKELENLEIN |
| | LRKEIAKAFKGNEGYKSLFKKDIIETILPEFLDDKDEIALVNSFNGFTTAFT |
| | GFFDNRENMFSEEAKSTSIAFRCINENLTRYISNMDIFEKVDAIFDKHEVQ |
| | EIKEKILNSDYDVEDFFEGEFFNFVLTQEGIDVYNAIIGGFVTESGEKIKGL |
| | NEYINLYNQKTKQKLPKFKPLYKQVLSDRESLSFYGEGYTSDEEVLEVFR |
| | NTLNKNSEIFSSIKKLEKLFKNFDEYSSAGIFVKNGPAISTISKDIFGEWNV |
| | IRDKWNAEYDDIHLKKKAVVTEKYEDDRRKSFKKIGSFSLEQLQEYADA |
| | DLSVVEKLKEIIIQKVDEIYKVYGSSEKLFDADFVLEKSLKKNDAVVAIM |
| | KDLLDSVKSFENYIKAFFGEGKETNRDESFYGDFVLAYDILLKVDHIYDA |
| | IRNYVTQKPYSKDKFKLYFQNPQFMGGWDKDKETDYRATILRYGSKYY |
| | LAIMDKKYAKCLQKIDKDDVNGNYEKINYKLLPGPNKMLPKVFFSKKW |
| | MAYYNPSEDIQKIYKNGTFKKGDMFNLNDCHKLIDFFKDSISRYPKWSN |
| | AYDFNFSETEKYKDIAGFYREVEEQGYKVSFESASKKEVDKLVEEGKLY |
| | MFQIYNKDFSDKSHGTPNLHTMYFKLLFDENNHGQIRLSGGAELFMRRA |
| | SLKKEELVVHPANSPIANKNPDNPKKTTTLSYDVYKDKRFSEDQYELHIP |
| | IAINKCPKNIFKINTEVRVLLKHDDNPYVIGIDRGERNLLYIVVVDGKGNI |
| | VEQYSLNEIINNFNGIRIKTDYHSLLDKKEKERFEARQNWTSIENIKELKA |
| | GYISQVVHKICELVEKYDAVIALEDLNSGFKNSRVKVEKQVYQKFEKML |
| | IDKLNYMVDKKSNPCATGGALKGYQITNKFESFKSMSTQNGFIFYIPAW |
| | LTSKIDPSTGFVNLLKTKYTSIADSKKFISSFDRIMYVPEEDLFEFALDYK |
| | NFSRTDADYIKKWKLYSYGNRIRIFRNPKKNNVFDWEEVCLTSAYKELF |
| | NKYGINYQQGDIRALLCEQSDKAFYSSFMALMSLMLQMRNSITGRTDV |
| | DFLISPVKNSDGIFYDSRNYEAQENAILPKNADANGAYNIARKVLWAIGQ |
| | FKKAEDEKLDKVKIAISNKEWLEYAQTSVKH |
| SEQ ID NO: | MTNFDNFTKKYVNSKTIRLEAIPVGKTLKNIEKMGFIAADRQRDEDYQK |
| 31 | AKSVIDHIYKAFMDDCLKDLFLDWDPLYEAVVACWRERSPEGRQALQI |
| | MQADYRKKIADRFRNHELYGSLFTKKIFDGSVAQRLPDLEQSAEEKSLLS |
| | NFNKFTSYFRDFFDKRKRLFSDDEKHSAIAYRLINENFLKFVANCEAFRR |
| | MTERVPELREKLQNTGSLQVYNGLALDEVFSADFYNQLIVQKQIDLYNQ |
| | LIGGIAGEPGTPNIQGLNATINLALQGDSSLHEKLAGIPHRFNPLYKQILSD |
| | VSTLSFVPSAFQSDGEMLAAVRGFKVQLESGRVLQNVRRLENGLETEAD |
| | LSRVYVNNSKLAAFSSMFFGRWNLCSDALFAWKKGKQKKITNKKLTEI |
| | KKWLKNSDIAIAEIQEAFGEDFPRGKINEKIQAQADALHSQLALPIPENLK |
| | ALCAKDGLKSMLDTVLGLYRMLQWFIVGDDNEKDSDFYFGLGKILGSL |
| | DPVLVLYNRVRNYITKKPYSLTKFRLNFDNSQLLNGWDENNLDTNCASI |
| | FIKDGKYYLGISNKNNRPQFDTVATSGKSGYQRMVYKQFANWGRDLPH |
| | STTQMKKVKKHFSASDADYVLDGDKFIRPLIITKEIFDLNNVKFNGKKKL |
| | QVDYLRNTGDREGYTHALHTWINFAKDFCACYKSTSIYDISCLRPTDQY |
| | DNLMDFYADLGNLSHRIVWQTIPEEAIDNYVEQGQLFLFQLYNKDFAPG |
| | ADGKPNLHTLYWKAVFNPENLEDVVVKLNGKAELFYRPRSNMDVVRH |
| | KVGEKLVNRKLKNGLTLPSRLHEEIYRYVNGTLNKDLSADARSVLPLAV |
| | VRDVQHEIIKDRRFTADKFFFHASLTFNFKSSDKPVGFNEDVREYLREHP |
| | DTYVVGVDRGERNLIYIVVIDPQGNIVEQRSFNMINGIDYWSLLDQKEKE |
| | RVEAKQAWETVGKIKDLKCGYLSFLIHEITKIIIKYHAVVILENLSLGFKR |
| | VRTGIAEKAVYQQFERMLVTKLGYVVFKDRAGKAPGGVLNAYQLTDN |
| | TRTAENTGIQNGFLFYVPAAFTSRVDPATGFFDFYDWGKIKTATDKKNFI |
| | AGFNSVRYERSTGDFIVHVGAKNLAVRRVAEDVRTEWDIVIEANVRKM |
| | GIDGNSYISGKRIRYRSGEQGHGQYENHLPCQELIRALQQYGIQYETGKD |
| | ILPAILQQDDAKLTDTVFDVFRLALQMRNTSAETGEDYFNSVVRDRSGR |
| | CFDTRRAEAAMPKEADANDAYHIALKGLFVLEKLRKGESIGIKNTEWLR |
| | YVQQRHS* |
| SEQ ID NO: | MTPIFCNFVVYQIMLFNNNININVKTMNKKHLSDFTNLFPVSKTLRFRLE |
| 32 | PQGKTMENIVKAQTIETDEERSHDYEKTKEYIDDYHRQFIDDTLDKFAFK |
| | VESTGNNDSLQDYLDAYLSANDNRTKQTEEIQTNLRKAIVSAFKMQPQF |
| | NLLFKKEMVKHLLPQFVDTDDKKRIVAKFNDFTTYFTGFFTNRENMYSD |
| | EAKSTSIAYRIVNQNLIKFVENMLTFKSHILPILPQEQLATLYDDFKEYLN |
| | VASIAEMFELDHFSIVLTQRQIEVYNSVIGGRKDENNKQIKPGLNQYINQ |
| | HNQAVKDKSARLPLLKPLFNQILSEKAGVSFLPKQFKSASEVVKSLNEAY |
| | AELSPVLAAIQDVVTNITDYDCNGIFIKNDLGLTDIAQRFYGNYDAVKRG |
| | LRNQYELETPMHNGQKAEKYEEQVAKHLKSIESVSLAQINQVVTDGGDI |
| | CDYFKAFGATDDGDIQRENLLASINNAHTAISPVLNKENANDNELRKNT |
| | MLIKDLLDAIKRLQWFAKPLLGAGDETNKDQVFYGKFEPLYNQLDETIS |
| | PLYDKVRSYLTKKPYSLDKFKINFEKSNLLGGWDPGADRKYQYNAVILR |
| | KDNDFYLGIMRDEATSKRKCIQVLDCNDEGLDENFEKVEYKQIKPSQNM |
| | PRCAFAKKECEENADIMELKRKKNAKSYNTNKDDKNALIRHYQRYLDR |
| | TYPEFGFVYKDADEYDTVKAFTDSMDSQDYKLSFLQVSETGLNKLVDE |
| | GDLYLFKITNKDFSSYAKGRPNLHTIYWRMLFDPKNLANVVYKLEGKA |
| | EVFFRRKSLASTTTHKAKQAIKNKSRYNEAVKPQSTFDYDIIKDRRFTAD |
| | KFEFHVPIKMNFKAAGWNSTRLTNEVREFIKSQGVRHIIGIDRGERHLLY |
| | LTMIDMDGNIVKQCSLNAPAQDNARASEVDYHQLLDSKEADRLAARRN |
| | WGTIENIKELKQGYLSQVVHLLATMMVDNDAILVLENLNAGFMRGRQK |
| | VEKSVYQKFEKMLIDKLNYIVDKGQSPDKPTGALHAVQLTGLYSDFNKS |
| | NMKRANVRQCGFVFYIPAWNTSKIDPVTGFVNLFDTHLSSMGEIKAFFS |
| | KFDSIRYNQDKGWFEFKFDYSRFTTRAEGCRTQWTVCTYGERIWTHRSK |
| | NQNNQFVNDTVNVTQQMLQLLQDCGIDPNGNLKEAIANIDSKKSLETLL |
| | HLFKLTVQMRNSVTGSEVDYMISPVADERGHFFDSRESDEHLPANADAN |
| | GAFNIARKGLMVVRQIMATDDVSKIKFAVSNKDWLRFAQHIDD* |
| SEQ ID NO: | VKISKTLSLRIIRPYYTPEVESAIKAEKDKREAQGQTRNLDAKFFNELKKK |
| 33 | HPQIILSGEFYSLLFEMQRQLTSIYNRAMSSLYHKIIVEGEKTSTSKALSDI |
| | GYDECKSVFPSYIALGLRQKIQSNFRRKELKGFRMAVPTAKSDKFPIPIYK |
| | QVDDGKGGFKISENKEGDFIVELPLVEYTAEDVKTAKGKFTKINISKPPKI |
| | KNIPVILSTLRRKQSGQWFSDEGTNAEIRRVISGEYKVSWIEVVRRTRFG |
| | KHDDWFLNIVIKYDKTEDGLDPEVVGGIDVGVSTPLVCAVNNSLDRYFV |
| | KSSDIIAFKKRAMARRRTLLRQNRFKRSGHGSKSKLEPITILTEKNERFKK |
| | SIMQRWAKEVAEFFKGERASVVQMEELSGLKEKDNFFGSYLRMYWNY |
| | GQLQQIIENKLKEYGIKVNYVSPKDTSKKCHSCGYINEFFTFEFRQKNNFP |
| | LFKCKKCGVECNADYNAAKNIAIA |
| SEQ ID NO: | MLKSYDYFTKLYSLQKTLRFELKPIGKTLEHIKNSGIIESDETLEEQYAIV |
| 34 | KNIIDKLHRKHIDEALSLVDFTKHLDTLKTFQELYLKRGKTDKEKEELEK |
| | LSADLRKLIVSYLKGNVKEKTQHNLNPIKERFEILFGKELFTNEEFFLLAE |
| | NEKEKKAIQAFKGFTTYFKGFQENRKNMYSEEGNSTSIAYRIINENLPLFI |
| | ENIARFQKVMSTIEKTTIKKLEQNLKTELKKHNLPGIFTIEYFNNVLTQEG |
| | ISRYNTIIGGKTTHEGVKIQGLNEIINLYNQQSKDVKLPILKPLHKQILSEE |
| | YSTSFKIKAFENDNEVLKAIDTFWNEHIEKSIHPVTGNKFNILSKIENLCD |
| | QLQKYKDKDLEKLFIERKNLSTVSHQVYGQWNIIRDALRMHLEMNNKN |
| | IKEKDIDKYLDNDAFSWKEIKDSIKIYKEHVEDAKELNENGIIKYFSAMSI |
| | NEEDDEKEYSISLIKNINEKYNNVKSILQEDRTGKSDLHQDKEKVGIIKEF |
| | LDSLKQLQWFLRLLYVTVPLDEKDYEFYNELEVYYEALLPLNSLYNKVR |
| | NYMTRKPYSVEKFKLNFNSPTLLDGWDKNKETANLSIILRKNGKYYLGI |
| | MNKENNTIFEYYPGTKSNDYYEKMIYKLLPGPNKMLPKVFFSKKGLEYY |
| | NPPKEILNIYEKGEFKKDKSGNFKKESLHTLIDFYKEAIAKNEDWEVFNF |
| | KFKNTKEYEDISQFYRDVEEQGYLITFEKVDANYVDKLVKEGKLYLFQI |
| | YNKDFSENKKSKGNPNLHTIYWKGLYDSENLKNVVYKLNGEAEVFYRK |
| | KSIDYPEEIYNHGHHKEELLGKFNYPIIKDRRYTQDKFLFHVPITMNFISK |
| | EEKRVNQLACEYLSATKEDVHIIGIDRGERHLLYLSLIDKEGNIKKQLSLN |
| | TIKNENYDKEIDYRVKLDEKEKKRDEARKNWDVIENIKELKEGYMSQVI |
| | HIIAKMMVEEKAILIMEDLNIGFKRGRFKVEKQVYQKFEKMLIDKLNYL |
| | VFKNKNPLEPGGSLNAYQLTSKFDSFKKLGKQSGFIFYVPSAYTSKIDPTT |
| | GFYNFIQVDVPNLEKGKEFFSKFEKIIYNTKEDYFEFHCKYGKFVSEPKN |
| | KDNDRKTKESLTYYNAIKDTVWVVCSTNHERYKIVRNKAGYYESHPVD |
| | VTKNLKDIFSQANINYNEGKDIKPIIIESNNAKLLKSIAEQLKLILAMRYN |
| | NGKHGDDEKDYILSPVKNKQGKFFCTLDGNQTLPINADANGAYNIALKG |
| | LLLIEKIKKQQGKIKDLYISNLEWFMFMMSR |
| SEQ ID NO: | MIKNPSNRHSLPKVIISEVDHEKILEFKIKYEKLARLDRFEVKAMHYEGK |
| 88 | EIVFDEVLVNGGLIEVEYQDDNKTLFVKVGEKSYSIRGKKVGGKQRLLE |
| | DRVSKTKVQLELSDGVVDNKGNLRKSRTERELIVADNIKLYSQIVGREV |
| | TTTKEIYLVKRFLAYRSDLLFYYSFVDNFFKVAGNEKELWKINFDDATS |
| | AQFMGYIPFMVNDNLKNDNAYLKDYVRNDVQIKDDLKKVQTIFSALRH |
| | TLLHFNYEFFEKLFNGEDVGFDFDIGFLNLLIENIDKLNIDAKKEFIDNEKI |
| | RLFGENLSLAKVYRLYSDICVNRVGFNKFINSMLIKDGVENQVLKAEFN |
| | RKFGGNAYTIDIHSNQEYKRIYNEHKKLVIKVSTLKDGQAIRRGNKKISE |
| | LKEQMKSMTKKNSLARLECKMRLAFGFLYGEYNNYKAFKNNFDTNIKN |
| | SQFDVNDVEKSKAYFLSTYERRKPRTREKLEKVAKDIESLELKTVIANDT |
| | LLKFILLMFVFMPQELKGDFLGFVKKYYHDVHSIDDDTKEQEEDVVEA |
| | MSTSLKLKILGRNIRSLTLFKYALSSQVNYNSTDNIFYVEGNRYGKIYKK |
| | LGISHNQEEFDKTLVVPLLRYYSSLFKLMNDFEIYSLAKANPTAVSLQEL |
| | VDDETSPYKQGNYFNFNKMLRDIYGLTSDEIKSGQVVFMRNKIAHFDTE |
| | VLLSKPLLGQTKMNLQRKDIVSFIEARGDIKELLGYDAINDFRMKVIHLR |
| | TKMRVYSDKLQTMMDLLRNAKTPNDFYNVYKVKGVESINKHLLEVLA |
| | QTAEERTVEKQIRDGNEKYDL |
| SEQ ID NO: | LNSIEKIKKPSNRNSIPSIIISDYDENKIKEIKVKYLKLARLDKITIQDMEIRD |
| 89 | NIVEFKKILLNGIEHTIKDNQKIEFDNYEITAYVRASKQRRDGKITQAKYV |
| | VTITDKYLRDNEKEKRFKSTERELPNDTLLMRYKQISGFDTLTSKDIYKIK |
| | RYIDFKNEMLFYFQFIEEFFSPLLPKGTNFYSLNIEQNKDKVVKYIVYRLN |
| | DDFKNQSLNQFIKKTDTIKYDFLKIQKILSDFRHALAHFDFDFIQKFFDDE |
| | LDKNRFDISTISLIKTMLQEKEEKYYQEKNNYIEDSDTLTLFDEKESNFSK |
| | IHNFYIKISQKKPAFNKLINSFLSKDGVPNEELKSYLATKKIDFFEDIHSNK |
| | EYKKIYIKHKNLVVEKQKEESQEKPNGQKLKNYNDELQKLKDEMNKIT |
| | KQNSLNRLEVKLRLAFGFIANEYNYNFKNFNDKFTLDVKKEQKIKVFKN |
| | SSNEKLKEYFESTFIEKRFFHFCVKFFNKKTKKEETKQKNIFNLIENETLEE |
| | LVKESPLLQIITLLYLFIPKELQGEFVGFILKIYHHTKNITNDTKEDEKSIED |
| | TQNSFSLKLKILAKNLRGLQLFNYSLSHNTLYNTKEHFFYEKGNRWQSV |
| | YKSLEISHNQDEFDIHLVIPVIKYYINLNKLIGDFEIYALLTYADKNSITEK |
| | LSDITKRDDLKFRGYYNFSTLLFKTFMINTNYEQNQKSTQYIKQTRNDIA |
| | HQNIENMLKAFENNEIFAQREEIVNYLQKEHKMQEILHYNPINDFTMKT |
| | VQYLKSLNIHSQKESKIADIHKKESLVPNDYYLIYKLK VIELLKQKVIEAI |
| | GETKDEEKIKNAIAKEEQIKKGYNK |
| SEQ ID NO: | MLKHKRKNKNSLARVVLSNYDSNNIYEIKIKYEKLAKLDKINIIEMDYD |
| 90 | ADNNVMFKKVLFNNKEIDLSHKDKTKINIELDNKKYNISAKKQIGKTHL |
| | VVRNKQTSKISRIKKIQDTYYRGKDVFILDNNIEILDKKQTKDKFIVTLND |
| | ITNNKTTSTEAELIDDTKDIFKKISAKKDLKSSDIYKIKRFISIRSNFSFYYT |
| | FVDNYFKIFHAKKDKNKEELYKIKFKDEINIKPYLENILDNMKNKNGILY |
| | NYANDRKKVLNDLRNIQYVFKEFRHKLAHFDYNFLDNFFSNSVEEKYK |
| | QKVNEIKLLDILLDNIDSLNVVPKQNYIEDETISVFDAKDIKLKRLYTYYI |
| | KLTINYPGFKKLINSFFIQDGIENQELKEYINNKEKDTQVLKELDNKAYY |
| | MDISQYRKYKNIYNKHKELVSEKELSSDGKKINSLNQKINKLKIDMKNIT |
| | KPNALNRLIYRLRVAFGFIYKEYATINNFNKSFLQDTKTKRFENISQQDIK |
| | SYLDISYQDKGKFFVKSKKTFKNKTTVKYTFEDLDLTLNEIITQDDIFVK |
| | VIFLFSIFMPKELNGDFFGFINMYYHKMKNISYDTKDIDMLDTISQNMKL |
| | KILEQNIKKTYVFKYYLDLDSSIYSKLVQNIKITEDIDSKKYLYAKIFKYY |
| | QHLYKLISDVEIYLLYKYNSKENLSITIDKDELKHRGYYNFQSLLIKNNIN |
| | KDDAYWSIVNMRNNLSHQNIDELVGHFCKGCLRKSTTDIAELWLRKDIL |
| | TITNEIINKIESFKDIKITLGYDCVNDFTQKVKQYKQKLKASNERLAKKIE |
| | EKQNQVVDEKNKEELEKNILNMKNIQKINRYILDIL |
| SEQ ID NO: | MLKHKRKNKNSLARVVLSNYDSNNIYEIKIKYEKLAKLDKINIIEMDYD |
| 91 | ADNNVMFKKVLFNNKEIDLSHKDKTKINIELDNKKYNISAKKQIGKTHL |
| | VVRDKQTSKISRIKKIQDTYYRGKDVFILDNNIEILDKKQTKDKFIVTLND |
| | ITNDKTTSTEAELIDDTKDIFKKISAKKDLKSSDIYKIKRFISIRSNFSFYYT |
| | FVDNYFKIFHAKKDKNKEELYKIKFKDEINIKPYLENILDNMKNKNGILY |
| | DYADDREKVLNDLKNIQYVFTEFRHKLAHFDYNFLDNFFSNSVTDQYK |
| | QKVNEIKLLDILLDNIDSLNVVPKQNYIEDETISVFDAKDIKLKRLYTYYI |
| | KLTINYPGFKKLINSFFIQDGIENQELKEYINNKEKDTQVLKELDNKAYY |
| | MDISQYRKYKNIYNKHKELVSEKELSSDGQKINSLNQKINKLKIEMKNIT |
| | KPNALNRLIYRLRVAFGFIYKEYATINNFNKSFLQDTKIKRFENISQQDIK |
| | NYLDISYQDKGKFFVKSKKTFKNKTTIKYTFEDLDLTLNEIITQDDIFVKV |
| | IFLFSIFMPKELNGDFFGFINMYYHKMKNISYDTKDIDMLDTISQNMKLKI |
| | LEQNIKKTYVFKYYLDLDSSIYSKLVQNIKITEDIDSKKYLYAKIFKYYQH |
| | LYKLISDVEIYLLYKYNSKENLSITIDKDELKHRGYYNFQSLLIKNNINKD |
| | DAYWSIVNMRNNLSHQNIDELVGHFCKGCLRKSTTDIAELWLRKDILTIT |
| | NEIINKIESFKDIKITLGYDCVNDFTQKVKQYKQKLKASNERLAKKIEEK |
| | QNQVVDEKNKEELEKKILNMKNIQKINRYILDIL |
| SEQ ID NO: | MSQLKNPSNKNSLPRIIISDFNEIKINEIKIKYHKLDRLDKIIVKEMEIINNKI |
| 92 | FFKKILFNNQIKDINSENIELENYILAGEVKPSNTKIILNRDGKEKSFIVYD |
| | GFTFKYKPNDKRISETKTNAKYILTIKDKTRHRESSTQRDILKSSIIETYKQ |
| | ISGFENITSKDIYTIKRYIDFKNEMMFYYTFIDDFFFPITGKNKQDKKNNF |
| | YNYKIKENAKKFISLINYRINDDFKNKNGILYDYLSNKEEIIINDFIHIQTIL |
| | KDVRHAIAHFNFDFIQKLFDNEQAFNSKFDGIEILNILFNQKQEKYFEAQT |
| | NYIEEETIKILDEKELSFKKLHSFYSQICQKKPAFNKLINSFIIQDGIENKEL |
| | KDYISQKYNSKFDYYLDIHTCKIYKDIYNQHKKFVADKQFLENQKTDGQ |
| | KIKKLNDQINQLKTKMNNLTKKNSLKRLEIKFRLAFGFIFTEYQTFKNFN |
| | ERFIEDIKANKYSTKIELLDYGKIKEYISITHEEKRFFNYKTFNKKTNKNIN |
| | KTIFQSLEKETFENLVKNDNLIKMMFLFQLLLPRELKGEFLGFILKIYHDL |
| | KNIDNDTKPDEKSLSELNISTALKLKILVKNIRQINLFNYTISNNTKYEEKE |
| | KRFYEEGNQWKDIYKKLYISHDFDIFDIHLIIPIIKYNINLYKLIGDFEVYLL |
| | LKYLERNTNYKTLDKLIEAEELKYKGYYNFTTLLSKAINIALNDKEYHNI |
| | THLRNNTSHQDIQNIISSFKNNKLLEQRENIIELISKESLKKKLHFDPINDFT |
| | MKTLQLLKSLEVHSDKSEKIENLLKKEPLLPNDVYLLYKLKGIEFIKKELI |
| | SNIGITKYEEKIQEKIAKGVEK |
| SEQ ID NO: | MVKNPANRHALPKVIISEVDNNNILEFKIKYEKLARLDKVEVKSMHFDN |
| 93 | NKQVVFDEVVINGGLIEPTYEDKHKKLVVTAGEKSYSIVGQKVGGKPRL |
| | LEDRVSKTKVQLELTNYVEDKEGKKRVSKTERELIVADNIELYSQIVGRE |
| | VKTTKEIYLIKRFLEYRSDLLFYYGFVDNFFKVAGNGKELWKIDFTNSDS |
| | LHLIEYFKFSINDNLKNDENYLKNYVSDNTKIENDLVKCQNNFNSLRHA |
| | LMHFDYDFFEKLFNGEDVGFDFDIEFLNIMIDKVDKLNIDTKKEFIDDEE |
| | VTLFGEALSLKKLYGLFSHIAINRVAFNKLINSFIIEDGIENKELKDFFNNK |
| | KESQAYEIDIHSNAEYKALYVQHKKLVMATSAMTDGDEIAKKNQEISDL |
| | KEKMKVITKENSLARLEHKLRLAFGFIYTEYKDYKTFKKHFDQDIKGAK |
| | YKGLNVEKLKEYYETTLKNSKPKTDEKLEDVAKKIDKLSLKELIDDDTL |
| | LKFVLLLFIFMPQELKGDFLGFIKKYYHDKKHIDQDTKDKDTEIEELSTG |
| | LKLKVLDKNIRSLSILKHSFSFQVKYNRKDKNFYEDGNLHGKFYKKLSIS |
| | HNQEEFNKSVYAPLFRYYSALYKLINDFEIYALAQHVENHETLADQVNK |
| | SQFIQKSYFNFRKLLDNTDSISQSSSYNTLIVMRNDISHLSYEPLFNYPLDE |
| | RKSYKKKTQKGVKTFHVELLYISRAKIIELISLQTDMKKLLGYDAVNDEN |
| | MKVVHLRKRLSVYANKEESIRKMQADAKTPNDFYNIYKVKGVESINQH |
| | LLKVIGVTEAEKSIEKQINEGNKKHNT |
| SEQ ID NO: | MTKKPSNRNSLPKVIINKVDESSILEFKIKYEKLARLDRFEVRSMRYDGD |
| 94 | GRIIFDEVVANAGLLDVDYEDDNRTIVVKIENKAYNIYGKKVGGEKRLN |
| | GKISKAKVQLILTDSIRKNANDTHRHSLTERELINKNEVDLYSKIAEREIS |
| | TTKDIYLVKRFLAYRSDLLLYYAFINHYVRVNGNKKEFWKTEIDDKIIDY |
| | FIYTINDTLKNKEGYLEKYIVDRDQIKKDLEKIKQIFSHLRHKLMHYDFRF |
| | FTDLFDGKDVDIKVDNSIQKISELLDIEFLNIVIDKLEKLNIDAKKEFIDDE |
| | KITLFGQEIELKKLYSLYAHTSINRVAFNKLINSFLIKDGVENKELKEYFN |
| | AHNQGKESYYIDIHQNQEYKKLYIEHKNLVAKLSATTDGKEIAKINRELA |
| | DKKEQMKQITKANSLKRLEYKLRLAFGFIYTEYKDYERFKNSFDTDTKK |
| | KKFDAIDNAKIIEYFEATNKAKKIEKLEEILKGIDKLSLKTLIQDDILLKFL |
| | LLFFTFLPQEIKGEFLGFIKKYYHDITSLDEDTKDKDDEITELPRSLKLKIFS |
| | KNIRKLSILKHSLSYQIKYNKKESSYYEAGNVFNKMFKKQAISHNLEEFG |
| | KSIYLPMLKYYSALYKLINDFEIYALYKDMDTSETLSQQVDKQEYKRNE |
| | YFNFETLLRKKFGNDIEKVLVTYRNKIAHLDFNFLYDKPINKFISLYKSRE |
| | KIVNYIKNHDIQAVLKYDAVNDFVMKVIQLRTKLKVYADKEQTIESMIQ |
| | NTQNPNGFYNIYKVKAVENINRHLLKVIGYTESEKAVEEKIRAGNTSKS |
| SEQ ID NO: | MIKNPSNRYALPKVIISKIDNQNILEFKIKYKKLSKLDIVKVKSMHYDDR |
| 95 | AIIFDEVIVNDGLIDVEYRDNHKTIFVKVGNKSYSISGQKVGGKERLLEN |
| | RVSKTKVQLELKDKATNRVSKTERELIVDDNIKIYSQIVGRDVKTTKDIY |
| | LIKRFLAYRSDLLFYYGFVNNFFHVANNRSEFWKIDFNDSNNSKLIEYFK |
| | FTINDHLKNDENYLKDYISDNEKLKNDLIKVKNSFEKIRHALMHFDYDFF |
| | VKLFNGEDVGLELDIEFLDIMIDKLDKLNIDTKKEFIDDEKITIFGEELSLA |
| | KLYRFYAHTAINRVAFNKLINSFIIENGVENQSLKEYFNQQAGGIAYEIDI |
| | HQNREYKNLYNEHKKLVSRVLSISDGQEIAILNQKIAKLKDQMKQITKA |
| | NSIKRLEYKLRLALGFIYTEYENYEEFKNNFDTDIKNGRFTPKDNDGNKR |
| | AFDSRELEQLKGYYEATIQTQKPKTDEKIEEVSKKIDRLSLKSLIADDILL |
| | KFILLMFTFMPQELKGEFLGFIKKYYHDTKHIDQDTISDSDDTIETLSIGLK |
| | LKILDKNIRSLSILKHSLSFQTKYNKKDRNYYEDGNIHGKFFKKLGISHNQ |
| | EEFNKSVYAPLFRYYSALYKLINDFEIYTLSLHIVGSETLTDQVNKSQFLS |
| | GRYFNFRKLLTQSYHINNNSTHSTIFNAVINMRNDISHLSYEPLFDCPLNG |
| | KKSYKRKIRNQFKTINIKPLVESRKIIIDFITLQTDMQKVLGYDAVNDFTM |
| | KIVQLRTRLKAYANKEQTIQKMITEAKTPNDFYNIYKVQGVEEINKYLLE |
| | VIGETQAEKEIREKIERGNIANF |
| SEQ ID NO: | MKKSIFDQFVNQYALSKTLRFELKPVGETGRMLEEAKVFAKDETIKKKY |
| 96 | EATKPFFNKLHREFVEEALNEVELAGLPEYFEIFKYWKRYKKKFEKDLQ |
| | KKEKELRKSVVGFFNAQAKEWAKKYETLGVKKKDVGLLFEENVFAILK |
| | ERYGNEEGSQIVDESTGKDVSIFDSWKGFTGYFIKFQETRKNFYKDDGTA |
| | TALATRIIDQNLKRFCDNLLIFESIRDKIDFSEVEQTMGNSIDKVFSVIFYSS |
| | CLLQEGIDFYNCVLGGETLPNGEKRQGINELINLYRQKTSEKVPFLKLLD |
| | KQILSEKEKFMDEIENDEALLDTLKIFRKSAEEKTTLLKNIFGDFVMNQG |
| | KYDLAQIYISRESLNTISRKWTSETDIFEDSLYEVLKKSKIVSASVKKKDG |
| | GYAFPEFIALIYVKSALEQIPTEKFWKERYYKNIGDVLNKGFLNGKEGV |
| | WLQFLLIFDFEFNSLFEREIIDENGDKKVAGYNLFAKGFDDLLNNFKYDQ |
| | KAKVVIKDFADEVLHIYQMGKYFAIEKKRSWLADYDIDSFYTDPEKGYL |
| | KFYENAYEEIIQVYNKLRNYLTKKPYSEDKWKLNFENPTLADGWDKNK |
| | EADNSTVILKKDGRYYLGLMARGRNKLFDDRNLPKILEGVENGKYEKV |
| | VYKYFPDQAKMFPKVCFSTKGLEFFQPSEEVITIYKNSEFKKGYTFNVRS |
| | MQRLIDFYKDCLVRYEGWQCYDFRNLRKTEDYRKNIEEFFSDVAMDGY |
| | KISFQDVSESYIKEKNQNGDLYLFEIKNKDWNEGANGKKNLHTIYFESLF |
| | SADNIAMNFPVKLNGQAEIFYRPRTEGLEKERIITKKGNVLEKGDKAFHK |
| | RRYTENKVFFHVPITLNRTKKNPFQFNAKINDFLAKNSDINVIGVDRGEK |
| | QLAYFSVISQRGKILDRGSLNVINGVNYAEKLEEKARGREQARKDWQQI |
| | EGIKDLKKGYISQVVRKLADLAIQYNAIIVFEDLNMRFKQIRGGIEKSVY |
| | QQLEKALIDKLTFLVEKEEKDVEKAGHLLKAYQLAAPFETFQKMGKQT |
| | GIVFYTQAAYTSRIDPVTGWRPHLYLKYSSAEKAKADLLKFKKIKFVDG |
| | RFEFTYDIKSFREQKEHPKATVWTVCSCVERFRWNRYLNSNKGGYDHY |
| | SDVTKFLVELFQEYGIDFERGDIVGQIEVLETKGNEKFFKNFVFFFNLICQI |
| | RNTNASELAKKDGKDDFILSPVEPFFDSRNSEKFGEDLPKNGDDNGAFNI |
| | ARKGLVIMDKITKFADENGGCEKMKWGDLYVSNVEWDNFVANK |
| SEQ ID NO: | MFNNFIKKYSLQKTLRFELKPVGETADYIEDFKSEYLKDTVLKDEQRAK |
| 97 | DYQEIKTLIDDYHREYIEECLREPVDKKTGEILDFTQDLEDAFSYYQKLK |
| | ENPTENRVGWEKEQESLRKKLVTSFVGNDGLFKKEFITRDLPEWLQKKG |
| | LWGEYKDTVENFKKFTTYFSGFHENRKNMYTAEAQSTAIANRLMNDNL |
| | PKFFNNYLAYQTIKEKHPDLVFRLDDALLQAAGVEHLDEAFQPRYFSRL |
| | FAQSGITAFNELIGGRTTENGEKIQGLNEQINLYRQQNPEKAKGFPRFMP |
| | LFKQILSDRETHSFLPDAFENDKELLQALRDYVDAATSEEGMISQLNKA |
| | MNQFVTADLKRVYIKSAALTSLSQELFHFFGVISDAIAWYAEKRLSPKKA |
| | QESFLKQEVYAIEELNQAVVGYIDQLEDQSELQQLLVDLPDPQKPVSSFI |
| | LTHWQKSQEPLQAVIAKVEPLFELEELSKNKRAPKHDKDQGGEGFQQV |
| | DAIKNMLDAFMEVSHAIKPLYLVKGRKAIDMPDVDTGFYADFAEAYSA |
| | YEQVTVSLYNKTRNHLSKKPFSKDKIKINFDAPTLLNGWDLNKESDNKSI |
| | ILRKDGNFYLAIMHPKHTKVFDCYSASEAAGKCYEKMNYKLLSGANKM |
| | LPKVFFSKKGIETFSPPQEILDLYKNNEHKKGATFKLESCHKLIDFFKRNIP |
| | KYKVHPTDNFGWDVFGFHFSPTSSYGDLSGFYREVEAQGYKLWFSDVS |
| | EAYINKCVEEGKLFLFQIYNKDFSPNSTGKPNLHTLYWKGLFEPENLKDV |
| | VLKLNGEAEIFYRKHSIKHEDKTIHRAKDPIANKNADNPKKQSVFDYDII |
| | KDKRYTQDKFFFHVPISLNFKSQGVVRFNDKINGLLAAQDDVHVIGIDR |
| | GERHLLYYTVVNGKGEVVEQGSLNQVATDQGYVVDYQQKLHAKEKER |
| | DQARKNWSTIENIKELKAGYLSQVVHKLAQLIVKHNAIVCLEDLNFGFK |
| | RGRFKVEKQVYQKFEKALIDKLNYLVFKERGATQAGGYLNAYQLAAPF |
| | ESFEKLGKQTGILYYVRSDYTSKIDPATGFVDFLKPKYESMAKSKVFFES |
| | FERIQWNQAKGYFEFEFDYKKMCPSRKFGDYRTRWVVCTFGDTRYQNR |
| | RNKSSGQWETETIDVTAQLKALFAAYGITYNQEDNIKDAIAAVKYTKFY |
| | KQLYWLLRLTLSLRHSVTGTDEDFILSPVADENGVFFDSRKATDKQPKD |
| | ADANGAYHIALKGLWNLQQIRQHDWNVEKPKKLNLAMKNEEWFGFAQ |
| | KKKFRA |
| SEQ ID NO: | MIKNPSNRYALPKVIISKIDNQNILEFKIKYKKLSKLDIVKVKSMHYDDR |
| 98 | AIIFDEVIVNDGLIDVEYRDNHKTIFVKVGNKSYSISGQKVGGKERLLEN |
| | RVSKTKVQLELKDKATNRVSKTERELIVDDNIKIYSQIVGRDVKTTKDIY |
| | LIKRFLAYRSDLLFYYGFVNNFFHVANNRSEFWKIDFNDSNNSKLIEYFK |
| | FTINDHLKNDENYLKDYISDNEKLKNDLIKVKNSFEKIRHALMHFDYDFF |
| | VKLFNGEDVGLELDIEFLDIMIDKLDKLNIDTKKEFIDDEKITIFGEELSLA |
| | KLYRFYAHTAINRVAFNKLINSFIIENGVENQSLKEYFNQQAGGIAYEIDI |
| | HQNREYKNLYNEHKKLVSRVLSISDGQEIAILNQKIAKLKDQMKQITKA |
| | NSIKRLEYKLRLALGFIYTEYENYEEFKNNFDTDIKNGRFTPKDNDGNKR |
| | AFDSRELEQLKGYYEATIQTQKPKTDEKIEEVSKKIDRLSLKSLIADDILL |
| | KFILLMFTFMPQELKGEFLGFIKKYYHDTKHIDQDTISDSDDTIETLSIGLK |
| | LKILDKNIRSLSILKHSLSFQTKYNKKDRNYYEDGNIHGKFFKKLGISHNQ |
| | EEFNKSVYAPLFRYYSALYKLINDFEIYTLSLHIVGSETLTDQVNKSQFLS |
| | GRYFNFRKLLTQSYHINNNSTHSTIFNAVINMRNDISHLSYEPLFDCPLNG |
| | KKSYKRKIRNQFKTINIKPLVESRKIIIDFITLQTDMQKVLGYDAVNDFTM |
| | KIVQLRTRLKAYANKEQTIQKMITEAKTPNDFYNIYKVQGVEEINKYLLE |
| | VIGETQAEKEIREKIERGNIANF |
| SEQ ID NO: | MIKNPSNRHSLPKVIISEVDHEKILEFKIKYEKLARLDRFEVKAMHYEGK |
| 99 | EIVFDEVLVNGGLIEVEYQDDNKTLFVKVGEKSYSIRGKKVGGKQRLLE |
| | DRVSKTKVQLELSDGVVDNKGNLRKSRTERELIVADNIKLYSQIVGREV |
| | TTTKEIYLVKRFLAYRSDLLFYYSFVDNFFKVAGNEKELWKINEDDATS |
| | AQFMGYIPFMVNDNLKNDNAYLKDYVRNDVQIKDDLKKVQTIFSALRH |
| | TLLHFNYEFFEKLFNGEDVGFDFDIGFLNLLIENIDKLNIDAKKEFIDNEKI |
| | RLFGENLSLAKVYRLYSDICVNRVGFNKFINSMLIKDGVENQVLKAEFN |
| | RKFGGNAYTIDIHSNQEYKRIYNEHKKLVIKVSTLKDGQAIRRGNKKISE |
| | LKEQMKSMTKKNSLARLECKMRLAFGFLYGEYNNYKAFKNNFDTNIKN |
| | SQFDVNDVEKSKAYFLSTYERRKPRTREKLEKVAKDIESLELKTVIANDT |
| | LLKFILLMFVFMPQELKGDFLGFVKKYYHDVHSIDDDTKEQEEDVVEA |
| | MSTSLKLKILGRNIRSLTLFKYALSSQVNYNSTDNIFYVEGNRYGKIYKK |
| | LGISHNQEEFDKTLVVPLLRYYSSLFKLMNDFEIYSLAKANPTAVSLQEL |
| | VDDETSPYKQGNYFNFNKMLRDIYGLTSDEIKSGQVVFMRNKIAHFDTE |
| | VLLSKPLLGQTKMNLQRKDIVSFIEARGDIKELLGYDAINDFRMKVIHLR |
| | TKMRVYSDKLQTMMDLLRNAKTPNDFYNVYKVKGVESINKHLLEVLA |
| | QTAEERTVEKQIRDGNEKYDL |
| SEQ ID NO: | MEEKMLKSYDYFTKLYSLQKTLRFELKPIGKTLEHIKNSGIIESDETLEEQ |
| 100 | YAIVKNIIDKLHRKHIDEALSLVDFTKHLDTLKTFQELYLKRGKTDKEKE |
| | ELEKLSADLRKLIVSYLKGNVKEKTQHNLNPIKERFEILFGKELFTNEEFF |
| | LLAENEKEKKAIQAFKGFTTYFKGFQENRKNMYSEEGNSTSIAYRIINEN |
| | LPLFIENIARFQKVMSTIEKTTIKKLEQNLKTELKKHNLPGIFTIEYFNNVL |
| | TQEGISRYNTIIGGKTTHEGVKIQGLNEIINLYNQQSKDVKLPILKPLHKQI |
| | LSEEYSTSFKIKAFENDNEVLKAIDTFWNEHIEKSIHPVTGNKFNILSKIEN |
| | LCDQLQKYKDKDLEKLFIERKNLSTVSHQVYGQWNIIRDALRMHLEMN |
| | NKNIKEKDIDKYLDNDAFSWKEIKDSIKIYKEHVEDAKELNENGIIKYFS |
| | AMSINEEDDEKEYSISLIKNINEKYNNVKSILQEDRTGKSDLHQDKEKVGI |
| | IKEFLDSLKQLQWFLRLLYVTVPLDEKDYEFYNELEVYYEALLPLNSLY |
| | NKVRNYMTRKPYSVEKFKLNFNSPTLLDGWDKNKETANLSIILRKNGKY |
| | YLGIMNKENNTIFEYYPGTKSNDYYEKMIYKLLPGPNKMLPKVFFSKKG |
| | LEYYNPPKEILNIYEKGEFKKDKSGNFKKESLHTLIDFYKEAIAKNEDWE |
| | VFNFKFKNTKEYEDISQFYRDVEEQGYLITFEKVDANYVDKLVKEGKLY |
| | LFQIYNKDFSENKKSKGNPNLHTIYWKGLYDSENLKNVVYKLNGEAEVF |
| | YRKKSIDYPEEIYNHGHHKEELLGKFNYPIIKDRRYTQDKFLFHVPITMNF |
| | ISKEEKRVNQLACEYLSATKEDVHIIGIDRGERHLLYLSLIDKEGNIKKQL |
| | SLNTIKNENYDKEIDYRVKLDEKEKKRDEARKNWDVIENIKELKEGYMS |
| | QVIHIIAKMMVEEKAILIMEDLNIGFKRGRFKVEKQVYQKFEKMLIDKLN |
| | YLVFKNKNPLEPGGSLNAYQLTSKFDSFKKLGKQSGFIFYVPSAYTSKID |
| | PTTGFYNFIQVDVPNLEKGKEFFSKFEKIIYNTKEDYFEFHCKYGKFVSEP |
| | KNKDNDRKTKESLTYYNAIKDTVWVVCSTNHERYKIVRNKAGYYESHP |
| | VDVTKNLKDIFSQANINYNEGKDIKPIIIESNNAKLLKSIAEQLKLILAMR |
| | YNNGKHGDDEKDYILSPVKNKQGKFFCTLDGNQTLPINADANGAYNIAL |
| | KGLLLIEKIKKQQGKIKDLYISNLEWFMFMMSR |
| SEQ ID NO: | MEKSLNDFIGLYSVSKTLRFELKPVSETLENIKKFHFLEEDKKKANDYKD |
| 101 | VKKIIDNYHKYFIDDVLKNASFNWKKLEEAIREYNKNKSDDSALVAEQK |
| | KLGDAILKLFTSDKRYKALTAATPKELFESILPDWFGEQCNQDLNKAAL |
| | KTFQKFTSYFTGFQENRKNVYSAEAIPTAVPYRIVNDNFPKFLQNVLIFKT |
| | IQEKCPQIIDEVEKELSSYLGKEKLAGIFTLESFNKYLGQGGKENQRGIDF |
| | YNQIIGGVVEKEGGINLRGVNQFLNLYWQQHPDFTKEDRRIKMVPLYKQ |
| | ILSDRSSLSFKIESIENDEELKNALLECADKLELKNDEKKSIFEEVCDLFSS |
| | VKNLDLSGIYINRKDINSVSRILTGDWSWLQSRMNVYAEEKFTTKAEKA |
| | RWQKSLDDEGENKSKGFYSLTDLNEVLEYSSENVAETDIRITDYFEHRCR |
| | YYVDKETEMFVQGSELVALSLQEMCDDILKKRKAMNTVLENLSSENKL |
| | REKTDDVAVIKEYLDAVQELLHRIKPLKVNGVGDSTFYSVYDSIYSALSE |
| | VISVYNKTRNYITKKAASPEKYKLNFDNPTLADGWDLNKEQANTSVILR |
| | KDGMFYLGIMNPKNKPKFAEKYDCGNESCYEKMIYKQFDATKQIPKCST |
| | QKKEVQKYFLSGATEPYILNDKKSFKSELIITKDIWFMNNHVWDGEKFV |
| | PKRDNETRPKKFQIGYFKQTGDFDGYKNALSNWISFCKNFLQSYLSATV |
| | YDYNFKNSEEYEGLDEFYNYLNATCYKLNFINIPETEINKMVSEGKLYLF |
| | QIYNKDFASGSTGMPNMHTLYWKNLFSDENLKNVCLKLNGEAELFYRP |
| | AGIKEPVIHKEGSYLVNRTTEDGESIPEKIYFEIYKNANGKLEKLSDEAQN |
| | YISNHEVVIKKAGHEIIKDRHYTEPKFLFHVPLTINFKASGNSYSINENVR |
| | KFLKNNPDVNIIGLDRGERHLIYLSLINQKGEIIKQFTFNEVERNKNGRTIK |
| | VNYHEKLDQREKERDAARKSWQAIGKIAELKEGYLSAVIHQLTKLMVE |
| | YNAVVVMEDLNFGFKRGRFHVEKQVYQKFEHILIDKSNYLVFKDRGLN |
| | EPGGVLNGYQIAGQFESFQKLGKQSGMLFYVPAGYTSKIDPKTGFVSMM |
| | NFKDLTNVHKKRDFFSKFDNIHYDEANGSFVFTFDYKKFDGKAKEEMK |
| | LTKWSVYSRDKRIVYFAKTKSYEDVLPTEKLQKIFESNGIDYKSGNNIQD |
| | SVMAIGADLKEGAKPSKEISDFWDGLLSNFKLILQMRNSNARTGEDYIIS |
| | PVMADDGTFFDSREEFKKGEDAKLPLDADANGAYHIALKGLSLINKINL |
| | SKDEELKKFDMKISNADWFKFAQEKNYAK |
| SEQ ID NO: | MNTQKKEFNPKSFKDFTNLYSLNKTLRFSLTPNKKTAEILEFNKQKEVKC |
| 102 | FSNDRKIAGAYQEIKKYLNKLHQEFIQEAMKFFAFSEEELKGFEKEYLNL |
| | LNFTDKDNFKKKNKIRNEYEQERKILTIKIATYFSKFKSEKYQSFNLANIT |
| | GKKVFSILEQKYKEDKKTLKIIHIFKYKPTKDEKKEGEAVNFSTYLTGFN |
| | ENRKNFYKSEDKAGQFATRTIDNLAQFIKNKKLFEDKYQKNYSKIGILDE |
| | QIKIFNLDYFNNLFLQEGLDEYNGILGNNKGEENKSNEGINQKINIFKQKE |
| | KARLKKEKENFNKSDFPLFKELYKQIGSIRKENDVYVEIKTDKELVEELN |
| | NFPKNVENYLKDIQSFYKTFFEKLQNEEYELDKIYLPKSVGTYFSYIAFSD |
| | WNKLAFIYNKRYKNEKIKIVEGGDVNVQYRSLEVLKNRIDELKDEDNLN |
| | FNKFFIDKLKFNEAKKENNWQNFWFCIEYYINSQFIGGEKNILNKEKNEY |
| | EILPFGSLKELKEKYFEAVKKYKEKMVDTESGLTDDEEKEIKETLKNYLD |
| | RIKEIERIAKYFDLKKSFEEIKQEDLDSNFYGEYQKVVDKTNELKIYQYYS |
| | EFRNYLTQNNSVEEKIKLNFNSGLLLDGWDLNKEKVKFSIIFQENGKYYL |
| | GIINKEKDKTILDKDKHPEIFTKNSDFRKMEYKLFPSPSKMLPKISFSETA |
| | KKGDEDVGWSEEIQKIKDEFAEFQEYKKKSKDNWKDEFNRGKLNKLID |
| | YYKQVLEKHSEGYMNTYNFELKDSSKYKNLGEFNDDIARQNYKVKFVG |
| | IDKNYIDEKVANGELFLFQIYNKDFSEDKKEGSTNNLETIYFKELFSKENL |
| | ENPVFKLSGGAEMFFRNKIEKKKEKKKLDKDGKPMISKKGEKVVDKRR |
| | FSENKILFHLPIEINYGKGKMPNFNKKINEYISKNPENIKIIGIDRGEKHLLY |
| | YSIIDQNGNNIESMSLNAVDEFGNFVNPEKLEEYEIDNNGKKERRWKYIV |
| | NDKEIKVTNYQRKLDELEKERQKSRQSWQNINKIKNLKKGYISFVVKKI |
| | VDLAIENNAIIILEDLNFGFKSFRQKIEKNVYQQFEKALIDKLGFVVDKQK |
| | QNQRFAPQLSAPFESFQKIGKQTGIVYYVLANNTSKVCPSCQWIKNFYLK |
| | YEKKNTIFNLQKNQKLKVFFEQEKNRFRFEYQMSKEYISVYSDVDRQRY |
| | DKTKNQNKGGYLEYKNSNQKEIIDKDGVIQKQSITLQLKELFKENHIDLE |
| | KEILKQLDNKKEKNSGYTGVYNKFIYLFNLILQIRNAISFREKDYIQCPSC |
| | HFDTRKENYLKINDGDGNGAYNIALRGLYLLKGKNGIINNLEKIKLIFSN |
| | NDYFQWAKKLKNKK |
| SEQ ID NO: | MQNKQSFADFTNLYSLSKTLRFELKPIGQTQAMLDENKIFEVDENRKKA |
| 103 | YDKTKPYFDRLHREFINESLSNAQLKGISEYFETFKQFRSNQNNKDLKELI |
| | NKQQKFLRHQIVTLFDENGKHWATTKYAHLKIKKKNLDILFDEQVFYIL |
| | KERYGSEKETQLVDKETGAVTSIFDNWKGFTGYFTKFFETRKNFYKSDG |
| | TSTALATRIIDQNLNRFFDNLETFHKIKDKIDVKEVEIFFKLKADNVFSIDF |
| | YNQCLLQNGIDKYNDFLGGQTLENGEKQKGINEIINKYRQDNKDQKLPF |
| | LKKLDKQILSEKDRFINEIESKEEFFQVLTEFYQSATVKVTIIKTLLNDFVH |
| | NTDKYKLEKIYLTKEAFNTIANKWTDETQIFEDNLDLVLKNKKITAKQD |
| | FIPLAYIKEALEVIEKDRKFFKDRYYNDPQIGFFPDQSYWEQFLAILNFEF |
| | MTHFQRVAKDKITGKKIELGYFVFEKRIKELLDSDPSLNSQSKIIIKEFADE |
| | VLHIFQMAKYFALEKKREWKGDYYQLDDQFYNHIDYGFKDQFYENAY |
| | EKIVQPYNKIRNYLTKKPYSDVKWKLNFGNPTLANGWDKNKEADNTAV |
| | ILKKDGNYYLGVMKKGKNKIFSDQNKEKYKAYNSAYYEKLVYKLFPDP |
| | SKMFPKVCFSKKGLNFFQPSEEILRIYKNNEFKKGNTFSISSMQKLIAFYID |
| | CLGLYEGWKHYEFKNIKDVRQYKENIGEFYADVAESGYKLWFEKISEEY |
| | ITQKNQLGELFLFQIYNKDFAKKTTGRKNLHTIYFEELFSQTNIDNNFPFK |
| | LNGQAELFYRPKSLEKIEEKRNFKRSIVNKKRYTQNKIFFHVPITLNRTSE |
| | NIGRFNVRVNNFLANNSNVNIVGVDRGEKNLAYYSIIKQNGEVLKSGSL |
| | NIINGVDYHALLTDRAQRREQERRNWQDVESIKDLKRGYISQVVHELVS |
| | LAIKYNAIIVMEDLNMRFKQIRGGIEKSTYQQLEKALIEKLNFLVNKEET |
| | DSNQAGNLLNAYQLTAPFKTFKDMGKQTGIIFYTQASYTSKIDPLTGWR |
| | PNIYLRYSNAKQAKADILMFTNIYFSEKKDRFEFTYDLEKIDDKRKDLPIK |
| | TEWTVCSNVERFSWEKSLNNNKGGYVHYPIQDSNGEESITSKLKKLFMD |
| | FGIDLTDIKTQIESLDTNKKDNANFFRKFIFYFQLICQIRNTQVNKSDDGN |
| | DFIFSPVEPFFDSRFADKFRKNLPKNGDENGAYNIARKGLIILHKISDYFV |
| | KEGSTDKISWKDLSISQTEWDNFTTDK |
| SEQ ID NO: | MKKEKEFKSFGDFTNLYEISKTLRFELKPVENTQTMLDEADVFGKDKVI |
| 104 | KDKYTKTKPFIDKLHREFVDESLKDVSLSGLKKYSEVLENWKKNKKDK |
| | DIVKELKKEEERLRKEVVEFFDNTAKKWANEKYKELGLKKKDIGILFEE |
| | SVFDLLKEKYGEEQDSFLKEEKGDFLKNEKGEKVSIFDEWKGFVGYFTK |
| | FQETRKNFYKNDGTETALATRIIDQNLKRFCDNIDDFKKIKNKIDFSEVEK |
| | NFNKTADVFSLDFYNQCLLQKGIDSYNEFIGGKTLENGKKLKGVNELVN |
| | EYRQKNKNEKVSFLKLLDKQILSEKEKLSFGIENDEQLLVVLNSFYETAE |
| | EKTKILRTLFGDFVEHNENYDLDKTYISKVAFNTISHKWTNETHKFEELL |
| | YGAMKEDKPIGLNYDKKEDSYKFPDFIALGYLKKCLNNLDCDTKFWKE |
| | KYYENNADKKDKDKGFLTGGQNAWDQFLQIFIFEFNQLFNSEAFDNKG |
| | KEIKIGYDNFRKDFEEIINQKDFKNDENLKIAIKNFADSVLWIYQMAKYF |
| | AIEKKRGWDDDFELSEFYTNPSNGYSLFYDRAYEEIVQKYNDLRNYLTK |
| | KPYKEDKWKLNFENPTLANGFDKNKESDNSTVILRKKRKYYLGLMKKG |
| | NNKIFEDRNKAEFIRNIESGAYEKMAYKYLPDVAKMIPKCSTQLNEAKN |
| | HFRNSADDLEIKKSFSNPLKITKRIFDLNNIQYDKTNVSKKISGDNKGIKIF |
| | QKEYYKISGDFDVYKSALNDWIDFCKDFLSKYDSTKDFDFSILRKTKDY |
| | KSLDEFYVDVAKITYKISFTPVSESYIDQKNKNGELYLFEIYNQDFAKGK |
| | MGAKNLHTLYFENVFSPENISKNFPIKLNGNAELFFRPKSIESKKEKRNFV |
| | REIVNKKRYSEDKIFFHCPITLNRETGSIYRENNYVNNFLSENNINIIGVDR |
| | GEKHLAYYSVIDKNGVKIGGGSFNEINKVDYAKKLEERAGEREQSRKD |
| | WQVVEGIKDLKKGYISQVVRELADLAIKHNAIIVLEDLNMRFKQIRGGIE |
| | KSIYQQLEKALIDKLSFLVEKGEKDPNQAGHILKAYQLAAPFTSFKDMG |
| | KQTGIVFYTQASYTSKTCPNCGFRKNNNKFYFENNIGKAQDALKKLKTF |
| | EYDSENKCFGLSYCLSDFANKEEVEKNKNKKRNNAPYSDIEKKDCFELS |
| | TKDAVRYRWHDKNTERGKTFFEGESVYEEKEEKEIGQTKRGLVKEYDIS |
| | KCLIGLFEKTGLDYKQNLLDKINSGKFDGTFYKNLFNYLNLLFEIRNSISG |
| | TEIDYISCPECQFHTDKSKTIKNGDDNGSYNIARKGMIILDKIKQFKKENG |
| | SLDKMGWGELFIDLEEWDKFAQKKNNNIIDK |
| SEQ ID NO: | MKYTDFTGIYSVSKTLRFELIPQGSTVENMKREGILNNDMHRADSYKEM |
| 105 | KKLIDEYHKAFIERCLSDFSLKYDDTGKHDSLEEYFFYYEQKRNDKTKKI |
| | FEDIQVALRKQISKRFTGDTAFKRLFKKELIKEDLPSFVKNDPVKTELIKE |
| | FSDFTTYFQEFHKNRKNMYTSDAKSTAIAYRIINENLPKFIDNINAFDIVA |
| | KVPEMQEHFKTIADELRSHLQVGNDIDKMFNLQFFNKVLTQSQLDVYN |
| | AVIGGKSEGNKKIQGINEYINLYNQQHKKARLPMLKLLYKQILSDRVAIS |
| | WLQDEFDNDQDMLDTIEAFYNKLNSNETGVLGEGKLKQILMGLDGYNL |
| | DGVFMRNDLQLSEVSQRLCGGWNIIKDAMTSDLKRSVQKKKKETDADF |
| | EERVSKLFSAQNSFSIAYINQCLGQAGIRCKIQDYFACLGAKEGENEAET |
| | TPDIFDQIAEAYHGAAPILNARPSSHNLAQDIEKVKAIKALLDALKRLQRF |
| | VKPLLGRGDEGDKDNFFYGDFMPIWEVLDQLTPLYNKVRNRMTRKPYS |
| | QEKIKLNFENSTLLNGWDLNKEHDNTSVILRREGLYYLGIMNKNYNKIF |
| | DANNVETIGDCYEKMIYKLLPGPNKMLPKVFFSKSRVQEFSPSKKILEIW |
| | ESKSFKKGDNFNLDDCHALIDFYKDSIAKHPDWNKFNFKFSDTQSYTNIS |
| | DFYRDVNQQGYSLSFTKVSVDYVNRMVDEGKLYLFQIYNKDFSPQSKG |
| | TPNMHTLYWRMLFDERNLHNVIYKLNGEAEVFYRKASLRCDRPTHPAH |
| | QPITCKNENDSKRVCVFDYDIIKNRRYTVDKFMFHVPITINYKCTGSDNI |
| | NQQVCDYLRSAGDDTHIIGIDRGERNLLYLVIIDQHGTIKEQFSLNEIVNE |
| | YKGNTYCTNYHSLLEEKEAGNKKARQDWQTIESIKELKEGYLSQVIHKIS |
| | MLMQRYHAIVVLEDLNGSFMRSRQKVEKQVYQKFEHMLINKLNYLVN |
| | KQYDATEPGGLLHALQLTSRMDSFKKLGKQSGFLFYIPAWNTSKIDPVT |
| | GFVNLFDTRYCNEAKAKEFFEKFDDISYNDERDWFEFSFDYRHFTNKPT |
| | GTRTQWTLCTQGTRVRTFRNPEKSNHWDNEEFDLTQAFKDLFNKYGIDI |
| | ASGLKARIVNGQLTKETSAVKDFYESLLKLLKLTLQMRNSVTGTDIDYL |
| | VSPVADKDGIFFDSRTCGSLLPANADANGAFNIARKGLMLLRQIQQSSID |
| | AEKIQLAPIKNEDWLEFAQEKPYL |
| SEQ ID NO: | MEKEITELTKIRREFPNKKFSSTDMKKAGKLLKAEGPDAVRDFLNSCQEI |
| 106 | IGDFKPPVKTNIVSISRPFEEWPVSMVGRAIQEYYFSLTKEELESVHPGTSS |
| | EDHKSFFNITGLSNYNYTSVQGLNLIFKNAKAIYDGTLVKANNKNKKLE |
| | KKFNEINHKRSLEGLPIITPDFEEPFDENGHLNNPPGINRNIYGYQGCAAK |
| | VFVPSKHKMVSLPKEYEGYNRDPNLSLAGFRNRLEIPEGEPGHVPWFQR |
| | MDIPEGQIGHVNKIQRFNFVHGKNSGKVKFSDKTGRVKRYHHSKYKDA |
| | TKPYKFLEESKKVSALDSILAIITIGDDWVVFDIRGLYRNVFYRELAQKGL |
| | TAVQLLDLFTGDPVIDPKKGVVTFSYKEGVVPVFSQKIVPRFKSRDTLEK |
| | LTSQGPVALLSVDLGQNEPVAARVCSLKNINDKITLDNSCRISFLDDYKK |
| | QIKDYRDSLDELEIKIRLEAINSLETNQQVEIRDLDVFSADRAKANTVDM |
| | FDIDPNLISWDSMSDARVSTQISDLYLKNGGDESRVYFEINNKRIKRSDY |
| | NISQLVRPKLSDSTRKNLNDSIWKLKRTSEEYLKLSKRKLELSRAVVNYT |
| | IRQSKLLSGINDIVIILEDLDVKKKFNGRGIRDIGWDNFFSSRKENRWFIPA |
| | FHKAFSELSSNRGLCVIEVNPAWTSATCPDCGFCSKENRDGINFTCRKCG |
| | VSYHADIDVATLNIARVAVLGKPMSGPADRERLGDTKKPRVARSRKTM |
| | KRKDISNSTVEAMVTA |
| SEQ ID NO: | MIKPTVSQFLTPGFKLIRNHSRTAGLKLKNEGEEACKKFVRENEIPKDEC |
| 107 | PNFQGGPAIANIIAKSREFTEWEIYQSSLAIQEVIFTLPKDKLPEPILKEEW |
| | RAQWLSEHGLDTVPYKEAAGLNLIIKNAVNTYKGVQVKVDNKNKNNL |
| | AKINRKNEIAKLNGEQEISFEEIKAFDDKGYLLQKPSPNKSIYCYQSVSPK |
| | PFITSKYHNVNLPEEYIGYYRKSNEPIVSPYQFDRLRIPIGEPGYVPKWQY |
| | TFLSKKENKRRKLSKRIKNVSPILGIICIKKDWCVFDMRGLLRTNHWKKY |
| | HKPTDSINDLFDYFTGDPVIDTKANVVRFRYKMENGIVNYKPVREKKGK |
| | ELLENICDQNGSCKLATVDVGQNNPVAIGLFELKKVNGELTKTLISRHPT |
| | PIDFCNKITAYRERYDKLESSIKLDAIKQLTSEQKIEVDNYNNNFTPQNTK |
| | QIVCSKLNINPNDLPWDKMISGTHFISEKAQVSNKSEIYFTSTDKGKTKD |
| | VMKSDYKWFQDYKPKLSKEVRDALSDIEWRLRRESLEFNKLSKSREQD |
| | ARQLANWISSMCDVIGIENLVKKNNFFGGSGKREPGWDNFYKPKKENR |
| | WWINAIHKALTELSQNKGKRVILLPAMRTSITCPKCKYCDSKNRNGEKF |
| | NCLKCGIELNADIDVATENLATVAITAQSMPKPTCERSGDAKKPVRARK |
| | AKAPEFHDKLAPSYTVVLREAV |
| SEQ ID NO: | MEKEITELTKIRREFPNKKFSSTDMKKAGKLLKAEGPDAVRDFLNSCQEI |
| 108 | IGDFKPPVKTNIVSISRPFEEWPVSMVGRAIQEYYFSLTKEELESVHPGTSS |
| | EDHKSFFNITGLSNYNYTSVQGLNLIFKNAKAIYDGTLVKANNKNKKLE |
| | KKFNEINHKRSLEGLPIITPDFEEPFDENGHLNNPPGINRNIYGYQGCAAK |
| | VFVPSKHKMVSLPKEYEGYNRDPNLSLAGFRNRLEIPEGEPGHVPWFQR |
| | MDIPEGQIGHVNKIQRFNFVHGKNSGKVKFSDKTGRVKRYHHSKYKDA |
| | TKPYKFLEESKKVSALDSILAHITIGDDWVVFDIRGLYRNVFYRELAQKGL |
| | TAVQLLDLFTGDPVIDPKKGVVTFSYKEGVVPVFSQKIVPRFKSRDTLEK |
| | LTSQGPVALLSVDLGQNEPVAARVCSLKNINDKITLDNSCRISFLDDYKK |
| | QIKDYRDSLDELEIKIRLEAINSLETNQQVEIRDLDVFSADRAKANTVDM |
| | FDIDPNLISWDSMSDARVSTQISDLYLKNGGDESRVYFEINNKRIKRSDY |
| | NISQLVRPKLSDSTRKNLNDSIWKLKRTSEEYLKLSKRKLELSRAVVNYT |
| | IRQSKLLSGINDIVIILEDLDVKKKFNGRGIRDIGWDNFFSSRKENRWFIPA |
| | FHKTFSELSSNRGLCVIEVNPAWTSATCPDCGFCSKENRDGINFTCRKCG |
| | VSYHADIDVATLNIARVAVLGKPMSGPADRERLGDTKKPRVARSRKTM |
| | KRKDISNSTVEAMVTA |
| SEQ ID NO: | VPDKKETPLVALCKKSFPGLRFKKHDSRQAGRILKSKGEGAAVAFLEGK |
| 109 | GGTTQPNFKPPVKCNIVAMSRPLEEWPIYKASVVIQKYVYAQSYEEFKA |
| | TDPGKSEAGLRAWLKATRVDTDGYFNVQGLNLIFQNARATYEGVLKKV |
| | ENRNSKKVAKIEQRNEHRAERGLPLLTLDEPETALDETGHLRHRPGINCS |
| | VFGYQHMKLKPYVPGSIPGVTGYSRDPSTPIAACGVDRLEIPEGQPGYVP |
| | PWDRENLSVKKHRRKRASWARSRGGAIDDNMLLAVVRVADDWALLDL |
| | RGLLRNTQYRKLLDRSVPVTIESLLNLVINDPTLSVVKKPGKPVRYTATL |
| | IYKQGVVPVVKAKVVKGSYVSKMLDDTTETFSLVGVDLGVNNLIAANA |
| | LRIRPGKCVERLQAFTLPEQTVEDFFRFRKAYDKHQENLRLAAVRSLTAE |
| | QQAEVLALDTFGPEQAKMQVCGHLGLSVDEVPWDKVNSRSSILSDLAK |
| | ERGVDDTLYMFPFFKGKGKKRKTEIRKRWDVNWAQHFRPQLTSETRKA |
| | LNEAKWEAERNSSKYHQLSIRKKELSRHCVNYVIRTAEKRAQCGKVIVA |
| | VEDLHHSFRRGGKGSRKSGWGGFFAAKQEGRWLMDALFGAFCDLAVH |
| | RGYRVIKVDPYNTSRTCPECGHCDKANRDRVNREAFICVCCGYRGNADI |
| | DVAAYNIAMVAITGVSLRKAARASVASTPLESLAAE |
| SEQ ID NO: | MPKPAVESEFSKVLKKHFPGERFRSSYMKRGGKILAAQGEEAVVAYLQ |
| 110 | GKSEEEPPNFQPPAKCHVVTKSRDFAEWPIMKASEAIQRYIYALSTTERA |
| | ACKPGKSSESHAAWFAATGVSNHGYSHVQGLNLIFDHTLGRYDGVLKK |
| | VQLRNEKARARLESINASRADEGLPEIKAEEEEVATNETGHLLQPPGINPS |
| | FYVYQTISPQAYRPRDEIVLPPEYAGYVRDPNAPIPLGVVRNRCDIQKGC |
| | PGYIPEWQREAGTAISPKTGKAVTVPGLSPKKNKRMRRYWRSEKEKAQ |
| | DALLVTVRIGTDWVVIDVRGLLRNARWRTIAPKDISLNALLDLFTGDPVI |
| | DVRRNIVTFTYTLDACGTYARKWTLKGKQTKATLDKLTATQTVALVAI |
| | DLGQTNPISAGISRVTQENGALQ |
| | CEPLDRFTLPDDLLKDISAYRIAWDRNEEELRARSVEALPEAQQAEVRAL |
| | DGVSKETARTQLCADFGLDPKRLPWDKMSSNTTFISEALLSNSVSRDQV |
| | FFTPAPKKGAKKKAPVEVMRKDRTWARAYKPRLSVEAQKLKNEALWA |
| | LKRTSPEYLKLSRRKEELCRRSINYVIEKTRRRTQCQIVIPVIEDLNVRFFH |
| | GSGKRLPGWDNFFTAKKENRWFIQGLHKAFSDLRTHRSFYVFEVRPERT |
| | SITCPKCGHCEVGNRDGEAFQCLSCGKTCNADLDVATHNLTQVALTGK |
| | TMPKREEPRDAQGTAPARKTKKASKSKAPPAEREDQTPAQEPSQTS |
| SEQ ID NO: | MSNKTTPPSPLSLLLRAHFPGLKFESQDYKIAGKKLRDGGPEAVISYLTG |
| 111 | KGQAKLKDVKPPAKAFVIAQSRPFIEWDLVRVSRQIQEKIFGIPATKGRP |
| | KQDGLSETAFNEAVASLEVDGKSKLNEETRAAFYEVLGLDAPSLHAQA |
| | QNALIKSAISIREGVLKKVENRNEKNLSKTKRRKEAGEEATFVEEKAHDE |
| | RGYLIHPPGVNQTIPGYQAVVIKSCPSDFIGLPSGCLAKESAEALTDYLPH |
| | DRMTIPKGQPGYVPEWQHPLLNRRKNRRRRDWYSASLNKPKATCSKRS |
| | GTPNRKNSRTDQIQSGRFKGAIPVLMRFQDEWVIIDIRGLLRNARYRKLL |
| | KEKSTIPDLLSLFTGDPSIDMRQGVCTFIYKAGQACSAKMVKTKNAPEIL |
| | SELTKSGPVVLVSIDLGQTNPIAAKVSRVTQLSDGQLSHETLLRELLSNDS |
| | SDGKEIARYRVASDRLRDKLANLAVERLSPEHKSEILRAKNDTPALCKA |
| | RVCAALGLNPEMIAWDKMTPYTEFLATAYLEKGGDRKVATLKPKNRPE |
| | MLRRDIKFKGTEGVRIEVSPEAAEAYREAQWDLQRTSPEYLRLSTWKQE |
| | LTKRILNQLRHKAAKSSQCEVVVMAFEDLNIKMMHGNGKWADGGWD |
| | AFFIKKRENRWFMQAFHKSLTELGAHKGVPTIEVTPHRTSITCTKCGHCD |
| | KANRDGERFACQKCGFVAHADLEIATDNIERVALTGKPMPKPESERSGD |
| | AKKSVGARKAAFKPEEDAEAAE |
| SEQ ID NO: | MSKTKELNDYQEALARRLPGVRHQKSVRRAARLVYDRQGEDAMVAFL |
| 112 | DGKEVDEPYTLQPPAKCHILAVSRPIEEWPIARVTMAVQEHVYALPVHE |
| | VEKSRPETTEGSRSAWFKNSGVSNHGVTHAQTLNAILKNAYNVYNGVIK |
| | KVENRNAKKRDSLAAKNKSRERKGLPHFKADPPELATDEQGYLLQPPSP |
| | NSSVYLVQQHLRTPQIDLPSGYTGPVVDPRSPIPSLIPIDRLAIPPGQPGYV |
| | PLHDREKLTSNKHRRMKLPKSLRAQGALPVCFRVFDDWAVVDGRGLLR |
| | HAQYRRLAPKNVSIAELLELYTGDPVIDIKRNLMTFRFAEAVVEVTARKI |
| | VEKYHNKYLLKLTEPKGKPVREIGLVSIDLNVQRLIALAIYRVHQTGESQ |
| | LALSPCLHREILPAKGLGDFDKYKSKFNQLTEEILTAAVQTLTSAQQEEY |
| | QRYVEESSHEAKADLCLKYSITPHELAWDKMTSSTQYISRWLRDHGWN |
| | ASDFTQITKGRKKVERLWSDSRWAQELKPKLSNETRRKLEDAKHDLQR |
| | ANPEWQRLAKRKQEYSRHLANTVLSMAREYTACETVVIAIENLPMKGG |
| | FVDGNGSRESGWDNFFTHKKENRWMIKDIHKALSDLAPNRGVHVLEVN |
| | PQYTSQTCPECGHRDKANRDPIQRERFCCTHCGAQRHADLEVATHNIAM |
| | VATTGKSLTGKSLAPQRLQEAAE |
| SEQ ID NO: | VAFLDGKEVDEPYTLQPPAKCHILAVSRPIEEWPIARVTMAVQEHVYAL |
| 113 | PVHEVEKSRPETTEGSRSAWFKNSGVSNHGVTHAQTLNAILKNAYNVY |
| | NGVIKKVENRNAKKRDSLAAKNKSRERKGLPHFKADPPELATDEQGYL |
| | LQPPSPNSSVYLVQQHLRTPQIDLPSGYTGPVVDPRSPIPSLIPIDRLAIPPG |
| | QPGYVPLHDREKLTSNKHRRMKLPKSLRAQGALPVCFRVFDDWAVVD |
| | GRGLLRHAQYRRLAPKNVSIAELLELYTGDPVIDIKRNLMTFRFAEAVVE |
| | VTARKIVEKYHNKYLLKLTEPKGKPVREIGLVSIDLNVQRLIALAIYRVH |
| | QTGESQLALSPCLHREILPAKGLGDFDKYKSKFNQLTEEILTAAVQTLTS |
| | AQQEEYQRYVEESSHEAKADLCLKYSITPHELAWDKMTSSTQYISRWLR |
| | DHGWNASDFTQITKGRKKVERLWSDSRWAQELKPKLSNETRRKLEDAK |
| | HDLQRANPEWQRLAKRKQEYSRHLANTVLSMAREYTACETVVIAIENLP |
| | MKGGFVDGNGSRESGWDNFFTHKKENRWMIKDIHKALSDLAPNRGVH |
| | VLEVNPQYTSQTCPECGHRDKANRDPIQRERFCCTHCGAQRHADLEVAT |
| | HNIAMVATTGKSLTGKSLAPQRLQ |
| SEQ ID NO: | MIKKPSNRHALPKVIISKVDNQNILEFKIKYKKLSRLDRVEIKTMHYDDR |
| 114 | AIVFDEVIINGGLIDVEYRDNHKTIFVKVGDKSYSISGQKVGGKERLLEN |
| | RISQTKVQLELKDEATNRVSKTERELIVDDNIKLYSQIVGRDVKTTKDIY |
| | LIKRFLGYRSDLLFYYGFVNNFFHVANNRPEFWKIDFNDNRNSKLIEYFIF |
| | TINDHLKNDENYLKDYISDRGQIVDDLENIKHIFSALRHGLMHFDYDFFE |
| | ALFNGEDIDIKMDNQGNTQPLSSLNIKFLDIMIDKLDKLNIDTKKEFIDAE |
| | KITIFGEELSLAKLYRFYAHTAINRVAFNKLINSFIIENGVENQSLKEYFNQ |
| | QAGGIAYEIDIHQNREYKNLYNEHKKLVSRVLSISDGQEIATLNQKIVEL |
| | KEQMKQITKINSIKRLEYKLRLAFGFIYTEYKNYEEFKNSFDTDIKNGRFT |
| | PKDEDGNKRAFDSRELEHLKGYYKATLQTQKPQTDEKMEEVSKRVDRL |
| | SLKSLIGDDTLLKFILLMFTFMPQELKGEFLGFIKKYYHDTKHIDQDTISD |
| | SDDTIEEGLSIGLKLKILDKNIRSLSILKHSLSFQTKYNKKDRSYYEDGNIH |
| | GKFFKKLGISHNQEEFNKSVYAPLFRYYSALYKLINDFEIYTLSLHIVGNE |
| | TLSDQVNKPQFLSGRYFNFRKLLTQSYNISNNSTHSVIFNAVINMRNDISH |
| | LSYEPLLDCPLNGKKSYKRKIRNQFRTINIKPLVESRKMIIDFITLQTDMQ |
| | KVLGCDAVNDFTMKIVQLRTRLKAYANKEQTIEKMITEAKTPNDFYNIY |
| | KVKGVEAINKYLLEVIGETQVEKEIREEIERGNIANS |
| SEQ ID NO: | MLKKPSNRYALPKVILSTVDHEKILEFKVKYEKLARLDRLVVERMHFDG |
| 115 | ESVVFDEVIANSGDLEIAYQDDHRKLLIQAAGKSYTITGKKVGGKKRKL |
| | EERISRAKIQLTLTDGQEDQHRRIRATVTEKALLEPKEDRDIYSKISDRKI |
| | KTSKEIYLVKRFLSYRSDLLFYYFFVDNFFKVGNNKQELWKIKFQNQPEL |
| | IEYFRFIINDRFKNAKNDKFDNYLKNDKAIQEDLEKIQKVFEKLRHALMH |
| | YDYGFFEKLFGGEDQGFDLDIAFLDNFVKKIDKLNIDTKKEFVDDEKIKIF |
| | GEDLNLADLYKLYASISINRVGFNRVVNEMIIKDGIEKSELKRAFEKKLD |
| | KTYALDIHSDPSYKKLYNEHKRLVTEVSTYTDGNKIKEGNQKIAKLKYE |
| | MKEITKKNALVRLECKMRLAFGLIYGRYDTHEAFKNGFDTDLKRGEFA |
| | QIGSEEAIGYFNTTFEKSKPKSKEEIKKIARQIDNLSLSTLIEDDPLMKFIVL |
| | MFLFVPRELKGEFLGFWRKYYHDIHSIDSDAKSDEMPDEVSLSLKLKILT |
| | RNIRRLNLFEYSLSEKIKYSPKNTQFYTDKSPYQKVYKRLKISHNKEEFD |
| | KTLLVPLFRYYSILFKLINDFEIYSLAKANPDASSLSELTKTKHGFRGHYN |
| | FTTLMMDAHKVSQGDSKKHFGIRGEIAHINTKDLIYDPLFRKSKMAQQR |
| | NDVIDFVLKYEKEIKAVLGYDAINDFRMKVVQLRTKLKVYSDKTQTIEK |
| | LLNEVEAPDDFYVLYKVKGVEAINKYLLEIVSVTQAEEEIERKIITGNKR |
| | YNT |
| SEQ ID NO: | MIKSYDDFTKLYSLQKTLRFELKPIGKTLEHIKKSEIIESDETLEEQYAIVK |
| 116 | NIIDRFHRKHIDEALSLVDFTKHLDTFKTIQELYLKRGKTDREKKELEELS |
| | ADLRKLIVSYLKGNVKQKTQHNLNPIKERFEILFGKELFTNEEFFTLAEN |
| | KEEKKAIQAFKGFTTYFKGFQENRKNMYSEEDKSTAIAYRIINENMPLFIE |
| | NIARFQKVLDVIEKTKLTELKQNLKTELKGHSVSDIFRIEYFNNVLTQEGI |
| | SRYNTIIGGKTTTEGVKIQGLNEIINLHNQQSKDVKLPILKPLHKQILSEEY |
| | STSFKIKAFENDNEVLKAIDTFWNEHIEKSIHPVTGKRFNILLKIENLCKKL |
| | EKYKDKEIEKLFIERKNLSTVSHQVYGQWNIIRDALRMHLEMNNKNIKE |
| | KDIDKYLDNDAFAWKEIKDSIKIYKEHVEDAKELDENGIVKYFSSMSINE |
| | EDDEKEYSISLIKNINEKYNNVKSILEEDRTGKSDLHQDKEKVAIIKEFLD |
| | SLKQLQWFLKLLYVTVPLDEKDYEFYNELEVYYEALLPLNSLYNKVRN |
| | YMTRKPYSVEKFKLNFYSPTLLDGWDKNKETANLSIILKKNGKYYLGIM |
| | NKENNTIFENFPKSKSNDYYEKMIYKLLPGPNKMLPKVFFSKKGLEYYK |
| | PSKEILRIYEKGEFKKDKSGNFKKESLHTLIDFYKEAIAKNEDWKIFKFKF |
| | KNTREYEDISQFYRDVEEQGYLIIFEKVDANYVDKLVEEGELYLFQIYNK |
| | DFSENKKSKGNPNLHTIYWESLFDNQNLKDVVYKLNGEAEVFYRKKSID |
| | YPEEIYNNGHHKEELNGKFNYPIIKDRRYTQDKFLFHVPITMNFISKEEKR |
| | VNQLACEYLSTTKEDVHIIGIDRGERHLLYLSLIDKEGNIKKQLSLNTIKN |
| | ENYDKEIDYRVKLDEKEKKRDEARKNWDVIENIKELKEGYMSQVIHIIA |
| | KMMAEEKAILIMEDLNIGFKRGRFKVEKQVYQKFEKMMIDKLNYLVFK |
| | NKEPLEPGGSLNAYQLTSKFDSFKKLGKQSGFIFYVPSAYTSKIDPTTGFY |
| | NFIRVDVPNLEKGKEFFSKFEKIIYNTKEDYFEFHCKYGKFVPEPKNKDN |
| | DRKTKESLTYYNAIKDTVWVVCSTHHERYKIVRNKAGYYESQPVDVTK |
| | NLRDIFSEANINYSDGKDIKPIIIESNNAKLLKSIAEQLKLILAMRYNNGKH |
| | DDDEKDYILSPVKNKQGKFFCSLDGDQSLPINADANGAYNIALKGLLLIE |
| | KIKKQQGKAKDLYISNLEWFMFMMSR |
| SEQ ID NO: | MIKSYDDFTKLYSLQKTLRFELKPIGKTLEHIKKSEIIESDETLEEQYAIVK |
| 117 | NIIDRFHRKHIDEALSLVDFTKHLDTFKTIQELYLKRGKTDREKKELEELS |
| | ADLRKLIVSYLKGNVKQKTQHNLNPIKERFEILFGKELFTNEEFFTLAEN |
| | KEEKKAIQAFKGFTTYFKGFQENRKNMYSEEDKSTAIAYRIINENMPLFIE |
| | NIARFQKVLDVIEKTKLTELKQNLKTELKGHSVSDIFRIEYFNNVLTQEGI |
| | SRYNTIIGGKTTTEGVKIQGLNEIINLHNQQSKDVKLPILKPLHKQILSEEY |
| | STSFKIKAFENDNEVLKAIDTFWNEHIEKSIHPVTGKRFNILLKIENLCKKL |
| | EKYKDKEIEKLFIERKNLSTVSHQVYGQWNIIRDALRMHLEMNNKNIKE |
| | KDIDKYLDNDAFAWKEIKDSIKIYKEHVEDAKELDENGIVKYFSSMSINE |
| | EDDEKEYSISLIKNINEKYNNVKSILEEDRTGKSDLHQDKEKVAIIKEFLD |
| | SLKQLQWFLKLLYVTVPLDEKDYEFYNELEVYYEALLPLNSLYNKVRN |
| | YMTRKPYSVEKFKLNFYSPTLLDGWDKNKETANLSIILKKNGKYYLGIM |
| | NKENNTIFENFPKSKSNDYYEKMIYKLLPGPNKMLPKVFFSKKGLEYYK |
| | PSKEILRIYEKGEFKKDKSGNFKKESLHTLIDFYKEAIAKNEDWKIFKFKF |
| | KNTREYEDISQFYRDVEEQGYLIIFEKVDANYVDKLVEEGELYLFQIYNK |
| | DFSENKKSKGNPNLHTIYWESLFDNQNLKDVVYKLNGEAEVFYRKKSID |
| | YPEEIYNNGHHKEELNGKFNYPIIKDRRYTQDKFLFHVPITMNFISKEEKR |
| | VNQLACEYLSTTKEDVHIIGIDRGERHLLYLSLIDKEGNIKKQLSLNTIKN |
| | ENYDKEIDYRVKLDEKEKKRDEARKNWDVIENIKELKEGYMSQVIHIIA |
| | KMMAEEKAILIMEDLNIGFKRGRFKVEKQVYQKFEKMMIDKLNYLVFK |
| | NKEPLEPGGSLNAYQLTSKFDSFKKLGKQSGFIFYVPSAYTSKIDPTTGFY |
| | NFIRVDVPNLEKGKEFFSKFEKIIYNTKEDYFEFHCKYGKFVPEPKNKDN |
| | DRKTKESLTYYNAIKDTVWVVCSTHHERYKIVRNKAGYYESQPVDVTK |
| | NLRDIFSEANINYSDGKDIKPIIIESNNAKLLKSIAEQLKLILAMRYNNGKH |
| | DDDEKDYILSPVKNKQGKFFCSLDGDQSLPINADANGAYNIALKGLLLIE |
| | KIKKQQGKAKDLYISNLEWFMFMMSR |
| SEQ ID NO: | MKSIFDNFTGLYSLSKTLRFELRPVGQTLENIKNGHFLESDKKMADDYQ |
| 118 | DVKKIIDNYHKFFIDDVLKGASFDWALLEKELTDFNKNKTDDSKVEAEQ |
| | KKLREQIAKTLAGDKRFKSLTASTPNDLFNKDKDFIGWLEQSSVKEIRKD |
| | ALDTFKKFSSYFKGFQENRKNVYSADDIPTAVPYRIVNDNFPKFLQNISIF |
| | KTIQEKCPQVIADVENELASYLGKEKLADIFTVQAFNKYLCQGGKENQR |
| | GIDFYNQVIGGIAEKEGGVNLRGINQFLNLYWQQHPDFAKENRRIKMVP |
| | LYKQILSDRSSLSFKIETIDTDEELKTAISEYADKLESKSNDEKKSVLDVC |
| | VELFDSIKEQNLQEIYVNRKDINNISRILTGDWSWLQSRMNLYADEVFTT |
| | KAEKTRWQKSVDGDEGENKSKGAYSLAELNRVLEYASENVAETDIRITD |
| | YFCHRNRFYYEKESGLFKQGEELVALSIKESCEDILSKRKAMNEAFANIS |
| | ESNSLRDNSEDIAKTKTYLDSVQDLLHRIKPLKVNGLGDPSFYAVFDSIY |
| | SALSEVISIYNKTRNYITRKAESPEKYKLNFDNPTLANGWDLNKEKDNTC |
| | VLLRKNGMYYLGIMNPKDKPKFAEKYDCGTESCYEKMIYKLLPGPNKM |
| | LPKVFFSTKGKKQYNPPENILHGYEQGKHKKGVAFDINFCHELIDWFKS |
| | AINQHEDWKKFGFKFSDTKSYKDISDFYREVTEQGYKLTFINIPESEISKM |
| | VSEGKLYLFQIYNMDFAPGANGMPNMHTLYWKNLFSEENLKDVVLKL |
| | NGEAELFYRPAGIKEPVVHAKGSYLVNRITKDGEPIPEKIHDEIYRNANG |
| | NLNNLSKEASEYKESHKVVIKQASHEIIKDRHYTEPKFLFHIPLTINFKAPS |
| | MKPTVCTAIQNENVRRFLKNNPDVNIIGLDRGERNLIYLSLINQKGEIIKQ |
| | FSFNDVEREQNGQTVKVNYHEKLDQREKERDAARKSWQAIGKIAELKE |
| | GYLSAVIHQLTKLMVEYNAIVVMEDLNFGFKRGRFHVEKQVYQKFEHM |
| | LIDKLNYLVFKDRGLNEPGGALNGYQLAGQFESFQKLGKQSGMLFYVP |
| | AAYTSKIDPKTGFVSMMNFKDLTNVHKKRDFFSKFEDIHFDEATCSFVFT |
| | FDYKNINGKAKEEMKQTKWAVYSREKRIVYFSKTKSSEDIMPTEKLRAL |
| | FESDGIEYKSGNNIHDSVMAVGADLKEGAKPSKEIADFWDGLLYNFKLI |
| | LQMRNSNAKTGEDYIISPVMASDGTFFDSRVEAKKGKDAKLPLDADAN |
| | GAYHIALKGLSLINKINLAGEDELKKFDMKISNEDWFNFAQEKKYAE |
| SEQ ID NO: | MTKQNKSVTQNTKRKNFGEFTNLYSLSKTLRFELKPVKATKTILEVERK |
| 119 | DRENFKKDRKIAKNYQKLKGILNELHQEFIQDVMREFSFQEKEIKEFEER |
| | YLEALNFKEKDNYKKRTQLKNAYEKVAKKLAGKIATAFGKYNQEKYG |
| | VKFTKKNLTGENVFDILEGKYKGDKKILGIIHTFKFKPTKEEKKQGKEAV |
| | NFSTYLGGFNQNRENFYKGEMKAGQFATRTIENLIQFLKNKKLFIDKYK |
| | DNYQKIGFSQEQVEIFNLNYFNNLFLQEGLDVYNGILGAKKGEKNTEND |
| | GLNQKINLFKQKEKTRCKANGEKFNKSDYPIFKELYKQIGSIKKDNDVY |
| | VEIKSDEELVNVLQSLPEKTANTLREVQKFYENFFDKIFNDEFDLDKIYLP |
| | KSVGTHFSHLAFSDWSKLAFVFNKRWRNEKVKIKEGEDVNVQSRSLADI |
| | KKRMEEILEMDGGVSFGKTYCQKVGLEKEARTIEDVWSGFWKIIQYHIN |
| | SQFIGGEKEVFDKEKKDDKTEKIQTIDDLQEEYLQATEMYRERMVESEE |
| | GLNDGEEKEIKTKLKNYLDRIKDIERIARYFDLRKHFDDIDEASKDGDFY |
| | FIYQELLQDISEAKINDHYNEIRNYLTKANVVDDKFKLNFNDGQTLSGW |
| | DLNKETEKFSLIFKRKVDGGVEYYLGIINKEKNKTIFDKKKHPEIFTENSE |
| | FEKMEYKLFPSPSKMLPKIAFTKNKEGERIKPVFLDENAGKEIAQIKKEFA |
| | LFQDAKKEDKNKWSDEFDRKKLNKLIDYYKLVLEKHPEKYMQTFNFVF |
| | KSSAKYKNLGEFNDDVARQNYVTKFVSVDKDYIDQKVESGELYLFKIH |
| | NKDWNLTKAGDTKKQSKKNLHTIYFEELFSEKNIAEPVFKLSGGAEVFF |
| | RDAIEKKKQKKKKDKKGKEILEKFRFTKNKILFHVPITINYGKPSINQGQF |
| | NQKINEFIADNSRSVNILGIDRGEKHLLYYSLVDNNGKIIKSGSLNEINGV |
| | DYHEKLDKAEKERQEARKSWQKINQIKNLKAGYISQVIKKIVDLAIENN |
| | AIIVLEDLNFGFKSFRQKIEKNVYQQFEKALIDKLGFVTDKEKLNHRQAP |
| | QLSAPFESFEKMGKQTGIVFYVLATNTSKVCPQCQWKKNIFPHYSTKKSI |
| | AENLQKQYKMKMYWRENENRFEFEYKGDGDKEFSSIFSNVDRVRYDK |
| | RANNNQGGYVIYQIDSTTKEKDGRNIKEKSITNLLKELLLEKFEIDNLEGE |
| | LLVKLSEKSPDVSKETIKDFFGLLNSILNIRNSMTDTEEDYIQCPACGFDT |
| | RKENKIGIKNGDDNGAYNIALRGRFLIERIKKAKKEDKKPNLTFSNNDYF |
| | QWVREFVK |
| SEQ ID NO: | MRTTTSLDAFTNRYALSKTLRFELKPIGNTQMMLEQNNVFAKDRAIREK |
| 120 | YEKTKPWIDLLHREFVAESLQNAQLGNLDDYYAALQNVQKITKDTNAE |
| | DKKRWKKGFEKQEKRLRKEVVALFDKAAHIWATQRYPQLKKKTKDFL |
| | FEEGVFEHVLFARYGSAPDTTVKIVTSNPETGEVIDEREESIFKGWKGFTG |
| | YFDKFFETRKNFYKDNGTATAIATRAINQNLRRFAENMQKLTDIKNNYP |
| | ELLAHTDFGDFDIAHAQSLDFYARTCLLQEGIDAYNKKFVGVLKSAINE |
| | YQQKNKGVRISYPKTLDNQILGERERRLFDVIEDDRELHDVFRAFVDDG |
| | TVFAAEMRQLAQAFSAQNGTYDYTQIYISKKGFETISRKYTHDTRAWHD |
| | ALADVFKAKAKKRIATTASGEKKFPAYIPVAYITQALTLVQESEDTECT |
| | WKERYASITENKTLEEGFFAIFADEFERLFVHMEATVQDTDYVVAEDKA |
| | KKLLSDGQITKNEQTTQIIKEYADALLRIYQMAKYFAVEKKSMWDDAV |
| | AIDDTFYETFKEIYGNTHSTIVASYNLLRNYLTKKPWEDVQKWKLNFEN |
| | PTLLDGWDKNKEAANFGVILRDGDKFYLGIMRKGHNNIFANQHHSNFE |
| | GQGLQKMVYKFFPDPKKMFPKVCFSAKGMEFFAPSEEIVRIYKNAEFKS |
| | GDTFNVESMQKLIDFYKNALQKYDGWKIYDFKHLKDTAQYTSNIGEFY |
| | DDVAKGGYQLGWQNISKEYVEEKNANGELYLFQIKNKDWNDGATGRK |
| | NLHTLYFEYLFSEKNAAADFVFRLNGGAEVFYRPAAIESKTERRGNREV |
| | AAKKRYTQDKVFLHVPITLNRTAGDVKTSAFNDAVNRFLAGNPDINIMG |
| | IDRGEKHLAYYSIIDQNGNRIVSGSFNTIGSKDYHALLTERQGAREEARK |
| | NWQRVEQIKDLKKGYISLVVREIADLAIKHNAIIVLENLNMRFKQIRGGIE |
| | KSVYQQLEKALIEKLNFLVNKGEVDATKAGHLLRAYQLAAPFETFEKM |
| | GNQTGIIFYTTASYTSQVDPVTGWRPHVYLKYRNAQKTKEDILRIFDDIV |
| | FNDEKQRFEFAYRHNGVSWTVCSSVERHRWNRSNNAGKGGYDVFPVE |
| | GEGSITQRLQEACAQRGIDTTRNILAQIDELDESASATVSFLRDLCFYFRLI |
| | CQIRNTDDGADDINAQDFLMSPVEPFFDTRNAQEQYPQNGDENGAYNIA |
| | RKGIIILQKITAWGRSQDTQRRYPDTFVSQDEWDTFLTQHTT |
| SEQ ID NO: | MLKEKQFKTFGDFTNLYELSKTLRFELRPTPETKDLLDKNKIIQTDKKIAE |
| 121 | NYQEIKKYFDKLHKKFIKEALSNTQIDFSDFCKLWEQNSKDSGKIKDLSR |
| | KLRKSIKQAFDKKGADWHKRYLEKGIKLKKKNLDILFEERVLDILKEEF |
| | KDDVDVKLFESFKGFSTYFTNFHESRKNFYKDDGTASAIATRIIDENLKR |
| | FCDNIKVKKHSKKLISELNEREAKIFEADFYNRCLLQQGIDDYNQVIGDIN |
| | KKINNLRQNKIENTPTLKILYKQILGDVRRQETEQDAFIEIKNNEEVFDFL |
| | QDFIKHSDENNKYFKNLFYKFIEGKHSLDKIFLAKRFVNTISGKWFASWE |
| | VFGAELIKKFGNKKDLPDFIPFAAVKDVLQNCNIPANELFKEKIKNDEDK |
| | NIYDIFINLWKEEFDSNLKKVEESKKEVENMIAEDKVYSNKKEKRKNDN |
| | GEEIEIEIQKEKIKNYADAAMNIFRMMKYFLLEKNGKTVEGMGEDNNFY |
| | NELNIVFKGGEIDGKVYEGVKTYLYYNEFRNYLTKKPFNEEKTKLNFDC |
| | GQILSGWDKNKESEKLGVILRKDNKYYLAIINKKHNKIFDVKKNSYAYI |
| | VGDNFYEKMEYKLFPDAKRMIPKIAFAKNNKEKFGWTDEIQKIKNEYAE |
| | FQEGKKNDKNLWKDKFNKNKMEKLITYYQNCLEKGGYKDIYNFRWKS |
| | PDKYSGIGEFNDEIDRQSYCLKFVKVDFNYVFEKVKSGELYLFQIYNKDF |
| | SDKADRAQKENIHTEYFKLLFDQRNLDNVVLKLSGGAEIFYRPKTEGLP |
| | KKKDNKGNEVVRHRRYADDKYFLHLPIQLNFGRGNLSGGEFNSKINQY |
| | LSEQREIKIIGIDRGEKHLAYYSVINQDGKIEEIESLNTVNGIDYRKKLDEL |
| | EKKREQERKSWQSISKIKDLKKGYISHVIKKICDLAIEHNAIIVFEDLSGGF |
| | KNSRKKIEKQIYQNLELALATKLNYLTFKDKNFGESGHYLNAYQLAPKI |
| | DNYQDIKMQTGIVFYTPAGYTSSTCPQCGFRKTLKFDYTATISKAEDLIR |
| | GSKLNIVFEKEKNRFKINYLFNPIEKKKKKIKENELFADAGAKNEFTIYSD |
| | VKRIKWHNTGTKRLEEAEGERLLENKNSRGRDKEYDINKCLTRLFRENK |
| | IDVNGDIIGQITKIKSLKLYQDLFYYLFLATLIRNNVSGSDIDYIQCPSCHF |
| | HSDGGFQKQKFNGDANGAYNIARKGILILKKIKQFAAQDKDMKNFGWK |
| | HLTVDINEWDKFTQK |
| SEQ ID NO: | MNKNFSNFTELYTLSKTLRFELKPVAQTKENIKKGKFLESDKKKADDYK |
| 122 | DVKKIIDNYHKFFIDDVLKNASFDWTVLEKEMSDFNKSKADDSKVEAEQ |
| | KKLRDQIAKKLTSDKRFKALTASTPSDLFNKDKDFIDWFTQNSTKDINKE |
| | ALETFKRFSSYFKGFQENRKNVYSAEPIPTAVPYRLVNDNFPKFLQNIALF |
| | KIIQEKCPQVISDVEKELASYLGKEKLADIFTVQAFNKYLCQGGKENQRG |
| | IDFYNNILGGIAEKEGGINLRGINQFLNLYWQQHQDFAKQNKRIKMIPLF |
| | KQILSDRSSLSFKIESINTDQELLTSITEYADKLETKSNDEKKSVLDICSDL |
| | FASIKAQNLQEIYVNRKDINSISRILTGDWSWLQSRMNVYADEVFTTKAE |
| | KTRWQKSIDGDEGENKSKGVFSLAELNSVLEYSSENVSETDVRITDFFDH |
| | RNRFYYEKESGLFKQGDELVALSIRESCEDILAKRKAMDEAFANVSENN |
| | SLRDNSEDVAKIKIYLDCVQELLHRIKPLKVNGLGDPAFYAVFDTVYNSL |
| | SEVISLYNKTRNYITKKAANPEKYKLNFDNPTLADGWDLNKEQANTSVL |
| | MRKDGMYYLGIMNPKDKPKFAEKYECGNEACYEKMIYKQFDATKQIPK |
| | CSTQVKEVKKHFQSGATDSIILNDKSKFKLDLVITKEIWFLNNHVWNGE |
| | KFVPKRESNETRPKKFQIGYYKQTGDLGGYKEALNIWISFCKTFLQSYISS |
| | SIYDYDFKESSNYDSLDEFYNYLNATCYKLSFINIPEATISQMVSEGKLYL |
| | FQIYNKDFAPGASGMPNMHTLYWKNLFSEENLKNVVLKLNGEAELFYR |
| | PAGIKEPVIHAKGSYLVNRITKDGEPIPEKIHDEIYRNANGKLESLSKEAT |
| | EYKASHKVVIKEAKHDIIKDRHYTEPKFLFHVPLTINFKASGNSYINENVR |
| | RFLKNNPDVNVIGLDRGERHLIYLSLINQKGEIIKQFTFNEVERNKNGQVI |
| | KVNYHEKLDQREKVRGAARKSWQAIGKIAELKEGYLSAVIHQLTKLMV |
| | EYNAIVVMEDLNFGFKRGRFHVEKQVYQKFEHMLIDKLNYLVFKDRGL |
| | TEAGGVLNGYQLAGQFESFQKLGKQSGMLFYVPAGYTSKIDPKTGFAS |
| | MSNFKDLTNVHKKRAFFSKFDDIHFDDATGSFVFTFDYKNFDGKAKEE |
| | MKRTKWSVYSKDKRIVYLSKTKSYEDVQPTEKIKASLESVGIEYMSGNN |
| | LIDSIMVIGAELKDGAKPSKEIADFWDRLLYNFKLILQMRNSNAKTGEDY |
| | IISPVMADDGTFFDSREEFKKGENAKMPVDADANGAYHIALKGLSLLKR |
| | FDAASENELKKFDMKISNVDWFKFAQEKSYAE |
| SEQ ID NO: | MKAKKSFYNQKRKFGKRGYRLHDERIAYSGGIGSMRSIKYELKDSYGIA |
| 123 | GLRNRIADATISDNKWLYGNINLNDYLEWRSSKTDKQIEDGDRESSLLGF |
| | WLEALRLGFVFSKQSHAPNDFNETALQDLFETLDDDLKHVLDRKKWCD |
| | FIKIGTPKTNDQGRLKKQIKNLLKGNKREEIEKTLNESDDELKEKINRIAD |
| | VFAKNKSDKYTIFKLDKPNTEKYPRINDVQVAFFCHPDFEEITERDRTKT |
| | LDLIINRFNKRYEITENKKDDKTSNRMALYSLNQGYIPRVLNDLFLFVKD |
| | NEDDFSQFLSDLENFFSFSNEQIKIIKERLKKLKKYAEPIPGKPQLADKWD |
| | DYASDFGGKLESWYSNRIEKLKKIPESVSDLRNNLEKIRNVLKKQNNAS |
| | KILELSQKIIEYIRDYGVSFEKPEIIKFSWINKTKDGQKKVFYVAKMADRE |
| | FIEKLDLWMADLRSQLNEYNQDNKVSFKKKGKKIEELGVLDFALNKAK |
| | KNKSTKNENGWQQKLSESIQSAPLFFGEGNRVRNEEVYNLKDLLFSEIK |
| | NVENILMSSEAEDLKNIKIEYKEDGAKKGNYVLNVLARFYARFNEDGYG |
| | GWNKVKTVLENIAREAGTDFSKYGNNNNRNAGRFYLNGRERQVFTLIK |
| | FEKSITVEKILELVKLPSLLDEAYRDLVNENKNHKLRDVIQLSKTIMALV |
| | LSHSDKEKQIGGNYIHSKLSGYNALISKRDFISRYSVQTTNGTQCKLAIGK |
| | GKSKKGNEIDRYFYAFQFFKNDDSKINLKVIKNNSHKNIDENDNENKINA |
| | LQVYSSNYQIQFLDWFFEKHQGKKTSLEVGGSFTIAEKSLTIDWSGSNPR |
| | VGFKRSDTEEKRVFVSQPFTLIPDDEDKERRKERMIKTKNRFIGIDIGEYG |
| | LAWSLIEVDNGDKNNRGIRQLESGFITDNQQQVLKKNVKSWRQNQIRQT |
| | FTSPDTKIARLRESLIGSYKNQLESLMVAKKANLSFEYEVSGFEVGGKRV |
| | AKIYDSIKRGSVRKKDNNSQNDQSWGKKGINEWSFETTAAGTSQFCTHC |
| | KRWSSLAIVDIEEYELKDYNDNLFKVKINDGEVRLLGKKGWRSGEKIKG |
| | KELFGPVKDAMRPNVDGLGMKIVKRKYLKLDLRDWVSRYGNMAIFICP |
| | YVDCHHISHADKQAAFNIAV |
| SEQ ID NO: | MKLTRRRISGNSVDQKITAAFYRDMSQGLLYYDSEDNDCTDKVIESMDF |
| 124 | ERSWRGRILKNGEDDKNPFYMFVKGLVGSNDKIVCEPIDVDSDPDNLDI |
| | LINKNLTGFGRNLKAPDSNDTLENLIRKIQAGIPEEEVLPELKKIKEMIQK |
| | DIVNRKEQLLKSIKNNRIPFSLEGSKLVPSTKKMKWLFKLIDVPNKTFNE |
| | KMLEKYWEIYDYDKLKANITNRLDKTDKKARSISRAVSEELREYHKNLR |
| | TNYNRFVSGDRPAAGLDNGGSAKYNPDKEEFLLFLKEVEQYFKKYFPV |
| | KSKHSNKSKDKSLVDKYKNYCSYKVVKKEVNRSIINQLVAGLIQQGKLL |
| | YYFYYNDTWQEDFLNSYGLSYIQVEEAFKKSVMTSLSWGINRLTSFFIDD |
| | SNTVKFDDITTKKAKEAIESNYFNKLRTCSRMQDHFKEKLAFFYPVYVK |
| | DKKDRPDDDIENLIVLVKNAIESVSYLRNRTFHFKESSLLELLKELDDKN |
| | SGQNKIDYSVAAEFIKRDIENLYDVFREQIRSLGIAEYYKADMISDCFKTC |
| | GLEFALYSPKNSLMPAFKNVYKRGANLNKAYIRDKGPKETGDQGQNSY |
| | KALEEYRELTWYIEVKNNDQSYNAYKNLLQLIYYHAFLPEVRENEALIT |
| | DFINRTKEWNRKETEERLNTKNNKKHKNFDENDDITVNTYRYESIPDYQ |
| | GESLDDYLKVLQRKQMARAKEVNEKEEGNNNYIQFIRDVVVWAFGAY |
| | LENKLKNYKNELQPPLSKENIGLNDTLKELFPEEKVKSPFNIKCRFSISTFI |
| | DNKGKSTDNTSAEAVKTDGKEDEKDKKNIKRKDLLCFYLFLRLLDENEI |
| | CKLQHQFIKYRCSLKERRFPGNRTKLEKETELLAELEELMELVRFTMPSIP |
| | EISAKAESGYDTMIKKYFKDFIEKKVFKNPKTSNLYYHSDSKTPVTRKY |
| | MALLMRSAPLHLYKDIFKGYYLITKKECLEYIKLSNIIKDYQNSLNELHE |
| | QLERIKLKSEKQNGKDSLYLDKKDFYKVKEYVENLEQVARYKHLQHKI |
| | NFESLYRIFRIHVDIAARMVGYTQDWERDMHFLFKALVYNGVLEERRFE |
| | AIFNNNDDNNDGRIVKKIQNNLNNKNRELVSMLCWNKKLNKNEFGAII |
| | WKRNPIAHLNHFTQTEQNSKSSLESLINSLRILLAYDRKRQNAVTKTIND |
| | LLLNDYHIRIKWEGRVDEGQIYFNIKEKEDIENEPIIHLKHLHKKDCYIYK |
| | NSYMFDKQKEWICNGIKEEVYDKSILKCIGNLFKFDYEDKNKSSANPKH |
| | T |
| SEQ ID NO: | MLRRDKEVKKLYNVFNQIQVGTKPKKWNNDEKLSPEENERRAQQKNIK |
| 125 | MKNYKWREACSKYVESSQRIINDVIFYSYRKAKNKLRYMRKNEDILKK |
| | MQEAEKLSKFSGGKLEDFVAYTLRKSLVVSKYDTQEFDSLAAMVVFLE |
| | CIGKNNISDHEREIVCKLLELIRKDFSKLDPNVKGSQGANIVRSVRNQNMI |
| | VQPQGDRFLFPQVYAKENETVINKNVEKEGLNEFLLNYANLDDEKRAE |
| | SLRKLRRILDVYFSAPNHYEKDMDITLSDNIEKEKFNVWEKHECGKKET |
| | GLFVDIPDVLMEAEAENIKLDAVVEKRERKVLNDRVRKQNIICYRYTRA |
| | VVEKYNSNEPLFFENNAINQYWIHHIENAVERILKNCKAGKLFKLRKGY |
| | LAEKVWKDAINLISIKYIALGKAVYNFALDDIWKDKKNKELGIVDERIRN |
| | GITSFDYEMIKAHENLQRELAVDIAFSVNNLARAVCDMSNLGNKESDFL |
| | LWKRNDIADKLKNKDDMASVSAVLQFFGGKSSWDINIFKDAYKGKKKY |
| | NYEVRFIDDLRKAIYCARNENFHFKTALVNDEKWNTELFGKIFERETEFC |
| | LNVEKDRFYSNNLYMFYQVSELRNMLDHLYSRSVSRAAQVPSYNSVIV |
| | RTAFPEYITNVLGYQKPSYDADTLGKWYSACYYLLKEIYYNSFLQSDRA |
| | LQLFEKSVKTLSWDDKKQQRAVDNFKDHFSDIKSACTSLAQVCQIYMTE |
| | YNQQNNQIKKVRSSNDSIFDQPVYQHYKVLLKKAIANAFADYLKNNKD |
| | LFGFIGKPFKANEIREIDKEQFLPDWTSRKYEALCIEVSGSQELQKWYIVG |
| | KFLNARSLNLMVGSMRSYIQYVTDIKRRAASIGNELHVSVHDVEKVEK |
| | WVQVIEVCSLLASRTSNQFEDYFNDKDDYARYLKSYVDFSNVDMPSEY |
| | SALVDFSNEEQSDLYVDPKNPKVNRNIVHSKLFAADHILRDIVEPVSKDN |
| | IEEFYSQKAEIAYCKIKGKEITAEEQKAVLKYQKLKNRVELRDIVEYGEII |
| | NELLGQLINWSFMRERDLLYFQLGFHYDCLRNDSKKPEGYKNIKVDENS |
| | IKDAILYQIIGMYVNGVTVYAPEKDGDKLKEQCVKGGVGVKVSAFHRY |
| | SKYLGLNEKTLYNAGLEIFEVVAEHEDIINLRNGIDHFKYYLGDYRSMLS |
| | IYSEVFDRFFTYDIKYQKNVLNLLQNILLRHNVIVEPILESGFKTIGEQTKP |
| | GAKLSIRSIKSDTFQYKVKGGTLITDAKDERYLETIRKILYYAENEEDNLK |
| | KSVVVTNADKYEKNKESDDQNKQKEKKNKDNKGKKNEETKSDAEKNN |
| | NERLSYNPFANLNFKLSN |

Other Exemplary protein sequences are described in the following applications: PCT/US2021/033271; PCT/US2021/035031, and PCT/US2022/028865, all of which are herein incorporated by reference in their entirety.

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF (SEQ ID NO: 128). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF (SEQ ID NO: 128). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

### Cas14 Proteins

In some instances, the TypeV CRISPR/Cas protein comprises a Cas14 protein. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between β-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas 14 proteins may comprise a RuvC domain or a partial RuvC domain. The RuvC domain may be defined by a single, contiguous sequence, or a set of partial RuvC domains that are not contiguous with respect to the primary amino acid sequence of the Cas 14 protein. In some instances, a partial RuvC domain does not have any substrate binding activity or catalytic activity on its own. A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas 14 protein, but form a RuvC domain once the protein is produced and folds. A Cas14 protein may comprise a linker loop connecting a carboxy terminal domain of the Cas 14 protein with the amino terminal domain of the Cas 14 protein, and wherein the carboxy terminal domain comprises one or more RuvC domains and the amino terminal domain comprises a recognition domain.

Cas 14 proteins may comprise a zinc finger domain. In some instances, a carboxy terminal domain of a Cas 14 protein comprises a zinc finger domain. In some instances, an amino terminal domain of a Cas14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

Cas 14 proteins may be relatively small compared to many other Cas proteins, making them suitable for nucleic acid detection or gene editing. For instance, a Cas14 protein may be less likely to adsorb to a surface or another biological species due to its small size. The smaller nature of these proteins also allows for them to be more easily packaged as a reagent in a system or assay, and delivered with higher efficiency as compared to other larger Cas proteins. In some cases, a Cas14 protein is 400 to 800 amino acid residues long, 400 to 600 amino acid residues long, 440 to 580 amino acid residues long, 460 to 560 amino acid residues long, 460 to 540 amino acid residues long, 460 to 500 amino acid residues long, 400 to 500 amino acid residues long, or 500 to 600 amino acid residues long. In some cases, a Cas14 protein is less than about 550 amino acid residues long. In some cases, a Cas14 protein is less than about 500 amino acid residues long.

In some instances, a Cas 14 protein may function as an endonuclease that catalyzes cleavage at a specific position within a target nucleic acid. In some instances, a Cas14 protein is capable of catalyzing non-sequence-specific cleavage of a single stranded nucleic acid. In some cases, a Cas 14 protein is activated to perform trans cleavage activity after binding of a guide nucleic acid with a target nucleic acid. This trans cleavage activity is also referred to as “collateral” or “transcollateral” cleavage. Trans cleavage activity may be non-specific cleavage of nearby single-stranded nucleic acid by the activated programmable nuclease, such as trans cleavage of detector nucleic acids with a detection moiety.

### Engineered Programmable Nuclease Probes

Disclosed herein are non-naturally occurring compositions and systems comprising at least one of an engineered Cas protein and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid may comprise a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally-occurring sequence. In some instances, the guide nucleic acid may comprise two naturally occurring sequences arranged in an order or proximity that is not observed in nature. In some instances, compositions and systems comprise a ribonucleotide complex comprising a CRISPR/Cas effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein comprise a multi-vector system encoding a Cas protein and a guide nucleic acid described herein, wherein the guide nucleic acid and the Cas protein are encoded by the same or different vectors. In some embodiments, the engineered guide and the engineered Cas protein are encoded by different vectors of the system.

### Programmable Nuclease Fusion Proteins

Described herein are various embodiments of programmable nuclease fusion proteins. In some embodiments, a programmable nuclease may be fused with a heterologous polypeptide to form a programmable nuclease fusion protein. In some instances, “fusion partner” can refer to a polypeptide that is fused to another polypeptide. In some embodiments, the heterologous polypeptide may be an enzyme, wherein the enzyme is configured to react an enzyme substrate to generate a detectable signal. In some embodiments, the heterologous polypeptide may provide a tag (i.e., the heterologous polypeptide is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag (SEQ ID NO: 129); a hemagglutinin (HA) tag; a FLAG tag; a Myc tag; and the like).

“Heterologous,” as used herein, can refer to a nucleotide or polypeptide sequence that is not found in the native nucleic acid or protein, respectively. In some cases, a heterologous polypeptide may comprise a sequence different from a sequence of a given protein. For example, a sequence of an HRP enzyme may be considered heterologous to a sequence of a programmable nuclease when the sequence of the HRP enzyme is different from the sequence of the programmable nuclease. In some embodiments, a programmable nuclease (e.g., a Cas12 protein) can be fused to an active domain from a non-programmable nuclease protein (e.g., an HRP), and the sequence of the active domain may be considered to be a heterologous polypeptide (i.e., it is heterologous to the programmable nuclease).

### Thermostable Programmable Nuclease

Described herein are various embodiments of thermostable programmable nucleases. In some embodiments, a programmable nuclease is referred to as an effector protein. An effector protein may be thermostable. In some instances, known effector proteins (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of an effector protein in a trans cleavage assay at 40° C. may be at least 50% of that at 37° C. In some instances, the trans cleavage activity of an effector protein in a trans cleavage assay at 40° C. may be at least 55% of that at 37° C. In some instances, the trans cleavage activity of an effector protein in a trans cleavage assay at 40° C. may be at least 60% of that at 37° C. In some instances, the trans cleavage activity of an effector protein in a trans cleavage assay at 40° C. may be at least 65% of that at 37° C. In some instances, the trans cleavage activity of an effector protein in a trans cleavage assay at 40° C. may be at least 70% of that at 37° C. In some instances, the trans cleavage activity of an effector protein in a trans cleavage assay at 40° C. may be at least 75% of that at 37° C. In some instances, the trans cleavage activity of an effector protein in a trans cleavage assay at 40° C. may be at least 80% of that at 37° C. In some instances, the trans cleavage activity of an effector protein in a trans cleavage assay at 40° C. may be at least 85% of that at 37° C. In some instances, the trans cleavage activity of an effector protein in a trans cleavage assay at 40° C. may be at least 90% of that at 37° C. In some instances, the trans cleavage activity of an effector protein in a trans cleavage assay at 40° C. may be at least 95% of that at 37° C. In some instances, the trans cleavage activity of an effector protein in a trans cleavage assay at 40° C. may be at least 100% of that at 37° C. In some instances, the trans cleavage activity of an effector protein in a trans cleavage assay at 40° C. may be at least 1-fold of that at 37° C. In some instances, the trans cleavage activity of an effector protein in a trans cleavage assay at 40° C. may be at least 2-fold of that at 37° C. In some instances, the trans cleavage activity of an effector protein in a trans cleavage assay at 40° C. may be at least 3-fold of that at 37° C. In some instances, the trans cleavage activity of an effector protein in a trans cleavage assay at 40° C. may be at least 4-fold of that at 37° C. In some instances, the trans cleavage activity of an effector protein in a trans cleavage assay at 40° C. may be at least 5-fold of that at 37° C. In some instances, the trans cleavage activity of an effector protein in a trans cleavage assay at 40° C. may be at least 6-fold of that at 37° C. In some instances, the trans cleavage activity of an effector protein in a trans cleavage assay at 40° C. may be at least 7-fold of that at 37° C. In some instances, the trans cleavage activity of an effector protein in a trans cleavage assay at 40° C. may be at least 8-fold of that at 37° C. In some instances, the trans cleavage activity of an effector protein in a trans cleavage assay at 40° C. may be at least 9-fold of that at 37° C. In some instances, the trans cleavage activity of an effector protein in a trans cleavage assay at 40° C. may be at least 10-fold of that at 37° C. In some instances, the trans cleavage activity of an effector protein in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 45° C. may be at least 50% of that at 37° C. In some instances, the trans cleavage activity of an effector protein in a trans cleavage assay at 45° C. may be at least 55% of that at 37° C. In some instances, the trans cleavage activity of an effector protein in a trans cleavage assay at 45° C. may be at least 60% of that at 37° C. In some instances, the trans cleavage activity of an effector protein in a trans cleavage assay at 45° C. may be at least 65% of that at 37° C. In some instances, the trans cleavage activity of an effector protein in a trans cleavage assay at 45° C. may be at least 70% of that at 37° C. In some instances, the trans cleavage activity of an effector protein in a trans cleavage assay at 45° C. may be at least 75% of that at 37° C. In some instances, the trans cleavage activity of an effector protein in a trans cleavage assay at 45° C. may be at least 80% of that at 37° C. In some instances, the trans cleavage activity of an effector protein in a trans cleavage assay at 45° C. may be at least 85% of that at 37° C. In some instances, the trans cleavage activity of an effector protein in a trans cleavage assay at 45° C. may be at least 90% of that at 37° C. In some instances, the trans cleavage activity of an effector protein in a trans cleavage assay at 45° C. may be at least 95% of that at 37° C. In some instances, the trans cleavage activity of an effector protein in a trans cleavage assay at 45° C. may be at least 100% of that at 37° C. In some instances, the trans cleavage activity of an effector protein in a trans cleavage assay at 45° C. may be at least 1-fold of that at 37° C. In some instances, the trans cleavage activity of an effector protein in a trans cleavage assay at 45° C. may be at least 2-fold of that at 37° C. In some instances, the trans cleavage activity of an effector protein in a trans cleavage assay at 45° C. may be at least 3-fold of that at 37° C. In some instances, the trans cleavage activity of an effector protein in a trans cleavage assay at 45° C. may be at least 4-fold of that at 37° C. In some instances, the trans cleavage activity of an effector protein in a trans cleavage assay at 45° C. may be at least 5-fold of that at 37° C. In some instances, the trans cleavage activity of an effector protein in a trans cleavage assay at 45° C. may be at least 6-fold of that at 37° C. In some instances, the trans cleavage activity of an effector protein in a trans cleavage assay at 45° C. may be at least 7-fold of that at 37° C. In some instances, the trans cleavage activity of an effector protein in a trans cleavage assay at 45° C. may be at least 8-fold of that at 37° C. In some instances, the trans cleavage activity of an effector protein in a trans cleavage assay at 45° C. may be at least 9-fold of that at 37° C. In some instances, the trans cleavage activity of an effector protein in a trans cleavage assay at 45° C. may be at least 10-fold of that at 37° C. In some instances, the trans cleavage activity of an effector protein in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 50° C. may be at least 50% of that at 37° C. In some instances, the trans cleavage activity of an effector protein in a trans cleavage assay at 50° C. may be at least 55% of that at 37° C. In some instances, the trans cleavage activity of an effector protein in a trans cleavage assay at 50° C. may be at least 60% of that at 37° C. In some instances, the trans cleavage activity of an effector protein in a trans cleavage assay at 50° C. may be at least 65% of that at 37° C. In some instances, the trans cleavage activity of an effector protein in a trans cleavage assay at 50° C. may be at least 70% of that at 37° C. In some instances, the trans cleavage activity of an effector protein in a trans cleavage assay at 50° C. may be at least 75% of that at 37° C. In some instances, the trans cleavage activity of an effector protein in a trans cleavage assay at 50° C. may be at least 80% of that at 37° C. In some instances, the trans cleavage activity of an effector protein in a trans cleavage assay at 50° C. may be at least 85% of that at 37° C. In some instances, the trans cleavage activity of an effector protein in a trans cleavage assay at 50° C. may be at least 90% of that at 37° C. In some instances, the trans cleavage activity of an effector protein in a trans cleavage assay at 50° C. may be at least 95% of that at 37° C. In some instances, the trans cleavage activity of an effector protein in a trans cleavage assay at 50° C. may be at least 100% of that at 37° C. In some instances, the trans cleavage activity of an effector protein in a trans cleavage assay at 50° C. may be at least 1-fold of that at 37° C. In some instances, the trans cleavage activity of an effector protein in a trans cleavage assay at 50° C. may be at least 2-fold of that at 37° C. In some instances, the trans cleavage activity of an effector protein in a trans cleavage assay at 50° C. may be at least 3-fold of that at 37° C. In some instances, the trans cleavage activity of an effector protein in a trans cleavage assay at 50° C. may be at least 4-fold of that at 37° C. In some instances, the trans cleavage activity of an effector protein in a trans cleavage assay at 50° C. may be at least 5-fold of that at 37° C. In some instances, the trans cleavage activity of an effector protein in a trans cleavage assay at 50° C. may be at least 6-fold of that at 37° C. In some instances, the trans cleavage activity of an effector protein in a trans cleavage assay at 50° C. may be at least 7-fold of that at 37° C. In some instances, the trans cleavage activity of an effector protein in a trans cleavage assay at 50° C. may be at least 8-fold of that at 37° C. In some instances, the trans cleavage activity of an effector protein in a trans cleavage assay at 50° C. may be at least 9-fold of that at 37° C. In some instances, the trans cleavage activity of an effector protein in a trans cleavage assay at 50° C. may be at least 10-fold of that at 37° C. In some instances, the trans cleavage activity of an effector protein in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 55° C. may be at least 50% of that at 37° C. In some instances, the trans cleavage activity of an effector protein in a trans cleavage assay at 55° C. may be at least 55% of that at 37° C. In some instances, the trans cleavage activity of an effector protein in a trans cleavage assay at 55° C. may be at least 60% of that at 37° C. In some instances, the trans cleavage activity of an effector protein in a trans cleavage assay at 55° C. may be at least 65% of that at 37° C. In some instances, the trans cleavage activity of an effector protein in a trans cleavage assay at 55° C. may be at least 70% of that at 37° C. In some instances, the trans cleavage activity of an effector protein in a trans cleavage assay at 55° C. may be at least 75% of that at 37° C. In some instances, the trans cleavage activity of an effector protein in a trans cleavage assay at 55° C. may be at least 80% of that at 37° C. In some instances, the trans cleavage activity of an effector protein in a trans cleavage assay at 55° C. may be at least 85% of that at 37° C. In some instances, the trans cleavage activity of an effector protein in a trans cleavage assay at 55° C. may be at least 90% of that at 37° C. In some instances, the trans cleavage activity of an effector protein in a trans cleavage assay at 55° C. may be at least 95% of that at 37° C. In some instances, the trans cleavage activity of an effector protein in a trans cleavage assay at 55° C. may be at least 100% of that at 37° C. In some instances, the trans cleavage activity of an effector protein in a trans cleavage assay at 55° C. may be at least 1-fold of that at 37° C. In some instances, the trans cleavage activity of an effector protein in a trans cleavage assay at 55° C. may be at least 2-fold of that at 37° C. In some instances, the trans cleavage activity of an effector protein in a trans cleavage assay at 55° C. may be at least 3-fold of that at 37° C. In some instances, the trans cleavage activity of an effector protein in a trans cleavage assay at 55° C. may be at least 4-fold of that at 37° C. In some instances, the trans cleavage activity of an effector protein in a trans cleavage assay at 55° C. may be at least 5-fold of that at 37° C. In some instances, the trans cleavage activity of an effector protein in a trans cleavage assay at 55° C. may be at least 6-fold of that at 37° C. In some instances, the trans cleavage activity of an effector protein in a trans cleavage assay at 55° C. may be at least 7-fold of that at 37° C. In some instances, the trans cleavage activity of an effector protein in a trans cleavage assay at 55° C. may be at least 8-fold of that at 37° C. In some instances, the trans cleavage activity of an effector protein in a trans cleavage assay at 55° C. may be at least 9-fold of that at 37° C. In some instances, the trans cleavage activity of an effector protein in a trans cleavage assay at 55° C. may be at least 10-fold of that at 37° C. In some instances, the trans cleavage activity of an effector protein in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 60° C. may be at least 50% of that at 37° C. In some instances, the trans cleavage activity of an effector protein in a trans cleavage assay at 60° C. may be at least 55% of that at 37° C. In some instances, the trans cleavage activity of an effector protein in a trans cleavage assay at 60° C. may be at least 60% of that at 37° C. In some instances, the trans cleavage activity of an effector protein in a trans cleavage assay at 60° C. may be at least 65% of that at 37° C. In some instances, the trans cleavage activity of an effector protein in a trans cleavage assay at 60° C. may be at least 70% of that at 37° C. In some instances, the trans cleavage activity of an effector protein in a trans cleavage assay at 60° C. may be at least 75% of that at 37° C. In some instances, the trans cleavage activity of an effector protein in a trans cleavage assay at 60° C. may be at least 80% of that at 37° C. In some instances, the trans cleavage activity of an effector protein in a trans cleavage assay at 60° C. may be at least 85% of that at 37° C. In some instances, the trans cleavage activity of an effector protein in a trans cleavage assay at 60° C. may be at least 90% of that at 37° C. In some instances, the trans cleavage activity of an effector protein in a trans cleavage assay at 60° C. may be at least 95% of that at 37° C. In some instances, the trans cleavage activity of an effector protein in a trans cleavage assay at 60° C. may be at least 100% of that at 37° C. In some instances, the trans cleavage activity of an effector protein in a trans cleavage assay at 60° C. may be at least 1-fold of that at 37° C. In some instances, the trans cleavage activity of an effector protein in a trans cleavage assay at 60° C. may be at least 2-fold of that at 37° C. In some instances, the trans cleavage activity of an effector protein in a trans cleavage assay at 60° C. may be at least 3-fold of that at 37° C. In some instances, the trans cleavage activity of an effector protein in a trans cleavage assay at 60° C. may be at least 4-fold of that at 37° C. In some instances, the trans cleavage activity of an effector protein in a trans cleavage assay at 60° C. may be at least 5-fold of that at 37° C. In some instances, the trans cleavage activity of an effector protein in a trans cleavage assay at 60° C. may be at least 6-fold of that at 37° C. In some instances, the trans cleavage activity of an effector protein in a trans cleavage assay at 60° C. may be at least 7-fold of that at 37° C. In some instances, the trans cleavage activity of an effector protein in a trans cleavage assay at 60° C. may be at least 8-fold of that at 37° C. In some instances, the trans cleavage activity of an effector protein in a trans cleavage assay at 60° C. may be at least 9-fold of that at 37° C. In some instances, the trans cleavage activity of an effector protein in a trans cleavage assay at 60° C. may be at least 10-fold of that at 37° C. In some instances, the trans cleavage activity of an effector protein in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 65° C. may be at least 50% of that at 37° C. In some instances, the trans cleavage activity of an effector protein in a trans cleavage assay at 65° C. may be at least 55% of that at 37° C. In some instances, the trans cleavage activity of an effector protein in a trans cleavage assay at 65° C. may be at least 60% of that at 37° C. In some instances, the trans cleavage activity of an effector protein in a trans cleavage assay at 65° C. may be at least 65% of that at 37° C. In some instances, the trans cleavage activity of an effector protein in a trans cleavage assay at 65° C. may be at least 70% of that at 37° C. In some instances, the trans cleavage activity of an effector protein in a trans cleavage assay at 65° C. may be at least 75% of that at 37° C. In some instances, the trans cleavage activity of an effector protein in a trans cleavage assay at 65° C. may be at least 80% of that at 37° C. In some instances, the trans cleavage activity of an effector protein in a trans cleavage assay at 65° C. may be at least 85% of that at 37° C. In some instances, the trans cleavage activity of an effector protein in a trans cleavage assay at 65° C. may be at least 90% of that at 37° C. In some instances, the trans cleavage activity of an effector protein in a trans cleavage assay at 65° C. may be at least 95% of that at 37° C. In some instances, the trans cleavage activity of an effector protein in a trans cleavage assay at 65° C. may be at least 100% of that at 37° C. In some instances, the trans cleavage activity of an effector protein in a trans cleavage assay at 65° C. may be at least 1-fold of that at 37° C. In some instances, the trans cleavage activity of an effector protein in a trans cleavage assay at 65° C. may be at least 2-fold of that at 37° C. In some instances, the trans cleavage activity of an effector protein in a trans cleavage assay at 65° C. may be at least 3-fold of that at 37° C. In some instances, the trans cleavage activity of an effector protein in a trans cleavage assay at 65° C. may be at least 4-fold of that at 37° C. In some instances, the trans cleavage activity of an effector protein in a trans cleavage assay at 65° C. may be at least 5-fold of that at 37° C. In some instances, the trans cleavage activity of an effector protein in a trans cleavage assay at 65° C. may be at least 6-fold of that at 37° C. In some instances, the trans cleavage activity of an effector protein in a trans cleavage assay at 65° C. may be at least 7-fold of that at 37° C. In some instances, the trans cleavage activity of an effector protein in a trans cleavage assay at 65° C. may be at least 8-fold of that at 37° C. In some instances, the trans cleavage activity of an effector protein in a trans cleavage assay at 65° C. may be at least 9-fold of that at 37° C. In some instances, the trans cleavage activity of an effector protein in a trans cleavage assay at 65° C. may be at least 10-fold of that at 37° C. In some instances, the trans cleavage activity of an effector protein in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of an effector protein in a trans cleavage assay at 70° C., 75° C. 80° C., or more may be at least 50, at least 60%, at least 65%, at least 70%, at least 75%, at least 80%, at least 85%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity may be measured against a negative control in a trans cleavage assay. In some instances, the trans cleavage activity of an effector protein against a nucleic acid in a trans cleavage assay at 37° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 37° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 65° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 70° C., 75° C., 80° C., or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 70° C., 75° C., 80° C., or more may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid.

The reporters described herein can be RNA reporters. The RNA reporters can comprise at least one ribonucleic acid and a detectable moiety. In some embodiments, a programmable nuclease probe or a CRISPR probe comprising a Cas enzyme can recognize and detect ssDNA and, further, can specifically trans-cleave RNA reporters. The detection of the target nucleic acid in the sample can indicate the presence of the disease (or disease-causing agent) in the sample and can provide information for taking action to reduce the transmission of the disease to individuals in the disease-affected environment or near the disease-carrying individual.

Cleavage of a reporter (e.g., a protein-nucleic acid) can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

In an aspect, the present disclosure provides a method for target detection. The method can comprise sample collection. The method can further comprise sample preparation. The method can further comprise detection of one or more target nucleic acids in the collected and prepared sample.

In another aspect, the present disclosure provides a detection device for target detection. The detection device can be configured for multiplexed target detection. The detection device can be used to collect one or more samples, prepare or process the one or more samples for detection, and optionally divide the one or more samples into a plurality of droplets, aliquots, or subsamples for amplification of one or more target sequences or target nucleic acids. The target sequences may comprise, for example, a biological sequence. The biological sequence can comprise a nucleic acid sequence or an amino acid sequence. In some embodiments, the target sequences are associated with an organism of interest, a disease of interest, a disease state of interest, a phenotype of interest, a genotype of interest, or a gene of interest.

The detection device can be configured to amplify target nucleic acids contained within the plurality of droplets, aliquots, or subsamples. The detection device can be configured to amplify the target sequences or target nucleic acids contained within the plurality of droplets by individually processing each of the plurality of droplets (e.g., by using a thermocycling process or any other suitable amplification process as described in greater detail below). In some cases, the plurality of droplets can undergo separate thermocycling processes. In some cases, the thermocycling processes can occur simultaneously. In other cases, the thermocycling processes can occur at different times for each droplet.

The detection device can be further configured to remix the droplets, aliquots, or subsamples after the target nucleic acids in each of the droplets undergo amplification. The detection device can be configured to provide the remixed sample comprising the droplets, aliquots, or subsamples to a detection chamber of the device. The detection chamber can be configured to direct the remixed droplets, aliquots, or subsamples to a plurality of programmable nuclease probes. The detection chamber can be configured to direct the remixed droplets, aliquots, or subsamples along one or more fluid flow paths such that the remixed droplets, aliquots, or subsamples are positioned adjacent to and/or in contact with the one or more programmable nuclease probes. In some cases, the detection chamber can be configured to recirculate or recycle the remixed droplets, aliquots, or subsamples such that the remixed droplets, aliquots, or subsamples are repeatedly placed in contact with one or more programmable nuclease probes over a predetermined period of time.

The detection device can comprise one or more sensors. The one or more sensors of the detection device can be configured to detect one or more signals that are generated after one or more programmable nucleases of the one or more programmable nuclease probes become activated due to a binding of a guide nucleic acid of the programmable nuclease probes with a target nucleic acid present in the sample. As described elsewhere herein, the activated programmable nuclease can cleave the target nucleic acid, which can result in a trans cleavage activity. Trans cleavage activity can be a non-specific cleavage of nearby single-stranded nucleic acids by the activated programmable nuclease, such as trans cleavage of target nucleic acids with a detection moiety. Once the target nucleic acids are cleaved by the activated programmable nucleases, the detection moiety can be released or separated from the reporter, thereby generating one or more detectable signals. The one or more sensors of the detection device can be configured to register and/or process the one or more detectable signals to confirm a presence and/or an absence of a particular target (e.g., a target nucleic acid).

The one or more programmable nuclease probes of the detection device can be configured for multiplexed detection. In some cases, each programmable nuclease probe can be configured to detect a particular target. In other cases, each programmable nuclease probe can be configured to detect a plurality of targets. In some cases, a first programmable nuclease probe can be configured to detect a first target or a first set of targets, and a second programmable nuclease probe can be configured to detect a second target or a second set of targets. In other cases, a first programmable nuclease probe can be configured to detect a first set of targets, and a second programmable nuclease probe can be configured to detect a second set of targets. The programmable nuclease probes of the present disclosure can be used to detect a plurality of different target sequences or target nucleic acids. In any of the embodiments described herein, the sample provided to the detection device can comprise a plurality of target sequences or target nucleic acids. In any of the embodiments described herein, the sample provided to the detection device can comprise multiple classes of target sequences or target nucleic acids. Each class of target sequences or class of target nucleic acids can comprise a plurality of target sequences or target nucleic acids associated with a particular organism, disease state, phenotype, or genotype present within the sample. In some cases, each programmable nuclease probe can be used to detect a particular class of target sequences or a particular class of target nucleic acids associated with a particular organism, disease state, phenotype, or genotype present within the sample. In some cases, two or more programmable nuclease probes can be used to detect different classes of target sequences or different classes of target nucleic acids. In such cases, the two or more programmable nuclease probes can comprise different sets or classes of guide nucleic acids complexed to the programmable nucleases of the probes.

In any of the embodiments described herein, the detection device can comprise a single integrated system that is configured to perform sample collection, sample processing, droplet generation, droplet processing (e.g., amplification of target nucleic acids in droplets), droplet remixing, and/or circulation of the remixed droplets within a detection chamber so that at least a portion of the remixed droplets is placed in contact with one or more programmable nuclease probes coupled to the detection chamber. The detection devices of the present disclosure can be disposable devices configured to perform one or more rapid single reaction or multi-reaction tests to detect a presence and/or an absence of one or more target sequences or target nucleic acids.

The systems and methods of the present disclosure can be used to detect one or more target sequences or nucleic acids in one or more samples. The one or more samples can comprise one or more target sequences or nucleic acids for detection of an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, any combination thereof. A sample can be an aspirate of a bodily fluid from an animal (e.g., human, animals, livestock, pet, etc.) or plant. A tissue sample can be from any tissue that can be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water, or soil. A sample from an environment can be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system. In some cases, the sample is contained in no more than about 200 nanoliters (nL). In some cases, the sample is contained in about 200 nL. In some cases, the sample is contained in a volume that is greater than about 200 nL and less than about 20 microliters (μL). In some cases, the sample is contained in no more than 20 μl. In some cases, the sample is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl. In some cases, the sample is contained in from 1 μL to 500 μL, from 10 μL to 500 μL, from 50 μL to 500 μL, from 100 μL to 500 μL, from 200 μL to 500 μL, from 300 μL to 500 μL, from 400 μL to 500 μL, from 1 μL to 200 μL, from 10 μL to 200 μL, from 50 μL to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL. Sometimes, the sample is contained in more than 500 μl.

In some instances, the sample is taken from a single-cell eukaryotic organism; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample may comprise nucleic acids from a cell lysate from a eukaryotic cell, a mammalian cell, a human cell, a prokaryotic cell, or a plant cell. In some cases, the sample may comprise nucleic acids expressed from a cell.

The sample used for disease testing can comprise at least one target sequence that can bind to a guide nucleic acid of the reagents described herein. In some cases, the target sequence is a portion of a nucleic acid. A nucleic acid can be from a genomic locus, a transcribed mRNA, or a reverse transcribed cDNA. A nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A nucleic acid can be from 10 to 90, from 20 to 80, from 30 to 70, or from 40 to 60 nucleotides in length. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-CoV-2 (i.e., a virus that causes COVID-19), SARS, MERS, influenza, Adenovirus, Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus/Enterovirus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus) and respiratory bacteria (e.g., Bordetella parapertussis, Bordetella pertussis, Chlamydia pneumoniae, Mycoplasma pneumoniae). Other viruses include human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, Chlamydia pneumoniae, Chlamydia psittaci, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemmorhagic viral diseases (e.g., Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Bacillus anthracis, Bortadella pertussis, Burkholderia cepacia, Corynebacterium diphtheriae, Coxiella burnetii, Streptococcus agalactiae, methicillin-resistant Staphylococcus aureus, Legionella longbeachae, Legionella pneumophila, Leptospira interrogans, Moraxella catarrhalis, Streptococcus pyogenes, Escherichia coli, Neisseria gonorrhoeae, Neisseria meningitidis, Neisseria elongate, Neisseria gonorrhoeae, Parechovirus, Pneumococcus, Pneumocystis jirovecii, Cryptococcus neoformans, Histoplasma capsulatum, Haemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid may comprise a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Staphylococcus epidermidis, Legionella pneumophila, Streptococcus pyogenes, Streptococcus salivarius, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the Coronavirus HKU1 sequence is a target of an assay. In some embodiments, the Coronavirus NL63 sequence is a target of an assay. In some embodiments, the Coronavirus 229E sequence is a target of an assay. In some embodiments, the Coronavirus OC43 sequence is a target of an assay. In some embodiments, the SARS-CoV-1 sequence is a target of an assay. In some embodiments, the MERS sequence is a target of an assay. In some embodiments, the SARS-CoV-2 sequence is a target of an assay. In some embodiments, the Respiratory Syncytial Virus A sequence is a target of an assay. In some embodiments, the Respiratory Syncytial Virus B sequence is a target of an assay. In some embodiments, the Influenza A sequence is a target of an assay. In some embodiments, the Influenza B sequence is a target of an assay. In some embodiments, the Human Metapneumovirus sequence is a target of an assay. In some embodiments, the Human Rhinovirus sequence is a target of an assay. In some embodiments, the Human Enterovirus sequence is a target of an assay. In some embodiments, the Parainfluenza Virus 1 sequence is a target of an assay. In some embodiments, the Parainfluenza Virus 2 sequence is a target of an assay. In some embodiments, the Parainfluenza Virus 3 sequence is a target of an assay. In some embodiments, the Parainfluenza Virus 4 sequence is a target of an assay. In some embodiments, the Alphacoronavirus genus sequence is a target of an assay. In some embodiments, the Betacoronavirus genus sequence is a target of an assay. In some embodiments, the Sarbecovirus subgenus sequence is a target of an assay. In some embodiments, the SARS-related virus species sequence is a target of an assay. In some embodiments, the Gammacoronavirus Genus sequence is a target of an assay. In some embodiments, the Deltacoronavirus Genus sequence is a target of an assay. In some embodiments, the Influenza B-Victoria V1 sequence is a target of an assay. In some embodiments, the Influenza B-Yamagata Y1 sequence is a target of an assay. In some embodiments, the Influenza A H1 sequence is a target of an assay. In some embodiments, the Influenza A H2 sequence is a target of an assay. In some embodiments, the Influenza A H3 sequence is a target of an assay. In some embodiments, the Influenza A H4 sequence is a target of an assay. In some embodiments, the Influenza A H5 sequence is a target of an assay. In some embodiments, the Influenza A H6 sequence is a target of an assay. In some embodiments, the Influenza A H7 sequence is a target of an assay. In some embodiments, the Influenza A H8 sequence is a target of an assay. In some embodiments, the Influenza A H9 sequence is a target of an assay. In some embodiments, the Influenza A H10 sequence is a target of an assay. In some embodiments, the Influenza A H11 sequence is a target of an assay. In some embodiments, the Influenza A H12 sequence is a target of an assay. In some embodiments, the Influenza A H13 sequence is a target of an assay. In some embodiments, the Influenza A H14 sequence is a target of an assay. In some embodiments, the Influenza A H15 sequence is a target of an assay. In some embodiments, the Influenza A H16 sequence is a target of an assay. In some embodiments, the Influenza A N1 sequence is a target of an assay. In some embodiments, the Influenza A N2 sequence is a target of an assay. In some embodiments, the Influenza A N3 sequence is a target of an assay. In some embodiments, the Influenza A N4 sequence is a target of an assay. In some embodiments, the Influenza A N5 sequence is a target of an assay. In some embodiments, the Influenza A N6 sequence is a target of an assay. In some embodiments, the Influenza A N7 sequence is a target of an assay. In some embodiments, the Influenza A N8 sequence is a target of an assay. In some embodiments, the Influenza A N9 sequence is a target of an assay. In some embodiments, the Influenza A N10 sequence is a target of an assay. In some embodiments, the Influenza A N11 sequence is a target of an assay. In some embodiments, the Influenza A/H1-2009 sequence is a target of an assay. In some embodiments, the Human endogenous control 18S rRNA sequence is a target of an assay. In some embodiments, the Human endogenous control GAPDH sequence is a target of an assay. In some embodiments, the Human endogenous control HPRT1 sequence is a target of an assay. In some embodiments, the Human endogenous control GUSB sequence is a target of an assay. In some embodiments, the Human endogenous control RNASe P sequence is a target of an assay. In some embodiments, the Influenza A oseltamivir resistance sequence is a target of an assay. In some embodiments, the Human Bocavirus sequence is a target of an assay. In some embodiments, the SARS-CoV-2 85Δ sequence is a target of an assay. In some embodiments, the SARS-CoV-2 T1001I sequence is a target of an assay. In some embodiments, the SARS-CoV-2 3675-36774 sequence is a target of an assay. In some embodiments, the SARS-CoV-2 P4715L sequence is a target of an assay. In some embodiments, the SARS-CoV-2 S5360L sequence is a target of an assay. In some embodiments, the SARS-CoV-2 69-70Δ sequence is a target of an assay. In some embodiments, the SARS-CoV-2 Tyr144fs sequence is a target of an assay. In some embodiments, the SARS-CoV-2 242-244Δ sequence is a target of an assay. In some embodiments, the SARS-CoV-2 Y453F sequence is a target of an assay. In some embodiments, the SARS-CoV-2 S477N sequence is a target of an assay. In some embodiments, the SARS-CoV-2 E848K sequence is a target of an assay. In some embodiments, the SARS-CoV-2 N501Y sequence is a target of an assay. In some embodiments, the SARS-CoV-2 D614G sequence is a target of an assay. In some embodiments, the SARS-CoV-2 P681R sequence is a target of an assay. In some embodiments, the SARS-CoV-2 P681H sequence is a target of an assay. In some embodiments, the SARS-CoV-2 L21F sequence is a target of an assay. In some embodiments, the SARS-CoV-2 Q27Stop sequence is a target of an assay. In some embodiments, the SARS-CoV-2 M1fs sequence is a target of an assay. In some embodiments, the SARS-CoV-2 R203fs sequence is a target of an assay. In some embodiments, the Human adenovirus-pan assay sequence is a target of an assay. In some embodiments, the Bordetella parapertussis sequence is a target of an assay. In some embodiments, the Bordetella pertussis sequence is a target of an assay. In some embodiments, the Chlamydophila pneumoniae sequence is a target of an assay. In some embodiments, the Mycoplasma pneumoniae sequence is a target of an assay. In some embodiments, the Legionella pneumophila sequence is a target of an assay. In some embodiments, the Bordetella bronchoseptica sequence is a target of an assay. In some embodiments, the Bordetella holmesii sequence is a target of an assay. In some embodiments, the Human adenovirus Type A sequence is a target of an assay. In some embodiments, the Human adenovirus Type B sequence is a target of an assay. In some embodiments, the Human adenovirus Type C sequence is a target of an assay. In some embodiments, the Human adenovirus Type D sequence is a target of an assay. In some embodiments, the Human adenovirus Type E sequence is a target of an assay. In some embodiments, the Human adenovirus Type F sequence is a target of an assay. In some embodiments, the Human adenovirus Type G sequence is a target of an assay. In some embodiments, the MERS-CoV sequence is a target of an assay. In some embodiments, the human metapneumovirus sequence is a target of an assay. In some embodiments, the human parainfluenza 1 sequence is a target of an assay. In some embodiments, the human parainfluenza 2 sequence is a target of an assay. In some embodiments, the human parainfluenza 4 sequence is a target of an assay. In some embodiments, the hCoV-OC43 sequence is a target of an assay. In some embodiments, the human parainfluenza 3 sequence is a target of an assay. In some embodiments, the RSV-A sequence is a target of an assay. In some embodiments, the RSV-B sequence is a target of an assay. In some embodiments, the hCoV-229E sequence is a target of an assay. In some embodiments, the hCoV-HKU1 sequence is a target of an assay. In some embodiments, the hCoV-NL63 sequence is a target of an assay. In some embodiments, the Gammacoronavirus sequence is a target of an assay. In some embodiments, the Deltacoronavirus sequence is a target of an assay. In some embodiments, the Alphacoronavirus sequence is a target of an assay. In some embodiments, the Rhinovirus C sequence is a target of an assay. In some embodiments, the Betacoronavirus sequence is a target of an assay. In some embodiments, the Influenza A sequence is a target of an assay. In some embodiments, the Influenza B sequence is a target of an assay. In some embodiments, the SARS-CoV-2 sequence is a target of an assay. In some embodiments, the SARS-CoV-1 sequence is a target of an assay. In some embodiments, the Sarbecovirus subgenus sequence is a target of an assay. In some embodiments, the SARS-related viruses sequence is a target of an assay. In some embodiments, the MS2 sequence is a target of an assay.

In some embodiments, the assay is directed to one or more target sequences. In some embodiments, a target sequence is a portion of an antimicrobial resistance (AMR) gene, such as CTX-M-1, CTX-M-2, CTX-M-25, CTX-M-8, CTX-M-9, or IMP. In some embodiments, a target sequence is a Mycobacterium tuberculosis sequence, such as a portion of IS1081 or IS6110. In some embodiments, a target sequence is an orthopox virus sequence. In some embodiments, a target sequence is a pseudorabies virus sequence. In some embodiments, a target sequence is a Staphylococcus aureus sequence, such as a portion of gyrA or gyrB, or a portion of a S. aureus thermonuclease. In some embodiments, a target sequence is a Stenotrophomonas maltophilia sequence, such as a sequence of S. maltophilia alpha, S. maltophilia beta, or S. maltophilia gamma. In some embodiments, a target sequence is a Bordetalla sp. sequence, such as a sequence of Bordetella bronchoseptica, Bordetella holmesii, Bordetella parapertussis, or Bordetella pertussis. In some embodiments, a target sequence is a Chlamydophila pneumoniae sequence. In some embodiments, a target sequence is a Human adenovirus sequence, such as a sequence of human adenovirus Type A, Type B, Type C, Type D, Type E, Type F, or Type G. In some embodiments, a target sequence is a human bocavirus sequence. In some embodiments, a target sequence is a Legionella pneumophila sequence. In some embodiments, a target sequence is a Mycoplasma pneumoniae sequence. In some embodiments, a target sequence is an Acinetobacter spp. (e.g., A. pitii, A. baumannii, or A. nosocomialis) sequence, such as a portion of gyrB or a 16S-23S ribosomal RNA intergenic spacer sequence. In some embodiments, a target sequence is a Proteus spp. (e.g. P. mirabilis, P. vulgaris, P. penneri, or P. hauseri) sequence, such as a portion of rpoD or 16S. In some embodiments, a target sequence is an Enterobacter spp. (e.g. E. nimipressuralis, E. cloacae, E. asburiae, E. hormaechei, E. kobei, E. ludwigii, or E. mori) sequence, such as a portion of dnaJ, purG, or 16S. In some embodiments, a target sequence is a Bacillus anthracis sequence, such as a portion of pagA or capB. In some embodiments, a target sequence is a Brucella spp. sequence, such as a portion of 23S, bcsp31, or omp2a. In some embodiments, a target sequence is a Coxiella burnetiid sequence, such as a portion of com1 or IS110. In some embodiments, a target sequence is a Francisella tularensis sequence, such as a portion of 16S. In some embodiments, a target sequence is a Rickettsia spp. sequence, such as a portion of 16S, 23S, or 782-17K genus common antigen. In some embodiments, a target sequence is a Yersinia pestis sequence, such as a portion of pMT1, pCD1, or pPCP1. In some embodiments, a target sequence is a A. calcoaceticus sequence, such as a portion of gyrB. In some embodiments, a target sequence is a Francisella tularensis sequence, such as a portion of tul4 or fopA. In some embodiments, a target sequence is a Nocardia spp. sequence, such as a portion of 16S, hsp65, gyrB, secA1, or sodA. In some embodiments, a target sequence is a Cryptococcus spp. sequence, such as a portion of 18S, URA5, ITS, 28S, or CTX1. In some embodiments, a target sequence is an Actinomyces spp. sequence, such as a portion of 16S. In some embodiments, a target sequence is a Streptococcus spp. sequence, such as a portion of 16S, tuf, sodA, or rpoB. In some embodiments, a target sequence is an rRNA sequence, such as a portion of 28S rRNA or 18S rRNA. In some embodiments, a target sequence is a coronavirus sequence, such as a sequence of an alphacoronavirus, betacoronavirus, deltacoronavirus, or gammacoronavirus. In some embodiments, a target sequence is a human coronavirus (hCoV) sequence, such as a sequence of hCoV-229E, hCoV-HKU1, hCoV-NL63, hCoV-OC43. In some embodiments, a target sequence is a MERS-CoV sequence. In some embodiments, the sequence is a mammarenavirus sequence, such as a sequence of a Argentinian mammarenavirus (Junin arenavirus), Lassa mammarenavirus, Lujo mammarenavirus (e.g., an L segment or S segment thereof), or Machupo mammarenavirus. In some embodiments, a target sequence is a human metapneumovirus sequence. In some embodiments, a target sequence is a human parainfluenza sequence, such as a sequence of human parainfluenza 1, human parainfluenza 2, human parainfluenza 3, or human parainfluenza 4. In some embodiments, a target sequence is an influenza A virus sequence, such as a sequence of influenza A H1, H2, H3, H4, H5, H6, H7, H8, H9, H10, H11, H12, H13, H14, H15, H16, N1, N2, N3, N4, N5, N6, N7, N8, or N9. In some embodiments, a target sequence is an influenza B sequence, such as a sequence of influenza B-Victoria V1 or influenza B-Yamagata Y1. In some embodiments, a target sequence is a bacteriophage MS2 sequence. In some embodiments, a target sequence is a rhinovirus C sequence. In some embodiments, a target sequence is a respiratory syncytial virus (RSV) sequence, such as a sequence of RSV-A or RSV-B. In some embodiments, a target sequence is a Sarbecovirus sequence. In some embodiments, a target sequence is a severe acute respiratory syndrome coronavirus (SARS-CoV) sequence, such as a sequence of SARS-CoV-1 or SARS-CoV-2. In some embodiments, a target sequence is a portion of a SARS-CoV-2 S gene, such as a sequence comprising 144/145 wild-type (WT), deletion (del) 144/145 (alpha variant), 156/157 WT, del 156/157 (delta variant), 241/243 WT, del241/243 (beta variant), 69/70 WT, del69/70 (alpha variant), A570 WT, A570D (alpha variant), A701 WT, A701V (beta variant), D1118 WT, D1118H (alpha variant), D215 WT, D215G (beta variant), D614 WT, D614G (beta variant), D80 WT, D80A (beta variant), E484 WT, E484K (gamma variant), P681 WT, P681H (alpha variant), P681R (delta variant), S982 WT, S982A (alpha variant), T19 WT, T19R (delta variant), T716 WT, T716F (gamma variant). In some embodiments, a target sequence is a SARS-related virus sequence. In some embodiments, a target sequence is a portion of a gene selected from 16S, 18S, 23S, 28S, ACTB, ATP5ME, ATP5MF, ATP5MG, ATP5PB, BCSP31, CAPB, CHMP2A, Clorf43, COM1, CTX1, DNAJ, EMC7, FOPA, GPI, GAPDH, GUSB, GYRB, HRPT1, HSP65, NDUFB3, NDUFB4, NDUFB8, OMP2A, PAGA, PRDX1, PSMB2, PSMB4, PURG, RAB7A, REEP5, RNaseP, RPL13, RPL19, RPL27A, RPL30, RPL31, RPL32, RPL37A, RPOB, RPOD, RPS10, RPS27, RPS29, RPS6, SECA1, SNRPD3, SODA, TUF, TUL4, URA5, VCP, VPS29, and YWHAG.

In some embodiments, the one or more targets may be at a concentration of 1 copy/reaction, at least about 2 copies/reaction, at least about 3 copies/reaction, at least about 4 copies/reaction, at least about 5 copies/reaction, at least about 6 copies/reaction, at least about 7 copies/reaction, at least about 8 copies/reaction, at least about 9 copies/reaction, at least about 10 copies/reaction, at least about 20 copies/reaction, at least about 30 copies/reaction, at least about 40 copies/reaction, at least about 50 copies/reaction, at least about 60 copies/reaction, at least about 70 copies/reaction, at least about 80 copies/reaction, at least about 90 copies/reaction, at least about 100 copies/reaction, at least about 200 copies/reaction, at least about 300 copies/reaction, at least about 400 copies/reaction, at least about 500 copies/reaction, at least about 600 copies/reaction, at least about 700 copies/reaction, at least about 800 copies/reaction, at least about 900 copies/reaction, at least about 1000 copies/reaction, at least about 2000 copies/reaction, at least about 3000 copies/reaction, at least about 4000 copies/reaction, at least about 5000 copies/reaction, at least about 6000 copies/reaction, at least about 7000 copies/reaction, at least about 8000 copies/reaction, at least about 9000 copies/reaction, at least about 10000 copies/reaction, at least about 20000 copies/reaction, at least about 30000 copies/reaction, at least about 40000 copies/reaction, at least about 50000 copies/reaction, at least about 60000 copies/reaction, at least about 70000 copies/reaction, at least about 80000 copies/reaction, at least about 90000 copies/reaction, or at least about 100000 copies/reaction.

The sample used for cancer testing or cancer risk testing can comprise at least one target sequence or target nucleic acid segment that can bind to a guide nucleic acid of the reagents described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1 C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51 C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for genetic disorder testing can comprise at least one target sequence or target nucleic acid segment that can bind to a guide nucleic acid of the reagents described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1 C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNTI, PPT1, PROP1, PRPS1, PSAP, PTS, PUS1, PYGM, RAB23, RAG2, RAPSN, RARS2, RDH12, RMRP, RPE65, RPGRIPIL, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1 C, USH2A, VPS13A, VPS13B, VPS45, VRK1, VSX2, WNT10A, XPA, XPC, and ZFYVE26.

The sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

The sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

The sample used for ancestral testing can comprise at least one target nucleic acid segment that can bind to a guide nucleic acid of the reagents described herein. The target nucleic acid segment, in some cases, is a portion of a nucleic acid from a gene associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method may comprise obtaining a serum sample from a subject; and identifying a disease status of the subject. Often, the disease status is prostate disease status. In any of the embodiments described herein, the device can be configured for asymptomatic, pre-symptomatic, and/or symptomatic diagnostic applications, irrespective of immunity. In any of the embodiments described herein, the device can be configured to perform one or more serological assays on a sample (e.g., a sample comprising blood).

In some embodiments, the sample can be used to identify a mutation in a target nucleic acid of a plant or of a bacteria, virus, or microbe associated with a plant or soil. The devices and methods of the present disclosure can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid can be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the systems and methods disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250 to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the systems and methods disclosed herein. Some methods described herein can be implemented to detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the methods of the present disclosure can be implemented to detect target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

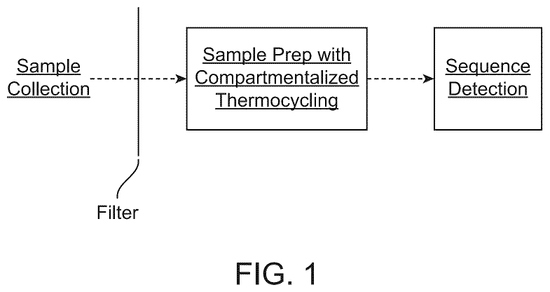

FIG. 1 illustrates an exemplary method for programmable nuclease-based detection. The method can comprise collecting a sample. The sample can comprise any type of sample as described herein. The method can comprise preparing the sample. Sample preparation can comprise one or more sample preparation steps. The one or more sample preparation steps can be performed in any suitable order. The one or more sample preparation steps can comprise physical filtration of non-target materials using a macro filter. The one or more sample preparation steps can comprise nucleic acid purification. The one or more sample preparation steps can comprise lysis. The one or more sample preparation steps can comprise heat inactivation. The one or more sample preparation steps can comprise enzyme (e.g., PK) inactivation. The one or more sample preparation steps can comprise adding one or more enzymes or reagents to prepare the sample for target detection.

The method can comprise generating one or more droplets, aliquots, or subsamples from the sample. The one or more droplets, aliquots, or subsamples can correspond to a volumetric portion of the sample. The sample can be divided into 1, 2, 3, 4, 5, 6, 7, 8, 9, 10, 20, 30, 40, 50, 60, 70, 80, 90, 100, 200, 300, 400, 500, 600, 700, 800, 900, 1000, or more droplets, aliquots, or subsamples. In some embodiments, the sample is not divided into subsamples.

The method can comprise amplifying one or more targets within each droplet, aliquot, or subsample. Amplification of the one or more targets within each droplet can be performed in parallel and/or simultaneously for each droplet. Dividing the sample into a plurality of droplets can enhance a speed and/or an efficiency of the amplification process (e.g., a thermocycling process) since the droplets comprise a smaller volume of material than the bulk sample introduced. Amplifying the one or more targets within each individual droplet can also permit effective amplification of various target nucleic acids that cannot be amplified as efficiently in a bulk sample containing the various target nucleic acids if the bulk sample were to undergo a singular amplification process. In some embodiments, amplification is performed on the bulk sample without first dividing the sample into subsamples.

The method can further comprise using a CRISPR-based or programmable nuclease-based detection module to detect one or more targets (e.g., target sequences or target nucleic acids) in the sample. In some cases, the sample can be divided into a plurality of droplets, aliquots, or subsamples to facilitate sample preparation and to enhance the detection capabilities of the devices of the present disclosure. In some cases, the sample is not divided into subsamples.

In some embodiments, the sample can be provided manually to the detection device of the present disclosure. For example, a swab sample can be dipped into a solution and the sample/solution can be pipetted into the device. In other embodiments, the sample can be provided via an automated syringe. The automated syringe can be configured to control a flow rate at which the sample is provided to the detection device. The automated syringe can be configured to control a volume of the sample that is provided to the detection device over a predetermined period.

In some embodiments, the sample can be provided directly to the detection device of the present disclosure. For example, a swab sample can be inserted into a sample chamber on the detection device.

The sample can be prepared before one or more targets are detected within the sample. The sample preparation steps described herein can process a crude sample to generate a pure or purer sample. Sample preparation can one or more physical or chemical processes, including, for example, nucleic acid purification, lysis, binding, washing, and/or eluting. In certain instances, sample preparation can comprise the following steps, in any order, including sample collection, nucleic acid purification, heat inactivation, enzyme inactivation, and/or base/acid lysis.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). Exemplary proteases include serine proteases (e.g., Proteinase K, Savinase®, trypsin, Protamex®, etc.), metalloproteinases (e.g., MMP-3, etc.), cysteine proteases (e.g, cathepsin B, papin, etc.), threonine proteases, aspartic proteases (e.g., renin, pepsin, cathepsin D, etc.), glutamic proteases, asparagine peptide lyases, or the like. In some cases, a solution of reagents can be used to lyse the cells in the sample and release the nucleic acids so that they are accessible to the programmable nuclease. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate·2H20, 0.5% (w/v) sodium lauryl sarcosinate, and 0.1 M β-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned previously. In some cases, depending on the type of sample, nanoscale barbs, nanowires, acoustic generators, integrated lasers, integrated heaters, and/or microcapillary probes can be used to perform lysis.

In certain instances, heat inactivation can be performed on the sample. In some embodiments, a processed/lysed sample can undergo heat inactivation to inactivate, in the lysed sample, the proteins used during lysing (e.g, a PK enzyme or a lysing reagent) and/or other residual proteins in the sample (e.g., RNases, DNases, viral proteins, etc.). In some cases, a heating element integrated into the detection device can be used for heat-inactivation. The heating element can be powered by a battery or another source of thermal or electric energy that is integrated with the detection device.

In certain instances, enzyme inactivation can be performed on the sample. In some embodiments, a processed/lysed sample can undergo enzyme inactivation to inhibit or inactivate, in the lysed sample, the proteins used during lysing (e.g., a PK enzyme or a lysing reagent) and/or other residual proteins in the sample (e.g., RNases, DNases, etc.). In some cases, a solution of reagents can be used to inactivate one or more enzymes present in the sample. Enzyme inactivation can occur before, during, or after lysis, when lysis is performed. For example, an RNase inhibitor may be included as a lysis reagent to inhibit native RNases within the sample (which might otherwise impair target and/or reporter detection downstream). Exemplary RNase inhibitors include RNAse Inhibitor, Murine (NEB), RnaseIn Plus (Promega), Protector Rnase Inhibitor (Roche), SuperaseIn (Ambion), RiboLock (Thermo), Ribosafe (Bioline), or the like. Alternatively, or in combination, when a protease is used for sample lysis, a protease inhibitor can be applied to the lysed sample to inactivate the protease prior to contacting the sample nucleic acids to the programmable nuclease. Additional application of heat may not be required to inhibit the protease (e.g., proteinase K) sufficiently to prevent additional activity of the protease (which could potentially impair programmable nuclease activity downstream, in some embodiments). Exemplary protease inhibitors include AEBSF, antipain, aprotinin, bestatin, chymostatin, EDTA, leupeptin, pepstatin A, phosphoramidon, PMSF, soybean trypsin inhibitor, TPCK, or the like. In some instances, enzyme inactivation may occur before, during, after, or instead of heat inactivation.

In some cases, a target nucleic acid within the sample can undergo amplification before binding to a guide nucleic acid, for example a crRNA of a CRISPR enzyme. The target nucleic acid within a purified sample can be amplified. In some instances, amplification can be accomplished using loop mediated amplification (LAMP), isothermal recombinase polymerase amplification (RPA), and/or polymerase chain reaction (PCR). In some instances, digital droplet amplification can used. Such nucleic acid amplification of the sample can improve at least one of a sensitivity, specificity, or accuracy of the detection of the target RNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes.

In some embodiments, amplification can comprise thermocycling of the sample. Thermocycling can be carried out for one or more droplets of the sample in parallel and/or independently in separate locations. This can be accomplished by methods such as (1) by holding droplets stationary in locations where a heating element is in close proximity to the droplet on one of the droplet sides and a heat sink element is in close proximity to the other side of the droplet, or (2) flowing the droplet through zones in a fluid channel where heat flows across it from a heating source to a heat sink. In some cases, one or more resistive heating elements can be used to perform thermocycling. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 65° C. In some cases, the nucleic acid amplification reaction can be performed at a temperature that ranges from about 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C., from 45° C. to 65° C., from 50° C. to 65° C., from 55° C. to 65° C., from 60° C. to 65° C., from 40° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C.

Additionally, target nucleic acid can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

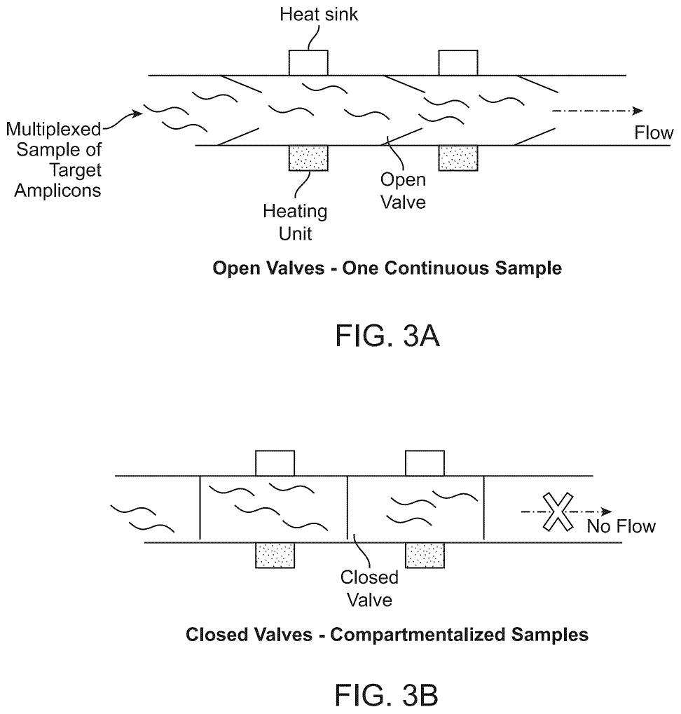

FIG. 3A illustrates an example of a channel through which a continuous flow of a sample can travel to undergo a thermocycling procedure. The device can comprise one or more movable mechanisms that are integrated into the device. The one or more movable mechanisms can be powered using a battery that is integrated with the device. The one or more movable mechanisms can be configured to stop and start the continuous flow of the sample through the channel at one or more predetermined time intervals. The one or more movable mechanisms can be configured to chop or divide the continuous flow of the sample into a plurality of smaller volumes, which can be referred to herein as “droplets.” The one or more movable mechanisms can have an open configuration and a closed configuration. The open configuration can permit a continuous flow of the sample through one or more sections of the channel, as seen in FIG. 3A. The closed configuration can restrict or completely inhibit a flow of the sample through one or more sections of the channel, as seen in FIG. 3B. The closed configuration can permit a physical and/or thermal separation of one or more volumes or portions of the sample flowing through the channel. When the movable mechanisms are in the closed position, the movable mechanisms can provide a physical barrier between different volumes or portions of the sample flowing through the channel. The droplet volume can range from 0.01 to 1 microliter, 1 to 5 microliters and 5 to 50 microliters. The different volumes or partitions can correspond to the droplets described elsewhere herein. The movable mechanisms can switch from the open position to the closed position, or from the closed position to the open position, depending on an operation of a syringe that is providing the sample or another flow regulator that is controlling a flow of the sample through the channel. The movable mechanisms can be powered using a battery that is integrated with the device.

In some cases, the movable mechanism can comprise a plurality of valves. The plurality of valves can comprise, for example, a check valve. In some cases, the movable mechanism can comprise a plunger or a bristle. The plunger or bristle can have an open configuration and a closed configuration. As described above, the open configuration can permit a continuous flow of the sample through one or more sections of the channel, and the closed configuration can restrict or completely inhibit a flow of the sample through one or more sections of the channel. The closed configuration can permit a physical and/or thermal separation of one or more volumes or portions of the sample flowing through the channel. When in the open configuration, the plunger or the bristle can be positioned flush to a bottom of the channel so that the sample can flow through the channel. When in the closed configuration, the plunger or the bristle can be configured to extend from a bottom portion of the channel to a top portion of the channel so that the sample flow is restricted and the sample is divided into a plurality of different droplets, or partitions, that are physically and/or thermally isolated from each other. In some cases, the movable mechanism can comprise any physical object (e.g., a plate) that can be configured to restrict flow through the channel at one or more sections of the channel. In some cases, the movable mechanism can comprise a hinge or spring mechanism to move the movable mechanism between an open configuration and a closed configuration.

The movable mechanisms can be used to generate one or more droplets, aliquots, or subsamples. Each of the one or more droplets, aliquots, or subsamples generated using the movable mechanism can be physically and/or thermally isolated within a plurality of different portions within the channel. The droplets, aliquots, or subsamples can be physically constrained within different portions within the channel. The droplets, aliquots, or subsamples can be constrained between a first movable mechanism that is in a closed position and a second movable mechanism that is in a closed position. The first movable mechanism can be located at a first distance from an inlet of the channel, and the second movable mechanism can be located at a second distance from the inlet of the channel. The channel can be part of a closed system through which the sample can flow. In some cases, when the sample flow through an inlet of the channel is stopped (e.g., a plunger of a syringe containing the sample is pulled back), the one or more movable mechanisms can be placed in a closed configuration, thereby separating the sample already within the channel into a plurality of thermally and physically isolated droplets. Generating droplets, aliquots, or subsamples can simplify the solution, reduce a complexity of the solution, and enhance an accessibility of targets for amplification.

The one or more droplets, aliquots, or subsamples generated using the movable mechanism can undergo an amplification step or a thermocycling step as described elsewhere herein. In some cases, the one or more droplets generated using the movable mechanisms can come into contact with separate heating units and heat sinks while constrained between two movable mechanisms. Different sections of the channel can comprise a plurality of heating units and heat sinks configured to perform thermocycling for different droplets. Individual thermocycling of the droplets, aliquots, or subsamples can permit more efficient thermocycling of smaller volumes of fluid, and can require less energy usage (e.g., from a battery). One or more valves can control a flow or a movement of the sample through the channel. The one or more valves can comprise a check valve that is configured to restrict a movement of the sample or the one or more droplets such that the sample or the one or more droplets do not travel backwards towards an inlet portion of the channel. The one or more valves can control when the sample or the droplets come into thermal contact with the heating unit and/or the heat sink. The timing of such thermal contact can correspond to a timing of one or more thermocycling steps. In some cases, a first droplet of the sample can be in thermal contact with a first heating unit and a first heat sink, a second droplet of the sample can be in thermal contact with a second heating unit and a second heat sink, and so on.

As described above, the devices of the present disclosure can be configured to perform droplet digitization or droplet generation. Droplet digitization or generation can comprise splitting a volume of the sample into multiple droplets, aliquots, or subsamples. The sample can have a volume that ranges from about 10 microliters to about 500 microliters. The plurality of droplets, aliquots, or subsamples can have a volume that ranges from about 0.01 microliters to about 100 microliters. The plurality of droplets, aliquots, or subsamples can have a same or substantially similar volume. In some cases, the plurality of droplets, aliquots, or subsamples can have different volumes. In some cases, the droplets, aliquots, or subsamples can be generated using a physical filter or the one or more movable mechanisms described above. In some cases, each droplet of the sample can undergo one or more sample preparation steps (e.g., nucleic acid purification, lysis, heat inactivation, enzyme inactivation, amplification, etc.) independently and/or in parallel while the droplets are physically constrained or thermally isolated between two movable mechanisms.

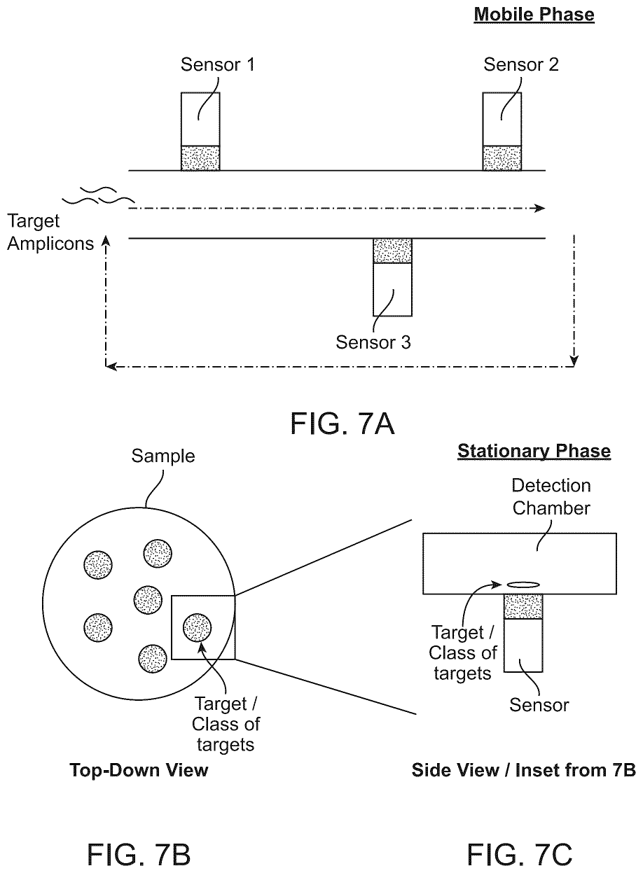

After amplification, the sample can be remixed. The sample can be circulated through the detection chamber using a bulk circulation mechanism that is configured to stir the remixed sample around such that the remixed sample comes into contact with one or more programmable nuclease probes, as shown in FIG. 7A. In some cases, the sample can be provided on a portion of a surface of the detection chamber that is proximal to one or more programmable nuclease probes, as shown in FIG. 7C. In some cases, the detection chamber can be configured to direct the sample along one or more fluid flow paths that position the remixed sample adjacent and/or proximal to one or more programmable nuclease probes. The one or more fluid flow paths can be used to target delivery of at least a portion of the remixed droplets to one or more detection regions associated with the one or more programmable nuclease probes. The remixed droplets can be circulated through the detection chamber along one or more desired fluid flow paths with aid of a piezoelectric device.

In some embodiments, electrowetting can be used by the device for sample transport. In some cases, the device can be configured for electrowetting-on-dielectric (EWOD) applications. The devices of the present disclosure can comprise an array of independently addressable electrodes integrated into the device.

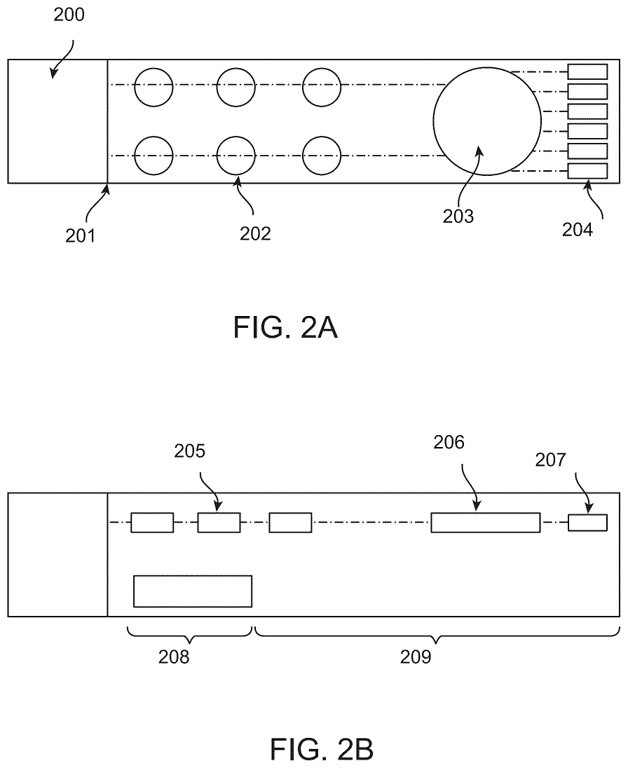

Described herein are various embodiments of a device for programmable nuclease-based (e.g., CRISPR-based) assays. FIG. 2A illustrates a top-down view of an exemplary device for CRISPR-based detection. The device can comprise a sample interface (200) that is configured to receive a sample. The sample can undergo one or more processing steps as described elsewhere herein. Any of the devices described herein may comprise a physical filter (201) to filter one or more particles from the sample that do not comprise the one or more targets (e.g., a gene of interest). In some embodiments, the device may comprise thermocycling components (202). In some cases, the sample can be divided into a plurality of digitized droplets. The plurality of digitized droplets can be provided in a plurality of different chambers of the device. The plurality of digitized droplets can undergo separate processing steps (e.g., thermocycling). In some embodiments, the independent digital reactions can be conducted in parallel with no cross-talk therebetween. In other embodiments, the independent digital reactions can be conducted in parallel with a minimal level or amount of cross-talk. The plurality of droplets can be mixed together after the separate processing steps upon completion of the processing steps. A plurality of programmable nuclease probes comprising one or more programmable nucleases (e.g., Cas enzymes) can be operatively coupled to the detection chamber (203) to detect one or more targets in the sample or the plurality of digitized droplets that are mixed together in the detection chamber. One or more detectors (204) may be configured to detect a signal from the detection chamber as described herein. A side profile of an embodiment of an exemplary programmable nuclease-based device is shown in FIG. 2B. In some embodiments, the device may comprise one or more thermocycling compartments (205) as seen in sideview. In some embodiments, the device may comprise a detection chamber (206). In some embodiments, the device may comprise one or more detectors (207). In some embodiments, the device may comprise a battery (208). In some embodiments, the device may comprise telemedicine components.

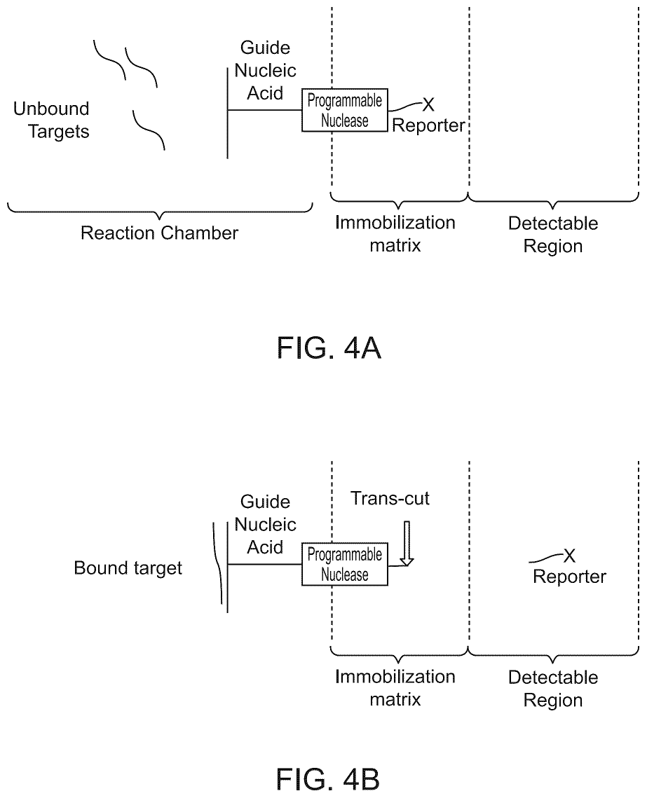

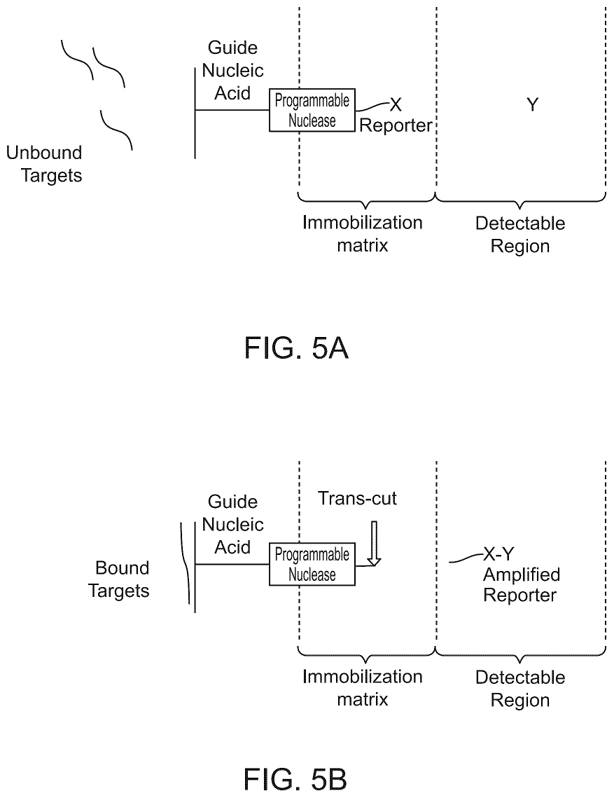

FIGS. 4A, 4B, 5A, and 5B illustrate an exemplary programmable nuclease probe that can be used in a compatible manner with the devices of the present disclosure. The programmable nuclease probe can comprise a guide nucleic acid complexed with a programmable nuclease. The programmable nuclease can comprise any type of programmable nuclease as described herein. In some cases, the programmable nuclease probe may comprise a guide nucleic acid complexed with a CRISPR enzyme. For example, FIG. 4A shows unbound target amplicons in the circulation chamber prior to binding to a guide RNA, which in turn is contacted to a programmable nuclease (e.g., a CRISPR enzyme). The guide RNA-CRISPR enzyme complex also includes a reporter. The programmable nuclease probe (e.g., a CRISPR probe) is immobilized to an immobilization matrix, where the interior side of the immobilization matrix is exposed to the inside wall of the circulation chamber. The guide nucleic acid or guide RNA is exposed to the target amplicons inside the circulation chamber. The reporter is in proximity to the “exterior” side of the immobilization matrix, where the exterior side of the immobilization matrix be in proximity to a detection region. FIG. 4B illustrates a programmable nuclease probe (e.g., a CRISPR probe) after binding with a complementary target amplicon. The binding event triggers a trans-cut that releases the reporter into a detectable region or changes the reporter. Detection mechanisms can involve interferometry, surface plasmon resonance, electrochemical detection such as potentiometry, or other detection mechanisms.

In certain instances, as seen in FIGS. 5A and 5B, the reporter of the programmable nuclease probe can initiate a signal amplification reaction with another molecular species after the complementary binding induced trans-cutting. Such species can be a reactive solid or gel matrix, or other reactive entity to generate an amplified signal during detection. The signal amplification reaction can be physical or chemical in nature. In certain instances, as seen in FIGS. 5A and 5B, after a complementary binding induced trans-cut, the released reporter, X, can initiate an interaction and/or a reaction with another entity, Y to produce an amplified or modified signal. Such entities can comprise a molecular species, a solid, a gel, or other entities. The signal amplification interaction can be a physical or chemical reaction. In some embodiments, the interaction involves free-radical, anionic, cationic or coordination polymerization reactions. In other embodiments the cut reporter can trigger aggregation, or agglutination, of molecules, cells, or nanoparticles. In some instances, the cut reporter can interact with a semiconductor material. In some embodiments, the chemical or physical change caused by the interaction is detected by optical detection means such as interferometry, surface plasmon resonance, reflectivity or other. In other embodiments, the chemical or physical change caused by the interaction is detected by potentiometric, amperometric, field effect transistor, or other electronic means

The programmable nuclease probe can comprise a programmable nuclease and/or a guide nucleic acid. The guide nucleic acid can bind to a target nucleic acid, as described in greater detail below. In some case, to minimize off-target binding (which can slow down detection or inhibit accurate detection), the device can be configured to generate an electro-potential gradient or to provide heat energy to one or more regions proximal to the programmable nuclease probe, to enhance targeting.

In some embodiments, one or more guide nucleic acids can be used to carry out highly efficient, rapid, and accurate reactions for detecting whether a target nucleic acid is present in a sample. The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded target nucleic acid comprising a portion of a nucleic acid from a cancer gene or gene associated with a genetic disorder as described herein. The guide nucleic acid is complementary to the target nucleic acid. Often the guide nucleic acid binds specifically to the target nucleic acid. The target nucleic acid can be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid may comprise a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid can have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30 nt to about 40 nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of HPV 16 or HPV 18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling may comprise gaps between the tiled guide nucleic acids along the target nucleic acid. In some instances, the tiling of the guide nucleic acids is non-sequential. Often, a method for detecting a target nucleic acid may comprise contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that can be caused by multiple organisms.

In some embodiments, programmable nucleases can be used to carry out highly efficient, rapid, and accurate reactions for detecting whether a target nucleic acid is present in a sample. A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. A signal can be a calorimetric, potentiometric, amperometric, optical (e.g., fluorescent, colorimetric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid may comprise a crRNA and a trans-activating crRNA (tracrRNA).

The programmable nuclease system used to detect modified target nucleic acids can comprise CRISPR RNAs (crRNAs), trans-activating crRNAs (tracrRNAs), Cas proteins, and detector nucleic acids.

Described herein are reagents comprising a programmable nuclease capable of being activated when complexed with the guide nucleic acid and the target nucleic acid segment or portion. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas protein (also referred to, interchangeably, as a Cas nuclease). A crRNA and Cas protein can form a CRISPR enzyme.

Several programmable nucleases are consistent with the methods and devices of the present disclosure. For example, CRISPR/Cas enzymes are programmable nucleases used in the methods and systems disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type V1 CRISPR/Cas enzymes. Preferable programmable nucleases included in the several devices disclosed herein (e.g., a microfluidic device such as a pneumatic valve device or a sliding valve device or a lateral flow assay) and methods of use thereof include a Type V or Type V1 CRISPR/Cas enzyme.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acid via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In some instances, a programmable Cas 12 nuclease can be a Cas12a (also referred to as Cpf1) protein, a Cas12b protein, Cas 12c protein, Cas12d protein, or a Cas12e protein.

In some embodiments, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the Cas12 can be a Cas12 variant having the sequence of SEQ ID NO: 17 (or a sequence with at least 60%, 70%, 80%, 90%, or 95% identity thereto). In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type V1 CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target DNA to initiate trans cleavage of an RNA reporter, such as a Type V1 CRISPR/Cas enzyme (e.g., a Cas13 nuclease). For example, Cas 13a of the present disclosure can be activated by a target RNA to initiate trans cleavage activity of the Cas13a for the cleavage of an RNA reporter and can be activated by a target DNA to initiate trans cleavage activity of the Cas 13a for trans cleavage of an RNA reporter. An RNA reporter can be an RNA-based reporter. In some embodiments, the Cas13a recognizes and detects ssDNA to initiate transcleavage of RNA reporters. Multiple Cas13a isolates can recognize, be activated by, and detect target DNA, including ssDNA, upon hybridization of a guide nucleic acid with the target DNA. For example, Lbu-Cas13a and Lwa-Cas13a can both be activated to transcollaterally cleave RNA reporters by target DNA. Thus, Type V1 CRISPR/Cas enzyme (e.g., a Cas13 nuclease, such as Cas13a) can be DNA-activated programmable RNA nucleases, and therefore can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly taken from organisms or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type V1 protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example, target ssDNA detection by Cas13a can be employed in a detection device as disclosed herein.

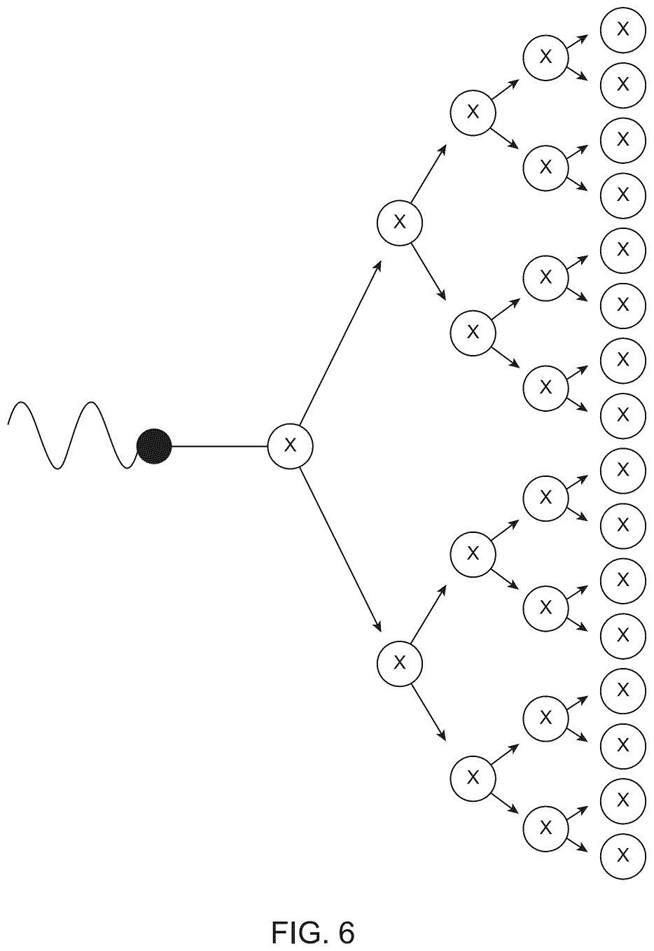

In any of the embodiments described herein, the programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid, which can initiate trans cleavage activity. In some cases, the trans cut or trans cleavage can cut and/or release a reporter. In other cases, the trans cut or trans cleavage can produce an analog of a target, which can be directly detected. Trans cleavage activity can be non-specific cleavage of nearby nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. For example, the detection moiety can correspond to the element, or moiety, (X) shown in FIG. 4A, 4B, 5A, 5B, and FIG. 6. The signal can be immobilized on a support medium for detection. The signal can be visualized to assess whether a target nucleic acid is present or absent.

Reporters, which can be referred to interchangeably as reporters or detector nucleic acids, can be used in conjunction with the compositions disclosed herein (e.g., programmable nucleases, guide nucleic acids, etc.) to carry out highly efficient, rapid, and accurate reactions for detecting whether a target nucleic acid is present in a sample. The reporter can be suspended in solution or immobilized on a surface. For example, the reporter can be immobilized on the surface of a chamber in a device as disclosed herein. In some cases, the reporter can be immobilized on beads, such as magnetic beads, in a chamber of a device as disclosed herein where they are held in position by a magnet placed below the chamber. The reporter can be capable of being cleaved by the activated programmable nuclease, thereby generating a detectable signal. The detectable signal can correspond to a release of one or more elements (X) as illustrated in FIGS. 5A and 5B. The release of the one or more elements (X) can initiate a reaction with another element (Y) when the element (Y) is in the presence of the element (X). The reaction between the element (Y) and the element (X) can initiate a chemical chain reaction in a solid phase material. Such a chemical chain reaction can produce one or more physical or chemical changes. In some cases, the physical or chemical changes can be optically detected. In some embodiments, one or more cascade amplification reactions can occur to further amplify the signal before sensing or detection. There can be a single point of attachment between the reporter and the element (X). Cutting the single point of attachment can release a macro molecule (X), which can undergo a series of reactions based on the macro molecule (X) itself. In any of the embodiments described herein, the reporter can comprise a single stranded detector nucleic acid comprising a detection moiety.

As used herein, a detector nucleic acid is used interchangeably with reporter or reporter. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid may comprise at least 2, 3, 4, 5, 6, 7, 8, 9, or 10 ribonucleotide residues at an internal position. In some cases, the detector nucleic acid may comprise from 2 to 10, from 3 to 9, from 4 to 8, or from 5 to 7 ribonucleotide residues at an internal position. In some cases, the detector nucleic acid may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid may comprise nucleotides resistant to cleavage by the programmable nuclease described herein. In some cases, the detector nucleic acid may comprise synthetic nucleotides. In some cases, the detector nucleic acid may comprise at least one ribonucleotide residue and at least one non-ribonucleotide residue. In some cases, the detector nucleic acid is 5-20, 5-15, 5-10, 7-20, 7-15, or 7-10 nucleotides in length. In some cases, the detector nucleic acid is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the detector nucleic acid may comprise at least one uracil ribonucleotide. In some cases, the detector nucleic acid may comprise at least two uracil ribonucleotides. Sometimes the detector nucleic acid has only uracil ribonucleotides. In some cases, the detector nucleic acid may comprise at least one adenine ribonucleotide. In some cases, the detector nucleic acid may comprise at least two adenine ribonucleotides. In some cases, the detector nucleic acid has only adenine ribonucleotides. In some cases, the detector nucleic acid may comprise at least one cytosine ribonucleotide. In some cases, the detector nucleic acid may comprise at least two cytosine ribonucleotides. In some cases, the detector nucleic acid may comprise at least one guanine ribonucleotide. In some cases, the detector nucleic acid may comprise at least two guanine ribonucleotides. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

The single stranded detector nucleic acid can comprise a detection moiety capable of generating a first detectable signal. Sometimes the detector nucleic acid may comprise a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single stranded nucleic acid capable of generating a first detectable signal. Optionally, there are more than one population of single-stranded detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded detector nucleic acids capable of generating a detectable signal. In some cases, there are from 2 to 50, from 3 to 40, from 4 to 30, from 5 to 20, or from 6 to 10 different populations of single-stranded detector nucleic acids capable of generating a detectable signal. In some cases there are from 2 to 50, from 5 to 50, from 10 to 50, from 15 to 50, from 20 to 50, from 25 to 50, from 30 to 50, from 35 to 50, from 40 to 50, from 2 to 40, from 5 to 40, from 10 to 40, from 15 to 40, from 20 to 40, from 25 to 40, from 30 to 40, from 35 to 40, from 2 to 30, from 5 to 30, from 10 to 30, from 15 to 30, from 20 to 30, from 25 to 30, from 2 to 20, from 5 to 20, from 10 to 20, from 15 to 20, from 2 to 10, or from 5 to 10 different populations of single-stranded detector nucleic acids capable of generating a detectable signal.

In some embodiments, target nucleic acid amplicons are detected by immobilized programmable nuclease probes, such as, for example, CRISPR CAS guide RNA probes (referred to as CRISPR probe). Upon a complementary binding event between a target nucleic acid amplicon and a programmable nuclease probe (e.g., an immobilized CRISPR CAS/guide RNA complex) a cutting event will occur that release a reporter that is then detected by a sensor. There are two main schemes for detection of the binding events between the target and the programmable nuclease probe, a mobile phase scheme as illustrated in FIG. 7A and a stationary phase scheme as illustrated in FIG. 7C. In any of the embodiments described herein, each programmable nuclease probe can have one or more programmable nucleases (e.g., CRISPR enzymes) with one or more specific guide RNAs for detecting different target nucleic acids or different classes of target nucleic acids.

In certain circumstances, a mobile phase detection scheme (FIG. 7A) is used. In these circumstances, the remixed sample is flown through a channel. The walls of the channel can comprise one or more regions on which one or more programmable nuclease probes or CRISPR probes are disposed. The programmable nuclease probes or CRISPR probes can comprise a guide nucleic acid (e.g., a guide RNA) complexed with a programmable nuclease as described elsewhere herein. The guide nucleic acid and/or the programmable nuclease can be immobilized relative to the walls of the channel. The guide nucleic acid and/or the programmable nuclease can be exposed to the remixed sample flowing through the channel. Reporter compounds can be cut and released upon a complementary binding event of a target nucleic acid amplicon and a specific guide nucleic acid. The reporter compounds can freely participate in one or more cascading amplification reactions that generate an amplified signal, as illustrated in FIG. 6. The amplified signal can be detected using one or more sensors as described herein.

In some embodiments, a stationary phase detection scheme is used. In these embodiments the remixed sample can contain one or more target nucleic acid amplicon sequence types and copies thereof. FIG. 7B shows a top-down view of a plurality of target amplicons that can interact with programmable nuclease probes positioned at known locations on the interior walls of the reaction chamber. FIG. 7C shows a cross-sectional side view of the detection chamber with a target amplicon interacting with a programmable nuclease probe. The guide RNA of the programmable nuclease or CRISPR probe can be immobilized adjacent to a bottom surface of the chamber. When a complementary interaction between the probe and the target occurs, the CRISPR enzyme will cut and release a reporter which will then be sensed. Since the specific guide RNA of the immobilized programmable nuclease or CRISPR probe can be spatially registered, multiplexed detection can be achieved. In some cases, where one sensor corresponds to one immobilized probe, electrical detection can be used. Other methods of detection can also be used, such as optical imaging, surface plasmon resonance (SPR), and/or interferometric sensing.

The plurality of programmable nuclease probes shown in FIG. 2, FIG. 7A, and FIG. 7C and described herein can be arranged in various configurations. For example, the plurality of programmable nuclease probes can be arranged in a lateral configuration. Alternatively, the plurality of programmable nuclease probes can be arranged in a circular configuration such that each programmable nuclease probe is equidistant from a common center point. In some cases, the plurality of programmable nuclease probes can be distributed with a same separation distance or spacing between the programmable nuclease probes. In other cases, a first programmable nuclease probe and a second programmable nuclease probe can be separated by a first separation distance or spacing, and a third programmable nuclease probe and a fourth programmable nuclease probe can be separated by a second separation distance or spacing that is different than the first separation distance or spacing.

As described above, the single stranded detector nucleic acid can comprise a detection moiety capable of generating a first detectable signal. Sometimes the detector nucleic acid may comprise a protein capable of generating a signal. A signal can be a colorimetric, potentiometric, amperometric, optical (e.g., fluorescent, colorometric, etc.), or piezo-electric signal. The generation of the detectable signal from the release of the detection moiety indicates that cleavage by the programmable nuclease has occurred and that the sample contains the target nucleic acid. A detection moiety can be any moiety capable of generating a colorimetric, potentiometric, amperometric, optical (e.g., fluorescent, colorometric, etc.), or piezo-electric signal. A detector nucleic acid, sometimes, is protein-nucleic acid that can generate a calorimetric, potentiometric, amperometric, optical (e.g., fluorescent, colorometric, etc.), or piezo-electric signal upon cleavage of the nucleic acid. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid.

Detecting the presence or absence of a target nucleic acid of interest can involve measuring a signal emitted from a detection moiety present in a reporter, after cleavage of the reporter by an activated programmable nuclease. The signal can be measured using one or more sensors integrated with the device or operatively coupled to the device. Thus, the detecting steps disclosed herein can involve measuring the presence of a target nucleic acid, quantifying how much of the target nucleic acid is present, or, measuring a signal indicating that the target nucleic acid is absent in a sample. In some embodiments, a signal is generated upon cleavage of the detector nucleic acid by the programmable nuclease. In other embodiments, the signal changes upon cleavage of the detector nucleic acid by the programmable nuclease. In other embodiments, a signal can be present in the absence of detector nucleic acid cleavage and disappear upon cleavage of the target nucleic acid by the programmable nuclease. For example, a signal can be produced in a microfluidic device or lateral flow device after contacting a sample with a composition comprising a programmable nuclease.

In some cases, the signal can comprise a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system can detect more than one type of target nucleic acid, wherein the system may comprise more than one type of guide nucleic acid and more than one type of detector nucleic acid. In some cases, the detectable signal is generated directly by the cleavage event. Alternatively, or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium.

In some cases, the one or more detectable signals generated after cleavage can produce an index of refraction change or one or more electrochemical changes. In some cases, real-time detection of the Cas reaction can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope).

In certain aspects of this disclosure, multiplexing refers to parallel sensing of multiple target nucleic acid sequences in one sample by multiple probes.

FIGS. 7A, 7B, and 7C illustrate two multiplexing embodiments of a CRISPR detection device, as described herein. FIGS. 7A, 7B, and 7C illustrate two multiplexing embodiments of the CRISPR detection device. FIG. 7A illustrates a capillary flow or mobile sample phase embodiment and FIGS. 7B and 7C illustrate a stationary sample phase embodiment. FIG. 7A illustrates, in certain embodiments, a reaction chamber that is in the form of a capillary circuit. Functionalized programmable nuclease probes, e.g., CRISPR probes, can be disposed on the capillary walls, and one or more guide nucleic acids associated with the programmable nuclease probes, e.g., CRISPR probes can be exposed to the sample for binding. Upon binding to a complementary target nucleic acid amplicon (or a target nucleic acid sequence) the programmable nuclease probe or CRISPR probe then cuts and releases at least a portion of a reporter that generates a signal indicating the presence of the particular target nucleic acid amplicon. This identification process is repeated in parallel across multiple programmable nuclease probes or CRISPR probes, where each programmable nuclease or CRISPR probe is configured to detect a particular target sequence, nucleic acid amplicon, set of target sequences, or set of target nucleic acid amplicons. In certain aspects, as seen in FIGS. 7B and 7C, multiplexed detection can also be achieved in a stationary phase, or microarray format. In some embodiments, programmable nuclease probes or CRISPR probes, each designed to detect certain target nucleic acid sequences, are immobilized in known locations. When the remixed sample containing multiple types of target amplicons is exposed to the array of programmable nuclease or CRISPR probes, the specific probe-target pairs will bind and trigger signal events. These signal events can be associated with a particular target nucleic acid amplicon or set of target nucleic acid amplicons either by its location when imaging is used, or by a signal received by a particular sensor when sensors are individually linked to each probe. In some instances, one or more target nucleic acid amplicons can be detected by a programmable nuclease probe. In some instances, the programmable nuclease probe can interact with and/or detect a class of sequences or a class of target nucleic acid amplicons, which can indicate a presence or an existence of a particular organism, disease state, or phenotype present within the sample.

The devices of the present disclosure can be used to implement for detection of one or more target nucleic acids within the sample. The devices of the present disclosure can comprise one or multiple pumps, valves, reservoirs, and chambers for sample preparation, optional amplification of a target nucleic acid within the sample, mixing with a programmable nuclease, and detection of a detectable signal arising from cleavage of detector nucleic acids by a programmable nuclease.

Methods consistent with the present disclosure include a multiplexing method of assaying for a plurality of target nucleic acids in a sample. A multiplexing method may comprise contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some reporters (e.g., protein-nucleic acids) of a population of reporter molecules (e.g., protein-nucleic acids), wherein the signal indicates a presence of the target nucleic acid in the sample and wherein absence of the signal indicates an absence of the target nucleic acid in the sample.

Multiplexing can comprise spatial multiplexing wherein multiple different target nucleic acids are detected at the same time, but the reactions are spatially separated. In some cases, the multiple target nucleic acids are detected using the same programmable nuclease, but different guide nucleic acids. The multiple target nucleic acids sometimes are detected using the different programmable nucleases. In the case wherein multiple target nucleic acids are detected using the different programmable nucleases, the method involves using a first programmable nuclease, which upon activation (e.g., after binding of a first guide nucleic acid to a first target), cleaves a nucleic acid of a first reporter and using a second programmable nuclease, which upon activation (e.g., after binding of a second guide nucleic acid to a second target), cleaves a nucleic acid of a second reporter.

Sometimes, multiplexing can be single reaction multiplexing wherein multiple different target acids are detected in a single reaction volume. Often, at least two different programmable nucleases are used in single reaction multiplexing. For example, multiplexing can be enabled by immobilization of multiple categories of detector nucleic acids within a fluidic system, to enable detection of multiple target nucleic acids within a single fluidic system. Multiplexing allows for detection of multiple target nucleic acids in one kit or system. In some cases, the multiple target nucleic acids comprise different target nucleic acids to a virus, a bacterium, or a pathogen responsible for one disease. In some cases, the multiple target nucleic acids comprise different target nucleic acids associated with a cancer or genetic disorder. Multiplexing for one disease, cancer, or genetic disorder increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. Multiplexing, thus, allows for multiplexed detection of multiple genomic alleles. For example, multiplexing may comprise method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different HPV strains, for example, HPV16 and HPV18. In some cases, the multiple target nucleic acids comprise target nucleic acids directed to different cancers or genetic disorders. Often, multiplexing allows for discrimination between multiple target nucleic acids, such as target nucleic acids that comprise different genotypes, for example, for a wild-type genotype and for SNP genotype. Multiplexing for multiple diseases, cancers, or genetic disorders provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel may comprise assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. In this context, a unique target nucleic acid refers to the sequence of a nucleic acid that has an at least one nucleotide difference from the sequences of the other nucleic acids in the plurality. Multiple copies of each target nucleic acid can be present. For example, a unique target nucleic population can comprise multiple copies of the unique target nucleic acid. Often the plurality of unique target nucleic acids is from a plurality of bacterial pathogens in the sample.

In some instances, the multiplexed devices, systems, fluidic devices, kits, and methods detect at least 2 different target nucleic acids in a single reaction. In some instances, the multiplexed devices, systems, fluidic devices, kits, and methods detect at least 3 different target nucleic acids in a single reaction. In some instances, the multiplexed devices, systems, fluidic devices, kits, and methods detect at least 4 different target nucleic acids in a single reaction. In some instances, the multiplexed devices, systems, fluidic devices, kits, and methods detect at least 5 different target nucleic acids in a single reaction. In some cases, the multiplexed devices, systems, fluidic devices, kits, and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit. In some instances, the multiplexed kits detect from 2 to 10, from 3 to 10, from 4 to 10, from 5 to 10, from 6 to 10, from 7 to 10, from 8 to 10, from 9 to 10, from 2 to 9, from 3 to 9, from 4 to 9, from 5 to 9, from 6 to 9, from 7 to 9, from 8 to 9, from 2 to 8, from 3 to 8, from 4 to 8, from 5 to 8, from 6 to 8, from 7 to 8, from 2 to 7, from 3 to 7, from 4 to 7, from 5 to 7, from 6 to 7, from 2 to 6, from 3 to 6, from 4 to 6, from 5 to 6, from 2 to 5, from 3 to 5, from 4 to 5, from 2 to 4, from 3 to 4, or from 2 to 3 different target nucleic acids in a single kit. Multiplexing can be carried out in a single-pot or “one-pot” reaction, where reverse transcription, amplification, in vitro transcription, or any combination thereof, and detection are carried out in a single volume. Multiplexing can be carried out in a “two-pot reaction”, where reverse transcription, amplification, in vitro transcription, or any combination thereof, are carried out in a first volume and detection is carried out in a second volume.

In some cases, multiplexing can comprise detecting multiple targets with a single probe. Alternatively, multiplexing can comprise detecting multiple targets with multiple probes. The multiple probes can be configured to detect a presence of a particular sequence, target nucleic acid, or a plurality of different target sequences or nucleic acids.

The devices of the present disclosure can be manufactured from a variety of different materials. Exemplary materials that can be used include plastic polymers, such as poly-methacrylate (PMMA), cyclic olefin polymer (COP), cyclic olefin copolymer (COC), polyethylene (PE), high-density polyethylene (HDPE), polypropylene (PP); glass; and silicon. Features of the device (e.g., chambers, channels, etc.) can be manufactured by various processes. For example, the features can be (1) embossed using injection molding, (2) micro-milled or micro-engraved using computer numerical control (CNC) micromachining or non-contact laser drilling (by means of a CO2 laser source); (3) generated using additive manufacturing, and/or (4) generated using one or more photolithographic or stereolithographic methods.

In some embodiments, any of the devices of the present disclosure can comprise a sample interface configured to receive a sample that may comprise at least one gene of interest. The device can further comprise a channel in fluid communication with the sample interface and a detection chamber. In some cases, the channel may comprise one or more movable mechanisms to separate the sample into a plurality of droplets. As used herein, a droplet can refer to a volumetric portion of the sample, a partitioned sub-sample of the sample, and/or an aliquot of the sample. In some cases, the detection chamber is configured to receive and contact the plurality of droplets with at least one programmable nuclease probe disposed on a surface of said detection chamber. The at least one programmable nuclease probe can comprise a guide nucleic acid complexed with a programmable nuclease. In some cases, the programmable nuclease probe may comprise a CRISPR/Cas enzyme. In some cases, the guide nucleic acid may comprise a guide RNA. In some embodiments, the device may comprise a plurality of programmable nuclease probes comprising different guide RNAs.

The device can further comprise a plurality of sensors that determine a presence of said at least one gene of interest by detecting a signal produced upon cleavage of a target nucleic acid region in said at least one gene of interest by said at least one programmable nuclease probe. The cleavage of the target nucleic acid region can occur after a complementary binding of said target nucleic acid region to said guide nucleic acid of said at least one programmable nuclease probe.

As described elsewhere herein, the one or more movable mechanisms can comprise one or more valves configured to restrict flow through one or more sections of the channel. The one or more movable mechanisms can comprise a plunger or a bristle that is configured to restrict flow through one or more sections of the channel. The one or more movable mechanisms can be operatively coupled to a power source that is integrated with or insertable into the device. The power source can comprise a battery.

In some cases, any of the devices described herein may comprise a physical filter to filter one or more particles from the sample that do not comprise the one or more targets (e.g., a gene of interest). In some cases, the device may comprise a sample preparation chamber. The sample preparation chamber can comprise a lysing agent. The sample preparation chamber can comprise a heating unit configured for heat inactivation. The sample preparation chamber can comprise one or more reagents for nucleic acid purification.

In some cases, the channel between the sample interface and the detection chamber may comprise a plurality of heating elements and a plurality of heat sinks for amplifying the at least one gene of interest or a portion thereof. The plurality of heating elements and the plurality of heat sinks can be configured to perform one or more thermocycling operations on the sample or at least a portion of the sample (e.g., the plurality of droplets).

As described elsewhere herein, the signal produced upon cleavage of a target nucleic acid can be associated with a physical, chemical, or electrochemical change or reaction. The signal can comprise an optical signal, a fluorescent or colorimetric signal, a potentiometric or amperometric signal, and/or a piezo-electric signal. In some cases, the signal is associated with a change in an index of refraction of a solid or gel volume in which the at least one programmable nuclease probe is disposed.

In some embodiments, the device may comprise a sample interface configured to receive a sample that may comprise one or more genomic targets of interest. In some cases, the one or more genomic targets of interest comprise a sequence of nucleic acids comprising the nucleic acid.

The device can further comprise one or more channels comprising one or more movable mechanisms to separate the sample into partitioned samples. The one or more channels can be in fluid communication with the sample interface and a reaction chamber that is configured to receive and contact the partitioned samples with an enzyme, reagent, or programmable detection agent that is configured to cleave a nucleic acid of said one or more genomic targets of interest.

The device can further comprise a plurality of sensors for determining a presence of the one or more genomic targets of interest by detecting one or more reporters released by said cleavage of said nucleic acid. The programmable detection agent can be a CRISPR/Cas enzyme. In some cases, the reporter may comprise a nucleic acid and a detection moiety. In some cases, the reporter may comprise at least one ribonucleotide or at least one deoxyribonucleotide. In some cases, the reporter may comprise a DNA nucleic acid or an RNA nucleic acid. The reported molecule can be immobilized on a surface of the detection chamber (i.e., a movement of the reporter can be physically or chemically constrained).

In some cases, the one or more movable mechanisms comprise a plurality of valves configured to restrict flow in a first direction through the one or more channels towards the sample interface. The plurality of valves can be configured to selectively permit flow in a second direction through the one or more channels towards the reaction chamber. A first valve and a second valve of the plurality of valves can be configured to physically, fluidically, or thermally isolate a first portion of the sample from a second portion of the sample when the first valve and the second valve are in a closed state.

The one or more channels can comprise a plurality of heating elements and a plurality of heat sinks to perform thermocycling on the partitioned samples. A first heating element of the plurality of heating elements and a first heat sink of the plurality of heat sinks can be positioned between a first movable mechanism and a second movable mechanism of the one or more movable mechanisms.

In any of the embodiments described herein, the device can further comprise a telemedicine unit configured to provide one or more detection results to a computing unit that is remote from the device. In some embodiments, the telemedicine unit provides one or more detection results to a computing unit that is remote to the device through a cloud-based connection. In some embodiments, the telemedicine unit is HIPAA compliant. In some embodiments, the telemedicine unit transmits encrypted data. The computing unit can comprise a mobile device or a computer. The one or more detection results can indicate a presence or an absence of a target nucleic acid of interest in the sample.

In another aspect, the present disclosure provides a method for target detection. The method can comprise contacting a sample with the device of any of the preceding claims and detecting a presence or an absence of one or more genes of interest in said sample. In some cases, the method can comprise generating one or more detection results indicating the presence or the absence of the one or more genes of interest in the sample. In some cases, the method can comprise transmitting the one or more detection results to a remote computing unit. The remote computing unit can comprise, for example, a mobile device.

In another aspect, the present disclosure provides a method for target detection. The method can comprise providing a sample comprising at least one gene of interest. The method can comprise separating the sample into a plurality of sub-samples using one or more movable mechanisms described herein. The method can comprise receiving the plurality of sub-samples in a detection chamber and contacting the plurality of sub-samples with at least one programmable nuclease probe disposed on a surface of said detection chamber. The at least one programmable nuclease probe can comprise a guide nucleic acid complexed with a programmable nuclease. In some cases, the method can comprise contacting the plurality of sub-samples with a plurality of programmable nuclease probes comprising different guide RNAs. The method can comprise using a plurality of sensors to determine a presence or an absence of said at least one gene of interest by detecting a signal produced upon cleavage of a target nucleic acid region in said at least one gene of interest by said at least one programmable nuclease probe.

In some cases, the method can further comprise amplifying the at least one gene of interest after separating the sample into a plurality of sub-samples. In some cases, the method can further comprise amplifying the at least one gene of interest before mixing the plurality of sub-samples in the detection chamber. Amplifying the at least one gene of interest can comprise using a plurality of heating elements and a plurality of heat sinks to perform thermocycling on the plurality of sub-samples.

In some cases, the method can comprise using a physical filter to filter one or more particles from the sample that do not comprise the one or more target genes of interest. In some cases, the method can comprise lysing the sample before detecting the one or more target genes of interest. In some cases, the method can comprise performing heat inactivation on the sample. In some cases, the method can comprise performing enzyme (e.g., PK) inactivation on the sample. In some cases, the method can comprise performing nucleic acid purification on the sample.

In some cases, the detection devices described herein can be configured to implement process control procedures to ensure that the sample preparation, target amplification, and target detection processes are performed accurately and as intended.

### Disposable Fluidic Workflow

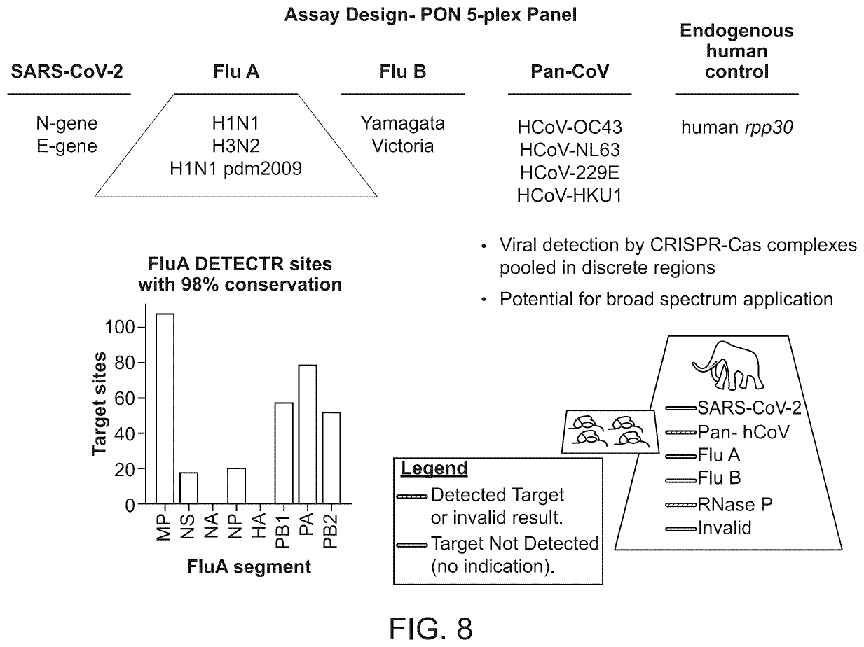

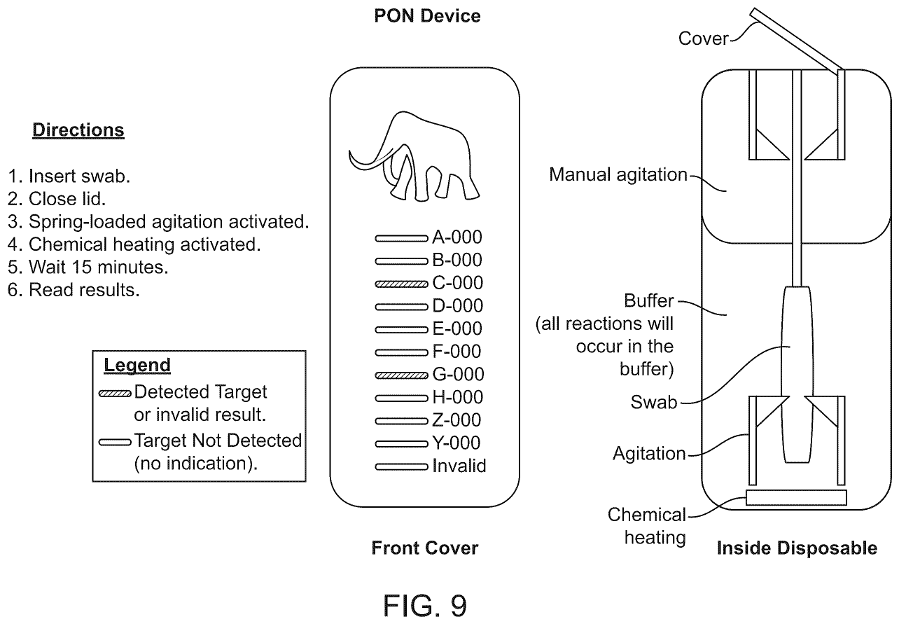

Described herein are various embodiments, for point of need (PON) programmable nuclease-based devices. In some embodiments, the PON device is configured for a 5-plex respiratory panel as shown in FIG. 8. FIG. 8 shows an exemplary assay design for a PON 5-plex panel comprising pooled CRISPR-Cas complexes in discrete regions for viral detection. The discrete regions are for detection of: (1) SARS-CoV-2, (2) Flu A, (3) Flu B, (4) Pan-CoV, and (5) Endogenous human control. The (1) SARS-CoV-2 region may comprise gRNA for detecting N-gene targets and E-gene targets, the (2) Flu A region may comprise gRNA for detecting H1N1 targets, H3N2 targets, and H1N1 pdm2009 targets, the (3) Flu B region may comprise gRNA for detecting Yamagata targets and Victoria targets, the (4) Pan-CoV region may comprise gRNA for detecting HCoV-OC43 targets, HCoV-NL63 targets, HCoV-229E targets, and HCoV-HKU1 targets, and the (5) Endogenous human control region may comprise gRNA for human rpp30 targets. Each region can comprise pooled gRNA. For example, the gRNAs for the Flu A region bind to target sites that are 98% conserved among H1N1, H3N2, and H1N1 pdm2009, such as Matrix Protein 1 (MP), Nonstructural Protein 1 (NS), Neuraminidase (NA), Nucleoprotein (NP), Hemagglutinin (HA), PB1, Polymerase Acidic Protein (PA), and Polymerase Basic Protein 2 (PB2). Detected signal from each region can indicate the detection of a target within that region. In some embodiments, PON programmable nuclease-based devices are disposable, as shown in FIG. 9.

Described herein are various Point-of-need (PON) diagnostics that can rapidly identify causes of ailments on location where needed by patients. In some embodiments, disposable PON devices can be manufactured and delivered to the user in a sterile condition to help fill this need. However, in some embodiments, transferring assays from the lab to a point-of-need device can be a challenge. In some embodiments, integrating a fluidic system capable of delivering rapid, reliable, and easy to interpret results while still being disposable is especially challenging.

Disclosed herein are various embodiments of methods, devices, and compositions for a disposable fluidic workflow. In some embodiments, the workflow method may comprise: (1) sample collection from the patient and delivery to the device, (2) lysis, (3) amplification, and (4) detection/readout. In some embodiments, any of the disposable fluidic devices described herein, may comprise an exterior housing and an interior control printed circuit board (PCB) on which to mount sub-components. In some embodiments, such subcomponents may comprise a swab to collect a sample from patient, a mechanism for extracting sample from the swab (e.g., a scraper), fluidics configured to move the sample within the device, one or more sample chambers, one or more reagent storage bags or chambers, amplification mechanisms, detectors, valves and heating elements. In some embodiments, described herein, valves may be rotary valves capable of diverting sample to and from one chamber or channel to multiple other chambers or channels.

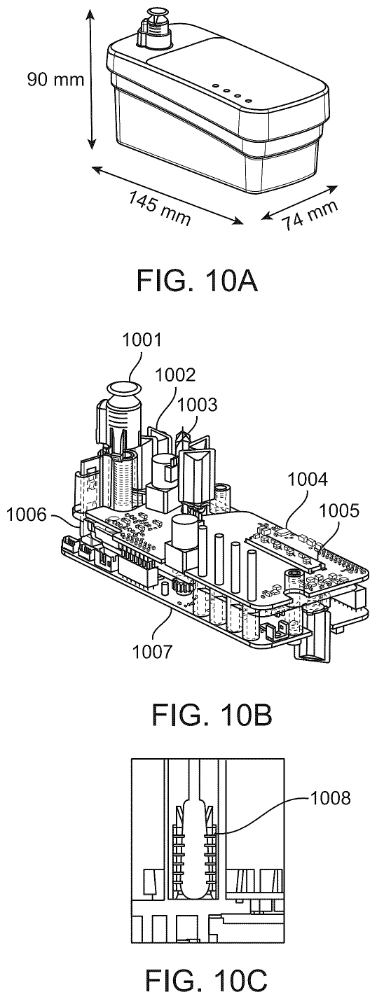

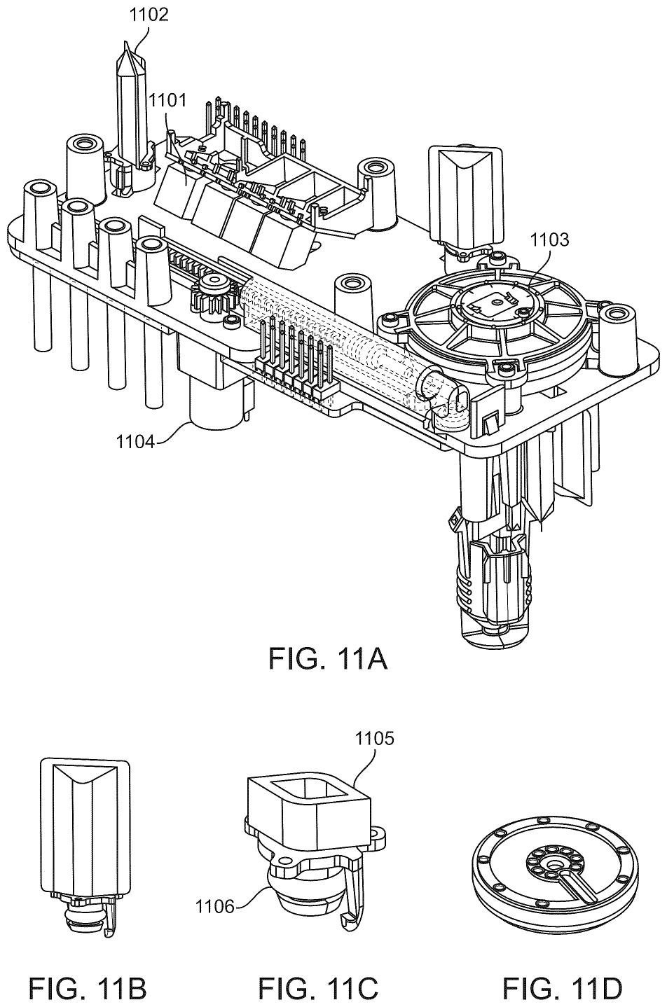

FIG. 10A illustrates an example of a consumable exterior housing. FIG. 10B illustrates an example of components mounted on a printed circuit board (PCB) 1007 and housed within the consumable device housing of FIG. 10A. In some embodiments, a swab cap 1001 may be configured (e.g., rotated, depressed, etc.) to allow user to activate the device. In some embodiments, reagent storage bags (or chambers, etc.) 1002 may be mounted vertically on the PCB 1007. In some embodiments, a motor 1003 to drive the valve, seen in FIG. 11D, may be integrated onto the PCB 1007. In some embodiments, heat traces 1004 may be integrated onto the PCB 1007 to facilitate heating of the sample or a portion thereof. In some embodiments, a light emitting diode (LED) 1005 for excitation of a reporter (e.g., one or more fluorescent labels) may be integrated onto the PCB. In some embodiments, a start button may be activated by pushing on the swab cap 1006. FIG. 10C illustrates an example swab housing, containing a scraper configured to facilitate removal of a sample from the swab 1008. In some embodiments, the sample interface may comprise a scraper. FIG. 9 shows an embodiment of a device comprising a scraper that agitates a swab carrying the sample when the swab is inserted into the inside disposable. FIG. 10C shows another embodiment of a device comprising a scraper (1008). Those skilled in the art will recognize that the scraper may comprise a variety of forms, shapes, and sizes while retaining its function of agitating an input sample. In some embodiments, the scraper facilitates the transfer of sample from the swab to the device.

FIGS. 11A, 11B, 11C, 11D further illustrate various example components of a consumable, as described herein. FIG. 11A illustrates an example of photodiodes for emission detection 1101, expansion chamber bags 1102, encoder PCB 1103, and a syringe pump, which may be disposed in a layer on top of the PCB. In some embodiments, the syringe pump may be driven by a motor 1004. In other embodiments, the syringe pump is driven by a shape memory alloy (SMA) (e.g., a compressed spring) for mass production. FIG. 11B illustrates an exemplary expansion bag 1102. FIG. 11C illustrates an exemplary injection molded bag core of the expansion bag 1102, where a filter (not shown) can be heat sealed on a proximal end thereof 1105. In some embodiments, an O-Ring seal may be made with the injection molded bag core 1106 at a distal end thereof to facilitate coupling of the expansion bag (1102) to the device during manufacture. In other embodiments, an overmold component 1106, or soft polymer such as TPE, may be integrated with the injection molded bag core for mass production. FIG. 11D illustrates an embodiment of a rotatory valve overmold configured to regulate fluid flow within the device as described herein.

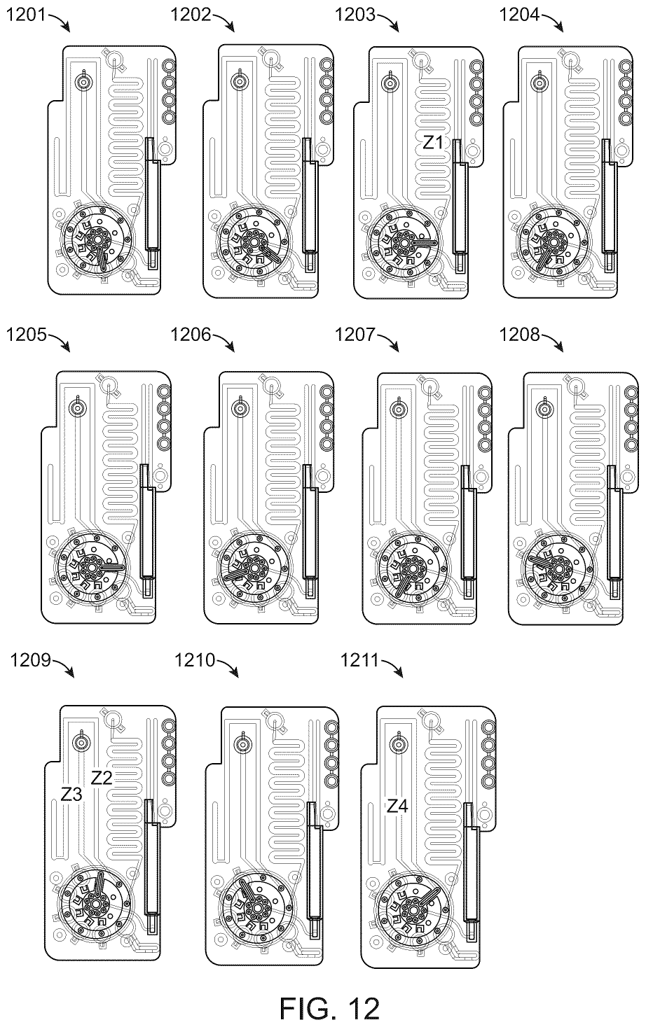

FIG. 12 illustrates various valve position examples of the consumable device in use during the workflow method as follows: extract lysis (1201); transfer lysis swab to extract sample (1202); move lysis to heat zone (Z1) (1203); move lysis to beads for binding (1204); move lysis to waste (1205); extract elution (1206); move elution to beads (1207) move elution to MM (1208); move to PCR cycling (1209); move to dilution (1210); move to DETECTR (1211). FIG. 12 additionally illustrates an embodiment of the consumable with heat zones (Z) for lysis heating at 95 C (Z1); PCR cycling at 95 C (Z2); PCR cycling at 65 C (Z3) and DETECTR heating at 37 C (Z4). In some embodiments, a sample may be exposed to multiple heat zones within one chamber or channel, thus enabling constant sample fluid flow.

### Electrochemical DETECTR Reaction

The present disclosure provides various devices, systems, fluidic devices, and kits for rapid tests, which may quickly assess whether a target nucleic acid is present in a sample by using a programmable nuclease that can interact with functionalized surfaces of the fluidic systems to generate a detectable signal. For example, disclosed herein are particular microfluidic devices, lateral flow devices, sample preparation devices, and compositions (e.g., programmable nucleases, guide RNAs, reagents for in vitro transcription, amplification, reverse transcription, and reporters, or any combination thereof) for use in said devices that are particularly well suited to carry out a highly efficient, rapid, and accurate reactions for detecting the presence of a target nucleic acid (e.g., a DETECTR reaction). The systems and programmable nucleases disclosed herein can be used as a companion diagnostic with any of the diseases disclosed herein (e.g., RSV, sepsis, flu, COVID-19), or can be used in reagent kits, point-of-care diagnostics, or over-the-counter diagnostics. The systems may be used as a point of care diagnostic or as a lab test for detection of a target nucleic acid and, thereby, detection of a condition, for example, in a subject from which the sample was taken. The systems may be used in various sites or locations, such as in laboratories, in hospitals, in physician offices/laboratories (POLs), in clinics, at remotes sites, or at home. Sometimes, the present disclosure provides various devices, systems, fluidic devices, and kits for consumer genetic use or for over-the-counter use.

### Guide Nucleic Acids

Guide nucleic acids are compatible for use in the devices described herein (e.g., pneumatic valve devices, sliding valve devices, rotating valve devices, and lateral flow devices) and may be used in conjunction with compositions disclosed herein (e.g., programmable nucleases, reagents for in vitro transcription, reagents for amplification, reagents for reverse transcription, and reporters, or any combination thereof) to carry out highly efficient, rapid, and accurate reactions for detecting whether a target nucleic acid is present in a sample (e.g., DETECTR reactions). The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded target nucleic acid comprising a portion of a nucleic acid from a cancer gene or gene associated with a genetic disorder as described herein. The guide nucleic acid is complementary to the target nucleic acid. Often the guide nucleic acid binds specifically to the target nucleic acid. The target nucleic acid may be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid may comprise a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30 nt to about 40 nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of HPV 16 or HPV 18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling may comprise gaps between the tiled guide nucleic acids along the target nucleic acid. In some instances, the tiling of the guide nucleic acids is non-sequential. Often, a method for detecting a target nucleic acid may comprise contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that may be caused by multiple organisms. Exemplary guide nucleic acids are listed in Table 2. In Table 2, references to a “Cas12 Variant” refer to a Cas12 variant having the sequence of SEQ ID NO: 17, and references to “CasM.26” refer to a Cas13 variant having the sequence of SEQ ID NO: 21.

TABLE 2
Exemplary guide nucleic acids

| | | SEQ | |
|---|---|---|---|
| | | ID | |
| Name | Description | NO: | Sequence |
| R002 | CasM.26 control crRNA | 130 | GGCCACCCCAAAAAUGAAGGGGACUAAA |
| | | | ACACAAACAUGAUCUGGGUCAUC |
| R6052 | CasM.26 crRNA targeting MS2 | 131 | GCCACCCCAAAAAUGAAGGGGACUAAAA |
| | | | CACGGAGUUUGCUGCGAUUGCU |
| R439 | RNA target sequence for R002 | 132 | GCCAACCGCGAGAAGAUGACCCAGAUCA |
| | | | UGUUUGAGACCUUCAACACCCC |
| | CasM.26 crRNA targeting | 133 | GCCACCCCAAAAAUGAAGGGGACUAAAA |
| | MERS-CoV | | CAGCGACACUAAAACAUGAUGU |
| | CasM.26 crRNA targeting | 134 | GCCACCCCAAAAAUGAAGGGGACUAAAA |
| | MERS-CoV | | CAGGACAAAGGGUAACAUAGUU |
| | CasM.26 crRNA targeting | 135 | GCCACCCCAAAAAUGAAGGGGACUAAAA |
| | MERS-CoV | | CAGCAUUUACAAUAGAACCCCG |
| | CasM.26 crRNA targeting | 136 | GCCACCCCAAAAAUGAAGGGGACUAAAA |
| | human metapneumovirus | | CAGCUCCAUGCAAAUAUGAAGU |
| | CasM.26 crRNA targeting | 137 | GCCACCCCAAAAAUGAAGGGGACUAAAA |
| | human metapneumovirus | | CAACUCUGUAUGCUGCAUCACA |
| | CasM.26 crRNA targeting | 138 | GCCACCCCAAAAAUGAAGGGGACUAAAA |
| | human metapneumovirus | | CAUAACAUGGAAAGAUGUGAUG |
| | CasM.26 crRNA targeting | 139 | GCCACCCCAAAAAUGAAGGGGACUAAAA |
| | human parainfluenza 1 | | CAAUCUGGCUACUGAUUGCAAC |
| | CasM.26 crRNA targeting | 140 | GCCACCCCAAAAAUGAAGGGGACUAAAA |
| | human parainfluenza 1 | | CACAUGACAGUAUCCUCCGUGA |
| | CasM.26 crRNA targeting | 141 | GCCACCCCAAAAAUGAAGGGGACUAAAA |
| | human parainfluenza 1 | | CACAUCUGGCUACUGAUUGCAA |
| | CasM.26 crRNA targeting | 142 | GCCACCCCAAAAAUGAAGGGGACUAAAA |
| | human parainfluenza 2 | | CAAGCAGUCCUCACGCUAUUUU |
| | CasM.26 crRNA targeting | 143 | GCCACCCCAAAAAUGAAGGGGACUAAAA |
| | human parainfluenza 2 | | CAGGGAUAAUACAACAAUCUGC |
| | CasM.26 crRNA targeting | 144 | GCCACCCCAAAAAUGAAGGGGACUAAAA |
| | human parainfluenza 2 | | CACAAGUCAUGAUGGGUGCAGA |
| | CasM.26 crRNA targeting | 145 | GCCACCCCAAAAAUGAAGGGGACUAAAA |
| | human parainfluenza 4 | | CAAUGAAUCUAGGAACGGUACC |
| | CasM.26 crRNA targeting | 146 | GCCACCCCAAAAAUGAAGGGGACUAAAA |
| | human parainfluenza 4 | | CAAAUAGUACUUUUCGACGUGA |
| | CasM.26 crRNA targeting | 147 | GCCACCCCAAAAAUGAAGGGGACUAAAA |
| | human parainfluenza 4 | | CAAGAAUGUGGGGUCAUCCCAC |
| | CasM.26 crRNA targeting | 148 | GCCACCCCAAAAAUGAAGGGGACUAAAA |
| | hCoV-OC43 | | CAAGUAGCGCUAACUAGGGCUC |
| | CasM.26 crRNA targeting | 149 | GCCACCCCAAAAAUGAAGGGGACUAAAA |
| | hCoV-OC43 | | CAACCGCUUGUUAUAGCGGCAA |
| | CasM.26 crRNA targeting | 150 | GCCACCCCAAAAAUGAAGGGGACUAAAA |
| | hCoV-OC43 | | CAUGGCUGAACAGUGUAACCGU |
| | CasM.26 crRNA targeting | 151 | GCCACCCCAAAAAUGAAGGGGACUAAAA |
| | human parainfluenza 3 | | CAAUUGGUGAUCCAUCAGUUGC |
| | CasM.26 crRNA targeting | 152 | GCCACCCCAAAAAUGAAGGGGACUAAAA |
| | human parainfluenza 3 | | CAAAUUCAUACUCAGCACCGAC |
| | CasM.26 crRNA targeting | 153 | GCCACCCCAAAAAUGAAGGGGACUAAAA |
| | human parainfluenza 3 | | CAAACUUAGGAGCAAAGCGUGC |
| | CasM.26 crRNA targeting | 154 | GCCACCCCAAAAAUGAAGGGGACUAAAA |
| | RSV-A | | CAUUGCGUACAGUAGUGGAACU |
| | CasM.26 crRNA targeting | 155 | GCCACCCCAAAAAUGAAGGGGACUAAAA |
| | RSV-A | | CAAUCCAUUGGACCUCUCAAGA |
| | CasM.26 crRNA targeting | 156 | GCCACCCCAAAAAUGAAGGGGACUAAAA |
| | RSV-A | | CAAUAUGUCACGAAGGAAUCCU |
| | CasM.26 crRNA targeting | 157 | GCCACCCCAAAAAUGAAGGGGACUAAAA |
| | RSV-B | | CAAUAUUAGGAAUGCUCCAUAC |
| | CasM.26 crRNA targeting | 158 | GCCACCCCAAAAAUGAAGGGGACUAAAA |
| | RSV-B | | CAUAGACUGGGUAUAUGCAUCC |
| | CasM.26 crRNA targeting | 159 | GCCACCCCAAAAAUGAAGGGGACUAAAA |
| | RSV-B | | CAAAAGUACAGUCCAAUCGAGU |
| | CasM.26 crRNA targeting | 160 | GCCACCCCAAAAAUGAAGGGGACUAAAA |
| | hCoV-229E | | CAUACUCGUACGUAGAAAACCC |
| | CasM.26 crRNA targeting | 161 | GCCACCCCAAAAAUGAAGGGGACUAAAA |
| | hCoV-229E | | CAUACGGUUUAACGGUGGUUUG |
| | CasM.26 crRNA targeting | 162 | GCCACCCCAAAAAUGAAGGGGACUAAAA |
| | hCoV-229E | | CAGCCAUAGCACGUUUAUCAUA |
| | CasM.26 crRNA targeting | 163 | GCCACCCCAAAAAUGAAGGGGACUAAAA |
| | hCoV-HKU1 | | CACGUUGCGGACCUUUAAUCAU |
| | CasM.26 crRNA targeting | 164 | GCCACCCCAAAAAUGAAGGGGACUAAAA |
| | hCoV-HKU1 | | CAUACCGGACUUUACGGCUGUU |
| | CasM.26 crRNA targeting | 165 | GCCACCCCAAAAAUGAAGGGGACUAAAA |
| | hCoV-HKU1 | | CACGUCGUCAUCUAUACGCUGA |
| | CasM.26 crRNA targeting | 166 | GCCACCCCAAAAAUGAAGGGGACUAAAA |
| | hCoV-NL63 | | CACGUCUAAAACGUUUGGCACA |
| | CasM.26 crRNA targeting | 167 | GCCACCCCAAAAAUGAAGGGGACUAAAA |
| | hCoV-NL63 | | CAAGAACCCCUUGCUCGUUUAA |
| | CasM.26 crRNA targeting | 168 | GCCACCCCAAAAAUGAAGGGGACUAAAA |
| | hCoV-NL63 | | CAGAACACGCUUCCAACGAGGU |
| | CasM.26 crRNA targeting | 169 | GCCACCCCAAAAAUGAAGGGGACUAAAA |
| | Gammacoronavirus | | CACCCCGUACCCGUUUAAAUAA |
| | CasM.26 crRNA targeting | 170 | GCCACCCCAAAAAUGAAGGGGACUAAAA |
| | Gammacoronavirus | | CACCCGUACCCGUUUAAAUAAU |
| | CasM.26 crRNA targeting | 171 | GCCACCCCAAAAAUGAAGGGGACUAAAA |
| | Gammacoronavirus | | CAGUACCCGUUUAAAUAAUUCU |
| | CasM.26 crRNA targeting | 172 | GCCACCCCAAAAAUGAAGGGGACUAAAA |
| | Deltacoronavirus | | CAACAUUACAAUUCCAAAACAU |
| | CasM.26 crRNA targeting | 173 | GCCACCCCAAAAAUGAAGGGGACUAAAA |
| | Deltacoronavirus | | CAUCAACACUACAAAUGUAGUU |
| | CasM.26 crRNA targeting | 174 | GCCACCCCAAAAAUGAAGGGGACUAAAA |
| | Deltacoronavirus | | CAGCUUCACCAUCUUGCAUAUA |
| | CasM.26 crRNA targeting | 175 | GCCACCCCAAAAAUGAAGGGGACUAAAA |
| | Alphacoronavirus | | CAUCUAGUCGAGCUGCACUAGA |
| | CasM.26 crRNA targeting | 176 | GCCACCCCAAAAAUGAAGGGGACUAAAA |
| | Alphacoronavirus | | CAUCUAGUCGAGCUCCACUAGA |
| | CasM.26 crRNA targeting | 177 | GCCACCCCAAAAAUGAAGGGGACUAAAA |
| | Alphacoronavirus | | CAUCUAGUCGAGCGGCACUAGA |
| | CasM.26 crRNA targeting | 178 | GCCACCCCAAAAAUGAAGGGGACUAAAA |
| | Rhinovirus C | | CAACACGGACACCCAAAGUAGU |
| | CasM.26 crRNA targeting | 179 | GCCACCCCAAAAAUGAAGGGGACUAAAA |
| | Rhinovirus C | | CAACACGGUUACCCAAAGUAGU |
| | CasM.26 crRNA targeting | 180 | GCCACCCCAAAAAUGAAGGGGACUAAAA |
| | Rhinovirus C | | CACGGCCCCUGAAUGCGGCUAA |
| | CasM.26 crRNA targeting | 181 | GCCACCCCAAAAAUGAAGGGGACUAAAA |
| | Rhinovirus C | | CACGGCCCCUGAAUGUGGCUAA |
| | CasM.26 crRNA targeting | 182 | GCCACCCCAAAAAUGAAGGGGACUAAAA |
| | Rhinovirus C | | CACGGUCCCUGAAUGUGGCUAA |
| | CasM.26 crRNA targeting | 183 | GCCACCCCAAAAAUGAAGGGGACUAAAA |
| | Betacoronavirus | | CAUUCUUAGCACUAAUAGCAUA |
| | CasM.26 crRNA targeting | 184 | GCCACCCCAAAAAUGAAGGGGACUAAAA |
| | Betacoronavirus | | CAUUUUUAGCACUAAUGGCAUA |
| | CasM.26 crRNA targeting | 185 | GCCACCCCAAAAAUGAAGGGGACUAAAA |
| | Betacoronavirus | | CAUUCUUUGCACUAAUGGCAUA |
| | CasM.26 crRNA targeting | 186 | GCCACCCCAAAAAUGAAGGGGACUAAAA |
| | Betacoronavirus | | CAUUCUUAGCACUGAUAGCAUA |
| R504 | CasM.26 crRNA targeting | 187 | GCCACCCCAAAAAUGAAGGGGACUAAAA |
| | Influenza A | | CAAGUGAGCGAGGACUGCAGCG |
| R474 | CasM.26 crRNA targeting | 188 | GCCACCCCAAAAAUGAAGGGGACUAAAA |
| | Influenza A | | CACUUCUAACCGAGGUCGAAAC |
| R521 | CasM.26 crRNA targeting | 189 | GCCACCCCAAAAAUGAAGGGGACUAAAA |
| | Influenza A | | CAACAGCCAGACAGCGACCAAA |
| R534 | CasM.26 crRNA targeting | 190 | GCCACCCCAAAAAUGAAGGGGACUAAAA |
| | Influenza B | | CAGAAAAUGUUUCAAAUAUCAG |
| R567 | CasM.26 crRNA targeting | 191 | GCCACCCCAAAAAUGAAGGGGACUAAAA |
| | Influenza B | | CAACAUUGUAUGAAAUGAAGGA |
| R545 | CasM.26 crRNA targeting | 192 | GCCACCCCAAAAAUGAAGGGGACUAAAA |
| | Influenza B | | CAUGGGAGGGACACAGCAGAGG |
| | CasM.26 crRNA targeting | 193 | GCCACCCCAAAAAUGAAGGGGACUAAAA |
| | SARS-CoV-2 | | CAGUCUGUGCCAGCAUGAACUC |
| | CasM.26 crRNA targeting | 194 | GCCACCCCAAAAAUGAAGGGGACUAAAA |
| | SARS-CoV-2 | | CAGACUUUAGAUCGGCGCCGUA |
| | CasM.26 crRNA targeting | 195 | GCCACCCCAAAAAUGAAGGGGACUAAAA |
| | SARS-CoV-2 | | CAUAGACAACAGGUGCGCUCAG |
| | CasM.26 crRNA targeting | 196 | GCCACCCCAAAAAUGAAGGGGACUAAAA |
| | SARS-CoV-1 | | CAUAUCAACGGGCGUCUCGAGU |
| | CasM.26 crRNA targeting | 197 | GCCACCCCAAAAAUGAAGGGGACUAAAA |
| | SARS-CoV-1 | | CAUGCAUCAAGUGUGCGCGCAA |
| | CasM.26 crRNA targeting | 198 | GCCACCCCAAAAAUGAAGGGGACUAAAA |
| | SARS-CoV-1 | | CAGAGUGAUCGCAACUUUGCGC |
| | CasM.26 crRNA targeting | 199 | GCCACCCCAAAAAUGAAGGGGACUAAAA |
| | Sarbecovirus subgenus | | CAAGUACGCACACAAUCGAAGC |
| | CasM.26 crRNA targeting | 200 | GCCACCCCAAAAAUGAAGGGGACUAAAA |
| | Sarbecovirus subgenus | | CAACAAUCGAAGCGCAGUAAGG |
| | CasM.26 crRNA targeting | 201 | GCCACCCCAAAAAUGAAGGGGACUAAAA |
| | Sarbecovirus subgenus | | CAGCAGUACGCACACAAUCGAA |
| | CasM.26 crRNA targeting | 202 | GCCACCCCAAAAAUGAAGGGGACUAAAA |
| | SARS-related viruses | | CAUUCGGUCACACCCGGACGAA |
| | CasM.26 crRNA targeting | 203 | GCCACCCCAAAAAUGAAGGGGACUAAAA |
| | SARS-related viruses | | CACAACGGUAAUAGUACCGUUG |
| | CasM.26 crRNA targeting | 204 | GCCACCCCAAAAAUGAAGGGGACUAAAA |
| | SARS-related viruses | | CAUACCUUUCGGUCACACCCGG |
| R6052 | CasM.26 crRNA targeting MS2 | 131 | GCCACCCCAAAAAUGAAGGGGACUAAAA |
| | | | CACGGAGUUUGCUGCGAUUGCU |
| R6042 | CasM.26 crRNA targeting MS2 | 205 | GCCACCCCAAAAAUGAAGGGGACUAAAA |
| | | | CAGGAGACCUUGCAUUGCCUUA |
| | CasM.26 crRNA targeting | 206 | GCCACCCCAAAAAUGAAGGGGACUAAAA |
| | Influenza B-Victoria V1 | | CAAGAUCAAUAGUCGCUUCAAA |
| | CasM.26 crRNA targeting | 207 | GCCACCCCAAAAAUGAAGGGGACUAAAA |
| | Influenza B-Victoria V1 | | CAUUGUAACUUUCGAAUUCAGU |
| | CasM.26 crRNA targeting | 208 | GCCACCCCAAAAAUGAAGGGGACUAAAA |
| | Influenza B-Victoria V1 | | CACCUACUUCGUUCCCCCCAGA |
| | CasM.26 crRNA targeting | 209 | GCCACCCCAAAAAUGAAGGGGACUAAAA |
| | Influenza B-Yamagata Y1 | | CAAAACCCGCCAACACUGCAUU |
| | CasM.26 crRNA targeting | 210 | GCCACCCCAAAAAUGAAGGGGACUAAAA |
| | Influenza B-Yamagata Y1 | | CAUUUUCACACUUCAUUGUUGA |
| | CasM.26 crRNA targeting | 211 | GCCACCCCAAAAAUGAAGGGGACUAAAA |
| | Influenza B-Yamagata Y1 | | CAGGAUCUUCACUAAAUUCUGC |
| | CasM.26 crRNA targeting | 212 | GCCACCCCAAAAAUGAAGGGGACUAAAA |
| | Influenza A H1 | | CAGCACACAGAAUGCCAUUAAC |
| | CasM.26 crRNA targeting | 213 | GCCACCCCAAAAAUGAAGGGGACUAAAA |
| | Influenza A H1 | | CAACAUUUACAACCGCAAAUGC |
| | CasM.26 crRNA targeting | 214 | GCCACCCCAAAAAUGAAGGGGACUAAAA |
| | Influenza A H1 | | CAUACCCAGGAGAUUUCAUCAA |
| | CasM.26 crRNA targeting | 215 | GCCACCCCAAAAAUGAAGGGGACUAAAA |
| | Influenza A H1 | | CAAAUGAAAGAACUUUGGACUA |
| | CasM.26 crRNA targeting | 216 | GCCACCCCAAAAAUGAAGGGGACUAAAA |
| | Influenza A H1 | | CAAUGAAGGCAAUACUAGUAGU |
| | CasM.26 crRNA targeting | 217 | GCCACCCCAAAAAUGAAGGGGACUAAAA |
| | Influenza A H1 | | CAUCAUCAUUUGAAAGGUUUGA |
| | CasM.26 crRNA targeting | 218 | GCCACCCCAAAAAUGAAGGGGACUAAAA |
| | Influenza A H2 | | CAAGACAAAGAAUCCACUCAAA |
| | CasM.26 crRNA targeting | 219 | GCCACCCCAAAAAUGAAGGGGACUAAAA |
| | Influenza A H2 | | CAGCUAUCAUGAUUGCCAGUGA |
| | CasM.26 crRNA targeting | 220 | GCCACCCCAAAAAUGAAGGGGACUAAAA |
| | Influenza A H2 | | CACAGACAAAGAAUCCACUCAA |
| | CasM.26 crRNA targeting | 221 | GCCACCCCAAAAAUGAAGGGGACUAAAA |
| | Influenza A H3 | | CACAUGAUAUGGCAAAGGAAAU |
| | CasM.26 crRNA targeting | 222 | GCCACCCCAAAAAUGAAGGGGACUAAAA |
| | Influenza A H3 | | CAUGGAUUUCCUUUGCCAUAUC |
| | CasM.26 crRNA targeting | 223 | GCCACCCCAAAAAUGAAGGGGACUAAAA |
| | Influenza A H3 | | CAUCCCAACCAUUUUCUAUGAA |
| | CasM.26 crRNA targeting | 224 | GCCACCCCAAAAAUGAAGGGGACUAAAA |
| | Influenza A H4 | | CAUGUAGAACCAGGAGAUCUCA |
| | CasM.26 crRNA targeting | 225 | GCCACCCCAAAAAUGAAGGGGACUAAAA |
| | Influenza A H4 | | CACAACAACUGCAUCGAAAGCA |
| | CasM.26 crRNA targeting | 226 | GCCACCCCAAAAAUGAAGGGGACUAAAA |
| | Influenza A H4 | | CAGGCAACCAGAGGUCUGUUUG |
| | CasM.26 crRNA targeting | 227 | GCCACCCCAAAAAUGAAGGGGACUAAAA |
| | Influenza A H4 | | CAAUAGUUUUCAAUACCAUAGG |
| | CasM.26 crRNA targeting | 228 | GCCACCCCAAAAAUGAAGGGGACUAAAA |
| | Influenza A H4 | | CAUAAAUCUGAUGGGAAUGCAU |
| | CasM.26 crRNA targeting | 229 | GCCACCCCAAAAAUGAAGGGGACUAAAA |
| | Influenza A H4 | | CAUGCUCUGGGAAGCCCGGGUU |
| | CasM.26 crRNA targeting | 230 | GCCACCCCAAAAAUGAAGGGGACUAAAA |
| | Influenza A H5 | | CAGAAGACGGAUUCCUAGAUGU |
| | CasM.26 crRNA targeting | 231 | GCCACCCCAAAAAUGAAGGGGACUAAAA |
| | Influenza A H5 | | CAUCAGAAGAAGCAAGAUUAAA |
| | CasM.26 crRNA targeting | 230 | GCCACCCCAAAAAUGAAGGGGACUAAAA |
| | Influenza A H5 | | CAGAAGACGGAUUCCUAGAUGU |
| | CasM.26 crRNA targeting | 232 | GCCACCCCAAAAAUGAAGGGGACUAAAA |
| | Influenza A H5 | | CACAGUAUUCAGAAGAAGCAAG |
| | CasM.26 crRNA targeting | 233 | GCCACCCCAAAAAUGAAGGGGACUAAAA |
| | Influenza A H5 | | CAGACACACAACGGGAAGCUCU |
| | CasM.26 crRNA targeting | 234 | GCCACCCCAAAAAUGAAGGGGACUAAAA |
| | Influenza A H5 | | CAAGACAUACUGGAAAAGACAC |
| | CasM.26 crRNA targeting | 235 | GCCACCCCAAAAAUGAAGGGGACUAAAA |
| | Influenza A H6 | | CAGGUCUUUGGAUGUGUUCAAA |
| | CasM.26 crRNA targeting | 236 | GCCACCCCAAAAAUGAAGGGGACUAAAA |
| | Influenza A H6 | | CAUGGAUGUGUUCAAAUGGUUC |
| | CasM.26 crRNA targeting | 237 | GCCACCCCAAAAAUGAAGGGGACUAAAA |
| | Influenza A H6 | | CACUUUGGAUGUGUUCAAAUGG |
| | CasM.26 crRNA targeting | 238 | GCCACCCCAAAAAUGAAGGGGACUAAAA |
| | Influenza A H6 | | CAGUUCUUCUUGAAAACGAAAG |
| | CasM.26 crRNA targeting | 239 | GCCACCCCAAAAAUGAAGGGGACUAAAA |
| | Influenza A H6 | | CAAGUACGGUAUCGAGCAGUCU |
| | CasM.26 crRNA targeting | 240 | GCCACCCCAAAAAUGAAGGGGACUAAAA |
| | Influenza A H6 | | CACUUGGUGAUCAAAGCUGGUC |
| | CasM.26 crRNA targeting | 241 | GCCACCCCAAAAAUGAAGGGGACUAAAA |
| | Influenza A H7 | | CAAUAUACAAAUAGUGCACCGC |
| | CasM.26 crRNA targeting | 242 | GCCACCCCAAAAAUGAAGGGGACUAAAA |
| | Influenza A H7 | | CAUAUACAAAUAGUGCACCGCA |
| | CasM.26 crRNA targeting | 243 | GCCACCCCAAAAAUGAAGGGGACUAAAA |
| | Influenza A H7 | | CAUGCGGUGCACUAUUUGUAUA |
| | CasM.26 crRNA targeting | 244 | GCCACCCCAAAAAUGAAGGGGACUAAAA |
| | Influenza A H8 | | CAGAACAUGCAUCUACAAGAUC |
| | CasM.26 crRNA targeting | 245 | GCCACCCCAAAAAUGAAGGGGACUAAAA |
| | Influenza A H8 | | CAAACAUGCAUCUACAAGAUCC |
| | CasM.26 crRNA targeting | 246 | GCCACCCCAAAAAUGAAGGGGACUAAAA |
| | Influenza A H8 | | CAAUCUUGUAGAUGCAUGUUCU |
| | CasM.26 crRNA targeting | 247 | GCCACCCCAAAAAUGAAGGGGACUAAAA |
| | Influenza A H9 | | CAAAGGCAGCAAACCCCAUUGC |
| | CasM.26 crRNA targeting | 248 | GCCACCCCAAAAAUGAAGGGGACUAAAA |
| | Influenza A H9 | | CAAGGCAGCAAACCCCAUUGCA |
| | CasM.26 crRNA targeting | 249 | GCCACCCCAAAAAUGAAGGGGACUAAAA |
| | Influenza A H9 | | CAGGCAGCAAACCCCAUUGCAA |
| | CasM.26 crRNA targeting | 250 | GCCACCCCAAAAAUGAAGGGGACUAAAA |
| | Influenza A H10 | | CAGGUUUAGCUUCGGGGCAUCA |
| | CasM.26 crRNA targeting | 251 | GCCACCCCAAAAAUGAAGGGGACUAAAA |
| | Influenza A H10 | | CAUGUGGGGAAUUCAUCACCCU |
| | CasM.26 crRNA targeting | 252 | GCCACCCCAAAAAUGAAGGGGACUAAAA |
| | Influenza A H10 | | CAUCACCUUCUCACACAAUGGG |
| | CasM.26 crRNA targeting | 253 | GCCACCCCAAAAAUGAAGGGGACUAAAA |
| | Influenza A H10 | | CAUGGAAACAUGCGAUGCACAA |
| | CasM.26 crRNA targeting | 254 | GCCACCCCAAAAAUGAAGGGGACUAAAA |
| | Influenza A H10 | | CACUGAGCUAUUGGUAGCAAUG |
| | CasM.26 crRNA targeting | 255 | GCCACCCCAAAAAUGAAGGGGACUAAAA |
| | Influenza A H10 | | CAUCUUGAUAAAAUCUGCCUAG |
| | CasM.26 crRNA targeting | 256 | GCCACCCCAAAAAUGAAGGGGACUAAAA |
| | Influenza A H11 | | CAACAGCUGCAUUGCAAGCAGU |
| | CasM.26 crRNA targeting | 257 | GCCACCCCAAAAAUGAAGGGGACUAAAA |
| | Influenza A H11 | | CAUGCAAAUGGUACAUCUACAU |
| | CasM.26 crRNA targeting | 258 | GCCACCCCAAAAAUGAAGGGGACUAAAA |
| | Influenza A H11 | | CAACUUGCAAUGCAGCUGUAAA |
| | CasM.26 crRNA targeting | 259 | GCCACCCCAAAAAUGAAGGGGACUAAAA |
| | Influenza A H12 | | CAAUGAAACCCCCAAUAAUCAU |
| | CasM.26 crRNA targeting | 260 | GCCACCCCAAAAAUGAAGGGGACUAAAA |
| | Influenza A H12 | | CAAAUGAAACCCCCAAUAAUCA |
| | CasM.26 crRNA targeting | 261 | GCCACCCCAAAAAUGAAGGGGACUAAAA |
| | Influenza A H12 | | CAUAAGCUUGAGGCAACACUGC |
| | CasM.26 crRNA targeting | 262 | GCCACCCCAAAAAUGAAGGGGACUAAAA |
| | Influenza A H13 | | CAACAACACUGCUUGCAAUGCA |
| | CasM.26 crRNA targeting | 263 | GCCACCCCAAAAAUGAAGGGGACUAAAA |
| | Influenza A H13 | | CACAACACUGCUUGCAAUGCAG |
| | CasM.26 crRNA targeting | 264 | GCCACCCCAAAAAUGAAGGGGACUAAAA |
| | Influenza A H13 | | CAUACAACACUGCUUGCAAUGC |
| | CasM.26 crRNA targeting | 265 | GCCACCCCAAAAAUGAAGGGGACUAAAA |
| | Influenza A H14 | | CAACAAAGCAUGACAUGGAGAA |
| | CasM.26 crRNA targeting | 266 | GCCACCCCAAAAAUGAAGGGGACUAAAA |
| | Influenza A H14 | | CAUAGAGAAGACAAAUGAAAAA |
| | CasM.26 crRNA targeting | 267 | GCCACCCCAAAAAUGAAGGGGACUAAAA |
| | Influenza A H14 | | CAACCCAUUCGAUGUUCCAGAU |
| | CasM.26 crRNA targeting | 268 | GCCACCCCAAAAAUGAAGGGGACUAAAA |
| | Influenza A H15 | | CAAAGGGGCAUUAGAGCGGAGA |
| | CasM.26 crRNA targeting | 269 | GCCACCCCAAAAAUGAAGGGGACUAAAA |
| | Influenza A H15 | | CAACCAGGUGUUCCCACAACUA |
| | CasM.26 crRNA targeting | 270 | GCCACCCCAAAAAUGAAGGGGACUAAAA |
| | Influenza A H15 | | CAUACAAAAUAGGAUAAUGAUC |
| | CasM.26 crRNA targeting | 271 | GCCACCCCAAAAAUGAAGGGGACUAAAA |
| | Influenza A H16 | | CAGAGUGGAGUGAGGAUGGCCA |
| | CasM.26 crRNA targeting | 272 | GCCACCCCAAAAAUGAAGGGGACUAAAA |
| | Influenza A H16 | | CAGGUGAUUUGGGGAAUCCACC |
| | CasM.26 crRNA targeting | 273 | GCCACCCCAAAAAUGAAGGGGACUAAAA |
| | Influenza A H16 | | CAUGGAGUAAACUCUUUUAGCA |
| | CasM.26 crRNA targeting | 274 | GCCACCCCAAAAAUGAAGGGGACUAAAA |
| | Influenza A H16 | | CACAAACGCUAUGAGCUCGAGA |
| | CasM.26 crRNA targeting | 275 | GCCACCCCAAAAAUGAAGGGGACUAAAA |
| | Influenza A H16 | | CAGCACGAUGCAAAUGUCAGAA |
| | CasM.26 crRNA targeting | 276 | GCCACCCCAAAAAUGAAGGGGACUAAAA |
| | Influenza A H16 | | CAGAUGAACGGAAAUUAUGAUU |
| | CasM.26 crRNA targeting | 277 | GCCACCCCAAAAAUGAAGGGGACUAAAA |
| | Influenza A N1 | | CAUGGUCUUGGCCAGACGGUGC |
| | CasM.26 crRNA targeting | 278 | GCCACCCCAAAAAUGAAGGGGACUAAAA |
| | Influenza A N1 | | CAUCCAAGGGGGAUGUGUUUGU |
| | CasM.26 crRNA targeting | 279 | GCCACCCCAAAAAUGAAGGGGACUAAAA |
| | Influenza A N1 | | CAAGAACACAAGAGUCUGAAUG |
| | CasM.26 crRNA targeting | 280 | GCCACCCCAAAAAUGAAGGGGACUAAAA |
| | Influenza A N1 | | CAGUUUGGAUAGGGAGAACUAA |
| | CasM.26 crRNA targeting | 281 | GCCACCCCAAAAAUGAAGGGGACUAAAA |
| | Influenza A N1 | | CAUUUGAGUCAGUCGCUUGGUC |
| | CasM.26 crRNA targeting | 282 | GCCACCCCAAAAAUGAAGGGGACUAAAA |
| | Influenza A N1 | | CAUCUAGUGAAAUCACAUGUGU |
| | CasM.26 crRNA targeting | 283 | GCCACCCCAAAAAUGAAGGGGACUAAAA |
| | Influenza A N2 | | CAUUAUGGAACAGGCUCAUGGC |
| | CasM.26 crRNA targeting | 284 | GCCACCCCAAAAAUGAAGGGGACUAAAA |
| | Influenza A N2 | | CACUCCAUCAGGCCAUGAGCCU |
| | CasM.26 crRNA targeting | 285 | GCCACCCCAAAAAUGAAGGGGACUAAAA |
| | Influenza A N2 | | CAUGCCCCAUCAGGCCAUGAGC |
| | CasM.26 crRNA targeting | 286 | GCCACCCCAAAAAUGAAGGGGACUAAAA |
| | Influenza A N3 | | CAAUCCUUUGAUUUGGAUUCAU |
| | CasM.26 crRNA targeting | 287 | GCCACCCCAAAAAUGAAGGGGACUAAAA |
| | Influenza A N3 | | CAUUAUCUUCUGAUUUGGAUUC |
| | CasM.26 crRNA targeting | 288 | GCCACCCCAAAAAUGAAGGGGACUAAAA |
| | Influenza A N3 | | CAUAUCCUUUGAUUCGGAUUCA |
| | CasM.26 crRNA targeting | 289 | GCCACCCCAAAAAUGAAGGGGACUAAAA |
| | Influenza A N4 | | CAGACCAACCUGUGGUAUCAGA |
| | CasM.26 crRNA targeting | 290 | GCCACCCCAAAAAUGAAGGGGACUAAAA |
| | Influenza A N4 | | CACUCUGUUGCCUUUUGACAUA |
| | CasM.26 crRNA targeting | 291 | GCCACCCCAAAAAUGAAGGGGACUAAAA |
| | Influenza A N4 | | CACCAACCUGUGGUAUCGGAAU |
| | CasM.26 crRNA targeting | 292 | GCCACCCCAAAAAUGAAGGGGACUAAAA |
| | Influenza A N5 | | CAUAAUAACAGACACUAUCAAG |
| | CasM.26 crRNA targeting | 293 | GCCACCCCAAAAAUGAAGGGGACUAAAA |
| | Influenza A N5 | | CAUGGAAUGCAGAACUUUCUUU |
| | CasM.26 crRNA targeting | 294 | GCCACCCCAAAAAUGAAGGGGACUAAAA |
| | Influenza A N5 | | CAGAACUCAAGAGUCUGAAUGU |
| | CasM.26 crRNA targeting | 295 | GCCACCCCAAAAAUGAAGGGGACUAAAA |
| | Influenza A N5 | | CAGCGGCUUUGAAAUGAUUUGG |
| | CasM.26 crRNA targeting | 296 | GCCACCCCAAAAAUGAAGGGGACUAAAA |
| | Influenza A N5 | | CAGUGUCUGGAUCGGGAGAACC |
| | CasM.26 crRNA targeting | 297 | GCCACCCCAAAAAUGAAGGGGACUAAAA |
| | Influenza A N5 | | CAUCGAAUUGGAUGCUCCUAAU |
| | CasM.26 crRNA targeting | 298 | GCCACCCCAAAAAUGAAGGGGACUAAAA |
| | Influenza A N6 | | CAAUGAUGACCCACACAAGCAA |
| | CasM.26 crRNA targeting | 299 | GCCACCCCAAAAAUGAAGGGGACUAAAA |
| | Influenza A N6 | | CAAAGAUAACAUGCAUUUCAGC |
| | CasM.26 crRNA targeting | 300 | GCCACCCCAAAAAUGAAGGGGACUAAAA |
| | Influenza A N6 | | CAAAUGACCCCACCAAUGGGAA |
| | CasM.26 crRNA targeting | 301 | GCCACCCCAAAAAUGAAGGGGACUAAAA |
| | Influenza A N6 | | CAGAAUGCAUAGGAUGGUCAAG |
| | CasM.26 crRNA targeting | 302 | GCCACCCCAAAAAUGAAGGGGACUAAAA |
| | Influenza A N6 | | CAAACAUUCCAAACAUGAAUGA |
| | CasM.26 crRNA targeting | 303 | GCCACCCCAAAAAUGAAGGGGACUAAAA |
| | Influenza A N6 | | CAAAUAACAAUGCAUCGGCAGU |
| | CasM.26 crRNA targeting | 304 | GCCACCCCAAAAAUGAAGGGGACUAAAA |
| | Influenza A N7 | | CAGUGAUUGAGAAUGAAUCCUA |
| | CasM.26 crRNA targeting | 305 | GCCACCCCAAAAAUGAAGGGGACUAAAA |
| | Influenza A N7 | | CAUGAAAGUGAACAAAUAAUAG |
| | CasM.26 crRNA targeting | 306 | GCCACCCCAAAAAUGAAGGGGACUAAAA |
| | Influenza A N7 | | CAUUCCUUCCCCGAUGGGGCAC |
| | CasM.26 crRNA targeting | 307 | GCCACCCCAAAAAUGAAGGGGACUAAAA |
| | Influenza A N7 | | CAUUGGUGGACGAGCAACAGUU |
| | CasM.26 crRNA targeting | 308 | GCCACCCCAAAAAUGAAGGGGACUAAAA |
| | Influenza A N7 | | CAAGGAAGUUUCAUUGACUAUU |
| | CasM.26 crRNA targeting | 309 | GCCACCCCAAAAAUGAAGGGGACUAAAA |
| | Influenza A N7 | | CAUACCACAAUAAAAACAUGGG |
| | CasM.26 crRNA targeting | 310 | GCCACCCCAAAAAUGAAGGGGACUAAAA |
| | Influenza A N8 | | CAGGCUCCAUUUCAUUAGGGUU |
| | CasM.26 crRNA targeting | 311 | GCCACCCCAAAAAUGAAGGGGACUAAAA |
| | Influenza A N8 | | CAUGGCACGAUGGAGCUAUUCU |
| | CasM.26 crRNA targeting | 312 | GCCACCCCAAAAAUGAAGGGGACUAAAA |
| | Influenza A N8 | | CACACGAUGGAGCUAUUCUUCC |
| | CasM.26 crRNA targeting | 313 | GCCACCCCAAAAAUGAAGGGGACUAAAA |
| | Influenza A N8 | | CAAUGAAUCCAAAUCAGAAAAU |
| | CasM.26 crRNA targeting | 314 | GCCACCCCAAAAAUGAAGGGGACUAAAA |
| | Influenza A N8 | | CAGAUGGAGCUAUUCUUCCUUU |
| | CasM.26 crRNA targeting | 315 | GCCACCCCAAAAAUGAAGGGGACUAAAA |
| | Influenza A N8 | | CAUGGUCAGGAUACAGUGGUUC |
| | CasM.26 crRNA targeting | 316 | GCCACCCCAAAAAUGAAGGGGACUAAAA |
| | Influenza A N9 | | CAUGGCCACUAUCAUCACCGCC |
| | CasM.26 crRNA targeting | 317 | GCCACCCCAAAAAUGAAGGGGACUAAAA |
| | Influenza A N9 | | CAUUGAACAUAGGACUGCAUCU |
| | CasM.26 crRNA targeting | 318 | GCCACCCCAAAAAUGAAGGGGACUAAAA |
| | Influenza A N9 | | CAAUGUUAAAAGUGCCAAAUGC |
| | CasM.26 crRNA targeting | 319 | GCCACCCCAAAAAUGAAGGGGACUAAAA |
| | Influenza A N9 | | CAAUCACCCAAAUAAGCAACAC |
| | CasM.26 crRNA targeting | 320 | GCCACCCCAAAAAUGAAGGGGACUAAAA |
| | Influenza A N9 | | CAACAGAAUUCCUGGGACAAUG |
| | CasM.26 crRNA targeting | 321 | GCCACCCCAAAAAUGAAGGGGACUAAAA |
| | Influenza A N9 | | CAACCAGCAAUAGUAUAGUAUC |
| | Cas12 Variant gRNA targeting | 322 | UAAUUUCUACUAAGUGUAGAUAGCAAUC |
| | Bordetella parapertussis | | CUGGCCACUAUUU |
| | Cas12 Variant gRNA targeting | 323 | UAAUUUCUACUAAGUGUAGAUUAGUUCC |
| | Bordetella parapertussis | | ACCUGAAGGCAUG |
| | Cas12 Variant gRNA targeting | 324 | UAAUUUCUACUAAGUGUAGAUGAUGCG |
| | Bordetella parapertussis | | GUGAGUAUGGCGAC |
| | Cas12 Variant gRNA targeting | 325 | UAAUUUCUACUAAGUGUAGAUGGGUAC |
| | Bordetella pertussis | | UUCAAUGGCCGCGA |
| | Cas12 Variant gRNA targeting | 326 | UAAUUUCUACUAAGUGUAGAUGCCUGAA |
| | Bordetella pertussis | | CCCUCCUAGCUGU |
| | Cas12 Variant gRNA targeting | 327 | UAAUUUCUACUAAGUGUAGAUCAUGAA |
| | Bordetella pertussis | | GAGCUUGGCUUCCA |
| | Cas12 Variant gRNA targeting | 328 | UAAUUUCUACUAAGUGUAGAUUACGACG |
| | Bordetella bronchoseptica | | UCUACGAAUGCGC |
| | Cas12 Variant gRNA targeting | 329 | UAAUUUCUACUAAGUGUAGAUGCGUUCG |
| | Bordetella bronchoseptica | | AUUACCUGGAAGG |
| | Cas12 Variant gRNA targeting | 330 | UAAUUUCUACUAAGUGUAGAUAUCGGCC |
| | Bordetella bronchoseptica | | AUGAAGGCCGCCU |
| | Cas12 Variant gRNA targeting | 331 | UAAUUUCUACUAAGUGUAGAUGCCACUG |
| | Bordetella holmesii | | CCAUCUCUUUCAU |
| | Cas12 Variant gRNA targeting | 332 | UAAUUUCUACUAAGUGUAGAUCGCCUGC |
| | Bordetella holmesii | | UUGGUAGGCCCAG |
| | Cas12 Variant gRNA targeting | 333 | UAAUUUCUACUAAGUGUAGAUCCUGAAG |
| | Bordetella holmesii | | CCUUCAGCGCGAA |
| | Cas12 Variant gRNA targeting | 334 | UAAUUUCUACUAAGUGUAGAUUAUCCUG |
| | Chlamydophila pneumoniae | | AGGUUUCAGUCUC |
| | Cas12 Variant gRNA targeting | 335 | UAAUUUCUACUAAGUGUAGAUGGAUGU |
| | Chlamydophila pneumoniae | | GUGAUGCUUGCUUU |
| | Cas12 Variant gRNA targeting | 336 | UAAUUUCUACUAAGUGUAGAUGCUCAGC |
| | Chlamydophila pneumoniae | | UGCCUAAGGAAUU |
| | Cas12 Variant gRNA targeting | 337 | UAAUUUCUACUAAGUGUAGAUGAUUUG |
| | Legionella pneumophila | | GGCGGCAAUGUGAA |
| | Cas12 Variant gRNA targeting | 338 | UAAUUUCUACUAAGUGUAGAUACGCAAC |
| | Legionella pneumophila | | ACCAGGAAGUGGA |
| | Cas12 Variant gRNA targeting | 339 | UAAUUUCUACUAAGUGUAGAUCCGGGAG |
| | Legionella pneumophila | | CCAGUUCCAAUUA |
| | Cas12 Variant gRNA targeting | 340 | UAAUUUCUACUAAGUGUAGAUAGCUCGA |
| | Mycoplasma pneumoniae | | UAAGGGCCGCGCG |
| | Cas12 Variant gRNA targeting | 341 | UAAUUUCUACUAAGUGUAGAUGUGGCA |
| | Mycoplasma pneumoniae | | GUUGGGUCAACCGG |
| | Cas12 Variant gRNA targeting | 342 | UAAUUUCUACUAAGUGUAGAUUGGUAA |
| | Mycoplasma pneumoniae | | AGGACCUUGCGCAU |
| | Cas12 Variant gRNA targeting | 343 | UAAUUUCUACUAAGUGUAGAUUGUAAU |
| | Human bocavirus | | GUUCUGGAAGCGGC |
| | Cas12 Variant gRNA targeting | 344 | UAAUUUCUACUAAGUGUAGAUGUGGGA |
| | Human bocavirus | | GGAGUGCUUAAUGC |
| | Cas12 Variant gRNA targeting | 345 | UAAUUUCUACUAAGUGUAGAUGCAUUCC |
| | Human bocavirus | | UCUUGCUUAGUCC |
| | Cas12 Variant gRNA targeting | 346 | UAAUUUCUACUAAGUGUAGAUCUCGUCA |
| | Human bocavirus | | GUUAUCUCGUCUG |
| | Cas12 Variant gRNA targeting | 347 | UAAUUUCUACUAAGUGUAGAUAGUAUG |
| | Human bocavirus | | GCUGAAUUCUGCCU |
| | Cas12 Variant gRNA targeting | 348 | UAAUUUCUACUAAGUGUAGAUCUGCAGG |
| | Human adenovirus Type A | | UGUUUGAAGAUCC |
| | Cas12 Variant gRNA targeting | 349 | UAAUUUCUACUAAGUGUAGAUUCCAGGC |
| | Human adenovirus Type A | | UCAAGAAUGGAGC |
| | Cas12 Variant gRNA targeting | 350 | UAAUUUCUACUAAGUGUAGAUUUAAAG |
| | Human adenovirus Type A | | ACGUGCUACCUGAG |
| | Cas12 Variant gRNA targeting | 351 | UAAUUUCUACUAAGUGUAGAUACUCCUC |
| | Human adenovirus Type A | | CUAUGCGCGAUGU |
| | Cas12 Variant gRNA targeting | 352 | UAAUUUCUACUAAGUGUAGAUCCAGGGU |
| | Human adenovirus Type A | | GAAGUAGGUGUCC |
| | Cas12 Variant gRNA targeting | 353 | UAAUUUCUACUAAGUGUAGAUGAACCUC |
| | Human adenovirus Type B | | UUCCAAGGAAGCU |
| | Cas12 Variant gRNA targeting | 354 | UAAUUUCUACUAAGUGUAGAUCAUCACA |
| | Human adenovirus Type B | | AGACAAGCCACAG |
| | Cas12 Variant gRNA targeting | 355 | UAAUUUCUACUAAGUGUAGAUAAGCUG |
| | Human adenovirus Type B | | AUGCCAGAGCAGCA |
| | Cas12 Variant gRNA targeting | 356 | UAAUUUCUACUAAGUGUAGAUUUGAUG |
| | Human adenovirus Type C | | GCUUGCAAUUGGCC |
| | Cas12 Variant gRNA targeting | 357 | UAAUUUCUACUAAGUGUAGAUUCCACCA |
| | Human adenovirus Type C | | GACCACGGAAGAG |
| | Cas12 Variant gRNA targeting | 358 | UAAUUUCUACUAAGUGUAGAUCUCCAGC |
| | Human adenovirus Type C | | GCGCUUACAAAGU |
| | Cas12 Variant gRNA targeting | 359 | UAAUUUCUACUAAGUGUAGAUCUAGGG |
| | Human adenovirus Type D | | GAAGAUGGAGGAGC |
| | Cas12 Variant gRNA targeting | 360 | UAAUUUCUACUAAGUGUAGAUCCUCCCA |
| | Human adenovirus Type D | | GCGAUUCAGAGGA |
| | Cas12 Variant gRNA targeting | 361 | UAAUUUCUACUAAGUGUAGAUUCACCGA |
| | Human adenovirus Type D | | GCGCGGACACAGA |
| | Cas12 Variant gRNA targeting | 362 | UAAUUUCUACUAAGUGUAGAUAUGAAG |
| | Human adenovirus Type E | | GCCAUGUACCCGGG |
| | Cas12 Variant gRNA targeting | 363 | UAAUUUCUACUAAGUGUAGAUUACAAGC |
| | Human adenovirus Type E | | GGCCACAGUGCUC |
| | Cas12 Variant gRNA targeting | 364 | UAAUUUCUACUAAGUGUAGAUUUGUCCG |
| | Human adenovirus Type E | | AGAGCACGUUGUA |
| | Cas12 Variant gRNA targeting | 365 | UAAUUUCUACUAAGUGUAGAUUCUCGUU |
| | Human adenovirus Type F | | GUAGACGACACUG |
| | Cas12 Variant gRNA targeting | 366 | UAAUUUCUACUAAGUGUAGAUUCUCUUA |
| | Human adenovirus Type F | | AUAGACGCCCCAC |
| | Cas12 Variant gRNA targeting | 367 | UAAUUUCUACUAAGUGUAGAUUCCUCCC |
| | Human adenovirus Type F | | CUUACAAACAGUA |
| | Cas12 Variant gRNA targeting | 368 | UAAUUUCUACUAAGUGUAGAUGCACUAG |
| | Human adenovirus Type F | | AUGCAGCGCUGCC |
| | Cas12 Variant gRNA targeting | 369 | UAAUUUCUACUAAGUGUAGAUACCACAG |
| | Human adenovirus Type F | | CUGGGUAUUUGGA |
| | Cas12 Variant gRNA targeting | 370 | UAAUUUCUACUAAGUGUAGAUAACCAUG |
| | Human adenovirus Type F | | AAGGUGUGCCUGC |
| | Cas12 Variant gRNA targeting | 371 | UAAUUUCUACUAAGUGUAGAUAAUACCC |
| | Human adenovirus Type G | | AGCGAAAACGCUG |
| | Cas12 Variant gRNA targeting | 372 | UAAUUUCUACUAAGUGUAGAUGCUCCAC |
| | Human adenovirus Type G | | GAUUAAAUCCUAC |
| | Cas12 Variant gRNA targeting | 373 | UAAUUUCUACUAAGUGUAGAUGGUCAAC |
| | Human adenovirus Type G | | AUGUAUCUGAGCC |
| | CasM.26 crRNA targeting | 374 | GCCACCCCAAAAAUGAAGGGGACUAAAA |
| | Bordetella holmesii | | CACCCCCGCUACCGGGCACGUU |
| | CasM.26 crRNA targeting | 375 | GCCACCCCAAAAAUGAAGGGGACUAAAA |
| | Bordetella holmesii | | CAGCUACCGGGCACGUUCCGAU |
| | CasM.26 crRNA targeting | 376 | GCCACCCCAAAAAUGAAGGGGACUAAAA |
| | Bordetella holmesii | | CAACACUCUAGCUCGGUAGUUA |
| | CasM.26 crRNA targeting | 377 | GCCACCCCAAAAAUGAAGGGGACUAAAA |
| | Bordetella holmesii | | CAACACACCCGCAGCUUCGGUU |
| | CasM.26 crRNA targeting | 378 | GCCACCCCAAAAAUGAAGGGGACUAAAA |
| | Bordetella holmesii | | CAUACAUAGUACACACCCGCAG |
| | CasM.26 crRNA targeting | 379 | GCCACCCCAAAAAUGAAGGGGACUAAAA |
| | Bordetella holmesii | | CACGUGUACAUAGUACACACCC |
| | CasM.26 crRNA targeting | 380 | GCCACCCCAAAAAUGAAGGGGACUAAAA |
| | Bordetella holmesii | | CAUCGCGCUGAAGGCUUCAGGU |
| | CasM.26 crRNA targeting | 381 | GCCACCCCAAAAAUGAAGGGGACUAAAA |
| | Bordetella holmesii | | CAAUCGUCUUGCGGUCCAGCAU |
| | CasM.26 crRNA targeting | 382 | GCCACCCCAAAAAUGAAGGGGACUAAAA |
| | Bordetella holmesii | | CAGAUCGUCUUGCGGUCCAGCA |
| | CasM.26 crRNA targeting | 383 | GCCACCCCAAAAAUGAAGGGGACUAAAA |
| | Bordetella pertussis | | CACUACCGCGCGCACUAAAAGU |
| | CasM.26 crRNA targeting | 384 | GCCACCCCAAAAAUGAAGGGGACUAAAA |
| | Bordetella pertussis | | CAGCGCACUAAAAGUGCACACC |
| | CasM.26 crRNA targeting | 385 | GCCACCCCAAAAAUGAAGGGGACUAAAA |
| | Bordetella pertussis | | CAGCGCGCACUAAAAGUGCACA |
| | CasM.26 crRNA targeting | 386 | GCCACCCCAAAAAUGAAGGGGACUAAAA |
| | Bordetella pertussis | | CACUACAACUGGCAGCGACCCC |
| | CasM.26 crRNA targeting | 387 | GCCACCCCAAAAAUGAAGGGGACUAAAA |
| | Bordetella pertussis | | CAACCACUACAACUGGCAGCGA |
| | CasM.26 crRNA targeting | 388 | GCCACCCCAAAAAUGAAGGGGACUAAAA |
| | Bordetella pertussis | | CAUACAACUGGCAGCGACCCCA |
| | CasM.26 crRNA targeting | 389 | GCCACCCCAAAAAUGAAGGGGACUAAAA |
| | Bordetella parapertussis | | CAUAUUCCGCUUUGCUCGGCCU |
| | CasM.26 crRNA targeting | 390 | GCCACCCCAAAAAUGAAGGGGACUAAAA |
| | Bordetella parapertussis | | CAAGCAACUUGCGAUCCAGCAU |
| | CasM.26 crRNA targeting | 391 | GCCACCCCAAAAAUGAAGGGGACUAAAA |
| | Bordetella parapertussis | | CAGGCUAUUCCGCUUUGCUCGG |
| | CasM.26 crRNA targeting | 392 | GCCACCCCAAAAAUGAAGGGGACUAAAA |
| | Bordetella bronchiosceptica | | CAGGUCAUCAGGGUGUUGGAUG |
| | CasM.26 crRNA targeting | 393 | GCCACCCCAAAAAUGAAGGGGACUAAAA |
| | Bordetella bronchiosceptica | | CAGAUGGCUCGCCGGGUAGGGC |
| | CasM.26 crRNA targeting | 394 | GCCACCCCAAAAAUGAAGGGGACUAAAA |
| | Bordetella bronchiosceptica | | CACCGGGUAGGGCGGCCAAUUG |
| | CasM.26 crRNA targeting | 395 | GCCACCCCAAAAAUGAAGGGGACUAAAA |
| | Bordetella bronchiosceptica | | CAGAUACCGACUCGGCGCUGCG |
| | CasM.26 crRNA targeting | 396 | GCCACCCCAAAAAUGAAGGGGACUAAAA |
| | Influenza A | | CAAUAAUAACAAUUGGCUCAAU |
| | CasM.26 crRNA targeting | 397 | GCCACCCCAAAAAUGAAGGGGACUAAAA |
| | Influenza A | | CAAUAACAAUUGGCUCAAUAUC |
| | CasM.26 crRNA targeting | 398 | GCCACCCCAAAAAUGAAGGGGACUAAAA |
| | Influenza A | | CAACAAUUGGCUCAAUAUCAUU |
| | CasM.26 crRNA targeting | 399 | GCCACCCCAAAAAUGAAGGGGACUAAAA |
| | chlamydia pneumoniae | | CACGCUCGUUGCGGGACUUAAC |
| | CasM.26 crRNA targeting | 400 | GCCACCCCAAAAAUGAAGGGGACUAAAA |
| | chlamydia pneumoniae | | CAUGGCGCGAUGAUUCUCGAUU |
| | CasM.26 crRNA targeting | 401 | GCCACCCCAAAAAUGAAGGGGACUAAAA |
| | chlamydia pneumoniae | | CAGGUCUGGGCUGUUCCCCUCU |
| | CasM.26 crRNA targeting | 402 | GCCACCCCAAAAAUGAAGGGGACUAAAA |
| | chlamydia pneumoniae | | CACGACUGGGUUGUAGCAAAAC |
| | CasM.26 crRNA targeting | 403 | GCCACCCCAAAAAUGAAGGGGACUAAAA |
| | legionella pneumophila | | CAGUAUCAGUGUUCCCGAAGGC |
| | CasM.26 crRNA targeting | 404 | GCCACCCCAAAAAUGAAGGGGACUAAAA |
| | legionella pneumophila | | CAAGGGUGGCUGCUUCUAAGCC |
| | CasM.26 crRNA targeting | 405 | GCCACCCCAAAAAUGAAGGGGACUAAAA |
| | legionella pneumophila | | CAAGGGGAAGCCUUUUUAACUG |
| | CasM.26 crRNA targeting | 406 | GCCACCCCAAAAAUGAAGGGGACUAAAA |
| | mycoplasma pneumoniae | | CAUAUAAGGUAGAUUGGAUACG |
| | CasM.26 crRNA targeting | 407 | GCCACCCCAAAAAUGAAGGGGACUAAAA |
| | mycoplasma pneumoniae | | CAGGUUUGGGAGAUUCUCACUC |
| | CasM.26 crRNA targeting | 408 | GCCACCCCAAAAAUGAAGGGGACUAAAA |
| | mycoplasma pneumoniae | | CACGGUUUGGGAGAUUCUCACU |
| | CasM.26 crRNA targeting | 409 | GCCACCCCAAAAAUGAAGGGGACUAAAA |
| | mycoplasma pneumoniae | | CAACCGGGUUGAGGUGGGAAAC |
| | CasM.26 crRNA targeting | 410 | GCCACCCCAAAAAUGAAGGGGACUAAAA |
| | mycoplasma pneumoniae | | CACCCGGUAUGGCGCCUCGUCC |
| | CasM.26 crRNA targeting | 411 | GCCACCCCAAAAAUGAAGGGGACUAAAA |
| | human 18S | | CAGGUGACGGGGAAUCAGGGUU |
| | CasM.26 crRNA targeting | 412 | GCCACCCCAAAAAUGAAGGGGACUAAAA |
| | human 18S | | CAUGUCCGGGCCGGGUGAGGUU |
| | CasM.26 crRNA targeting | 413 | GCCACCCCAAAAAUGAAGGGGACUAAAA |
| | human 18S | | CAGGGCUGACCGGGUUGGUUUU |
| | CasM.26 crRNA targeting | 414 | GCCACCCCAAAAAUGAAGGGGACUAAAA |
| | human GAPDH | | CACGCCGCUGCGGGGUGGGCCC |
| | CasM.26 crRNA targeting | 415 | GCCACCCCAAAAAUGAAGGGGACUAAAA |
| | human GAPDH | | CAAGCCUGGGUGCGGGGCUCCG |
| | CasM.26 crRNA targeting | 416 | GCCACCCCAAAAAUGAAGGGGACUAAAA |
| | human GAPDH | | CAGUGUGUGGGGAGAAGGGAUG |
| | CasM.26 crRNA targeting | 417 | GCCACCCCAAAAAUGAAGGGGACUAAAA |
| | human HRPT1 | | CACAAAGUGCUGGGAUUACAGG |
| | CasM.26 crRNA targeting | 418 | GCCACCCCAAAAAUGAAGGGGACUAAAA |
| | human HRPT1 | | CACCCAGCACUUUGGGAGGCUG |
| | CasM.26 crRNA targeting | 419 | GCCACCCCAAAAAUGAAGGGGACUAAAA |
| | human HRPT1 | | CAAAAGUGCUGGGAUUACAGGC |
| | CasM.26 crRNA targeting | 420 | GCCACCCCAAAAAUGAAGGGGACUAAAA |
| | human GUSB | | CAGCCUCCCAAAGUGCUGGGAU |
| | CasM.26 crRNA targeting | 421 | GCCACCCCAAAAAUGAAGGGGACUAAAA |
| | human GUSB | | CAAGCACUUUGGGAGGCCAAGG |
| | CasM.26 crRNA targeting | 422 | GCCACCCCAAAAAUGAAGGGGACUAAAA |
| | human RNaseP | | CACUCCCCGAAGGGCGGGGUCC |
| | CasM.26 crRNA targeting | 423 | GCCACCCCAAAAAUGAAGGGGACUAAAA |
| | human RNaseP | | CAGCCCCUCCCCGAAGGGCGGG |
| | CasM.26 crRNA targeting | 424 | GCCACCCCAAAAAUGAAGGGGACUAAAA |
| | human RNaseP | | CAGGAGGCAUCCGCCGGGCCCC |
| | CasM.26 crRNA targeting | 425 | GCCACCCCAAAAAUGAAGGGGACUAAAA |
| | SARS-CoV-2 S-gene 156/157 | | CAUCUGAACUCACUUUCCAUCC |
| | WT | | |
| | CasM.26 crRNA targeting | 426 | GCCACCCCAAAAAUGAAGGGGACUAAAA |
| | SARS-CoV-2 S-gene | | CACCACGUUCCAUCCAACUUUU |
| | del 156/157 (delta variant) | | |
| | CasM.26 crRNA targeting | 427 | GCCACCCCAAAAAUGAAGGGGACUAAAA |
| | SARS-CoV-2 S-gene 241/243 | | CAGCAAGUAAAGUUUGAAACCU |
| | WT | | |
| | CasM.26 crRNA targeting | 428 | GCCACCCCAAAAAUGAAGGGGACUAAAA |
| | SARS-CoV-2 S-gene | | CAGUUUAAAACCUAGUGAUGUU |
| | del241/243 (beta variant) | | |
| | CasM.26 crRNA targeting | 429 | GCCACCCCAAAAAUGAAGGGGACUAAAA |
| | SARS-CoV-2 S-gene 69/70 WT | | CAACAUGUAUAGCAUGGAACCA |
| | CasM.26 crRNA targeting | 430 | GCCACCCCAAAAAUGAAGGGGACUAAAA |
| | SARS-CoV-2 S-gene del69/70 | | CAGAUAUCAUGGAACCAAGUAA |
| | (alpha variant) | | |
| | CasM.26 crRNA targeting | 431 | GCCACCCCAAAAAUGAAGGGGACUAAAA |
| | SARS-CoV-2 S-gene 144/145 | | CAUAAACACCCAAAAAUGGAUC |
| | WT | | |
| | CasM.26 crRNA targeting | 432 | GCCACCCCAAAAAUGAAGGGGACUAAAA |
| | SARS-CoV-2 S-gene | | CAGUAAACACCCAAAAAUGGAU |
| | del 144/145 (alpha variant) | | |
| | CasM.26 crRNA targeting | 433 | GCCACCCCAAAAAUGAAGGGGACUAAAA |
| | SARS-CoV-2 S-gene V1176 | | CAACUGAAGCAUUAAUGCCAGA |
| | WT | | |
| | CasM.26 crRNA targeting | 434 | GCCACCCCAAAAAUGAAGGGGACUAAAA |
| | SARS-CoV-2 S-gene V1176F | | CAAAGUAAGCAUUAAUGCCAGA |
| | (gamma variant) | | |
| | CasM.26 crRNA targeting | 435 | GCCACCCCAAAAAUGAAGGGGACUAAAA |
| | SARS-CoV-2 S-gene D614 WT | | CACAUCCUGAUAAAGAACAGCA |
| | CasM.26 crRNA targeting | 436 | GCCACCCCAAAAAUGAAGGGGACUAAAA |
| | SARS-CoV-2 S-gene D614G | | CACACCUUGAUAAAGAACAGCA |
| | (beta variant) | | |
| | CasM.26 crRNA targeting | 437 | GCCACCCCAAAAAUGAAGGGGACUAAAA |
| | SARS-CoV-2 S-gene T19 WT | | CAUUGUAAGAUUAACACACUGA |
| | CasM.26 crRNA targeting | 438 | GCCACCCCAAAAAUGAAGGGGACUAAAA |
| | SARS-CoV-2 S-gene T19R | | CAUUCUGAGAUUAACACACUGA |
| | (delta variant) | | |
| | CasM.26 crRNA targeting | 439 | GCCACCCCAAAAAUGAAGGGGACUAAAA |
| | SARS-CoV-2 S-gene D80 WT | | CAUAUCAAACCUCUUAGUACCA |
| | CasM.26 crRNA targeting | 440 | GCCACCCCAAAAAUGAAGGGGACUAAAA |
| | SARS-CoV-2 S-gene D80A | | CAUAGCGAACCUCUUAGUACCA |
| | (beta variant) | | |
| | CasM.26 crRNA targeting | 441 | GCCACCCCAAAAAUGAAGGGGACUAAAA |
| | SARS-CoV-2 S-gene D215 WT | | CAGAUCACGCACUAAAUUAAUA |
| | CasM.26 crRNA targeting | 442 | GCCACCCCAAAAAUGAAGGGGACUAAAA |
| | SARS-CoV-2 S-gene D215G | | CAGACCGCGCACUAAAUUAAUA |
| | (beta variant) | | |
| | CasM.26 crRNA targeting | 443 | GCCACCCCAAAAAUGAAGGGGACUAAAA |
| | SARS-CoV-2 S-gene E484 WT | | CAUUCAACACCAUUACAAGGUG |
| | CasM.26 crRNA targeting | 444 | GCCACCCCAAAAAUGAAGGGGACUAAAA |
| | SARS-CoV-2 S-gene E484K | | CAUUUAGCACCAUUACAAGGUG |
| | (gamma variant) | | |
| | CasM.26 crRNA targeting | 445 | GCCACCCCAAAAAUGAAGGGGACUAAAA |
| | SARS-CoV-2 S-gene P681 WT | | CAGAGGAGAAUUAGUCUGAGUC |
| | CasM.26 crRNA targeting | 446 | GCCACCCCAAAAAUGAAGGGGACUAAAA |
| | SARS-CoV-2 S-gene P681H | | CAGAUGGGAAUUAGUCUGAGUC |
| | (alpha variant) | | |
| | CasM.26 crRNA targeting | 447 | GCCACCCCAAAAAUGAAGGGGACUAAAA |
| | SARS-CoV-2 S-gene P681 WT | | CAGAGGGGAAUUAGUCUGAGUC |
| | CasM.26 crRNA targeting | 448 | GCCACCCCAAAAAUGAAGGGGACUAAAA |
| | SARS-CoV-2 S-gene P681R | | CAGACGUGAAUUAGUCUGAGUC |
| | (delta variant) | | |
| | CasM.26 crRNA targeting | 449 | GCCACCCCAAAAAUGAAGGGGACUAAAA |
| | SARS-CoV-2 S-gene A701 WT | | CACUGCACCAAGUGACAUAGUG |
| | CasM.26 crRNA targeting | 450 | GCCACCCCAAAAAUGAAGGGGACUAAAA |
| | SARS-CoV-2 S-gene A701V | | CACUACGCCAAGUGACAUAGUG |
| | (beta variant) | | |
| | CasM.26 crRNA targeting | 451 | GCCACCCCAAAAAUGAAGGGGACUAAAA |
| | SARS-CoV-2 S-gene T716 WT | | CAUUGUGGGUAUGGCAAUAGAG |
| | CasM.26 crRNA targeting | 452 | GCCACCCCAAAAAUGAAGGGGACUAAAA |
| | SARS-CoV-2 S-gene T716I | | CAUUAUUGGUAUGGCAAUAGAG |
| | (alpha variant) | | |
| | CasM.26 crRNA targeting | 453 | GCCACCCCAAAAAUGAAGGGGACUAAAA |
| | SARS-CoV-2 S-gene S982 WT | | CAUGAAAGGAUAUCAUUUAAAA |
| | CasM.26 crRNA targeting | 454 | GCCACCCCAAAAAUGAAGGGGACUAAAA |
| | SARS-CoV-2 S-gene S982A | | CAUGCACGGAUAUCAUUUAAAA |
| | (alpha variant) | | |
| | CasM.26 crRNA targeting | 455 | GCCACCCCAAAAAUGAAGGGGACUAAAA |
| | SARS-CoV-2 S-gene D1118 | | CAGUCUGUAGUAAUGAUUUGUG |
| | WT | | |
| | CasM.26 crRNA targeting | 456 | GCCACCCCAAAAAUGAAGGGGACUAAAA |
| | SARS-CoV-2 S-gene D1118H | | CAGUGUAUAGUAAUGAUUUGUG |
| | (alpha variant) | | |
| | CasM.26 crRNA targeting | 457 | GCCACCCCAAAAAUGAAGGGGACUAAAA |
| | SARS-CoV-2 S-gene A570 WT | | CACAGCAAUGUCUCUGCCAAAU |
| | CasM.26 crRNA targeting | 458 | GCCACCCCAAAAAUGAAGGGGACUAAAA |
| | SARS-CoV-2 S-gene A570D | | CACAUCGAUGUCUCUGCCAAAU |
| | (alpha variant) | | |
| | CasM.26 crRNA targeting | 459 | GCCACCCCAAAAAUGAAGGGGACUAAAA |
| | PSMB2 | | CACGGAGAGGAUUAUGGAAUGC |
| | CasM.26 crRNA targeting | 460 | GCCACCCCAAAAAUGAAGGGGACUAAAA |
| | PSMB2 | | CAAGUUAGGUUCGGAGAGGAUU |
| | CasM.26 crRNA targeting | 461 | GCCACCCCAAAAAUGAAGGGGACUAAAA |
| | PSMB2 | | CAAAGUGGGGAGUUAGGUUCGG |
| | CasM.26 crRNA targeting | 462 | GCCACCCCAAAAAUGAAGGGGACUAAAA |
| | RPL32 | | CACGGCCAUGAUGCCGAGAAGG |
| | CasM.26 crRNA targeting | 463 | GCCACCCCAAAAAUGAAGGGGACUAAAA |
| | RPL32 | | CAGGGCGGCCAUGAUGCCGAGA |
| | CasM.26 crRNA targeting | 464 | GCCACCCCAAAAAUGAAGGGGACUAAAA |
| | RPL32 | | CACAUGAUGCCGAGAAGGAGAU |
| | CasM.26 crRNA targeting | 465 | GCCACCCCAAAAAUGAAGGGGACUAAAA |
| | VPS29 | | CAUUCUCAUCGAAGUCUCCUCU |
| | CasM.26 crRNA targeting | 466 | GCCACCCCAAAAAUGAAGGGGACUAAAA |
| | VPS29 | | CAUCUCAUCGAAGUCUCCUCUC |
| | CasM.26 crRNA targeting | 467 | GCCACCCCAAAAAUGAAGGGGACUAAAA |
| | VPS29 | | CAGAUUCUCAUCGAAGUCUCCU |
| | CasM.26 crRNA targeting VCP | 468 | GCCACCCCAAAAAUGAAGGGGACUAAAA |
| | | | CAGCGACCAAAUCGCCGUAGAG |
| | CasM.26 crRNA targeting VCP | 469 | GCCACCCCAAAAAUGAAGGGGACUAAAA |
| | | | CAAAGCGACCAAAUCGCCGUAG |
| | CasM.26 crRNA targeting VCP | 470 | GCCACCCCAAAAAUGAAGGGGACUAAAA |
| | | | CAUGUCAAAGCGACCAAAUCGC |
| | CasM.26 crRNA targeting | 471 | GCCACCCCAAAAAUGAAGGGGACUAAAA |
| | SNRPD3 | | CAUUCCUCUUGCGGCCACUUGG |
| | CasM.26 crRNA targeting | 472 | GCCACCCCAAAAAUGAAGGGGACUAAAA |
| | SNRPD3 | | CAGCGGCCACUUGGGCCUUGAG |
| | CasM.26 crRNA targeting | 473 | GCCACCCCAAAAAUGAAGGGGACUAAAA |
| | SNRPD3 | | CACGGCCACUUGGGCCUUGAGA |
| | CasM.26 crRNA targeting GPI | 474 | GCCACCCCAAAAAUGAAGGGGACUAAAA |
| | | | CAACGGCUCGACCCUCGGUGUA |
| | CasM.26 crRNA targeting GPI | 475 | GCCACCCCAAAAAUGAAGGGGACUAAAA |
| | | | CACGGCUCGACCCUCGGUGUAG |
| | CasM.26 crRNA targeting GPI | 476 | GCCACCCCAAAAAUGAAGGGGACUAAAA |
| | | | CAUCGACCCUCGGUGUAGUUGA |
| | CasM.26 crRNA targeting | 477 | GCCACCCCAAAAAUGAAGGGGACUAAAA |
| | RPL13 | | CAUCUUAUAGACGUUCCGGACG |
| | CasM.26 crRNA targeting | 478 | GCCACCCCAAAAAUGAAGGGGACUAAAA |
| | RPL13 | | CAUAGACGUUCCGGACGGGCAU |
| | CasM.26 crRNA targeting | 479 | GCCACCCCAAAAAUGAAGGGGACUAAAA |
| | RPL13 | | CAUUAUAGACGUUCCGGACGGG |
| | CasM.26 crRNA targeting | 480 | GCCACCCCAAAAAUGAAGGGGACUAAAA |
| | RPL19 | | CAGAUACAUGUGGCGAUCGAUC |
| | CasM.26 crRNA targeting | 481 | GCCACCCCAAAAAUGAAGGGGACUAAAA |
| | RPL19 | | CAGUGAUACAUGUGGCGAUCGA |
| | CasM.26 crRNA targeting | 482 | GCCACCCCAAAAAUGAAGGGGACUAAAA |
| | RPL19 | | CACCGCUUACCUAUGCCCAUGU |
| | CasM.26 crRNA targeting | 483 | GCCACCCCAAAAAUGAAGGGGACUAAAA |
| | REEP5 | | CACCGAUGACACCAAGAGCGAU |
| | CasM.26 crRNA targeting | 484 | GCCACCCCAAAAAUGAAGGGGACUAAAA |
| | REEP5 | | CAGUCCGAUGACACCAAGAGCG |
| | CasM.26 crRNA targeting | 485 | GCCACCCCAAAAAUGAAGGGGACUAAAA |
| | REEP5 | | CAACGGUAGCUUUCUUCGCUUC |
| | CasM.26 crRNA targeting | 486 | GCCACCCCAAAAAUGAAGGGGACUAAAA |
| | RAB7A | | CAUCAAACGCGGCCGCUGCGCU |
| | CasM.26 crRNA targeting | 487 | GCCACCCCAAAAAUGAAGGGGACUAAAA |
| | RAB7A | | CAGGUCAUCCUUCAAACGCGGC |
| | CasM.26 crRNA targeting | 488 | GCCACCCCAAAAAUGAAGGGGACUAAAA |
| | RAB7A | | CACUUCAAACGCGGCCGCUGCG |
| | CasM.26 crRNA targeting | 489 | GCCACCCCAAAAAUGAAGGGGACUAAAA |
| | RPL31 | | CAGCGGGAGCCAUUCUGCAGCG |
| | CasM.26 crRNA targeting | 490 | GCCACCCCAAAAAUGAAGGGGACUAAAA |
| | RPL31 | | CAGGAGCCAUUCUGCAGCGUCC |
| | CasM.26 crRNA targeting | 491 | GCCACCCCAAAAAUGAAGGGGACUAAAA |
| | RPL31 | | CAGCCAUUCUGCAGCGUCCAAG |
| | CasM.26 crRNA targeting | 492 | GCCACCCCAAAAAUGAAGGGGACUAAAA |
| | RPL37A | | CAACGUUUGGCCAUGUCGCCGC |
| | CasM.26 crRNA targeting | 493 | GCCACCCCAAAAAUGAAGGGGACUAAAA |
| | RPL37A | | CAUACGUUUGGCCAUGUCGCCG |
| | CasM.26 crRNA targeting | 494 | GCCACCCCAAAAAUGAAGGGGACUAAAA |
| | RPL37A | | CAUUGGCCAUGUCGCCGCGACC |
| | CasM.26 crRNA targeting | 495 | GCCACCCCAAAAAUGAAGGGGACUAAAA |
| | RPL30 | | CAGGGACGGCCCCCAACCAACA |
| | CasM.26 crRNA targeting | 496 | GCCACCCCAAAAAUGAAGGGGACUAAAA |
| | RPL30 | | CAGACUCCAGCGACUUUUUCGU |
| | CasM.26 crRNA targeting | 497 | GCCACCCCAAAAAUGAAGGGGACUAAAA |
| | RPL30 | | CACGGCCCCCAACCAACAGCAG |
| | CasM.26 crRNA targeting | 498 | GCCACCCCAAAAAUGAAGGGGACUAAAA |
| | RPS6 | | CACGCUUCUCCUUAGCCUCCUU |
| | CasM.26 crRNA targeting | 499 | GCCACCCCAAAAAUGAAGGGGACUAAAA |
| | RPS6 | | CAUUCUCCUUAGCCUCCUUCAU |
| | CasM.26 crRNA targeting | 500 | GCCACCCCAAAAAUGAAGGGGACUAAAA |
| | RPS6 | | CAUCUCCUUAGCCUCCUUCAUU |
| | CasM.26 crRNA targeting | 501 | GCCACCCCAAAAAUGAAGGGGACUAAAA |
| | RPS29 | | CAAACAGACACGACAAGAGCGA |
| | CasM.26 crRNA targeting | 502 | GCCACCCCAAAAAUGAAGGGGACUAAAA |
| | RPS29 | | CAUGAACAGACACGACAAGAGC |
| | CasM.26 crRNA targeting | 503 | GCCACCCCAAAAAUGAAGGGGACUAAAA |
| | RPS29 | | CACACGACAAGAGCGAGAACCC |
| | CasM.26 crRNA targeting | 504 | GCCACCCCAAAAAUGAAGGGGACUAAAA |
| | RPL27A | | CAACUUUGUAGUAGCCCGAUCG |
| | CasM.26 crRNA targeting | 505 | GCCACCCCAAAAAUGAAGGGGACUAAAA |
| | RPL27A | | CAGUAGUAGCCCGAUCGCACCA |
| | CasM.26 crRNA targeting | 506 | GCCACCCCAAAAAUGAAGGGGACUAAAA |
| | RPL27A | | CAGCCUAUGCGGCCGUGGCCGU |
| | CasM.26 crRNA targeting | 507 | GCCACCCCAAAAAUGAAGGGGACUAAAA |
| | RPS10 | | CAUCUGGCAAGUCUCCGCCACU |
| | CasM.26 crRNA targeting | 508 | GCCACCCCAAAAAUGAAGGGGACUAAAA |
| | RPS10 | | CAUAGGCAUCAACAUCUGGCAA |
| | CasM.26 crRNA targeting | 509 | GCCACCCCAAAAAUGAAGGGGACUAAAA |
| | RPS10 | | CAACAUCUGGCAAGUCUCCGCC |
| | CasM.26 crRNA targeting | 510 | GCCACCCCAAAAAUGAAGGGGACUAAAA |
| | RPS27 | | CAGAGAUCCUUUGCGAGCGGGG |
| | CasM.26 crRNA targeting | 511 | GCCACCCCAAAAAUGAAGGGGACUAAAA |
| | RPS27 | | CAGAUCCUUUGCGAGCGGGGAG |
| | CasM.26 crRNA targeting | 512 | GCCACCCCAAAAAUGAAGGGGACUAAAA |
| | RPS27 | | CAUUGCGAGCGGGGAGAGCUCG |
| | CasM.26 crRNA targeting | 513 | GCCACCCCAAAAAUGAAGGGGACUAAAA |
| | ACTB | | CAAUGAUCUUGAUCUUCAUUGU |
| | CasM.26 crRNA targeting | 514 | GCCACCCCAAAAAUGAAGGGGACUAAAA |
| | ACTB | | CAACGCCCUGGUGCCUGGGGCG |
| | CasM.26 crRNA targeting | 515 | GCCACCCCAAAAAUGAAGGGGACUAAAA |
| | ACTB | | CAGCUGGCGGCGGGUGUGGACG |
| | CasM.26 crRNA targeting | 516 | GCCACCCCAAAAAUGAAGGGGACUAAAA |
| | ATP5PB | | CACCGAGUACAUAGGGUCCUGU |
| | CasM.26 crRNA targeting | 517 | GCCACCCCAAAAAUGAAGGGGACUAAAA |
| | ATP5PB | | CACGAGUACAUAGGGUCCUGUU |
| | CasM.26 crRNA targeting | 518 | GCCACCCCAAAAAUGAAGGGGACUAAAA |
| | ATP5PB | | CAAGAGGGGGCCGCUGUGGCGG |
| | CasM.26 crRNA targeting | 519 | GCCACCCCAAAAAUGAAGGGGACUAAAA |
| | NDUFB4 | | CAUUUUCGAUGAGCCCUCGGCG |
| | CasM.26 crRNA targeting | 520 | GCCACCCCAAAAAUGAAGGGGACUAAAA |
| | NDUFB4 | | CAGCAAGGCAGGAUUUUCGAUG |
| | CasM.26 crRNA targeting | 521 | GCCACCCCAAAAAUGAAGGGGACUAAAA |
| | NDUFB4 | | CAUUCGAUGAGCCCUCGGCGGU |
| | CasM.26 crRNA targeting | 522 | GCCACCCCAAAAAUGAAGGGGACUAAAA |
| | ATP5MG | | CAAGCAGCGUUCACCAGCGCCG |
| | CasM.26 crRNA targeting | 523 | GCCACCCCAAAAAUGAAGGGGACUAAAA |
| | ATP5MG | | CAGCGUUCACCAGCGCCGGGGU |
| | CasM.26 crRNA targeting | 524 | GCCACCCCAAAAAUGAAGGGGACUAAAA |
| | ATP5MG | | CACGAGUAAGUCACAGCAGCGU |
| | CasM.26 crRNA targeting | 525 | GCCACCCCAAAAAUGAAGGGGACUAAAA |
| | EMC7 | | CAGUCUUAAGGAAACCGACGUG |
| | CasM.26 crRNA targeting | 526 | GCCACCCCAAAAAUGAAGGGGACUAAAA |
| | EMC7 | | CAUCAUCAUAACCAUUGGGUUC |
| | CasM.26 crRNA targeting | 527 | GCCACCCCAAAAAUGAAGGGGACUAAAA |
| | EMC7 | | CAGACUGCUCCAUUUCCCGUCU |
| | CasM.26 crRNA targeting | 528 | GCCACCCCAAAAAUGAAGGGGACUAAAA |
| | C1orf43 | | CAAGACCCCUGUCCCAUAGCGG |
| | CasM.26 crRNA targeting | 529 | GCCACCCCAAAAAUGAAGGGGACUAAAA |
| | Clorf43 | | CACAUUCUGGCCAAAGACCCCU |
| | CasM.26 crRNA targeting | 530 | GCCACCCCAAAAAUGAAGGGGACUAAAA |
| | Clorf43 | | CAUGGCCAAAGACCCCUGUCCC |
| | CasM.26 crRNA targeting | 531 | GCCACCCCAAAAAUGAAGGGGACUAAAA |
| | PRDX1 | | CAUAAAAAGGCCCCUGAACGAG |
| | CasM.26 crRNA targeting | 532 | GCCACCCCAAAAAUGAAGGGGACUAAAA |
| | PRDX1 | | CAGAUAAAAAGGCCCCUGAACG |
| | CasM.26 crRNA targeting | 533 | GCCACCCCAAAAAUGAAGGGGACUAAAA |
| | PRDX1 | | CAAAAAGGCCCCUGAACGAGAU |
| | CasM.26 crRNA targeting | 534 | GCCACCCCAAAAAUGAAGGGGACUAAAA |
| | ATP5MF | | CAUCGUGCUCUCUUUGAAACGC |
| | CasM.26 crRNA targeting | 535 | GCCACCCCAAAAAUGAAGGGGACUAAAA |
| | ATP5MF | | CACACUGGUACUGGGGCCGGAC |
| | CasM.26 crRNA targeting | 536 | GCCACCCCAAAAAUGAAGGGGACUAAAA |
| | ATP5MF | | CACGGAGCCGCUCGUGCUCUCU |
| | CasM.26 crRNA targeting | 537 | GCCACCCCAAAAAUGAAGGGGACUAAAA |
| | NDUFB3 | | CAAUUGCGGCCCCAUGGAUCCC |
| | CasM.26 crRNA targeting | 538 | GCCACCCCAAAAAUGAAGGGGACUAAAA |
| | NDUFB3 | | CAAUCUCCAAGCUUCAUUGCGG |
| | CasM.26 crRNA targeting | 539 | GCCACCCCAAAAAUGAAGGGGACUAAAA |
| | NDUFB3 | | CAAAGCUUCAUUGCGGCCCCAU |
| | CasM.26 crRNA targeting | 540 | GCCACCCCAAAAAUGAAGGGGACUAAAA |
| | NDUFB8 | | CAGUGCCAGUGCAUCGGUUCAC |
| | CasM.26 crRNA targeting | 541 | GCCACCCCAAAAAUGAAGGGGACUAAAA |
| | NDUFB8 | | CAUCGGGUAGUCGCCAUACCCC |
| | CasM.26 crRNA targeting | 542 | GCCACCCCAAAAAUGAAGGGGACUAAAA |
| | NDUFB8 | | CACGCCAUACCCCAUGCCAUCA |
| | CasM.26 crRNA targeting | 543 | GCCACCCCAAAAAUGAAGGGGACUAAAA |
| | CHMP2A | | CAAGGUUCGACAGCUCAUCUGU |
| | CasM.26 crRNA targeting | 544 | GCCACCCCAAAAAUGAAGGGGACUAAAA |
| | CHMP2A | | CAUUGAGGGGAGGUUCGACAGC |
| | CasM.26 crRNA targeting | 545 | GCCACCCCAAAAAUGAAGGGGACUAAAA |
| | CHMP2A | | CAGGGGAGGUUCGACAGCUCAU |
| | CasM.26 crRNA targeting | 546 | GCCACCCCAAAAAUGAAGGGGACUAAAA |
| | ATP5ME | | CAGCGGCCGAGCUUGAUGAGCG |
| | CasM.26 crRNA targeting | 547 | GCCACCCCAAAAAUGAAGGGGACUAAAA |
| | ATP5ME | | CAGAGUAGCGGCCGAGCUUGAU |
| | CasM.26 crRNA targeting | 548 | GCCACCCCAAAAAUGAAGGGGACUAAAA |
| | ATP5ME | | CAAAUUGUAGCGCGUGGCUCCG |
| | CasM.26 crRNA targeting | 549 | GCCACCCCAAAAAUGAAGGGGACUAAAA |
| | PSMB4 | | CACGAUUUGAAACCGGUUGUAA |
| | CasM.26 crRNA targeting | 550 | GCCACCCCAAAAAUGAAGGGGACUAAAA |
| | PSMB4 | | CAGAAACCGGUUGUAAGAACGG |
| | CasM.26 crRNA targeting | 551 | GCCACCCCAAAAAUGAAGGGGACUAAAA |
| | PSMB4 | | CAGGGUUCUGGGUCCGCGUGAU |
| | CasM.26 crRNA targeting | 552 | GCCACCCCAAAAAUGAAGGGGACUAAAA |
| | YWHAG | | CAUGUCACGUUCUUCAUGGCCG |
| | CasM.26 crRNA targeting | 553 | GCCACCCCAAAAAUGAAGGGGACUAAAA |
| | YWHAG | | CACAUUCAGCUCUGUCACGUUC |
| | CasM.26 crRNA targeting | 554 | GCCACCCCAAAAAUGAAGGGGACUAAAA |
| | YWHAG | | CAUCUGUCACGUUCUUCAUGGC |
| | CasM.26 crRNA targeting 28S | 555 | GCCACCCCAAAAAUGAAGGGGACUAAAA |
| | rRNA | | CACACUCGGGGGGGGUUUCGGU |
| | CasM.26 crRNA targeting 28S | 556 | GCCACCCCAAAAAUGAAGGGGACUAAAA |
| | rRNA | | CACUCCGACGCACACCACACGC |
| | CasM.26 crRNA targeting 28S | 557 | GCCACCCCAAAAAUGAAGGGGACUAAAA |
| | rRNA | | CAGUGCGUCGGGUCUGCGAGAG |
| | CasM.26 crRNA targeting | 558 | GCCACCCCAAAAAUGAAGGGGACUAAAA |
| | AMR gene IMP | | CAAACGAAGUAUGAACAUAAAC |
| | CasM.26 crRNA targeting | 559 | GCCACCCCAAAAAUGAAGGGGACUAAAA |
| | AMR gene IMP | | CAAAACGAAGUAUGAACAUAAA |
| | CasM.26 crRNA targeting | 560 | GCCACCCCAAAAAUGAAGGGGACUAAAA |
| | AMR gene IMP | | CAAAUUUUUAAAUCUGGCAAAG |
| | CasM.26 crRNA targeting | 561 | GCCACCCCAAAAAUGAAGGGGACUAAAA |
| | AMR gene IMP | | CAUAGAUUGAGAAUUAAGCCAC |
| | CasM.26 crRNA targeting | 562 | GCCACCCCAAAAAUGAAGGGGACUAAAA |
| | AMR gene IMP | | CAAGAUUGAGAAUUAAGCCACU |
| | CasM.26 crRNA targeting | 563 | GCCACCCCAAAAAUGAAGGGGACUAAAA |
| | AMR gene IMP | | CAAUAGAUUGAGAAUUAAGCCA |
| | CasM.26 crRNA targeting | 564 | GCCACCCCAAAAAUGAAGGGGACUAAAA |
| | AMR gene CTX-M-1 | | CAUUUAGCCGCCGACGCUAAUA |
| | CasM.26 crRNA targeting | 565 | GCCACCCCAAAAAUGAAGGGGACUAAAA |
| | AMR gene CTX-M-1 | | CAAGUGGUAUCACGCGGAUCGC |
| | CasM.26 crRNA targeting | 566 | GCCACCCCAAAAAUGAAGGGGACUAAAA |
| | AMR gene CTX-M-1 | | CAGACGAUUUUAGCCGCCGACG |
| | CasM.26 crRNA targeting | 567 | GCCACCCCAAAAAUGAAGGGGACUAAAA |
| | AMR gene CTX-M-2 | | CACGCUGCCGGUUUUAUCGCCC |
| | CasM.26 crRNA targeting | 568 | GCCACCCCAAAAAUGAAGGGGACUAAAA |
| | AMR gene CTX-M-2 | | CACUGCCGGUUUUAUCGCCCAC |
| | CasM.26 crRNA targeting | 569 | GCCACCCCAAAAAUGAAGGGGACUAAAA |
| | AMR gene CTX-M-2 | | CAUUUCAGGGUCUGCGCCAUCG |
| | CasM.26 crRNA targeting | 570 | GCCACCCCAAAAAUGAAGGGGACUAAAA |
| | AMR gene CTX-M-25 | | CACGCGAUAUCAUUCGUCGUAC |
| | CasM.26 crRNA targeting | 571 | GCCACCCCAAAAAUGAAGGGGACUAAAA |
| | AMR gene CTX-M-25 | | CAAACCGCGAUAUCAUUCGUCG |
| | CasM.26 crRNA targeting | 572 | GCCACCCCAAAAAUGAAGGGGACUAAAA |
| | AMR gene CTX-M-25 | | CAAACGAGCGGCGCGCGACCUU |
| | CasM.26 crRNA targeting | 573 | GCCACCCCAAAAAUGAAGGGGACUAAAA |
| | AMR gene CTX-M-8 | | CAGCGAUGUCAUUCGUCGUACC |
| | CasM.26 crRNA targeting | 574 | GCCACCCCAAAAAUGAAGGGGACUAAAA |
| | AMR gene CTX-M-8 | | CAACGGCGAUGUCAUUCGUCGU |
| | CasM.26 crRNA targeting | 575 | GCCACCCCAAAAAUGAAGGGGACUAAAA |
| | AMR gene CTX-M-8 | | CAGACGAUUUUCGCGGCAGCCG |
| | CasM.26 crRNA targeting | 576 | GCCACCCCAAAAAUGAAGGGGACUAAAA |
| | AMR gene CTX-M-9 | | CACAGUCCACGACGUCGGUAAG |
| | CasM.26 crRNA targeting | 577 | GCCACCCCAAAAAUGAAGGGGACUAAAA |
| | AMR gene CTX-M-9 | | CACCACGACGUCGGUAAGCCGG |
| | CasM.26 crRNA targeting | 578 | GCCACCCCAAAAAUGAAGGGGACUAAAA |
| | AMR gene CTX-M-9 | | CAGACGUCGGUAAGCCGGCCCG |
| | CasM.26 crRNA targeting Lujo | 579 | GCCACCCCAAAAAUGAAGGGGACUAAAA |
| | mammarenavirus L segment | | CAUGAUGAGAGACUAGCUCCCC |
| | CasM.26 crRNA targeting Lujo | 580 | GCCACCCCAAAAAUGAAGGGGACUAAAA |
| | mammarenavirus L segment | | CAACUCCUGCAGCUUAUCUCAA |
| | CasM.26 crRNA targeting Lujo | 581 | GCCACCCCAAAAAUGAAGGGGACUAAAA |
| | mammarenavirus L segment | | CAGAAUGAACUCCUGCAGCUUA |
| | CasM.26 crRNA targeting Lujo | 582 | GCCACCCCAAAAAUGAAGGGGACUAAAA |
| | mammarenavirus S segment | | CAGCUUGUCGACAUCUUGUCUC |
| | CasM.26 crRNA targeting Lujo | 583 | GCCACCCCAAAAAUGAAGGGGACUAAAA |
| | mammarenavirus S segment | | CACUUCUGCGCUUGUCGACAUC |
| | CasM.26 crRNA targeting Lujo | 584 | GCCACCCCAAAAAUGAAGGGGACUAAAA |
| | mammarenavirus S segment | | CAGUCGACAUCUUGUCUCACAA |
| | CasM.26 crRNA targeting | 585 | GCCACCCCAAAAAUGAAGGGGACUAAAA |
| | Lassa mammarenavirus | | CACGCCCCCGUGACCCACCGCC |
| | CasM.26 crRNA targeting | 586 | GCCACCCCAAAAAUGAAGGGGACUAAAA |
| | Lassa mammarenavirus | | CAACGCCCCCGUGACCCACCGC |
| | CasM.26 crRNA targeting | 587 | GCCACCCCAAAAAUGAAGGGGACUAAAA |
| | Lassa mammarenavirus | | CAGGACGCCCCCGUGACCCACC |
| | CasM.26 crRNA targeting | 588 | GCCACCCCAAAAAUGAAGGGGACUAAAA |
| | Argentinian mammarenavirus | | CACGAUGGUUGGCCUUAUAUUG |
| | (Junin arenavirus) | | |
| | CasM.26 crRNA targeting | 589 | GCCACCCCAAAAAUGAAGGGGACUAAAA |
| | Argentinian mammarenavirus | | CAGUCUUUCUGGCGAUGGUUGG |
| | (Junin arenavirus) | | |
| | CasM.26 crRNA targeting | 590 | GCCACCCCAAAAAUGAAGGGGACUAAAA |
| | Argentinian mammarenavirus | | CAUGGCGAUGGUUGGCCUUAUA |
| | (Junin arenavirus) | | |
| | CasM.26 crRNA targeting | 591 | GCCACCCCAAAAAUGAAGGGGACUAAAA |
| | Machupo mammarenavirus | | CAAUCAAGAAUCGCCUAGGAUC |
| | CasM.26 crRNA targeting | 592 | GCCACCCCAAAAAUGAAGGGGACUAAAA |
| | Machupo mammarenavirus | | CAAAUUGAUGGAGCUUGCAUCA |
| | CasM.26 crRNA targeting | 593 | GCCACCCCAAAAAUGAAGGGGACUAAAA |
| | Machupo mammarenavirus | | CAGACAAUAACCUCCUGGCAUG |
| | CasM.26 crRNA targeting | 594 | GCCACCCCAAAAAUGAAGGGGACUAAAA |
| | Pseudorabies Virus | | CAAGGUCGCCGUUGAGGUCAUC |
| | CasM.26 crRNA targeting | 595 | GCCACCCCAAAAAUGAAGGGGACUAAAA |
| | Pseudorabies Virus | | CAGUCGCCGUUGAGGUCAUCGU |
| | CasM.26 crRNA targeting | 596 | GCCACCCCAAAAAUGAAGGGGACUAAAA |
| | Pseudorabies Virus | | CAUCGCCGUCGAGGUCGCCGUU |
| | CasM.26 crRNA targeting | 597 | GCCACCCCAAAAAUGAAGGGGACUAAAA |
| | Orthopox virus | | CAGUCUAUAGUCGAUUCACUAU |
| | CasM.26 crRNA targeting | 598 | GCCACCCCAAAAAUGAAGGGGACUAAAA |
| | Orthopox virus | | CAUAUAGUCGAUUCACUAUCUG |
| | CasM.26 crRNA targeting | 599 | GCCACCCCAAAAAUGAAGGGGACUAAAA |
| | Orthopox virus | | CAUAUGUCUAUAGUCGAUUCAC |
| | Cas12 Variant crRNA targeting | 600 | UAAUUUCUACUAAGUGUAGAUCGCGCGG |
| | Pseudorabies Virus | | AAUCGCAUCUACC |
| | Cas12 Variant crRNA targeting | 601 | UAAUUUCUACUAAGUGUAGAUUCUCCGU |
| | Pseudorabies Virus | | CCGCGCCGUUUUA |
| | Cas12 Variant crRNA targeting | 602 | UAAUUUCUACUAAGUGUAGAUGGGCCCA |
| | Pseudorabies Virus | | CGAUCGUCGUCCU |
| | Cas12 Variant crRNA targeting | 603 | UAAUUUCUACUAAGUGUAGAUGAGACG |
| | Orthopox virus | | UAACAAUGCACUAC |
| | Cas12 Variant crRNA targeting | 604 | UAAUUUCUACUAAGUGUAGAUGGACUA |
| | Orthopox virus | | GGGGAUGUUGAUAA |
| | Cas12 Variant crRNA targeting | 605 | UAAUUUCUACUAAGUGUAGAUUGUGAG |
| | Orthopox virus | | ACCAUCGAAGAGAG |
| | Cas12 Variant crRNA targeting | 606 | UAAUUUCUACUAAGUGUAGAUUAUGCGC |
| | AMR gene CTX-M-8 | | AGGCGAACGACGU |
| | Cas12 Variant crRNA targeting | 607 | UAAUUUCUACUAAGUGUAGAUUGGUAC |
| | AMR gene CTX-M-8 | | GACGAAUGACAUCG |
| | Cas12 Variant crRNA targeting | 608 | UAAUUUCUACUAAGUGUAGAUAUUGAC |
| | AMR gene CTX-M-8 | | ACCGCCGAUAACGC |
| | Cas12 Variant crRNA targeting | 609 | UAAUUUCUACUAAGUGUAGAUGCGCGGC |
| | AMR gene CTX-M-1 | | CGCGCUACAGUAC |
| | Cas12 Variant crRNA targeting | 610 | UAAUUUCUACUAAGUGUAGAUGCCAAAA |
| | AMR gene CTX-M-1 | | GAUCGUGCGCCGC |
| | Cas12 Variant crRNA targeting | 611 | UAAUUUCUACUAAGUGUAGAUCGUUGGC |
| | AMR gene CTX-M-1 | | GGCCCGGCUAGCG |
| | Cas12 Variant crRNA targeting | 612 | UAAUUUCUACUAAGUGUAGAUgaacgagcgg |
| | AMR gene CTX-M-25 | | cgcgcgacct |
| | Cas12 Variant crRNA targeting | 613 | UAAUUUCUACUAAGUGUAGAUUGGUAC |
| | AMR gene CTX-M-25 | | GACGAAUGAUAUCG |
| | Cas12 Variant crRNA targeting | 614 | UAAUUUCUACUAAGUGUAGAUaataaccgcga |
| | AMR gene CTX-M-25 | | tatcattcg |
| | Cas12 Variant crRNA targeting | 615 | UAAUUUCUACUAAGUGUAGAUGGGCCGG |
| | AMR gene CTX-M-9 | | CUUACCGACGUCG |
| | Cas12 Variant crRNA targeting | 616 | UAAUUUCUACUAAGUGUAGAUGGCGAU |
| | AMR gene CTX-M-9 | | GAUUCUCGCCGCUG |
| | Cas12 Variant crRNA targeting | 617 | UAAUUUCUACUAAGUGUAGAUACGACGU |
| | AMR gene CTX-M-9 | | CGGUAAGCCGGCC |
| | Cas12 Variant crRNA targeting | 618 | UAAUUUCUACUAAGUGUAGAUCCGCUGC |
| | AMR gene CTX-M-2 | | CGGUUUUAUCGCC |
| | Cas12 Variant crRNA targeting | 619 | UAAUUUCUACUAAGUGUAGAUGCGAUG |
| | AMR gene CTX-M-2 | | GCGCAGACCCUGAA |
| | Cas12 Variant crRNA targeting | 620 | UAAUUUCUACUAAGUGUAGAUGCGCUAC |
| | AMR gene CTX-M-2 | | CGGUAGUAUUGCC |
| | Cas12 Variant crRNA targeting | 621 | UAAUUUCUACUAAGUGUAGAUAAACGA |
| | AMR gene IMP | | AGUAUGAACAUAAA |
| | Cas12 Variant crRNA targeting | 622 | UAAUUUCUACUAAGUGUAGAUCAAACGA |
| | AMR gene IMP | | AGUAUGAACAUAA |
| | Cas12 Variant crRNA targeting | 623 | UAAUUUCUACUAAGUGUAGAUCAAUUU |
| | AMR gene IMP | | UUAAAUCUGGCAAA |
| | Cas12 Variant crRNA targeting | 624 | UAAUUUCUACUAAGUGUAGAUAGAAUU |
| | AMR gene IMP | | AAGCCACUCUAUUC |
| | Cas12 Variant crRNA targeting | 625 | UAAUUUCUACUAAGUGUAGAUAUAGCCG |
| | AMR gene IMP | | CGCUCCACAAACC |
| | Cas12 Variant crRNA targeting | 626 | UAAUUUCUACUAAGUGUAGAUUAUAGCC |
| | AMR gene IMP | | GCGCUCCACAAAC |
| | CasM.26 crRNA targeting | 627 | GCCACCCCAAAAAUGAAGGGGACUAAAA |
| | Stenotrophomonas maltophilia | | CACGUAUUGGCGAUGUAUCCGG |
| | alpha | | |
| | CasM.26 crRNA targeting | 628 | GCCACCCCAAAAAUGAAGGGGACUAAAA |
| | Stenotrophomonas maltophilia | | CAGCACUUCGAUUCGGGCGUCU |
| | alpha | | |
| | CasM.26 crRNA targeting | 629 | GCCACCCCAAAAAUGAAGGGGACUAAAA |
| | Stenotrophomonas maltophilia | | CACGUCUGCGAGUUGCCGUCAC |
| | alpha | | |
| | CasM.26 crRNA targeting | 630 | GCCACCCCAAAAAUGAAGGGGACUAAAA |
| | Stenotrophomonas maltophilia | | CACGUCAUUGACCGUGUUGCGG |
| | beta | | |
| | CasM.26 crRNA targeting | 631 | GCCACCCCAAAAAUGAAGGGGACUAAAA |
| | Stenotrophomonas maltophilia | | CAUUCGCCUCGUGUUCGGCUUC |
| | beta | | |
| | CasM.26 crRNA targeting | 632 | GCCACCCCAAAAAUGAAGGGGACUAAAA |
| | Stenotrophomonas maltophilia | | CAAUUGACCGUGUUGCGGCCGA |
| | beta | | |
| | CasM.26 crRNA targeting | 633 | GCCACCCCAAAAAUGAAGGGGACUAAAA |
| | Stenotrophomonas maltophilia | | CAGCUUGUACGCCCACCCAUAG |
| | gamma | | |
| | CasM.26 crRNA targeting | 634 | GCCACCCCAAAAAUGAAGGGGACUAAAA |
| | Stenotrophomonas maltophilia | | CACGGUGAUGCUUGUACGCCCA |
| | gamma | | |
| | CasM.26 crRNA targeting | 635 | GCCACCCCAAAAAUGAAGGGGACUAAAA |
| | Stenotrophomonas maltophilia | | CACUCUUCCCGACGUCCGCGCA |
| | gamma | | |
| | CasM.26 crRNA targeting | 636 | GCCACCCCAAAAAUGAAGGGGACUAAAA |
| | Staphylococcus aureus gyrA | | CACAACACGAUCCAUGAAAUCU |
| | CasM.26 crRNA targeting | 637 | GCCACCCCAAAAAUGAAGGGGACUAAAA |
| | Staphylococcus aureus gyrA | | CACUUACUUCAAUACGUCCAUC |
| | CasM.26 crRNA targeting | 638 | GCCACCCCAAAAAUGAAGGGGACUAAAA |
| | Staphylococcus aureus gyrA | | CAUCUUCAACACGAUCCAUGAA |
| | CasM.26 crRNA targeting | 639 | GCCACCCCAAAAAUGAAGGGGACUAAAA |
| | Staphylococcus aureus gyrB | | CAACGGUUUUCUACAACGUCAC |
| | CasM.26 crRNA targeting | 640 | GCCACCCCAAAAAUGAAGGGGACUAAAA |
| | Staphylococcus aureus gyrB | | CAAUCGCAUCUUCAAGUUUUAC |
| | CasM.26 crRNA targeting | 641 | GCCACCCCAAAAAUGAAGGGGACUAAAA |
| | Staphylococcus aureus gyrB | | CAGCUUCAAUCGCAUCUUCAAG |
| | CasM.26 crRNA targeting | 642 | GCCACCCCAAAAAUGAAGGGGACUAAAA |
| | Staphylococcus aureus | | CAAAUCAGCGUUGUCUUCGCUC |
| | thermonuclease | | |
| | CasM.26 crRNA targeting | 643 | GCCACCCCAAAAAUGAAGGGGACUAAAA |
| | Staphylococcus aureus | | CAGCGUUGUCUUCGCUCCAAAU |
| | thermonuclease | | |
| | CasM.26 crRNA targeting | 644 | GCCACCCCAAAAAUGAAGGGGACUAAAA |
| | Staphylococcus aureus | | CAAUUGACCUGAAUCAGCGUUG |
| | thermonuclease | | |
| | CasM.26 crRNA targeting | 645 | GCCACCCCAAAAAUGAAGGGGACUAAAA |
| | Mycobacterium tuberculosis | | CACGAGUGAAGGUUCCUCGCGU |
| | IS1081 | | |
| | CasM.26 crRNA targeting | 646 | GCCACCCCAAAAAUGAAGGGGACUAAAA |
| | Mycobacterium tuberculosis | | CAUCGCGUGAUCCUUCGGGUGG |
| | IS1081 | | |
| | CasM.26 crRNA targeting | 647 | GCCACCCCAAAAAUGAAGGGGACUAAAA |
| | Mycobacterium tuberculosis | | CAUGAAGGUUCCUCGCGUGAUC |
| | IS1081 | | |
| | CasM.26 crRNA targeting | 648 | GCCACCCCAAAAAUGAAGGGGACUAAAA |
| | Mycobacterium tuberculosis | | CAAGUUCGACCGGCGGGACGUC |
| | IS6110 | | |
| | CasM.26 crRNA targeting | 649 | GCCACCCCAAAAAUGAAGGGGACUAAAA |
| | Mycobacterium tuberculosis | | CAGGCGGGACGUCGCCGCAGUA |
| | IS6110 | | |
| | CasM.26 crRNA targeting | 650 | GCCACCCCAAAAAUGAAGGGGACUAAAA |
| | Mycobacterium tuberculosis | | CAAGCCUCGAGUUCGACCGGCG |
| | IS6110 | | |
| | Cas12 Variant crRNA targeting | 651 | UAAUUUCUACUAAGUGUAGAUGGACCAU |
| | Stenotrophomonas maltophilia | | CCGGUGAACCGCG |
| | alpha | | |
| | Cas12 Variant crRNA targeting | 652 | UAAUUUCUACUAAGUGUAGAUCUCUACC |
| | Stenotrophomonas maltophilia | | CCGAGUACCGCGC |
| | alpha | | |
| | Cas12 Variant crRNA targeting | 653 | UAAUUUCUACUAAGUGUAGAUUCCAUAU |
| | Stenotrophomonas maltophilia | | CGAAGGCGACCCG |
| | alpha | | |
| | Cas12 Variant crRNA targeting | 654 | UAAUUUCUACUAAGUGUAGAUGAGCGAC |
| | Stenotrophomonas maltophilia | | CAGUCGUGGACGG |
| | beta | | |
| | Cas12 Variant crRNA targeting | 655 | UAAUUUCUACUAAGUGUAGAUGUCCGUC |
| | Stenotrophomonas maltophilia | | GCCCGGUGCGAUC |
| | beta | | |
| | Cas12 Variant crRNA targeting | 656 | UAAUUUCUACUAAGUGUAGAUGGACAUC |
| | Stenotrophomonas maltophilia | | AUCCGCCGCUCGG |
| | beta | | |
| | Cas12 Variant crRNA targeting | 657 | UAAUUUCUACUAAGUGUAGAUCCGCCGG |
| | Stenotrophomonas maltophilia | | UGGGCCGUUUCAA |
| | gamma | | |
| | Cas12 Variant crRNA targeting | 658 | UAAUUUCUACUAAGUGUAGAUCCGACCG |
| | Stenotrophomonas maltophilia | | CCGACGAUCGCGC |
| | gamma | | |
| | Cas12 Variant crRNA targeting | 659 | UAAUUUCUACUAAGUGUAGAUCCGCGCA |
| | Stenotrophomonas maltophilia | | CCGACCCGCAUCA |
| | gamma | | |
| | Cas12 Variant crRNA targeting | 660 | UAAUUUCUACUAAGUGUAGAUGCACGUA |
| | Staphylococcus aureus gyrA | | UCGUUGGUGACGU |
| | Cas12 Variant crRNA targeting | 661 | UAAUUUCUACUAAGUGUAGAUUGCGAU |
| | Staphylococcus aureus gyrA | | GAGUGUUAUCGUUG |
| | Cas12 Variant crRNA targeting | 662 | UAAUUUCUACUAAGUGUAGAUAUGGUG |
| | Staphylococcus aureus gyrA | | CAUCAGGUAUCGCG |
| | Cas12 Variant crRNA targeting | 663 | UAAUUUCUACUAAGUGUAGAUAAGUAA |
| | Staphylococcus aureus gyrB | | CGGAUAACGGACGU |
| | Cas12 Variant crRNA targeting | 664 | UAAUUUCUACUAAGUGUAGAUGCGGUG |
| | Staphylococcus aureus gyrB | | GCGGAUACAAAGUA |
| | Cas12 Variant crRNA targeting | 665 | UAAUUUCUACUAAGUGUAGAUUAUCACU |
| | Staphylococcus aureus gyrB | | AUGAGGGCGGUAU |
| | Cas12 Variant crRNA targeting | 666 | UAAUUUCUACUAAGUGUAGAUAAGCGA |
| | Staphylococcus aureus | | UUGAUGGUGAUACG |
| | thermonuclease | | |
| | Cas12 Variant crRNA targeting | 667 | UAAUUUCUACUAAGUGUAGAUUAGUUC |
| | Staphylococcus aureus | | GUCAAGGCUUGGCU |
| | thermonuclease | | |
| | Cas12 Variant crRNA targeting | 668 | UAAUUUCUACUAAGUGUAGAUAAAGGG |
| | Staphylococcus aureus | | CAAUACGCAAAGAG |
| | thermonuclease | | |
| | Cas12 Variant crRNA targeting | 669 | UAAUUUCUACUAAGUGUAGAUAAUUGC |
| | Mycobacterium tuberculosis | | GAAGGGCGAACGCG |
| | IS6110 | | |
| | Cas12 Variant crRNA targeting | 670 | UAAUUUCUACUAAGUGUAGAUCCGCGAG |
| | Mycobacterium tuberculosis | | GGCCCCGAUGGUU |
| | IS6110 | | |
| | Cas12 Variant crRNA targeting | 671 | UAAUUUCUACUAAGUGUAGAUCAUCGAC |
| | Mycobacterium tuberculosis | | CUACUACGACCAC |
| | IS6110 | | |
| | Cas12 Variant crRNA targeting | 672 | UAAUUUCUACUAAGUGUAGAUUCAUCUU |
| | Mycobacterium tuberculosis | | AUCGACGCCGAGC |
| | IS1081 | | |
| | Cas12 Variant crRNA targeting | 673 | UAAUUUCUACUAAGUGUAGAUUCGCGUG |
| | Mycobacterium tuberculosis | | AUCCUUCGAAACG |
| | IS1081 | | |
| | Cas12 Variant crRNA targeting | 674 | UAAUUUCUACUAAGUGUAGAUGUGCCGC |
| | Mycobacterium tuberculosis | | AACCAUCGACGUC |
| | IS1081 | | |
| | CasM.26 crRNA targeting | 675 | GCCACCCCAAAAAUGAAGGGGACUAAAA |
| | Acinetobacter spp. 16S-23S | | CACCCAAACAGUCGAUGUUACC |
| | ribosomal RNA intergenic | | |
| | spacer | | |
| | CasM.26 crRNA targeting | 676 | GCCACCCCAAAAAUGAAGGGGACUAAAA |
| | Acinetobacter spp. 16S-23S | | CACGAUGUUACCUACAAGUUAG |
| | ribosomal RNA intergenic | | |
| | spacer | | |
| | CasM.26 crRNA targeting | 677 | GCCACCCCAAAAAUGAAGGGGACUAAAA |
| | Acinetobacter spp. 16S-23S | | CAUCACAUACCAAAACGCUUGA |
| | ribosomal RNA intergenic | | |
| | spacer | | |
| | CasM.26 crRNA targeting | 678 | GCCACCCCAAAAAUGAAGGGGACUAAAA |
| | Acinetobacter spp. 16S-23S | | CAGUGCACUUAAGCACCGUACA |
| | ribosomal RNA intergenic | | |
| | spacer | | |
| | CasM.26 crRNA targeting | 679 | GCCACCCCAAAAAUGAAGGGGACUAAAA |
| | Acinetobacter spp. 16S-23S | | CAAACAGUCGAUGUUACCUACA |
| | ribosomal RNA intergenic | | |
| | spacer | | |
| | CasM.26 crRNA targeting | 680 | GCCACCCCAAAAAUGAAGGGGACUAAAA |
| | Acinetobacter spp. 16S-23S | | CAAGUCGAUGUUACCUACAAGU |
| | ribosomal RNA intergenic | | |
| | spacer | | |
| | CasM.26 crRNA targeting | 681 | GCCACCCCAAAAAUGAAGGGGACUAAAA |
| | Acinetobacter spp. 16S-23S | | CAGCACCGUACAGCUUCAAUCU |
| | ribosomal RNA intergenic | | |
| | spacer | | |
| | CasM.26 crRNA targeting | 682 | GCCACCCCAAAAAUGAAGGGGACUAAAA |
| | Acinetobacter spp. 16S-23S | | CAUGCACUUAAGCACCGUACAG |
| | ribosomal RNA intergenic | | |
| | spacer | | |
| | CasM.26 crRNA targeting | 683 | GCCACCCCAAAAAUGAAGGGGACUAAAA |
| | Acinetobacter spp. 16S-23S | | CAUUAAGCACCGUACAGCUUCA |
| | ribosomal RNA intergenic | | |
| | spacer | | |
| | CasM.26 crRNA targeting | 684 | GCCACCCCAAAAAUGAAGGGGACUAAAA |
| | Acinetobacter spp. 16S-23S | | CAAAACAGUCGAUGUUACCUAC |
| | ribosomal RNA intergenic | | |
| | spacer | | |
| | CasM.26 crRNA targeting | 685 | GCCACCCCAAAAAUGAAGGGGACUAAAA |
| | Acinetobacter spp. 16S-23S | | CAUUCACAUACCAAAACGCUUG |
| | ribosomal RNA intergenic | | |
| | spacer | | |
| | CasM.26 crRNA targeting | 686 | GCCACCCCAAAAAUGAAGGGGACUAAAA |
| | Acinetobacter spp. 16S-23S | | CAACAACCCCAAACAGUCGAUG |
| | ribosomal RNA intergenic | | |
| | spacer | | |
| | CasM.26 crRNA targeting | 687 | GCCACCCCAAAAAUGAAGGGGACUAAAA |
| | Acinetobacter spp. 16S-23S | | CACGUGCGAUUCUUCAUCGCUA |
| | ribosomal RNA intergenic | | |
| | spacer | | |
| | CasM.26 crRNA targeting | 688 | GCCACCCCAAAAAUGAAGGGGACUAAAA |
| | Acinetobacter spp. 16S-23S | | CAUGUCCGUGCGAUUCUUCAUC |
| | ribosomal RNA intergenic | | |
| | spacer | | |
| | CasM.26 crRNA targeting | 689 | GCCACCCCAAAAAUGAAGGGGACUAAAA |
| | Acinetobacter spp. 16S-23S | | CAGAUUCUUCAUCGCUACUAUC |
| | ribosomal RNA intergenic | | |
| | spacer | | |
| | CasM.26 crRNA targeting | 687 | GCCACCCCAAAAAUGAAGGGGACUAAAA |
| | Acinetobacter spp. 16S-23S | | CACGUGCGAUUCUUCAUCGCUA |
| | ribosomal RNA intergenic | | |
| | spacer | | |
| | CasM.26 crRNA targeting | 690 | GCCACCCCAAAAAUGAAGGGGACUAAAA |
| | Acinetobacter spp. 16S-23S | | CAAGUGUUGUCCGUGCGAUUCU |
| | ribosomal RNA intergenic | | |
| | spacer | | |
| | CasM.26 crRNA targeting | 691 | GCCACCCCAAAAAUGAAGGGGACUAAAA |
| | Acinetobacter spp. 16S-23S | | CAUACAAGUGAGUGUUGUCCGU |
| | ribosomal RNA intergenic | | |
| | spacer | | |
| | CasM.26 crRNA targeting | 692 | GCCACCCCAAAAAUGAAGGGGACUAAAA |
| | Acinetobacter spp. 16S-23S | | CAGUCCGUGCGAUUCUUCAUCG |
| | ribosomal RNA intergenic | | |
| | spacer | | |
| | CasM.26 crRNA targeting | 693 | GCCACCCCAAAAAUGAAGGGGACUAAAA |
| | Acinetobacter spp. 16S-23S | | CACUACAAGUGAGUGUUGUCCG |
| | ribosomal RNA intergenic | | |
| | spacer | | |
| | CasM.26 crRNA targeting | 694 | GCCACCCCAAAAAUGAAGGGGACUAAAA |
| | Acinetobacter spp. 16S-23S | | CAGUCGUCAACACCUACAAGUG |
| | ribosomal RNA intergenic | | |
| | spacer | | |
| | CasM.26 crRNA targeting | 695 | GCCACCCCAAAAAUGAAGGGGACUAAAA |
| | Acinetobacter spp. 16S-23S | | CAAAGUGAGUGUUGUCCGUGCG |
| | ribosomal RNA intergenic | | |
| | spacer | | |
| | CasM.26 crRNA targeting | 696 | GCCACCCCAAAAAUGAAGGGGACUAAAA |
| | Acinetobacter spp. 16S-23S | | CAUGCGAUUCUUCAUCGCUACU |
| | ribosomal RNA intergenic | | |
| | spacer | | |
| | CasM.26 crRNA targeting | 697 | GCCACCCCAAAAAUGAAGGGGACUAAAA |
| | Acinetobacter spp. 16S-23S | | CAUUGUCCGUGCGAUUCUUCAU |
| | ribosomal RNA intergenic | | |
| | spacer | | |
| | CasM.26 crRNA targeting | 698 | GCCACCCCAAAAAUGAAGGGGACUAAAA |
| | Acinetobacter spp. 16S-23S | | CACGACGUUACCUACAAGUUAG |
| | ribosomal RNA intergenic | | |
| | spacer | | |
| | CasM.26 crRNA targeting | 699 | GCCACCCCAAAAAUGAAGGGGACUAAAA |
| | Acinetobacter spp. 16S-23S | | CAACCCCAAACAGUCGACGUUA |
| | ribosomal RNA intergenic | | |
| | spacer | | |
| | CasM.26 crRNA targeting | 700 | GCCACCCCAAAAAUGAAGGGGACUAAAA |
| | Acinetobacter spp. 16S-23S | | CACAAACAGUCGACGUUACCUA |
| | ribosomal RNA intergenic | | |
| | spacer | | |
| | CasM.26 crRNA targeting | 701 | GCCACCCCAAAAAUGAAGGGGACUAAAA |
| | Acinetobacter spp. 16S-23S | | CAGACGUUACCUACAAGUUAGU |
| | ribosomal RNA intergenic | | |
| | spacer | | |
| | CasM.26 crRNA targeting | 702 | GCCACCCCAAAAAUGAAGGGGACUAAAA |
| | Acinetobacter spp. 16S-23S | | CACAGUCGACGUUACCUACAAG |
| | ribosomal RNA intergenic | | |
| | spacer | | |
| | CasM.26 crRNA targeting | 703 | GCCACCCCAAAAAUGAAGGGGACUAAAA |
| | Acinetobacter spp. 16S-23S | | CAUAUACCAAAACGCUUGAUUC |
| | ribosomal RNA intergenic | | |
| | spacer | | |
| | CasM.26 crRNA targeting | 704 | GCCACCCCAAAAAUGAAGGGGACUAAAA |
| | Acinetobacter spp. 16S-23S | | CACAGUCGUUAUUACCUACAAG |
| | ribosomal RNA intergenic | | |
| | spacer | | |
| | CasM.26 crRNA targeting | 705 | GCCACCCCAAAAAUGAAGGGGACUAAAA |
| | Acinetobacter spp. 16S-23S | | CACCCAACAGUCGUUAUUACCU |
| | ribosomal RNA intergenic | | |
| | spacer | | |
| | CasM.26 crRNA targeting | 706 | GCCACCCCAAAAAUGAAGGGGACUAAAA |
| | Acinetobacter spp. 16S-23S | | CACAAUCUCUUACAGUUUGAAG |
| | ribosomal RNA intergenic | | |
| | spacer | | |
| | CasM.26 crRNA targeting | 707 | GCCACCCCAAAAAUGAAGGGGACUAAAA |
| | Proteus mirabilis rpoD | | CAACGGCUUGGGUGGCGUAAUU |
| | CasM.26 crRNA targeting | 708 | GCCACCCCAAAAAUGAAGGGGACUAAAA |
| | Proteus mirabilis rpoD | | CAGUAAUUUACGCAACGCUUUU |
| | CasM.26 crRNA targeting | 709 | GCCACCCCAAAAAUGAAGGGGACUAAAA |
| | Proteus mirabilis rpoD | | CAGCAACGCUUUUGCUUCAAUC |
| | CasM.26 crRNA targeting | 710 | GCCACCCCAAAAAUGAAGGGGACUAAAA |
| | Proteus vulgaris 16S | | CACUUCACCCCAGUCAUGAAUC |
| | CasM.26 crRNA targeting | 711 | GCCACCCCAAAAAUGAAGGGGACUAAAA |
| | Proteus vulgaris 16S | | CAAGUCAUGAAUCACAAAGUGG |
| | CasM.26 crRNA targeting | 712 | GCCACCCCAAAAAUGAAGGGGACUAAAA |
| | Proteus vulgaris 16S | | CACGACUUCACCCCAGUCAUGA |
| | CasM.26 crRNA targeting A. | 713 | GCCACCCCAAAAAUGAAGGGGACUAAAA |
| | pitti gyrB | | CAUAUCCGCAUUUAAAGCAUUC |
| | CasM.26 crRNA targeting A. | 714 | GCCACCCCAAAAAUGAAGGGGACUAAAA |
| | pitti gyrB | | CAUAAAGCAUUCUCUUCAAUGA |
| | CasM.26 crRNA targeting A. | 715 | GCCACCCCAAAAAUGAAGGGGACUAAAA |
| | pitti gyrB | | CAAGCAAGAGAACAUACGAUCU |
| | CasM.26 crRNA targeting | 716 | GCCACCCCAAAAAUGAAGGGGACUAAAA |
| | Enterobacter nimipressuralis | | CACUACCUUGUUACGACUUCAC |
| | 16S | | |
| | CasM.26 crRNA targeting | 717 | GCCACCCCAAAAAUGAAGGGGACUAAAA |
| | Enterobacter nimipressuralis | | CACCUACGGCUACCUUGUUACG |
| | 16S | | |
| | CasM.26 crRNA targeting | 718 | GCCACCCCAAAAAUGAAGGGGACUAAAA |
| | Enterobacter nimipressuralis | | CAAGGUUCCCCUACGGCUACCU |
| | 16S | | |
| | CasM.26 crRNA targeting | 719 | GCCACCCCAAAAAUGAAGGGGACUAAAA |
| | Bacillus anthracis pagA | | CACCGUUGGUACUAGUAUCCCC |
| | CasM.26 crRNA targeting | 720 | GCCACCCCAAAAAUGAAGGGGACUAAAA |
| | Bacillus anthracis pagA | | CAUGAUCCCGUUGGUACUAGUA |
| | CasM.26 crRNA targeting | 721 | GCCACCCCAAAAAUGAAGGGGACUAAAA |
| | Bacillus anthracis pagA | | CAGGGAUUACUUAUAUAUAACG |
| | CasM.26 crRNA targeting | 722 | GCCACCCCAAAAAUGAAGGGGACUAAAA |
| | Bacillus anthracis capB | | CACCACUCCAUAUACAAUCCGA |
| | CasM.26 crRNA targeting | 723 | GCCACCCCAAAAAUGAAGGGGACUAAAA |
| | Bacillus anthracis capB | | CAUCGAUUAAUGGCUCAGCUGC |
| | CasM.26 crRNA targeting | 724 | GCCACCCCAAAAAUGAAGGGGACUAAAA |
| | Bacillus anthracis capB | | CAAAUCAUAUCGAUUAAUGGCU |
| | CasM.26 crRNA targeting | 725 | GCCACCCCAAAAAUGAAGGGGACUAAAA |
| | Brucella spp. 23S | | CAUAGUACCGGUAAGCUGCAUG |
| | CasM.26 crRNA targeting | 726 | GCCACCCCAAAAAUGAAGGGGACUAAAA |
| | Brucella spp. 23S | | CAUAAGCUGCAUGCGUUGCCGC |
| | CasM.26 crRNA targeting | 727 | GCCACCCCAAAAAUGAAGGGGACUAAAA |
| | Brucella spp. 23S | | CAGGGAAUACUCGUUUUCAGGU |
| | CasM.26 crRNA targeting | 728 | GCCACCCCAAAAAUGAAGGGGACUAAAA |
| | Brucella spp. bcsp31 | | CACUUACGCGCAACGAUAUGGA |
| | CasM.26 crRNA targeting | 729 | GCCACCCCAAAAAUGAAGGGGACUAAAA |
| | Brucella spp. bcsp31 | | CAAUCGUUUCCGGGUAAAGCGU |
| | CasM.26 crRNA targeting | 730 | GCCACCCCAAAAAUGAAGGGGACUAAAA |
| | Brucella spp. bcsp31 | | CAUUGCAUCCUUACGCGCAACG |
| | CasM.26 crRNA targeting | 731 | GCCACCCCAAAAAUGAAGGGGACUAAAA |
| | Brucella spp. omp2a | | CAAGUAGGGCAGUAGGGCAGUA |
| | CasM.26 crRNA targeting | 732 | GCCACCCCAAAAAUGAAGGGGACUAAAA |
| | Brucella spp. omp2a | | CAUCGCACUUUUGGCUCGAAAA |
| | CasM.26 crRNA targeting | 733 | GCCACCCCAAAAAUGAAGGGGACUAAAA |
| | Brucella spp. omp2a | | CAGGGCAGUAGGGCAGUAGGGC |
| | CasM.26 crRNA targeting | 734 | GCCACCCCAAAAAUGAAGGGGACUAAAA |
| | Coxiella burnetii IS110 | | CAAUUUAUCCGCGGUGUUAAUC |
| | CasM.26 crRNA targeting | 735 | GCCACCCCAAAAAUGAAGGGGACUAAAA |
| | Coxiella burnetii IS110 | | CAGAUUUAUCCGCGGUGUUAAU |
| | CasM.26 crRNA targeting | 736 | GCCACCCCAAAAAUGAAGGGGACUAAAA |
| | Coxiella burnetii IS110 | | CAUUAUCCGCGGUGUUAAUCCC |
| | CasM.26 crRNA targeting | 737 | GCCACCCCAAAAAUGAAGGGGACUAAAA |
| | Francisella tularensis 16S | | CAACGGCUACCAUUGUUACGAC |
| | CasM.26 crRNA targeting | 738 | GCCACCCCAAAAAUGAAGGGGACUAAAA |
| | Francisella tularensis 16S | | CACCGCAGGUUCCCUACGGCUA |
| | CasM.26 crRNA targeting | 739 | GCCACCCCAAAAAUGAAGGGGACUAAAA |
| | Francisella tularensis 16S | | CAGGUUCCCUACGGCUACCAUU |
| | CasM.26 crRNA targeting | 740 | GCCACCCCAAAAAUGAAGGGGACUAAAA |
| | Rickettsia spp. 16S | | CACCAGUCGCUAAUUUUACCGU |
| | CasM.26 crRNA targeting | 741 | GCCACCCCAAAAAUGAAGGGGACUAAAA |
| | Rickettsia spp. 16S | | CAAUUUUACCGUGGUUGGCUGC |
| | CasM.26 crRNA targeting | 742 | GCCACCCCAAAAAUGAAGGGGACUAAAA |
| | Rickettsia spp. 16S | | CACUAACUCCCAUGGCGUGACG |
| | CasM.26 crRNA targeting | 743 | GCCACCCCAAAAAUGAAGGGGACUAAAA |
| | Rickettsia spp. 23S | | CACAAUCGAGCUAUUAGUAUCG |
| | CasM.26 crRNA targeting | 744 | GCCACCCCAAAAAUGAAGGGGACUAAAA |
| | Rickettsia spp. 23S | | CAAGUAUCGGUUAGCUACACAC |
| | CasM.26 crRNA targeting | 745 | GCCACCCCAAAAAUGAAGGGGACUAAAA |
| | Rickettsia spp. 23S | | CAUUCCACGGCUCUAAUGGGGA |
| | CasM.26 crRNA targeting | 746 | GCCACCCCAAAAAUGAAGGGGACUAAAA |
| | Rickettsia spp. 782 - 17K genus | | CAAGGUUGACGGCAUGCAUUAC |
| | common antigen | | |
| | CasM.26 crRNA targeting | 747 | GCCACCCCAAAAAUGAAGGGGACUAAAA |
| | Rickettsia spp. 782 - 17K genus | | CAGACGGCAUGCAUUACCAUAU |
| | common antigen | | |
| | CasM.26 crRNA targeting | 748 | GCCACCCCAAAAAUGAAGGGGACUAAAA |
| | Rickettsia spp. 782 - 17K genus | | CACCAUCAGGUUGACGGCAUGC |
| | common antigen | | |
| | CasM.26 crRNA targeting | 749 | GCCACCCCAAAAAUGAAGGGGACUAAAA |
| | Rickettsia spp. 783 - 17K genus | | CAGGACCGUUACAGGCUUGUAA |
| | common antigen | | |
| | CasM.26 crRNA targeting | 750 | GCCACCCCAAAAAUGAAGGGGACUAAAA |
| | Rickettsia spp. 783 - 17K genus | | CAGUUGCAAGAGCUAUAAUCAU |
| | common antigen | | |
| | CasM.26 crRNA targeting | 751 | GCCACCCCAAAAAUGAAGGGGACUAAAA |
| | Rickettsia spp. 783 - 17K genus | | CAGGCUUGUAACAUAGAAGUUG |
| | common antigen | | |
| | CasM.26 crRNA targeting | 752 | GCCACCCCAAAAAUGAAGGGGACUAAAA |
| | Yersinia pestis pMT1 | | CAUGGUUAGAUACGGUUACGGU |
| | CasM.26 crRNA targeting | 753 | GCCACCCCAAAAAUGAAGGGGACUAAAA |
| | Yersinia pestis pMT1 | | CAACGGUUACGGUUACAGCAUC |
| | CasM.26 crRNA targeting | 754 | GCCACCCCAAAAAUGAAGGGGACUAAAA |
| | Yersinia pestis pMT1 | | CAGGAGGGCAUAAUAGCCCUCC |
| | CasM.26 crRNA targeting | 755 | GCCACCCCAAAAAUGAAGGGGACUAAAA |
| | Yersinia pestis pCD1 | | CAUACCAGACGUGUCAUCUAGC |
| | CasM.26 crRNA targeting | 756 | GCCACCCCAAAAAUGAAGGGGACUAAAA |
| | Yersinia pestis pCD1 | | CAGACGUUGCAUCACUGAAUCA |
| | CasM.26 crRNA targeting | 757 | GCCACCCCAAAAAUGAAGGGGACUAAAA |
| | Yersinia pestis pCD1 | | CAACGUGUCAUCUAGCAGACGU |
| | CasM.26 crRNA targeting | 758 | GCCACCCCAAAAAUGAAGGGGACUAAAA |
| | Yersinia pestis pPCP1 | | CAGACCCGCCGUCACAGUAUAA |
| | CasM.26 crRNA targeting | 759 | GCCACCCCAAAAAUGAAGGGGACUAAAA |
| | Yersinia pestis pPCP1 | | CAGAUAUUGCAGACCCGCCGUC |
| | CasM.26 crRNA targeting | 760 | GCCACCCCAAAAAUGAAGGGGACUAAAA |
| | Yersinia pestis pPCP1 | | CAAUCUCCGCCAAUAGAGACAG |
| | Cas12 Variant crRNA targeting | 761 | UAAUUUCUACUAAGUGUAGAUAUAAGC |
| | Acinetobacter spp. | | GUGGGGUCACAAGU |
| | ACIN_PAN_1_19 | | |
| | Cas12 Variant crRNA targeting | 762 | UAAUUUCUACUAAGUGUAGAUGUCAGAC |
| | Acinetobacter spp. | | CCACCAUGACUUU |
| | ACIN_PAN_1_24 | | |
| | Cas12 Variant crRNA targeting | 763 | UAAUUUCUACUAAGUGUAGAUACUGAA |
| | Acinetobacter spp. | | UCAAGCGUUUUGGU |
| | ACIN_PAN_1_108 | | |
| | Cas12 Variant crRNA targeting | 764 | UAAUUUCUACUAAGUGUAGAUCGAGGU |
| | A. pittii gyrB | | UGUCGAUAACGCAA |
| | Cas12 Variant crRNA targeting | 765 | UAAUUUCUACUAAGUGUAGAUAUCGCGC |
| | A. pittii gyrB | | UGGCCAAGUGCAU |
| | Cas12 Variant crRNA targeting | 766 | UAAUUUCUACUAAGUGUAGAUCAGUUG |
| | A. pittii gyrB | | UAAAUGCGCUUUCA |
| | Cas12 Variant crRNA targeting | 767 | UAAUUUCUACUAAGUGUAGAUGCUACUG |
| | A. calcoaceticus gyrB | | AUCUUAGCGGCGG |
| | Cas12 Variant crRNA targeting | 768 | UAAUUUCUACUAAGUGUAGAUAUACCUA |
| | A. calcoaceticus gyrB | | GAGAUAGUGGACG |
| | Cas12 Variant crRNA targeting | 769 | UAAUUUCUACUAAGUGUAGAUUAGUUA |
| | A. calcoaceticus gyrB | | AUACCUAGAGAUAG |
| | Cas12 Variant crRNA targeting | 770 | UAAUUUCUACUAAGUGUAGAUGCUACUU |
| | Bacillus anthracis pagA | | CCGCUGAUAAUCA |
| | Cas12 Variant crRNA targeting | 771 | UAAUUUCUACUAAGUGUAGAUUCAAGCA |
| | Bacillus anthracis pagA | | CCCAUGGUGGUUA |
| | Cas12 Variant crRNA targeting | 772 | UAAUUUCUACUAAGUGUAGAUCAAGUU |
| | Bacillus anthracis pagA | | GUACUGGACCGAUU |
| | Cas12 Variant crRNA targeting | 773 | UAAUUUCUACUAAGUGUAGAUACGACUC |
| | Bacillus anthracis capB | | UACUUAAGUCGAA |
| | Cas12 Variant crRNA targeting | 774 | UAAUUUCUACUAAGUGUAGAUCUGACGA |
| | Bacillus anthracis capB | | GGAGCAACCGAUU |
| | Cas12 Variant crRNA targeting | 775 | UAAUUUCUACUAAGUGUAGAUGUACAUC |
| | Bacillus anthracis capB | | UGCGCGAAUGAUA |
| | Cas12 Variant crRNA targeting | 776 | UAAUUUCUACUAAGUGUAGAUGGGUUA |
| | Francisella tularensis tul4 | | UUAUUGUAUGCUGU |
| | Cas12 Variant crRNA targeting | 777 | UAAUUUCUACUAAGUGUAGAUGAAGCU |
| | Francisella tularensis tul4 | | UGUAUCAUGGCACU |
| | Cas12 Variant crRNA targeting | 778 | UAAUUUCUACUAAGUGUAGAUAGCCUGC |
| | Francisella tularensis tul4 | | CAUUGUAAUCUUA |
| | Cas12 Variant crRNA targeting | 779 | UAAUUUCUACUAAGUGUAGAUUAAAGCC |
| | Francisella tularensis fopA | | AUAAAAAACUACA |
| | Cas12 Variant crRNA targeting | 780 | UAAUUUCUACUAAGUGUAGAUAAUCAU |
| | Francisella tularensis fopA | | GUAGAAUACUUGAG |
| | Cas12 Variant crRNA targeting | 781 | UAAUUUCUACUAAGUGUAGAUGUGAACC |
| | Francisella tularensis fopA | | UGAAUGCCCUAAU |
| | Cas12 Variant crRNA targeting | 782 | UAAUUUCUACUAAGUGUAGAUGUACGCU |
| | Yersinia pestis pMT1 | | UACUCUUGGCGGC |
| | Cas12 Variant crRNA targeting | 783 | UAAUUUCUACUAAGUGUAGAUGUUCCGU |
| | Yersinia pestis pMT1 | | UAUCGCCAUUGCA |
| | Cas12 Variant crRNA targeting | 784 | UAAUUUCUACUAAGUGUAGAUUACAGA |
| | Yersinia pestis pMT1 | | UGCCGCGGGUGAUC |
| | Cas12 Variant crRNA targeting | 785 | UAAUUUCUACUAAGUGUAGAUAAUUGC |
| | Yersinia pestis pCD1 | | UCAAGAAAAUCCUA |
| | Cas12 Variant crRNA targeting | 786 | UAAUUUCUACUAAGUGUAGAUAAUACAC |
| | Yersinia pestis pCD1 | | AAUGGGAAUUGCG |
| | Cas12 Variant crRNA targeting | 787 | UAAUUUCUACUAAGUGUAGAUUUGCCAA |
| | Yersinia pestis pCD1 | | UAGAGUAAUUACU |
| | Cas12 Variant crRNA targeting | 788 | UAAUUUCUACUAAGUGUAGAUUAACUA |
| | Yersinia pestis pPCP1 | | UUCUGUCCGGGAGU |
| | Cas12 Variant crRNA targeting | 789 | UAAUUUCUACUAAGUGUAGAUCCGGGUC |
| | Yersinia pestis pPCP1 | | AGGUAAUAUGGAU |
| | Cas12 Variant crRNA targeting | 790 | UAAUUUCUACUAAGUGUAGAUUCCGGGA |
| | Yersinia pestis pPCP1 | | GUGCUAAUGCAGC |
| | Cas12 Variant crRNA targeting | 791 | UAAUUUCUACUAAGUGUAGAUACCGCAG |
| | Coxiella burnetii com1 | | GCGUGGCGAUAGC |
| | Cas12 Variant crRNA targeting | 792 | UAAUUUCUACUAAGUGUAGAUAUUUUU |
| | Coxiella burnetii com1 | | GGCGGCCAAUCGCA |
| | Cas12 Variant crRNA targeting | 793 | UAAUUUCUACUAAGUGUAGAUUUAGCCG |
| | Coxiella burnetii com1 | | GAACCUUGACCGC |
| | Cas12 Variant crRNA targeting | 794 | UAAUUUCUACUAAGUGUAGAUCCGCGGU |
| | Coxiella burnetii IS110 | | GUUAAUCCCACCG |
| | Cas12 Variant crRNA targeting | 795 | UAAUUUCUACUAAGUGUAGAUAGACGU |
| | Coxiella burnetii IS110 | | UCAAGCGCUGUUAA |
| | Cas12 Variant crRNA targeting | 796 | UAAUUUCUACUAAGUGUAGAUCAAAAG |
| | Coxiella burnetii IS110 | | AUGUUUUCCAGCUG |
| | Cas12 Variant crRNA targeting | 797 | UAAUUUCUACUAAGUGUAGAUGCACCUA |
| | Rickettsia spp. 782 - 17K genus | | GCGGUAGUAACAU |
| | common antigen | | |
| | Cas12 Variant crRNA targeting | 798 | UAAUUUCUACUAAGUGUAGAUGUACACU |
| | Rickettsia spp. 782 - 17K genus | | UCUUGGUGGCGCA |
| | common antigen | | |
| | Cas12 Variant crRNA targeting | 799 | UAAUUUCUACUAAGUGUAGAUCAGGUU |
| | Rickettsia spp. 782 - 17K genus | | GACGGCAUGCAUUA |
| | common antigen | | |
| | Cas12 Variant crRNA targeting | 800 | UAAUUUCUACUAAGUGUAGAUUCAUACC |
| | Rickettsia spp. 783 - 17K genus | | GCCCGGACCGUUA |
| | common antigen | | |
| | Cas12 Variant crRNA targeting | 801 | UAAUUUCUACUAAGUGUAGAUCAAGCCU |
| | Rickettsia spp. 783 - 17K genus | | GUAACGGUCCGGG |
| | common antigen | | |
| | Cas12 Variant crRNA targeting | 802 | UAAUUUCUACUAAGUGUAGAUCAGAACC |
| | Rickettsia spp. 783 - 17K genus | | UCAUUAUAAUUGA |
| | common antigen | | |
| | Cas12 Variant crRNA targeting | 803 | UAAUUUCUACUAAGUGUAGAUAUGCGA |
| | Brucella spp. bcsp31 | | UCAAGUCGGGCGCU |
| | Cas12 Variant crRNA targeting | 804 | UAAUUUCUACUAAGUGUAGAUUAUCGU |
| | Brucella spp. bcsp31 | | UGCGCGUAAGGAUG |
| | Cas12 Variant crRNA targeting | 805 | UAAUUUCUACUAAGUGUAGAUACGCGCA |
| | Brucella spp. bcsp31 | | ACGAUAUGGAUCG |
| | Cas12 Variant crRNA targeting | 806 | UAAUUUCUACUAAGUGUAGAUUUCACAC |
| | Brucella spp. omp2a | | UUUUCGGGAUGCG |
| | Cas12 Variant crRNA targeting | 807 | UAAUUUCUACUAAGUGUAGAUACUGCCC |
| | Brucella spp. omp2a | | UACUGCCCUACUG |
| | Cas12 Variant crRNA targeting | 808 | UAAUUUCUACUAAGUGUAGAUCACUUUU |
| | Brucella spp. omp2a | | GGCUCGAAAAUGC |
| | CasM.26 crRNA targeting | 809 | GCCACCCCAAAAAUGAAGGGGACUAAAA |
| | Proteus vulgaris rpoD | | CACUACGUAAUACUUCAGAACG |
| | CasM.26 crRNA targeting | 810 | GCCACCCCAAAAAUGAAGGGGACUAAAA |
| | Proteus vulgaris rpoD | | CAGUCAAGGAAGCUACGUAAUA |
| | CasM.26 crRNA targeting | 811 | GCCACCCCAAAAAUGAAGGGGACUAAAA |
| | Proteus vulgaris rpoD | | CAGUAAUACUUCAGAACGGCUU |
| | CasM.26 crRNA targeting | 812 | GCCACCCCAAAAAUGAAGGGGACUAAAA |
| | Proteus penneri rpoD | | CAAGCUGAAGCUACUUGUUACU |
| | CasM.26 crRNA targeting | 813 | GCCACCCCAAAAAUGAAGGGGACUAAAA |
| | Proteus penneri rpoD | | CAAGCUACUUGUUACUAAAGGU |
| | CasM.26 crRNA targeting | 814 | GCCACCCCAAAAAUGAAGGGGACUAAAA |
| | Proteus penneri rpoD | | CAUUGUUACUAAAGGUAAGGAG |
| | CasM.26 crRNA targeting | 815 | GCCACCCCAAAAAUGAAGGGGACUAAAA |
| | Proteus hauseri rpoD | | CAUCUAGGAAGCUACGUAAGAC |
| | CasM.26 crRNA targeting | 816 | GCCACCCCAAAAAUGAAGGGGACUAAAA |
| | Proteus hauseri rpoD | | CAGCGGCUAGGGUGGCGUAACU |
| | CasM.26 crRNA targeting | 817 | GCCACCCCAAAAAUGAAGGGGACUAAAA |
| | Proteus hauseri rpoD | | CAGCGUAACUUACGCAGUGCCU |
| | CasM.26 crRNA targeting A. | 818 | GCCACCCCAAAAAUGAAGGGGACUAAAA |
| | baumannii gyrB | | CACGCCCAUUAAGCAUGAGAAC |
| | CasM.26 crRNA targeting A. | 819 | GCCACCCCAAAAAUGAAGGGGACUAAAA |
| | baumannii gyrB | | CAGCAUGAGAACAUGCGAUCAG |
| | CasM.26 crRNA targeting A. | 820 | GCCACCCCAAAAAUGAAGGGGACUAAAA |
| | baumannii gyrB | | CAGCGAUCAGCUUCAAUUGCAU |
| | CasM.26 crRNA targeting A. | 821 | GCCACCCCAAAAAUGAAGGGGACUAAAA |
| | nosocomialis gyrB | | CAAGCAUGAGAACAUACGAUCU |
| | CasM.26 crRNA targeting A. | 822 | GCCACCCCAAAAAUGAAGGGGACUAAAA |
| | nosocomialis gyrB | | CAGUCAUCACCCAUUAAGCAUG |
| | CasM.26 crRNA targeting A. | 823 | GCCACCCCAAAAAUGAAGGGGACUAAAA |
| | nosocomialis gyrB | | CACACCCAUUAAGCAUGAGAAC |
| | CasM.26 crRNA targeting | 824 | GCCACCCCAAAAAUGAAGGGGACUAAAA |
| | Enterobacter cloacae dnaJ | | CACUUUCCUGCAGCUCUUUUAA |
| | CasM.26 crRNA targeting | 825 | GCCACCCCAAAAAUGAAGGGGACUAAAA |
| | Enterobacter cloacae dnaJ | | CAAGCUCUUUUAACAGCUGCUU |
| | CasM.26 crRNA targeting | 826 | GCCACCCCAAAAAUGAAGGGGACUAAAA |
| | Enterobacter cloacae dnaJ | | CAGAAGAAGCUUUUGGAGCGUG |
| | CasM.26 crRNA targeting | 827 | GCCACCCCAAAAAUGAAGGGGACUAAAA |
| | Enterobacter asburiae pyrG | | CAAUGCCCGUCACGUGGCGUUG |
| | CasM.26 crRNA targeting | 828 | GCCACCCCAAAAAUGAAGGGGACUAAAA |
| | Enterobacter asburiae pyrG | | CAGGUAGUCGCUGGCGGCCUUC |
| | CasM.26 crRNA targeting | 829 | GCCACCCCAAAAAUGAAGGGGACUAAAA |
| | Enterobacter asburiae pyrG | | CAUGCAAACAGCGGAUGCCCGU |
| | CasM.26 crRNA targeting | 830 | GCCACCCCAAAAAUGAAGGGGACUAAAA |
| | Enterobacter asburiae dnaJ | | CAAGCUCUCCUGCAGUUCUUUC |
| | CasM.26 crRNA targeting | 831 | GCCACCCCAAAAAUGAAGGGGACUAAAA |
| | Enterobacter asburiae dnaJ | | CAGCCAAAGCUCUCCUGCAGUU |
| | CasM.26 crRNA targeting | 832 | GCCACCCCAAAAAUGAAGGGGACUAAAA |
| | Enterobacter asburiae dnaJ | | CACUGCAGUUCUUUCAGCAGCU |
| | CasM.26 crRNA targeting | 829 | GCCACCCCAAAAAUGAAGGGGACUAAAA |
| | Enterobacter hormaechei pyrG | | CAUGCAAACAGCGGAUGCCCGU |
| | CasM.26 crRNA targeting | 833 | GCCACCCCAAAAAUGAAGGGGACUAAAA |
| | Enterobacter hormaechei pyrG | | CAAUGCCCGUCACGCGGCGUUG |
| | CasM.26 crRNA targeting | 834 | GCCACCCCAAAAAUGAAGGGGACUAAAA |
| | Enterobacter hormaechei pyrG | | CAGUGGAAUUGGCAGGCCACGA |
| | CasM.26 crRNA targeting | 835 | GCCACCCCAAAAAUGAAGGGGACUAAAA |
| | Enterobacter hormaechei dnaJ | | CAGGGUUAAAUCGUCGAAGAAU |
| | CasM.26 crRNA targeting | 836 | GCCACCCCAAAAAUGAAGGGGACUAAAA |
| | Enterobacter hormaechei dnaJ | | CAUUUCCUGAAGCUCUUUCAGC |
| | CasM.26 crRNA targeting | 837 | GCCACCCCAAAAAUGAAGGGGACUAAAA |
| | Enterobacter hormaechei dnaJ | | CACGUCGAAGAAUUUUUUGACG |
| | CasM.26 crRNA targeting | 829 | GCCACCCCAAAAAUGAAGGGGACUAAAA |
| | Enterobacter kobei pyrG | | CAUGCAAACAGCGGAUGCCCGU |
| | CasM.26 crRNA targeting | 833 | GCCACCCCAAAAAUGAAGGGGACUAAAA |
| | Enterobacter kobei pyrG | | CAAUGCCCGUCACGCGGCGUUG |
| | CasM.26 crRNA targeting | 838 | GCCACCCCAAAAAUGAAGGGGACUAAAA |
| | Enterobacter kobei pyrG | | CAACUGGCAGGCAACAAACCAU |
| | CasM.26 crRNA targeting | 839 | GCCACCCCAAAAAUGAAGGGGACUAAAA |
| | Enterobacter kobei dnaJ | | CAGGGUCAAGUCGUCGAAGAAU |
| | CasM.26 crRNA targeting | 840 | GCCACCCCAAAAAUGAAGGGGACUAAAA |
| | Enterobacter kobei dnaJ | | CAAGUCGUCGAAGAAUUUUUUG |
| | CasM.26 crRNA targeting | 841 | GCCACCCCAAAAAUGAAGGGGACUAAAA |
| | Enterobacter kobei dnaJ | | CAUGCAGCUCUUUCAGUAGCUG |
| | CasM.26 crRNA targeting | 827 | GCCACCCCAAAAAUGAAGGGGACUAAAA |
| | Enterobacter ludwigii pyrG | | CAAUGCCCGUCACGUGGCGUUG |
| | CasM.26 crRNA targeting | 829 | GCCACCCCAAAAAUGAAGGGGACUAAAA |
| | Enterobacter ludwigii pyrG | | CAUGCAAACAGCGGAUGCCCGU |
| | CasM.26 crRNA targeting | 842 | GCCACCCCAAAAAUGAAGGGGACUAAAA |
| | Enterobacter ludwigii pyrG | | CAAAUCGCUGGCGGCUUUCACA |
| | CasM.26 crRNA targeting | 843 | GCCACCCCAAAAAUGAAGGGGACUAAAA |
| | Enterobacter ludwigii dnaJ | | CAAACUUUCCUGCAGCUCUUUC |
| | CasM.26 crRNA targeting | 844 | GCCACCCCAAAAAUGAAGGGGACUAAAA |
| | Enterobacter ludwigii dnaJ | | CAGGCCGCCAAAACUUUCCUGC |
| | CasM.26 crRNA targeting | 845 | GCCACCCCAAAAAUGAAGGGGACUAAAA |
| | Enterobacter ludwigii dnaJ | | CAUGCAGCUCUUUCAGCAGCUG |
| | CasM.26 crRNA targeting | 846 | GCCACCCCAAAAAUGAAGGGGACUAAAA |
| | Enterobacter mori pyrG | | CAAUUGGCAGGCAACGAACCAU |
| | CasM.26 crRNA targeting | 847 | GCCACCCCAAAAAUGAAGGGGACUAAAA |
| | Enterobacter mori pyrG | | CACCCGUCGCGCGGCGUAGAAG |
| | CasM.26 crRNA targeting | 848 | GCCACCCCAAAAAUGAAGGGGACUAAAA |
| | Enterobacter mori pyrG | | CAAGUCGCUGGCGGCCUUCACA |
| | CasM.26 crRNA targeting | 849 | GCCACCCCAAAAAUGAAGGGGACUAAAA |
| | Enterobacter mori dnaJ | | CAACCAAAGCUUUCCUGCAGCU |
| | CasM.26 crRNA targeting | 850 | GCCACCCCAAAAAUGAAGGGGACUAAAA |
| | Enterobacter mori dnaJ | | CAUUUCUCACCCGUCGGGCCAC |
| | CasM.26 crRNA targeting | 851 | GCCACCCCAAAAAUGAAGGGGACUAAAA |
| | Enterobacter mori dnaJ | | CAAGCGCGUUAAAUCAUCGAAG |
| | Cas12 Variant crRNA targeting | 852 | UAAUUUCUACUAAGUGUAGAUGGGAUA |
| | A. baumannii gyrB | | CAGACGACGGUACC |
| | Cas12 Variant crRNA targeting | 853 | UAAUUUCUACUAAGUGUAGAUAGGUGG |
| | A. baumannii gyrB | | CUUACACGGCGUAG |
| | Cas12 Variant crRNA targeting | 854 | UAAUUUCUACUAAGUGUAGAUCAUUUA |
| | A. baumannii gyrB | | ACGAUUUACCGUGC |
| | Cas12 Variant crRNA targeting | 855 | UAAUUUCUACUAAGUGUAGAUCUGUGAC |
| | A. nosocomialis gyrB | | GAAAUUAUUGUCA |
| | Cas12 Variant crRNA targeting | 856 | UAAUUUCUACUAAGUGUAGAUUUCUUAC |
| | A. nosocomialis gyrB | | CAUUCUGCAUGCA |
| | Cas12 Variant crRNA targeting | 857 | UAAUUUCUACUAAGUGUAGAUGGGAUA |
| | A. nosocomialis gyrB | | CAGACGACGGUACU |
| | Cas12 Variant crRNA targeting | 858 | UAAUUUCUACUAAGUGUAGAUGUGGUG |
| | Enterobacter cloacae dnaJ | | GCCGCGGUCGUCAA |
| | Cas12 Variant crRNA targeting | 859 | UAAUUUCUACUAAGUGUAGAUCGCCGUC |
| | Enterobacter cloacae dnaJ | | CAGCAGGCGUGUC |
| | Cas12 Variant crRNA targeting | 860 | UAAUUUCUACUAAGUGUAGAUAUGACCA |
| | Enterobacter cloacae dnaJ | | GUACGGUCACGCA |
| | Cas12 Variant crRNA targeting | 861 | UAAUUUCUACUAAGUGUAGAUAGACCUG |
| | Enterobacter asburiae dnaJ | | UCCAACCUGUCAC |
| | Cas12 Variant crRNA targeting | 862 | UAAUUUCUACUAAGUGUAGAUCGCUGGA |
| | Enterobacter asburiae dnaJ | | AGAGUGUGACGUU |
| | Cas12 Variant crRNA targeting | 863 | UAAUUUCUACUAAGUGUAGAUACCUGCG |
| | Enterobacter asburiae dnaJ | | CUACAACAUGGAG |
| | Cas12 Variant crRNA targeting | 864 | UAAUUUCUACUAAGUGUAGAUAAGAAC |
| | Enterobacter asburiae pyrG | | GCAUUGUUGCGGGU |
| | Cas12 Variant crRNA targeting | 865 | UAAUUUCUACUAAGUGUAGAUUGGUGA |
| | Enterobacter asburiae pyrG | | CUACCUGGGUGCAA |
| | Cas12 Variant crRNA targeting | 866 | UAAUUUCUACUAAGUGUAGAUGCCACUA |
| | Enterobacter asburiae pyrG | | CGAGCGUUUCAUC |
| | Cas12 Variant crRNA targeting | 867 | UAAUUUCUACUAAGUGUAGAUUUAAAG |
| | Enterobacter hormaechei pyrG | | AGCGUGUCCUUGCC |
| | Cas12 Variant crRNA targeting | 866 | UAAUUUCUACUAAGUGUAGAUGCCACUA |
| | Enterobacter hormaechei pyrG | | CGAGCGUUUCAUC |
| | Cas12 Variant crRNA targeting | 868 | UAAUUUCUACUAAGUGUAGAUUGCAGG |
| | Enterobacter hormaechei pyrG | | UCAUCCCACACAUC |
| | Cas12 Variant crRNA targeting | 869 | UAAUUUCUACUAAGUGUAGAUACCGUAA |
| | Enterobacter hormaechei dnaJ | | UCAGGGUGACAAA |
| | Cas12 Variant crRNA targeting | 870 | UAAUUUCUACUAAGUGUAGAUCAACAUG |
| | Enterobacter hormaechei dnaJ | | GAGCUGACGCUUG |
| | Cas12 Variant crRNA targeting | 871 | UAAUUUCUACUAAGUGUAGAUAUGAUC |
| | Enterobacter hormaechei dnaJ | | AGUACGGUCACGCG |
| | Cas12 Variant crRNA targeting | 872 | UAAUUUCUACUAAGUGUAGAUAGGUUA |
| | Enterobacter kobei pyrG | | UCCCACACAUCACU |
| | Cas12 Variant crRNA targeting | 873 | UAAUUUCUACUAAGUGUAGAUCGACUGG |
| | Enterobacter kobei pyrG | | CCGCAUCUACUCC |
| | Cas12 Variant crRNA targeting | 874 | UAAUUUCUACUAAGUGUAGAUGUACAUC |
| | Enterobacter kobei pyrG | | AACGUCGAUCCUG |
| | Cas12 Variant crRNA targeting | 875 | UAAUUUCUACUAAGUGUAGAUCAGUACA |
| | Enterobacter kobei dnaJ | | GCAGGCCUGUCCU |
| | Cas12 Variant crRNA targeting | 876 | UAAUUUCUACUAAGUGUAGAUCGGUGUC |
| | Enterobacter kobei dnaJ | | ACCAAAGAGAUCC |
| | Cas12 Variant crRNA targeting | 877 | UAAUUUCUACUAAGUGUAGAUAUUUCA |
| | Enterobacter kobei dnaJ | | GCGACAUCUUCGGC |
| | Cas12 Variant crRNA targeting | 878 | UAAUUUCUACUAAGUGUAGAUUGGUGA |
| | Enterobacter ludwigii pyrG | | UUAUCUUGGUGCUA |
| | Cas12 Variant crRNA targeting | 879 | UAAUUUCUACUAAGUGUAGAUUUGCGG |
| | Enterobacter ludwigii pyrG | | GCGGCGAAGGCCAC |
| | Cas12 Variant crRNA targeting | 880 | UAAUUUCUACUAAGUGUAGAUUCCGCAC |
| | Enterobacter ludwigii pyrG | | UAAAAUGACCCGU |
| | Cas12 Variant crRNA targeting | 881 | UAAUUUCUACUAAGUGUAGAUACCUGCG |
| | Enterobacter ludwigii dnaJ | | CUACAACAUGGAU |
| | Cas12 Variant crRNA targeting | 882 | UAAUUUCUACUAAGUGUAGAUCGAACAA |
| | Enterobacter ludwigii dnaJ | | GGCGGCAUGGGCG |
| | Cas12 Variant crRNA targeting | 883 | UAAUUUCUACUAAGUGUAGAUCGGCGAC |
| | Enterobacter ludwigii dnaJ | | AUUUUUGGCGGCG |
| | Cas12 Variant crRNA targeting | 884 | UAAUUUCUACUAAGUGUAGAUAUUGGC |
| | Enterobacter mori pyrG | | AGGCAACGAACCAU |
| | Cas12 Variant crRNA targeting | 885 | UAAUUUCUACUAAGUGUAGAUCCCGUCG |
| | Enterobacter mori pyrG | | CGCGGCGUAGAAG |
| | Cas12 Variant crRNA targeting | 886 | UAAUUUCUACUAAGUGUAGAUAGUCGCU |
| | Enterobacter mori pyrG | | GGCGGCCUUCACA |
| | Cas12 Variant crRNA targeting | 887 | UAAUUUCUACUAAGUGUAGAUACCAAAG |
| | Enterobacter mori dnaJ | | CUUUCCUGCAGCU |
| | Cas12 Variant crRNA targeting | 888 | UAAUUUCUACUAAGUGUAGAUAGCGCGU |
| | Enterobacter mori dnaJ | | UAAAUCAUCGAAG |
| | Cas12 Variant crRNA targeting | 889 | UAAUUUCUACUAAGUGUAGAUUUUCUCA |
| | Enterobacter mori dnaJ | | CCCGUCGGGCCAC |
| | CasM.26 crRNA targeting | 890 | GCCACCCCAAAAAUGAAGGGGACUAAAA |
| | Coronavirus 229E | | CAGCUCGUCAAUGCGAUCUUUG |
| | CasM.26 crRNA targeting | 891 | GCCACCCCAAAAAUGAAGGGGACUAAAA |
| | Coronavirus HKU1 | | CAAAAUUUCUUGUAAAGAAGAA |
| | CasM.26 crRNA targeting | 892 | GCCACCCCAAAAAUGAAGGGGACUAAAA |
| | Coronavirus OC43 | | CAGAGUGCCUUAUCUCGACUUU |
| | CasM.26 crRNA targeting | 893 | GCCACCCCAAAAAUGAAGGGGACUAAAA |
| | hMPV | | CACAUCACAUCUUUCCAUGUUA |
| | CasM.26 crRNA targeting | 894 | GCCACCCCAAAAAUGAAGGGGACUAAAA |
| | Rhinovirus EN71 | | CAUAGUCGGUUCCGCUGCAGAG |
| | CasM.26 crRNA targeting | 895 | GCCACCCCAAAAAUGAAGGGGACUAAAA |
| | Rhinovirus ENA | | CAACGGACACCCAAAGUAGUCG |
| | CasM.26 crRNA targeting | 896 | GCCACCCCAAAAAUGAAGGGGACUAAAA |
| | Rhinovirus ENB | | CACCAAAGUAGUCGGUUCCGCU |
| | CasM.26 crRNA targeting | 897 | GCCACCCCAAAAAUGAAGGGGACUAAAA |
| | Rhinovirus ENC | | CAAGGAAACACGGACACCCAAA |
| | CasM.26 crRNA targeting | 895 | GCCACCCCAAAAAUGAAGGGGACUAAAA |
| | Rhinovirus END | | CAACGGACACCCAAAGUAGUCG |
| | CasM.26 crRNA targeting | 898 | GCCACCCCAAAAAUGAAGGGGACUAAAA |
| | Influenza A | | CAUGUUGACGAUGGUCAUUUUG |
| | CasM.26 crRNA targeting | 899 | GCCACCCCAAAAAUGAAGGGGACUAAAA |
| | Influenza B | | CAUUGAAGUGUACAGCAGGCAG |
| | CasM.26 crRNA targeting RSV | 900 | GCCACCCCAAAAAUGAAGGGGACUAAAA |
| | | | CAGACUUGAGAUAUUGAUGCAU |
| | CasM.26 crRNA targeting | 901 | GCCACCCCAAAAAUGAAGGGGACUAAAA |
| | Parainfluenza virus HPIV1 | | CAACAAGUAGUGCUGGUCUAAG |
| | CasM.26 crRNA targeting | 902 | GCCACCCCAAAAAUGAAGGGGACUAAAA |
| | Parainfluenza virus HPIV2 | | CAACAAGAUGGCCUACAUUGGC |
| | CasM.26 crRNA targeting | 903 | GCCACCCCAAAAAUGAAGGGGACUAAAA |
| | Parainfluenza virus HPIV3 | | CAGAAAAACUUAGGAUUAAAGA |
| | CasM.26 crRNA targeting | 904 | GCCACCCCAAAAAUGAAGGGGACUAAAA |
| | Parainfluenza virus HPIV4a | | CAUUAGCUGCUUACGAACAGUU |
| | CasM.26 crRNA targeting | 905 | GCCACCCCAAAAAUGAAGGGGACUAAAA |
| | Parainfluenza virus HPIV4b | | CAUAGACCCACAAGAUUAUACA |
| | CasM.26 crRNA targeting | 906 | GCCACCCCAAAAAUGAAGGGGACUAAAA |
| | Nocardia 16S | | CAGACUCCAACUUCACGGGGUC |
| | CasM.26 crRNA targeting | 907 | GCCACCCCAAAAAUGAAGGGGACUAAAA |
| | Nocardia 16S | | CAAACUUCACGGGGUCGAGUUG |
| | CasM.26 crRNA targeting | 908 | GCCACCCCAAAAAUGAAGGGGACUAAAA |
| | Nocardia 16S | | CAUAGCGACUCCAACUUCACGG |
| | CasM.26 crRNA targeting | 909 | GCCACCCCAAAAAUGAAGGGGACUAAAA |
| | Nocardia hsp65 | | CAUCCAUGGCCUCGGCGAUCAG |
| | CasM.26 crRNA targeting | 910 | GCCACCCCAAAAAUGAAGGGGACUAAAA |
| | Nocardia hsp65 | | CAUGUCCAUGGCCUCGGCGAUC |
| | CasM.26 crRNA targeting | 911 | GCCACCCCAAAAAUGAAGGGGACUAAAA |
| | Nocardia hsp65 | | CAGAUCAGCUCACCGAUGGACG |
| | CasM.26 crRNA targeting | 912 | GCCACCCCAAAAAUGAAGGGGACUAAAA |
| | Nocardia gyrB | | CAUAGGGGACGGUGUCGUCGUU |
| | CasM.26 crRNA targeting | 913 | GCCACCCCAAAAAUGAAGGGGACUAAAA |
| | Nocardia gyrB | | CACGGCUGGAGAAGAUCUUCCC |
| | CasM.26 crRNA targeting | 914 | GCCACCCCAAAAAUGAAGGGGACUAAAA |
| | Nocardia gyrB | | CAGGCGAAGAAGCCGUAGUAGC |
| | CasM.26 crRNA targeting | 915 | GCCACCCCAAAAAUGAAGGGGACUAAAA |
| | Nocardia secA1 | | CAAGGAUGGAGUCGACCUCGUC |
| | CasM.26 crRNA targeting | 916 | GCCACCCCAAAAAUGAAGGGGACUAAAA |
| | Nocardia secA1 | | CAGAUGUCGACCUCGUAGUGCA |
| | CasM.26 crRNA targeting | 917 | GCCACCCCAAAAAUGAAGGGGACUAAAA |
| | Nocardia secA1 | | CAUCGACCUCGUCGACCACGGC |
| | CasM.26 crRNA targeting | 918 | GCCACCCCAAAAAUGAAGGGGACUAAAA |
| | Nocardia sodA | | CAUGCUCCCACAUGUCGACCUG |
| | CasM.26 crRNA targeting | 919 | GCCACCCCAAAAAUGAAGGGGACUAAAA |
| | Nocardia sodA | | CAGAAGGUCAGCAGCUUCUGGC |
| | CasM.26 crRNA targeting | 920 | GCCACCCCAAAAAUGAAGGGGACUAAAA |
| | Nocardia sodA | | CAGCUGGAAGGUCAGCAGCUUC |
| | CasM.26 crRNA targeting | 921 | GCCACCCCAAAAAUGAAGGGGACUAAAA |
| | Cryptococcus 18S | | CACGCCAAUCCGGAGAUCUCAC |
| | CasM.26 crRNA targeting | 922 | GCCACCCCAAAAAUGAAGGGGACUAAAA |
| | Cryptococcus 18S | | CAUCAACGCGAGCUGGUGACUC |
| | CasM.26 crRNA targeting | 923 | GCCACCCCAAAAAUGAAGGGGACUAAAA |
| | Cryptococcus 18S | | CAGACUCACGCUUACUAGGUAU |
| | CasM.26 crRNA targeting | 924 | GCCACCCCAAAAAUGAAGGGGACUAAAA |
| | Cryptococcus URA5 | | CACUCGUCCCGGGAUUGAACCG |
| | CasM.26 crRNA targeting | 925 | GCCACCCCAAAAAUGAAGGGGACUAAAA |
| | Cryptococcus URA5 | | CACGAAACGGCUAUGCCACCGA |
| | CasM.26 crRNA targeting | 926 | GCCACCCCAAAAAUGAAGGGGACUAAAA |
| | Cryptococcus URA5 | | CAUCGGUGGCAUAGCCGUUUCG |
| | CasM.26 crRNA targeting | 927 | GCCACCCCAAAAAUGAAGGGGACUAAAA |
| | Cryptococcus ITS | | CAGCGAAACUUAUUACGCCGGG |
| | CasM.26 crRNA targeting | 928 | GCCACCCCAAAAAUGAAGGGGACUAAAA |
| | Cryptococcus ITS | | CAACUUAUUACGCCGGGCUGAC |
| | CasM.26 crRNA targeting | 929 | GCCACCCCAAAAAUGAAGGGGACUAAAA |
| | Cryptococcus ITS | | CAUAAGGCGAGCCGACGUCCUU |
| | CasM.26 crRNA targeting | 930 | GCCACCCCAAAAAUGAAGGGGACUAAAA |
| | Cryptococcus 28S | | CAGACGGGUCGUUUAAAGCCAU |
| | CasM.26 crRNA targeting | 931 | GCCACCCCAAAAAUGAAGGGGACUAAAA |
| | Cryptococcus 28S | | CAUCGAACGUGGGCGAACCCCG |
| | CasM.26 crRNA targeting | 932 | GCCACCCCAAAAAUGAAGGGGACUAAAA |
| | Cryptococcus 28S | | CAUGGGCGAACCCCGGCCAUAA |
| | CasM.26 crRNA targeting | 933 | GCCACCCCAAAAAUGAAGGGGACUAAAA |
| | Cryptococcus CTX1 | | CAGGUCAACCUCGCAUAUUCGA |
| | CasM.26 crRNA targeting | 934 | GCCACCCCAAAAAUGAAGGGGACUAAAA |
| | Cryptococcus CTX1 | | CACGCAUAUUCGAGGUACAACC |
| | CasM.26 crRNA targeting | 935 | GCCACCCCAAAAAUGAAGGGGACUAAAA |
| | Cryptococcus CTX1 | | CAACUGUCCGUUCAACGCAAUU |
| | CasM.26 crRNA targeting | 936 | GCCACCCCAAAAAUGAAGGGGACUAAAA |
| | Actinomyces 16S | | CAAUCACCCCACCAACAAGCUG |
| | CasM.26 crRNA targeting | 937 | GCCACCCCAAAAAUGAAGGGGACUAAAA |
| | Actinomyces 16S | | CAGCCAUCACCCCACCAACAAG |
| | CasM.26 crRNA targeting | 938 | GCCACCCCAAAAAUGAAGGGGACUAAAA |
| | Actinomyces 16S | | CAACCUAGUGCCCAACGUUUAC |
| | Cas12 Variant crRNA targeting | 939 | UAAUUUCUACUAAGUGUAGAUGACCGGC |
| | Nocardia 16S | | UUUAAGGGAUUCG |
| | Cas12 Variant crRNA targeting | 940 | UAAUUUCUACUAAGUGUAGAUCGACUUU |
| | Nocardia 16S | | CAUGACGUGACGG |
| | Cas12 Variant crRNA targeting | 941 | UAAUUUCUACUAAGUGUAGAUUCUGCGA |
| | Nocardia 16S | | UUACUAGCGACUC |
| | Cas12 Variant crRNA targeting | 942 | UAAUUUCUACUAAGUGUAGAUCGAUGG |
| | Nocardia hsp65 | | ACACACCAUCGUUG |
| | Cas12 Variant crRNA targeting | 943 | UAAUUUCUACUAAGUGUAGAUUCUCCUU |
| | Nocardia hsp65 | | GGCGAUGGACACA |
| | Cas12 Variant crRNA targeting | 944 | UAAUUUCUACUAAGUGUAGAUCGACCUU |
| | Nocardia hsp65 | | GUCCAUGGCCUCG |
| | Cas12 Variant crRNA targeting | 945 | UAAUUUCUACUAAGUGUAGAUCGAUGA |
| | Nocardia secA1 | | GGAUGGAGUCGACC |
| | Cas12 Variant crRNA targeting | 946 | UAAUUUCUACUAAGUGUAGAUGCCGUGG |
| | Nocardia secA1 | | UCGACGAGGUCGA |
| | Cas12 Variant crRNA targeting | 947 | UAAUUUCUACUAAGUGUAGAUGAUGUC |
| | Nocardia secA1 | | GACCUCGUAGUGCA |
| | Cas12 Variant crRNA targeting | 948 | UAAUUUCUACUAAGUGUAGAUCAGGUCG |
| | Nocardia sodA | | ACAUGUGGGAGCA |
| | Cas12 Variant crRNA targeting | 949 | UAAUUUCUACUAAGUGUAGAUAAGGUC |
| | Nocardia sodA | | AGCAGCUUCUGGCC |
| | Cas12 Variant crRNA targeting | 950 | UAAUUUCUACUAAGUGUAGAUGGCCAGA |
| | Nocardia sodA | | AGCUGCUGACCUU |
| | Cas12 Variant crRNA targeting | 951 | UAAUUUCUACUAAGUGUAGAUAAGCAG |
| | Cryptococcus 18S | | GCAAUCGCCCGAAU |
| | Cas12 Variant crRNA targeting | 952 | UAAUUUCUACUAAGUGUAGAUUAGUCG |
| | Cryptococcus 18S | | AACUUCAGGUCUGG |
| | Cas12 Variant crRNA targeting | 953 | UAAUUUCUACUAAGUGUAGAUGGGGCA |
| | Cryptococcus 18S | | UUGGUAUUCCGUUG |
| | Cas12 Variant crRNA targeting | 954 | UAAUUUCUACUAAGUGUAGAUUCGGUUC |
| | Cryptococcus URA5 | | AAUCCCGGGACGA |
| | Cas12 Variant crRNA targeting | 955 | UAAUUUCUACUAAGUGUAGAUCGGUGGC |
| | Cryptococcus URA5 | | AUAGCCGUUUCGU |
| | Cas12 Variant crRNA targeting | 956 | UAAUUUCUACUAAGUGUAGAUCGAAACG |
| | Cryptococcus URA5 | | GCUAUGCCACCGA |
| | Cas12 Variant crRNA targeting | 957 | UAAUUUCUACUAAGUGUAGAUGCGAAAC |
| | Cryptococcus ITS | | UUAUUACGCCGGG |
| | Cas12 Variant crRNA targeting | 958 | UAAUUUCUACUAAGUGUAGAUGCGUAA |
| | Cryptococcus ITS | | UAAGUUUCGCUGGG |
| | Cas12 Variant crRNA targeting | 959 | UAAUUUCUACUAAGUGUAGAUCACAUUU |
| | Cryptococcus ITS | | AAGGCGAGCCGAC |
| | Cas12 Variant crRNA targeting | 960 | UAAUUUCUACUAAGUGUAGAUCGAGUCG |
| | Cryptococcus 28S | | CGUUACUUGGGAG |
| | Cas12 Variant crRNA targeting | 961 | UAAUUUCUACUAAGUGUAGAUAUGGCCG |
| | Cryptococcus 28S | | GGGUUCGCCCACG |
| | Cas12 Variant crRNA targeting | 962 | UAAUUUCUACUAAGUGUAGAUCGGGCGU |
| | Cryptococcus 28S | | CCGAGUUGUAAUC |
| | Cas12 Variant crRNA targeting | 963 | UAAUUUCUACUAAGUGUAGAUACGCCCC |
| | Cryptococcus CTX1 | | GACGCGCCCAUUA |
| | Cas12 Variant crRNA targeting | 964 | UAAUUUCUACUAAGUGUAGAUGCCGGAC |
| | Cryptococcus CTX1 | | UGACGCACCCUAU |
| | Cas12 Variant crRNA targeting | 965 | UAAUUUCUACUAAGUGUAGAUCAGGUA |
| | Cryptococcus CTX1 | | UUGUGCGCGCCGAC |
| | Cas12 Variant crRNA targeting | 966 | UAAUUUCUACUAAGUGUAGAUUGUAGC |
| | Actinomyces 16S | | AUGCGUGAAGCCCA |
| | Cas12 Variant crRNA targeting | 967 | UAAUUUCUACUAAGUGUAGAUGGGACU |
| | Actinomyces 16S | | UAACCCAACAUCUC |
| | Cas12 Variant crRNA targeting | 968 | UAAUUUCUACUAAGUGUAGAUGGCUGCU |
| | Actinomyces 16S | | GGCACGUAGUUAG |
| | Cas12 Variant crRNA targeting | 969 | UAAUUUCUACUAAGUGUAGAUUGUAGC |
| | Streptococcus 16S | | ACGUGUGUAGCCCA |
| | Cas12 Variant crRNA targeting | 970 | UAAUUUCUACUAAGUGUAGAUCUAAUAC |
| | Streptococcus 16S | | AACGCAGGUCCAU |
| | Cas12 Variant crRNA targeting | 967 | UAAUUUCUACUAAGUGUAGAUGGGACU |
| | Streptococcus 16S | | UAACCCAACAUCUC |
| | Cas12 Variant crRNA targeting | 971 | UAAUUUCUACUAAGUGUAGAUACCAACG |
| | Streptococcus tuf | | CAUGAGGGAGUCA |
| | Cas12 Variant crRNA targeting | 972 | UAAUUUCUACUAAGUGUAGAUCUACAUG |
| | Streptococcus tuf | | ACUUGAACACGCG |
| | Cas12 Variant crRNA targeting | 973 | UAAUUUCUACUAAGUGUAGAUUUCGUA |
| | Streptococcus tuf | | GUCAGGUACUCUAU |
| | Cas12 Variant crRNA targeting | 974 | UAAUUUCUACUAAGUGUAGAUGGCGGU |
| | Streptococcus sodA | | AACACGGGUUCGAA |
| | Cas12 Variant crRNA targeting | 975 | UAAUUUCUACUAAGUGUAGAUGGGGUU |
| | Streptococcus sodA | | AAGACACCGCCUUU |
| | Cas12 Variant crRNA targeting | 976 | UAAUUUCUACUAAGUGUAGAUUGGUCA |
| | Streptococcus sodA | | AGGGGUUAAGACAC |
| | Cas12 Variant crRNA targeting | 975 | UAAUUUCUACUAAGUGUAGAUGGGGUU |
| | Streptococcus rpoB | | AAGACACCGCCUUU |
| | Cas12 Variant crRNA targeting | 974 | UAAUUUCUACUAAGUGUAGAUGGCGGU |
| | Streptococcus rpoB | | AACACGGGUUCGAA |
| | Cas12 Variant crRNA targeting | 976 | UAAUUUCUACUAAGUGUAGAUUGGUCA |
| | Streptococcus rpoB | | AGGGGUUAAGACAC |
| | CasM.26 crRNA targeting | 977 | GCCACCCCAAAAAUGAAGGGGACUAAAA |
| | Aspergillus 18S | | CAGGCGCAAUGUGCGUUCAAAG |
| | CasM.26 crRNA targeting | 978 | GCCACCCCAAAAAUGAAGGGGACUAAAA |
| | Aspergillus 18S | | CACGCUGCGUUCUUCAUCGAUG |
| | CasM.26 crRNA targeting | 979 | GCCACCCCAAAAAUGAAGGGGACUAAAA |
| | Aspergillus 18S | | CAGGCAGCAAUGACGCUCGGAC |
| | CasM.26 crRNA targeting | 980 | GCCACCCCAAAAAUGAAGGGGACUAAAA |
| | Aspergillus 28S | | CAGAAGCGCGUUCCUCGGUCCA |
| | CasM.26 crRNA targeting | 981 | GCCACCCCAAAAAUGAAGGGGACUAAAA |
| | Aspergillus 28S | | CAAAGCGCGUUCCUCGGUCCAG |
| | CasM.26 crRNA targeting | 982 | GCCACCCCAAAAAUGAAGGGGACUAAAA |
| | Aspergillus 28S | | CAAGCGCGUUCCUCGGUCCAGG |
| | CasM.26 crRNA targeting | 983 | GCCACCCCAAAAAUGAAGGGGACUAAAA |
| | Aspergillus ITS | | CAAUCCAUUGUUGAAAGUUUUA |
| | CasM.26 crRNA targeting | 984 | GCCACCCCAAAAAUGAAGGGGACUAAAA |
| | Aspergillus ITS | | CAGAGAUCCAUUGUUGAAAGUU |
| | CasM.26 crRNA targeting | 985 | GCCACCCCAAAAAUGAAGGGGACUAAAA |
| | Aspergillus ITS | | CAACCAAGAGAUCCAUUGUUGA |
| | CasM.26 crRNA targeting | 986 | GCCACCCCAAAAAUGAAGGGGACUAAAA |
| | Candida ITS | | CACUUCAUCGAUGCGAGAACCA |
| | CasM.26 crRNA targeting | 987 | GCCACCCCAAAAAUGAAGGGGACUAAAA |
| | Candida ITS | | CAGACGCUCAAACAGGCAUGCC |
| | CasM.26 crRNA targeting | 988 | GCCACCCCAAAAAUGAAGGGGACUAAAA |
| | Candida ITS | | CACAUCGAUGCGAGAACCAAGA |
| | CasM.26 crRNA targeting | 989 | GCCACCCCAAAAAUGAAGGGGACUAAAA |
| | Candida 28S | | CAUGUUCGCUAUCGGUCUCUCG |
| | CasM.26 crRNA targeting | 990 | GCCACCCCAAAAAUGAAGGGGACUAAAA |
| | Candida 28S | | CACCCUUUCAACAAUUUCACGU |
| | CasM.26 crRNA targeting | 991 | GCCACCCCAAAAAUGAAGGGGACUAAAA |
| | Candida 28S | | CACAUUCCCAAACAACUCGACU |
| | CasM.26 crRNA targeting | 992 | GCCACCCCAAAAAUGAAGGGGACUAAAA |
| | Candida Mitochondrion | | CAUGAGUGAUUAGUGCGAAAUA |
| | CasM.26 crRNA targeting | 993 | GCCACCCCAAAAAUGAAGGGGACUAAAA |
| | Candida Mitochondrion | | CAGACGACAACAAUGUAACGCC |
| | CasM.26 crRNA targeting | 994 | GCCACCCCAAAAAUGAAGGGGACUAAAA |
| | Candida Mitochondrion | | CAAUGGGUACCGAAUCCAUGUC |
| | CasM.26 crRNA targeting | 995 | GCCACCCCAAAAAUGAAGGGGACUAAAA |
| | Candida albicans ERG11 | | CACACGUCUCCAAUAAUGAGGU |
| | CasM.26 crRNA targeting | 996 | GCCACCCCAAAAAUGAAGGGGACUAAAA |
| | Candida albicans ERG11 | | CAGCAUCACGUCUCCAAUAAUG |
| | CasM.26 crRNA targeting | 997 | GCCACCCCAAAAAUGAAGGGGACUAAAA |
| | Candida albicans ERG11 | | CAAAUUGAGCAAAUGAACGGUC |
| | CasM.26 crRNA targeting | 998 | GCCACCCCAAAAAUGAAGGGGACUAAAA |
| | Candida lusitaniae ERG9 | | CACGCGUUCAAAACCAAGUCGU |
| | CasM.26 crRNA targeting | 999 | GCCACCCCAAAAAUGAAGGGGACUAAAA |
| | Candida lusitaniae ERG9 | | CAUCGUUAAUGCACUCGACACC |
| | CasM.26 crRNA targeting | 1000 | GCCACCCCAAAAAUGAAGGGGACUAAAA |
| | Candida lusitaniae ERG9 | | CAGACAUGGUUUAACGCGUUCA |
| | CasM.26 crRNA targeting | 1001 | GCCACCCCAAAAAUGAAGGGGACUAAAA |
| | Clostridium difficile 16S | | CAGAUAAUUCGGCUUCGGGCGU |
| | CasM.26 crRNA targeting | 1002 | GCCACCCCAAAAAUGAAGGGGACUAAAA |
| | Clostridium difficile 16S | | CAUCGGGCGUUUCCAACUCCCG |
| | CasM.26 crRNA targeting | 1003 | GCCACCCCAAAAAUGAAGGGGACUAAAA |
| | Clostridium difficile 16S | | CAUAGAUAAUUCGGCUUCGGGC |
| | CasM.26 crRNA targeting | 1004 | GCCACCCCAAAAAUGAAGGGGACUAAAA |
| | Clostridium perfringens 16S | | CAAUCGCUGACCCUACCUUCGG |
| | CasM.26 crRNA targeting | 1005 | GCCACCCCAAAAAUGAAGGGGACUAAAA |
| | Clostridium perfringens 16S | | CACGGACUUCGGGUAUUGCCAA |
| | CasM.26 crRNA targeting | 1006 | GCCACCCCAAAAAUGAAGGGGACUAAAA |
| | Clostridium perfringens 16S | | CACUUGCGGUUAGCUCACGGAC |
| | CasM.26 crRNA targeting | 1007 | GCCACCCCAAAAAUGAAGGGGACUAAAA |
| | Clostridium tetani 16S | | CAUUAACCCCACCUUCGGCCGC |
| | CasM.26 crRNA targeting | 1008 | GCCACCCCAAAAAUGAAGGGGACUAAAA |
| | Clostridium tetani 16S | | CAUAGCAACUCCGGCUUCAUGC |
| | CasM.26 crRNA targeting | 1009 | GCCACCCCAAAAAUGAAGGGGACUAAAA |
| | Clostridium tetani 16S | | CACGGACUUCGGGUGUUGCCAA |
| | CasM.26 crRNA targeting | 1010 | GCCACCCCAAAAAUGAAGGGGACUAAAA |
| | Campylobacteraceae 16S | | CACUCGCGGUAUUGCGUCUCAU |
| | CasM.26 crRNA targeting | 1011 | GCCACCCCAAAAAUGAAGGGGACUAAAA |
| | Campylobacteraceae 16S | | CACAAUCCGAACUGGGACAUAU |
| | CasM.26 crRNA targeting | 1012 | GCCACCCCAAAAAUGAAGGGGACUAAAA |
| | Campylobacteraceae 16S | | CAAUAGAUUUGCUCCACCUCGC |
| | CasM.26 crRNA targeting | 1013 | GCCACCCCAAAAAUGAAGGGGACUAAAA |
| | Campylobacteraceae 23S | | CACCAAACUUAGCUACGCUGCG |
| | CasM.26 crRNA targeting | 1014 | GCCACCCCAAAAAUGAAGGGGACUAAAA |
| | Campylobacteraceae 23S | | CAGUGGUUUGUUCAACCCGGUC |
| | CasM.26 crRNA targeting | 1015 | GCCACCCCAAAAAUGAAGGGGACUAAAA |
| | Campylobacteraceae 23S | | CAUUAGCUACGCUGCGAUGCUC |
| | CasM.26 crRNA targeting | 1016 | GCCACCCCAAAAAUGAAGGGGACUAAAA |
| | Campylobacteraceae flaG | | CAGAUAAGAAGAAUCCGCACAA |
| | CasM.26 crRNA targeting | 1017 | GCCACCCCAAAAAUGAAGGGGACUAAAA |
| | Campylobacteraceae flaG | | CAAUAGGAUAAGAAGAAUCCGC |
| | CasM.26 crRNA targeting | 1018 | GCCACCCCAAAAAUGAAGGGGACUAAAA |
| | Campylobacteraceae flaG | | CAUAGGAUAAGAAGAAUCCGCA |
| | CasM.26 crRNA targeting | 1019 | GCCACCCCAAAAAUGAAGGGGACUAAAA |
| | Corynebacterium 16S | | CACGGCUUUCAGCGAUUCGCUC |
| | CasM.26 crRNA targeting | 1020 | GCCACCCCAAAAAUGAAGGGGACUAAAA |
| | Corynebacterium 16S | | CAGCGUUGUACCGACCAUUGUA |
| | CasM.26 crRNA targeting | 1021 | GCCACCCCAAAAAUGAAGGGGACUAAAA |
| | Corynebacterium 16S | | CAGGCCGGCUUUCAGCGAUUCG |
| | CasM.26 crRNA targeting | 1022 | GCCACCCCAAAAAUGAAGGGGACUAAAA |
| | Corynebacterium rpoB | | CAGGACGCUCGGUGGACUUAUC |
| | CasM.26 crRNA targeting | 1023 | GCCACCCCAAAAAUGAAGGGGACUAAAA |
| | Corynebacterium rpoB | | CAUGAGAAGCGUGAACGUCUCG |
| | CasM.26 crRNA targeting | 1024 | GCCACCCCAAAAAUGAAGGGGACUAAAA |
| | Corynebacterium rpoB | | CAUGGCCGAUGUUCAUACGACG |
| | CasM.26 crRNA targeting | 1025 | GCCACCCCAAAAAUGAAGGGGACUAAAA |
| | Corynebacterium tox | | CAUACAUCGCAUCUUGGCCACG |
| | CasM.26 crRNA targeting | 1026 | GCCACCCCAAAAAUGAAGGGGACUAAAA |
| | Corynebacterium tox | | CAGAGCACUACACGCGAAGCAC |
| | CasM.26 crRNA targeting | 1027 | GCCACCCCAAAAAUGAAGGGGACUAAAA |
| | Corynebacterium tox | | CACGAAGCACCAUCACCGAACC |
| | CasM.26 crRNA targeting | 1028 | GCCACCCCAAAAAUGAAGGGGACUAAAA |
| | Corynebacterium diphtheriae | | CAGUGCAUUGAUUAGUUCGCCG |
| | chrA | | |
| | CasM.26 crRNA targeting | 1029 | GCCACCCCAAAAAUGAAGGGGACUAAAA |
| | Corynebacterium diphtheriae | | CAAUCGCUUCCAAACCGGCCGU |
| | chrA | | |
| | CasM.26 crRNA targeting | 1030 | GCCACCCCAAAAAUGAAGGGGACUAAAA |
| | Corynebacterium diphtheriae | | CAACGGCCGGUUUGGAAGCGAU |
| | chrA | | |
| | CasM.26 crRNA targeting | 1031 | GCCACCCCAAAAAUGAAGGGGACUAAAA |
| | Corynebacterium | | CAACUUUAUGUGAGUCGCCGCG |
| | pseudotuberculosis chrA | | |
| | CasM.26 crRNA targeting | 1032 | GCCACCCCAAAAAUGAAGGGGACUAAAA |
| | Corynebacterium | | CAAUUAGUGAGUGUAAGGCGAG |
| | pseudotuberculosis chrA | | |
| | CasM.26 crRNA targeting | 1033 | GCCACCCCAAAAAUGAAGGGGACUAAAA |
| | Corynebacterium | | CACGACCGACAGCGCCCCUCAC |
| | pseudotuberculosis chrA | | |
| | CasM.26 crRNA targeting | 1034 | GCCACCCCAAAAAUGAAGGGGACUAAAA |
| | Corynebacterium | | CACGAGCUUCGCUGGACUUACG |
| | pseudotuberculosis pld | | |
| | CasM.26 crRNA targeting | 1035 | GCCACCCCAAAAAUGAAGGGGACUAAAA |
| | Corynebacterium | | CAAAGAUCAUUAACUCGCGCGU |
| | pseudotuberculosis pld | | |
| | CasM.26 crRNA targeting | 1036 | GCCACCCCAAAAAUGAAGGGGACUAAAA |
| | Corynebacterium | | CAAUUAACUCGCGCGUCCUGAC |
| | pseudotuberculosis pld | | |
| | CasM.26 crRNA targeting | 1037 | GCCACCCCAAAAAUGAAGGGGACUAAAA |
| | Corynebacterium | | CAUCGCAACUACGCGAAGCAAC |
| | striatum chrA | | |
| | CasM.26 crRNA targeting | 1038 | GCCACCCCAAAAAUGAAGGGGACUAAAA |
| | Corynebacterium | | CACCACCGCAUCGCAACUACGC |
| | striatum chrA | | |
| | CasM.26 crRNA targeting | 1039 | GCCACCCCAAAAAUGAAGGGGACUAAAA |
| | Corynebacterium | | CAGUUGUCCGGAAUGUGACCCG |
| | striatum chrA | | |
| | CasM.26 crRNA targeting | 1040 | GCCACCCCAAAAAUGAAGGGGACUAAAA |
| | Corynebacterium ulcerans pld | | CAACCGUUCGCACCACGCUUAU |
| | CasM.26 crRNA targeting | 1041 | GCCACCCCAAAAAUGAAGGGGACUAAAA |
| | Corynebacterium ulcerans pld | | CACUUAUCGACGACCAACUCAU |
| | CasM.26 crRNA targeting | 1042 | GCCACCCCAAAAAUGAAGGGGACUAAAA |
| | Corynebacterium ulcerans pld | | CAUUGUCGCGGUACUCCACGAU |
| | CasM.26 crRNA targeting | 1043 | GCCACCCCAAAAAUGAAGGGGACUAAAA |
| | Escherichia coli O157:H1 | | CAACGUGAAGCGCGGUUUGCGA |
| | pO157 | | |
| | CasM.26 crRNA targeting | 1044 | GCCACCCCAAAAAUGAAGGGGACUAAAA |
| | Escherichia coli O157:H1 | | CACGAAACGGCGUUGCCCCUUA |
| | pO157 | | |
| | CasM.26 crRNA targeting | 1045 | GCCACCCCAAAAAUGAAGGGGACUAAAA |
| | Escherichia coli O157:H1 | | CAAACGACGUGAAGCGCGGUUU |
| | pO157 | | |
| | CasM.26 crRNA targeting | 1046 | GCCACCCCAAAAAUGAAGGGGACUAAAA |
| | Escherichia coli O0157:H1 | | CAAUCGCGUUGUCAUACGAGUC |
| | stx2A | | |
| | CasM.26 crRNA targeting | 1047 | GCCACCCCAAAAAUGAAGGGGACUAAAA |
| | Escherichia coli O157:H1 | | CACCAGCAAUCGUCGAUUGUUA |
| | stx2A | | |
| | CasM.26 crRNA targeting | 1048 | GCCACCCCAAAAAUGAAGGGGACUAAAA |
| | Escherichia coli O157:H1 | | CAGCUCUUACGGUGUAUUACCU |
| | stx2A | | |
| | CasM.26 crRNA targeting | 1049 | GCCACCCCAAAAAUGAAGGGGACUAAAA |
| | Shiga toxin stx | | CACGGGUUAACCCAAUCGGCUU |
| | CasM.26 crRNA targeting | 1050 | GCCACCCCAAAAAUGAAGGGGACUAAAA |
| | Shiga toxin stx | | CAAUAGCCCGUCAUUUCUGCGC |
| | CasM.26 crRNA targeting | 1051 | GCCACCCCAAAAAUGAAGGGGACUAAAA |
| | Shiga toxin stx | | CAGUUGGUUACGUAUCGUUUAA |
| | CasM.26 crRNA targeting | 1052 | GCCACCCCAAAAAUGAAGGGGACUAAAA |
| | Listeria monocytogenes 16S | | CAGCUUCGCGACCCUUUGUACU |
| | CasM.26 crRNA targeting | 1053 | GCCACCCCAAAAAUGAAGGGGACUAAAA |
| | Listeria monocytogenes 16S | | CACACCUCGCGGCUUCGCGACC |
| | CasM.26 crRNA targeting | 1054 | GCCACCCCAAAAAUGAAGGGGACUAAAA |
| | Listeria monocytogenes 16S | | CAAUAAAGGUGACCCUACCGAC |
| | CasM.26 crRNA targeting | 1055 | GCCACCCCAAAAAUGAAGGGGACUAAAA |
| | Listeria monocytogenes 23S | | CACAGCGAUGCUCCUGGCGGAA |
| | CasM.26 crRNA targeting | 1056 | GCCACCCCAAAAAUGAAGGGGACUAAAA |
| | Listeria monocytogenes 23S | | CAAGGGAUCUUACUUUCCGAAG |
| | CasM.26 crRNA targeting | 1057 | GCCACCCCAAAAAUGAAGGGGACUAAAA |
| | Listeria monocytogenes 23S | | CAUAGCUACCCAGCGAUGCUCC |
| | CasM.26 crRNA targeting | 1058 | GCCACCCCAAAAAUGAAGGGGACUAAAA |
| | Listeria monocytogenes prfA | | CAUUCGUAAACGUACGUAACGG |
| | CasM.26 crRNA targeting | 1059 | GCCACCCCAAAAAUGAAGGGGACUAAAA |
| | Listeria monocytogenes prfA | | CACGUACGUAACGGUUUGUUUA |
| | CasM.26 crRNA targeting | 1060 | GCCACCCCAAAAAUGAAGGGGACUAAAA |
| | Listeria monocytogenes prfA | | CAAUCUACCGGAUCUACCGGAU |
| | CasM.26 crRNA targeting | 1061 | GCCACCCCAAAAAUGAAGGGGACUAAAA |
| | Mycobacterium bovis 16S | | CACGACAAACCACUACGAGCUC |
| | CasM.26 crRNA targeting | 1062 | GCCACCCCAAAAAUGAAGGGGACUAAAA |
| | Mycobacterium bovis 16S | | CAACAACGCGACAAACCACUAC |
| | CasM.26 crRNA targeting | 1063 | GCCACCCCAAAAAUGAAGGGGACUAAAA |
| | Mycobacterium bovis 16S | | CAACCACUACGAGCUCUUUACG |
| | CasM.26 crRNA targeting | 1064 | GCCACCCCAAAAAUGAAGGGGACUAAAA |
| | Neisseria meningitidis 16S | | CAGUAAGCGGGCUCCUUGCGGU |
| | CasM.26 crRNA targeting | 1065 | GCCACCCCAAAAAUGAAGGGGACUAAAA |
| | Neisseria meningitidis 16S | | CACCGUGGUAAGCGGGCUCCUU |
| | CasM.26 crRNA targeting | 1066 | GCCACCCCAAAAAUGAAGGGGACUAAAA |
| | Neisseria meningitidis 16S | | CACGGGCUCCUUGCGGUUACCC |
| | CasM.26 crRNA targeting | 1067 | GCCACCCCAAAAAUGAAGGGGACUAAAA |
| | Neisseria meningitidis 23S | | CACGAACGACUCUUUAGUGCGG |
| | CasM.26 crRNA targeting | 1068 | GCCACCCCAAAAAUGAAGGGGACUAAAA |
| | Neisseria meningitidis 23S | | CAUAGUGCGGUUAAACCGCAAG |
| | CasM.26 crRNA targeting | 1069 | GCCACCCCAAAAAUGAAGGGGACUAAAA |
| | Neisseria meningitidis 23S | | CACGGCUAUGCAACUGGCGUUA |
| | CasM.26 crRNA targeting | 1070 | GCCACCCCAAAAAUGAAGGGGACUAAAA |
| | Salmonella enterica Paratyphi | | CAUCGCCUGCGCCAGAACGGAG |
| | filC | | |
| | CasM.26 crRNA targeting | 1071 | GCCACCCCAAAAAUGAAGGGGACUAAAA |
| | Salmonella enterica Paratyphi | | CAGUUUUGCGGAACCUGGUUCG |
| | filC | | |
| | CasM.26 crRNA targeting | 1072 | GCCACCCCAAAAAUGAAGGGGACUAAAA |
| | Salmonella enterica Paratyphi | | CAACCGCACCCAAGUCAGAACG |
| | filC | | |
| | CasM.26 crRNA targeting | 1073 | GCCACCCCAAAAAUGAAGGGGACUAAAA |
| | Salmonella enterica Typhi | | CACGGGAUCGAUAAUUGCUCAC |
| | segment 2/20 | | |
| | CasM.26 crRNA targeting | 1074 | GCCACCCCAAAAAUGAAGGGGACUAAAA |
| | Salmonella enterica Typhi | | CACCGGAUACGGGAUCGAUAAU |
| | segment 2/20 | | |
| | CasM.26 crRNA targeting | 1075 | GCCACCCCAAAAAUGAAGGGGACUAAAA |
| | Salmonella enterica Typhi | | CAAGUCCUGUAUCUUCGCCGUA |
| | segment 2/20 | | |
| | CasM.26 crRNA targeting | 1076 | GCCACCCCAAAAAUGAAGGGGACUAAAA |
| | Salmonella enterica Typhi 16S | | CAGCGCCCCCCCGAAGGUUAAG |
| | CasM.26 crRNA targeting | 1077 | GCCACCCCAAAAAUGAAGGGGACUAAAA |
| | Salmonella enterica Typhi 16S | | CACCCCGAAGGUUAAGCUACCU |
| | CasM.26 crRNA targeting | 1078 | GCCACCCCAAAAAUGAAGGGGACUAAAA |
| | Salmonella enterica Typhi 16S | | CACACAAAGUGGUAAGCGCCCC |
| | CasM.26 crRNA targeting | 1079 | GCCACCCCAAAAAUGAAGGGGACUAAAA |
| | Salmonella enterica Enteritides | | CAUGAUGAGCGACAUUACCGUA |
| | difference region 1 | | |
| | CasM.26 crRNA targeting | 1080 | GCCACCCCAAAAAUGAAGGGGACUAAAA |
| | Salmonella enterica Enteritides | | CAGAACAAUGUUGAUGAGCGAC |
| | difference region 1 | | |
| | CasM.26 crRNA targeting | 1081 | GCCACCCCAAAAAUGAAGGGGACUAAAA |
| | Salmonella enterica Enteritides | | CACGUUCUGUGCUGGACAUACG |
| | difference region 1 | | |
| | CasM.26 crRNA targeting | 1082 | GCCACCCCAAAAAUGAAGGGGACUAAAA |
| | Salmonella enterica Enteritides | | CAACGUAUCUGAUCCCAGAAUG |
| | caiC | | |
| | CasM.26 crRNA targeting | 1083 | GCCACCCCAAAAAUGAAGGGGACUAAAA |
| | Salmonella enterica Enteritides | | CAUCGCCUGAUAUUUACGUAUC |
| | caiC | | |
| | CasM.26 crRNA targeting | 1084 | GCCACCCCAAAAAUGAAGGGGACUAAAA |
| | Salmonella enterica Enteritides | | CAUCUGAUCCCAGAAUGCCCGG |
| | caiC | | |
| | CasM.26 crRNA targeting | 1085 | GCCACCCCAAAAAUGAAGGGGACUAAAA |
| | Yersinia enterocolitica 16S | | CACGACAGACUUUAUGUGGUCC |
| | CasM.26 crRNA targeting | 1086 | GCCACCCCAAAAAUGAAGGGGACUAAAA |
| | Yersinia enterocolitica 16S | | CAGACUACGACAGACUUUAUGU |
| | CasM.26 crRNA targeting | 1087 | GCCACCCCAAAAAUGAAGGGGACUAAAA |
| | Yersinia enterocolitica 16S | | CAAGACUUUAUGUGGUCCGCUU |
| | CasM.26 crRNA targeting | 1088 | GCCACCCCAAAAAUGAAGGGGACUAAAA |
| | Yersinia enterocolitica 23S | | CAUCAGGGGGCUUUAAGCCCCA |
| | CasM.26 crRNA targeting | 1089 | GCCACCCCAAAAAUGAAGGGGACUAAAA |
| | Yersinia enterocolitica 23S | | CAGGGCUUUAAGCCCCAGGGAA |
| | CasM.26 crRNA targeting | 1090 | GCCACCCCAAAAAUGAAGGGGACUAAAA |
| | Yersinia enterocolitica 23S | | CAUUAACGUUCCUUCAGGGGGC |
| | CasM.26 crRNA targeting | 1091 | GCCACCCCAAAAAUGAAGGGGACUAAAA |
| | Yersinia pseudotuberculosis | | CACGAGGCAAGUUUCCUGCUUA |
| | 23S | | |
| | CasM.26 crRNA targeting | 1092 | GCCACCCCAAAAAUGAAGGGGACUAAAA |
| | Yersinia pseudotuberculosis | | CACAUCUCGAGGCAAGUUUCCU |
| | 23S | | |
| | CasM.26 crRNA targeting | 1093 | GCCACCCCAAAAAUGAAGGGGACUAAAA |
| | Yersinia pseudotuberculosis | | CACUGCUUAGAUGCUUUCAGCG |
| | 23S | | |
| | CasM.26 crRNA targeting | 1094 | GCCACCCCAAAAAUGAAGGGGACUAAAA |
| | Vibrio cholerae 16S | | CAACGACGUACUUUGUGAGAUU |
| | CasM.26 crRNA targeting | 1095 | GCCACCCCAAAAAUGAAGGGGACUAAAA |
| | Vibrio cholerae 16S | | CACGCAACAUUCUGAUUUGCGA |
| | CasM.26 crRNA targeting | 1096 | GCCACCCCAAAAAUGAAGGGGACUAAAA |
| | Vibrio cholerae 16S | | CAACCACAAAGUGGCAAGCGUC |
| | CasM.26 crRNA targeting | 1097 | GCCACCCCAAAAAUGAAGGGGACUAAAA |
| | Vibrio cholerae hlyA | | CAACGCUACGCGUUGACAGUCC |
| | CasM.26 crRNA targeting | 1098 | GCCACCCCAAAAAUGAAGGGGACUAAAA |
| | Vibrio cholerae hlyA | | CAGCCGCGCGUUGACAGUCCCU |
| | CasM.26 crRNA targeting | 1099 | GCCACCCCAAAAAUGAAGGGGACUAAAA |
| | Vibrio cholerae hly A | | CACACAUAACGCCCGCCUAAGG |
| | CasM.26 crRNA targeting | 1100 | GCCACCCCAAAAAUGAAGGGGACUAAAA |
| | Vibrio parahaemolyticus 23S | | CAGUAAGCGUCCCCCCGAAGGU |
| | CasM.26 crRNA targeting | 1101 | GCCACCCCAAAAAUGAAGGGGACUAAAA |
| | Vibrio parahaemolyticus 23S | | CACCCCGAAGGUUAAACUACCU |
| | CasM.26 crRNA targeting | 1102 | GCCACCCCAAAAAUGAAGGGGACUAAAA |
| | Vibrio parahaemolyticus 23S | | CACACAAAGUGGUAAGCGUCCC |
| | CasM.26 crRNA targeting | 1103 | GCCACCCCAAAAAUGAAGGGGACUAAAA |
| | Vibrio vulnificus 16S | | CAACCACAAAGUGGUGAGCGUC |
| | CasM.26 crRNA targeting | 1104 | GCCACCCCAAAAAUGAAGGGGACUAAAA |
| | Vibrio vulnificus 16S | | CAAGCGCCAGGUUCCCCUAGCG |
| | CasM.26 crRNA targeting | 1105 | GCCACCCCAAAAAUGAAGGGGACUAAAA |
| | Vibrio vulnificus 16S | | CACCAGGUUCCCCUAGCGCUAC |
| | CasM.26 crRNA targeting | 1106 | GCCACCCCAAAAAUGAAGGGGACUAAAA |
| | Vibrio vulnificus 23S | | CACGAACAACCCUUCAGGAUAC |
| | CasM.26 crRNA targeting | 1107 | GCCACCCCAAAAAUGAAGGGGACUAAAA |
| | Vibrio vulnificus 23S | | CACAGGAUACUUAAAGUAUCAG |
| | CasM.26 crRNA targeting | 1108 | GCCACCCCAAAAAUGAAGGGGACUAAAA |
| | Vibrio vulnificus 23S | | CAACCCUUCAGGAUACUUAAAG |
| | CasM.26 crRNA targeting | 1109 | GCCACCCCAAAAAUGAAGGGGACUAAAA |
| | Cronobacter flk | | CAGCGGGUGCGCUUUGCUUACC |
| | CasM.26 crRNA targeting | 1110 | GCCACCCCAAAAAUGAAGGGGACUAAAA |
| | Cronobacter flk | | CAGGUGGGUAAGCGAAGCGCAC |
| | CasM.26 crRNA targeting | 1111 | GCCACCCCAAAAAUGAAGGGGACUAAAA |
| | Cronobacter flk | | CAGUGCGCUUCGCUUACCCACC |
| | CasM.26 crRNA targeting | 175 | GCCACCCCAAAAAUGAAGGGGACUAAAA |
| | Alphacoronavirus complete | | CAUCUAGUCGAGCUGCACUAGA |
| | genome | | |
| | CasM.26 crRNA targeting | 1112 | GCCACCCCAAAAAUGAAGGGGACUAAAA |
| | Alphacoronavirus complete | | CAUUACCACUACCAGGAGGACC |
| | genome | | |
| | CasM.26 crRNA targeting | 1113 | GCCACCCCAAAAAUGAAGGGGACUAAAA |
| | Alphacoronavirus complete | | CACCCCGCACUCGUUUAAAUAA |
| | genome | | |
| | CasM.26 crRNA targeting | 1114 | GCCACCCCAAAAAUGAAGGGGACUAAAA |
| | Betacoronavirus complete | | CAGGAUAAUCCCAACCCAUAAG |
| | genome | | |
| | CasM.26 crRNA targeting | 1115 | GCCACCCCAAAAAUGAAGGGGACUAAAA |
| | Betacoronavirus complete | | CAUUAGGAUAAUCCCAACCCAU |
| | genome | | |
| | CasM.26 crRNA targeting | 1116 | GCCACCCCAAAAAUGAAGGGGACUAAAA |
| | Betacoronavirus complete | | CACUAUUCUUAGCACUAAUAGC |
| | genome | | |
| | CasM.26 crRNA targeting | 1117 | GCCACCCCAAAAAUGAAGGGGACUAAAA |
| | Deltacoronavirus complete | | CAUGUACCCUCGAUCGUACUCC |
| | genome | | |
| | CasM.26 crRNA targeting | 1118 | GCCACCCCAAAAAUGAAGGGGACUAAAA |
| | Deltacoronavirus complete | | CAUCGUACUCCGCGUGGCCUCG |
| | genome | | |
| | CasM.26 crRNA targeting | 1119 | GCCACCCCAAAAAUGAAGGGGACUAAAA |
| | Deltacoronavirus complete | | CACCUCGAUCGUACUCCGCGUG |
| | genome | | |
| | CasM.26 crRNA targeting | 1120 | GCCACCCCAAAAAUGAAGGGGACUAAAA |
| | Cytomegalovirus complete | | CAUCCGCGUGUGUAAACGGCGU |
| | genome | | |
| | CasM.26 crRNA targeting | 1121 | GCCACCCCAAAAAUGAAGGGGACUAAAA |
| | Cytomegalovirus complete | | CACGUGUGUCCUCGACGCGGGU |
| | genome | | |
| | CasM.26 crRNA targeting | 1122 | GCCACCCCAAAAAUGAAGGGGACUAAAA |
| | Cytomegalovirus complete | | CAAUACCGGCGCAUGCGCACGA |
| | genome | | |
| | CasM.26 crRNA targeting | 1123 | GCCACCCCAAAAAUGAAGGGGACUAAAA |
| | Goatpox virus P32 | | CAGUGCUCCCAUUAUACUAAUA |
| | CasM.26 crRNA targeting | 1124 | GCCACCCCAAAAAUGAAGGGGACUAAAA |
| | Goatpox virus P32 | | CAUACCUGCUAAAAGCCAUAGU |
| | CasM.26 crRNA targeting | 1125 | GCCACCCCAAAAAUGAAGGGGACUAAAA |
| | Goatpox virus P32 | | CAUAAGUGCUCCCAUUAUACUA |
| | CasM.26 crRNA targeting | 1126 | GCCACCCCAAAAAUGAAGGGGACUAAAA |
| | Sheeppox virus P32 | | CACUUGUCUAUUUAGUUUUAGU |
| | CasM.26 crRNA targeting | 1127 | GCCACCCCAAAAAUGAAGGGGACUAAAA |
| | Sheeppox virus P32 | | CAGUCUAUUUAGUUUUAGUUCU |
| | CasM.26 crRNA targeting | 1128 | GCCACCCCAAAAAUGAAGGGGACUAAAA |
| | Sheeppox virus P32 | | CAUAGUUUUAGUUCUUUCUCUA |
| | CasM.26 crRNA targeting | 1129 | GCCACCCCAAAAAUGAAGGGGACUAAAA |
| | Mycoplasma capricolum | | CAUCUUACAUCUUAUGAAUGGG |
| | capripneumoniae 23S | | |
| | CasM.26 crRNA targeting | 1130 | GCCACCCCAAAAAUGAAGGGGACUAAAA |
| | Mycoplasma capricolum | | CAGGAUCUUACAUCUUAUGAAU |
| | capripneumoniae 23S | | |
| | CasM.26 crRNA targeting | 1131 | GCCACCCCAAAAAUGAAGGGGACUAAAA |
| | Mycoplasma capricolum | | CAGCACCAGAGGUGCAUCCAUU |
| | capripneumoniae 23S | | |
| | CasM.26 crRNA targeting | 1132 | GCCACCCCAAAAAUGAAGGGGACUAAAA |
| | Mycoplasma capricolum | | CAACCGUGAUCUCUAGGUCUAA |
| | capripneumoniae L22 | | |
| | CasM.26 crRNA targeting | 1133 | GCCACCCCAAAAAUGAAGGGGACUAAAA |
| | Mycoplasma capricolum | | CAUCAUAUGCUCUACCGUGAUC |
| | capripneumoniae L22 | | |
| | CasM.26 crRNA targeting | 1134 | GCCACCCCAAAAAUGAAGGGGACUAAAA |
| | Mycoplasma capricolum | | CAGAUUGUAUCUGCAACUAAUC |
| | capripneumoniae L22 | | |
| | CasM.26 crRNA targeting | 1135 | GCCACCCCAAAAAUGAAGGGGACUAAAA |
| | Mycoplasma mycoides | | CAUGGGAAGCGCUCUCCAAAUG |
| | mycoides 23S | | |
| | CasM.26 crRNA targeting | 1136 | GCCACCCCAAAAAUGAAGGGGACUAAAA |
| | Mycoplasma mycoides | | CAGCUCUCCAAAUGGUUAAGCU |
| | mycoides 23S | | |
| | CasM.26 crRNA targeting | 1137 | GCCACCCCAAAAAUGAAGGGGACUAAAA |
| | Mycoplasma mycoides | | CAAAUGGUUAAGCUACCUACUU |
| | mycoides 23S | | |
| | CasM.26 crRNA targeting | 1138 | GCCACCCCAAAAAUGAAGGGGACUAAAA |
| | Mycoplasma mycoides | | CAUAGAUCAGCUUCUAAUUUAC |
| | mycoides lppB | | |
| | CasM.26 crRNA targeting | 1139 | GCCACCCCAAAAAUGAAGGGGACUAAAA |
| | Mycoplasma mycoides | | CAUCGUUUUUAGAUUUUAGAUC |
| | mycoides lppB | | |
| | CasM.26 crRNA targeting | 1140 | GCCACCCCAAAAAUGAAGGGGACUAAAA |
| | Mycoplasma mycoides | | CAAGUUUCAAAUUGAGAGAUUA |
| | mycoides lppB | | |
| | CasM.26 crRNA targeting | 1141 | GCCACCCCAAAAAUGAAGGGGACUAAAA |
| | Gammacoronavirus complete | | CAACCCCGUACCCGUUUAAAUA |
| | genome | | |
| | CasM.26 crRNA targeting | 169 | GCCACCCCAAAAAUGAAGGGGACUAAAA |
| | Gammacoronavirus complete | | CACCCCGUACCCGUUUAAAUAA |
| | genome | | |
| | CasM.26 crRNA targeting | 1142 | GCCACCCCAAAAAUGAAGGGGACUAAAA |
| | Gammacoronavirus complete | | CACCGUACCCGUUUAAAUAAUU |
| | genome | | |
| | CasM.26 crRNA targeting | 1143 | GCCACCCCAAAAAUGAAGGGGACUAAAA |
| | Newcastle disease virus whole | | CAGCUAAGUCAAGUCUCGCAAA |
| | genome | | |
| | CasM.26 crRNA targeting | 1144 | GCCACCCCAAAAAUGAAGGGGACUAAAA |
| | Newcastle disease virus whole | | CAAAUGGACGGACGGGCUGUCC |
| | genome | | |
| | CasM.26 crRNA targeting | 1145 | GCCACCCCAAAAAUGAAGGGGACUAAAA |
| | Newcastle disease virus whole | | CAUUGAUGAUUACUACCCCGCC |
| | genome | | |
| | CasM.26 crRNA targeting | 1146 | GCCACCCCAAAAAUGAAGGGGACUAAAA |
| | Peste des petits ruminants | | CAUUAGUGAACUCGUCCGCACC |
| | virus (PPRV) whole genome | | |
| | CasM.26 crRNA targeting | 1147 | GCCACCCCAAAAAUGAAGGGGACUAAAA |
| | Peste des petits ruminants | | CACGUAUCGAGGGUGAACAACC |
| | virus (PPRV) whole genome | | |
| | CasM.26 crRNA targeting | 1148 | GCCACCCCAAAAAUGAAGGGGACUAAAA |
| | Peste des petits ruminants | | CAUCACUUCUUUCGUAUCGAGG |
| | virus (PPRV) whole genome | | |
| | CasM.26 crRNA targeting | 1149 | GCCACCCCAAAAAUGAAGGGGACUAAAA |
| | Porcine Reproductive and | | CACGACCGCCAACGAUAGAGUC |
| | Respriatory Syndrome (PRRS) | | |
| | virus - North American whole | | |
| | genome | | |
| | CasM.26 crRNA targeting | 1150 | GCCACCCCAAAAAUGAAGGGGACUAAAA |
| | Porcine Reproductive and | | CAGGUAUAUCUGGUACACGCGU |
| | Respriatory Syndrome (PRRS) | | |
| | virus - North American whole | | |
| | genome | | |
| | CasM.26 crRNA targeting | 1151 | GCCACCCCAAAAAUGAAGGGGACUAAAA |
| | Porcine Reproductive and | | CAGGACGACCCCAUUGUUCCGC |
| | Respriatory Syndrome (PRRS) | | |
| | virus - North American whole | | |
| | genome | | |
| | CasM.26 crRNA targeting | 1152 | GCCACCCCAAAAAUGAAGGGGACUAAAA |
| | Porcine Reproductive and | | CAGAAGUCACGCGAAUCAGGCG |
| | Respriatory Syndrome (PRRS) | | |
| | virus - European whole genome | | |
| | CasM.26 crRNA targeting | 1153 | GCCACCCCAAAAAUGAAGGGGACUAAAA |
| | Porcine Reproductive and | | CAGCGCCUUGAUUGAAAGCCGU |
| | Respriatory Syndrome (PRRS) | | |
| | virus - European whole genome | | |
| | CasM.26 crRNA targeting | 1154 | GCCACCCCAAAAAUGAAGGGGACUAAAA |
| | Porcine Reproductive and | | CAUAUGAGCAACCGGCAGCAUA |
| | Respriatory Syndrome (PRRS) | | |
| | virus - European whole genome | | |
| | CasM.26 crRNA targeting | 1155 | GCCACCCCAAAAAUGAAGGGGACUAAAA |
| | Porcine Reproductive and | | CAAUUGGUAUGUCGAGCUGUUG |
| | Respriatory Syndrome (PRRS) | | |
| | virus - Eastern European whole | | |
| | genome | | |
| | CasM.26 crRNA targeting | 1156 | GCCACCCCAAAAAUGAAGGGGACUAAAA |
| | Porcine Reproductive and | | CAUGUAUUGGUAUGUCGAGCUG |
| | Respriatory Syndrome (PRRS) | | |
| | virus - Eastern European whole | | |
| | genome | | |
| | CasM.26 crRNA targeting | 1157 | GCCACCCCAAAAAUGAAGGGGACUAAAA |
| | Porcine Reproductive and | | CAGGUAUGUCGAGCUGUUGCCG |
| | Respriatory Syndrome (PRRS) | | |
| | virus - Eastern European whole | | |
| | genome | | |
| | CasM.26 crRNA targeting | 1158 | GCCACCCCAAAAAUGAAGGGGACUAAAA |
| | Andes Orthohantavirus | | CAGAACCCUUACCCGUAGUUAG |
| | Segment S | | |
| | CasM.26 crRNA targeting | 1159 | GCCACCCCAAAAAUGAAGGGGACUAAAA |
| | Andes Orthohantavirus | | CAAGUAAUGAACCCUUACCCGU |
| | Segment S | | |
| | CasM.26 crRNA targeting | 1160 | GCCACCCCAAAAAUGAAGGGGACUAAAA |
| | Andes Orthohantavirus | | CAUAGGUUAGCAAUGCGAAAGC |
| | Segment S | | |
| | CasM.26 crRNA targeting | 1161 | GCCACCCCAAAAAUGAAGGGGACUAAAA |
| | Andes Orthohantavirus | | CAGGCACUCUGUUAAUCCGACA |
| | Segment M | | |
| | CasM.26 crRNA targeting | 1162 | GCCACCCCAAAAAUGAAGGGGACUAAAA |
| | Andes Orthohantavirus | | CAGGUCAAUCCAUUCAAGCUUA |
| | Segment M | | |
| | CasM.26 crRNA targeting | 1163 | GCCACCCCAAAAAUGAAGGGGACUAAAA |
| | Andes Orthohantavirus | | CAAUGACCUCCCUUACCAACUA |
| | Segment M | | |
| | CasM.26 crRNA targeting | 1164 | GCCACCCCAAAAAUGAAGGGGACUAAAA |
| | Andes Orthohantavirus | | CAGGGUCACGCGGGAACAACCU |
| | Segment L | | |
| | CasM.26 crRNA targeting | 1165 | GCCACCCCAAAAAUGAAGGGGACUAAAA |
| | Andes Orthohantavirus | | CAUACAGGGUCACGCGGGAACA |
| | Segment L | | |
| | CasM.26 crRNA targeting | 1166 | GCCACCCCAAAAAUGAAGGGGACUAAAA |
| | Andes Orthohantavirus | | CACAGACCACUGCUUGCUAGAC |
| | Segment L | | |
| | CasM.26 crRNA targeting | 1167 | GCCACCCCAAAAAUGAAGGGGACUAAAA |
| | Dobrava-Begrade | | CAUAAGGUGACGUCCUAAACCA |
| | orthohantavirus Segment S | | |
| | CasM.26 crRNA targeting | 1168 | GCCACCCCAAAAAUGAAGGGGACUAAAA |
| | Dobrava-Begrade | | CAGUGACGUCCUAAACCACCCU |
| | orthohantavirus Segment S | | |
| | CasM.26 crRNA targeting | 1169 | GCCACCCCAAAAAUGAAGGGGACUAAAA |
| | Dobrava-Begrade | | CAAGUCACUUAAGGUGACGUCC |
| | orthohantavirus Segment S | | |
| | CasM.26 crRNA targeting | 1170 | GCCACCCCAAAAAUGAAGGGGACUAAAA |
| | Dobrava-Begrade | | CACGUUUAUGCUUACGAAUGGG |
| | orthohantavirus Segment M | | |
| | CasM.26 crRNA targeting | 1171 | GCCACCCCAAAAAUGAAGGGGACUAAAA |
| | Dobrava-Begrade | | CAAUGAGCGUUUAUGCUUACGA |
| | orthohantavirus Segment M | | |
| | CasM.26 crRNA targeting | 1172 | GCCACCCCAAAAAUGAAGGGGACUAAAA |
| | Dobrava-Begrade | | CAGUAUGCUCCGCAAGAUAUAG |
| | orthohantavirus Segment M | | |
| | CasM.26 crRNA targeting | 1173 | GCCACCCCAAAAAUGAAGGGGACUAAAA |
| | Dobrava-Begrade | | CAGGGAAUCAGGUACCACAUCC |
| | orthohantavirus Segment L | | |
| | CasM.26 crRNA targeting | 1174 | GCCACCCCAAAAAUGAAGGGGACUAAAA |
| | Dobrava-Begrade | | CAGUCUACGUAAGAGAACUAUA |
| | orthohantavirus Segment L | | |
| | CasM.26 crRNA targeting | 1175 | GCCACCCCAAAAAUGAAGGGGACUAAAA |
| | Dobrava-Begrade | | CAGUAGUAUGCUCCGGAAAAUG |
| | orthohantavirus Segment L | | |
| | CasM.26 crRNA targeting | 1176 | GCCACCCCAAAAAUGAAGGGGACUAAAA |
| | Hantaan orthohantavirus | | CACCACCCACAACGGAUUAACU |
| | Segment S | | |
| | CasM.26 crRNA targeting | 1177 | GCCACCCCAAAAAUGAAGGGGACUAAAA |
| | Hantaan orthohantavirus | | CAGCUAAACCCACCCACAACGG |
| | Segment S | | |
| | CasM.26 crRNA targeting | 1178 | GCCACCCCAAAAAUGAAGGGGACUAAAA |
| | Hantaan orthohantavirus | | CAGGCUACUUAAGGUAGCCUGG |
| | Segment S | | |
| | CasM.26 crRNA targeting | 1179 | GCCACCCCAAAAAUGAAGGGGACUAAAA |
| | Hantaan orthohantavirus | | CACGGGACAGAGAAUGCUUAGU |
| | Segment M | | |
| | CasM.26 crRNA targeting | 1180 | GCCACCCCAAAAAUGAAGGGGACUAAAA |
| | Hantaan orthohantavirus | | CAGCUUCCUUACGGGACAGAGA |
| | Segment M | | |
| | CasM.26 crRNA targeting | 1181 | GCCACCCCAAAAAUGAAGGGGACUAAAA |
| | Hantaan orthohantavirus | | CAGUAGACACCGCAAGAUGUUA |
| | Segment M | | |
| | CasM.26 crRNA targeting | 1182 | GCCACCCCAAAAAUGAAGGGGACUAAAA |
| | Hantaan orthohantavirus | | CAUCUAUCGAGACUUUCGAACU |
| | Segment L | | |
| | CasM.26 crRNA targeting | 1183 | GCCACCCCAAAAAUGAAGGGGACUAAAA |
| | Hantaan orthohantavirus | | CACGAGACUUUCGAACUGGUCA |
| | Segment L | | |
| | CasM.26 crRNA targeting | 1184 | GCCACCCCAAAAAUGAAGGGGACUAAAA |
| | Hantaan orthohantavirus | | CAGUAGUAGUAUGCUCCGGAAA |
| | Segment L | | |
| | CasM.26 crRNA targeting | 1185 | GCCACCCCAAAAAUGAAGGGGACUAAAA |
| | Puumala orthohantavirus | | CAUCACAUUGUUGGGCCCACUA |
| | Segment S | | |
| | CasM.26 crRNA targeting | 1186 | GCCACCCCAAAAAUGAAGGGGACUAAAA |
| | Puumala orthohantavirus | | CACAUGUUGAGGUAGUAAAUUG |
| | Segment S | | |
| | CasM.26 crRNA targeting | 1187 | GCCACCCCAAAAAUGAAGGGGACUAAAA |
| | Puumala orthohantavirus | | CAGGCUGGUAACAAUAACAAGC |
| | Segment S | | |
| | CasM.26 crRNA targeting | 1188 | GCCACCCCAAAAAUGAAGGGGACUAAAA |
| | Puumala orthohantavirus | | CAUAGUAUGCUCCGCAGGAACA |
| | Segment M | | |
| | CasM.26 crRNA targeting | 1189 | GCCACCCCAAAAAUGAAGGGGACUAAAA |
| | Puumala orthohantavirus | | CAUUUGCAUAACUAGGCCUACG |
| | Segment M | | |
| | CasM.26 crRNA targeting | 1190 | GCCACCCCAAAAAUGAAGGGGACUAAAA |
| | Puumala orthohantavirus | | CAGGCAUAAUCGGUAUGGAGUA |
| | Segment M | | |
| | CasM.26 crRNA targeting | 1191 | GCCACCCCAAAAAUGAAGGGGACUAAAA |
| | Puumala orthohantavirus | | CAUAUGCUCCGAGAAAAGAGCG |
| | Segment L | | |
| | CasM.26 crRNA targeting | 1192 | GCCACCCCAAAAAUGAAGGGGACUAAAA |
| | Puumala orthohantavirus | | CAUAAGGAAUCGGGUAGCACAU |
| | Segment L | | |
| | CasM.26 crRNA targeting | 1193 | GCCACCCCAAAAAUGAAGGGGACUAAAA |
| | Puumala orthohantavirus | | CACGCUUUAACAAGAUAUCAGC |
| | Segment L | | |
| | CasM.26 crRNA targeting | 1194 | GCCACCCCAAAAAUGAAGGGGACUAAAA |
| | Seoul orthohantavirus Segment | | CAAUGGAAGUUGUCCACUGCCU |
| | S | | |
| | CasM.26 crRNA targeting | 1195 | GCCACCCCAAAAAUGAAGGGGACUAAAA |
| | Seoul orthohantavirus Segment | | CACCGAGAUGGAAGUUGUCCAC |
| | S | | |
| | CasM.26 crRNA targeting | 1196 | GCCACCCCAAAAAUGAAGGGGACUAAAA |
| | Seoul orthohantavirus Segment | | CAAUCACCGAGAUGGAAGUUGU |
| | S | | |
| | CasM.26 crRNA targeting | 1197 | GCCACCCCAAAAAUGAAGGGGACUAAAA |
| | Seoul orthohantavirus Segment | | CAAACCAAUUGCCUCGUACCUC |
| | L | | |
| | CasM.26 crRNA targeting | 1198 | GCCACCCCAAAAAUGAAGGGGACUAAAA |
| | Seoul orthohantavirus Segment | | CACCUGCAACCAAUUGCCUCGU |
| | L | | |
| | CasM.26 crRNA targeting | 1199 | GCCACCCCAAAAAUGAAGGGGACUAAAA |
| | Seoul orthohantavirus Segment | | CACGUACCUCUCCAUGAUGCCC |
| | L | | |
| | CasM.26 crRNA targeting | 1200 | GCCACCCCAAAAAUGAAGGGGACUAAAA |
| | Seoul orthohantavirus Segment | | CAGAUGUCAAGACAACACCCCG |
| | M | | |
| | CasM.26 crRNA targeting | 1201 | GCCACCCCAAAAAUGAAGGGGACUAAAA |
| | Seoul orthohantavirus Segment | | CACACUAUGGCCACCUUUCCCG |
| | M | | |
| | CasM.26 crRNA targeting | 1202 | GCCACCCCAAAAAUGAAGGGGACUAAAA |
| | Seoul orthohantavirus Segment | | CAGACAGUAUUUUGUCCUCGUG |
| | M | | |
| | CasM.26 crRNA targeting Sin | 1203 | GCCACCCCAAAAAUGAAGGGGACUAAAA |
| | Nombre orthohantavirus | | CAUAGUAUGCUCCUUGAAAAGC |
| | Segment S | | |
| | CasM.26 crRNA targeting Sin | 1204 | GCCACCCCAAAAAUGAAGGGGACUAAAA |
| | Nombre orthohantavirus | | CAGGUAGAUUGUUGUAAUGAAG |
| | Segment S | | |
| | CasM.26 crRNA targeting Sin | 1205 | GCCACCCCAAAAAUGAAGGGGACUAAAA |
| | Nombre orthohantavirus | | CAAAUGAGGUAGAUUGUUGUAA |
| | Segment S | | |
| | CasM.26 crRNA targeting Sin | 1188 | GCCACCCCAAAAAUGAAGGGGACUAAAA |
| | Nombre orthohantavirus | | CAUAGUAUGCUCCGCAGGAACA |
| | Segment M | | |
| | CasM.26 crRNA targeting Sin | 1206 | GCCACCCCAAAAAUGAAGGGGACUAAAA |
| | Nombre orthohantavirus | | CACCUCGUAAUAAGCAAAUGGG |
| | Segment M | | |
| | CasM.26 crRNA targeting Sin | 1207 | GCCACCCCAAAAAUGAAGGGGACUAAAA |
| | Nombre orthohantavirus | | CAGCUAAAAAGGAGUAUCGAUA |
| | Segment M | | |
| | CasM.26 crRNA targeting Sin | 1208 | GCCACCCCAAAAAUGAAGGGGACUAAAA |
| | Nombre orthohantavirus | | CACGGACCAACCGUUGUGCAUC |
| | Segment L | | |
| | CasM.26 crRNA targeting Sin | 1209 | GCCACCCCAAAAAUGAAGGGGACUAAAA |
| | Nombre orthohantavirus | | CAUUCUCUCGGACCAACCGUUG |
| | Segment L | | |
| | CasM.26 crRNA targeting Sin | 1210 | GCCACCCCAAAAAUGAAGGGGACUAAAA |
| | Nombre orthohantavirus | | CAUAACAACACAUCAACGUGCC |
| | Segment L | | |
| | CasM.26 crRNA targeting | 1211 | GCCACCCCAAAAAUGAAGGGGACUAAAA |
| | Lumpy Skin Disease Virus P32 | | CAUUGUCACUUUUUAAUUCUGG |
| | CasM.26 crRNA targeting | 1212 | GCCACCCCAAAAAUGAAGGGGACUAAAA |
| | Lumpy Skin Disease Virus P32 | | CAGCCUCUUUUGUACAAAGAGC |
| | CasM.26 crRNA targeting | 1213 | GCCACCCCAAAAAUGAAGGGGACUAAAA |
| | Lumpy Skin Disease Virus P32 | | CACUUUUUGCCUCUUUUGUACA |
| | CasM.26 crRNA targeting | 1214 | GCCACCCCAAAAAUGAAGGGGACUAAAA |
| | Mycobacterium bovis 23S | | CAACCGAACGUGGCUAUCCAGC |
| | CasM.26 crRNA targeting | 1215 | GCCACCCCAAAAAUGAAGGGGACUAAAA |
| | Mycobacterium bovis 23S | | CAAGUUUUCGGCCGGUUAGCAC |
| | CasM.26 crRNA targeting | 1216 | GCCACCCCAAAAAUGAAGGGGACUAAAA |
| | Mycobacterium bovis 23S | | CAUCAGCGGUUAUCCUGACCGA |
| | CasM.26 crRNA targeting | 1217 | GCCACCCCAAAAAUGAAGGGGACUAAAA |
| | Escherichia coli 23S | | CACUGUAUCGCGCGCCUUUCCA |
| | CasM.26 crRNA targeting | 1218 | GCCACCCCAAAAAUGAAGGGGACUAAAA |
| | Escherichia coli 23S | | CAUGUCACCCUGUAUCGCGCGC |
| | CasM.26 crRNA targeting | 1219 | GCCACCCCAAAAAUGAAGGGGACUAAAA |
| | Escherichia coli 23S | | CAGGCUGUCACCCUGUAUCGCG |
| | CasM.26 crRNA targeting | 1220 | GCCACCCCAAAAAUGAAGGGGACUAAAA |
| | Cryptococcus neoformans | | CAUACAGUAGUUAGCGCUAUGA |
| | CasM.26 crRNA targeting | 1221 | GCCACCCCAAAAAUGAAGGGGACUAAAA |
| | Cryptococcus neoformans | | CAGUCUUUUGUCACGAGGAUAA |
| | CasM.26 crRNA targeting | 1222 | GCCACCCCAAAAAUGAAGGGGACUAAAA |
| | Cryptococcus neoformans | | CAGCUAUGCAUCCAUGCAUCGC |
| | Cas12 Variant crRNA targeting | 1223 | UAAUUUCUACUAAGUGUAGAUGUCCGAG |
| | Aspergillus 18S | | CGUCAUUGCUGCC |
| | Cas12 Variant crRNA targeting | 1224 | UAAUUUCUACUAAGUGUAGAUGUUCUUC |
| | Aspergillus 18S | | AUCGAUGCCGGAA |
| | Cas12 Variant crRNA targeting | 1225 | UAAUUUCUACUAAGUGUAGAUAGGGCA |
| | Aspergillus 18S | | GCAAUGACGCUCGG |
| | Cas12 Variant crRNA targeting | 1226 | UAAUUUCUACUAAGUGUAGAUUUGUGC |
| | Aspergillus 28S | | GCUAUCGGUCUCCG |
| | Cas12 Variant crRNA targeting | 1227 | UAAUUUCUACUAAGUGUAGAUACAACUC |
| | Aspergillus 28S | | GACUCGUCGAAGG |
| | Cas12 Variant crRNA targeting | 1228 | UAAUUUCUACUAAGUGUAGAUGCCGAAG |
| | Aspergillus 28S | | CGCGUUCCUCGGU |
| | Cas12 Variant crRNA targeting | 1229 | UAAUUUCUACUAAGUGUAGAUUUAUCGC |
| | Aspergillus ITS | | AUUUCGCUGCGUU |
| | Cas12 Variant crRNA targeting | 1230 | UAAUUUCUACUAAGUGUAGAUAAUACCA |
| | Aspergillus ITS | | GGGGGCGCAAUGU |
| | Cas12 Variant crRNA targeting | 1231 | UAAUUUCUACUAAGUGUAGAUCGCAUUU |
| | Aspergillus ITS | | CGCUGCGUUCUUC |
| | Cas12 Variant crRNA targeting | 1232 | UAAUUUCUACUAAGUGUAGAUAGGAUU |
| | Aspergillus flavus | | CGAACCUAUAAUAU |
| | Mitochondrion | | |
| | Cas12 Variant crRNA targeting | 1233 | UAAUUUCUACUAAGUGUAGAUAAUAUU |
| | Aspergillus flavus | | AACAGUAUUCCGCU |
| | Mitochondrion | | |
| | Cas12 Variant crRNA targeting | 1234 | UAAUUUCUACUAAGUGUAGAUUUAAGG |
| | Aspergillus flavus | | CUUACAGGAUUCGA |
| | Mitochondrion | | |
| | Cas12 Variant crRNA targeting | 1235 | UAAUUUCUACUAAGUGUAGAUGUAAGG |
| | Aspergillus fumigatus | | UAACCUUAACUAAU |
| | Mitochondrion | | |
| | Cas12 Variant crRNA targeting | 1236 | UAAUUUCUACUAAGUGUAGAUUCAAUG |
| | Aspergillus fumigatus | | GAAGGUUCUGUUAC |
| | Mitochondrion | | |
| | Cas12 Variant crRNA targeting | 1237 | UAAUUUCUACUAAGUGUAGAUUUUGUA |
| | Aspergillus fumigatus | | UAUAAAGAGGCGAC |
| | Mitochondrion | | |
| | Cas12 Variant crRNA targeting | 1238 | UAAUUUCUACUAAGUGUAGAUUCAUCGA |
| | Candida ITS | | UGCGAGAACCAAG |
| | Cas12 Variant crRNA targeting | 1239 | UAAUUUCUACUAAGUGUAGAUCUUGGU |
| | Candida ITS | | UCUCGCAUCGAUGA |
| | Cas12 Variant crRNA targeting | 1240 | UAAUUUCUACUAAGUGUAGAUUCGAUGC |
| | Candida ITS | | GAGAACCAAGAGA |
| | Cas12 Variant crRNA targeting | 1241 | UAAUUUCUACUAAGUGUAGAUCCUUGCG |
| | Candida 18S | | ACCAUACUCCCCC |
| | Cas12 Variant crRNA targeting | 1242 | UAAUUUCUACUAAGUGUAGAUGGUUAA |
| | Candida 18S | | GACUACGACGGUAU |
| | Cas12 Variant crRNA targeting | 1243 | UAAUUUCUACUAAGUGUAGAUGUCAAA |
| | Candida 18S | | UUAAGCCGCAGGCU |
| | Cas12 Variant crRNA targeting | 1244 | UAAUUUCUACUAAGUGUAGAUUUCGCUA |
| | Candida 28S | | UCGGUCUCUCGCC |
| | Cas12 Variant crRNA targeting | 1245 | UAAUUUCUACUAAGUGUAGAUCCCUUUC |
| | Candida 28S | | AACAAUUUCACGU |
| | Cas12 Variant crRNA targeting | 1246 | UAAUUUCUACUAAGUGUAGAUAUUCCCA |
| | Candida 28S | | AACAACUCGACUC |
| | Cas12 Variant crRNA targeting | 1247 | UAAUUUCUACUAAGUGUAGAUCGACAAC |
| | Candida Mitochondrion | | AAUGUAACGCCUG |
| | Cas12 Variant crRNA targeting | 1248 | UAAUUUCUACUAAGUGUAGAUGGGGAC |
| | Candida Mitochondrion | | UUGCCGAUUAGUCA |
| | Cas12 Variant crRNA targeting | 1249 | UAAUUUCUACUAAGUGUAGAUUGAUGA |
| | Candida Mitochondrion | | GUGAUUAGUGCGAA |
| | Cas12 Variant crRNA targeting | 1250 | UAAUUUCUACUAAGUGUAGAUACGGGG |
| | Candida albicans ERG11 | | UUGCCAAUGUUAUG |
| | Cas12 Variant crRNA targeting | 1251 | UAAUUUCUACUAAGUGUAGAUACCUCAU |
| | Candida albicans ERG11 | | UAUUGGAGACGUG |
| | Cas12 Variant crRNA targeting | 1252 | UAAUUUCUACUAAGUGUAGAUAACUACU |
| | Candida albicans ERG11 | | CCAGUUUUCGGUA |
| | Cas12 Variant crRNA targeting | 1253 | UAAUUUCUACUAAGUGUAGAUAGUUUU |
| | Candida lusitaniae ERG9 | | GGCUCGUUUCGCCG |
| | Cas12 Variant crRNA targeting | 1254 | UAAUUUCUACUAAGUGUAGAUGUCGUU |
| | Candida lusitaniae ERG9 | | AAUGCACUCGACAC |
| | Cas12 Variant crRNA targeting | 1255 | UAAUUUCUACUAAGUGUAGAUACGACUU |
| | Candida lusitaniae ERG9 | | GGUUUUGAACGCG |
| | Cas12 Variant crRNA targeting | 1256 | UAAUUUCUACUAAGUGUAGAUGGUGCCG |
| | Clostridium difficile 16S | | CACUAACGCAUUA |
| | Cas12 Variant crRNA targeting | 1257 | UAAUUUCUACUAAGUGUAGAUGUACAG |
| | Clostridium difficile 16S | | GAUGGACCCGCGUC |
| | Cas12 Variant crRNA targeting | 1258 | UAAUUUCUACUAAGUGUAGAUUAAUCG |
| | Clostridium difficile 16S | | GAGAGUUCCCUUCG |
| | Cas12 Variant crRNA targeting | 1259 | UAAUUUCUACUAAGUGUAGAUCGGGAA |
| | Clostridium perfringens 16S | | ACGGAUUAGCGGCG |
| | Cas12 Variant crRNA targeting | 1260 | UAAUUUCUACUAAGUGUAGAUAAUCGA |
| | Clostridium perfringens 16S | | GGAAAUCCCUUCGG |
| | Cas12 Variant crRNA targeting | 1261 | UAAUUUCUACUAAGUGUAGAUCUAACGC |
| | Clostridium perfringens 16S | | AUUAAGUAUUCCG |
| | Cas12 Variant crRNA targeting | 1262 | UAAUUUCUACUAAGUGUAGAUAGGCGAC |
| | Clostridium tetani 16S | | GUAGGGUAGCCGA |
| | Cas12 Variant crRNA targeting | 1263 | UAAUUUCUACUAAGUGUAGAUAAGAGG |
| | Clostridium tetani 16S | | GGAAUAGCCCUCCG |
| | Cas12 Variant crRNA targeting | 1264 | UAAUUUCUACUAAGUGUAGAUAGAUGG |
| | Clostridium tetani 16S | | GCCCGCGCCCCAUU |
| | Cas12 Variant crRNA targeting | 1265 | UAAUUUCUACUAAGUGUAGAUUCGCGUC |
| | Corynebacterium 16S | | GUCUGUGAAAUUC |
| | Cas12 Variant crRNA targeting | 1266 | UAAUUUCUACUAAGUGUAGAUGUUUUA |
| | Corynebacterium 16S | | GCCUUGCGGCCGUA |
| | Cas12 Variant crRNA targeting | 941 | UAAUUUCUACUAAGUGUAGAUUCUGCGA |
| | Corynebacterium 16S | | UUACUAGCGACUC |
| | Cas12 Variant crRNA targeting | 1267 | UAAUUUCUACUAAGUGUAGAUAGACGU |
| | Corynebacterium rpoB | | UCACGCUUCUCACU |
| | Cas12 Variant crRNA targeting | 1268 | UAAUUUCUACUAAGUGUAGAUAGUGAG |
| | Corynebacterium rpoB | | AAGCGUGAACGUCU |
| | Cas12 Variant crRNA targeting | 1269 | UAAUUUCUACUAAGUGUAGAUGAGCGCA |
| | Corynebacterium rpoB | | CCAACCAGGGCAC |
| | Cas12 Variant crRNA targeting | 1270 | UAAUUUCUACUAAGUGUAGAUAGAGUA |
| | Corynebacterium tox | | UCCCGCAGCGUCGU |
| | Cas12 Variant crRNA targeting | 1271 | UAAUUUCUACUAAGUGUAGAUACAAUA |
| | Corynebacterium tox | | AAUACGACGCUGCG |
| | Cas12 Variant crRNA targeting | 1272 | UAAUUUCUACUAAGUGUAGAUUACAUCG |
| | Corynebacterium tox | | CAUCUUGGCCACG |
| | Cas12 Variant crRNA targeting | 1273 | UAAUUUCUACUAAGUGUAGAUCGGCGAA |
| | Corynebacterium diphtheriae | | CUAAUCAAUGCAC |
| | chrA | | |
| | Cas12 Variant crRNA targeting | 1274 | UAAUUUCUACUAAGUGUAGAUCCGCAGG |
| | Corynebacterium diphtheriae | | UCGGAGAUAUAAG |
| | chrA | | |
| | Cas12 Variant crRNA targeting | 1275 | UAAUUUCUACUAAGUGUAGAUAACGGCC |
| | Corynebacterium diphtheriae | | GGUUUGGAAGCGA |
| | chrA | | |
| | Cas12 Variant crRNA targeting | 1276 | UAAUUUCUACUAAGUGUAGAUGGACGCG |
| | Corynebacterium | | CGAGUUAAUGAUC |
| | pseudotuberculosis pld | | |
| | Cas12 Variant crRNA targeting | 1277 | UAAUUUCUACUAAGUGUAGAUCGUAAG |
| | Corynebacterium | | UCCAGCGAAGCUCG |
| | pseudotuberculosis pld | | |
| | Cas12 Variant crRNA targeting | 1278 | UAAUUUCUACUAAGUGUAGAUAAGAUC |
| | Corynebacterium | | AUUAACUCGCGCGU |
| | pseudotuberculosis pld | | |
| | Cas12 Variant crRNA targeting | 1279 | UAAUUUCUACUAAGUGUAGAUCGCGUAG |
| | Corynebacterium | | UUGCGAUGCGGUG |
| | striatum chrA | | |
| | Cas12 Variant crRNA targeting | 1280 | UAAUUUCUACUAAGUGUAGAUGCAGUCC |
| | Corynebacterium | | CCGACGCGAUAAA |
| | striatum chrA | | |
| | Cas12 Variant crRNA targeting | 1281 | UAAUUUCUACUAAGUGUAGAUGACGCGA |
| | Corynebacterium | | UAAAACGCAACCU |
| | striatum chrA | | |
| | Cas12 Variant crRNA targeting | 1282 | UAAUUUCUACUAAGUGUAGAUGCGGACC |
| | Corynebacterium ulcerans pld | | GACGCAUGACGUG |
| | Cas12 Variant crRNA targeting | 1283 | UAAUUUCUACUAAGUGUAGAUAGCGGAC |
| | Corynebacterium ulcerans pld | | CGACGCAUGACGU |
| | Cas12 Variant crRNA targeting | 1284 | UAAUUUCUACUAAGUGUAGAUCGCGACU |
| | Corynebacterium ulcerans pld | | UAGCACGCAAAUA |
| | Cas12 Variant crRNA targeting | 1285 | UAAUUUCUACUAAGUGUAGAUCUCGCGG |
| | Campylobacteraceae 16S | | UAUUGCGUCUCAU |
| | Cas12 Variant crRNA targeting | 1286 | UAAUUUCUACUAAGUGUAGAUGCUCGUU |
| | Campylobacteraceae 16S | | GCGGGACUUAACC |
| | Cas12 Variant crRNA targeting | 1287 | UAAUUUCUACUAAGUGUAGAUGACCGUA |
| | Campylobacteraceae 16S | | CUCCCCAGGCGGU |
| | Cas12 Variant crRNA targeting | 1288 | UAAUUUCUACUAAGUGUAGAUGCGUCGA |
| | Campylobacteraceae 23S | | AGGUAUACCGGUA |
| | Cas12 Variant crRNA targeting | 1289 | UAAUUUCUACUAAGUGUAGAUGACUGCG |
| | Campylobacteraceae 23S | | AUAAGCUACGGGG |
| | Cas12 Variant crRNA targeting | 1290 | UAAUUUCUACUAAGUGUAGAUAUGUUG |
| | Campylobacteraceae 23S | | CCCGUACCGUAAAC |
| | Cas12 Variant crRNA targeting | 1291 | UAAUUUCUACUAAGUGUAGAUACGGUAC |
| | Campylobacteraceae flaG | | CUAAGGAAUAAGC |
| | Cas12 Variant crRNA targeting | 1292 | UAAUUUCUACUAAGUGUAGAUAAUAAG |
| | Campylobacteraceae flaG | | UGAUCGAUUGAGCC |
| | Cas12 Variant crRNA targeting | 1293 | UAAUUUCUACUAAGUGUAGAUGAAGUG |
| | Campylobacteraceae flaG | | GAUUAGUGGCGCAC |
| | Cas12 Variant crRNA targeting | 1294 | UAAUUUCUACUAAGUGUAGAUCUGUAUC |
| | Escherichia coli 23S | | GCGCGCCUUUCCA |
| | Cas12 Variant crRNA targeting | 1295 | UAAUUUCUACUAAGUGUAGAUUAUCGCG |
| | Escherichia coli 23S | | CGCCUUUCCAGAC |
| | Cas12 Variant crRNA targeting | 1296 | UAAUUUCUACUAAGUGUAGAUUGUCACC |
| | Escherichia coli 23S | | CUGUAUCGCGCGC |
| | Cas12 Variant crRNA targeting | 1297 | UAAUUUCUACUAAGUGUAGAUAUCGUA |
| | Escherichia coli O157:H1 | | UACGCGCUGUAUCU |
| | pO157 | | |
| | Cas12 Variant crRNA targeting | 1298 | UAAUUUCUACUAAGUGUAGAUGCCAUCG |
| | Escherichia coli O157:H1 | | CGUUGUCAUACGA |
| | pO157 | | |
| | Cas12 Variant crRNA targeting | 1299 | UAAUUUCUACUAAGUGUAGAUGUCGCUC |
| | Escherichia coli O157:H1 | | CGUAUAGGCCAGU |
| | pO157 | | |
| | Cas12 Variant crRNA targeting | 1300 | UAAUUUCUACUAAGUGUAGAUAGCCAUC |
| | Escherichia coli O157:H1 | | GCGUUGUCAUACG |
| | stx2A | | |
| | Cas12 Variant crRNA targeting | 1297 | UAAUUUCUACUAAGUGUAGAUAUCGUA |
| | Escherichia coli O157:H1 | | UACGCGCUGUAUCU |
| | stx2A | | |
| | Cas12 Variant crRNA targeting | 1299 | UAAUUUCUACUAAGUGUAGAUGUCGCUC |
| | Escherichia coli O157:H1 | | CGUAUAGGCCAGU |
| | stx2A | | |
| | Cas12 Variant crRNA targeting | 1301 | UAAUUUCUACUAAGUGUAGAUCGGAGCG |
| | Shigella Virulence Factor | | UUGUCGUAACAGU |
| | mxiM | | |
| | Cas12 Variant crRNA targeting | 1302 | UAAUUUCUACUAAGUGUAGAUGCGUUG |
| | Shigella Virulence Factor | | GGUGUGGAUCACUA |
| | mxiM | | |
| | Cas12 Variant crRNA targeting | 1303 | UAAUUUCUACUAAGUGUAGAUCUCUUAA |
| | Shigella Virulence Factor | | AACGAUAGUGGCU |
| | mxiM | | |
| | Cas12 Variant crRNA targeting | 1304 | UAAUUUCUACUAAGUGUAGAUAAAAAC |
| | Shigella Virulence Factor | | UUCGCGCAAAAACC |
| | ospC1 | | |
| | Cas12 Variant crRNA targeting | 1305 | UAAUUUCUACUAAGUGUAGAUUGAUCA |
| | Shigella Virulence Factor | | UGACUCUCCAUAUG |
| | ospC1 | | |
| | Cas12 Variant crRNA targeting | 1306 | UAAUUUCUACUAAGUGUAGAUUCCACCU |
| | Shigella Virulence Factor | | GUUUUUUACCAUG |
| | ospC1 | | |
| | Cas12 Variant crRNA targeting | 1307 | UAAUUUCUACUAAGUGUAGAUAAUAGC |
| | Shiga Toxin stx | | GCGGUUUGACUUAC |
| | Cas12 Variant crRNA targeting | 1308 | UAAUUUCUACUAAGUGUAGAUGUGCGAC |
| | Shiga Toxin stx | | CCCAAUAGCGCGG |
| | Cas12 Variant crRNA targeting | 1309 | UAAUUUCUACUAAGUGUAGAUGGUUAA |
| | Shiga Toxin stx | | CCCAAUCGGCUUCA |
| | Cas12 Variant crRNA targeting | 1310 | UAAUUUCUACUAAGUGUAGAUACGGGG |
| | Listeria monocytogenes 16S | | GCCGCACAAGCGGU |
| | Cas12 Variant crRNA targeting | 1311 | UAAUUUCUACUAAGUGUAGAUGGGAUA |
| | Listeria monocytogenes 16S | | ACUCCGGGAAACCG |
| | Cas12 Variant crRNA targeting | 1312 | UAAUUUCUACUAAGUGUAGAUAACGAAC |
| | Listeria monocytogenes 16S | | GGAGGAAGAGCUU |
| | Cas12 Variant crRNA targeting | 1313 | UAAUUUCUACUAAGUGUAGAUAGUCGG |
| | Listeria monocytogenes 23S | | AUAGUAUCCUUACG |
| | Cas12 Variant crRNA targeting | 1314 | UAAUUUCUACUAAGUGUAGAUAGCGGA |
| | Listeria monocytogenes 23S | | AAAAGCGGAGCCGU |
| | Cas12 Variant crRNA targeting | 1315 | UAAUUUCUACUAAGUGUAGAUAAGACG |
| | Listeria monocytogenes 23S | | GUAACAGCCCGGUA |
| | Cas12 Variant crRNA targeting | 1316 | UAAUUUCUACUAAGUGUAGAUAUCUACC |
| | Listeria monocytogenes prfA | | GGAUCUACCGGAU |
| | Cas12 Variant crRNA targeting | 1317 | UAAUUUCUACUAAGUGUAGAUGUAGAU |
| | Listeria monocytogenes prfA | | CCGGUAGAUCCGGU |
| | Cas12 Variant crRNA targeting | 1318 | UAAUUUCUACUAAGUGUAGAUGCUUAAC |
| | Listeria monocytogenes prfA | | CGGGGAGGGUCAU |
| | Cas12 Variant crRNA targeting | 1319 | UAAUUUCUACUAAGUGUAGAUACAACGC |
| | Mycobacterium bovis 16S | | UCGCACCCUACGU |
| | Cas12 Variant crRNA targeting | 1320 | UAAUUUCUACUAAGUGUAGAUCGUCGCC |
| | Mycobacterium bovis 16S | | UUGGUAGGCCGUC |
| | Cas12 Variant crRNA targeting | 1321 | UAAUUUCUACUAAGUGUAGAUCGUCAAU |
| | Mycobacterium bovis 16S | | CCGAGAGAACCCG |
| | Cas12 Variant crRNA targeting | 1322 | UAAUUUCUACUAAGUGUAGAUGAUGACC |
| | Mycobacterium bovis 23S | | GAUAGCGGAUUAG |
| | Cas12 Variant crRNA targeting | 1323 | UAAUUUCUACUAAGUGUAGAUUCAACCG |
| | Mycobacterium bovis 23S | | AGCGUGGAUCCGA |
| | Cas12 Variant crRNA targeting | 1324 | UAAUUUCUACUAAGUGUAGAUGCACGAG |
| | Mycobacterium bovis 23S | | UGAUGUCGUGCUA |
| | Cas12 Variant crRNA targeting | 1325 | UAAUUUCUACUAAGUGUAGAUAGCGGCC |
| | Neisseria meningitidis 16S | | GAUAUCUGAUUAG |
| | Cas12 Variant crRNA targeting | 1326 | UAAUUUCUACUAAGUGUAGAUUCGGGCC |
| | Neisseria meningitidis 16S | | UUGCGCUAUUCGA |
| | Cas12 Variant crRNA targeting | 1327 | UAAUUUCUACUAAGUGUAGAUACAUGU |
| | Neisseria meningitidis 16S | | ACGGAAUCCUCCGG |
| | Cas12 Variant crRNA targeting | 1328 | UAAUUUCUACUAAGUGUAGAUUACGUA |
| | Neisseria meningitidis 23S | | AUAACUGUCGAGAU |
| | Cas12 Variant crRNA targeting | 1329 | UAAUUUCUACUAAGUGUAGAUCAACAGC |
| | Neisseria meningitidis 23S | | GGUACUAAGCGUA |
| | Cas12 Variant crRNA targeting | 1330 | UAAUUUCUACUAAGUGUAGAUACAGCGG |
| | Neisseria meningitidis 23S | | UACUAAGCGUACG |
| | Cas12 Variant crRNA targeting | 1331 | UAAUUUCUACUAAGUGUAGAUCGUACAC |
| | Salmonella enterica Paratyphi | | AAAAGCGCAUGUG |
| | 23S | | |
| | Cas12 Variant crRNA targeting | 1332 | UAAUUUCUACUAAGUGUAGAUUACACAA |
| | Salmonella enterica Paratyphi | | AAGCGCAUGUGCU |
| | 23S | | |
| | Cas12 Variant crRNA targeting | 1333 | UAAUUUCUACUAAGUGUAGAUAGAGCCU |
| | Salmonella enterica Paratyphi | | GAAUCAGCAUGUG |
| | 23S | | |
| | Cas12 Variant crRNA targeting | 1334 | UAAUUUCUACUAAGUGUAGAUUCGCGUA |
| | Salmonella enterica Paratyphi | | GUCGGAAUCUUCG |
| | filC | | |
| | Cas12 Variant crRNA targeting | 1335 | UAAUUUCUACUAAGUGUAGAUAUACACG |
| | Salmonella enterica Paratyphi | | GUCGAUUUCGUUC |
| | filC | | |
| | Cas12 Variant crRNA targeting | 1336 | UAAUUUCUACUAAGUGUAGAUAACGGU |
| | Salmonella enterica Paratyphi | | UCUGAACCGCACCC |
| | filC | | |
| | Cas12 Variant crRNA targeting | 1337 | UAAUUUCUACUAAGUGUAGAUGAUACG |
| | Salmonella enterica Typhi | | GGAUCGAUAAUUGC |
| | segment 2/20 | | |
| | Cas12 Variant crRNA targeting | 1338 | UAAUUUCUACUAAGUGUAGAUCCGUAUU |
| | Salmonella enterica Typhi | | CUUACCCACCGUA |
| | segment 2/20 | | |
| | Cas12 Variant crRNA targeting | 1339 | UAAUUUCUACUAAGUGUAGAUCGCCGUA |
| | Salmonella enterica Typhi | | UUCUUACCCACCG |
| | segment 2/20 | | |
| | Cas12 Variant crRNA targeting | 1340 | UAAUUUCUACUAAGUGUAGAUACGGCAG |
| | Salmonella enterica Typhi | | CACCCACGCGGCA |
| | segment | | |
| | Cas12 Variant crRNA targeting | 1341 | UAAUUUCUACUAAGUGUAGAUGGCUACA |
| | Salmonella enterica Typhi | | GGAGCUGUCGGUA |
| | segment | | |
| | Cas12 Variant crRNA targeting | 1342 | UAAUUUCUACUAAGUGUAGAUUACUCCC |
| | Salmonella enterica Typhi | | UCUUUACGCGCAG |
| | segment | | |
| | Cas12 Variant crRNA targeting | 1343 | UAAUUUCUACUAAGUGUAGAUUAGUAG |
| | Salmonella enterica | | CGAAUGCUGAAGUU |
| | Typhimurium section 43/220 | | |
| | Cas12 Variant crRNA targeting | 1344 | UAAUUUCUACUAAGUGUAGAUGUAGCG |
| | Salmonella enterica | | AAUGCUGAAGUUGA |
| | Typhimurium section 43/220 | | |
| | Cas12 Variant crRNA targeting | 1345 | UAAUUUCUACUAAGUGUAGAUAAGUUG |
| | Salmonella enterica | | AUAACCCACUAAGU |
| | Typhimurium section 43/220 | | |
| | Cas12 Variant crRNA targeting | 1346 | UAAUUUCUACUAAGUGUAGAUUAAGCG |
| | Salmonella enterica | | AAGCGCCAUCAGGC |
| | Typhimurium section 217/220 | | |
| | Cas12 Variant crRNA targeting | 1347 | UAAUUUCUACUAAGUGUAGAUGCGAAUC |
| | Salmonella enterica | | AAUACAGAAUGCC |
| | Typhimurium section 217/220 | | |
| | Cas12 Variant crRNA targeting | 1348 | UAAUUUCUACUAAGUGUAGAUAUUGUU |
| | Salmonella enterica | | AGCGAAUCAAUACA |
| | Typhimurium section 217/220 | | |
| | Cas12 Variant crRNA targeting | 1349 | UAAUUUCUACUAAGUGUAGAUCGUUACA |
| | Salmonella enterica | | AUAAGCCUGGUGG |
| | Typhimurium section 99/220 | | |
| | Cas12 Variant crRNA targeting | 1350 | UAAUUUCUACUAAGUGUAGAUCCCACGU |
| | Salmonella enterica | | UACAAUAAGCCUG |
| | Typhimurium section 99/220 | | |
| | Cas12 Variant crRNA targeting | 1351 | UAAUUUCUACUAAGUGUAGAUCACGUUA |
| | Salmonella enterica | | CAAUAAGCCUGGU |
| | Typhimurium section 99/220 | | |
| | Cas12 Variant crRNA targeting | 1352 | UAAUUUCUACUAAGUGUAGAUUCACUCC |
| | Salmonella enterica Enteritides | | CUCUGGCCGCAGA |
| | difference region 1 | | |
| | Cas12 Variant crRNA targeting | 1353 | UAAUUUCUACUAAGUGUAGAUGGCAGCC |
| | Salmonella enterica Enteritides | | GUCACUCCCUCUG |
| | difference region 1 | | |
| | Cas12 Variant crRNA targeting | 1354 | UAAUUUCUACUAAGUGUAGAUGCCCGGC |
| | Salmonella enterica Enteritides | | AGCCGUCACUCCC |
| | difference region 1 | | |
| | Cas12 Variant crRNA targeting | 1355 | UAAUUUCUACUAAGUGUAGAUGGCAUUC |
| | Salmonella enterica Enteritides | | UGGGAUCAGAUAC |
| | caiC | | |
| | Cas12 Variant crRNA targeting | 1356 | UAAUUUCUACUAAGUGUAGAUGGGCAU |
| | Salmonella enterica Enteritides | | UCUGGGAUCAGAUA |
| | caiC | | |
| | Cas12 Variant crRNA targeting | 1357 | UAAUUUCUACUAAGUGUAGAUGAUACG |
| | Salmonella enterica Enteritides | | UAAAUAUCAGGCGA |
| | caiC | | |
| | Cas12 Variant crRNA targeting | 1358 | UAAUUUCUACUAAGUGUAGAUGACGUA |
| | Salmonella enterica Enteritides | | GGCGCGAGCUUCUU |
| | acrR | | |
| | Cas12 Variant crRNA targeting | 1359 | UAAUUUCUACUAAGUGUAGAUAUCAAAC |
| | Salmonella enterica Enteritides | | GAUUGCGGAGCAA |
| | acrR | | |
| | Cas12 Variant crRNA targeting | 1360 | UAAUUUCUACUAAGUGUAGAUCAGGGG |
| | Salmonella enterica Enteritides | | GAGCCGUUGAACGU |
| | acrR | | |
| | Cas12 Variant crRNA targeting | 1361 | UAAUUUCUACUAAGUGUAGAUGUUUUU |
| | Salmonella enterica Enteritides | | UAACUCGCCGCCAG |
| | citT | | |
| | Cas12 Variant crRNA targeting | 1362 | UAAUUUCUACUAAGUGUAGAUCGGUCAU |
| | Salmonella enterica Enteritides | | CAAGAACCUGCCA |
| | citT | | |
| | Cas12 Variant crRNA targeting | 1363 | UAAUUUCUACUAAGUGUAGAUGCCACCG |
| | Salmonella enterica Enteritides | | CUGUUUAAAUCGU |
| | citT | | |
| | Cas12 Variant crRNA targeting | 1364 | UAAUUUCUACUAAGUGUAGAUAAUACG |
| | Yersinia enterocolitica 16S | | UUGUUGGAUUGACG |
| | Cas12 Variant crRNA targeting | 1365 | UAAUUUCUACUAAGUGUAGAUGACCAAA |
| | Yersinia enterocolitica 16S | | GUGGGGGACCUUA |
| | Cas12 Variant crRNA targeting | 1366 | UAAUUUCUACUAAGUGUAGAUUUGGAU |
| | Yersinia enterocolitica 16S | | UGACGUUACUCGCA |
| | Cas12 Variant crRNA targeting | 1367 | UAAUUUCUACUAAGUGUAGAUAUAUGA |
| | Yersinia enterocolitica 23S | | AGCGUUAUAACCGA |
| | Cas12 Variant crRNA targeting | 1368 | UAAUUUCUACUAAGUGUAGAUUUAGAU |
| | Yersinia enterocolitica 23S | | GAAUACAUAGUCUA |
| | Cas12 Variant crRNA targeting | 1369 | UAAUUUCUACUAAGUGUAGAUAGAGUC |
| | Yersinia enterocolitica 23S | | UGAAUCAGUUUGUG |
| | Cas12 Variant crRNA targeting | 1370 | UAAUUUCUACUAAGUGUAGAUCUGGAA |
| | Yersinia pseudotuberculosis | | ACGGUGGCUAAUAC |
| | 16S | | |
| | Cas12 Variant crRNA targeting | 1371 | UAAUUUCUACUAAGUGUAGAUGAAACG |
| | Yersinia pseudotuberculosis | | GUGGCUAAUACCGC |
| | 16S | | |
| | Cas12 Variant crRNA targeting | 1372 | UAAUUUCUACUAAGUGUAGAUGGCCUUG |
| | Vibrio cholerae 16S | | CGCUAUCGGAUAU |
| | Cas12 Variant crRNA targeting | 1373 | UAAUUUCUACUAAGUGUAGAUGGAAAU |
| | Vibrio cholerae 16S | | UGCCCGGUAGAGGG |
| | Cas12 Variant crRNA targeting | 1374 | UAAUUUCUACUAAGUGUAGAUUGUUUG |
| | Vibrio cholerae 16S | | CCAGCACGUAAUGG |
| | Cas12 Variant crRNA targeting | 1375 | UAAUUUCUACUAAGUGUAGAUUGUCGCG |
| | Vibrio cholerae hlyA | | GACUCUUAACCGU |
| | Cas12 Variant crRNA targeting | 1376 | UAAUUUCUACUAAGUGUAGAUAUUGAG |
| | Vibrio cholerae hlyA | | CCGCGGUGGUUACG |
| | Cas12 Variant crRNA targeting | 1377 | UAAUUUCUACUAAGUGUAGAUAGAGGG |
| | Vibrio cholerae hlyA | | ACUGUCAACGCGCG |
| | Cas12 Variant crRNA targeting | 1378 | UAAUUUCUACUAAGUGUAGAUAUAGCG |
| | Vibrio parahaemolyticus 16S | | UAUUCGUUUGACGU |
| | Cas12 Variant crRNA targeting | 1379 | UAAUUUCUACUAAGUGUAGAUAUACCGC |
| | Vibrio parahaemolyticus 16S | | AUGAUGCCUACGG |
| | Cas12 Variant crRNA targeting | 1380 | UAAUUUCUACUAAGUGUAGAUAAACUG |
| | Vibrio parahaemolyticus 16S | | GCAGACUAGAGUGC |
| | Cas12 Variant crRNA targeting | 1381 | UAAUUUCUACUAAGUGUAGAUAGAGAA |
| | Vibrio vulnificus 16S | | UCUAGCGGAGACGC |
| | Cas12 Variant crRNA targeting | 1382 | UAAUUUCUACUAAGUGUAGAUUGGCUU |
| | Vibrio vulnificus 16S | | UCGGAGCUAACGCG |
| | Cas12 Variant crRNA targeting | 1383 | UAAUUUCUACUAAGUGUAGAUUCGGGCC |
| | Vibrio vulnificus 16S | | UCUCGCGUCAGGA |
| | Cas12 Variant crRNA targeting | 1384 | UAAUUUCUACUAAGUGUAGAUGGCGCCA |
| | Vibrio vulnificus 23S | | UAGUCUCUGGAUA |
| | Cas12 Variant crRNA targeting | 1385 | UAAUUUCUACUAAGUGUAGAUCGUAGU |
| | Vibrio vulnificus 23S | | UGACGAUGCAUGUU |
| | Cas12 Variant crRNA targeting | 1386 | UAAUUUCUACUAAGUGUAGAUAAUGAA |
| | Vibrio vulnificus 23S | | GCGAACCGGGAGAA |
| | Cas12 Variant crRNA targeting | 1387 | UAAUUUCUACUAAGUGUAGAUCGCGUAG |
| | Cytomegalovirus complete | | AAUCCGUACGGCC |
| | genome | | |
| | Cas12 Variant crRNA targeting | 1388 | UAAUUUCUACUAAGUGUAGAUGACGGCC |
| | Cytomegalovirus complete | | GUACGGAUUCUAC |
| | genoome | | |
| | Cas12 Variant crRNA targeting | 1389 | UAAUUUCUACUAAGUGUAGAUGCUGACU |
| | Cytomegalovirus complete | | GGAACGUCGUACG |
| | genome | | |
| | Cas12 Variant crRNA targeting | 1390 | UAAUUUCUACUAAGUGUAGAUUGGAUG |
| | Goatpox virus P32 | | GGAUACAUAGUAAG |
| | Cas12 Variant crRNA targeting | 1391 | UAAUUUCUACUAAGUGUAGAUCUCUAUU |
| | Goatpox virus P32 | | CCAUAUACCGUUU |
| | Cas12 Variant crRNA targeting | 1392 | UAAUUUCUACUAAGUGUAGAUAUACGU |
| | Goatpox virus P32 | | AAAUAACAUACCUG |
| | Cas12 Variant crRNA targeting | 1390 | UAAUUUCUACUAAGUGUAGAUUGGAUG |
| | Sheeppox virus P32 | | GGAUACAUAGUAAG |
| | Cas12 Variant crRNA targeting | 1393 | UAAUUUCUACUAAGUGUAGAUUUUUAU |
| | Sheeppox virus P32 | | GGCAGACGAUAAUC |
| | Cas12 Variant crRNA targeting | 1391 | UAAUUUCUACUAAGUGUAGAUCUCUAUU |
| | Sheeppox virus P32 | | CCAUAUACCGUUU |
| | Cas12 Variant crRNA targeting | 1394 | UAAUUUCUACUAAGUGUAGAUAAAAUU |
| | Lumpy Skin Disease Virus P32 | | UUCAACACCUCCUG |
| | Cas12 Variant crRNA targeting | 1395 | UAAUUUCUACUAAGUGUAGAUACAAAG |
| | Lumpy Skin Disease Virus P32 | | AGCAUUACAUAAUC |
| | Cas12 Variant crRNA targeting | 1396 | UAAUUUCUACUAAGUGUAGAUCACCUCC |
| | Lumpy Skin Disease Virus P32 | | UGAUUUUUCUACC |
| | Cas12 Variant crRNA targeting | 1397 | UAAUUUCUACUAAGUGUAGAUUCCUAUG |
| | Mycoplasma capricolum | | GGGUGGGGUUGUA |
| | capripneumoniae 23S | | |
| | Cas12 Variant crRNA targeting | 1398 | UAAUUUCUACUAAGUGUAGAUAUAUGA |
| | Mycoplasma capricolum | | UGGGAACCUAGGGA |
| | capripneumoniae 23S | | |
| | Cas12 Variant crRNA targeting | 1399 | UAAUUUCUACUAAGUGUAGAUGUAUGU |
| | Mycoplasma capricolum | | AGUAGUAGACACGA |
| | capripneumoniae 23S | | |
| | Cas12 Variant crRNA targeting | 1400 | UAAUUUCUACUAAGUGUAGAUGGUUCGC |
| | Mycoplasma capricolum | | CUUGAAGACUGUU |
| | capripneumoniae 16S | | |
| | Cas12 Variant crRNA targeting | 1401 | UAAUUUCUACUAAGUGUAGAUAAGACU |
| | Mycoplasma capricolum | | GUUUUACUAGAAUG |
| | capripneumoniae 16S | | |
| | Cas12 Variant crRNA targeting | 1402 | UAAUUUCUACUAAGUGUAGAUUAAGGG |
| | Mycoplasma capricolum | | AAGAAAAAAUAGAG |
| | capripneumoniae 16S | | |
| | Cas12 Variant crRNA targeting | 1403 | UAAUUUCUACUAAGUGUAGAUAUGCUCU |
| | Mycoplasma capricolum | | ACCGUGAUCUCUA |
| | capripneumoniae L22 | | |
| | Cas12 Variant crRNA targeting | 1404 | UAAUUUCUACUAAGUGUAGAUGAGAGA |
| | Mycoplasma capricolum | | UACGAAUCAUACUU |
| | capripneumoniae L22 | | |
| | Cas12 Variant crRNA targeting | 1405 | UAAUUUCUACUAAGUGUAGAUAGUAGC |
| | Mycoplasma capricolum | | AACUGCAACUGAUA |
| | capripneumoniae L22 | | |
| | Cas12 Variant crRNA targeting | 1406 | UAAUUUCUACUAAGUGUAGAUUAGGAC |
| | Mycoplasma mycoides | | UUUUUAAGAGUUAG |
| | mycoides 23S | | |
| | Cas12 Variant crRNA targeting | 1407 | UAAUUUCUACUAAGUGUAGAUCCUGCGA |
| | Mycoplasma mycoides | | UAAGUUUGGGGGA |
| | mycoides 23S | | |
| | Cas12 Variant crRNA targeting | 1408 | UAAUUUCUACUAAGUGUAGAUUAAUUU |
| | Mycoplasma mycoides | | AUGCACAGCAGUCA |
| | mycoides 23S | | |
| | Cas12 Variant crRNA targeting | 1409 | UAAUUUCUACUAAGUGUAGAUGUUCGU |
| | Mycoplasma mycoides | | GCCGUAAGGUGUUG |
| | mycoides 16S | | |
| | Cas12 Variant crRNA targeting | 1410 | UAAUUUCUACUAAGUGUAGAUGUCGCUA |
| | Mycoplasma mycoides | | GUUACUAACAUUA |
| | mycoides 16S | | |
| | Cas12 Variant crRNA targeting | 1411 | UAAUUUCUACUAAGUGUAGAUCUGUUG |
| | Mycoplasma mycoides | | UAAGGGAAGAAAAA |
| | mycoides 16S | | |
| | Cas12 Variant crRNA targeting | 1412 | UAAUUUCUACUAAGUGUAGAUACGGAA |
| | Mycoplasma mycoides | | UAAAAGAAUGGGGA |
| | mycoides lppB | | |
| | Cas12 Variant crRNA targeting | 1413 | UAAUUUCUACUAAGUGUAGAUCAGUUG |
| | Mycoplasma mycoides | | GAGCUAACCAAAAC |
| | mycoides lppB | | |
| | Cas12 Variant crRNA targeting | 1414 | UAAUUUCUACUAAGUGUAGAUUUUGAU |
| | Mycoplasma mycoides | | CUGCCUUAAAGGUA |
| | mycoides lppB | | |
| | Cas12 Variant crRNA targeting | 1415 | UAAUUUCUACUAAGUGUAGAUUGUAAA |
| | Enterobacter cloacae | | GCCGCCCUCUCGGC |
| | Cas12 Variant crRNA targeting | 1416 | UAAUUUCUACUAAGUGUAGAUUAAGCGC |
| | Enterobacter cloacae | | AGCGCCACCGGGC |
| | Cas12 Variant crRNA targeting | 1417 | UAAUUUCUACUAAGUGUAGAUGGUGAG |
| | Enterobacter cloacae | | GGCAUCAGGCCGCA |
| | Cas12 Variant crRNA targeting | 1418 | UAAUUUCUACUAAGUGUAGAUCAGCGAC |
| | Klebsiella oxytoca | | UCGGAUUCCGACA |
| | Cas12 Variant crRNA targeting | 1419 | UAAUUUCUACUAAGUGUAGAUAGCCCGG |
| | Klebsiella oxytoca | | ACAGAUGCGCAGC |
| | Cas12 Variant crRNA targeting | 1420 | UAAUUUCUACUAAGUGUAGAUCAGUGAC |
| | Klebsiella oxytoca | | UCGGAUUCGGACA |
| | Cas12 Variant crRNA targeting | 1421 | UAAUUUCUACUAAGUGUAGAUGUUACUC |
| | Klebsiella pneumoniae | | UCCGGACUCCGCA |
| | Cas12 Variant crRNA targeting | 1422 | UAAUUUCUACUAAGUGUAGAUCAGCCAC |
| | Klebsiella pneumoniae | | UCUGCCGGUAUGU |
| | Cas12 Variant crRNA targeting | 1423 | UAAUUUCUACUAAGUGUAGAUCCCCGCU |
| | Klebsiella pneumoniae | | GGCGCGGGGAACA |
| | Cas12 Variant crRNA targeting | 1424 | UAAUUUCUACUAAGUGUAGAUCAAGCUA |
| | Cryptococcus neoformans | | UGCAUCCAUGCAU |
| | Cas12 Variant crRNA targeting | 1425 | UAAUUUCUACUAAGUGUAGAUGUUCGCU |
| | Cryptococcus neoformans | | CUAUCGAAGGGUG |
| | Cas12 Variant crRNA targeting | 1426 | UAAUUUCUACUAAGUGUAGAUUACAAG |
| | Cryptococcus neoformans | | UCGCAUUUCGGAGC |
| | Cas12 Variant crRNA targeting | 1427 | UAAUUUCUACUAAGUGUAGAUUCGGUAC |
| | Corynebacterium | | GAGAUGAGCCCGC |
| | pseudotuberculosis chrA | | |
| | Cas12 Variant crRNA targeting | 1428 | UAAUUUCUACUAAGUGUAGAUAUGUGA |
| | Corynebacterium | | GUCGCCGCGACCGA |
| | pseudotuberculosis chrA | | |
| | Cas12 Variant crRNA targeting | 1429 | UAAUUUCUACUAAGUGUAGAUACGACUU |
| | Corynebacterium | | UCGUGCCGUAGCU |
| | pseudotuberculosis chrA | | |
| | Cas12 Variant crRNA targeting | 1310 | UAAUUUCUACUAAGUGUAGAUACGGGG |
| | Escherichia coli 16S | | GCCGCACAAGCGGU |
| | Cas12 Variant crRNA targeting | 1430 | UAAUUUCUACUAAGUGUAGAUAGUCUCG |
| | Escherichia coli 16S | | UAGAGGGGGGUAG |
| | Cas12 Variant crRNA targeting | 1431 | UAAUUUCUACUAAGUGUAGAUGCUGCUU |
| | Escherichia coli 16S | | UGCUGACGAGUGG |
| | Cas12 Variant crRNA targeting | 1432 | UAAUUUCUACUAAGUGUAGAUAGGCUUC |
| | Klebsiella variicola | | GGGUCGGGCCGAG |
| | Cas12 Variant crRNA targeting | 1433 | UAAUUUCUACUAAGUGUAGAUGCUCCGG |
| | Klebsiella variicola | | CCAGCAAAAUCGC |
| | Cas12 Variant crRNA targeting | 1434 | UAAUUUCUACUAAGUGUAGAUGUCGGGC |
| | Klebsiella variicola | | CGAGGCAGCGUCC |
| | Cas12 Variant crRNA targeting | 1435 | UAAUUUCUACUAAGUGUAGAUAACCUUC |
| | Vibrio parahaemolyticus 23S | | GGGGAACGAUAAC |
| | Cas12 Variant crRNA targeting | 1436 | UAAUUUCUACUAAGUGUAGAUCUCGCGU |
| | Vibrio parahaemolyticus 23S | | CAGGAUAUGCCUA |
| | Cas12 Variant crRNA targeting | 1378 | UAAUUUCUACUAAGUGUAGAUAUAGCG |
| | Vibrio parahaemolyticus 23S | | UAUUCGUUUGACGU |

### Reporter

Reporters, which can be referred to interchangeably reporters, or detector nucleic acids, described herein are compatible for use in the devices described herein (e.g., pneumatic valve devices, sliding valve devices, rotating valve devices, and lateral flow devices) and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). Described herein is a reporter comprising a single stranded detector nucleic acid comprising a detection moiety, wherein the reporter is capable of being cleaved by the activated programmable nuclease, thereby generating a first detectable signal. As used herein, a detector nucleic acid is used interchangeably with reporter or reporter. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid may comprise at least 2, 3, 4, 5, 6, 7, 8, 9, or 10 ribonucleotide residues at an internal position. In some cases, the detector nucleic acid may comprise from 2 to 10, from 3 to 9, from 4 to 8, or from 5 to 7 ribonucleotide residues at an internal position. In some cases, the detector nucleic acid may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid may comprise nucleotides resistant to cleavage by the programmable nuclease described herein. In some cases, the detector nucleic acid may comprise synthetic nucleotides. In some cases, the detector nucleic acid may comprise at least one ribonucleotide residue and at least one non-ribonucleotide residue. In some cases, the detector nucleic acid is 5-20, 5-15, 5-10, 7-20, 7-15, or 7-10 nucleotides in length. In some cases, the detector nucleic acid is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the detector nucleic acid may comprise at least one uracil ribonucleotide. In some cases, the detector nucleic acid may comprise at least two uracil ribonucleotides. Sometimes the detector nucleic acid has only uracil ribonucleotides. In some cases, the detector nucleic acid may comprise at least one adenine ribonucleotide. In some cases, the detector nucleic acid may comprise at least two adenine ribonucleotide. In some cases, the detector nucleic acid has only adenine ribonucleotides. In some cases, the detector nucleic acid may comprise at least one cytosine ribonucleotide. In some cases, the detector nucleic acid may comprise at least two cytosine ribonucleotide. In some cases, the detector nucleic acid may comprise at least one guanine ribonucleotide. In some cases, the detector nucleic acid may comprise at least two guanine ribonucleotide. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

### Signals

The devices, systems, fluidic devices, kits, and methods for detecting the presence of a target nucleic acid in a sample described herein may comprise a generation of a signal indicative of the presence or absence of the target nucleic acid in the sample. The generation of a signal indicative of the presence or absence of the target nucleic acid in the sample as described herein is compatible with the methods and devices described herein (e.g., pneumatic valve devices, sliding valve devices, rotating valve devices, and lateral flow devices) and may result from the use of compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). As disclosed herein, in some embodiments, detecting the presence or absence of a target nucleic acid of interest involves measuring a signal emitted from a detection moiety present in a reporter, after cleavage of the reporter by an activated programmable nuclease. Alternatively, or in combination, in some embodiments, detecting the presence or absence of a target nucleic acid of interest involves measuring a signal emitted from a conjugate bound to a detection moiety present in a reporter, after cleavage of the reporter by an activated programmable nuclease. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety or another affinity molecule of the cleaved detector molecule as described herein. Thus, the detecting steps disclosed herein involve indirectly (e.g., via a reporter) measuring the presence of a target nucleic acid, quantifying how much of the target nucleic acid is present, or, measuring a signal indicating that the target nucleic acid is absent in a sample. In some embodiments, a signal is generated upon cleavage of the detector nucleic acid by the programmable nuclease. In other embodiments, the signal changes upon cleavage of the detector nucleic acid by the programmable nuclease. In other embodiments, a signal may be present in the absence of detector nucleic acid cleavage and disappear upon cleavage of the target nucleic acid by the programmable nuclease. For example, a signal may be produced in a microfluidic device or lateral flow device after contacting a sample with a composition comprising a programmable nuclease.

### Buffers

The reagents described herein can also include buffers, which are compatible with the devices, systems, fluidic devices, kits, and methods disclosed herein. The buffers described herein are compatible for use in the devices described herein (e.g., pneumatic valve devices, sliding valve devices, rotating valve devices, and lateral flow devices) and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether the target nucleic acid is in the sample (e.g., DETECTR reactions). These buffers are compatible with the other reagents, samples, and support mediums as described herein for detection of an ailment, such as a disease, cancer, or genetic disorder, or genetic information, such as for phenotyping, genotyping, or determining ancestry. The methods described herein can also include the use of buffers, which are compatible with the methods disclosed herein. For example, a buffer may comprise HEPES, MES, TCEP, EGTA, Tween 20, KCl, MgCl², glycerol, or any combination thereof. In some instances, a buffer may comprise Tris-HCl pH 8.8, VLB, EGTA, CH3COOH, TCEP, IsoAmp®, (NH4)2SO4, KCl, MgSO4, Tween20, KOAc, MgOAc, BSA, TCEP, or any combination thereof. In some instances the buffer may comprise from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer may comprise 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl². The buffer can comprise 0 to 25, 0 to 20, 0 to 10, 0 to 5, 5 to 10, 5 to 15, 5 to 20, 5 to 25, 5 to 30% glycerol. The buffer can comprise from 0% to 30%, from 5% to 30%, from 10% to 30%, from 15% to 30%, from 20% to 30%, from 25% to 30%, from 0% to 25%, from 2% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% glycerol. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Tris-HCl pH 8.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KOAc. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM MgOAc. In some instances the buffer may comprise from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM EGTA. The buffer can comprise from 0% to 30%, from 5% to 30%, from 10% to 30%, from 15% to 30%, from 20% to 30%, from 25% to 30%, from 0% to 25%, from 2% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% Tween 20.

### Compositions Comprising One or More Additives for Improving Assay Signals

In some embodiments, the reagents described herein may include a composition for improving detection signal strength, detection reaction time, detection reaction efficiency, stability, solubility, or the like. In some embodiments, the composition may comprise one or more additives. The one or more additives may, for example, comprise amino acids or derivatives thereof, chaotrpes, chelators, cyclodextrins, inhibitors, ionic liquids, linkers, metals, non detergent sulfobetaines, organic acids, osmolytes, peptides, polyamides, polymers, polyols, polyols and salts, salts, or combinations thereof. In some embodiments, the one or more additives may, for example, comprise one or more of trichloroacetic acid, L-Arginine, L-Glutamic acid, glycine, L-Proline, L-Histidine, beta (B)-Alanine, L-Serine, L-Arginine ethyl ester dihydrochloride, L-Argininamide dihydrochloride, 6-Aminohexanoic acid, Gly-gly peptide, Gly-gly-gly peptide, tryptone, betaine monohydrate, D-(+)-Trehalose dihydrate, Xylitol, D-Sorbitol, sucrose, hydroxyectoine, Trimethylamine N-oxide dihydrate, methyl alpha (α)-D-gluocopyranoside, triethylene glycol, spermine tetrahydrochloride, spermidine, 5-aminovaleric acid, glutaric acid, adipic acid, ethylenediamine dihydrochloride, guanidine hydrochloride, urea, N-methylurea, N-ethylurea, N-methylformamide, hypotauring, TCEP hydrochloride, GSH (L-Glutathione reduced), GSSG (L-Glutathione oxidized), benzaminidine hydrochloride, ethylenediaminetetraacetic acid disodium salt dihydrate, magnesium chloride hexahydrate, calcium chloride dihydrate, cadmium chloride hydrate, cobalt (II) chloride hexahydrate, Non Detergent Sulfobetaine 195 (NDSB-195), NDSB-201, NDSB-211, NDSB-221, NDSB-256, taurine, acetamide, oxalic acid dihydrate, sodium malonate pH 7.0, succinic acid pH 7.0, tacsimate pH 7.0, tetraethylammonium bromide, cholin acetate, 1-Ethyl-3-methylimidazolium acetate, 1-Butyl-3-methylimidazolium chloride, ethylammonium nitrate, ammonium sulfate, ammonium chloride, magnesium sulfate hydrate, potassium thiocynate, gadolinium (III) chloride hexahydrate, cesium chloride, 4-aminobutyric acid (GABA), lithium nitrate, DL-malic acid pH 7.0, lithium citrate tribasic tetrahydrate, ammonium acetate, sodium benzenesulfonate, sodium p-toluenesulfonate, sodium chloride, potassium chloride, sodium phosphate monobasic monohydrate, potassium phosphate dibasic, sodium sulfate decahydrate, lithium chloride, sodium bromide, glycerol, ethylene glycol, polyethylene glycol 200 (PEG-200), PEG 3350, PEG 8000, PEG monomethyl ether 550, PEG monomethyl ether 750, PEG monomethyl ether 1900, formamide, polypropylene glycol P 400, pentaerythritol ethoxylate, 1,2-Propanediol, polyvinylpyrrolidone K 15, 6-O-α-Maltosyl-β-cyclodextrin, (2-Hydroxypropyl)-β-cyclodextrin, α-cyclodextrin, β-cyclodextrin, Methyl-β-cyclodextrin, or any combination thereof.

In some embodiments, the reagents described herein may include a composition for increasing the signal strength of any of the assays described herein. In some embodiments, the composition comprises water and an additive. In some embodiments, the additive may comprise trehalose, xylitol, D-sorbitol, sucrose, and trimethylamine N-oxide dihydrate, or any combination thereof. In some embodiments, the composition further comprises one or more targets, one or more enzymes, one or more reporters, one or more substrates, or any combination thereof as described herein.

In some embodiments, the reagents described herein may include a composition for reducing the time it takes for the signal of any of the assays described herein to saturate. In some embodiments, the composition comprises an additive. In some embodiments, the additive may comprise betaine monohydrate, acetamide, GABA, L-proline, beta-alanine, 6-aminohexanonic acid, urea, methylurea, ethylurea, hypotaurine, NDSB-256, ammonium acetate, or any combination thereof. In some embodiments, the composition further comprises one or more targets, one or more enzymes, one or more reporters, one or more substrates, or any combination thereof as described herein.

In some embodiments, the additive is present at a concentration of at least about 1 nM, at least about 2 nM, at least about 3 nM, at least about 4 nM, at least about 5 nM, at least about 6 nM, at least about 7 nM, at least about 8 nM, at least about 9 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 μM, at least about 2 μM, at least about 3 μM, at least about 4 μM, at least about 5 μM, at least about 6 μM, at least about 7 μM, at least about 8 μM, at least about 9 μM, at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 200 μM, at least about 300 μM, at least about 400 μM, at least about 500 μM, at least about 600 μM, at least about 700 μM, at least about 800 μM, at least about 900 μM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 20 mM, at least about 30 mM, at least about 40 mM, at least about 50 mM, at least about 60 mM, at least about 70 mM, at least about 80 mM, at least about 90 mM, at least about 100 mM, at least about 200 mM, at least about 300 mM, at least about 400 mM, at least about 500 mM, at least about 600 mM, at least about 700 mM, at least about 800 mM, at least about 900 mM, at least about 1 M, at least about 2 M, at least about 3 M, at least about 4 M, at least about 5 M, at least about 6 M, at least about 7 M, at least about 8 M, at least about 9 M, or at least about 10 M.

In some embodiments, the composition increases the signal strength by at least about a factor of 1.01, at least about a factor of 1.02, at least about a factor of 1.03, at least about a factor of 1.04, at least about a factor of 1.05, at least about a factor of 1.06, at least about a factor of 1.07, at least about a factor of 1.08, at least about a factor of 1.09, at least about a factor of 1.1, at least about a factor of 1.2, at least about a factor of 1.3, at least about a factor of 1.4, at least about a factor of 1.5, at least about a factor of 1.6, at least about a factor of 1.7, at least about a factor of 1.8, at least about a factor of 1.9, at least about a factor of 2, at least about a factor of 3, at least about a factor of 4, at least about a factor of 5, at least about a factor of 6, at least about a factor of 7, at least about a factor of 8, at least about a factor of 9, at least about a factor of 10, at least about a factor of 20, at least about a factor of 30, at least about a factor of 40, at least about a factor of 50, at least about a factor of 60, at least about a factor of 70, at least about a factor of 80, at least about a factor of 90, at least about a factor of 100, at least about a factor of 200, at least about a factor of 300, at least about a factor of 400, at least about a factor of 500, at least about a factor of 600, at least about a factor of 700, at least about a factor of 800, at least about a factor of 900, or at least about a factor of 1000.

In some embodiments, the composition reduces the time it takes for the signal to saturate by at least about a factor of 1.01, at least about a factor of 1.02, at least about a factor of 1.03, at least about a factor of 1.04, at least about a factor of 1.05, at least about a factor of 1.06, at least about a factor of 1.07, at least about a factor of 1.08, at least about a factor of 1.09, at least about a factor of 1.1, at least about a factor of 1.2, at least about a factor of 1.3, at least about a factor of 1.4, at least about a factor of 1.5, at least about a factor of 1.6, at least about a factor of 1.7, at least about a factor of 1.8, at least about a factor of 1.9, at least about a factor of 2, at least about a factor of 3, at least about a factor of 4, at least about a factor of 5, at least about a factor of 6, at least about a factor of 7, at least about a factor of 8, at least about a factor of 9, at least about a factor of 10, at least about a factor of 20, at least about a factor of 30, at least about a factor of 40, at least about a factor of 50, at least about a factor of 60, at least about a factor of 70, at least about a factor of 80, at least about a factor of 90, at least about a factor of 100, at least about a factor of 200, at least about a factor of 300, at least about a factor of 400, at least about a factor of 500, at least about a factor of 600, at least about a factor of 700, at least about a factor of 800, at least about a factor of 900, or at least about a factor of 1000. In some embodiments, the additive is present at a concentration of at least about 1 nM, at least about 2 nM, at least about 3 nM, at least about 4 nM, at least about 5 nM, at least about 6 nM, at least about 7 nM, at least about 8 nM, at least about 9 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 μM, at least about 2 μM, at least about 3 μM, at least about 4 μM, at least about 5 μM, at least about 6 μM, at least about 7 μM, at least about 8 μM, at least about 9 μM, at least about 10 μM, at least about 20 μM, at least about 30 μM, at least about 40 μM, at least about 50 μM, at least about 60 M, at least about 70 μM, at least about 80 μM, at least about 90 μM, at least about 100 μM, at least about 200 μM, at least about 300 μM, at least about 400 μM, at least about 500 μM, at least about 600 μM, at least about 700 μM, at least about 800 μM, at least about 900 μM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 20 mM, at least about 30 mM, at least about 40 mM, at least about 50 mM, at least about 60 mM, at least about 70 mM, at least about 80 mM, at least about 90 mM, at least about 100 mM, at least about 200 mM, at least about 300 mM, at least about 400 mM, at least about 500 mM, at least about 600 mM, at least about 700 mM, at least about 800 mM, at least about 900 mM, at least about 1 M, at least about 2 M, at least about 3 M, at least about 4 M, at least about 5 M, at least about 6 M, at least about 7 M, at least about 8 M, at least about 9 M, at least about 10 M.

In some embodiments, the one or more enzymes may comprise a nuclease. In some embodiments, the one or more enzymes may comprise a programmable nuclease. In some embodiments, the one or more enzymes may comprise a Cas12 enzyme. In some embodiments, the one or more enzymes may comprise a Cas14 enzyme. In some embodiments, the one or more enzymes may comprise a CasPhi enzyme. In some embodiments, the one or more enzymes may comprise a Cas13 enzyme. In some embodiments, the one or more enzymes may comprise HRP. In some embodiments, the one or more enzymes may comprise any one or combination of enzymes presented in this disclosure.

In some embodiments, the one or more reporters may comprise a reporter free in solution. In some embodiments, the one or more reporters may comprise a reporter immobilized on a surface of a support. In some embodiments, the one or more reporters may comprise biotin. In some embodiments, the one or more reporters may comprise a fluorescent moiety. In some embodiments, the one or more reporters may comprise a nucleic acid tether. In some embodiments, the one or more reporters may comprise a linker. In some embodiments, the one or more reporters may comprise any one or combination of reporters presented in this disclosure.

In some embodiments, the one or more supports may comprise a glassy substance. In some embodiments, the one or more substrates may comprise a polymeric substance. In some embodiments, the one or more substrates may comprise a hydrogel. In some embodiments, the one or more substrates may comprise any one or combination of substrates presented in this disclosure.

### DETECTR Device Layout

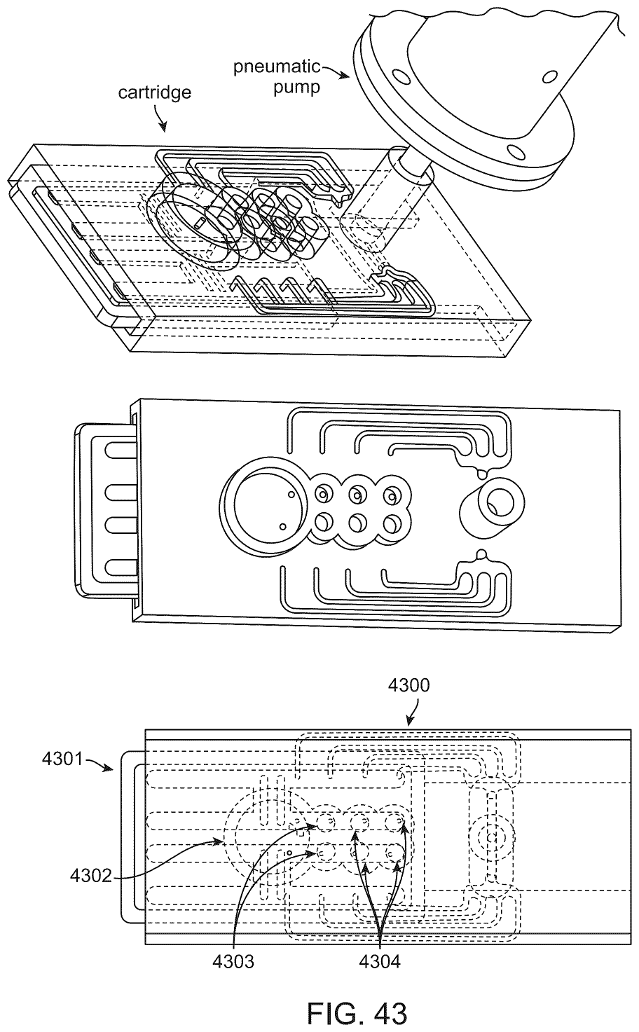

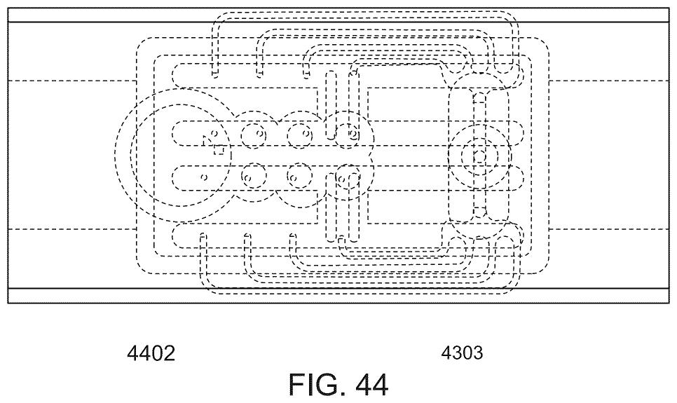

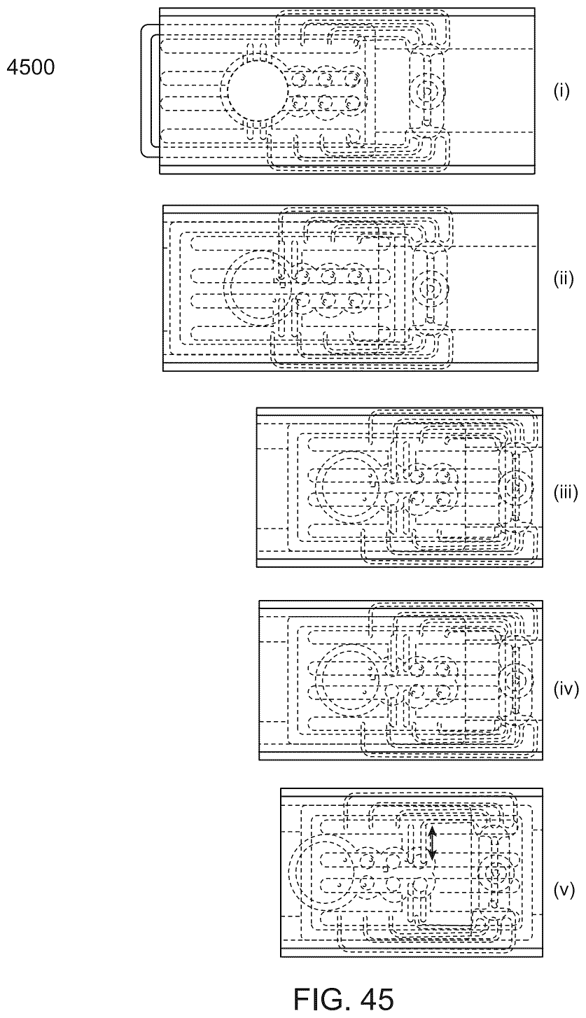

FIG. 43 shows a layout for a device cartridge configured to run a programmable nuclease assay (e.g., a DETECTR assay) as described herein. Shown at top is a pneumatic pump configured to interface with the cartridge. Shown at middle is a top view of the cartridge showing a top layer with reservoirs. Shown at bottom is a sliding valve (4301) containing the sample and arrows pointing to the lysis chamber (4302) at left, followed by amplification chambers (4303) to the right, and detection chambers (4304) further to the right. FIG. 44 shows a schematic of a sliding valve device. The offset pitch of the channels allows aspirating and dispensing into each well separately and helps to mitigate cross talk between the amplification chambers (4402) and corresponding detection chambers (4303). FIG. 45 shows a diagram of sample movement through the sliding valve (4500) device shown in FIG. 44. In the initial closed position (i.), the sample is loaded into the sample well and lysed. The sliding valve (4500) is then actuated by the instrument, and samples are loaded into each of the channels using the pipette pump, which dispenses the appropriate volume into the channel (ii.). The sample is delivered to the amplification chambers by actuating the sliding valve (4500) and mixed with the pipette pump (iii.). Samples from the amplification chamber are aspirated into each channel (iv.) and then dispensed and mixed into each DETECTR chamber (v.) by actuating the sliding valve (4500) and pipette pump. In some embodiments the sliding valve device has a surface area of 5 cm by 8 cm, 5 by 6 cm, 6 by 7 cm, 7 by 8 cm, 8 by 9 cm, 9 by 10 cm, 10 by 11 cm, 11 by 12 cm, 6 by 9 cm, 7 by 10 cm, 8 by 11 cm, 9 by 12 cm, 10 by 13 cm, 11 by 14 cm, 12 by 11 cm, about 30 sq cm, about 35 sq cm, about 40 sq cm, about 45 sq cm, about 50 sq cm, about 55 sq cm, about 60 sq cm, about 65 sq cm, about 70 sq cm, about 75 sq cm, about 25 sq cm, about 20 sq cm, about 15 sq cm, about 10 sq cm, about 5 sq cm, from 1 to 100 sq cm, from 5 to 10 sq cm, from 10 to 15 sq cm, from 15 to 20 sq cm, from 20 to 25 sq cm, from 25 to 30 sq cm, from 30 to 35 sq cm, from 35 to 40 sq cm, from 40 to 45 sq cm, from 45 to 50 sq cm, from 5 to 90 sq cm, from 10 to 0 sq cm, from 15 to 5 sq cm, from 20 to 10 sq cm, or from 25 to 15 sq cm.

In some embodiments, a sliding valve device may comprise a first chamber for sample lysis, a second chamber for detection, and a third chamber for amplification. Another way of referring to these chambers is a sample chamber (e.g., the first chamber), a detection chamber (e.g., the second chamber), and an amplification chamber (e.g., the third chamber). In this layout, the present disclosure provides a device for measuring a signal which may comprise: a sliding layer comprising a channel with an opening at a first end of the channel and an opening at a second end of the channel; and a fixed layer comprising: i) a first chamber having an opening; ii) a second chamber having an opening, wherein the second chamber may comprise a programmable nuclease and a reporter comprising a nucleic acid and a detection moiety; iii) a first side channel having an opening aligned with the opening of the first chamber; and iv) a second side channel having an opening aligned with the opening of the second chamber, wherein the sliding layer and the fixed layer move relative to each other to fluidically connect the first chamber and the first side channel via the opening at the first end of the channel, the opening at the second end of the channel, the opening of the first chamber, and the opening of the first side channel, and wherein the sliding layer and the fixed layer move relative to each other to fluidically connect the second chamber and the second side channel via the opening at the first end of the channel, the opening at the second end of the channel, the opening of the second chamber, and the opening of the second side channel. The fixed layer further may comprise i) a third chamber having an opening; and ii) a third side channel having an opening aligned with the opening of the third chamber, wherein the sliding layer and the fixed layer move relative to each other to fluidically connect the third chamber and the third side channel via the opening at the first end of the channel, the opening at the second end of the channel, the opening of the third chamber, and the opening of the third side channel. The second chamber is coupled to a measurement device for measuring the signal from the detection moiety produced by cleavage of the nucleic acid of the reporter. Additionally, the opening of the first end of the channel overlaps with the opening of the first chamber and the opening of the second end of the channel overlaps with the opening of the first side channel. The opening of the first end of the channel overlaps with the opening of the second chamber and the opening of the second end of the channel overlaps with the opening of the second side channel. The opening of the first end of the channel overlaps with the opening of the third chamber and the opening of the second end of the channel overlaps with the opening of the third channel. Additionally, the first side channel, the second side channel, and the third side channel are fluidically connected to a mixing chamber. In this embodiment, the second chamber additionally includes a guide nucleic acid.

In another embodiment, a sliding valve device may comprise a first chamber for sample lysis and a second chamber for detection. Another way of referring to these chambers is a sample chamber (e.g., the first chamber) and a detection chamber. In this layout, the present disclosure provides a device for measuring a signal may comprise: a sliding layer comprising a channel with an opening at a first end of the channel and an opening at a second end of the channel; and a fixed layer comprising: i) a first chamber having an opening; ii) a second chamber having an opening, wherein the second chamber may comprise a programmable nuclease and a reporter comprising a nucleic acid and a detection moiety; iii) a first side channel having an opening aligned with the opening of the first chamber; and iv) a second side channel having an opening aligned with the opening of the second chamber, wherein the sliding layer and the fixed layer move relative to each other to fluidically connect the first chamber and the first side channel via the opening at the first end of the channel, the opening at the second end of the channel, the opening of the first chamber, and the opening of the first side channel, and wherein the sliding layer and the fixed layer move relative to each other to fluidically connect the second chamber and the second side channel via the opening at the first end of the channel, the opening at the second end of the channel, the opening of the second chamber, and the opening of the second side channel. The second chamber is coupled to a measurement device for measuring the signal from the detection moiety produced by cleavage of the nucleic acid of the reporter. Additionally, the opening of the first end of the channel overlaps with the opening of the first chamber and the opening of the second end of the channel overlaps with the opening of the first side channel. The opening of the first end of the channel overlaps with the opening of the second chamber and the opening of the second end of the channel overlaps with the opening of the second side channel. Additionally, the first side channel and the second side channel are fluidically connected to a mixing chamber. In this embodiment, the second chamber additionally includes a guide nucleic acid.

In some embodiments, the DETECTR assay relies on fluorescence-based detection. In certain embodiments, the DETECTR assay relies on electrochemical-based detection. Electrochemical-based assays have been found to have a lower limit of detection than fluorescence-based assays by roughly two orders of magnitude (Lou et al., 2015).

In some embodiments, electrochemical probes are incorporated into the DETECTR assay to achieve a lower limit of detection. For example, the following electrochemical probe may comprise: 5′-2XXTTATTXX-3′, where 2=5′ 6-FAM; X=ferrocene dT; and 3′=3′ Biotin TEG, where TEG is a 15 atom triethylene glycol spacer. In some embodiments, electrochemical probes are tested with cyclic voltammetry. In some embodiments, electrochemical probes are tested with square wave voltammetry. In some embodiments, a DropSens μSTAT ECL instrument is used for electrochemical measurements. In some embodiments, DropSens screen-printed carbon electrodes are used for electrochemical measurements.

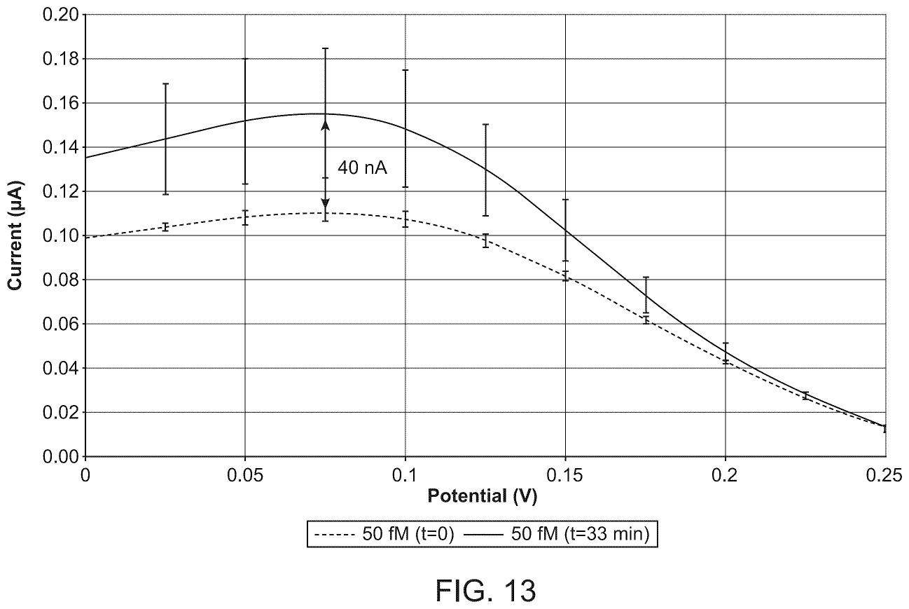

FIG. 13 shows a line graph depicting current as a function of potential. Potential (V) is shown on the x-axis from 0 V to 0.25 V in increments of 0.05 V. Current (μA) is shown on the y-axis from 0 μA to 0.20 μA in increments of 0.02 μA. The graph depicts two lines. The dashed line depicts an oxidation curve for 50 nM HERC2 DETECTR at time=0 of the reaction. The solid line depicts an oxidation curve for 50 nM HERC2 DETECTR at 33 minutes after initiation of the reaction. In this example, error bars represent standard deviation of two measurements of the same solution, using three traces from each measurement. The 40 nA difference in signal indicates detection of the 50 nM HERC2 DETECTR.

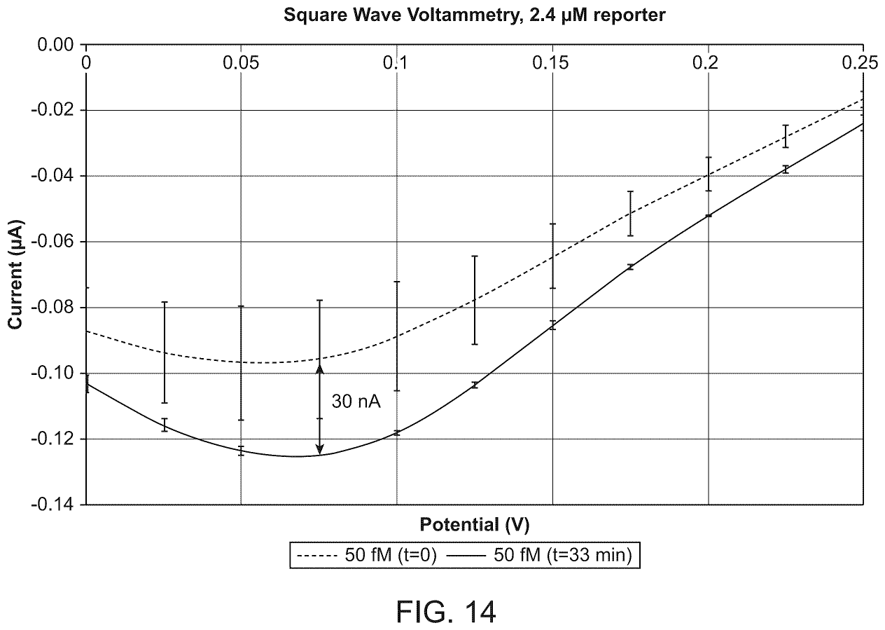

FIG. 14 shows a line graph depicting current as a function of potential. Potential (V) is shown on the x-axis from 0 V to 0.25 V in increments of 0.05 V. Current (μA) is shown on the y-axis from 0 μA to −0.14 μA in increments of 0.02 μA. The graph depicts two lines. The dashed line depicts a reduction curve for HERC2 DETECTR at 50 fM at time=0 for the reaction. The solid line depicts a reduction curve for HERC2 DETECTR at 50 fM at 33 minutes after initiation of the reaction. In this example, error bars represent standard deviation of two measurements of the same solution, using three traces from each measurement.

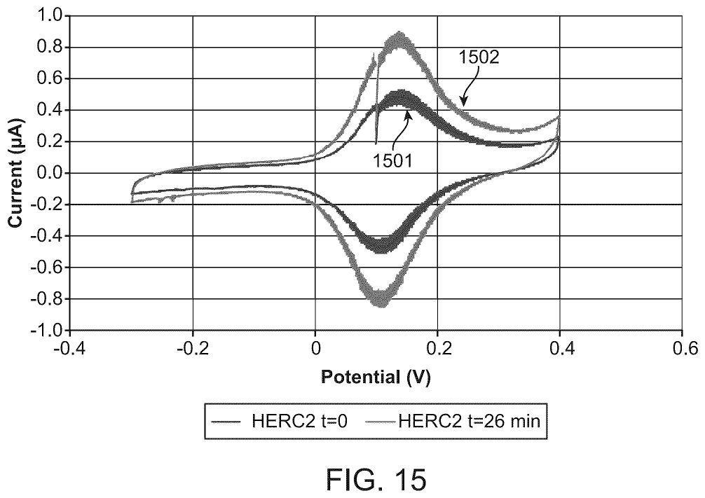

FIG. 15 shows a line graph depicting the current as a function of potential.

Potential (V) is shown on the x-axis from −0.4 V to 0.6 V in increments of 0.2 V. Current (μA) is shown on the y-axis from −1 μA to 1 μA in increments of 0.2 μA. The dark green line (1501) and light green line (1502) depict cyclic voltammograms taken before and after HERC2 DETECTR reaction using 24 μM electrochemical reporter, respectively. In this example, each trace is the average of three scans of the same solution and error bars represent standard deviations. The difference in current between the voltammograms indicates detection of HERC2 DETECTR at 24 μM.

### SARS-CoV-2 Electrochemical DETECTR Reaction

In certain embodiments, described herein DETECTR has been demonstrated to be a powerful technology for detection of pathogens such as SARS-CoV-2 (Broughton et al., 2020). In some embodiments, electrochemical probes are utilized in the DETECTR assay for detection of pathogens. In some embodiments, the electrochemical probe-based DETECTR assay is configured for detection of the pathogen SARS-CoV-2. In some embodiments, the electrochemical probe is 5′-2XXTTATTXX-3′, where 2=5′ 6-FAM; X=ferrocene dT; and 3=3′ BiotinTEG.

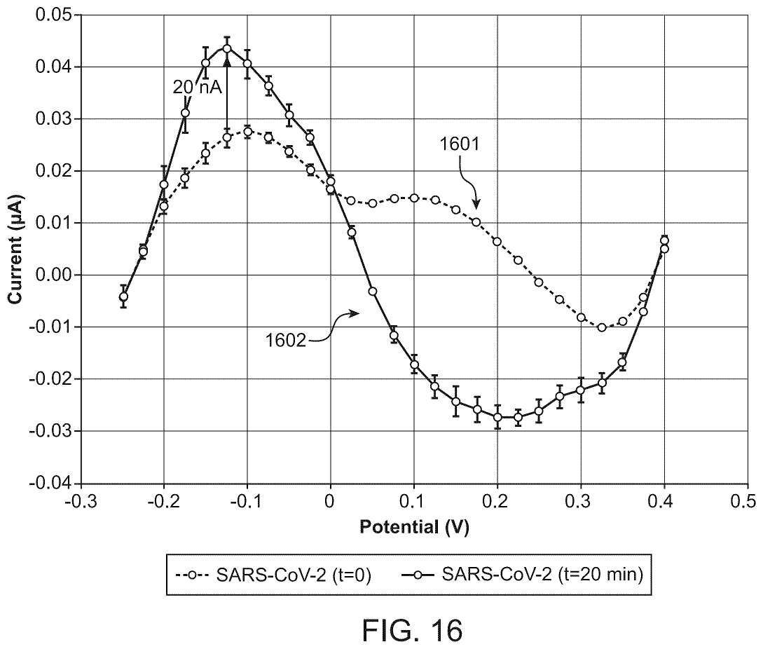

FIG. 16 shows an example line graph depicting current as a function of potential. Potential (V) is shown on the x-axis from −0.3 V to 0.5 V in increments of 0.1 V. Current (A) is shown on the y-axis from −0.04 μA to 0.05 μA in increments of 0.01 μA. The green line (1601) depicts an oxidation curve for SARS-CoV2 at 0 seconds into the reaction and the yellow line (1602) depicts an oxidation curve for SARS-CoV2 at 20 minutes after initiation of the reaction. In some embodiments, error bars represent standard deviation of three traces from each measurement. The oxidation curve at 20 minutes is observed to be 20 nA higher than the oxidation curve at time 0. The 20 nA difference indicates the presence of SARS-CoV2.

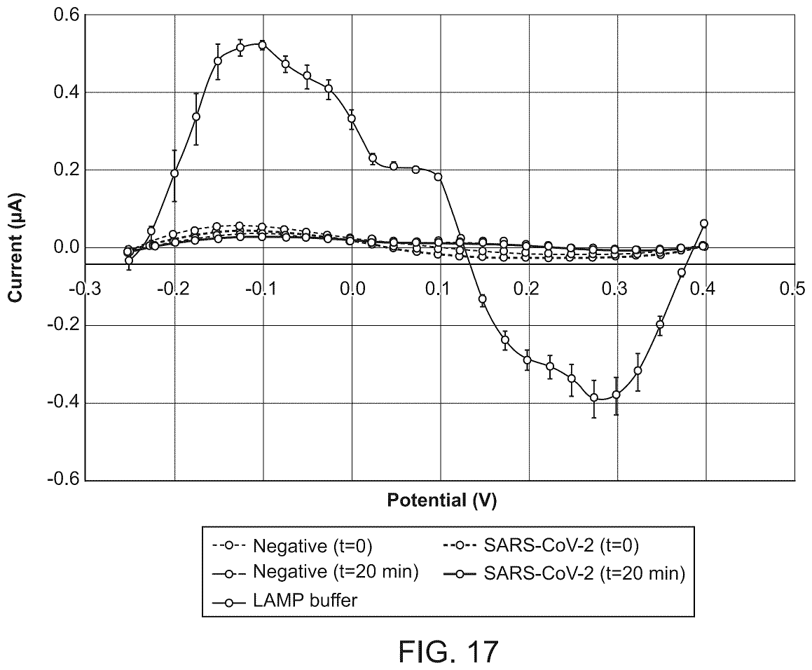

FIG. 17 shows an example line graph depicting current as a function of potential. Potential (V) is shown on the x-axis from −0.3 V to 0.5 V in increments of 0.1 V. Current (μA) is shown on the y-axis from −0.6 μA to 0.6 μA in increments of 0.2 μA. The lines depict square wave voltammetry measurements for SARS-CoV-2 DETECTR reaction with electrochemical reporters and controls.

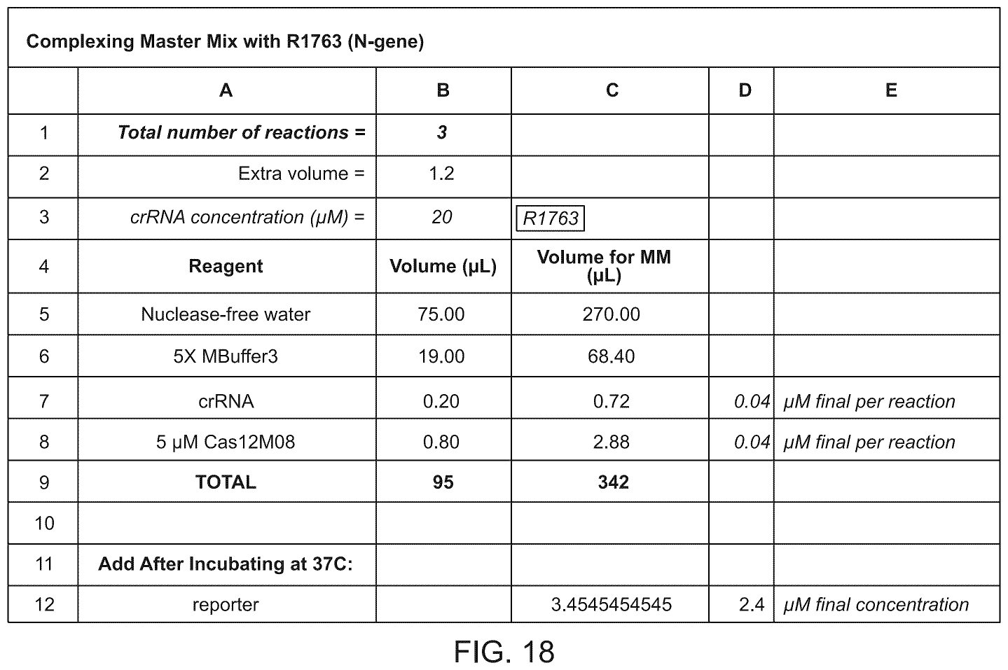

FIG. 18 shows an example of a complexing master mix with R1763 (N-gene).

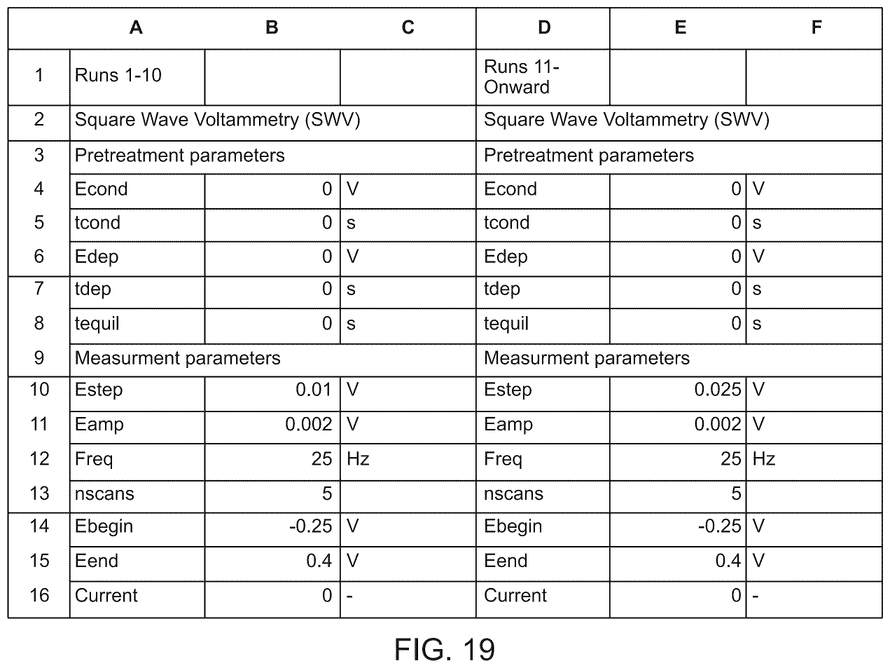

FIG. 19 presents an example of experimental conditions described herein, for square wave voltammetry.

### DETECTR Assay Immobilization

CRISPR diagnostic reactions are generally performed in solution where the Cas protein-RNA complexes can freely bind target nucleic acids and reporters. However, reactions where all components are in solution limit the designs of CRISPR diagnostic assays, especially in microfluidic devices. A system where various components of the CRISPR diagnostic reaction are immobilized on a surface enables designs where multiple readouts can be accomplished within a single reaction chamber.

Described herein are various methods to immobilize CRISPR diagnostic reaction components to the surface of a reaction chamber or other surface (e.g., a surface of a bead). Any of the devices described herein may comprise one or more immobilized detection reagent components (e.g., programmable nuclease, guide nucleic acid, and/or reporter), or component capable of being immobilized at some point during a particular assay (e.g., through the inclusion of a functionality for immobilization to a surface, such as the surface of a bead). In some embodiments, the surface is a bead, which may be present at the beginning of a reaction, or added later to capture one or more components of a reaction (e.g., unreacted reporters comprising an enzyme, a linker, and a functionality for immobilization). The term “bead” is not limited to any particular size or shape. Beads may be uniform or non-uniform, spherical or non-spherical, regular or irregular. In embodiments, beads are magnetic or paramagnetic. The surface of the beads may comprise a reaction partner for the functional moiety for immobilization (e.g., streptavidin coated beads for capture of targets comprising a biotin functionality). In some embodiments, the beads are microparticles or nanoparticles. A variety of beads for use with nucleic acid based assays are commercially available, a non-limiting example of which includes DYNABEADS in various formats. In certain instances, methods include immobilization of programmable nucleases (e.g., Cas proteins or Cas enzymes), reporters, guide nucleic acids (e.g., gRNAs), or a combination of two or all of these. In some embodiments, various CRISPR diagnostic reaction components are modified with biotin. In some embodiments, these biotinylated CRISPR diagnostic reaction components are tested for immobilization on surfaces coated with streptavidin. In some embodiments, the biotin-streptavidin interaction is used as a model system for other immobilization chemistries.

Table 3 presents gRNA and reporter immobilization sequences

TABLE 3
gRNA and reporter immobilization sequences

| | SEQ | | |
|---|---|---|---|
| | ID | | |
| Name | NO: | Sequence | Description |
| R003 | 1437 | rGrGrCrCrArCrCrCrCrArArArArArUrGrArAr | unmodified Cas13 crRNA |
| | | GrGrGrGrArCrUrArArArArCrArGrUrGrArUr | |
| | | ArArGrUrGrGrArArUrGrCrCrArUrG | |
| mod023 | 1438 | /5BiotinTEG/rGrGrCrCrArCrCrCrCrArArArAr | biotin modified Cas13 crRNA |
| | | ArUrGrArArGrGrGrGrArCrUrArArArArCrAr | |
| | | CrGrArCrCrUrArCrUrCrUrCrCrCrArUrArCrU | |
| | | rC | |
| R1763 | 1439 | rUrArArUrUrUrCrUrArCrUrArArGrUrGrUrAr | Unmodified gRNA targeting a |
| | | GrArUrCrCrCrCrCrArGrCrGrCrUrUrCrArGrC | sequence in SARS-CoV-2 |
| | | rGrUrUrC | |
| mod011 | 1440 | /5Biosg/rUrA rArUrU rUrCrU rArCrUrArArG | R1763 with a 5′ biotin |
| | | rUrGrU rArGrArUrCrCrCrCrCrArGrCrGrCrU | |
| | | rUrCrA rGrCrG rUrUrC | |
| mod012 | 1441 | /5Biosg/T*T*T*T*T*rUrArArUrUrUrCrUrAr | R1763 with 5 |
| | | CrUrArArGrUrGrUrArGrArUrCrCrCrCrCrAr | phosphorothioated nucleotides |
| | | GrCrGrCrUrUrCrArGrCrGrUrUrC | on 5′ end |
| mod013 | 1442 | /5Biosg/T*T*T*T*T*T*T*T*T*T*rUrArArUr | R1763 with 10 |
| | | UrUrCrUrArCrUrArArGrUrGrUrArGrArUrCr | phosphorothioated nucleotides |
| | | CrCrCrCrArGrCrGrCrUrUrCrArGrCrGrUrUrC | on 5′ end |
| mod014 | 1443 | rUrArArUrUrUrCrUrA/iBiodUK/rCrUrArArGr | R1763 with an internal biotin |
| | | UrGrUrArGrArUrCrCrCrCrCrArGrCrGrCrUr | modification |
| | | UrCrArGrCrGrUrUrC | |
| mod015 | 1444 | rUrArArUrUrUrCrUrArCrUrArArGrUrGrUrAr | R1763 with an internal biotin |
| | | GrArUrC/iBiodUK/rCrCrCrCrArGrCrGrCrUr | modification |
| | | UrCrArGrCrGrUrUrC | |
| mod016 | 1445 | rUrArArUrUrUrC/iBiodUK/rArCrUrArArGrUr | R1763 with an internal biotin |
| | | GrUrArGrArUrCrCrCrCrCrArGrCrGrCrUrUr | modification |
| | | CrArGrCrGrUrUrC | |
| mod017 | 1446 | rUrArArUrUrUrCrUrArCrUrArArGrUrGrUrAr | R1763 with an internal biotin |
| | | GrA/iBiodUK/rCrCrCrCrCrArGrCrGrCrUrUr | modification |
| | | CrArGrCrGrUrUrC | |
| mod018 | 1447 | /5BiotinTEG/rUrArArUrUrUrCrUrArCrUrArA | R1763 with a 5′ biotin-TEG |
| | | rGrUrGrUrArGrArUrCrCrCrCrCrArGrCrGrCr | modification |
| | | UrUrCrArGrCrGrUrUrC | |
| mod019 | 1448 | rUrA rArUrU rUrCrU rArCrU rArArGrUrGrU | R1763 with a 3′ biotin |
| | | rArGrA rUrCrC rCrCrC rArGrCrGrCrU | modification |
| | | rUrCrA rGrCrG rUrUrC/3Bio/ | |
| mod020 | 1449 | rUrA rArUrU rUrCrU rArCrU rArArGrUrGrU | R1763 with a 3′ biotin-TEG |
| | | rArGrA rUrCrC rCrCrCrArGrCrGrCrU | modification |
| | | rUrCrA rGrCrGrUrUrC/3BioTEG/ | |
| rep072 | | /56-FAM/TTATTATT/3Bio/ | FAM reporter with 3′ biotin |
| rep103 | | /5Alex488N/TT ATT ATT/3Bio/ | 5′ modified with Alexa488 |
| | | | reporter and 3′ modified with |
| | | | biotin |
| rep104 | 1452 | /5Alex488N/TT ATT ATT | 5′ modified with Alexa488 |
| | | AT/iBiodT/ATT/3IABKFQ/ | reporter with internal biotin |
| | | | and 3′ modified with a |
| | | | quencher |
| rep105 | 1453 | /5BiotinTEG/*T*T*/i6-FAMK/*TATTA TTA | 5′ modified with biotin-TEG |
| | | TTA TTA TT/3IABKFQ/ | followed by two |
| | | | phosphorothioated nucleotides |
| | | | (*) should not be cleavable and |
| | | | an internal FAM and 3′ |
| | | | modified with a quencher |
| | | | *T* could also be some other |
| | | | non-cleavable su |
| rep106 | 1454 | /56-FAM/TT ATT ATT ATT A/3Dig_N/ | 5′ FAM reporter with a 3′ DIG |
| rep115 | 1455 | [biotinTEG]TTTTTTTTTTTTTTTTTTTTTTT | biotin TEG modified 5′ and |
| | | TTTTTTT[Phycoerythrin] | Phycoetrythrin modified 3′, |
| | | | where 5′ and 3′ linked by T30 |
| | | | (SEQ ID NO: 1546) |
| rep116 | 1456 | [FAM]n[branch]TTTTTTTTTTTTTTTTTTTT | 5′ modified with 9 FAMs and |
| | | [biotin TEG] | 3′ modified with biotinTEG, |
| | | | where 5′ and 3′ linked by T20 |
| | | | (SEQ ID NO: 126) |
| rep117 | 1457 | [BiotinTEG]*T*T*[internalFAM]*TTTTTTTT | 5′ modified with BiotinTEG |
| | | TTTTTTTTTTTT[IABKFQ] | and 3′ modified with FQ, |
| | | | where 5′ and 3′ linked by T20 |
| | | | (SEQ ID NO: 126) |
| rep118 | 1458 | [FAM]TTTTTTTTTTTTTTTTTTTT* | 5′ modified with FAM and 3′ |
| | | [internalBiotin]*T[IABKFQ] | modified with Biotin-FQ, |
| | | | where 5′ and 3′ are linked by |
| | | | T20 (SEQ ID NO: 126) |
| rep119 | 1459 | [5BiotinTEG]*T*T*[internalCy5]*TTTTTTTT | 5′ modified with BiotinTEG |
| | | TTTTTTTTTTTT[RQ] | and 3′ modified with BHQ-2, |
| | | | contains internal Cy5 dye |
| | | | linked to quencher by T20 |
| | | | (SEQ ID NO: 126) |
| rep121 | 1460 | /5BiotinTEG//iSp18//iCy5/TTT TTT TTT TTT | 5′ modified with BiotinTEG, |
| | | TTTTTT TT/3IAbRQSp/ | an internal 18 atom spacer, and |
| | | | an internal Cy5 followed by |
| | | | T20 (SEQ ID NO: 126) and a |
| | | | IAbRQSp quencher |
| rep120 | 1461 | [Cy5]TTTTTTTTTTTTTTTTTTTT* | 5′ modified with Cy5 followed |
| | | [internalBiotin]*T[RQ] | by T20 linker (SEQ ID NO: |
| | | | 126) to an internal biotin and |
| | | | an RQ quencher. * indicates |
| | | | phosphorothioated nucleotides |
| rep125 | 1462 | /5Alex647N//iBiodT/TTT TTT TTT TTT TTT | 5′ modified with Alexa647 |
| | | TTT TT/3IAbRQSp/ | followed by an internal |
| | | | biotindT T20 (SEQ ID NO: |
| | | | 126) and a 3′ IAbRQSp |
| | | | quencher |
| rep126 | 1463 | /5BiotinTEG/TT/i6- | 5′ biotinTEG modified |
| | | FAMK/TTTTTTTTTrUrUrUrUrUTTTTTT/ | followed by two T, an internal |
| | | 3IABKFQ/ | FAM and, 10T, 5 RNA U, 6T, |
| | | | and a quencher. Acts as an |
| | | | RNA cleavage reporter. |
| rep110 | 1464 | /5AmMC6T/TTTTTTTTTTTT/3AlexF488N/ | 5′ amino with 6 carbons |
| | | | followed by T12 (SEQ ID NO: |
| | | | 127) and a Alexa488 |
| | | | modification on 3′ end |
| rep111 | 1465 | /5AmMC6T//i6- | 5′ amino with 6 carbons |
| | | FAMK/TTTTTTTTTTTT/3IABKFQ/ | followed by internal FAM, |
| | | | T12 (SEQ ID NO: 127), and a |
| | | | quencher on 3′ |
| rep112 | 1466 | /5AmMC6T/TT TTT TTT TTT T/36-FAM/ | 5′ amino with 6 carbons |
| | | | followed by T12 (SEQ ID NO: |
| | | | 127) and a 3′ FAM |
| | | | modification |
| rep122 | 1467 | /5AmMC12//iSp18//iCy5/TTT TTT TTT TTT | 5′ amino with 12 carbons |
| | | TTTTTT TT/3IAbRQSp/ | followed by 18 atom linker |
| | | | followed by Cy5 followed by |
| | | | T20 (SEQ ID NO: 126) and a |
| | | | 3′ quencher |
| rep123 | 1468 | /5AmMC12//iCy5/TTT TTT TTT TTT TTT | 5′ amino with 12 carbons |
| | | TTT TT/3IAbRQSp/ | followed by internal Cy5 and a |
| | | | T20 linker (SEQ ID NO: 126) |
| | | | to a 3′ quencher |
| rep135 | 1469 | /5AmMC12//i6- | 5′ amino with 12 carbons |
| | | FAMK/TTTTTTTTTTTTTTTTTTT/3IABKFQ/ | followed by internal FAM and |
| | | | a T20 linker (SEQ ID NO: |
| | | | 126) to a 3′ quencher |
| rep136 | 1470 | /5AmMC6//i6- | 5′ amino with 6 carbons |
| | | FAMK/TTTTTTTTTTTTTTTTTTT/3IABKFQ | followed by internal FAM and |
| | | | a T20 linker (SEQ ID NO: |
| | | | 126) to a 3′ quencher |
| mod026 | 1471 | /5AmMC6/rUrArArUrUrUrCrUrArCrUrArAr | 5′ amino with 6 carbons linked |
| | | GrUrGrUrArGrArUrCrCrCrCrCrArGrCrGrCr | to gRNA for SARS-CoV-2 N- |
| | | UrUrCrArGrCrGrUrUrC | gene |
| mod027 | 1472 | /5AmMC12/rUrArArUrUrUrCrUrArCrUrArAr | 5′ amino with 12 carbons |
| | | GrUrGrUrArGrArUrCrCrCrCrCrArGrCrGrCr | linked to gRNA for SARS- |
| | | UrUrCrArGrCrGrUrUrC | CoV-2 N-gene |
| mod028 | 1473 | /5AmMC6T/rUrArArUrUrUrCrUrArCrUrArAr | 5′ amino with 6 carbons on dT |
| | | GrUrGrUrArGrArUrCrCrCrCrCrArGrCrGrCr | linked to gRNA for SARS- |
| | | UrUrCrArGrCrGrUrUrC | CoV-2 N-gene |
| mod029 | 1474 | rUrArArUrUrUrCrUrArCrUrArArGrUrGrUrAr | gRNA for SARS-CoV-2 N- |
| | | GrArUrCrCrCrCrCrArGrCrGrCrUrUrCrArGrC | gene with 5′ amino with 6 |
| | | rGrUrUrC/3AmMC6T/ | carbons on 3′ end |
| mod030 | 1475 | /5AmMC12/rUrArArUrUrUrCrUrArCrUrArAr | 5′ amino with 12 carbons |
| | | GrUrGrUrArGrArUrUrUrArCrArUrGrGrCrUr | linked to gRNA for human |
| | | CrUrGrGrUrCrCrGrArG | RNase P POP7 |
| mod031 | 1476 | /5AmMC12/rUrArArUrUrUrCrUrArCrUrArAr | 5′ amino with 12 carbons |
| | | GrUrGrUrArGrArUrGrCrCrGrArUrArArUrGr | linked to gRNA for |
| | | ArUrGrUrArGrGrGrArU | Mammuthus primigenius |
| | | | control sequence |
| mod059 | 1477 | /5ThioMC6-D/rUrA rArUrU rUrCrU rArCrU | 5′ thiol modification with 6 |
| | | rArArG rUrGrU rArGrA rUrCrC rCrCrC | carbons linked to gRNA for |
| | | rArGrC rGrCrU rUrCrA rGrCrG rUrUrC | SARS-CoV-2 N-gene |
| rep130 | 1478 | /5ThioMC6-D/*T*T*/i6- | 5′ modified with BiotinTEG |
| | | FAMK/*TTTTTTTTTTTTTTTTTTTT/3IABkFQ/ | and 3′ modified with FQ, |
| | | | where 5′ and 3′ linked by T20 |
| | | | (SEQ ID NO: 126); two |
| | | | phosphorothioated nucleotides |
| | | | between thiol and internal |
| | | | FAM |
| mod024 | 1479 | /5BiotinTEG/rUrArArUrUrUrCrUrArCrUrArA | 5′ modified with BiotinTEG |
| | | rGrUrGrUrArGrArUrGrCrCrGrArUrArArUrGr | linked to a gRNA that targets |
| | | ArUrGrUrArGrGrGrArU | Mammuthus primigenius |
| | | | sequence |
| mod025 | 1480 | /5BiotinTEG/rUrArArUrUrUrCrUrArCrUrArA | 5′ modified with BiotinTEG |
| | | rGrUrGrUrArGrArUrUrUrArCrArUrGrGrCrUr | linked to a gRNA that targets |
| | | CrUrGrGrUrCrCrGrArG | human RNase P POP7 |
| mod058 | 1481 | /5biotinTEG/rUrArArUrUrUrCrUrArCrUrArA | 5′ modified with BiotinTEG |
| | | rGrUrGrUrArGrArUrCrUrGrCrCrArArUrUrGr | linked to a gRNA that targets |
| | | CrArGrGrArArUrGrArU | Mammuthus primigenius |
| | | | sequence |
| Acrydite- | 1482 | (/5Acryd/TTT TTT TTT TTT TTT TTT TT/i6- | 5′ modified with acrydite for |
| modified | | FAMK//3Bio/). | incorporation into hydrogel |
| Reporter | | | |
| 172 | | | |
| R1763 | 1483 | /5Acryd/UAA UUU CUA CUA AGU GUA | R1763 modified at 5′ end with |
| with | | GAU CCC CCA GCG CUU CAG CGU UC | acrydite for incorporation into |
| Acrydite | | | hydrogel |
| Modifi- | | | |
| cation | | | |
| R1965 | 1484 | /5Acryd/rUrArAr UrUrUr CrUrAr CrUrAr | R1965 modified at 5′ end with |
| with | | ArGrUr GrUrAr GrArUr UrUrAr CrArUr | acrydite for incorporation into |
| Acrydite | | GrGrCr UrCrUr GrGrUr CrCrGr Ar G | hydrogel |
| Modifi- | | | |
| cation | | | |
| rep138 | | /5AmMC12//i6- | 5′ modified with amino |
| | | FAMK/rUrUrUrUrUrUrUrUrU/3IABKFQ/ | modifier C12 followed by an |
| | | | internal 6-FAMK and 3′ |
| | | | IABKFQ quencher |
| rep198 | | /5BiotinTEG//i6- | 5′ modified with BiotinTEG |
| | | FAMK/rUrUrUrUrUrUrUrUrU/3IABkFQ/ | followed by an internal 6- |
| | | | FAMK and 3′ IABKFQ |
| | | | quencher |

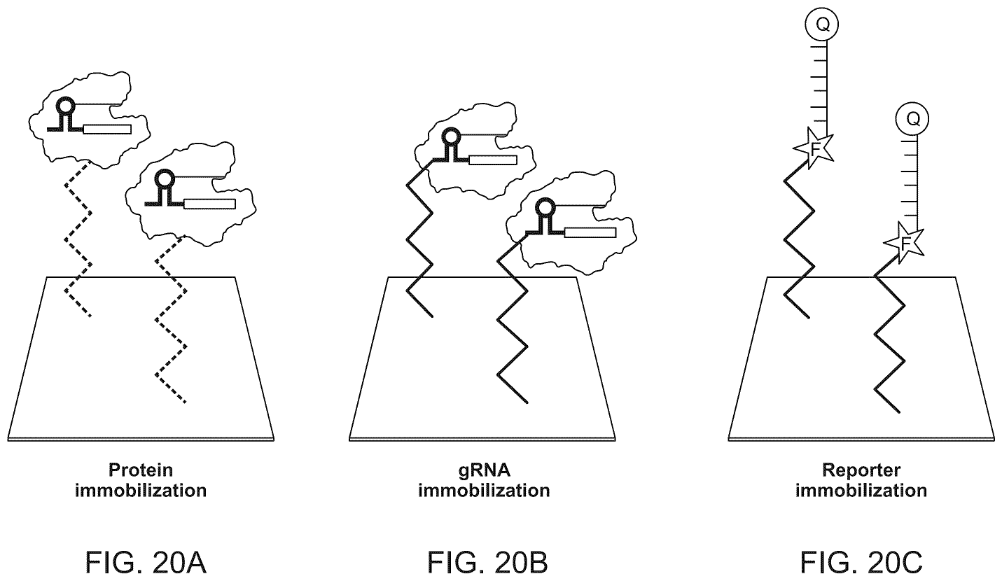

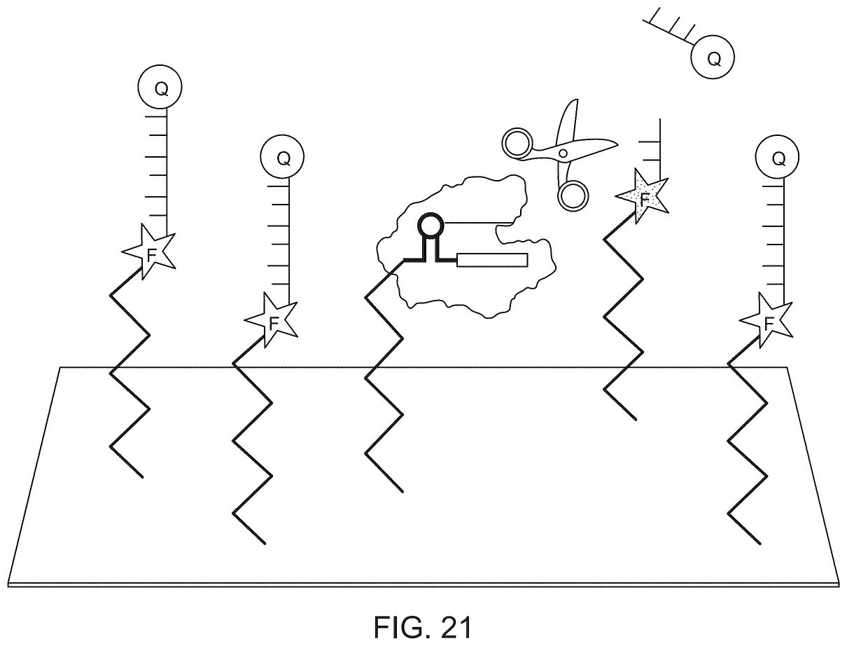

FIGS. 20A-20C illustrate three exemplary immobilization strategies for CRISPR-Cas diagnostic assay components. In some embodiments, as seen in FIG. 20A, chemical modifications of amino acid residues in the Cas protein enable attachment to a surface. In some embodiments, as seen in FIG. 20B, gRNAs are immobilized by adding various chemical modifications at the 5′ or 3′ end of the gRNA that are compatible with a selected surface chemistry. In some embodiments, as seen in FIG. 20C fluorescence-quenching (FQ), or other reporter chemistries, are attached to surfaces using similar chemical modifications as gRNAs. In some embodiments, these attached reporters are activated by a Cas protein, which leads to either activated molecules that remain attached to the surface or activated molecules that are released into solution.

For some embodiments, described herein, FIG. 21 provides an illustrative example of immobilization strategies for use with methods and compositions described herein where the RNP complex is immobilized by a gRNA and cleaves surrounding FQ reporters that are also immobilized to a surface. Here, the quencher is released into solution, leaving a localized fluorescent signal.

In some embodiments, the programmable nuclease, guide nucleic acid, or the reporter are immobilized to a device surface by a linkage or linker. In some embodiments, the linkage comprises a covalent bond, a non-covalent bond, an electrostatic bond, a bond between streptavidin and biotin, an amide bond, or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface.

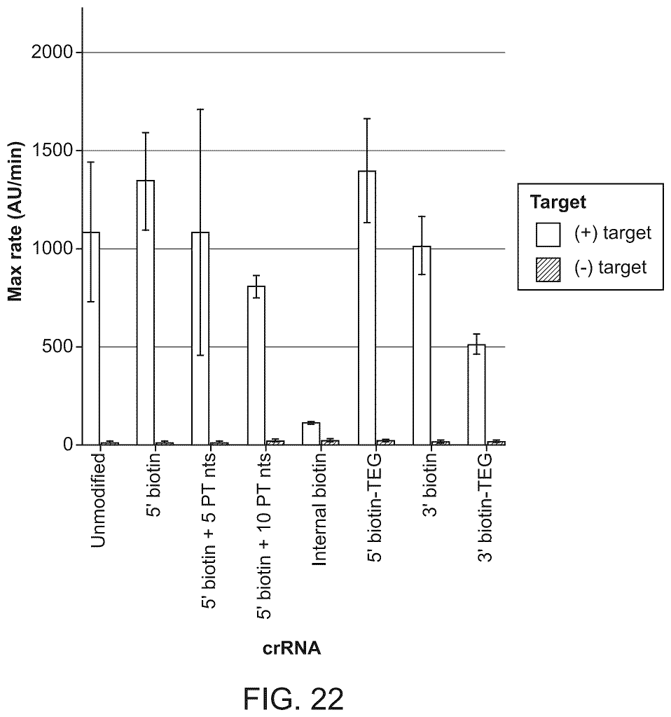

In some embodiments, various chemical modifications to gRNAs are described as shown in FIG. 22. The y-axis shows reaction rate in terms of fluorescence intensity over time and the x-axis presents various modifications of crRNA. In some embodiments, unmodified biotin and variations of biotin modifications are placed at various positions along a Cas12 variant (e.g., SEQ ID NO: 17) gRNA. The modified gRNAs are then complexed with the protein and dsDNA target is added. In some embodiments, higher average fluorescence over the same period of time indicates that modifications are tolerated on the 5′ and 3′ ends of the gRNA, but not internally in the gRNA. 5′ modified gRNAs appear to be more robust than 3′ modifications.

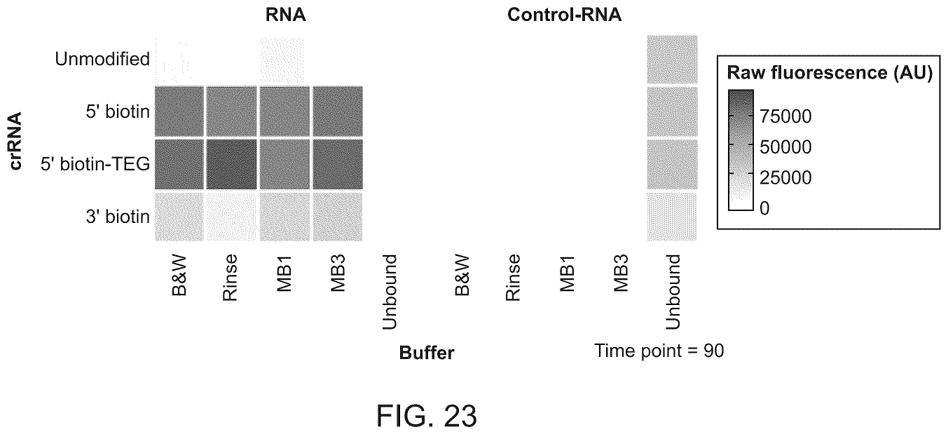

In some embodiments, the immobilization of gRNAs to a streptavidin surface are described as shown in FIG. 23. Two plots are shown, where the left-hand plot depicts RNA bound to a streptavidin coated surface and the right-hand plot depicts unbound control-RNA, in solution, mixed with target, reporter and protein solution. In each table, the y-axis depicts, in some embodiments, various modifications of crRNA and the x-axis depicts various buffer conditions that the crRNA is subjected to. The left-hand, experimental plot shows gRNA on 5′ or 3′ side are both functional approaches, but 5′ biotin modified gRNAs show increased signal in comparison to 3′ modified gRNAs. In some embodiments, unmodified gRNAs show no signal when bound to the plate. Conversely, the right-hand control plot depicts an unbound control where, in some embodiments, free gRNA is mixed with target, reporter, and protein solution. In this embodiment, sufficient signal is observed, indicating functionality in unmodified gRNAs.

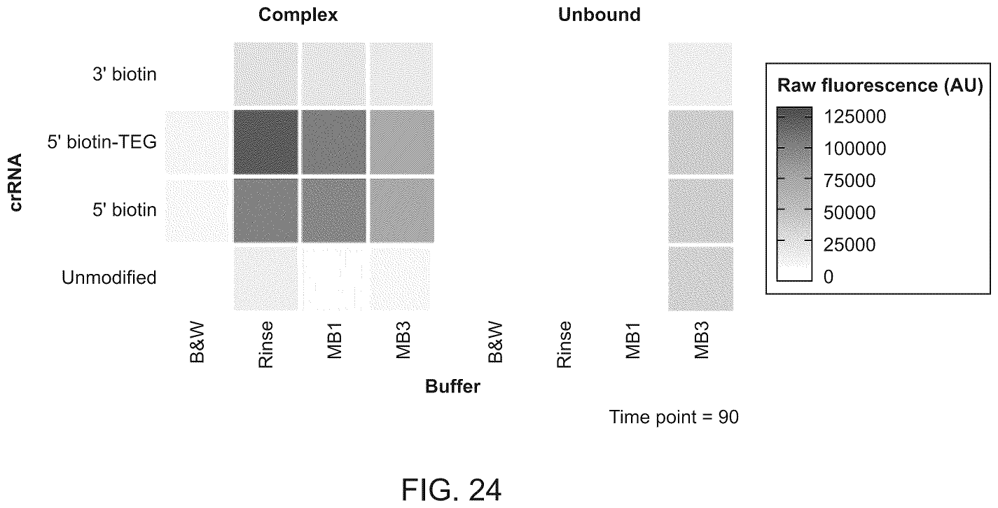

In some embodiments, Cas proteins are complexed with RNA complexes as described herein and shown in FIG. 5. FIG. 24 shows RNP complexes bound by 5′ biotin modified gRNAs exhibit higher signal, indicating functional attachment to the surface of the streptavidin coated plate. Samples exposed to unbound, high salt “B&W” buffer conditions show less fluorescence signal indicating inhibited protein activity or disruption of binding of functional RNP complexes to the surface of the plate. Unmodified gRNAs also exhibit lower fluorescence indicating failed binding to the RNP to the plate surface. Control assays, shown in the right-hand plot, indicate unbound gRNAs are still functional.

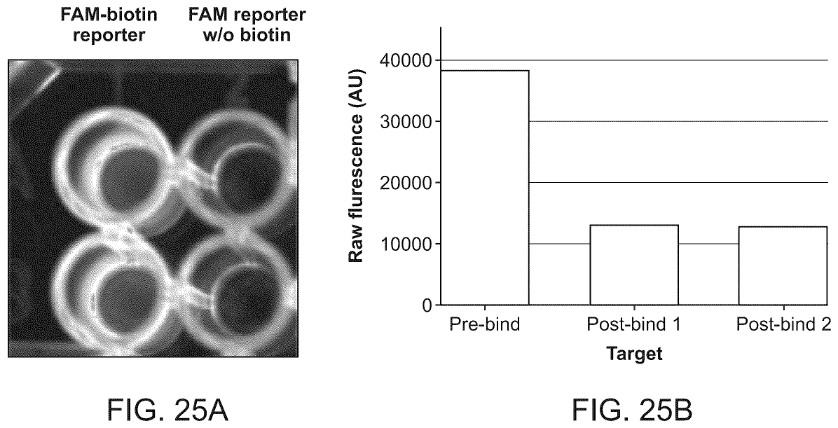

In some embodiments, reporters are immobilized to the surface as shown in FIGS. 25A-25B. FIG. 25A shows a fluorescence image of four wells with streptavidin coated surfaces, where the left-hand column of wells contains FAM-biotin reporters immobilized to a streptavidin coated surface. The right-hand column of wells contains FAM reporter without biotin functionalization. The left-hand column exhibits a higher signal. FIG. 25B shows a comparison of fluorescence intensity of the FAM-biotin pre-binding solution to the solution after incubating on the streptavidin plate. A decrease in signal for both wells containing FAM-biotin is observed.

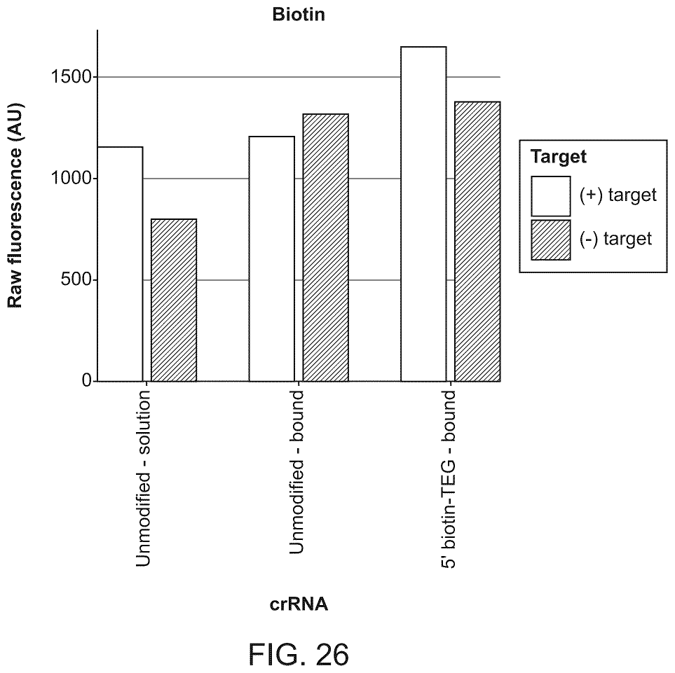

In some embodiments, combined RNP and a reporter system are immobilized for functional testing as shown in FIG. 26. Raw fluorescence (AU) is plotted against three conditions: (1) unmodified crRNA in solution, (2) unmodified crRNA bound to the surface and (3) 5′ biotin-TEG modified crRNA bound to the surface. The combined binding of the reporter and RNP to the plate shows a similar signal to RNP in solution with bound reporter.

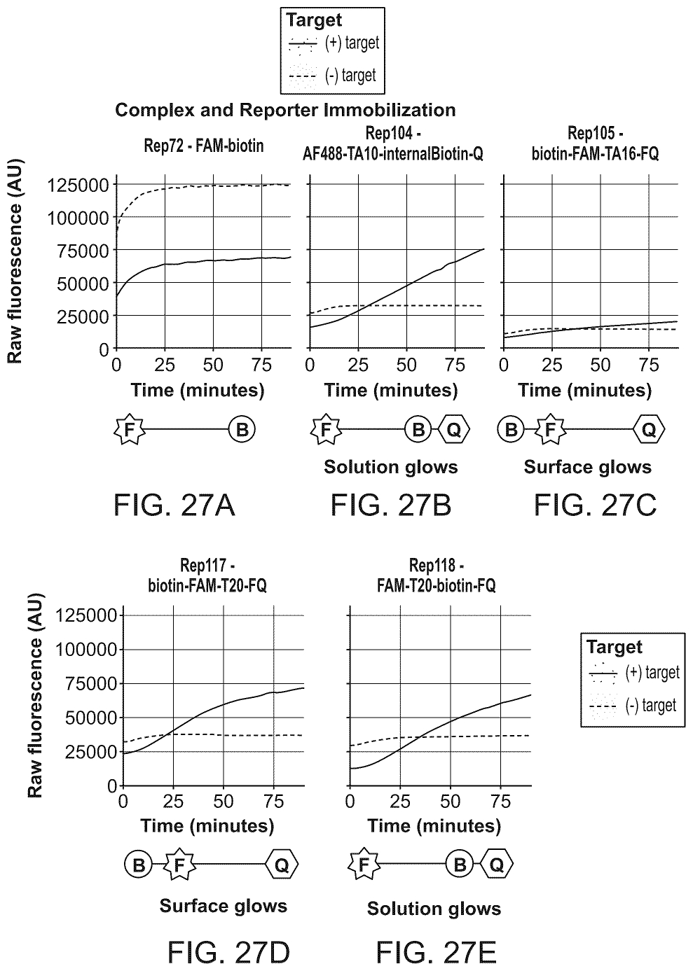

In some embodiments, different reporters are immobilized in combination with Cas complexes on a streptavidin surface for evaluation of the DETECTR assay. FIGS. 27A-27E present results for evaluation of different reporters for immobilization in combination with Cas complex immobilization on a streptavidin surface. In each figure, raw fluorescence is plotted against time in minutes representing kinetic binding curves for each type of reporter while binding with a positive control (+) and negative control (−) target. FIG. 27A presents the binding results for a FAM-biotin reporter, “rep” composed of the fluorophore FAM and biotin and is listed as rep72. The FIG. 27B plots the raw fluorescence for a reporter composed of the fluorophore AlexaFluor488, “AF488,” and TA10-internalBiotinQ. As predicted the positive control shows a positive slope indicating increased binding over the course of the reaction. This is due to the release of FAM dye into solution upon binding and transcleavage. In rep 104 the cleavage point is between the FAM and the biotin, while the biotin in all reporters is the attachment point to the streptavidin surface. FIG. 27C plots the control, target binding kinetic plot for rep 105. Rep105 is composed of biotin-FAM-T16-FQ. In this case the streptavidin coated surface emits fluorescence because the region between the FAM dye and the quencher is cleaved upon binding and the quencher is released. FIG. 27D plots the control for rep117. Rep117 is composed of biotin-FAM-T20-FQ. In this embodiment, the reporter is cleaved between the FAM dye and the quencher, thus allowing for release of the quencher in the solution upon binding and transcleavage. This in turn, causes the surface to emit fluorescence. FIG. 27E plots the control for rep118. Rep118 is composed of FAM-T20-biotin-FQ. In this embodiment, the solution emits fluorescence because upon binding the nucleic acid region between the biotin and the FAM is transcleaved, thus releasing the FAM into solution.

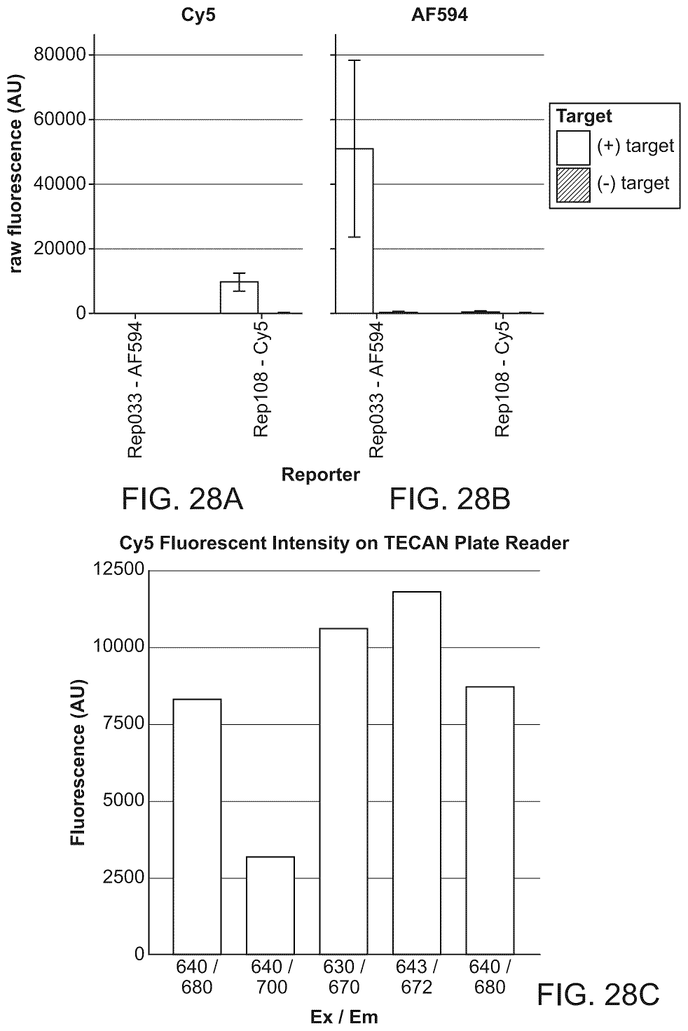

In some embodiments, Cy5 dye may be used as a reporter or a component of a reporter. FIGS. 28A-28C present results for the Cy5 reporter (rep 108) showing that it is functional for DETECTR but produces a weaker signal (might be gain related). FIGS. 28A and 28B plot raw fluorescence versus reporter type for channels configured to read Cy5 dye and Alexa Fluor 594 “AF594” dye, respectively. In these plots the average raw fluorescence is shown for each reporter. Reporter, rep033, readout in the “AF594” channel had the most significant fluorescence signal. FIG. 28C plots raw fluorescence for various combinations of excitation and emission wavelengths on a plate reader for Cy5 dye. Under similar assay conditions AF594 exhibits stronger signal than Cy5, but Cy5 is functional. Optimum excitation and emission wavelengths for Cy5 are shown to be 643 nm and 672 nm, respectively.

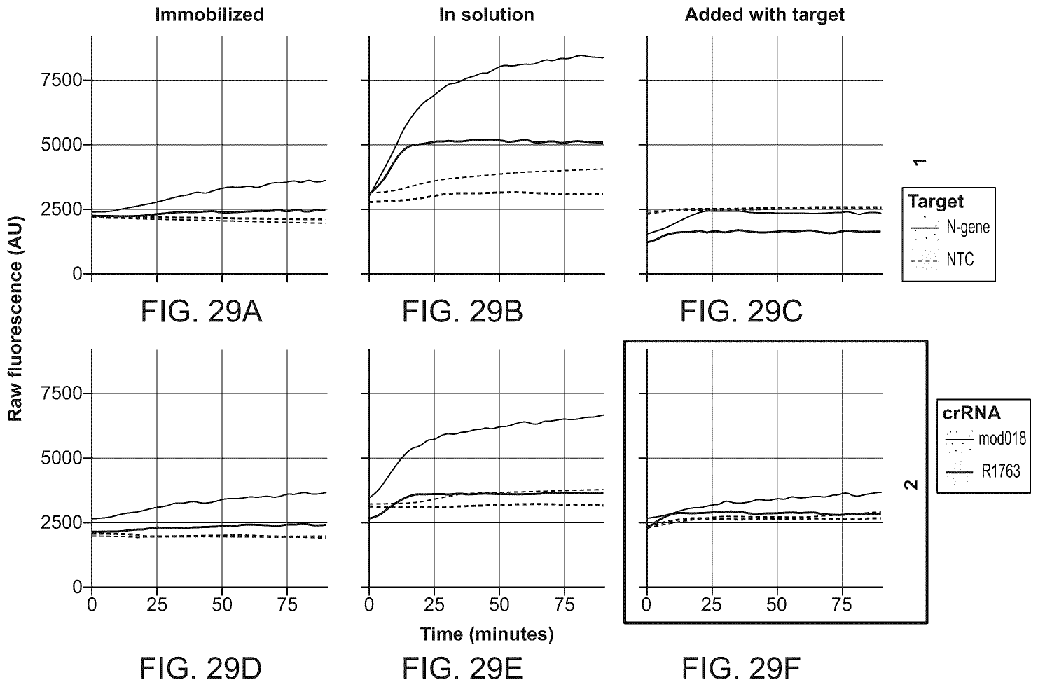

FIGS. 29A-29F present results for optimization of the complex formation step where certain components are immobilized, as described herein. In each figure raw fluorescence is plotted against time in minutes. FIGS. 29A-29C show results for replicate 1. FIGS. 29D-29F show results for replicate 2. In FIGS. 29A and 29D the reporter and gRNA are immobilized. In FIGS. 29B and 29E all components are in solution. In FIGS. 29C and 29F the reporter and gRNA are immobilized and Cas12 and target are added at the same time.

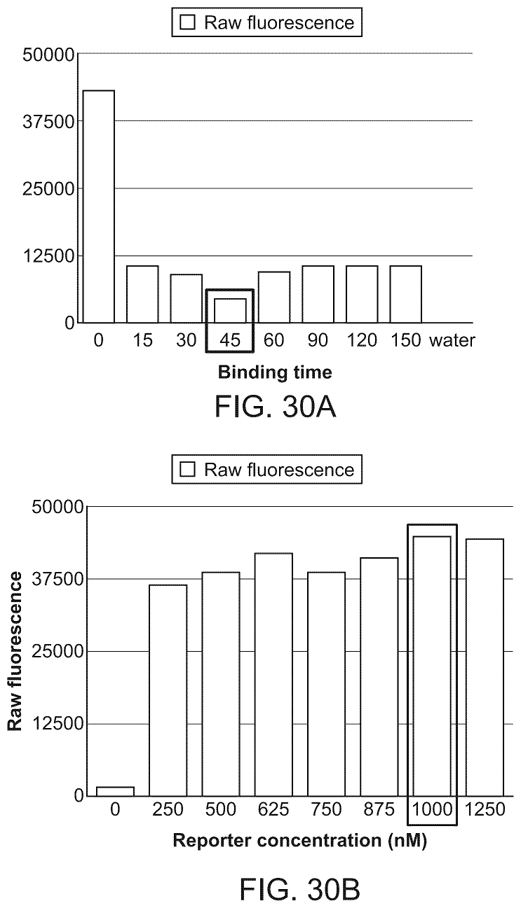

FIGS. 30A-30B present results for immobilization optimization involving a gRNA/reporter binding time and reporter concentration. FIG. 30A is a measurement of supernatant of the surface reaction over time showing the fluorescence dropping and thus indicating uptake of biotinylated dye reporter by the streptavidin surface. In this embodiment a 15 min binding time is found to be sufficient.

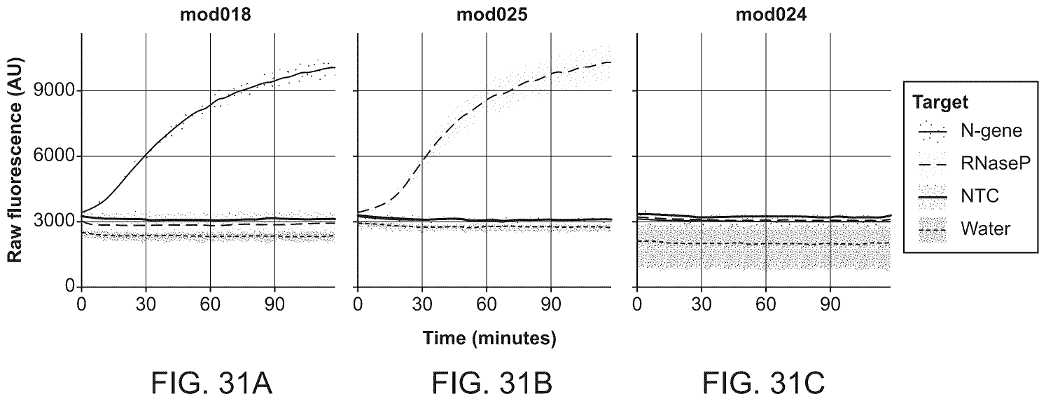

In some embodiments, gRNAs are modified. In some embodiments, the modified gRNAs are modified with linker molecules for immobilization onto a surface. FIGS. 31A-31C present results showing target discrimination of modified gRNAs.

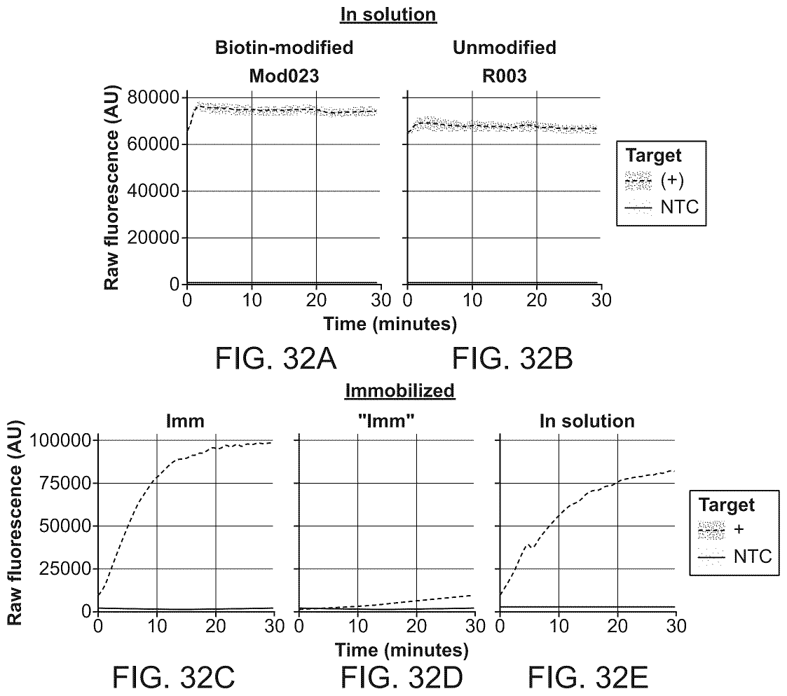

In some embodiments, guide RNAs are modified for surface modification. In some embodiments, reporters are modified for surface immobilization. In these embodiments, an immobilized gRNA, or immobilized reporter or a combination thereof participate in a diagnostic assay including a programmable nuclease. FIGS. 32A-32E present results demonstrating functionality of biotin-modified Cas13a gRNA. In each figure, raw fluorescence is plotted against time in minutes, where the dashed line series represents data for when the target is present, and the thin solid line with low amount of speckle for boundaries series represents when target is not (no target control or NTC). FIGS. 32A and 32B show results, in solution, for mod023, the biotin modified reporter and R003 the non-biotin-modified reporter, respectively. In some embodiments the biotin-modified gRNA has similar performance to the non-biotin-modified gRNA in solution. FIG. 32C shows results for gRNA that was modified with biotin and immobilized to the surface. FIG. 32D shows results for gRNA that was not modified with biotin but was deposited on the surface in the same manner as FIG. 32C. FIG. 32E, similar to FIGS. 32A and 32B, shows results for gRNA that was unmodified and in solution. Together these results showed that with biotin modification and surface immobilization functionality was maintained and DETECTR assay performance was not adversely affected.

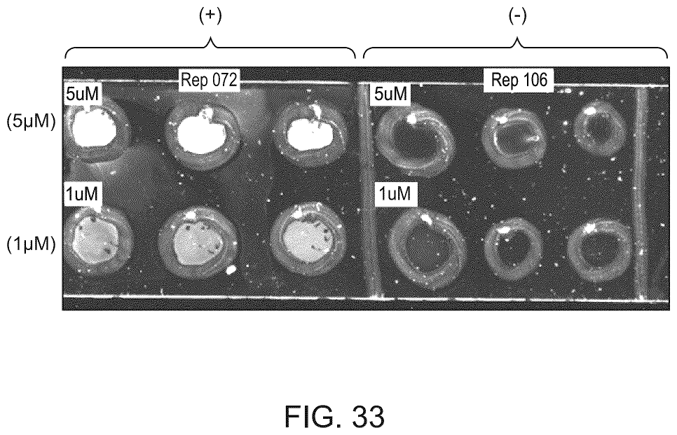

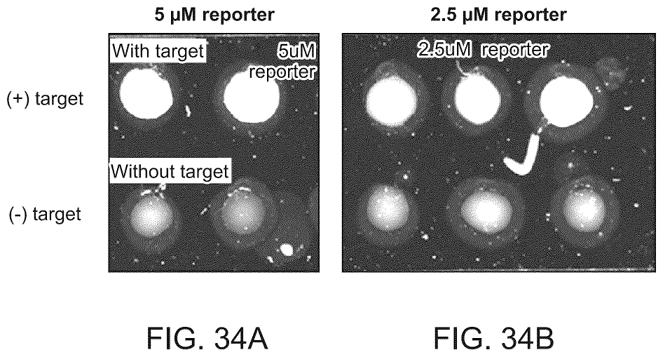

In some embodiments, biomolecules are immobilized to surfaces. In some embodiments, the surfaces were glass. FIG. 33 shows results for the test reporter, Rep072, and the negative control, Rep106. The replicates of Rep072 at 5 μM show the strongest signal and the three replicates of Rep072 at 1 μM concentration show the next strongest signal. The negative control reporter, rep 106 shows the same low signal (on none at all) for both 5 μM and 1 μM concentrations. This result shows specific binding of a FAM-biotinylated reporter with a 30 minute incubation time at both 5 μM and 1 μM concentrations. FIGS. 34A-34B show similar results with reporters at 5 mM concentrations in FIG. 34A and 2.5 mM concentrations in FIG. 34B. The top row of FIGS. 34A and 34B show spots exhibiting bright fluorescence and the bottom row of FIGS. 34A and 34B show spots exhibiting similarly low fluorescence.

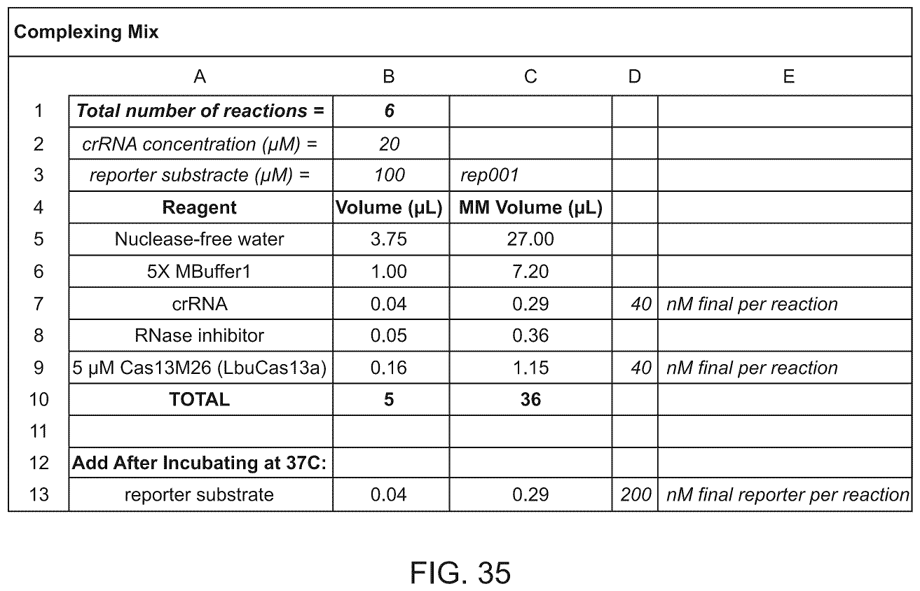

Experimental parameters for the preparation of an embodiment of a complexing mix are seen in FIG. 35.

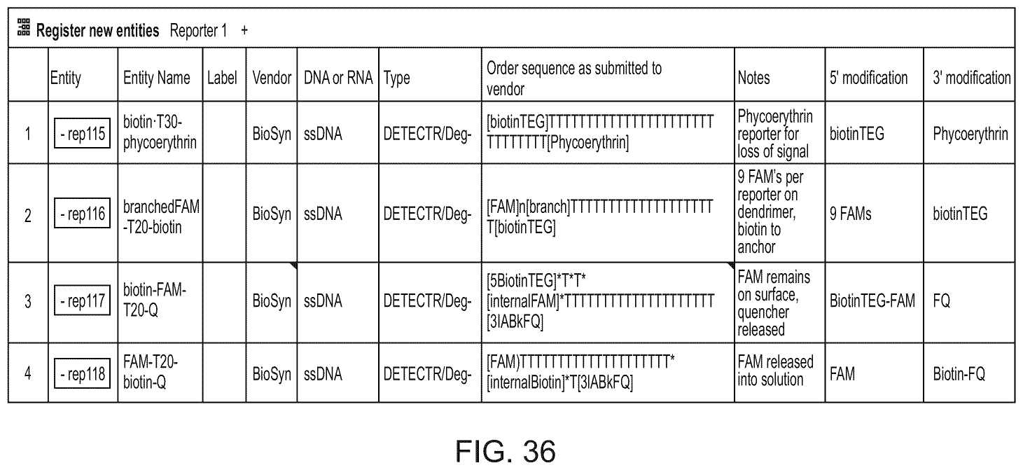

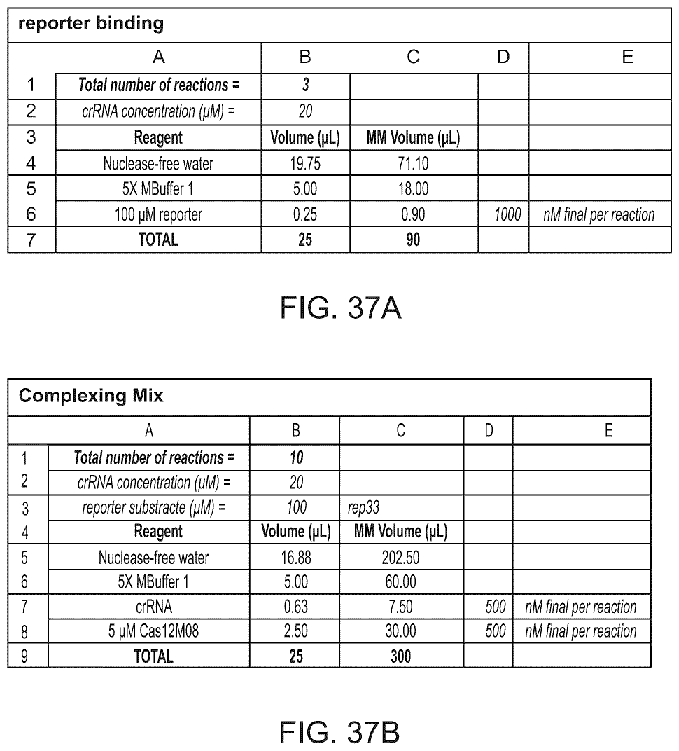

In some embodiments, fluorescent quencher-based reporters are used in the immobilized DETECTR assay. FIG. 36 shows sequence and other details for reporters used in some embodiments. In some embodiments, reporters rep072, rep104, rep105, rep117 and rep118 are used for binding to a reader plate. Reporter binding details and complexing mix parameters as seen in FIGS. 37A and 37B, respectively for some embodiments.

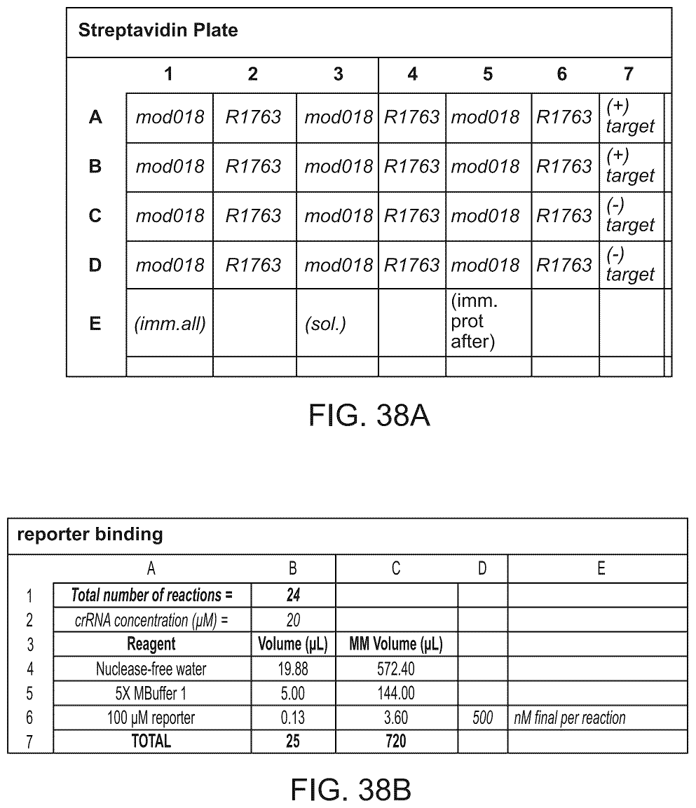

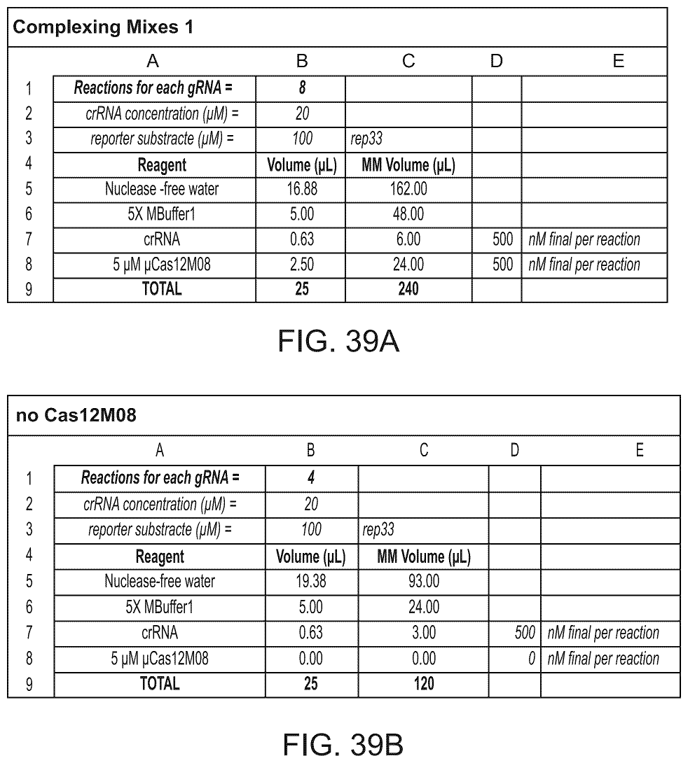

Described herein are various embodiments, where both the gRNA and reporter are bound to a plate as opposed to the gRNA, reporter and CAS protein. This removes the need to functionalize the surface with the pre-complex of gRNA and CAS protein, allowing for an easier manufacturing process. Additionally, greater specificity can be achieved by allowing for more stringent washes. An experimental design of this embodiment and conditions for binding a reporter to a plate in this embodiment are seen in FIGS. 38A and 38B, respectively. Complexing reactions for mod018 (5′ biotin-TEG R1763 SARS-CoV-2 N-gene) and R1763 CDC-N2-Wuhan prepared for a particular embodiment according to the conditions presented in FIG. 39A. Two sets of full complexing mix for an embodiment are seen in FIG. 39B.

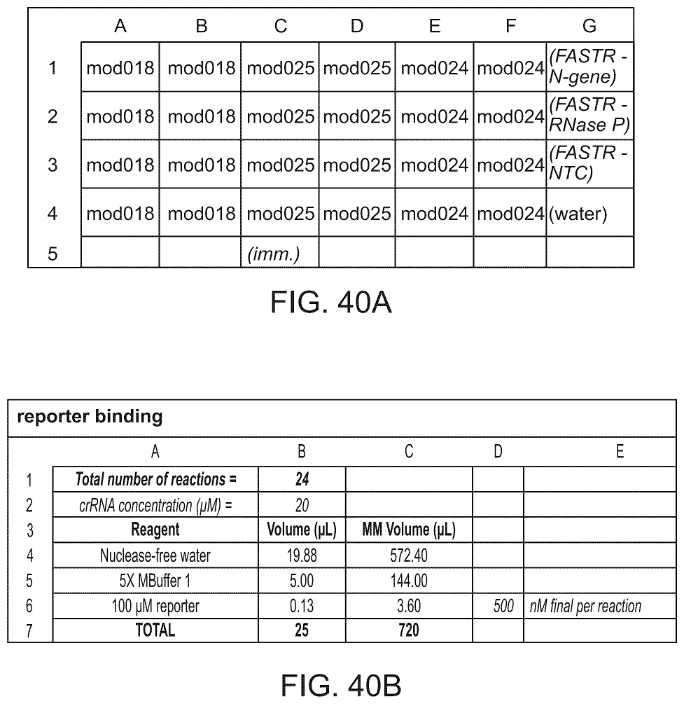

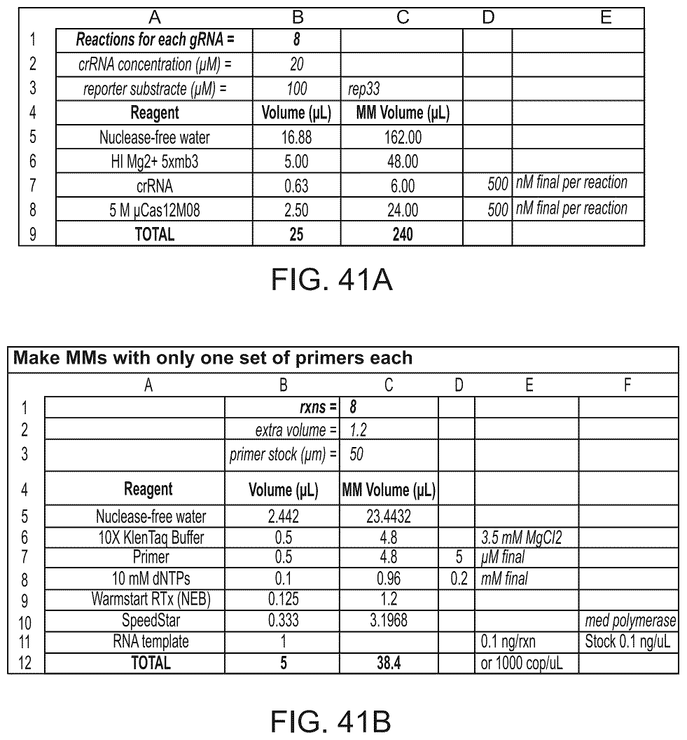

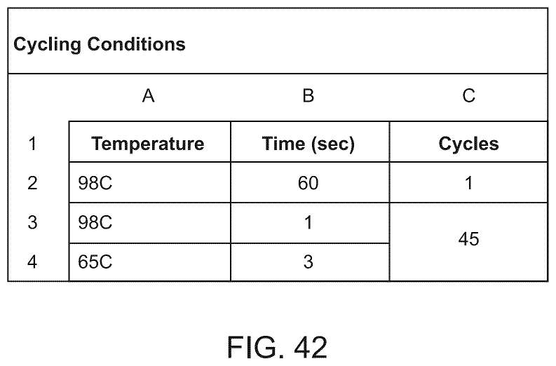

Described herein are various embodiments, that demonstrate target discrimination for immobilized reporters for the DETECTR reaction. An experiment design for such an embodiment is shown in FIG. 40A and reporter binding conditions shown in FIG. 40B. Reaction conditions are shown FIGS. 41A and 41B. PCR conditions are shown in FIG. 42.

In some embodiments, a pneumatic pump interfaces with the cartridge. In some embodiments, as shown in a top down view, in the middle of FIG. 43 of the device may comprise a cartridge with a top layer which may comprise reservoirs. Shown at the bottom of FIG. 43 the device may comprise a sliding valve containing the sample. In some embodiments, the device may comprise a lysis chamber shown at left, followed by amplification chambers to the right, and detection chambers further to the right.

In some embodiments, the DETECTR assay device may comprise a sliding valve as seen in FIG. 44. In some embodiments offset pitch of the channels allows aspirating and dispensing into each well separately and helps to mitigate cross talk between the amplification chambers and corresponding chambers. In some embodiments, a sample moves through the sliding valve device as seen in FIG. 45.

In some embodiments, NHS-Amine chemistry is used for immobilization of DETECTR components. FIG. 83 presents a schematic of combined gRNA and reporter immobilization and results for such embodiments. In this embodiment, a functional DETECTR reaction was immobilized to an NHS modified surface of a support using primary amine modified reporters and gRNAs. In the example shown, a modified reporter (rep111) was bound to the surface in combination with either an unmodified crRNA (R1763) or a modified crRNA (mod027). After incubating these nucleic acids on the surface, the surface was washed 3 times, and then a Cas12 variant (SEQ ID NO: 17) was added with the target dsDNA. The immobilized DETECTR reaction was then incubated in a plate reader at 37 C for 60 minutes with continuous monitoring of the fluorescence.

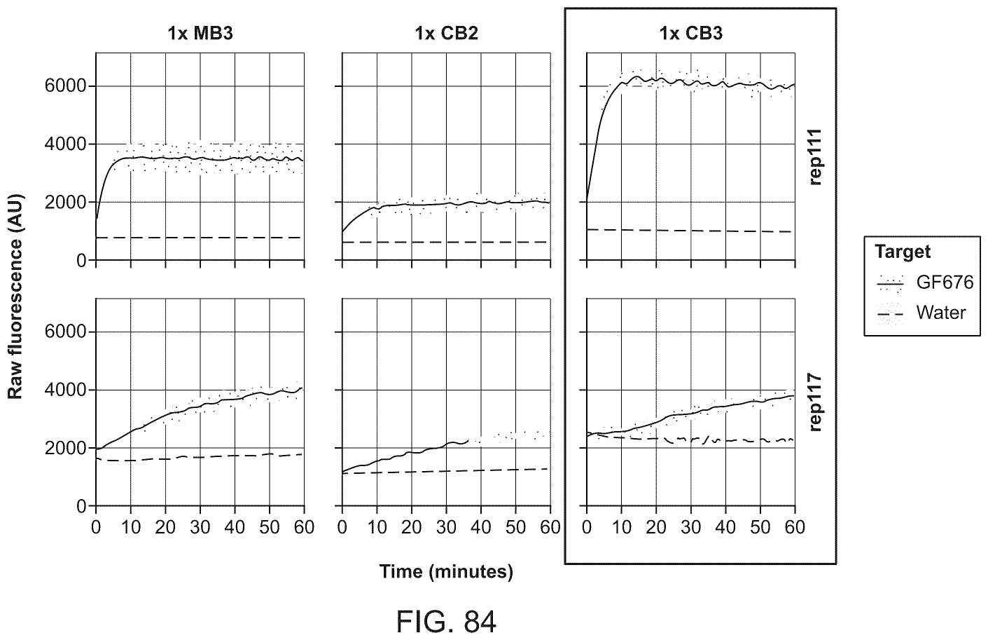

FIG. 84 presents results for various embodiments involving the optimizing conjugation buffer to reduce non-specific binding. For some embodiments, 1× Conjugation Buffer 3 (CB3) was selected as the buffer to perform binding studies. I was found that CB3 improved the binding of the amine-modified reporter (rep111) and reduced the binding of a biotinylated reporter (rep117) which should not bind to NHS covalently. In some embodiments, the wash buffer used was 1× MB3. In some embodiments, 1× MB3: 20 mM HEPES, pH 7.2, 2 mM KOAc, 5 mM MgAc, 1% Glycerol, 0.0016% Triiton X-100 was used. In some embodiments, 1× CB2: 20 mM HEPES, pH 8.0, 2 mM KOAc, 5 mM MgAc, 1% Glycerol, 0.0016% Triiton X-100 was used. In some embodiments, 1× CB3: 100 mM HEPES, pH 8.0, 10 mM KOAc, 25 mM MgAc was used.

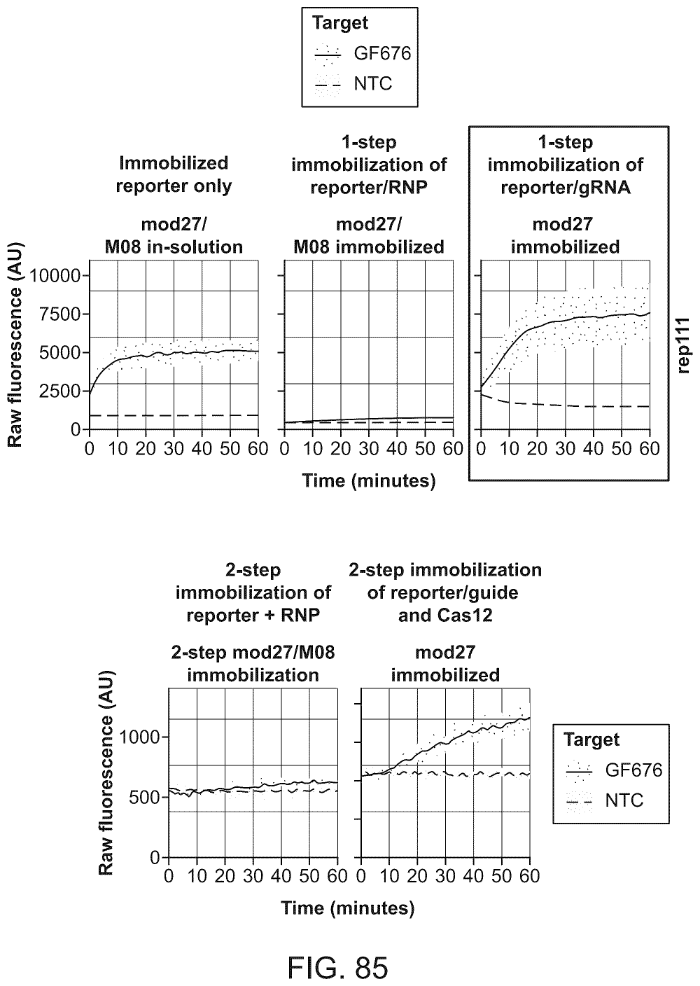

In some embodiments, different combinations of reporter+guide+a Cas12 variant (SEQ ID NO: 17) are immobilized. FIG. 85 presents results of such embodiments, involving the optimization the assay. For such embodiments, it was found that immobilizing guide and reporter first followed by the addition of a Cas12 variant (SEQ ID NO: 17) and target at the same time gave sufficient signal.

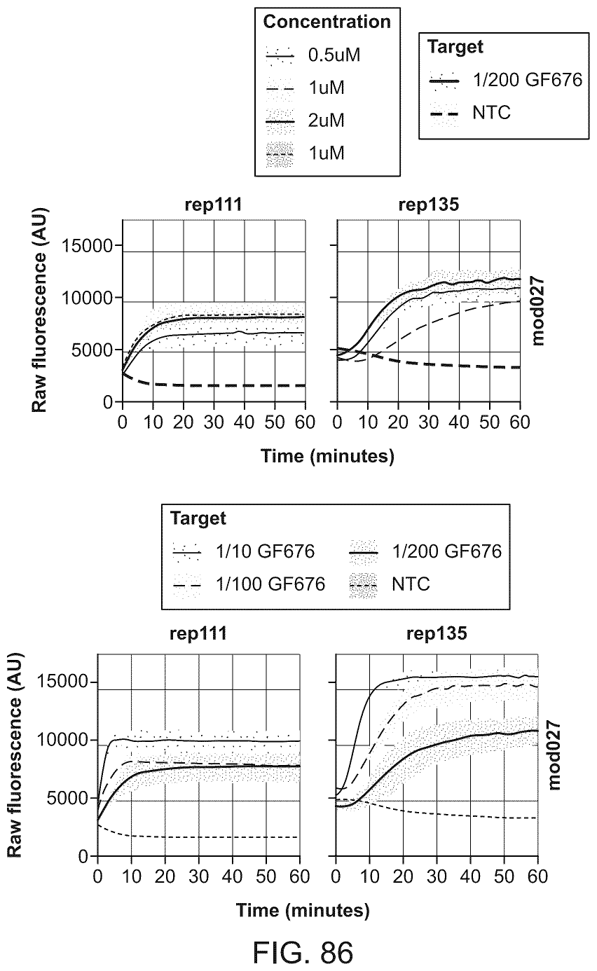

The results for optimizing gRNA and target concentrations to improve single-to-noise ratio for immobilized DETECTR assay are shown in FIG. 86. In some embodiments, guide concentration is increased while keeping reporter concentration constant at 0.5M, as seen on the left of FIG. 86, In such embodiments, the signal is not changed very much. In some embodiments, as seen on the right of FIG. 86, increasing target concentration 2-fold helped improve the overall signal with rep135. Additionally, for such embodiments, rep135 gave a better signal strength compared to rep111. The sequences for the two reporters are: rep111: 5AmMC6T//16-FAMK/TTTTTTTTTTTT/3IABKFQ/(SEQ ID NO: 1490) and rep 135: 5AmMC12//16-FAMK/TTTTTTTTTTTTTTTTTTT/3IABKFQ/(SEQ ID NO: 1491).

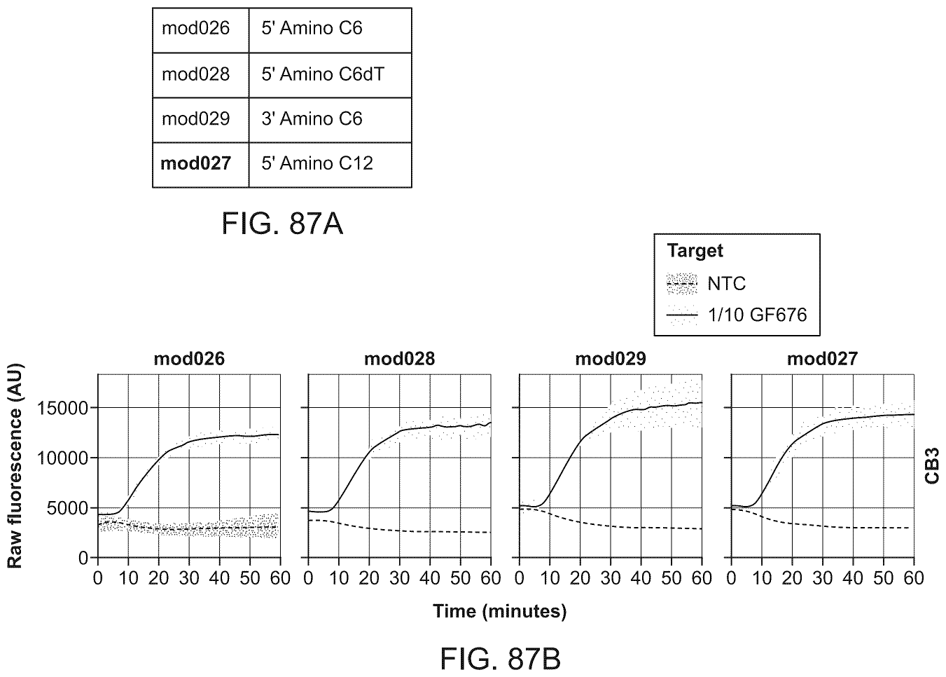

In some embodiments, amino modifications are used for DETECTR immobilization. FIG. 87 presents results for such embodiments. For each subplot raw fluorescence is plotted against time in minutes. Each of the four subplots represent different modifications. The solid line trace represents the no target control (NTC) and the dashed line trace represents a 1:10 dilution of target-GF676.

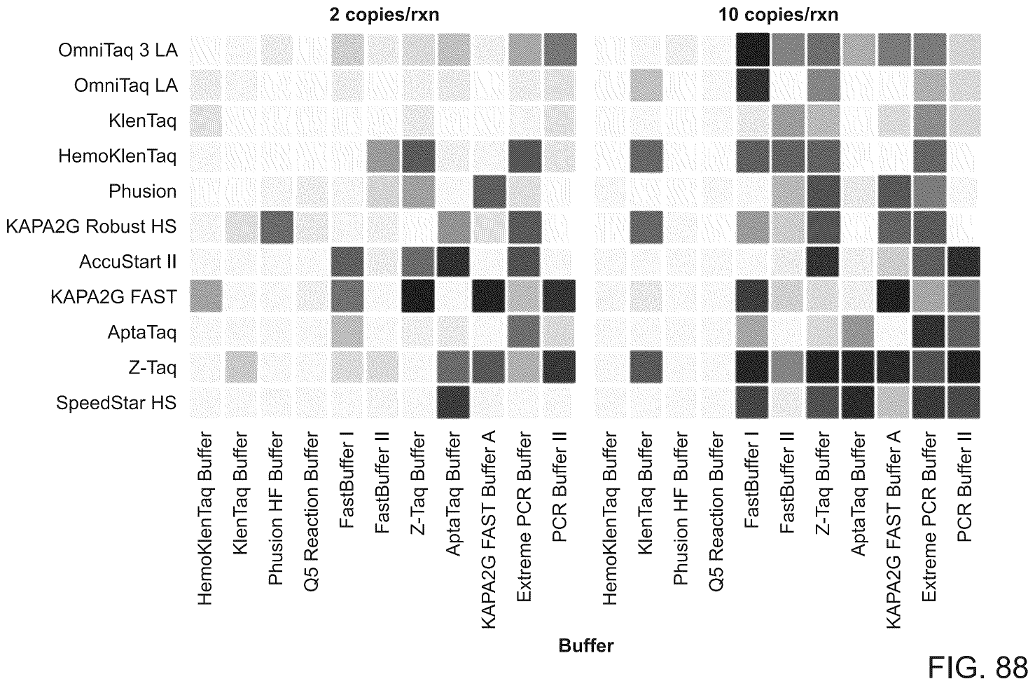

In some embodiments, rapid thermocycling and CRISPR diagnostics are used to detect SARS-CoV-2. Results are shown in FIG. 88. For some embodiments, polymerase and buffer combinations were identified that enabled the rapid amplification of SARS-CoV-2 using the N2 primers from the CDC assay. The assay of such embodiments was performed at two target concentrations: 2 copies/reaction and 10 copies/reaction. In some embodiments, reaction conditions are as follows: initial denaturation at 98 C for 30 seconds, followed by 45 cycles consisting of 1 second at 98 C and 3 seconds at 65 C. Following thermocycling, amplicon was transferred to a Cas12 variant (SEQ ID NO: 17) detection reaction for 30 minutes at 37° C. Best performing enzyme/buffer pairs shown in FIG. 88 were those that gave strong signal in both tested concentrations.

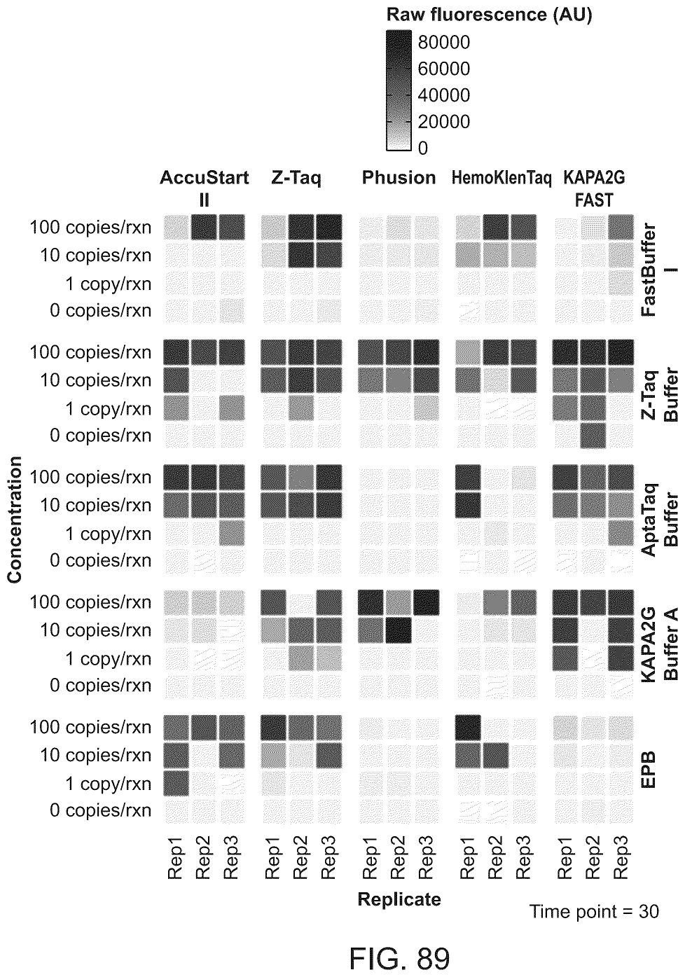

The top enzymes and buffers identified previously at various concentrations and with multiple replicates were tested for the FASTR assay. In some embodiments, the best performing enzymes and buffers as identified in the previously disclosed screening studies were used. Results of such embodiments are shown in FIG. 89. Reaction conditions of such embodiments are as follows: initial denaturation at 98 C for 30 seconds, followed by 45 cycles consisting of 1 second at 98 C and 3 seconds at 65 C. Primers used were from the CDC N2 assay for SARS-CoV-2. Following thermocycling, amplicon was transferred to a Cas12 variant (SEQ ID NO: 17) detection reaction for 30 minutes at 37 C. The data present is the signal from the CRISPR reaction. Best performing enzyme/buffer pairs were those that gave strong signal at the lowest tested concentrations and with detection across replicates.

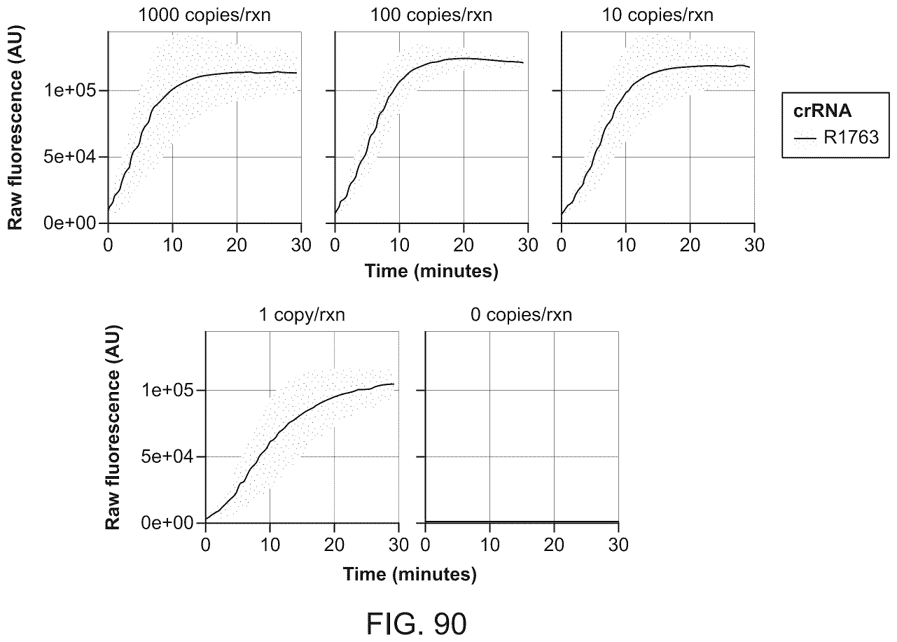

For some embodiments, single copy detection of SARS-CoV-2 with FASTR assay has been demonstrated as shown in FIG. 90. In some embodiments, the limit of detection of the FASTR assay was evaluated using solutions composed of 1000 copies of SARS-CoV-2 per reaction to 1 copy per reaction. For some embodiments, reaction conditions are as follows: reverse transcription at 55 C for 60 seconds, initial denaturation at 98 C for 30 seconds, followed by 45 cycles consisting of 1 second at 98 C and 3 seconds at 65 C. Primers used were from the CDC N2 assay for SARS-CoV-2. Following thermocycling, amplicon was transferred to a Cas12 variant (SEQ ID NO: 17) detection reaction for 30 minutes at 37 C. The data presented in FIG. 90 is the signal from the CRISPR reaction. It was found that the limit of detection of the CRISPR assay was 1 copy of SARS-CoV-2 per reaction.

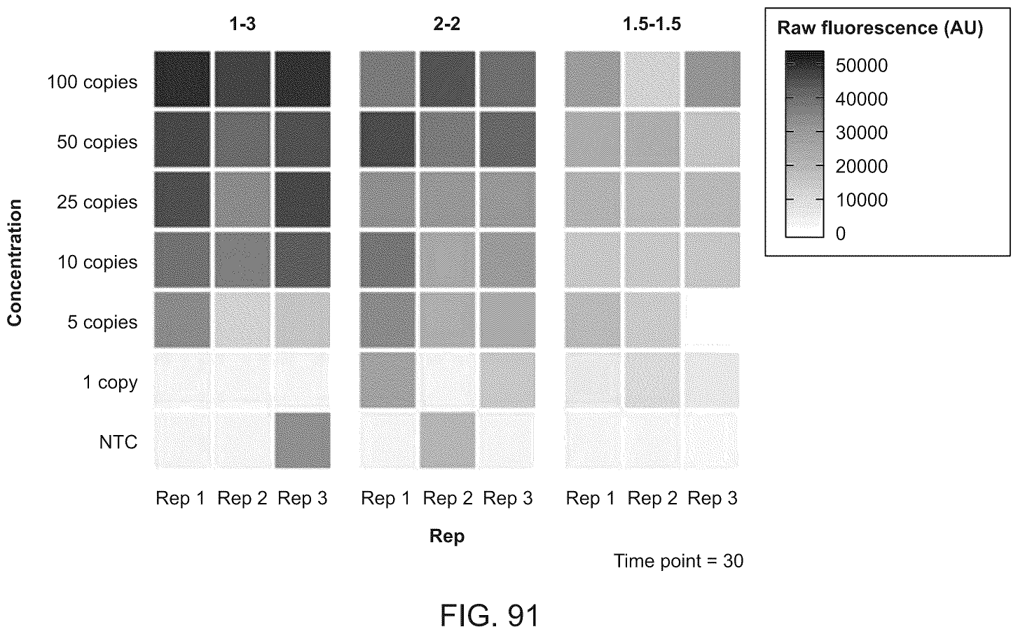

Variations on Across some embodiments, rapid cycling times are varied to evaluate denaturation and annealing/extension for the FASTR assay. Results for such embodiments are shown in FIG. 91. In some embodiments, reverse transcription is run at 55C for 60 seconds and initial denaturation at 98 C for 30 seconds. In some embodiments, the tested cycling conditions were: 98 C for 1 second, 65 C for 3 seconds; 98 C for 2 seconds, 65 C for 2 seconds; or 98 C for 1.5 seconds, 65 C for 1.5 seconds. In some embodiments, primers used were from the CDC N2 assay for SARS-CoV-2. Following thermocycling, amplicon was transferred to a Cas12 variant (SEQ ID NO: 17) detection reaction for 30 minutes at 37 C. The results shown in FIG. 91 indicate that >2 seconds of annealing/extension time at 65 C are necessary for robust sensitivity.

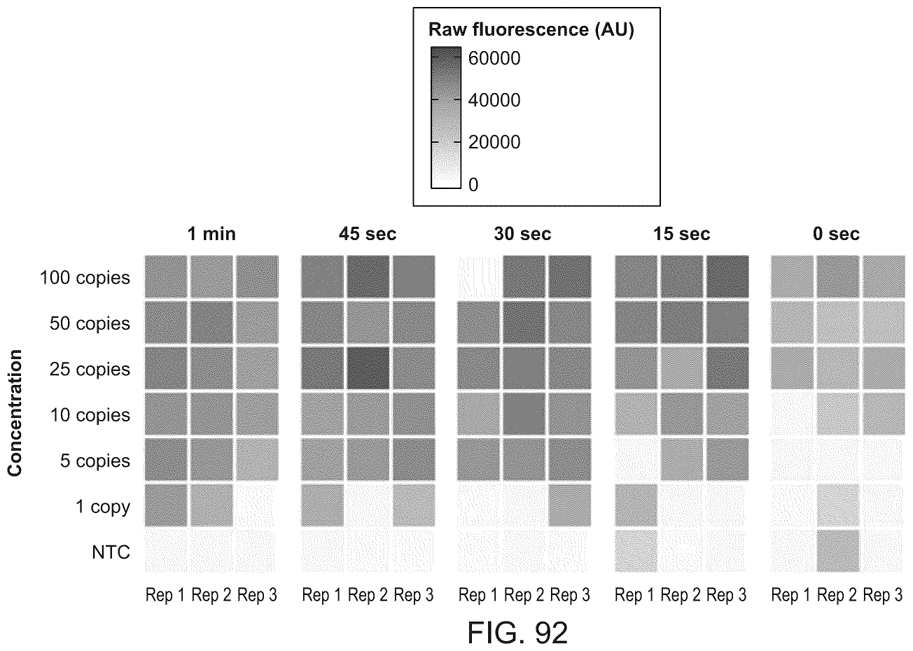

FIG. 92 presents results for Minimizing RT time for FASTR. The performance of the FASTR assay was evaluated, for various embodiments, where the reverse transcription incubation times were varied holding temperature at 55 C. The results shown in FIG. 92 indicate the assay is most robust above 30 seconds of reverse transcription.

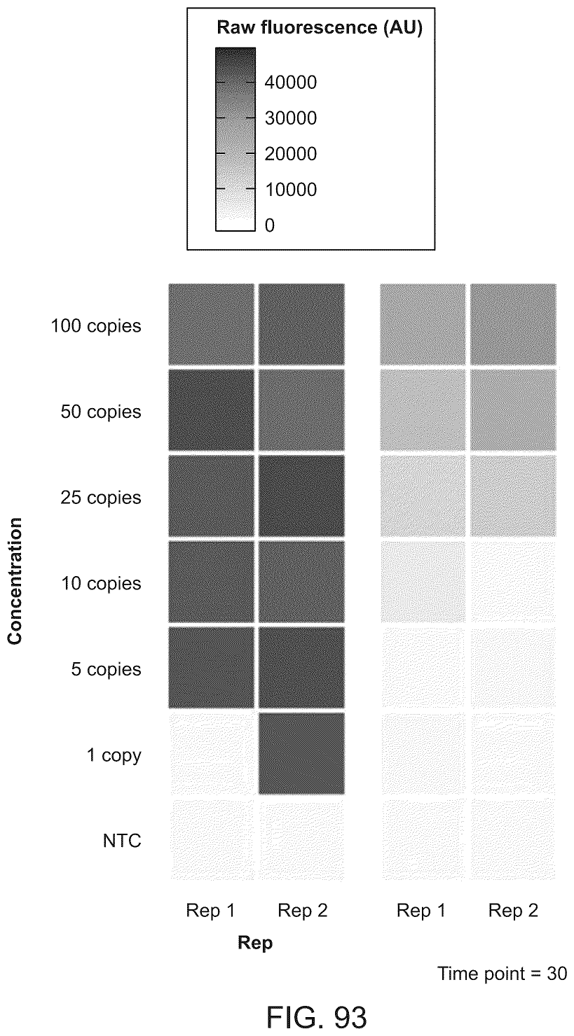

FIG. 93 presents results for Higher pH buffers improve FASTR performance. In some embodiments, the FASTR assay utilizes buffers with pH of either 9.2 or pH 7.8. The FASTR assay was evaluated using these buffer pH values. The results as shown in FIG. 93 indicate that the higher pH buffer produced superior results in terms of amplicon yield and sensitivity.

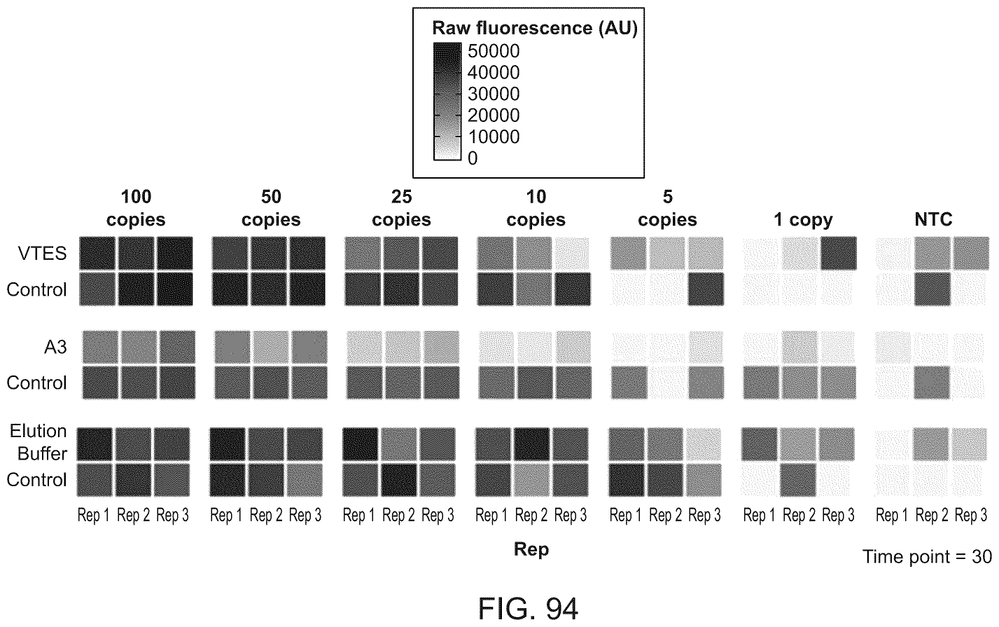

For some embodiments, the FASTR assay compatibility with crude lysis buffers was investigated. Results are shown in FIG. 94 where there are three row groups, each consisting of two sub rows representing a buffer and control from top to bottom respectively. The buffers, VTES, A3 and Elution buffer are plotted against a control, from top to bottom, respectively. In FIG. 94 there are also 7 subgroups showing the number of copies decreasing from left to right. For certain embodiments, the performance of the FASTR assay when combined with various crude lysis buffers was evaluated, where crude lysis buffers VTE5, A3, and the Elution Buffer from the ChargeSwitch kit (Thermo) were tested. For certain embodiments, the FASTR assay performed best for VTE5, but was performed slightly less robustly in the A3 buffer and the Elution Buffer from the ChargeSwitch kit performed similarly to the control reactions (water).

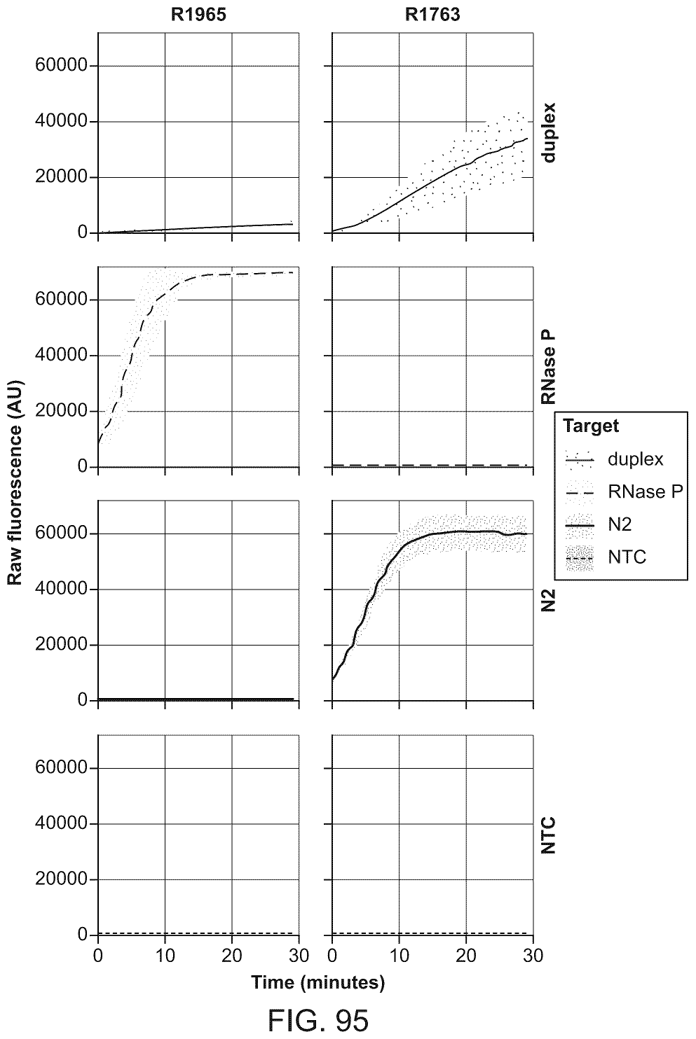

For some embodiments non-optimized multiplexing of FASTR was demonstrated as shown in FIG. 95. In FIG. 95, raw fluorescence is plotted in the y-axis and time is plotted in the x-axis for each sub-plot. Each column illustrates a particular sequence: R1965 and R1763, respectively Each row represents duplex, RNase P, N2 and the no target control, from top to bottom respectively. For some embodiments, initial testing of multiplexed FASTR for SARS-CoV-2 and RNase P POP7 (endogenous control) showed that while the single-plex assays generated a robust signal in DETECTR, the duplex assay tended to generate a weak signal for SARS-CoV-2 (R1763) and almost no signal for RNase P (R1965). In some embodiments, reaction conditions were as follows: reverse transcription at 55 C for 60 seconds, initial denaturation at 98 C for 30 seconds, followed by 45 cycles consisting of 1 second at 98 C and 3 seconds at 65 C. In some embodiments, primers from the CDC N2 assay for SARS-CoV-2, and M3637/M3638 were used.

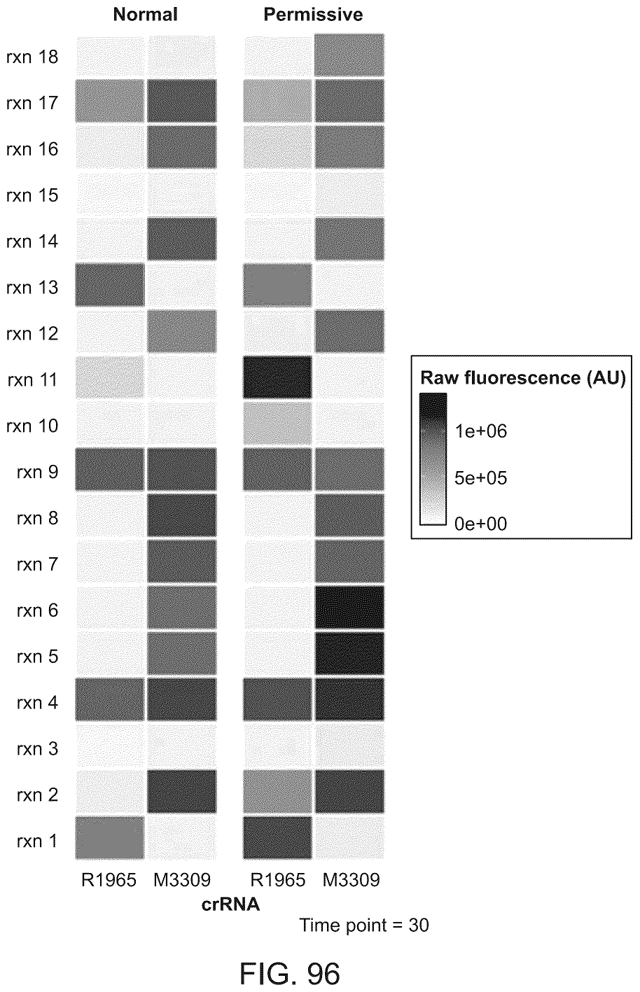

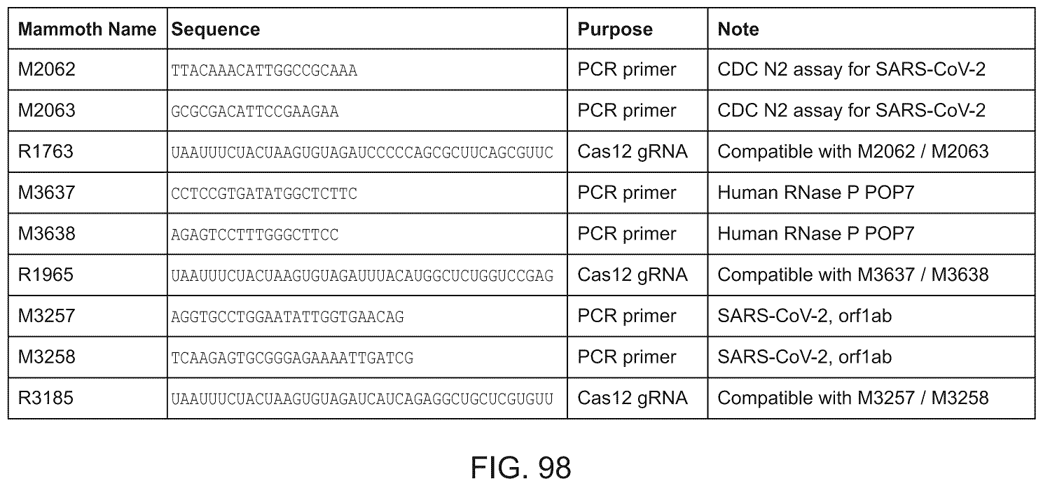

In various aspects described herein, FASTR can be used for multiplexed detection, as shown in FIG. 96. The components of a FASTR reaction, such as primer concentration, dNTP concentration, presence/absence of DMSO, and other factors, can impact the performance of a multiplex FASTR reaction. FIG. 98 shows 18 different experimental conditions for a multiplex FASTR reaction targeting human RNase P POP7 and SARS-CoV-2. In FIG. 96 each row of the y-axis represents experimental runs 1-18 and each column represents the detection signal from a particular crRNA at a time point of 30 minutes in the reaction. The color represents the value of the raw fluorescence. In some embodiments, the multiplexed FASTR assay for SARS-CoV-2 and RNase P, comprise a set of SARS-CoV-2 primers (M3257/M3258). A series of experiments of such embodiments was performed with varied reaction conditions containing different combinations of buffers, primer concentrations, dNTPs, and DMSO. Results identified two reaction conditions that performed robustly for the multiplex reaction. In one of these embodiments, reaction 4, conditions consisted of: 1× FastBuffer 2; 1 μM RNase P primers; 0.5 μM CoV primers; 0.2 mM dNTPs; and 2% DMSO. In another embodiment, reaction 9, conditions consisted of: 1× Klentaq1 buffer; 1 μM RNase P primers; 0.5 μM CoV primers; 0.4 mM dNTPs; and 0% DMSO. In some embodiments, normal reaction conditions consisted of reverse transcription at 55 C for 60 seconds, initial denaturation at 98C for 30 seconds, followed by 45 cycles consisting of 1 second at 98 C and 3 seconds at 65 C. In various aspects, permissive reaction conditions consisted of reverse transcription at 55 C for 60 seconds, initial denaturation at 98 C for 30 seconds, followed by 45 cycles consisting of 3 seconds at 98 C and 5 seconds at 65 C.

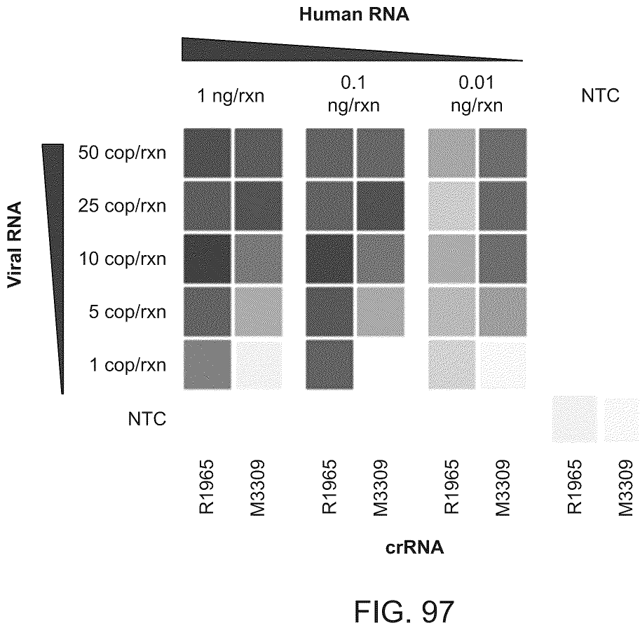

In some embodiments, the FASTR assay enables multiplexed detection. Results of a limit of detection (LOD) study of such embodiments are shown in FIG. 97. In FIG. 97, the x-axis shows the number of copies per reaction for viral RNA and the y-axis of each subplot identifies the particular crRNA. Each subplot shows nanograms of human RNA per reaction, decreasing in concentration from left to right. The 4th subplot contains no human RNA, labeled as the no target control (NTC). For some embodiments, an optimized multiplexed FASTR assay was ran at various concentrations of human RNA and viral RNA. In some embodiments, results indicated that the assay performed at a range of human RNA concentrations, while maintaining a sensitivity of ˜5 copies per reaction. In certain aspects, results shown are from DETECTR reactions using either R1965 to detect the human RNase P, or R3185 (labeled M3309) to detect SARS-CoV-2. In various aspects, reaction conditions are as follows: reverse transcription at 55C for 60 seconds, initial denaturation at 98 C for 30 seconds, followed by 45 cycles consisting of 1 second at 98 C and 3 seconds at 65 C. In some embodiments, primers used were M3257/M3258 (SARS-CoV-2) and M3637/M3638 (RNase P).

In some embodiments, key primers and gRNAs have the sequences as listed in FIG. 98.

### Sample Preparation and Lyophilization

Described herein are various methods of sample preparation and reagent storage. Any of the devices described herein may comprise one or more sample preparation reagents Any of the devices described herein may comprise sample preparation reagents as dried reagents. Dried reagents may comprise solids and/or semi-solids. In certain instances, dried reagents may comprise lyophilized reagents. Any of the devices described herein may comprise one or more lyophilized reagents (e.g., amplification reagents, programmable nucleases, buffers, excipients, etc.). In certain instances, methods include sample lysis, concentration, and/or filtration. In certain instances, methods include reconstitution of one or more lyophilized reagents. In some embodiments, lyophilized reagents may be in the form of lyophilized beads, spheres, and/or particulates. In some embodiments, the lyophilized bead, sphere, and/or particulate may comprise either single or multiple compounds. In some embodiments, the lyophilized bead, sphere, and/or particulate may be adjusted to various moisture levels or hygroscopy. In some embodiments, the lyophilized bead, sphere, and/or particulate may comprise assay internal standards. In some embodiments, the lyophilized bead, sphere, and/or particulate may have diameters between about 0.5 millimeters to about 5 millimeters in diameter.

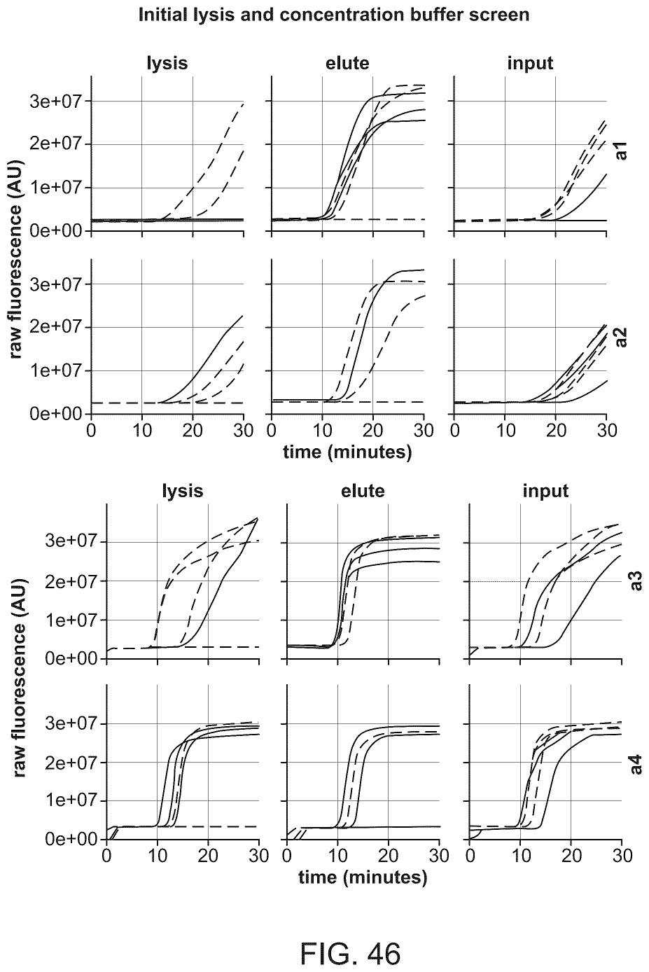

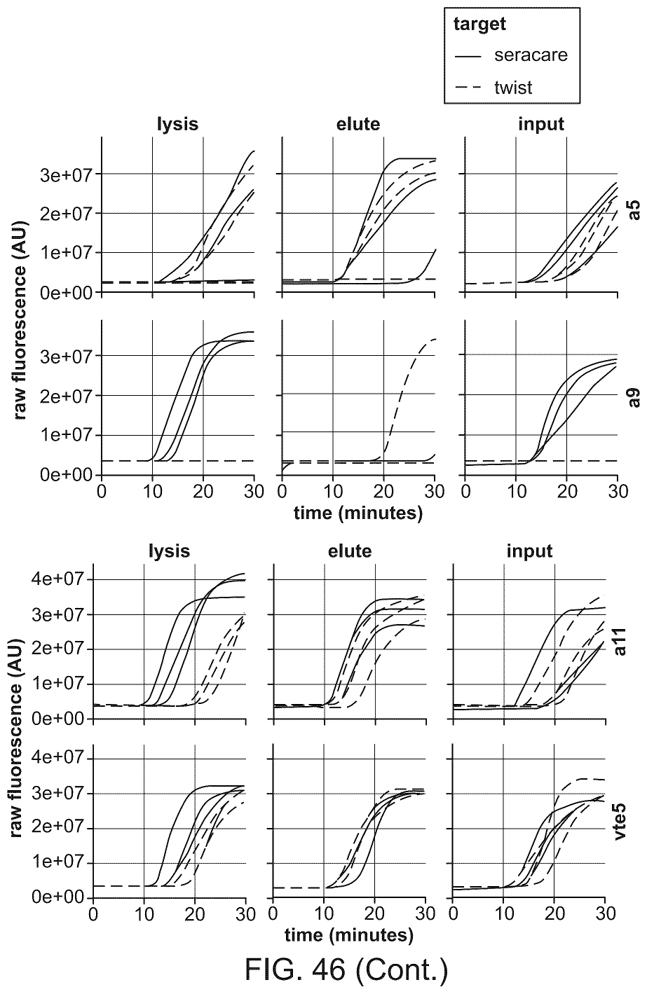

Described herein are various embodiments of the DETECTR reaction involving optimization of sample preparation and lyophilization. Such embodiments allow for adapting the buffer for binding a substrate to perform a concentration step. In some embodiments, experiments may be performed to evaluate the lysis (sample is evaluated directly in the assay) and binding (the sample is eluted from magnetic beads) characteristics of buffers with different components. In such embodiments, the input sample is the same concentration as the eluted sample. Results showing strong lysis activity, but minimal binding/concentration potential are shown in FIG. 46. Buffers a1, a3 and potentially all of such embodiments show acceptable binding activity. Buffer a5, of such embodiments, shows modest activity for both lysis and binding and buffer a2 is not suitable for either activity.

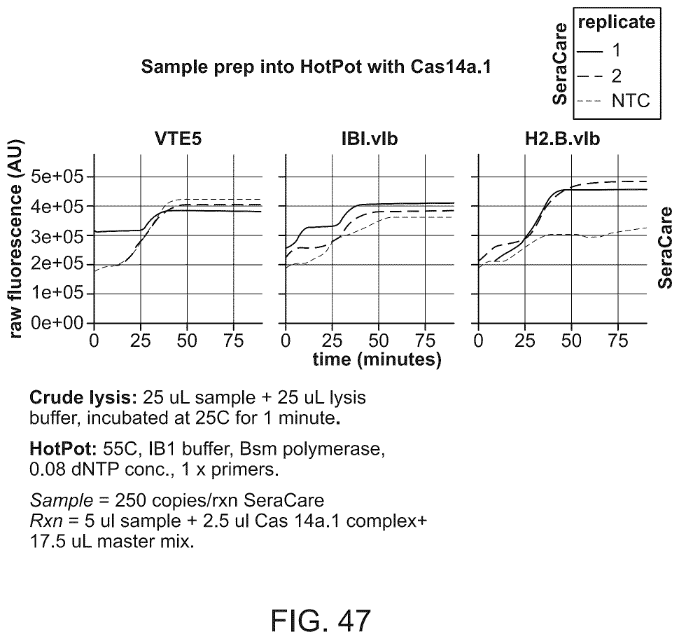

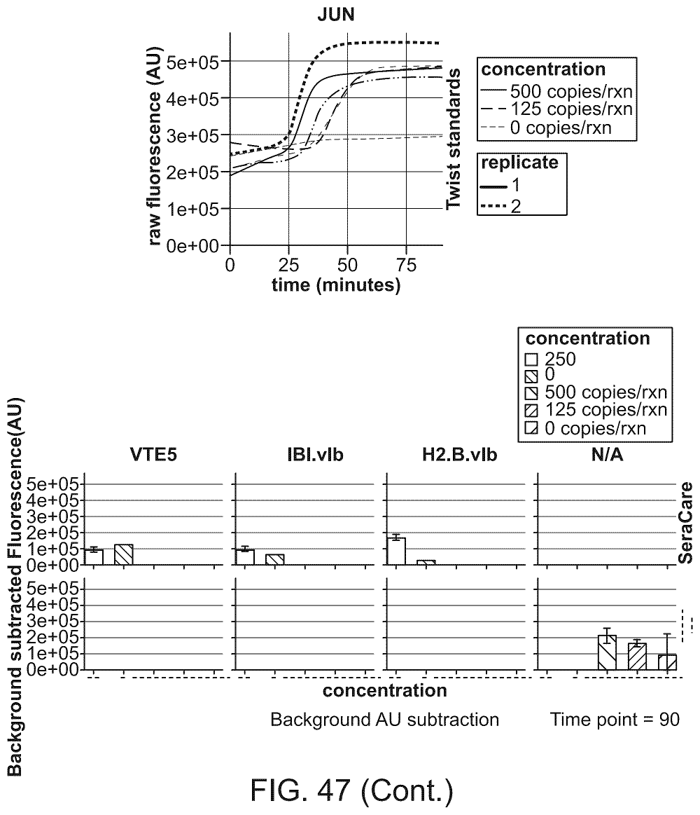

In some embodiments, crude lysis buffer is used in a one-pot assay with Cas14a.1. Results can be seen in FIG. 47.

In some embodiments, the enzyme Cas12 variant (SEQ ID NO: 17) is used with the DETECR assay. In some embodiments, the sequence of the Cas12 variant is: MKKIDNFVGCYPVSKTLRFKAIPIGKTQENIEKKRLVEEDEVRAKDYKAVKKLIDRYHR EFIEGVLDNVKLDGLEEYYMLFNKSDREESDNKKIEIMEERFRRVISKSFKNNEEYKKIFS KKIIEEILPNYIKDEEEKELVKGFKGFYTAFVGYAQNRENMYSDEKKSTAISYRIVNENM PRFITNIK VFEKAKSILDVDKINEINEYILNNDYYVDDFFNIDFFNYVLNQKGIDIYNAIIG GIVTGDGRKIQGLNECINLYNQENKKIRLPQFKPLYKQILSESESMSFYIDEIESDDMLID MLKESLQIDSTINNAIDDLKVLFNNIFDYDLSGIFINNGLPITTISNDVYGQWSTISDGWNE RYDVLSNAKDKESEKYFEKRRKEYKKVKSFSISDLQELGGKDLSICKKINEIISEMIDDYK SKIEEIQYLFDIKELEKPLVTDLNKIELIKNSLDGLKRIERYVIPFLGTGKEQNRDEVFYGY FIKCIDAIKEIDGVYNKTRNYLTKKPYSKDKFKLYFENPQLMGGWDRNKESDYRSTLLR KNGKYYVAIIDKSSSNCMMNIEEDENDNYEKINYKLLPGPNKMLPKVFFSKKNREYFAP SKEIERIYSTGTFKKDTNFVKKDCENLITFYKDSLDRHEDWSKSFDFSFKESSAYRDISEF YRDVEKQGYRVSFDLLSSNAVNTL VEEGKLYLFQLYNKDFSEKSHGIPNLHTMYFRSLF DDNNKGNIRLNGGAEMFMRRASLNKQDVTVHKANQPIKNKNLLNPKKTTTLPYDVYK DKRFTEDQYEVHIPITMNKVPNNPYKINHMVREQLVKDDNPYVIGIDRGERNLIYVVVV DGQGHIVEQLSLNEIINENNGISIRTDYHTLLDAKERERDESRKQWKQIENIKELKEGYIS QVVHKICEL VEKYDAVIALEDLNSGFKNSRVKVEKQVYQKFEKMLITKLNYMVDKKK DYNKPGGVLNGYQLTTQFESFSKMGTQNGIMFYIPAWLTSKMDPTTGFVDLLKPKYKN KADAQKFFSQFDSIRYDNQEDAFVFKVNYTKFPRTDADYNKEWEIYTNGERIRVFRNPK KNNEYDYETVNVSERMKELFDSYDLLYDKGELKETICEMEESKFFEELIKLFRLTLQMR NSISGRTDVDYLISPVKNSNGYFYNSNDYKKEGAKYPKDADANGAYNIARKVLWAIEQ FKMADEDKLDKTKISIKNQEWLEYAQTHCE* (SEQ ID NO: 17). The * indicates a stop codon.

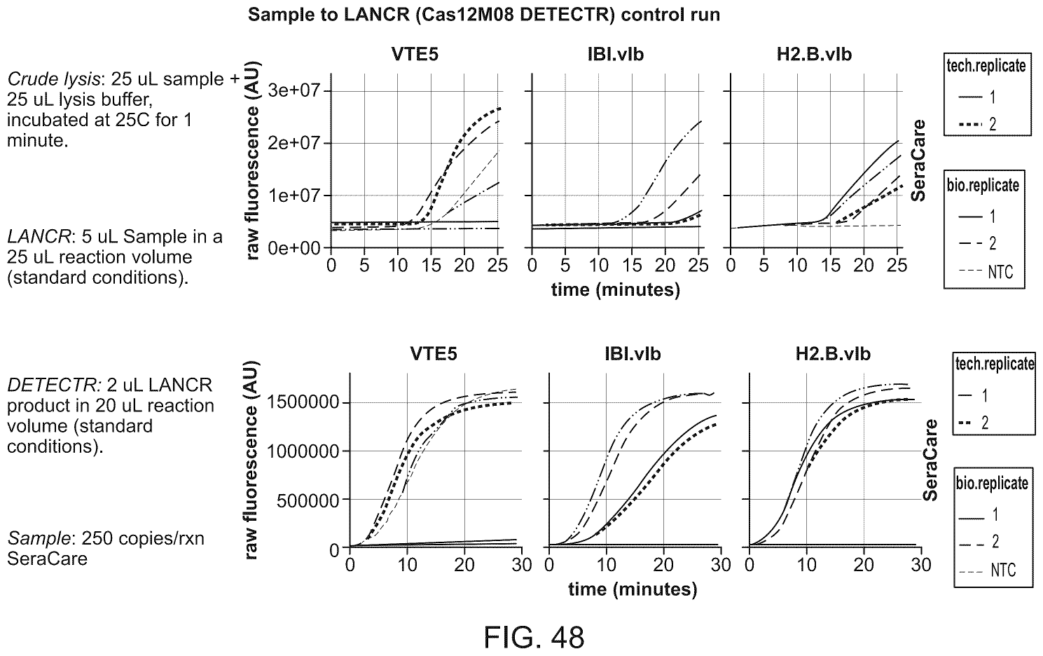

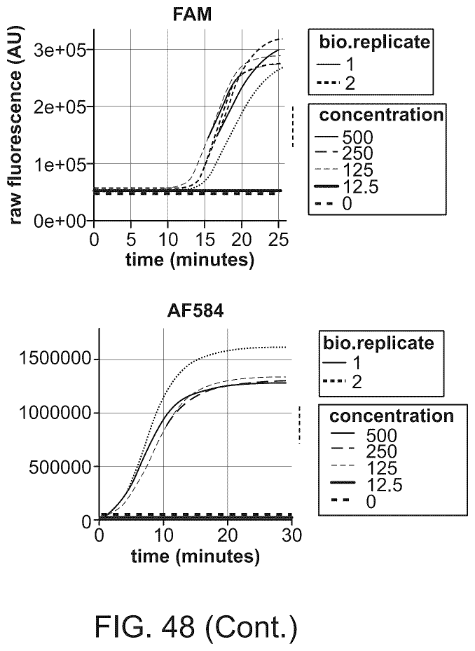

FIG. 48 presents results for a control study involving sample preparation optimization of the LANCR (Cas12 variant DETECTR) assay using SEQ ID NO: 17. In such embodiments, crude lysis involves: 25 uL sample+25 uL lysis buffer and incubation at 25 C for 1 minute. In some embodiments, the LANCR reaction is run as follows: 5 uL sample in a 25 uL reaction volume (standard conditions). In some embodiments the DETECTR reaction is run as follows: 2 uL LANCR product in 20 uL reaction volume (standard conditions). Sample: 250 copies/reaction SeraCare.

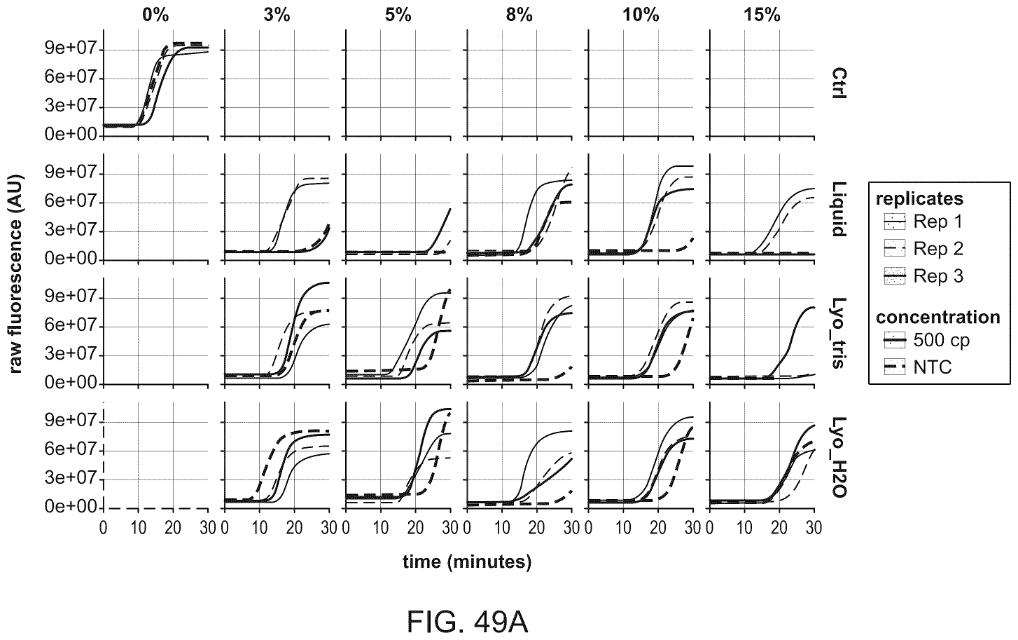

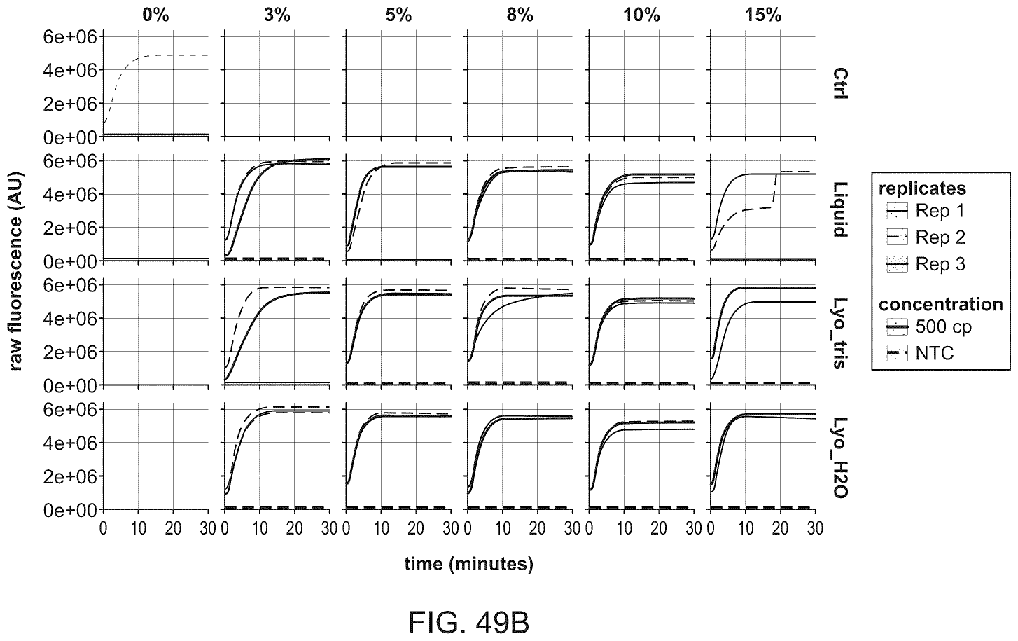

For some embodiments, two groups of conditions were evaluated for lyophilization performance. For one embodiment, Group I, Trehalose is used. FIGS. 49A-49B presents lyophilization optimization results for the Group 1 using RT-LAMP assay is presented in FIG. 49A and the DETECTR assay as seen in FIG. 49B.

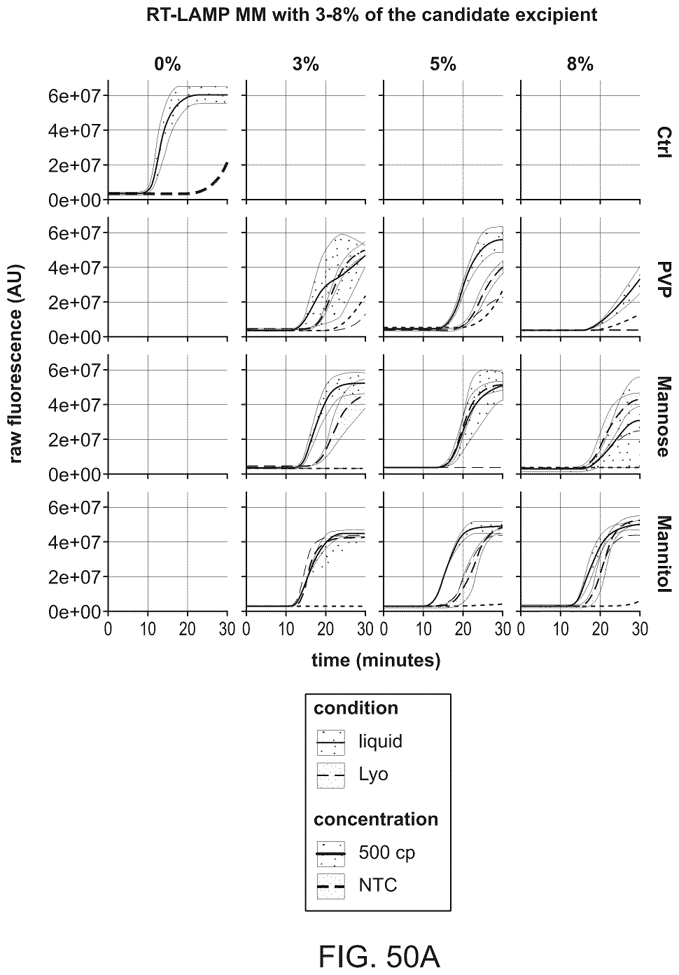

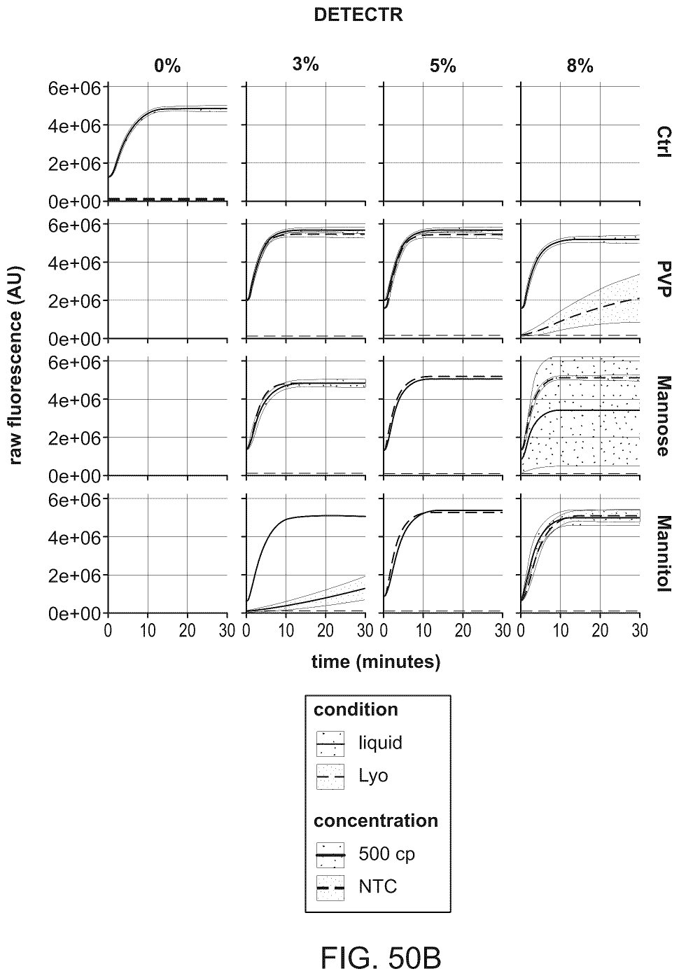

In another embodiment, Group II comprising: PVP 40; sorbitol; Mannitol; and Mannosse are used. FIGS. 50A-50B presents lyophilization optimization results for Group 2 evaluated using RT-LAMP 3-8% of the candidate excipient as shown in FIG. 50A and for Group 2 using the DETECTR assay conditions as seen in FIG. 50B.

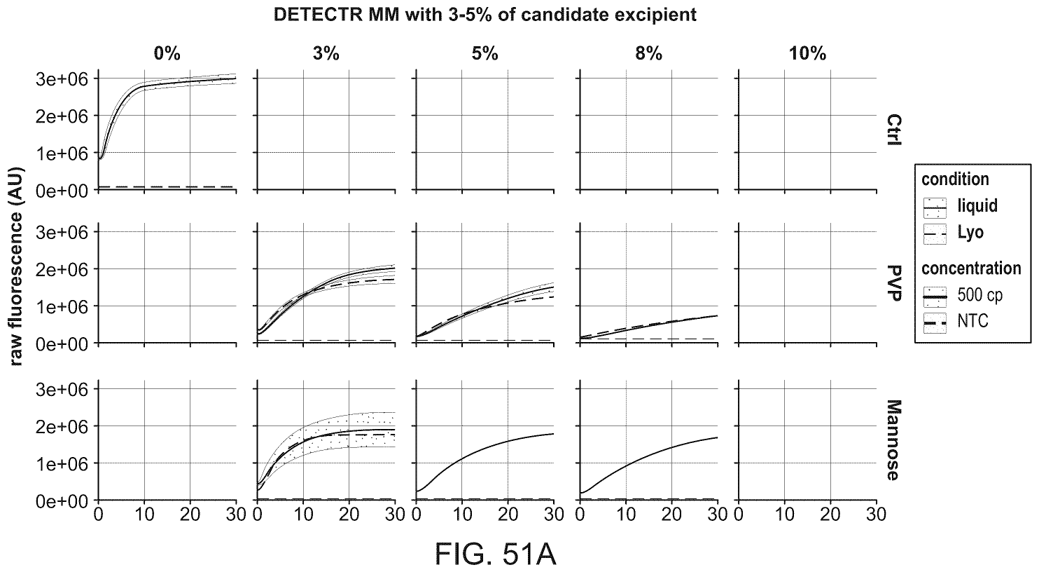

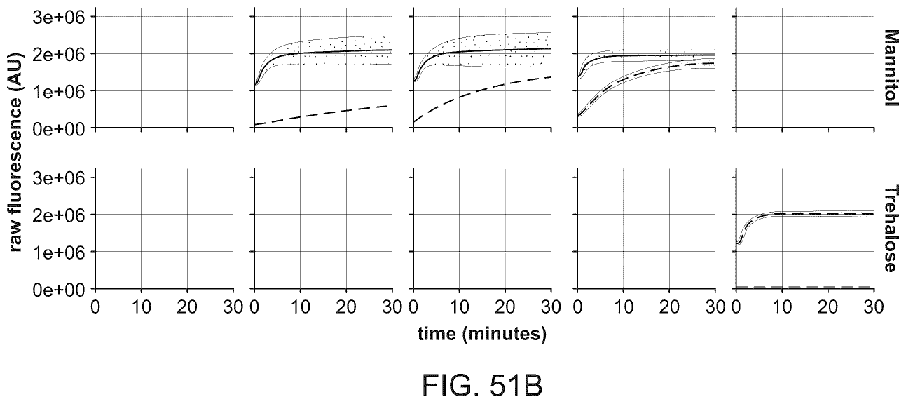

FIGS. 51A-51B presents lyophilization optimization results for an embodiment of Group 2: PVP 40, Sorbitol, Mannitol, Mannose, using DETECTR MM with 3-5% of candidate excipient.

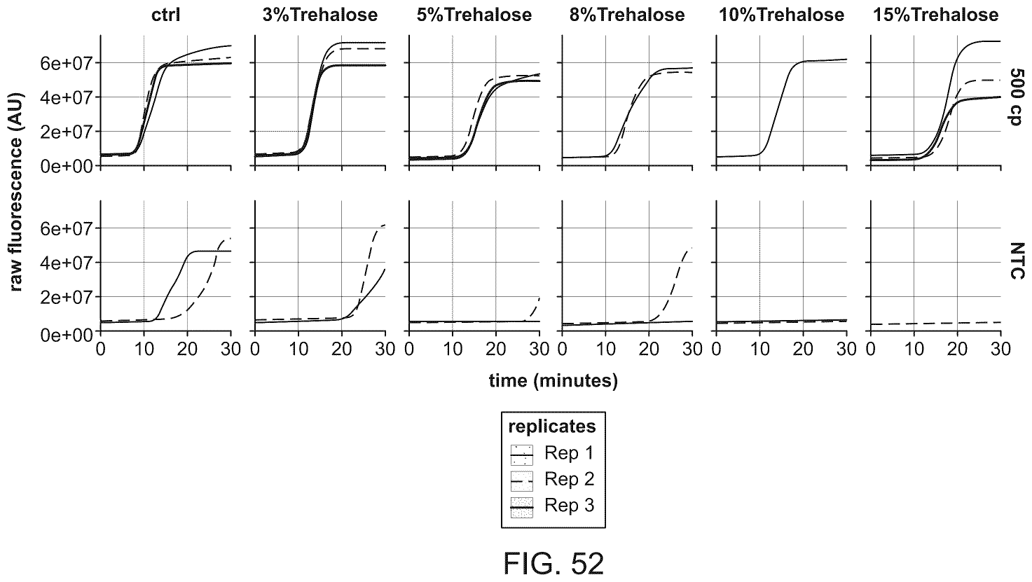

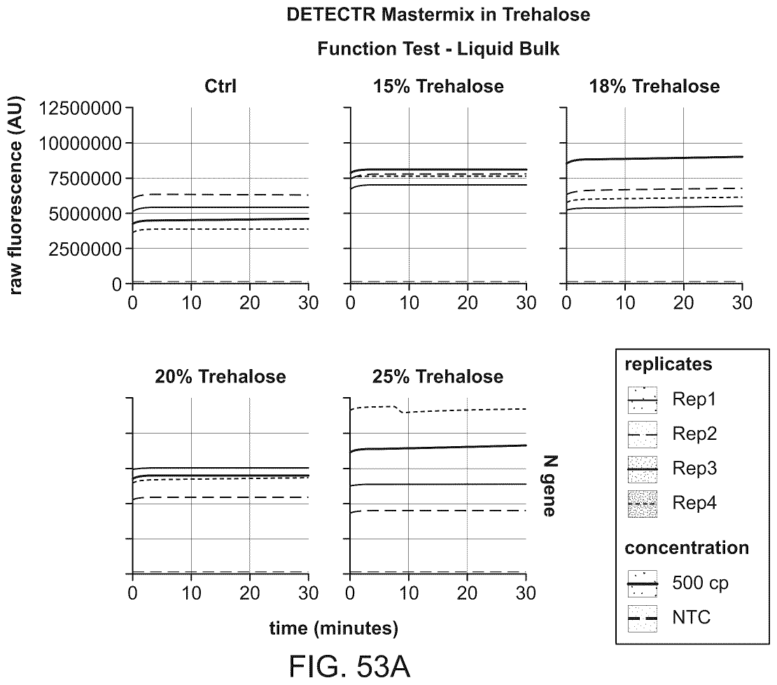

In some embodiments, Trehalose is used to control the rate of the reaction. FIG. 52 presents results from a study involving such embodiments, where RT-LAMP reaction components and various percentages of Trehalose were used.

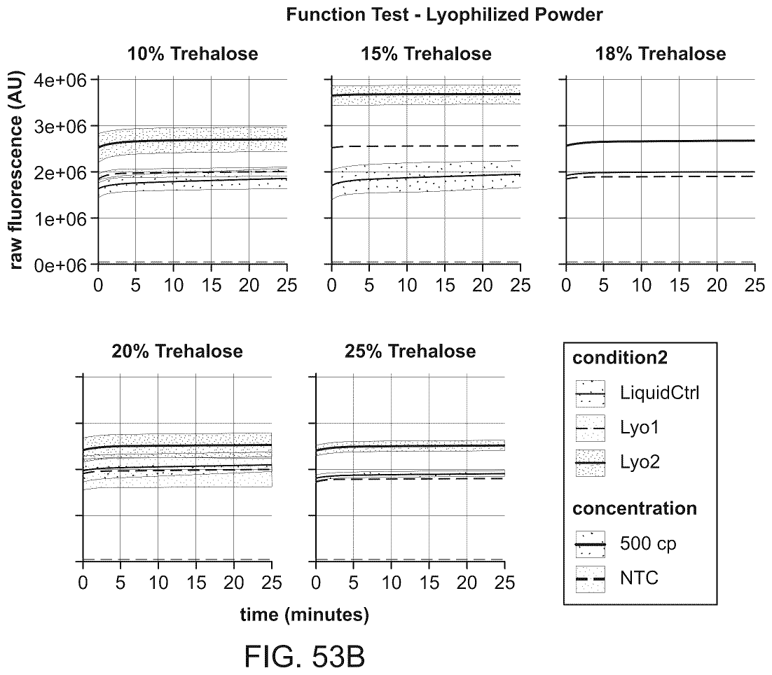

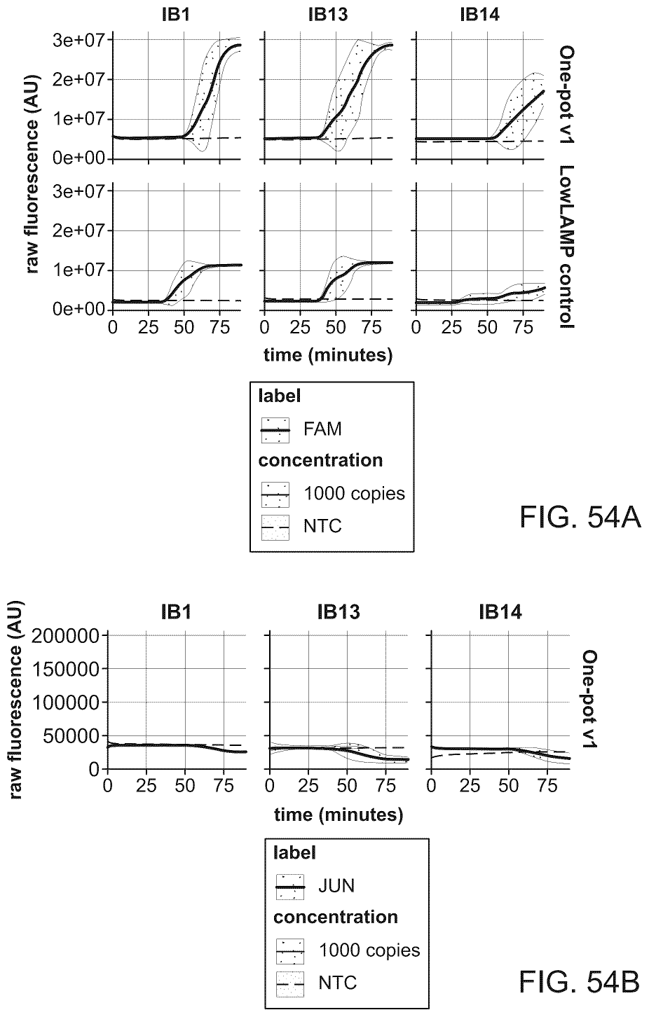

FIGS. 53A-53B presents results of a study involving embodiments where DETECTR reaction components and Trehalose are used. The top row of subplots show results for increasing Trehalose percentages in liquid bulk solution. The bottom row show results for increasing Trehalose in lyophilized powder form. In such embodiments, primary drying lasts 90 hours, fill volume is 250 uL in a 2 mL vial; reconstituted with 250 uL water; secondary drying is 6 hours; and temperature ramp is 0.5° C. per minute.

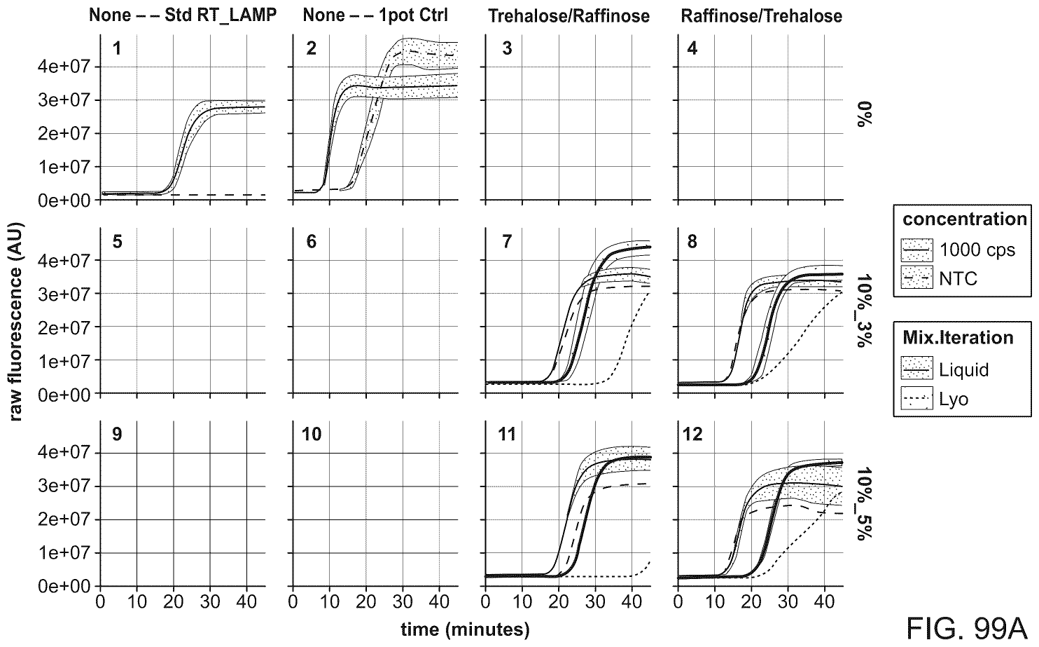

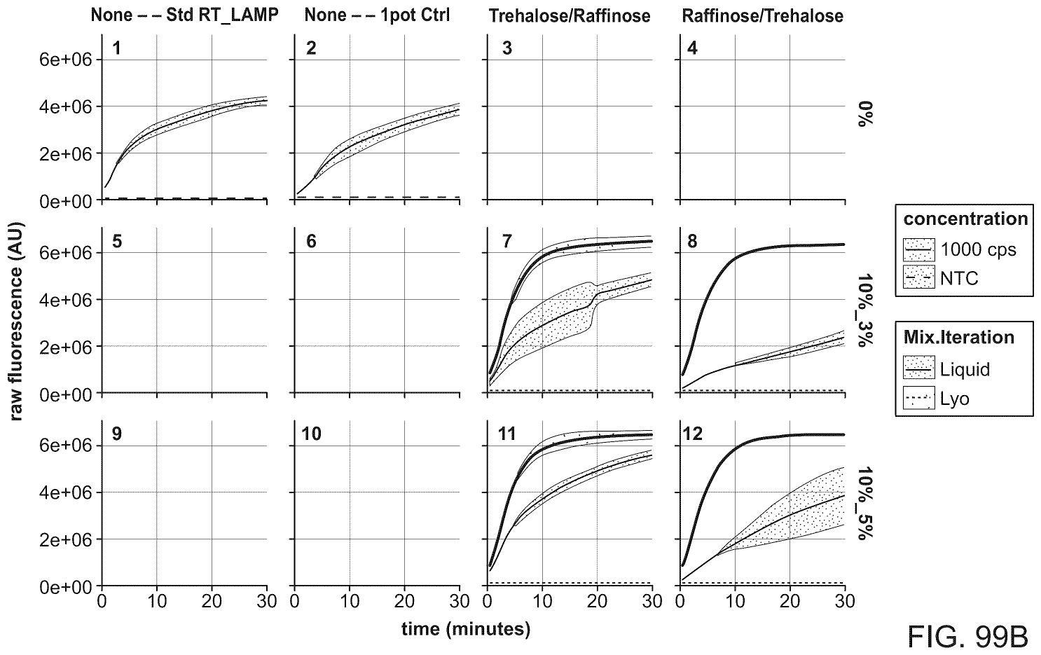

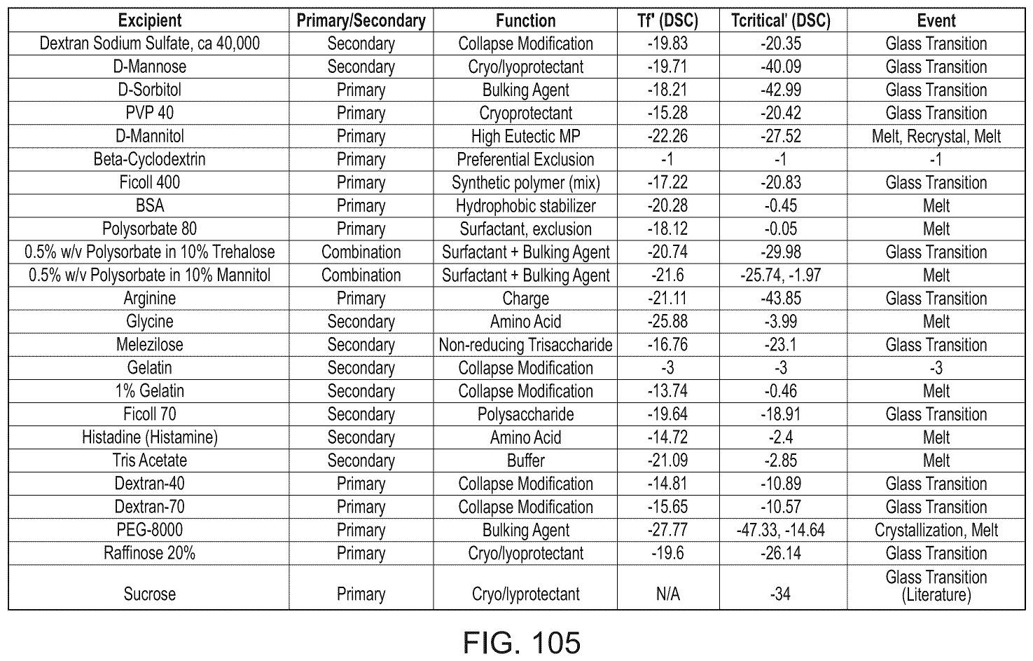

Described herein are various methods and devices for carrying out DETECTR assays. In some embodiments, DETECTR assays utilize the Cas12 variant (SEQ ID NO: 17) protein. In some embodiments, RT-LAMP and DETECTR master mixes of reagents are lyophilized in the same combined master mix. In some embodiments, RT-LAMP and DETECTR master mixes of reagents and target are lyophilized in the same combined master mix. FIG. 99A presents results for a one-pot RT-LAMP assay. In some embodiments, one-pot refers to the combination of both RT-LAMP and DETECTR reaction reagents in one sample. FIG. 99B presents results for a Cas12 variant-based DETECTR assay using SEQ ID NO: 17. The left-hand y-axis plots raw fluorescence. The x-axis plots time in minutes. Subplot 1 presents results where master mixes were not lyophilized and did not contain excipients. Subplot 2 presents results where master mixes were not lyophilized but did contain excipients. In some embodiments, an excipient is used confirm reagent stability throughout the lyophilization process, comprising freezing and drying steps. In some embodiments, excipients are sugars. In some embodiments, excipients comprise one or more of the compounds listed in FIG. 105. Subplots 7 and 11 of FIG. 99A presents results of an embodiment of a master mix containing the excipients Trehalose and Raffinose, wherein Trehalose and Raffinose are included at various percentages as described on the right-hand y-axis. Subplot 7 presents results for a mixture with 10% Trehalose and 3% Raffinose. Subplot 11 exhibits data for a mixture with 10% Trehalose and 5% Raffinose. Subplot 8 exhibits data for a mixture with 10% Raffinose and 3% Trehalose. Subplot 12 exhibits data for a mixture with 10% Raffinose and 5% Trehalose.

In some embodiments, the reagents and target from both the RT-LAMP and DETECTR assays were lyophilized in one sample. FIG. 99B shows results for such an embodiment ran through a Cas12 variant-based DETECTR assay using SEQ ID NO: 17 and can be interpreted as FIG. 99A has been described.

In some embodiments, the sample mixtures include multiple target nucleic acids. In some embodiments, the sample mixtures contain multiple copies of nucleic acid targets. In some embodiments, the 1000 copies (cps) of the nucleic acid target are present in one sample. In the legends on the right-hand sides of FIGS. 99A-99B, “1000 cps” refers to 1000 copies of the target and is represented as a solid line. NTC refers to no-target-control and is represented as a large dashed line. The thin solid lines bounding all data series of FIGS. 99A-99B, 100, 101, 102A-102B and 103 represent bounds of the data set. Additionally, in some embodiments, “mix. iteration” refers to the form of the master mix, wherein “Liquid” refers to master mixes that were not lyophilized, represented as a solid line and “Lyo” refers to master mixes that were lyophilized and is represented as a small dashed line.

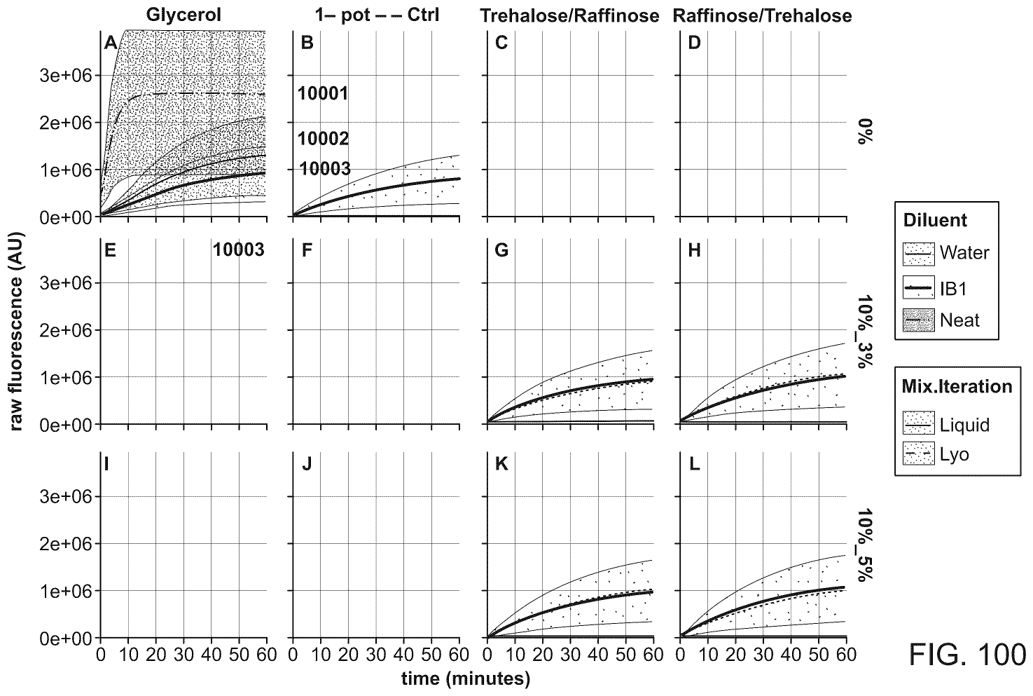

In some embodiments, a master mix of assay reagents are reconstituted after lyophilization. In some embodiments, a master mix of DETECTR assay reagents are reconstituted after lyophilization. In some embodiments, a master mix of DETECTR assay reagents, including a Cas 12 variant (SEQ ID NO: 17) protein are reconstituted after lyophilization. In some embodiments, a master mix of amplification and DETECTR assay reagents, including Cas12 variant (SEQ ID NO: 17) protein are reconstituted after lyophilization. In some embodiments, a master mix of RT-LAMP and DETECTR assay reagents, including Cas12 variant (SEQ ID NO: 17) protein are reconstituted after lyophilization. The results of a reconstituted lyophilized Cas12 variant DETECTR master mix using SEQ ID NO: 17 are shown in FIG. 100. The left-hand, y-axis plots raw fluorescence. The x-axis plots time in minutes. Subplot A contains results for a glycerol control (10001), IB1 buffer (10003) and water (10002), with no excipients present. Subplot B contains results for the one-pot control. Subplot G shows results for a sample containing 10% Trehalose and 3% Raffinose. Subplot K shows results for a sample containing 10% Trehalose and 5% Raffinose. Subplot H shows Raffinose and Trehalsose at 10% and 3%, respectively, and subplot L shows Raffinose and Trehalose at 10% and 5%, respectively. The bold solid trace shows the experimental series where IB1 reaction buffer was used. The thin solid lines, see FIGS. 99A-99B (9901), located on either side of the bold solid line representing the IB1 buffer, show the boundaries of the data.

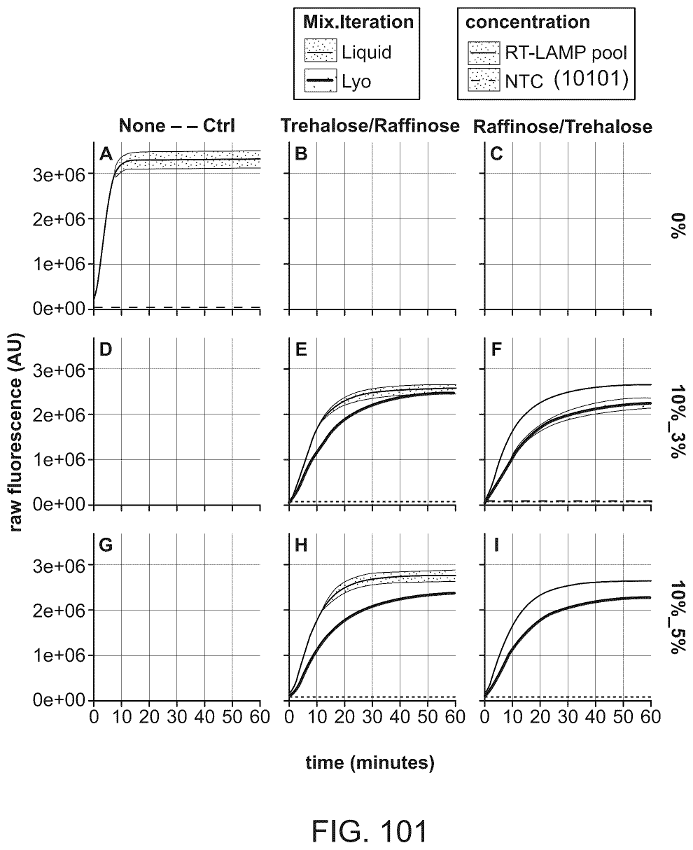

FIG. 101 presents results for a Cas14a1-based DETECTR assay. The left-hand, y-axis plots raw fluorescence. The x-axis plots time in minutes. Subplot A presents results for non-lyophilized control master mix comprising an RT-LAMP pool as the target, seen as the solid line with speckled background for the data boundaries. The thin solid line with speckled background series) contains a non-lyophilized control with no target present. Subplots E, F, H and I present results for master mixes, with RT-LAMP pool as the target. Lyophilized master mixes with RT-LAMP pool as the target are represented by the solid line, while non-lyophilized master mixes are represented by the thin solid line with speckled background series. Lyophilized master mixes without target are represented by the bold line series and non-lyophilized samples without target are represented by the thin solid line with speckled background series. Additionally, subplot E contains excipients 10% Trehalose and 3% Raffinose. Subplot H contains 10% Trehalose and 5% Raffinose. Subplot F contains 10% Raffinose and 3% Trehalose and subplot I contains 10% Raffinose and 5% Trehalose.

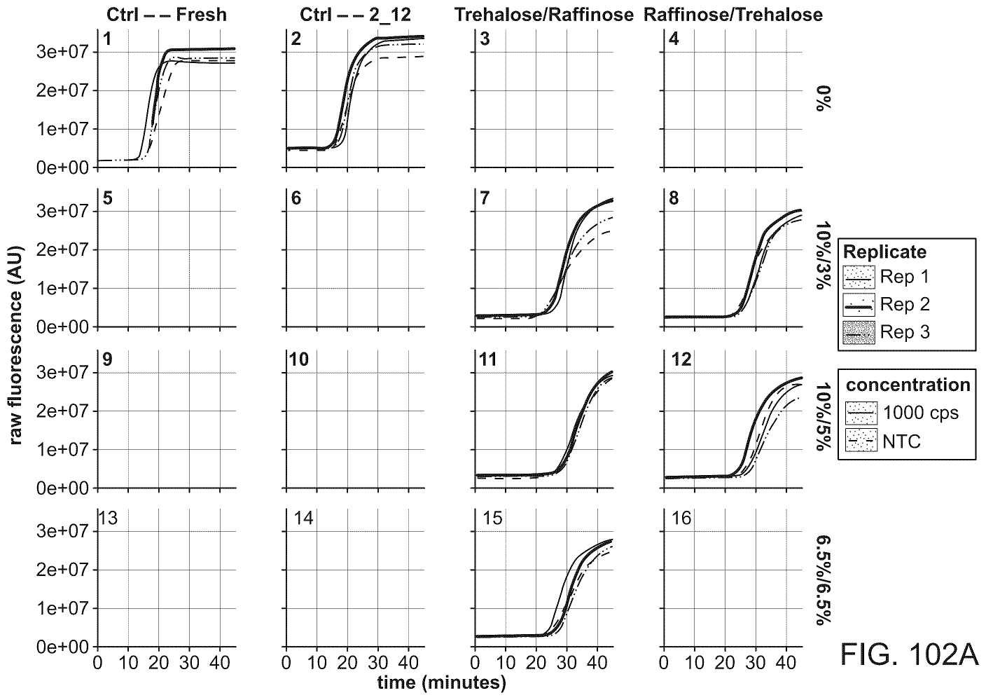

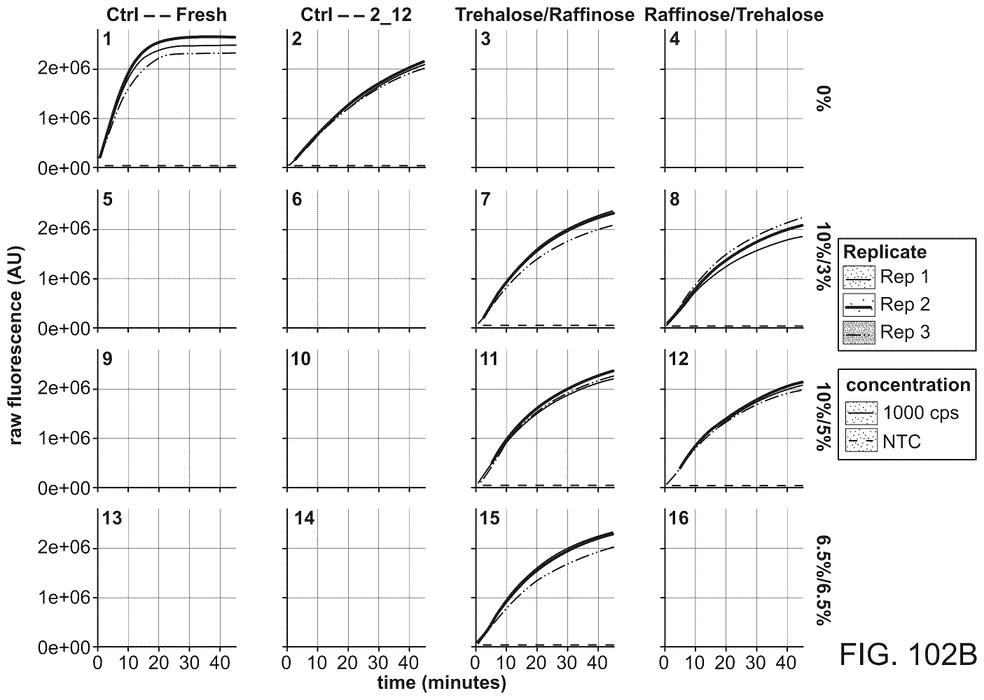

In some embodiments, the master mix of reagents and target for one assay is lyophilized. In some embodiments, the master mixes from more than one assay are pooled and lyophilized. In some embodiments, a duration of time occurs between mixing and lyophilization. FIG. 102A and FIG. 102B present results of an embodiment where the master mixes for both RT-LAMP and Cas12 variant-based DETECTR assays using SEQ ID NO: 17 were each made, mixed together, and stored for two weeks prior to lyophilization. Subplot 1 of FIG. 102A presents results for a sample without excipient and was not lyophilized but was prepared immediately prior to running the RT-LAMP assay. Subplot 2 presents results for a sample that did not contain excipient and was lyophilized two weeks after initial preparation. Subplots 7, 8, 11, 12, 15 and 16 of FIG. 102A show results for samples containing various types and amounts of excipient as described on the axes. Each color line represents one of three replicates. The solid lines represent a concentration of 1000 copies of target per sample and the dashed line contain no targets. FIG. 102B shows similar results for the DETECTR assay ran on an aliquot of the same sample.

In some embodiments, lyophilized master mixes of reagents from more than one assay are prepared in volumes of less than 1 mL. In some embodiments, lyophilized master mixes of reagents from more than one assay are prepared in volumes of less than 250 uL. In some embodiments, lyophilized master mixes of reagents from more than one assay are prepared in volumes of less than 25 uL. In some embodiments, lyophilized master mixes of reagents from more than one assay are prepared in volumes of less than 10 uL. FIG. 103 presents results for lyophilized reactions at 25 uL in volume. Subplots A, B, C show results for an RT-LAMP assay. Subplot A shows RT-LAMP results for lyophilized sample with excipients, where the solid line represents a sample with 500 copies per sample of target and the dashed line represents a no target control. Subplot B presents results for non-lyophilized sample with excipients, where the solid line represents a sample with 500 copies per sample of target and the dashed line represents a no target control. Subplot C presents results for samples without excipients. Subplots D, E, and F show results for a Cas12 variant-based DETECTR assay using SEQ ID NO: 17. Subplots G, H and I show results for a Cas14-based DETECTR assay. For both assays, the solid lines show results for samples with the same targets used in the RT-LAMP assay, with three replicates. The dashed lines represent no target controls.

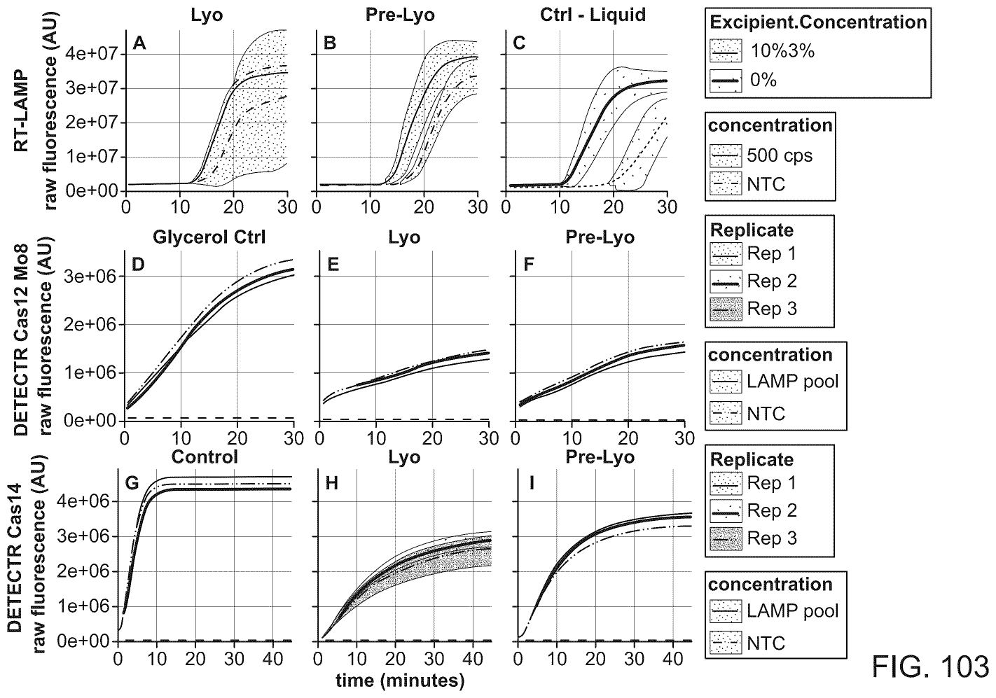

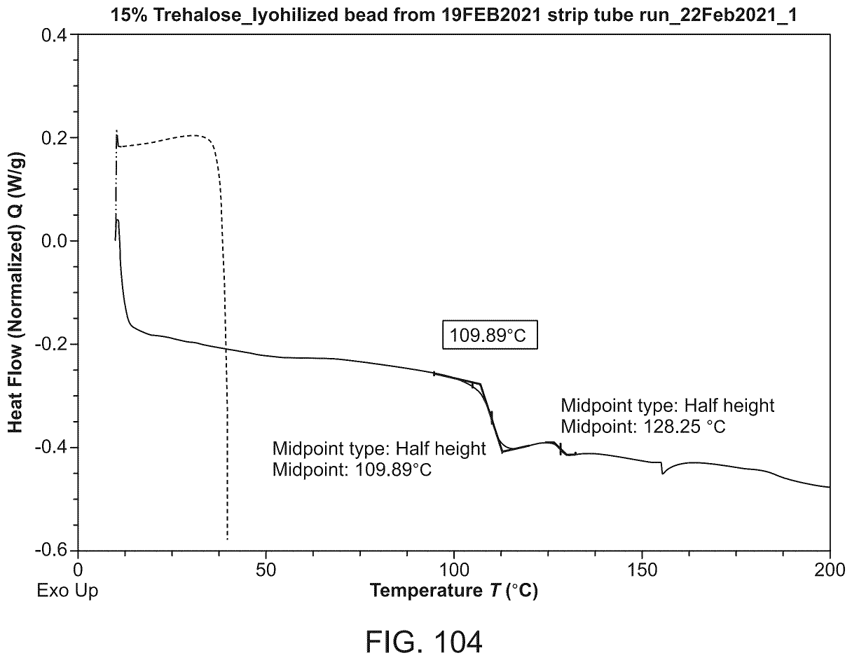

In some embodiments, lyophilized master mixes of assay reagents are analyzed by dynamic scanning calorimetry (DSC). FIG. 104 plots heat flow versus temperature in degrees Celsius for such an embodiment. In some embodiments, such measurements provide a measure of quality of the master mix throughout the lyophilization process, specifically the freeze dry steps. In some embodiments, DSC measurements yield a glass transition temperature which can be indicative of long-term stability of a sample.

In some embodiments, an excipient is used to stabilize the sample throughout the lyophilization process that may comprise freezing and drying steps. FIG. 105 presents a list of excipients as well as each excipient's primary/secondary status, function, freezing temperature (T_{f}) as determined by DSC, critical temperature (T critical) as determined by DSC, and event. In some embodiments, T_{critical} is the temperature required to ensure that the sample is fully frozen.

In some embodiments, the hygroscopicity of enzymes and reagents is optimized to improve lyophilization performance.

LAMP Amplification with Cas14a DETECTR in Single Reaction Volume (One-Pot)

Described herein are various methods of sample amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In some embodiments, sample amplification may comprise LAMP amplification.

FIGS. 54A-54B presents results for an embodiment of HotPot involving LAMP amplification with Cas14a DETECTR in single reaction volume (one-pot) In such an embodiment, Cas14 fails to function as a one-pot in standard LAMP conditions. In some embodiments, Cas14a was tested to see if it can function in a one-pot reaction using LowLAMP at 50 C using Klenow (exo-) as the DNA polymerase. (a) The signal from SYTO9, a DNA binding dye, indicates the production of DNA by LAMP. It was confirmed that LowLAMP was able to generate DNA amplicon in the presence of Cas14 in three different buffers. (b) The signal from the Cas14 FQ reporter in the one-pot reaction condition shown in (a). No signal was detected in the one-pot reaction, even though DNA was generated. For this embodiment, results suggest that Cas 14 is inhibited in the LAMP reaction.

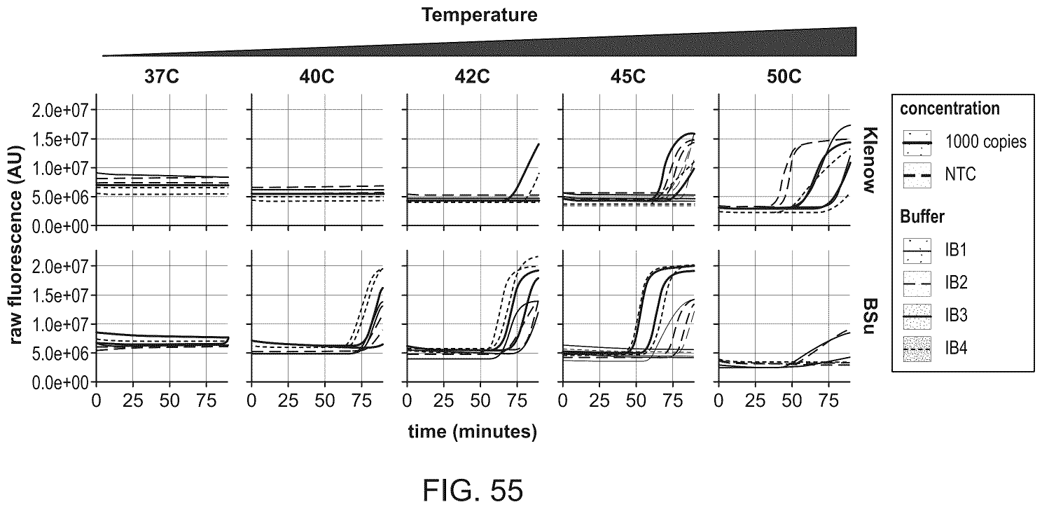

For some embodiments, RT-LAMP can be performed at lower temperatures by using Klenow (exo-) or Bsu polymerases: LowLAMP. RT-LAMP is normally performed at temperatures between 55C-70 C. Results can be seen in FIG. 55. This temperature range is influenced by the polymerase used. In standard RT-LAMP, a Bst or Bsm polymerase is used which show peak activity above 55 C. In this embodiment, the performance of RT-LAMP at temperatures from 37 C to 50 C. By using Klenow (exo-) and Bsu polymerases was evaluated and demonstrated functional RT-LAMP on an RNA target of SARS-CoV-2 at temperatures as low as 40 C. For this embodiment, the performance of these enzymes in 4 different buffers was tested and peak activity for Klenow (exo-) was observed at 50 C, and peak activity for Bsu was at 45 C. For some embodiments, this method is called LowLAMP, as it functions at lower temperatures than standard RT-LAMP.

The Cas14a1 sequence is:

(SEQ ID NO: 3)
MAKNTITKTLKLRIVRPYNSAEVEKIVADEKNNREKIALEKNKDKVKEA
CSKHLKVAAYCTTQVERNACLFCKARKLDDKFYQKLRGQFPDAVFWQEI
SEIFRQLQKQAAEIYNQSLIELYYEIFIKGKGIANASSVEHYLSDVCYT
RAAELFKNAAIASGLRSKIKSNFRLKELKNMKSGLPTTKSDNFPIPLVK
QKGGQYTGFEISNHNSDFIIKIPFGRWQVKKEIDKYRPWEKFDFEQVQK
SPKPISLLLSTQRRKRNKGWSKDEGTEAEIKKVMNGDYQTSYIEVKRGS
KIGEKSAWMLNLSIDVPKIDKGVDPSIIGGIDVGVKSPLVCAINNAFSR
YSISDNDLFHENKKMFARRRILLKKNRHKRAGHGAKNKLKPITILTEKS
ERFRKKLIERWACEIADFFIKNKVGTVQMENLESMKRKEDSYFNIRLRG
FWPYAEMQNKIEFKLKQYGIEIRKVAPNNTSKTCSKCGHLNNYFNFEYR
KKNKFPHFKCEKCNFKENADYNAALNISNPKLKSTKEEP.

In some embodiments, a tracrRNA, known as R1518 is used and is native for the system and has the sequence of:

(SEQ ID NO: 1493)
CUUCACUGAUAAAGUGGAGAACCGCUUCACCAAAAGCUGUCCCUUAGGG
GAUUAGAACUUGAGUGAAGGUGGGCUGCUUGCAUCAGCCUAAUGUCGAG
AAGUGCUUUCUUCGGAAAGUAACCCUCGAAACAAAUUCAUUU.

In some embodiments, Cas14a. 1 uses two RNA components, a tracrRNA and a crRNA. In some embodiments, the native crRNA repeat that occurs with this system is used.

In some embodiments, crRNAs used are:

R3297-SARS-CoV-2 N-gene having the sequence of:
(SEQ ID NO: 1494)
GUUGCAGAACCCGAAUAGACGAAUGAAGGAAUGCAACCCCCCAGCGCUU
CAGCGUUC;
R3298-Mammuthus-having the sequence of:
(SEQ ID NO: 1495)
GUUGCAGAACCCGAAUAGACGAAUGAAGGAAUGCAACGCCGAUAAUGAU
GUAGGGAU;
R4336-SARS-CoV-2-having the sequence of:
(SEQ ID NO: 1496)
GUUGCAGAACCCGAAUAGACGAAUGAAGGAAUGCAACUGGCACUGAGAA
UUUGACUA;
and
R4783-OC43-having the sequence of:
(SEQ ID NO: 1497)
GUUGCAGAACCCGAAUAGACGAAUGAAGGAAUGCAACUACAAGUGCCUG
UAGGUAUA.

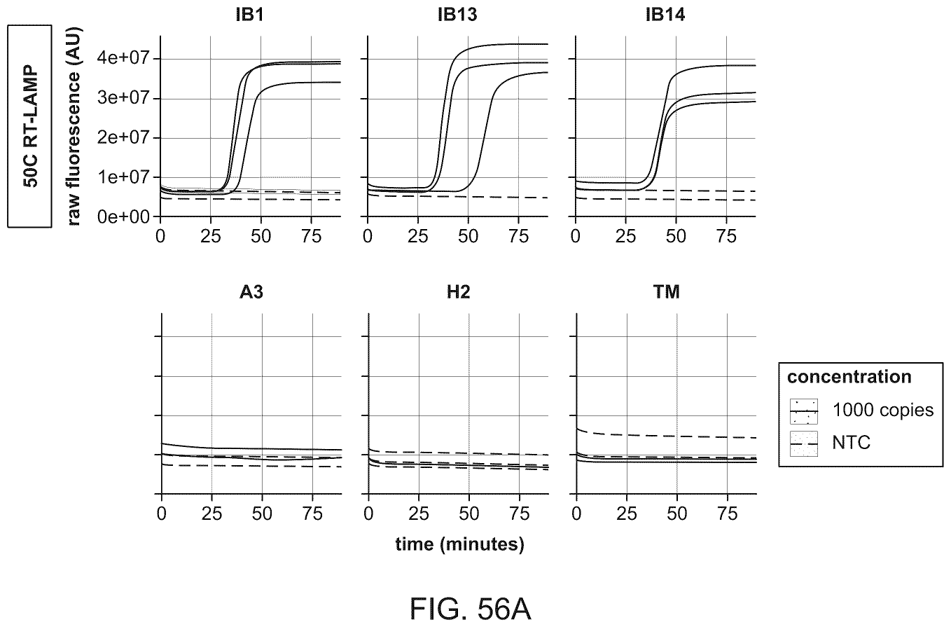

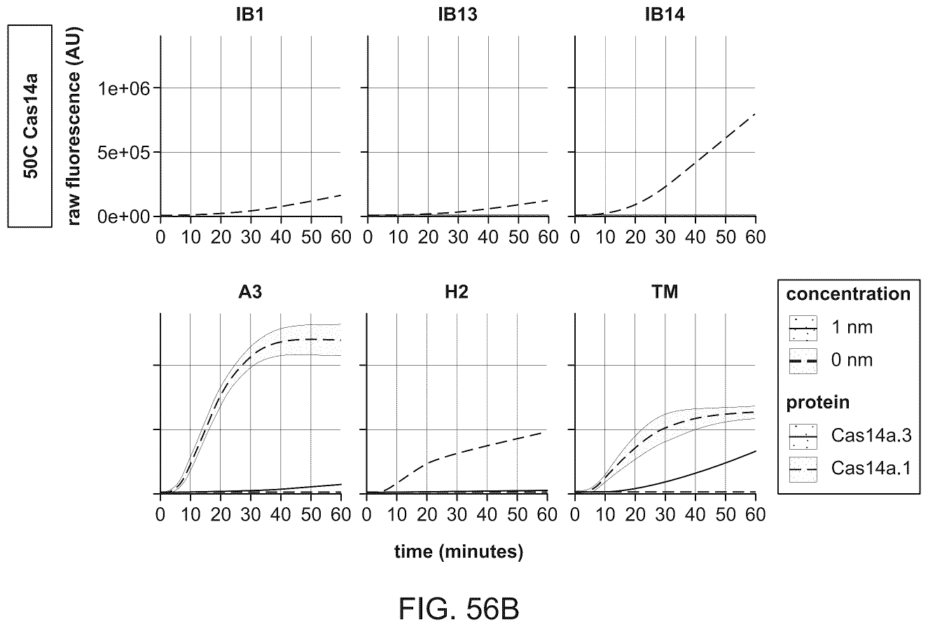

For some embodiments, buffers that were compatible with Cas14a and low temperature RT-LAMP (LowLAMP) were identified. Results are shown in FIGS. 56A-56B. For this embodiment, LowLAMP and Cas14a were found to be optimally functional in different buffers. For this embodiment, the performance of LowLAMP at 50 C in buffers that are optimal for LowLAMP (IB1, IB13, IB14) and buffers that are optimal for Cas14a (A3, H2, TM) was evaluated as well as the performance of Cas14a in the same buffers at 50 C. Results in FIGS. 56A-56B show that both LowLAMP and Cas14a are functional in the LowLAMP optimal buffers, but Cas14a is most compatible with buffer IB14. This work enables the identification of a buffer condition that is compatible with both isothermal amplification and Cas14a DETECTR, a key step in moving towards a one-pot reaction.

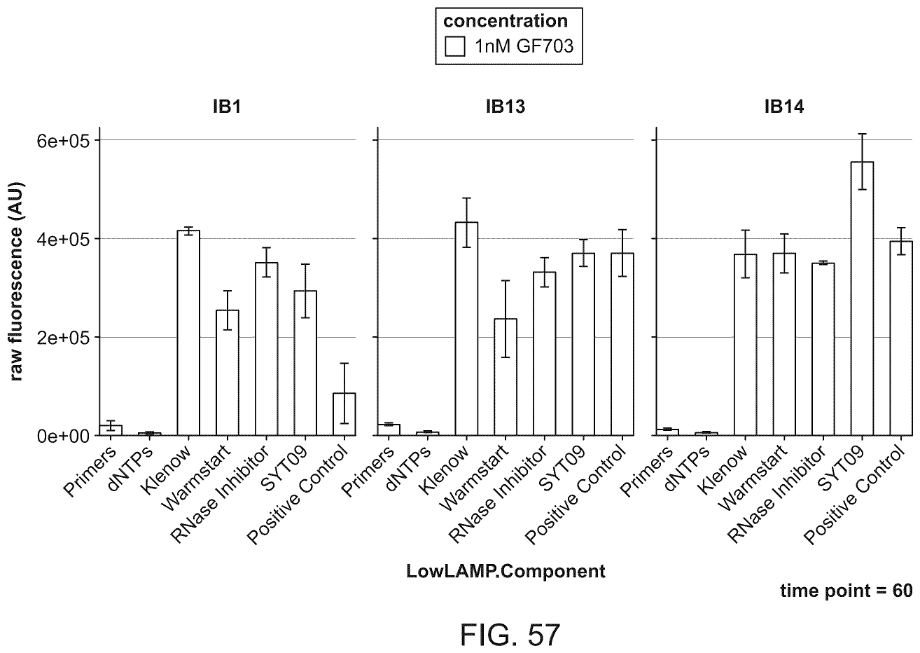

In some embodiment, primers and dNTPs have the greatest inhibitory effect on Cas 14 performance as seen in the results presented in FIG. 57. Here the impact of the individual components of LowLAMP on the performance of Cas14 were tested to determine which components might be responsible for the inhibition seen when attempting one-pot reactions. Each component was added to the Cas14a reaction with 1 nM of target dsDNA and the reaction was run for 60 minutes at 50 C. The results indicate that primers and dNTPs contribute to the inhibition of LAMP.

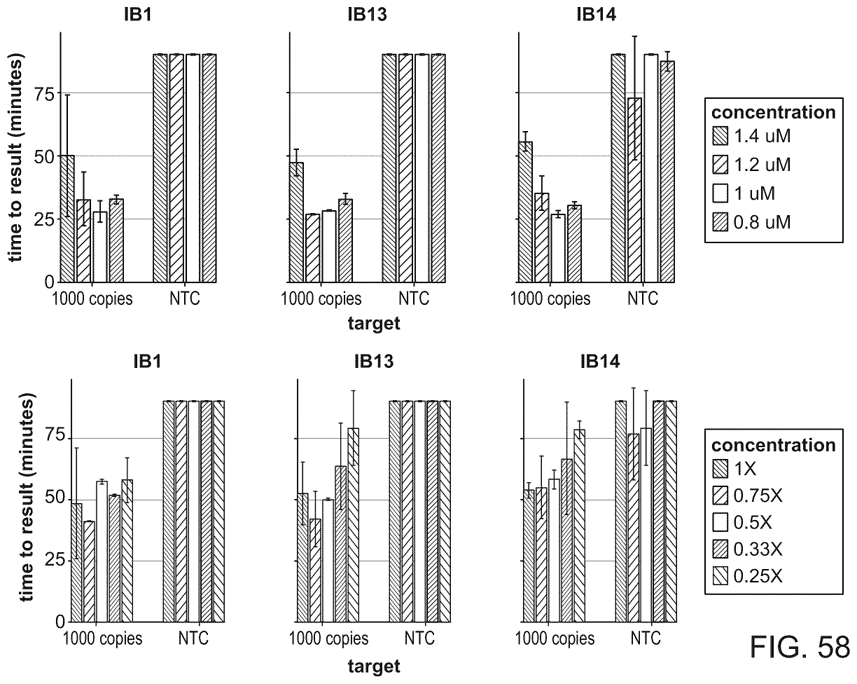

In some embodiments, LAMP functions with lower concentrations of dNTPs and primers as shown in FIG. 58. In some embodiments, both the dNTPs and primers in LAMP have an inhibitory effect on Cas14. In this embodiment, the performance of lower concentrations of dNTPs and primers was tested to see the impact on the performance of LowLAMP using Klenow (exo-) at 50 C. For this embodiment, results show a time-to-result where a lower value indicates faster amplification. (a) Titration of dNTPs shows that LowLAMP functions down to 0.8 mM dNTPs. For this embodiment, the performance of the assay was improved at 1.2 mM and less versus the standard 1.4 mM. (b) Titration of primers in LowLAMP. The results of this assay show that going below 0.5× of primers can have an inhibitory effect. Together these results suggest that the dNTPs and primers concentrations can be reduced without negatively impacting LowLAMP performance, which may help relieve the observed inhibition of Cas14.

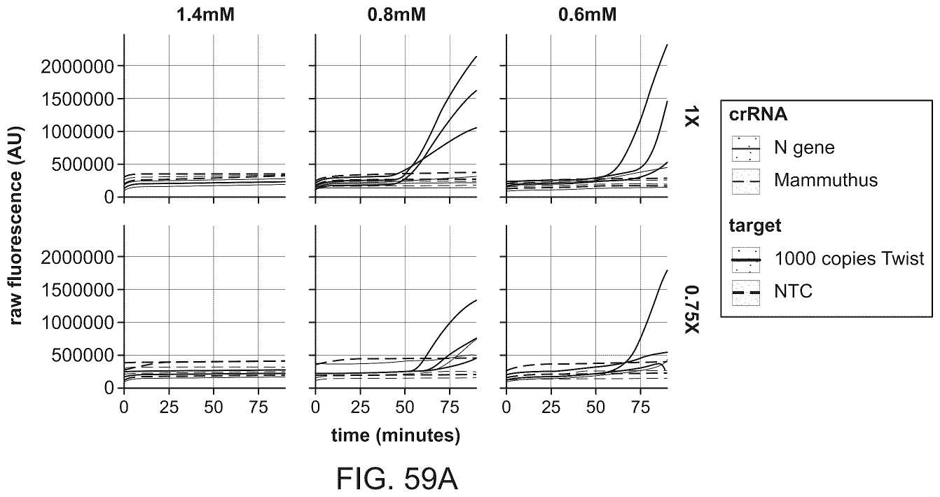

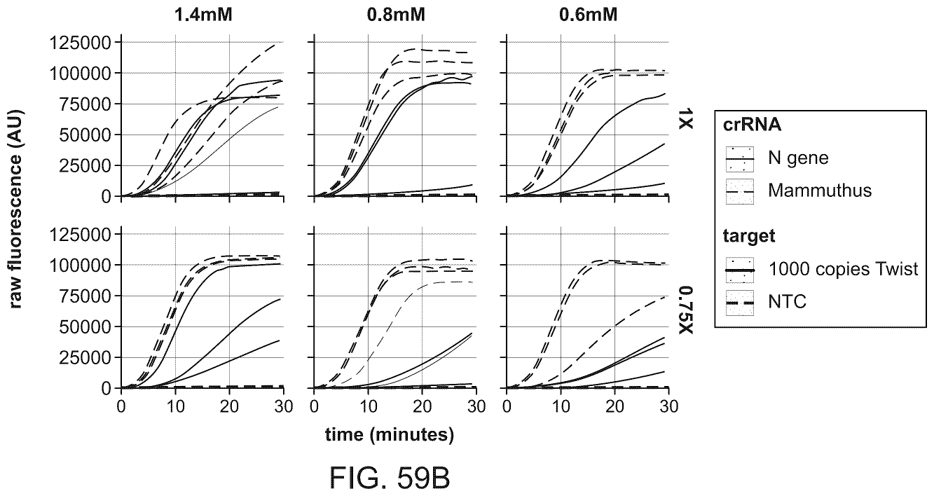

In some embodiments, One-pot Cas14 with LowLAMP was tested at 50 C. For such embodiments, one-pot DETECTR using Cas14 and LowLAMP at 50 C using a Klenow (exo-) DNA polymerase was shown to be functional as seen in FIGS. 59A-59B. For this embodiment, lower primer and dNTP concentrations were tested. Other embodiments have suggested that Cas 14 is inhibited at the standard 1.4 mM and 1× primer concentration. (a) Signal from Cas14a one-pot. For this embodiment, one-pot DETECTR reactions using a Cas14 were either complexed with a SARS-CoV-2 N-gene crRNA that targeted the amplicon generated by LowLAMP, or complexed with a non-target crRNA that targeted a gene in the Mammuthus primigenius mtDNA. Results from this embodiment, showed a signal only in the reaction that were run with target RNA and the crRNA targeting the amplicon. No signal was seen when there was no target present, or the Cas14 crRNA was targeting the Mammuthus gene. (b) To confirm the generation of the target of interest by LAMP, amplicons generated in the one-pot reaction were run in a Cas12 DETECTR reaction using a SARS-CoV-2 N-gene crRNA. These results demonstrated that the amplicon was generated when the target RNA was present. Furthermore, the results indicate that the one-pot reaction is functional at 0.8 and 0.6 mM dNTPS, but not at 1.4 mM dNTPs due to Cas14 inhibition at this concentration of dNTPs.

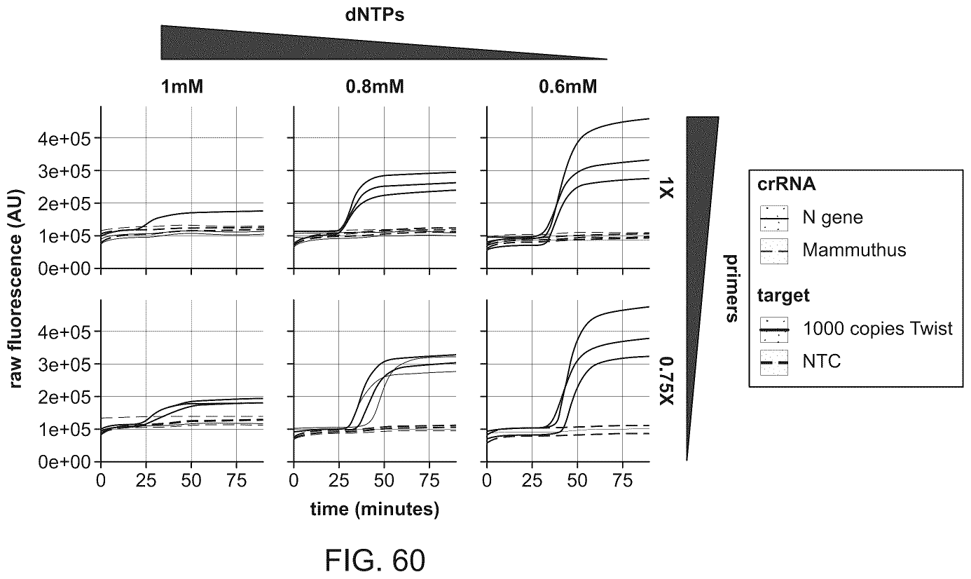

In some embodiments, Cas14 is used with a polymerase (e.g., a Bsm DNA polymerase, a Bst DNA polymerase, a Klenow (exo-) DNA polymerase, or a Bsu DNA polymerase) (55C) for the OnePot assay. The one-pot reaction may be faster if the reaction temperature is increased from 50 C to 55 C. However, the DNA polymerase used in LowLAMP is not functional at 55 C, so here Bsm DNA polymerase was used which works more robustly at 55 C than other LAMP polymerases such as Bst. Several different concentrations of dNTPs and primers were tested and performance of the assay was assessed. Results shown in FIG. 60 indicate that a signal is generated by Cas14 when Cas 14 has the crRNA that matches the target amplicon (N-gene) and when the target RNA is present in the reaction. The speed of the reaction was fastest with 1 mM dNTPs, but the overall signal was lower than when 0.8 and 0.6 mM dNTPs were used.

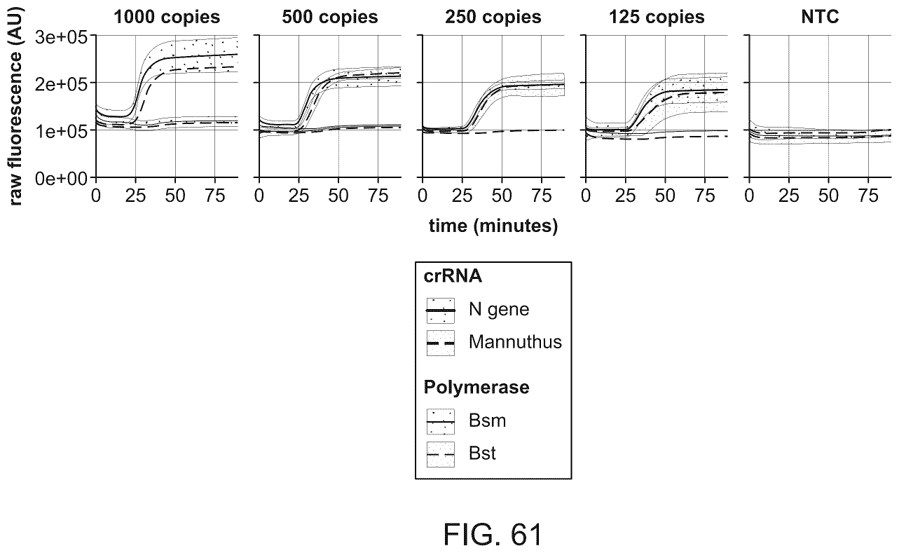

For some embodiments, the initial performance of the one-pot DETECTR reaction, called HotPot was evaluated. Results are shown in FIG. 61. In this experiment, a limit of detection experiment for two different DNA polymerases at 55 C was performed. Cas14 crRNAs targeting either the SARS-CoV-2 N-gene or an off-target gene were included in the experiment. The assay at 1000, 500, 250, or 125 copies/reaction of SARS-CoV-2 RNA genomes was tested. Robust performance of the assay at the lowest tested concentration of 125 copies/reaction for 3/3 replicates as shown in FIG. 61.

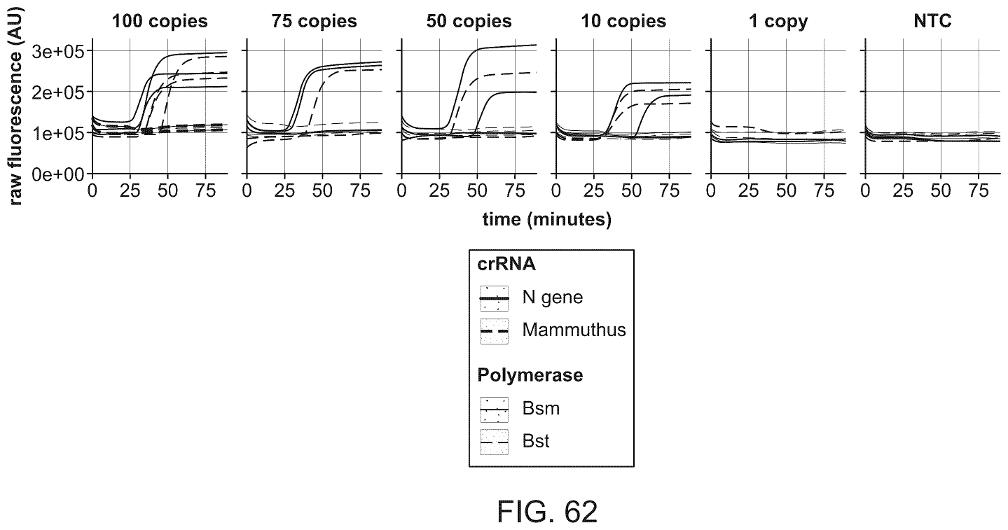

For some embodiments, the limit of detection experiments were performed two different DNA polymerases at 55 C. Results are shown in FIG. 62. For these embodiments, Cas14 crRNAs targeting either the SARS-CoV-2 N-gene or an off-target gene were included in the experiments and the assay at 100, 75, 50, 10, or 1 copies/reaction of SARS-CoV-2 RNA genomes were tested. For these embodiments, robust performance of the assay at 100 copies/reaction for 3/3 replicates was observed. At 75 copies and below several replicates did not show up, but detection down to 10 copies/reaction for ⅔ replicates was observed

For various embodiments described herein, the assay turn-around time is 5 minutes. In some embodiments, the assay turn-around time is 10 minutes. In some embodiments, the turn-around time is 15 minutes. In some embodiments, the turn-around time is 20 minutes. In some embodiments, the turn-around time is 30 minutes. In some embodiments, the turn-around time is 40 minutes or less.

### One-Pot NECTR: NEAR Amplification+Cas14a DETECTR in Single Reaction Volume

Described herein are various methods of sample amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In some embodiments, sample amplification may comprise NEAR amplification.

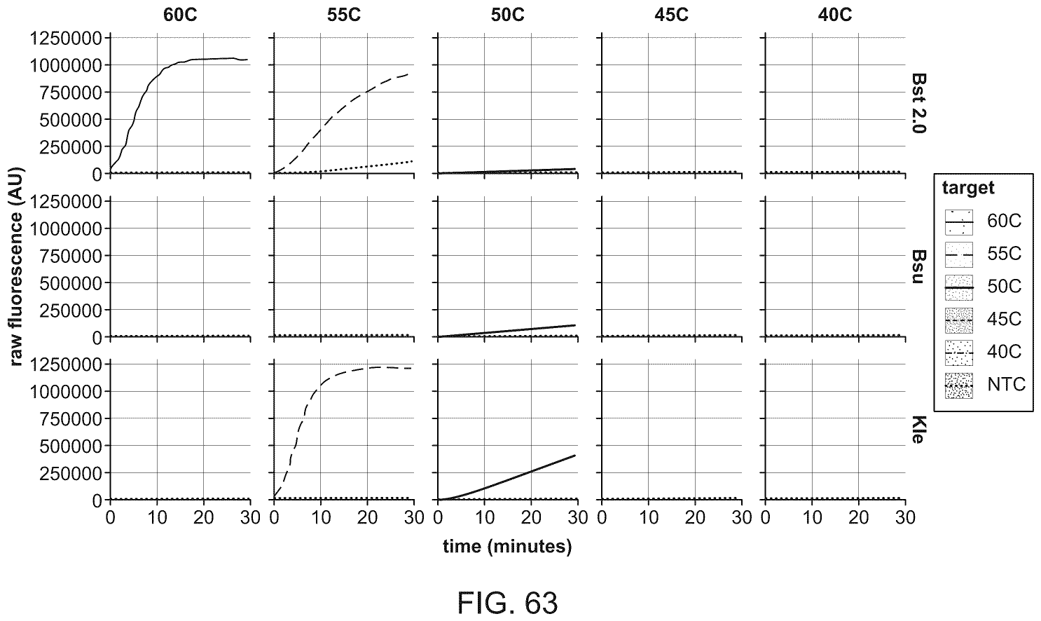

For some embodiments, replacing Bst polymerase in NEAR can enable SARS-CoV-2 detection at lower temperatures as shown in FIG. 63. In these embodiments, NEAR protocol uses Bst 2.0 to generate an amplicon from SARS-CoV-2 at 60 C. This polymerase also functions at 55 C, but at reduced capacity. The performance of Bsu and Klenow (-exo) polymerases at lower temperatures was evaluated for these embodiments. For these embodiments, it was found that Klenow (-exo) polymerase enables robust amplification at 55C and 50 C. Data shown in FIG. 63 is Cas14a DETECTR using SEQ ID NO: 3 following a NEAR amplification reaction. For some embodiments, amplification reactions were performed at the indicated temperature for 10 minutes in a buffer that was composed of a mixture of 1× IsoAmp® buffer+0.5× CutSmart® buffer (NEB). It was observed in this embodiment that Cas14a shows peak activity at 55 C and 50 C, but not at 60 C.

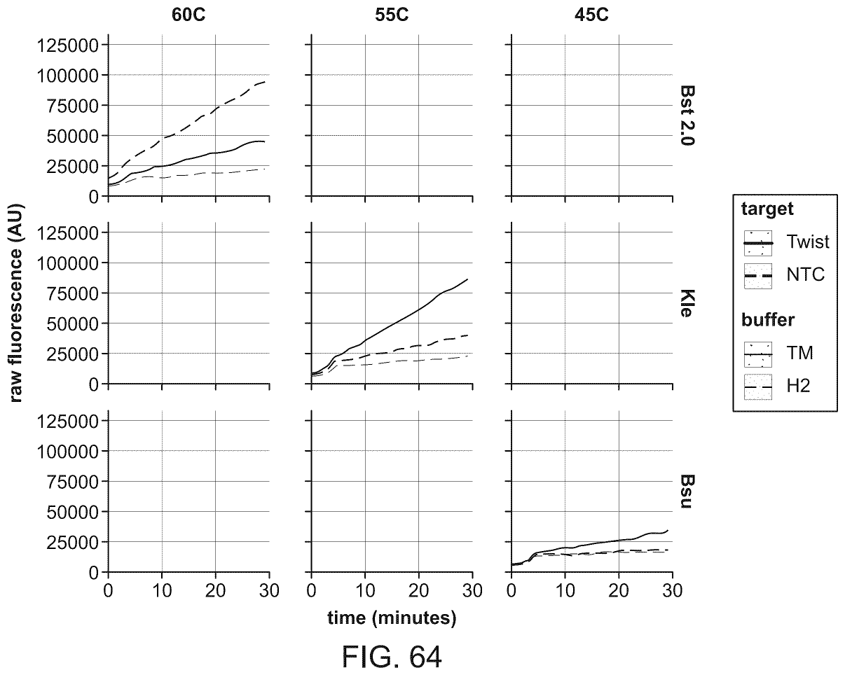

For some embodiments, NEAR amplification functions in Cas14a optimal buffers as shown in FIG. 64. The optimal buffer for Cas14a has less salt and higher Mg2+ than the optimal buffer for NEAR amplification. In this embodiment, the performance of NEAR amplification in two buffers for Cas14a: 1× TM Buffer and 1× H2 Buffer. In this embodiment, amplification reactions were performed with the indicated polymerase, buffer, and reaction temperature with Nt. BstNBI, Omniscript RT, M2805 FWD, M2811 REV. Data shown in FIG. 64 is the result of a Cas14a DETECTR reaction using SEQ ID NO: 3 to evaluate the amount of amplicon produced. For this embodiment, results indicate that TM Buffer and H2 Buffer may function for NEAR amplification. However, results suggest that the reactions are producing ˜10 times less amplicon than an optimal NEAR reaction. This suggests room for further buffer optimization. In some embodiments: 1× TM Buffer: 20 mM Tricine, pH 8.5; 2 mM KOAc; 100 μg/mL BSA; 15 mM MgOAc. In some embodiments: 1× H2 Buffer: 20 mM Tris-HCl, pH 8.8; 2 mM KOAc; 0.1% Tween-20; 17.5 mM MgOAc.

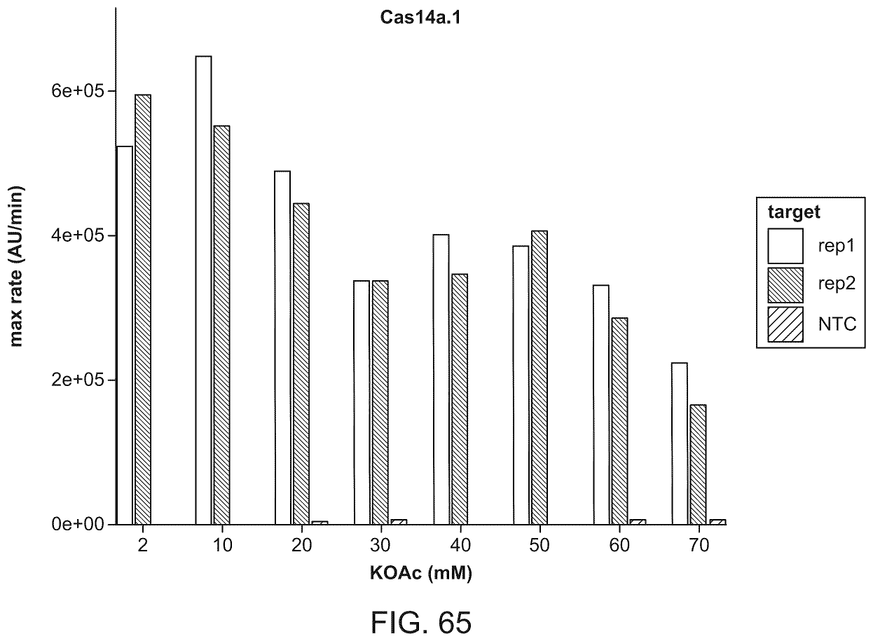

In some embodiments, Cas14a functions in a range of KOAc salt concentrations as shown in FIG. 65. In some embodiments, Cas14a reaction buffer (H2 Buffer) contains 2 mM KOAc. In some embodiments, amplification reactions require higher concentrations for optimal efficiency. In some embodiments, the performance of a Cas14a DETECTR reaction in increasing concentrations of KOAc. Cas14a DETECTR reaction was evaluated with tracrRNA R1518, crRNA R2424, at a target concentration of 1 nM, and a reaction temperature of 50 C. Results shown in FIG. 65 indicate that Cas14a is maximally active at 2-10 mM KOAc, but remains robustly active up to 60 mM. At 70 mM KOAc performance of Cas 14a begins to drop off. In some embodiments, 1× H2 Buffer is composed of: 20 mM Tris-HCl; pH 8.8; 2 mM KOAc; 0.1% Tween-20; and 17.5 mM MgOAc.

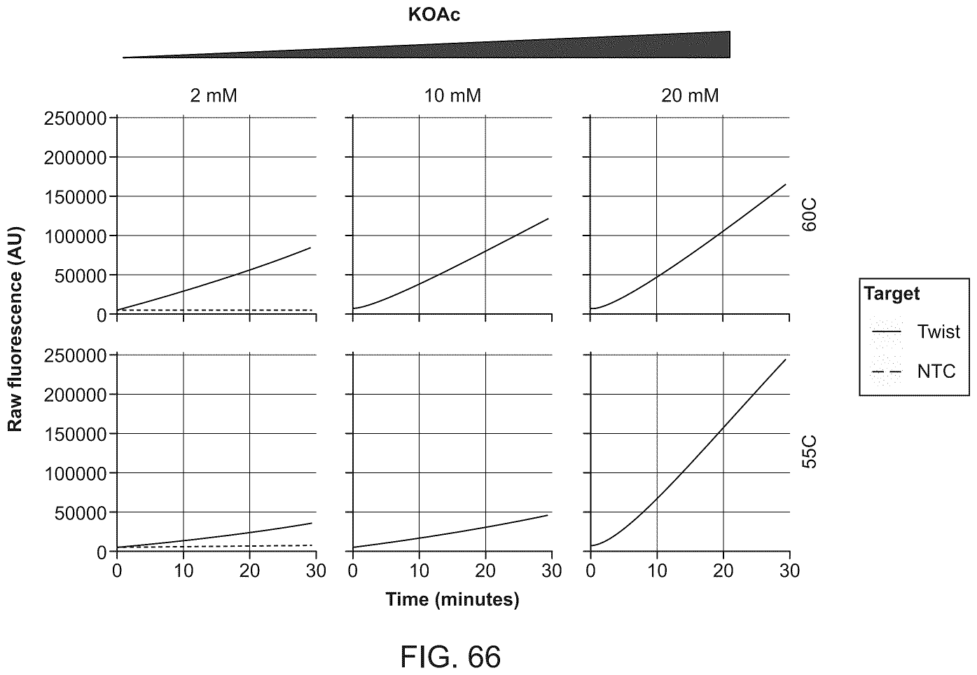

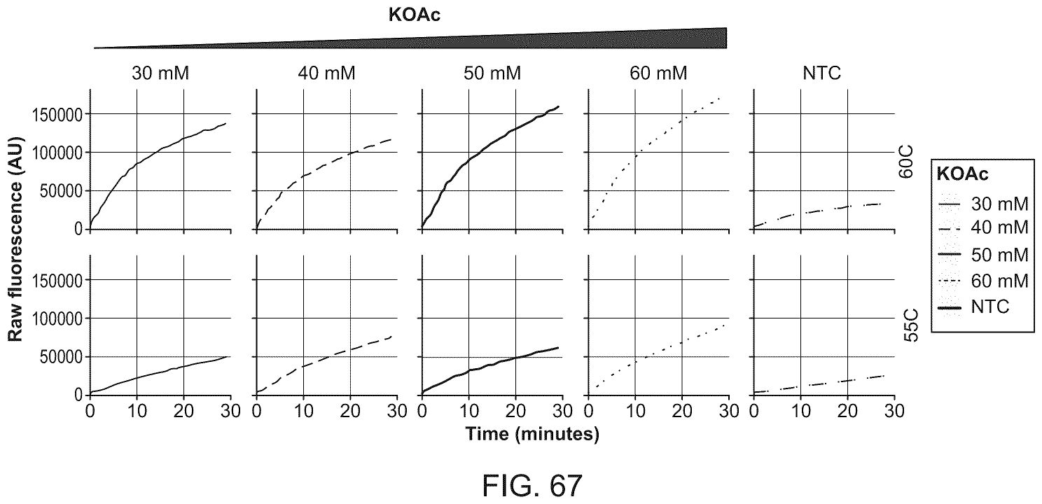

In some embodiments, increasing concentrations of KOAc improve NEAR performance in Cas14a optimal buffers, as seen in FIG. 66. NEAR amplification in Cas14a optimal reaction buffers was initially seen to be less efficient than when amplification is performed in the optimal NEAR buffer (1× IsoAmp®+0.5× CutSmart®). In some embodiments, the impact of increasing amounts of KOAc on the performance of NEAR amplification in the background of a Cas 14a optimal amplification buffer (H2 Buffer). As seen in FIG. 66 results show how Cas12 variant DETECTR assay using SEQ ID NO: 17 performed after NEAR amplification to measure amplification efficiency. Results indicate that increasing KOAc to up to 60 mM improves the amount of NEAR amplicon generated. From 2 mM to 60 mM KOAc are functional ranges for Cas14a. In some embodiments: 1× H2 Buffer: 20 mM Tris-HCl, pH 8.8; 2 mM KOAc; 0.1% Tween-20; 17.5 mM MgOAc. In some embodiments, increasing concentrations of KOAc improve NEAR performance in Cas14a optimal buffers as seen in FIG. 67.

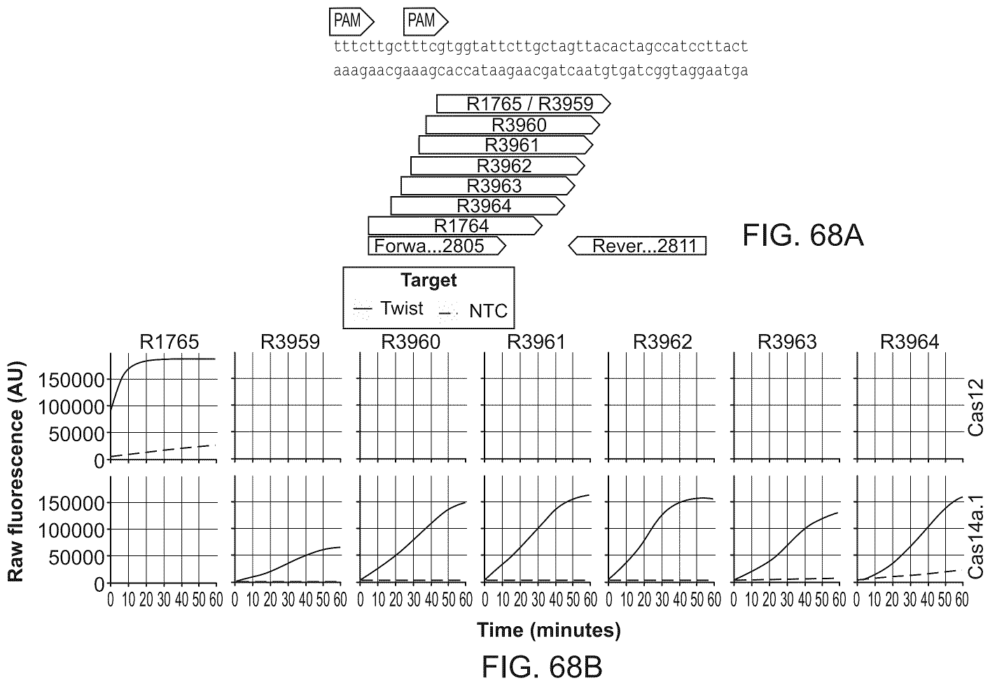

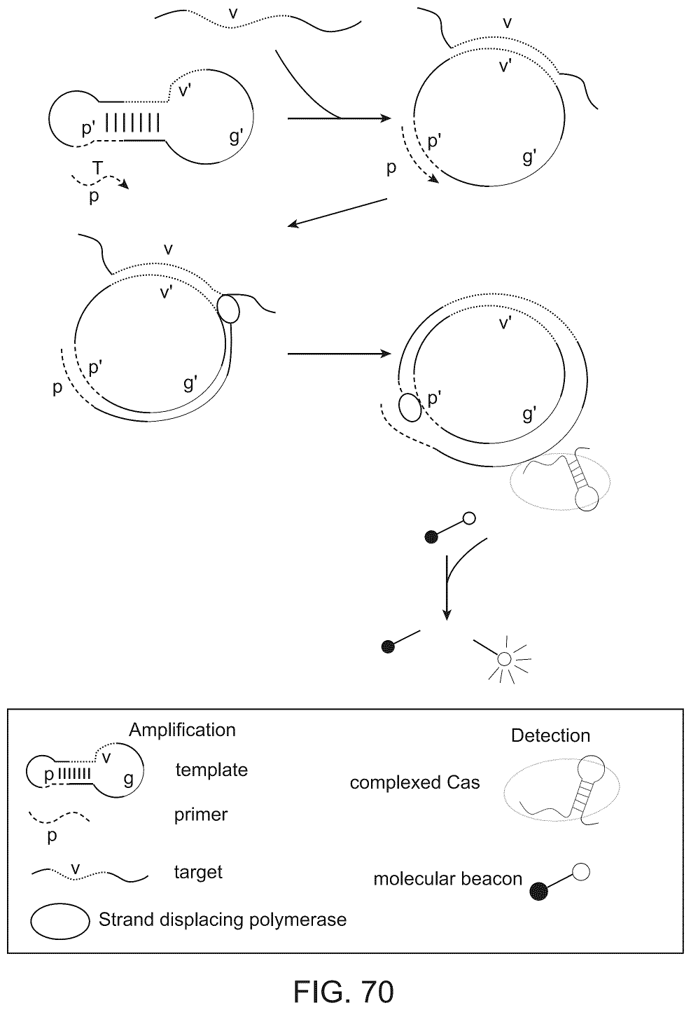

Performance of Cas14a.1 crRNAs on SARS-CoV-2 E-gene amplicon are shown in FIG. 70 for some embodiments. In some embodiments, Cas14a crRNAs, designed to target the SARS-CoV-2 E-gene NEAR amplicon generated by primers M2805/M2811. Results are shown in FIGS. 68A-68B. FIG. 68A shows the position of the crRNAs. R1765 and R1764 are Cas12 variant (SEQ ID NO: 17) crRNAs that target the amplicon. PAM sequences for a Cas12 variant (SEQ ID NO: 17) are indicated, but there are no TTTR PAMs for Cas14 to use within this amplicon. FIG. 68B shows the performance of Cas14a. 1 crRNAs on NEAR amplicon (generated at 60 C for 15 minutes). All tested crRNAs worked robustly with the least amount of background from R3960, R3961, and R3962.

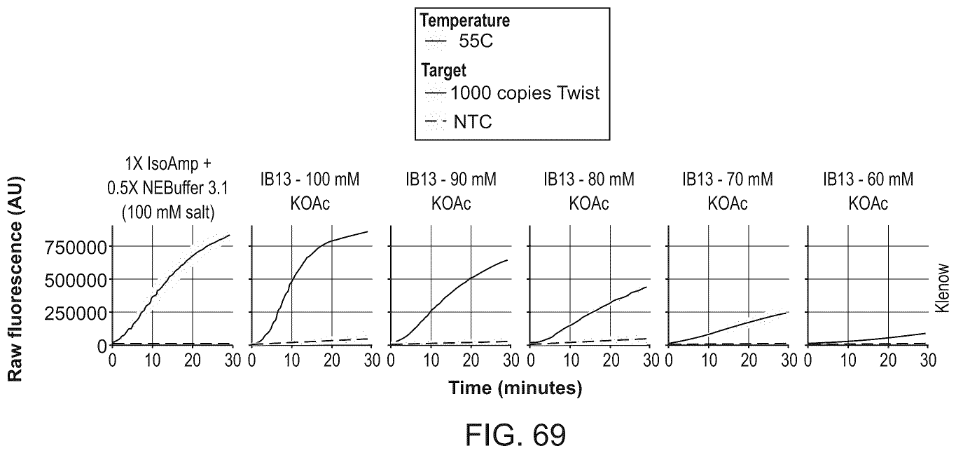

In some embodiments, the performance of Klenow (exo-) NEAR assay in IB13 buffer at decreasing salt concentrations was evaluated as shown in FIG. 69. In these embodiments, standard NEAR reaction buffer was composed a mixture of 1× IsoAmp® buffer (NEB) and 0.5×NEBuffer™ 3.1 (NEB), which gives a final salt concentration of 100 mM. In these embodiments, variations of the IB13 buffer with various concentrations of KOAc as the salt were tested. In these buffer variations the NEAR assay using Klenow (exo-) as the polymerase was run at 55 C for 20 minutes. To readout the amount of amplicon produced, a Cas14a DETECTR assay using SEQ ID NO: 3 on 2 μL of the NEAR amplicon was performed. The results indicate that the best NEAR performance was with 100 mM KOAc, similar to the standard NEAR reaction buffer, and that the amount of amplicon produced was reduced as the salt is reduced. The assay has acceptable performance at 80-70 mM KOAc. In some embodiments, the IB13 buffer has a composition of: 20 mM Tris-HCl, pH 8.8; 10 mM (NH4) 2SO4; 50 mM KOAc; 5 mM MgOAc; 1% Tween-20; 1 mg/mL BSA.

One-Pot sRCA: Rolling Circle Amplification with Cas14a DETECTR in Single Reaction Volume

Described herein are various methods of sample amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In some embodiments, sample amplification may comprise RCA.

FIG. 70 presents an overview of sRCA. In this system, a target nucleic acid is added to a system that contains components for a one-pot RCA+Cas protein reaction. The RCA portion of the system is composed of a dumbbell-shaped DNA template, a primer, and a DNA polymerase. For some embodiments, the DETECTR portion of the reaction is composed of a Cas protein, such as Cas12 or Cas14, a crRNA that targets the amplicon generated by RCA, but not the dumbbell-shaped DNA template, and a FQ ssDNA reporter. A target nucleic acid (e.g., viral RNA), which is capable of binding to the dumbbell DNA template, is added to the system. The target nucleic acid base pairs with the DNA template. The more extensive base pairing between the target and the DNA template causes the internal base pairing of the dumbbell to be disrupted, which opens up a binding site for the primer. The DNA polymerase can then use this primer to begin RCA. In some embodiments, As RCA proceeds, the amplicon is generated which contains the target site for the Cas protein. The Cas protein recognizes this site through base pairing with the crRNA and initiates trans-cleavage of the FQ ssDNA reporter. In some embodiments, the system contains fewer components than other one-pot approaches and does not require a RT enzyme.

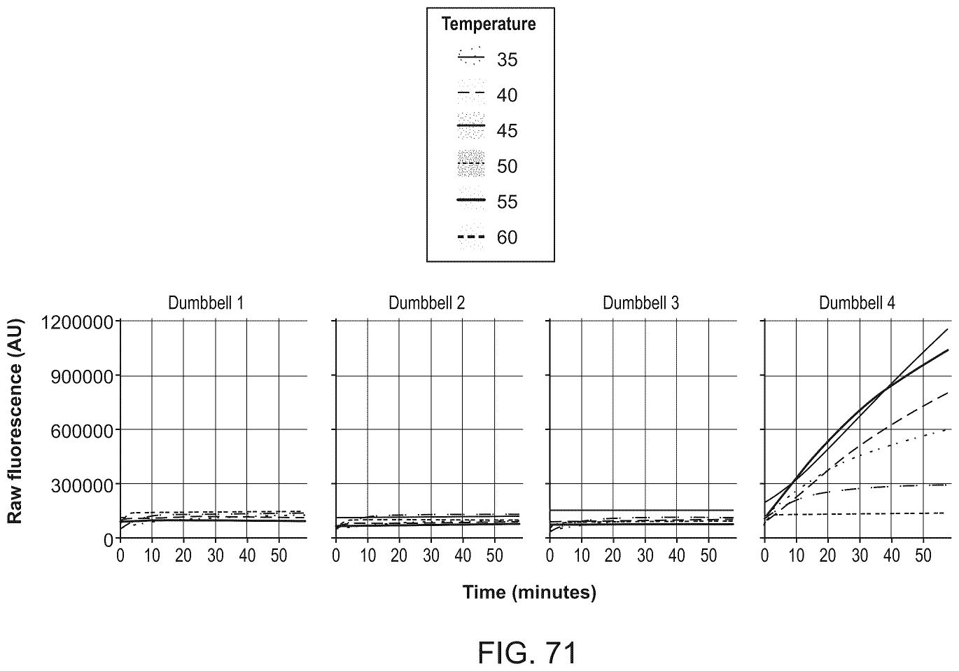

In some embodiments, screening dumbbell DNA templates are screened for sRCA performance, as shown in FIG. 71. In some embodiments, four dumbbell DNA templates for RCA contain a Cas12 or Cas14 target sites. For these embodiments, a DNA binding dye, SYTO9, is used to monitor whether these dumbbells are functional in RCA at a variety of temperatures for 1 hour. Results as seen in FIG. 71 indicate that only dumbbell 4 was functional in generating DNA by RCA. The peak performance of the system was seen at 35-45 C.

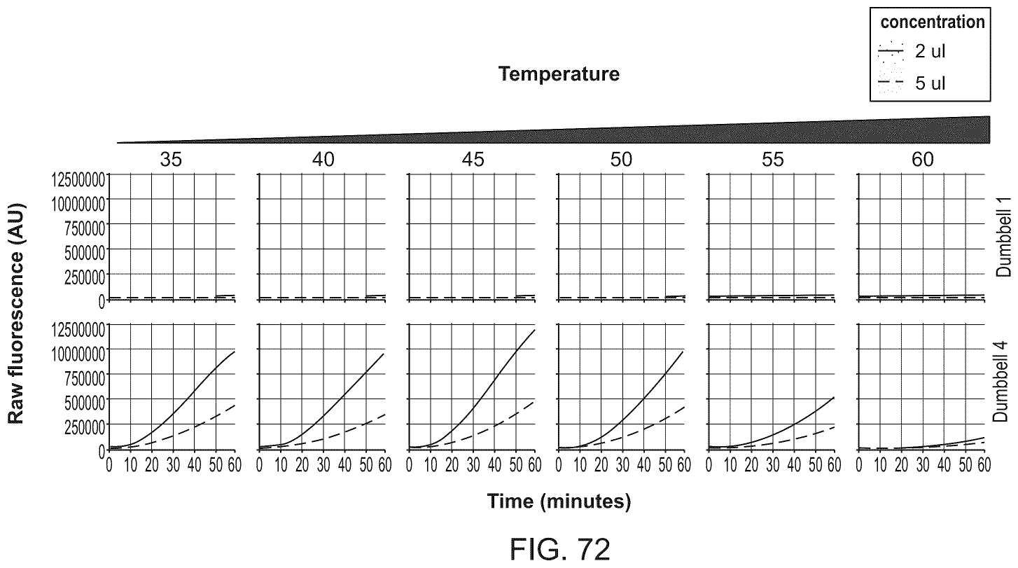

For some embodiments, the performance of Cas14a to detect product of RCA reaction was monitored, as seen in FIG. 72. In other embodiments, it was shown that Dumbbell 4 was functional in RCA and that Dumbbell 1 was not functional. In some embodiments, either 2 μL or 5 μL of the RCA reactions were added to a 20 μL Cas14a DETECTR reaction that contained a crRNA that is capable of detecting the amplicon generated by RCA, but not the Dumbbell DNA template. The results indicate that Cas14a is capable of detecting the amplicon as expected, and that increased performance is seen with lower amounts of RCA added to the reaction.

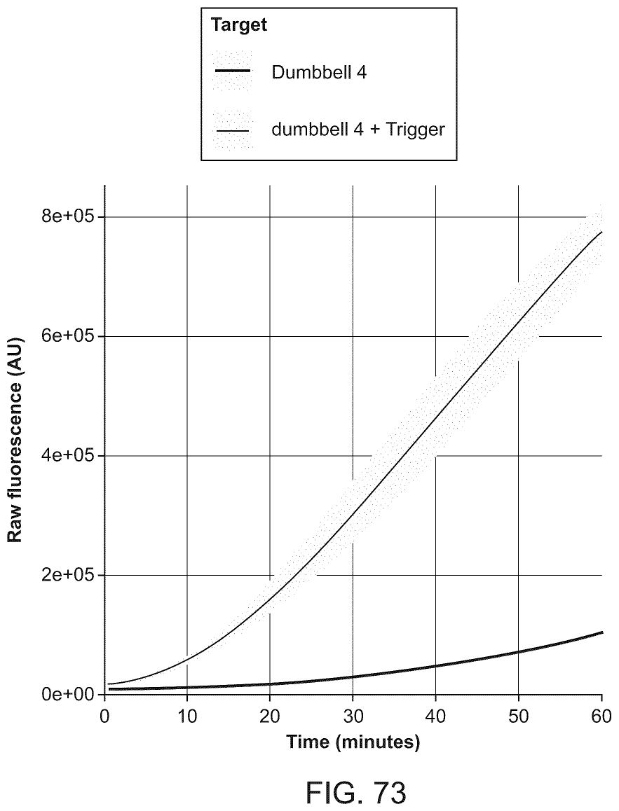

In some embodiments, of the One-Pot assay sRCA, Cas14 is used. Functional results for such embodiments are shown in FIG. 73. The reaction was performed at 45 C in two conditions as two embodiments. In one embodiment, the DNA template, Dumbbell 4, does not contain a trigger oligo and in the other embodiment, the DNA template does have a trigger oligo. In the embodiment with the trigger oligo, which initiates RCA, higher signal is observed as compared to the other embodiment without the trigger oligo. This demonstrates that Cas14 is capable of detecting the RCA amplicon in a one-pot reaction, and that the sRCA reaction is controlled by the presence of the trigger oligo.

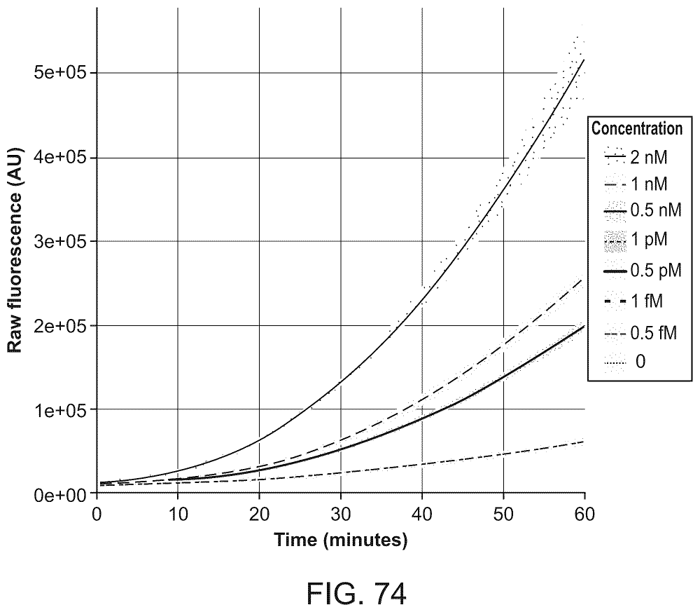

In some embodiments, a trigger oligo is titrated for a Cas14 One-Pot sRCA assay. For this embodiment, the minimal concentration of trigger oligo that is required to initiate the one-pot Cas14 sRCA reaction was determined. Results shown in FIG. 74 indicate that at least 0.5 nM of trigger oligo is required to initiate the sRCA reaction.

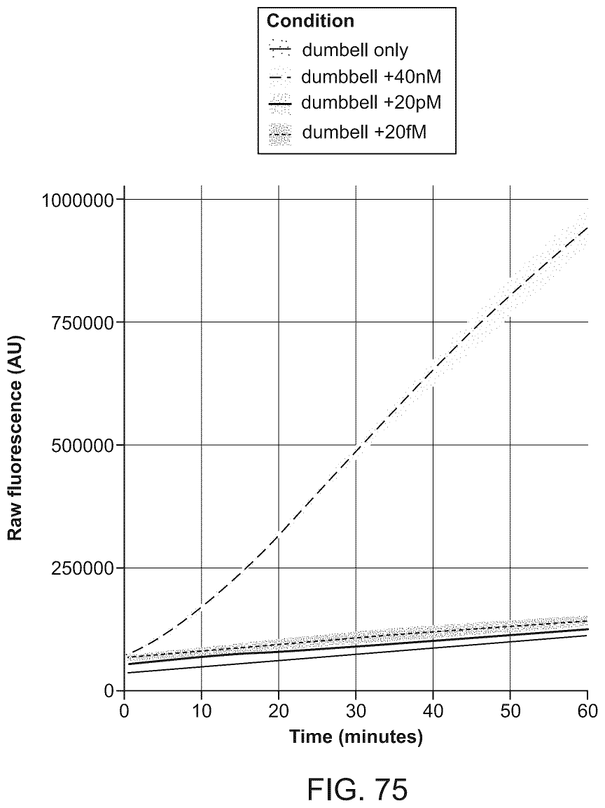

In some embodiments, the Cas 12 variant enzyme (SEQ ID NO: 17) is used in the one-pot sRCA assay. In other embodiments, it has been shown that Cas14 is capable of functioning in a one-pot sRCA reaction. In this embodiment, it was shown that Cas12 variant (SEQ ID NO: 17) is also capable of functioning in this assay at 45 C. The results from the cleavage of ssDNA FQ reporter included in the sRCA reaction are shown in FIG. 75. The results for such an embodiment indicate that Cas12 variant (SEQ ID NO: 17) is capable of functioning in the one-pot sRCA format. In some embodiments, the concentration of stock trigger oligo is: (1) 40 nM stock=2 nM final conc; (2) 20 μM stock=1 μM final conc.; and (3) 20 fm stock=1 fM final conc.

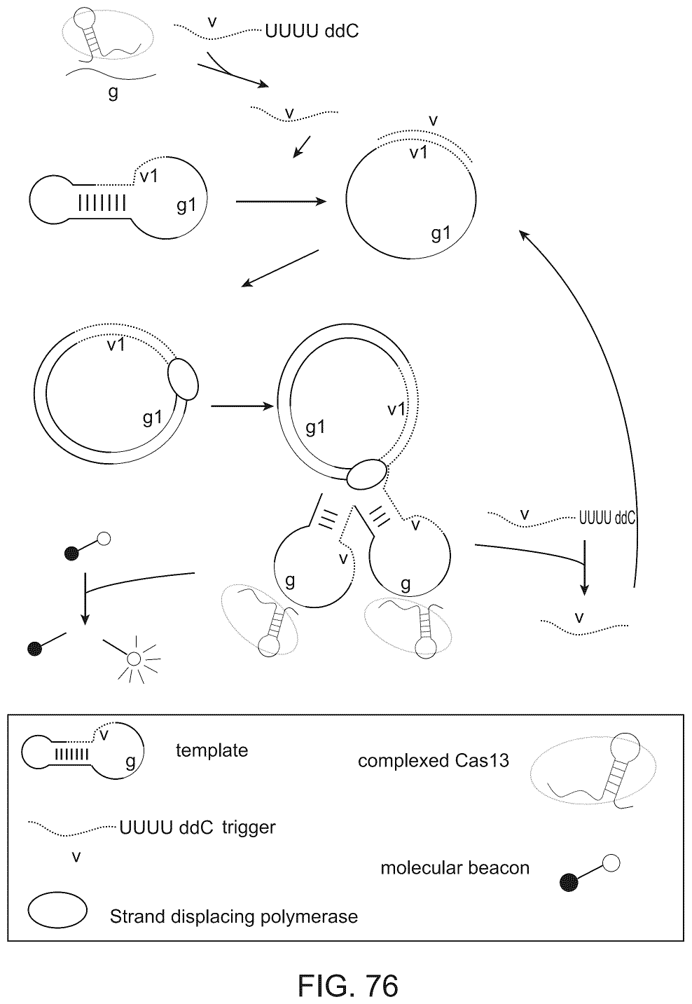

FIG. 76 presents an overview of RCA positive feedback for Cas13. In some embodiments, Cas13 is programmed to recognize an RNA target (g). When the viral RNA target is present, a blocking motif on the 3′ end of the primer (v) is removed. After removal of this blocking motif, the primer can then serve to open up the circular template and allow for amplification by RCA using a DNA polymerase. As the amplicon is generated the same target sequence as the original RNA (g) is generated. This ssDNA target sequence is then capable of being recognized by Cas13 which can either remove additional blocking groups from the primer (v) or cleave a FQ reporter that generates a fluorescent signal. This system forms a positive feedback loop for Cas13.

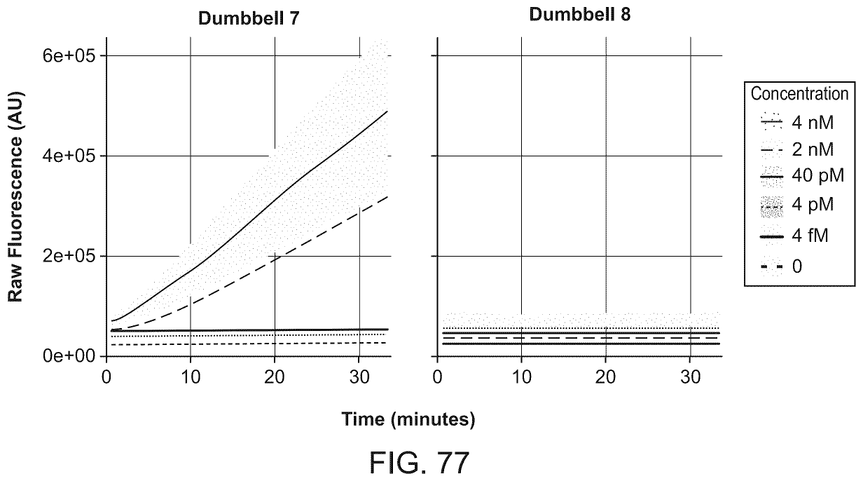

FIG. 77 presents results for the evaluation of Cas13-compatible DNA templates for RCA. In some embodiments, two dumbbell DNA templates for RCA that contain a Cas13 ssDNA targeting site. In some embodiments, a DNA binding dye, SYTO9, is used to monitor whether these two dumbbells are functional in RCA at 30 C. The performance of the two templates was monitored a titration of the primers that are used to trigger the amplification. The results indicate that Dumbbell 7, but not Dumbbell 8 is compatible with RCA as shown in FIG. 77.

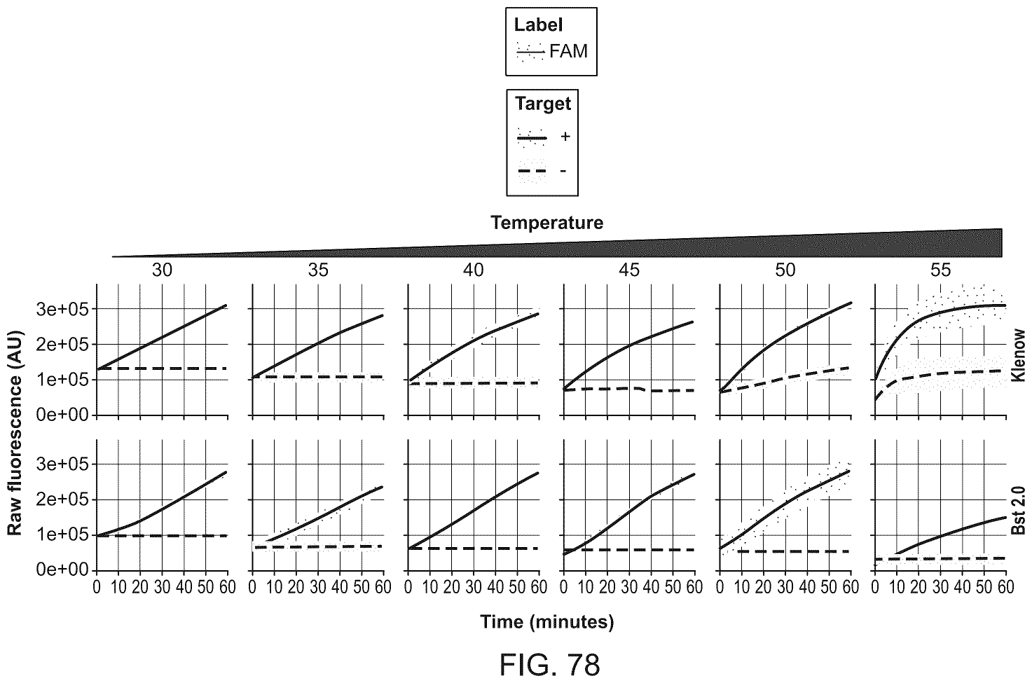

In some embodiments, Cas13-compatible DNA template is used for RCA. FIG. 78 presents results for such embodiments, to determine whether Cas13-compatible DNA template is functional in RCA. In some embodiments a circular DNA dumbbell for sRCA has a Cas13 ssDNA target site, known as dumbbell 7. In some embodiments, a DNA binding dye, SYTO9, is used to monitor whether the DNA template is functional in RCA at various temperatures for two different polymerases using 2 nM of trigger oligo (the primer for sRCA). The results shown in FIG. 78 indicate that dumbbell 7 is capable of generating amplicon at temperatures from 30 to 55 C. Such an embodiment, consisting of the utilization of dumbbell 7 for generating amplicons from 30 to 55 C, enables for the use of the Cas13 enzyme in the OnePot reaction.

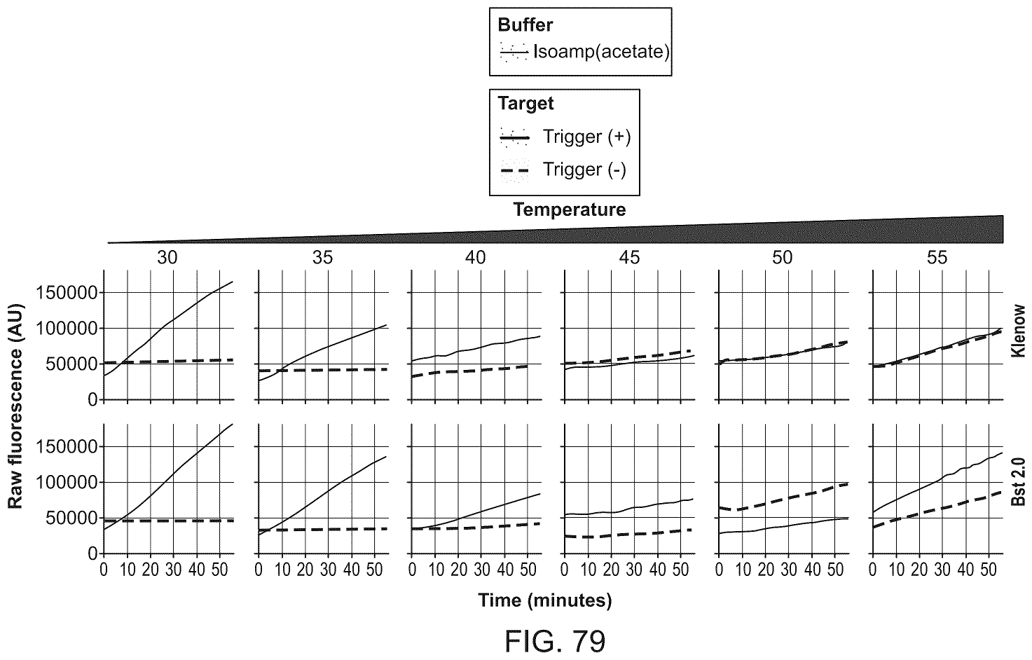

In some embodiments, the Cas 13M26 is used in the one-pot sRCA reaction. FIG. 79 presents results for such an embodiment, where the amplicon generated by RCA contains a ssDNA region that is capable of being recognized by a Cas13 gRNA. As the reaction proceeds, additional ssDNA target is produced, while the activated the Cas13M26 cleaves a FQ RNA reporter to generate a signal. The performance of this reaction at a variety of temperatures was evaluated and it was shown that Cas13M26 is capable of detecting this ssDNA region of the RCA amplicon from 30-40 C, which aligns with the previously established active temperatures for Cas13M26. The performance of two different polymerases at the temperatures of interest was also compared. The results shown in FIG. 79 suggest that Cas13 can be used in a one-pot reaction where RCA is the amplification method, and that Cas13's ability to detect ssDNA is preserved in this embodiment.

CasPin: Cas13 Positive-Feedback Loop Leveraging Cas13 ssDNA Targeting

Described herein are various methods of signal amplification. Any of the devices described herein may be configured to perform signal amplification after the reporter has been cleaved by the programmable nuclease. Signal amplification may improve detection of rare targets in a complex sample. In certain instances, methods include leveraging ssDNA targeting of the programmable nuclease (e.g., Cas13) to create a positive feedback loop upon biding of the programmable nuclease to the target nucleic acid to cleave additional reporters and amplify the signal generated by the presence of the target nucleic acid.

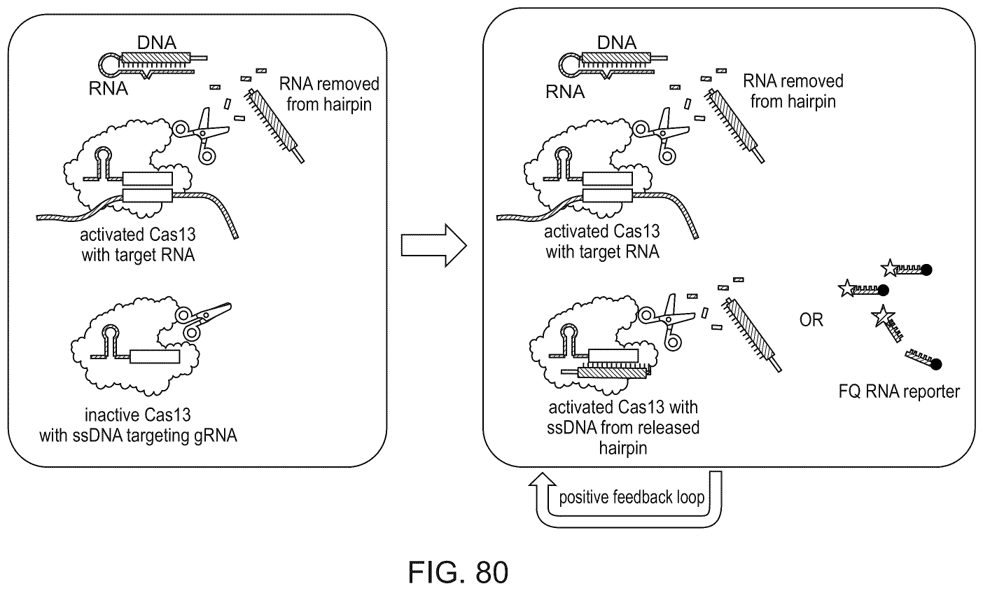

FIG. 80 presents an overview of CasPin. In some embodiments, the CasPin system uses two populations of Cas13. One is programmed with a crRNA that targets an RNA of interest, such as a viral genome. The other population is programmed with a crRNA that is optimal for ssDNA detection. In some embodiments, the systems also contains a hairpin-shaped oligo that is composed of both DNA and RNA. Finally, in some embodiments, there is a FQ RNA reporter that is used to readout the result of the assay. When Cas13 detects the RNA of interest, it can either cleave the FQ RNA reporter or the RNA on the hairpin oligo. When the RNA on the hairpin oligo is cleaved, it dissociates from the DNA revealing a ssDNA target site that can be recognized by the other population of Cas13 RNPs. This initiates a positive feedback loop where Cas13 recognizes the ssDNA target and cleaves more hairpin molecules, which increases the overall amount of target in the system, and leads to further activation of the system. As this process continues more and more FQ RNA reporter is cleaved, which is the ultimate readout of the assay.

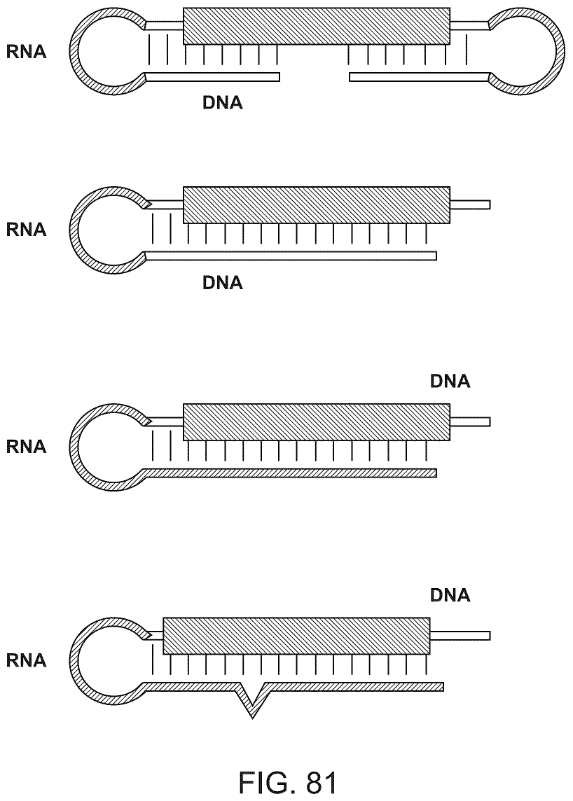

FIG. 81 presents potential structures of hairpins for CasPin. In some embodiments, the target ssDNA sequence is indicated by the purple rectangle. RNA loop structures could occur on either side of the target strand (either 5′,3′ or both). The strand that is complementary to the target site could be DNA or RNA. The strand that is complementary to the target site could also be a perfect match to the target site, be shorter than the target site, or contain mismatches to help destabilize or promote trans-cleavage by Cas13.

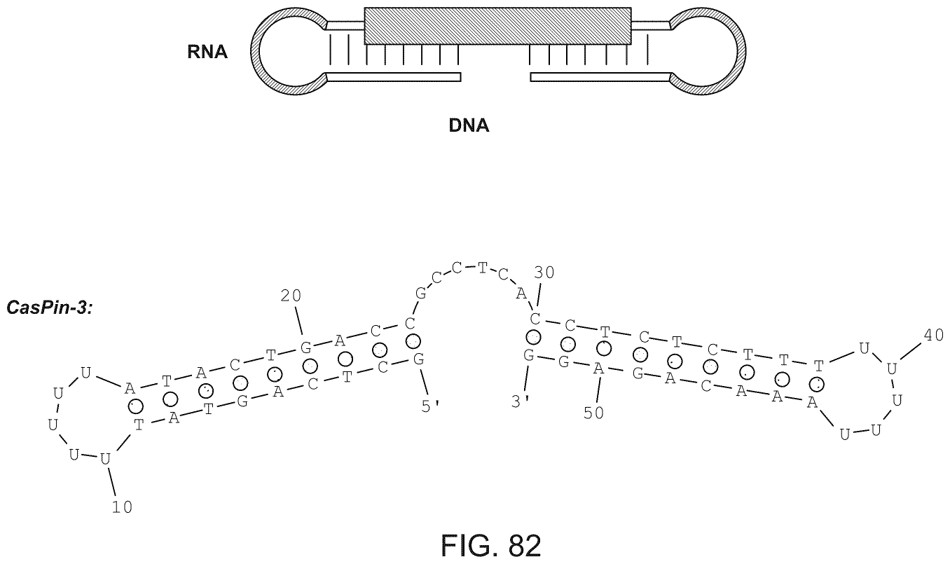

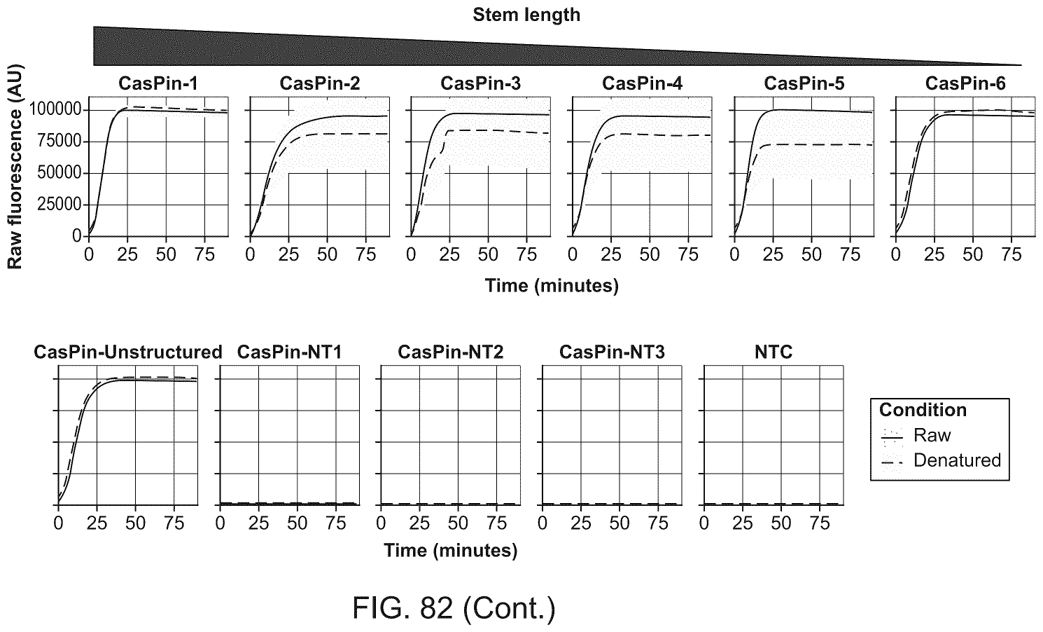

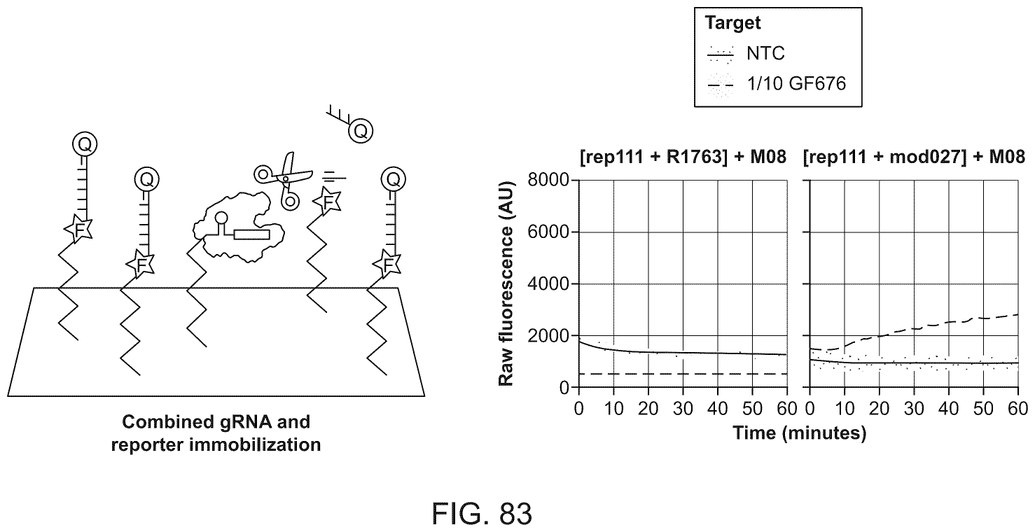

In some embodiments, two hairpins are used on either end of the target site. FIG. 82 presents results for such an embodiment and indicates capability for blocking Cas13 from recognizing the ssDNA target site. In some embodiments, CasPin oligos have varying lengths of hairpin stems. In some embodiments, CasPin oligo do not have stem structures. In some embodiments, CasPin oligos contain another DNA sequence. Such embodiments were evaluated and found to not be recognized by the crRNA. Both raw oligos from the manufacture and those that had been denatured and refolded at 25 C in a Cas13 DETECTR reaction were tested. The results of this experiment, shown in FIG. 84, demonstrate that Cas13 was able to recognize the target site regardless of the stem length. In some embodiments, longer stem length oligos block Cas13 recognition without RNA cleavage to release the structure. One-pot DETECTR on handheld microfluidic device

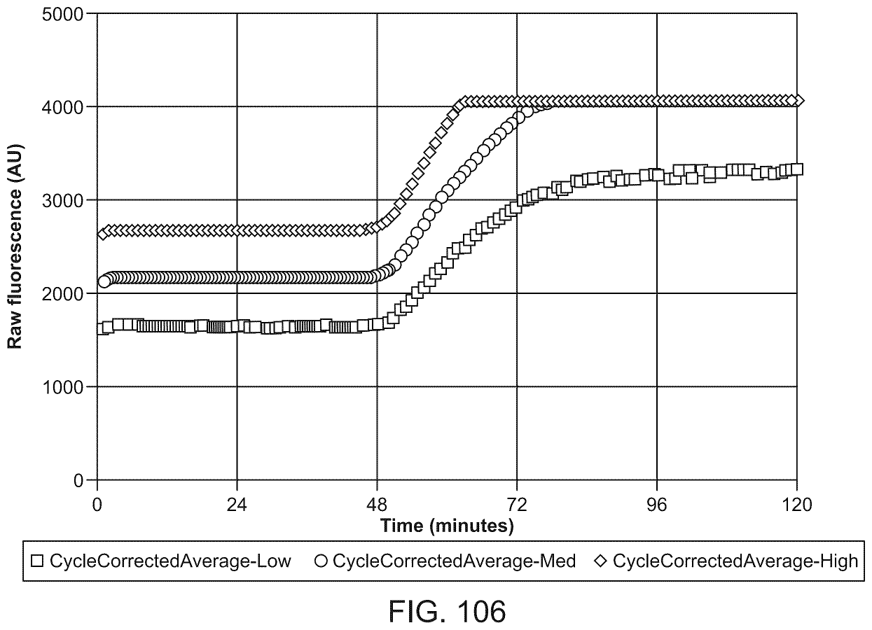

Described herein are various devices and methods for running one-pot DETECTR assays on a handheld device. Any of the devices described herein may be configured to perform a one-pot DETECTR assay. For example, the device shown in FIGS. 10A-12 may be configured to run a one-pot DETECTR assay. Results for such an embodiment are shown in FIG. 106. The y-axis displays raw fluorescence (AU) and the x-axis shows time in minutes. The three traces show the same data collected at three different acquisition settings. The line trace represented by squares, a low cycle corrected average, shows a setting that did not saturate the detector and thus shows the full dynamic range of the signal throughout the assay.

### Multiplexed DETECTR Assay-Based Lateral Flow Assay

Described herein are various methods of multiplexing detection. Any of the devices described herein may be configured for multiplexing (e.g., detecting multiple target nucleic acids). In certain instances, multiplexed detection may utilize one or more lateral flow assay strips.

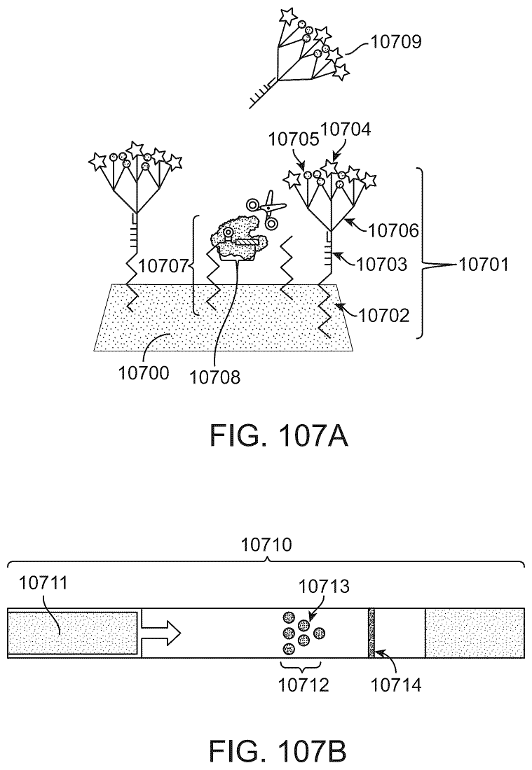

Described here are various devices and methods for a DETECTR™ assay based multiplex lateral flow strip as illustrated in FIG. 107. In some embodiments, reporters (10701) are immobilized to a surface (10700) of a solid support. In some embodiments, programmable nuclease (e.g., Cas-complex) probes (10707) are immobilized to a surface (10700). In some embodiments, programmable nuclease probes (10707) comprise guide nucleic acid such as a single guide RNA (sgRNA) (10708). In some embodiments, a programmable nuclease probe (10707) may comprise a sgRNA (10708) that is designed to be a compliment for a target nucleic acid of a sample. In some embodiments, programmable nuclease probes (10707) and reporters (10701) are both immobilized to a surface (10700). In some embodiments, programmable nuclease probes (10707) and reporters (10701) are both immobilized to a surface (10700) in close enough proximity that the reporter (10701) can be cleaved by the programmable nuclease of the programmable nuclease probe (10707). In some embodiments, programmable nuclease probes (10707) and reporters (10701) are both immobilized to a surface (10700) in close enough proximity that the reporter (10701) can be cleaved (10709) by the programmable nuclease of the programmable nuclease probe (10707) upon binding of a target nucleic acid to an sgRNA (10708) of the programmable nuclease probe (10707) when target nucleic acid and sgRNA (10708) are compliments. In such an embodiment, this indicates the presence of and is a “hit” for the target. In some embodiments, binding of a target nucleic acid that is complimentary to a sgRNA (10708) of the programmable nuclease probe (10707) results in the programmable nuclease of the programmable nuclease probe (10707) initiating cleavage of nucleic acids within a close enough proximity of the programmable nuclease. In some embodiments, the surface (10700) is in the bottom of a well. In some embodiments, a collection of a first programmable nuclease probe (10707) and a first reporter (10701) are immobilized to a surface at one location of the surface (10700).

In some embodiments, as illustrated in FIG. 107A, the reporter (10701) may comprise a surface linker (10702), a nucleic acid (10703), a second linker (10706), a detection moiety (e.g., a label) (10704), and an affinity molecule (e.g., a binding moiety) (10705). In some embodiments, the binding moiety (10705) is biotin. In some embodiments, there is more than one copy of the same reporter (10701) immobilized to the surface.

In some embodiments, lateral flow assay strips (10710) are used to detect cleaved reporters (10709). In some embodiments, cleaved reporters (10709) are contacted to the sample pad (10711) of the lateral flow strip (10710). In some embodiments, the cleaved reporters (10709) bind to conjugate particles present in the sample pad. In some embodiments, the conjugate particles are gold nanoparticles. In some embodiments, the gold nanoparticles are functionalized with anti-biotin. In some embodiments, the anti-biotin functionalized gold nanoparticles bind to the cleaved reporter which contains one or more biotins in the binding moiety (10705).

In some embodiments, the reporter contains a second linker. In some embodiments, the second linker links one or more binding moieties to the nucleic acid. In some embodiments, the second linker links one or more labels to the nucleic acid. In some embodiments, the second linker links both one or more binding moieties and one or more labels to the nucleic acid of the reporter. In some embodiments, the reporter is a dendrimer or trebler molecule.

In some embodiments, the reporter contains a label. In some embodiments, label is FITC, DIG, TAMRA, Cy5, AF594, Cy3, or any appropriate label for a lateral flow assay.

In some embodiments, the reporter may comprise chemical functional group for binding. In some embodiments, the chemical functional group is biotin. In some embodiments, the chemical functional group is complimentary to a capture probe on the flowing capture probe (e.g., conjugate particle or capture molecule). In some embodiments, the flowing capture probe is a gold nanoparticle functionalized with anti-biotin. In some embodiments, the flow capture probe is located in the sample pad. In some embodiments, the flowing capture probe is located in a conjugate pad in contact with the sample pad, wherein both lateral flow assay strip may comprise both the sample pad and conjugate pad, further wherein both the sample pad and the conjugate pad are in fluid communication with the detection region.

In some embodiments, the lateral flow assay strip (10710) contains a detection region (10712). In some embodiments, the detection region (10712) may comprise one or more detection spots. In some embodiments, the detection spots contain a stationary capture probe (e.g., capture molecule). In some embodiments, the stationary capture probe may comprise one or more capture antibodies. In some embodiments, the capture antibodies are anti-FITC, anti-DIG, anti-TAMRA, anti-Cy5, anti-AF594, or any other appropriate capture antibody capable of binding the detection moiety or conjugate.

In some embodiments, the flowing capture probe comprising FITC is captured by a stationary capture probe comprising anti-FITC antibody. In some embodiments, the flowing capture probe comprising TAMRA is captured by a stationary capture probe comprising anti-TAMRA antibody. In some embodiments, the flowing capture probe comprising DIG is captured by the stationary capture probe comprising anti-DIG antibody. In some embodiments, the flowing capture probe comprising Cy5 is captured by the stationary capture probe comprising anti-Cy5 antibody. In some embodiments, the flowing capture probe comprising AF574 is captured by the stationary capture probe comprising anti-AF594 antibody.

In some embodiments, the lateral flow assay strip (10710) may comprise a control line (10714). In some embodiments, the control line (10714) may comprise anti-IgG that is complimentary to all flowing capture probes. In some embodiments, when a flowing capture probe does not bind to a reporter the flowing capture probe will be captured by the anti-IgG on the control line, ensuring the user that the device is working properly even no signal is read from the test line.

In some embodiments, the lateral flow assay strip (10710) may comprise a sample pad. In some embodiments, the flowing capture probe may comprise anti-biotin. In some embodiments, the flowing capture probe may comprise HRP. In some embodiments, the flowing capture probe may comprise HRP-anti-biotin. In some embodiments, the flowing capture probe is HRP-anti-biotin DAB/TMB.

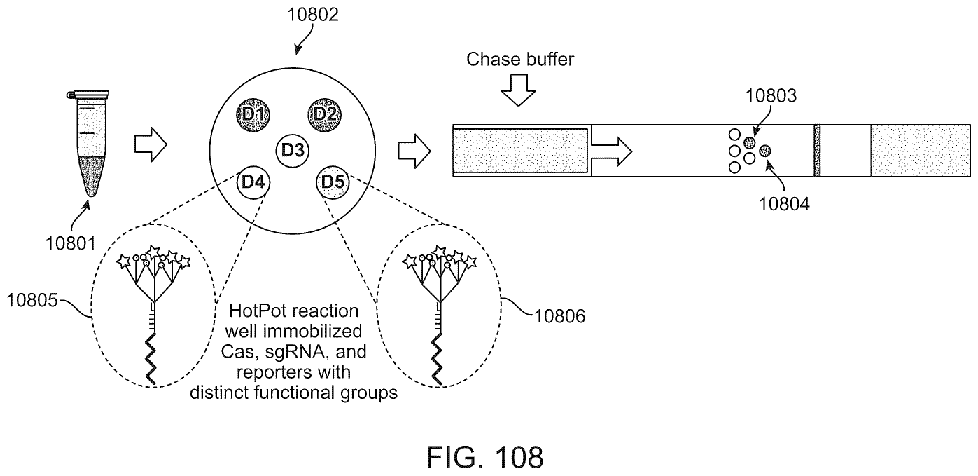

Described here are various devices and methods for a DETECTR™ assay based multiplex lateral flow strip as illustrated in FIG. 108. FIG. 108 depicts a non-limiting exemplary workflow for a DETECTR™ assay read out on a lateral flow assay strip. In some embodiments, a sample (10801) contains one or more target nucleic acid sequences. In some embodiments, a sample (10801) (e.g., a sample solution) contains at least first and second target nucleic acid sequences. In some embodiments, the sample (10801) is introduced into a well (10802) (e.g., D1-D5) where at one or more locations there are different guide nucleic acids such as sgRNAs immobilized to the surface of the well. In some embodiments, the sgRNAs are part of a programmable nuclease probe immobilized to a surface. In some embodiments, a sgRNA is designed to specifically bind to a target nucleic acid in the sample. In some embodiments, there are different sgRNAs corresponding to different locations (e.g., locations D1-D5) on the surface of the well, where each different sgRNA is complimentary for a different target nucleic acid sequence that may or may not be present in the sample. In some embodiments, in addition to the programmable nuclease probes containing sgRNAs, each location is functionalized with one or more reporter probes having distinct functional groups. In some embodiments, the reporter probes are in close enough proximity to be cleaved by the programmable nuclease probes. In some embodiments, as described in example 13, binding between a particular sgRNA and the target nucleic acid to which the sgRNA is designed to specifically bind allows for a section of one or more reporters are cleaved from a corresponding nucleic acid and released into the sample solution. In some embodiments, the reporter is functionalized with a label. In some embodiments, the lateral flow assay strip contains a detection region comprising detection spots (e.g., 10803, 10804), where each detection spot contains a different type of capture antibody. In some embodiments, each capture antibody type specifically binds to a particular label type of a reporter. In some embodiments, a first detection spot (10803) contains the capture antibody anti-FITC. In some embodiments, location D5 on the surface of the well (10802) contains a first immobilized programmable nuclease probe including the sgRNA specific to the first target nucleic acid sequence. In some embodiments, D5 additionally contains the immobilized first reporter (10806), which is labeled with FITC. In some embodiments, upon binding of the first target nucleic acid sequence to the programmable nuclease probe causes the cleavable nucleic acid of the first reporter (10806) to be cleaved and released into solution. Alternatively, or in combination, in some embodiments, a second detection spot (10804) contains the capture antibody anti-DIG. In some embodiments, a second location D4 contains the immobilized programmable nuclease probe including the sgRNA specific to the second target nucleic acid sequence. In some embodiments, D4 additionally contains the immobilized second reporter (10805), which is labeled with DIG. Therefore, in some embodiments, upon binding of the second target nucleic acid sequence, the immobilized second reporter (10805) is cleaved and released into solution. In some embodiments, the solution containing cleaved first and second reporters (10805) and (10806) is contacted to the sample pad of the lateral flow assay strip along with chase buffer. In some embodiments, the sample pad has one or more flowing capture probes (e.g., anti-biotin-AuNP) disposed thereon. In some embodiments, the sample solution containing the cleaved first and second reporters, along with the chase buffer, flow across the sample pad, where the reporters are bound to conjugates (e.g., anti-biotin-gold nanoparticles). In some embodiments, the solution containing the cleaved reporters is contacted to the sample pad by manually pipetting. In some embodiments, the solution containing the cleaved reporters is contacted to the sample pad being drawn from a chamber in fluid connection with the sample pad. In some embodiments, the solution containing the cleaved reporters is contacted to the sample pad by being drawn from a chamber in which the assay resulting in the cleaved reporter solution occurs. In some embodiments, the reporters are cleaved in the sample pad. In some embodiments, the reporters are cleaved in the sample pad by a DETECTR™ assay. In some embodiments, the solution is drawn into and out of the sample pad by capillary action, or wicking. In some embodiments, the capillary action or wicking is caused by the liquid being drawn into an absorption pad. In some embodiments, the capillary action or wicking is caused by the liquid being drawn into an absorption pad, not requiring electrical power. In some cases, the solution is drawn into or out of the sample pad by a pressure gradient. In some embodiments, the gold nanoparticle-reporter conjugates having reporter (10806) labeled with FITC will selectively bind to the first detection spot (10803) containing the capture antibody anti-FITC, thus indicating the presence of the first target nucleic acid sequence in the sample. In some embodiments, the AuNP-reporter conjugates having mostly reporter (10805) labeled with DIG will selectively bind to the second detection spot (10804) containing the capture antibody anti-DIG, thus indicating the presence of the second target nucleic acid sequence in the sample. In this manner, for some embodiments, parallel detection of two or more target nucleic acid sequences present in a multiplexed sample is enabled.

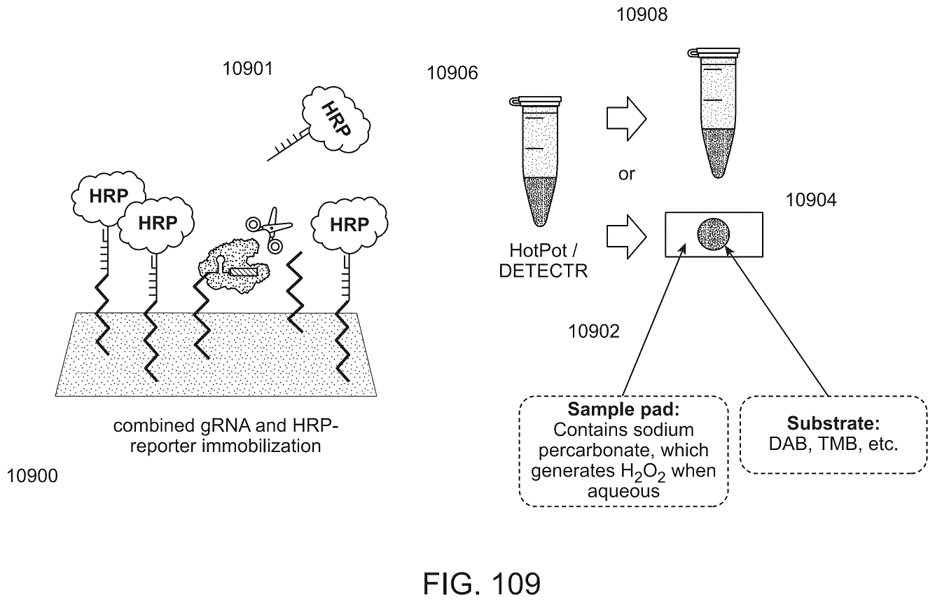

Described herein are various embodiments of lateral flow-based detection as illustrated in FIG. 109. In some embodiments, horse radish peroxidase (HRP) (10901) is used to enhance detection in lateral flow based DETECTR™ assays. In some embodiments, a sample containing a target(s) nucleic acid sequence is exposed to a surface (10900) upon which programmable nuclease probes and reporter probes are immobilized on the surface. In some embodiments, the reporter probes contain HRP molecules. In some embodiments, upon cleavage of the reporter by the programmable nuclease following a specific binding event between the target and the guide RNA, the cleaved portion of the reporter is released into the sample solution (10906). In some embodiments, the sample solution is then exposed to a lateral flow assay strip (10902) comprising or adjacent to a sample pad containing sodium percarbonate (10904), which generates H₂O₂ when exposed to an aqueous solution. In some embodiments, the rehydration of the sodium percarbonate to form H₂O₂ occurs when the sample is wicked through the region. In some embodiments, the substrate contains DAB, TMB, or any other sufficient substrate. In some embodiments, the “spot” changes from blue to red, indicating the presence of HRP, and in turn a “hit” for the target nucleic acid sequence. In some embodiments, the readout is accomplished in solution, upon a color change of the sample solution (10908).

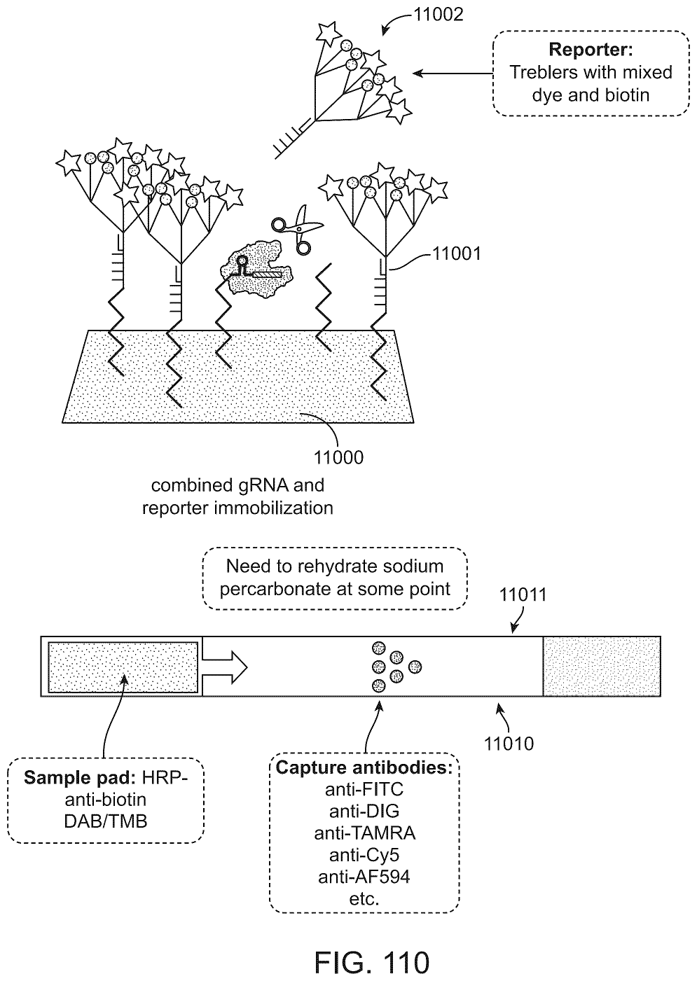

Described here are various methods and devices utilizing HRP-enhanced multiplexed DETECTR™ assays utilizing lateral flow assay strips for readout. In some embodiments, an HRP-signal enhanced multiplexed lateral flow assay as illustrated in FIG. 110. In some embodiments, the immobilized surface (11000) of a support medium and detection on the lateral flow assay strip (11010) are carried out as described in FIGS. 107A-110 with the exception that signal enhancement is not carried out by gold nanoparticles scattering light. Instead, in some embodiments, the anti-biotin labeled AuNP are supplanted by HRP-anti-biotin DAB/TMB. In some embodiments, the HRP is activated by sodium percarbonate present in the lateral flow assay strip which is rehydrated by the reaction and or chase buffer. In some embodiments, HRP allows for strong enough signal so as not to require sample amplification such as PCR.

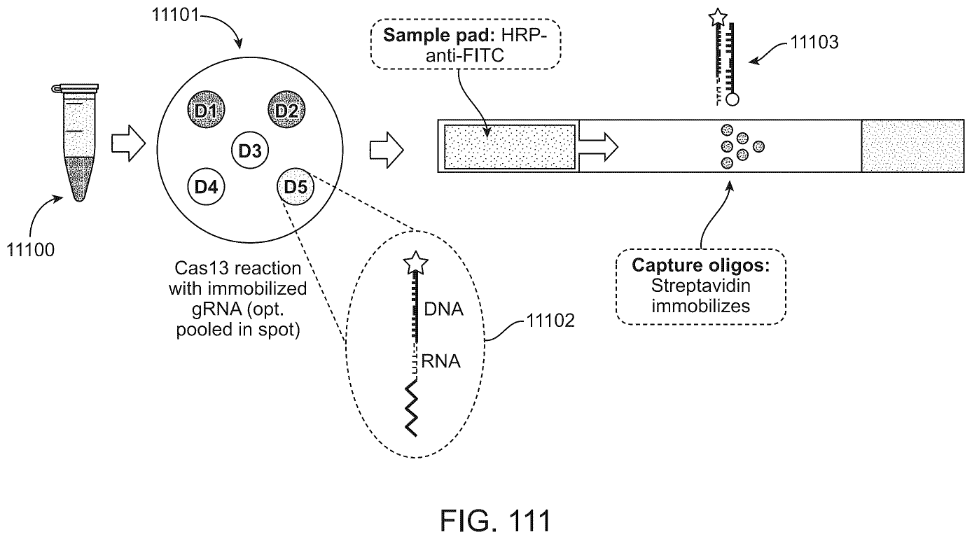

Described herein are various embodiments for multiplexed target nucleic acid detection utilizing Cas13 RNA cleaving specificity over DNA, HRP-signal enhancement, and capture oligo probe specificity. In some embodiments, as shown in FIG. 111, the sample (11100) contains different target nucleic acids. In some embodiments, the sample (11100) is then contacted to the surface of the well (11101) that is functionalized at one or more locations (e.g., five locations, D1-D5). In some embodiments, there are one or more locations. In some embodiments, Cas13 enzyme is present in the programmable nuclease probe. In some embodiments, Cas13 cleaves RNA but not DNA, enabling the use of a reporter (11102) that contains nucleic acid sequences with both DNA and RNA strands. In some embodiments, upon binding of the target nucleic acid to the sgRNA, the RNA of the reporter is cleaved by the Cas13 enzyme and a fragment containing a portion of the RNA, the complete DNA sequence, and a (FITC label is released into solution. In some embodiments, this action is repeated in parallel at each location, or spot with different reporters. In some embodiments, this action is repeated in parallel at locations D1 through D5 for five different target nucleic acids, producing five distinct reporter fragments. In some embodiments, the solution is then contacted to the sample pad of the lateral flow assay strip, where the sample pad contains HRP-anti-FITC. In some embodiments, the FITC-labeled reporter fragment then binds to the HRP-anti-FITC, forming a complex (11103) and is carried downstream across the detection region, binding specifically to the detection spot containing a capture oligo that has been designed to be the compliment for the oligo in the complex (11103).

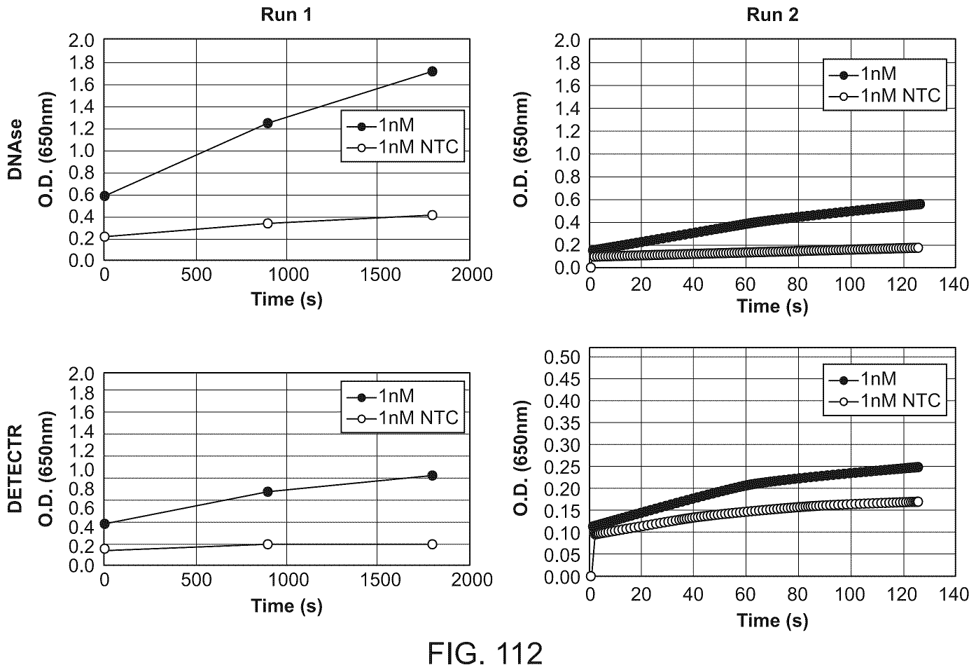

FIG. 112 shows results for both DNAse and DETECTR based assays for two replicate runs performed a week apart.

### Guide RNA Pooling for Signal Enhancement

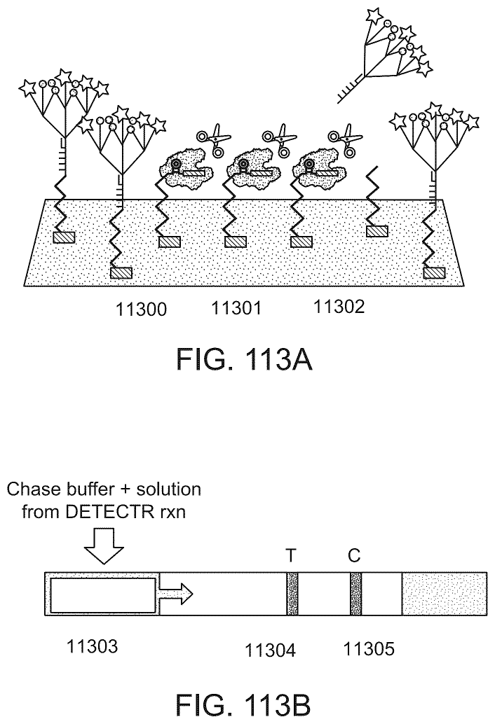

In some embodiments, one or more programmable nuclease probes (11300-11302) are used for guide pooling to achieve enhanced signal detection in lateral flow assays as shown in FIG. 113A. In some embodiments, a first programmable nuclease probe (11300) may comprise a first sgRNA that is complimentary for a first segment of a target nucleic acid. In some embodiments, a second programmable nuclease probe (11301) may comprise a second sgRNA that is complimentary for a second segment of a same target nucleic acid. In some embodiments, a third programmable nuclease probes (11302) may comprise a third sgRNA that is complimentary for a third segment of the same target nucleic acid. In some embodiments, the first programmable nuclease probe, the second programmable nuclease probe, and the third programmable nuclease probe are all located close enough to allow for sufficient cleaving of a reporter that is labeled to indicate the presence of the target nucleic acid. FIG. 113B shows a typical lateral flow assay strip comprising a sample pad (11303), a test line (11304), and a control line (11305).

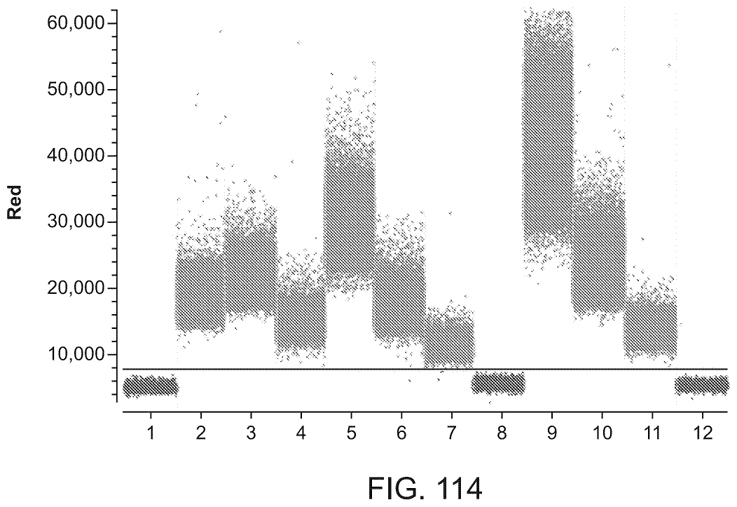

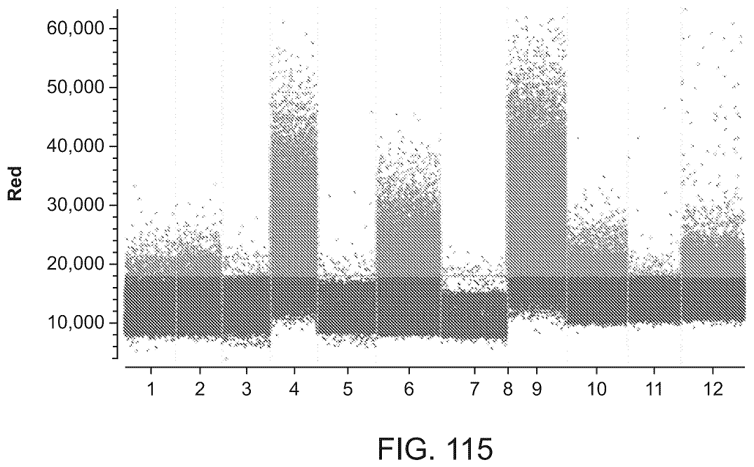

Described herein are various embodiments of guide pooling to achieve enhanced signal detection in lateral flow assays. For some embodiments as described herein, guide pooling shows enhanced Cas12a activity. FIG. 114 (described in Example 20) depicts results of a DETECTR™ assay showing enhanced Cas12a-based detection of the GF184 target using a pooled-guide (pooled-gRNA) format compared to DETECTR™ Cas12a-based assay using an individual gRNA format. In FIG. 114 the y-axis, labeled “Red” displays units of intensity and the x-axis shows the chamber number wherein a different DETECTR™ reaction occurred. FIG. 115 (described in Example 20) depicts results of a DETECTR™ assay showing enhanced sensitivity of the Cas13a-based detection of the SC2 target using a pooled-guide format compared to the Cas13a-based assays using an individual guide format.

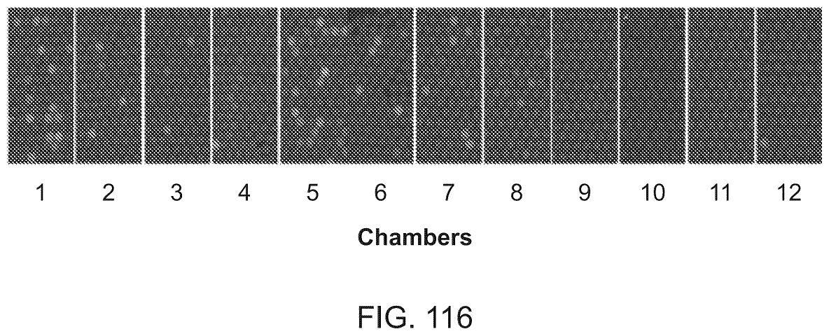

FIG. 116 (described in Example 20) shows images corresponding to each chamber, used to count the number of positive droplets, showing that the Cas13a-DETECTR™ assay samples containing the pooled guide RNAs generated more crystals containing the amplified products per starting copy of the target RNA than the Cas13a-DETECTR™ assay samples containing the guide RNAs in individual format.

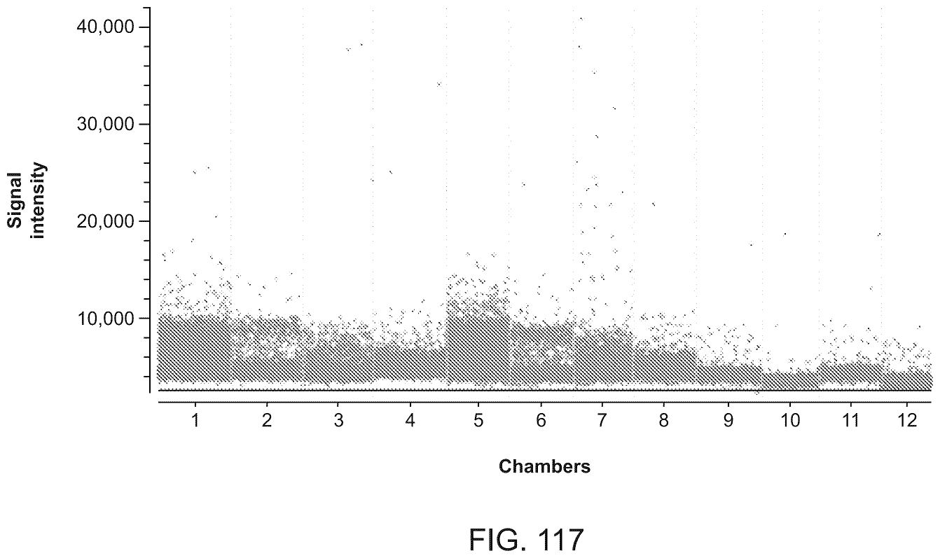

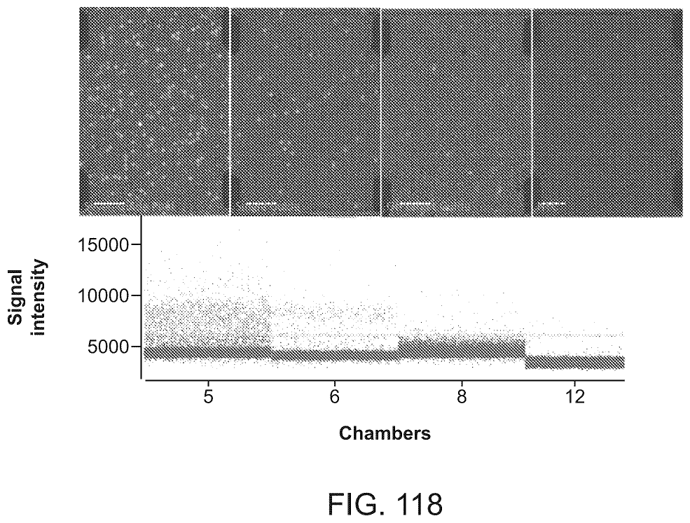

FIG. 117 (described in Example 20) shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 118 (described in Example 20) shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 118 also shows that relative quantification performed by counting the number of positive droplets showed that the Cas13a-DETECTR™ assay samples containing the pooled guide RNAs generated more crystals containing the amplified products per starting copy of the target template RNA than the Cas13a-DETECTR™ assay samples containing the guide RNAs in individual format.

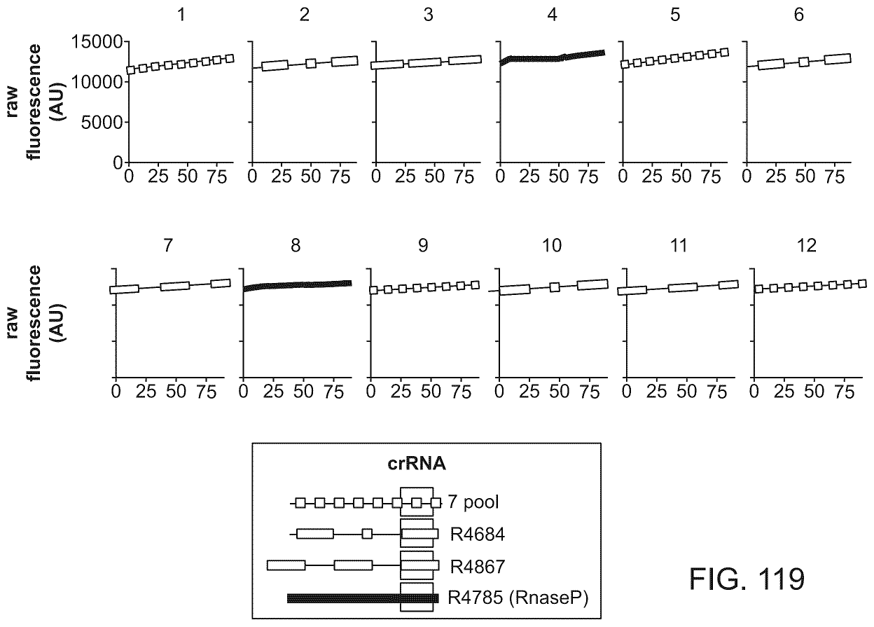

FIG. 119 (described in Example 20) shows that Cas13a DETECTR™ assay samples containing the pooled guides (R4637, R4638, R4667, R4676, R4684, R4689, R4691) did not exhibit higher target detection sensitivity per starting copy of the target than the Cas13a DETECTR™ samples containing the single guides R4684, R4667, or R4785 (RNAseP guide) in individual format.

### DETECTR Based Multiplexed Lateral Flow PON Device

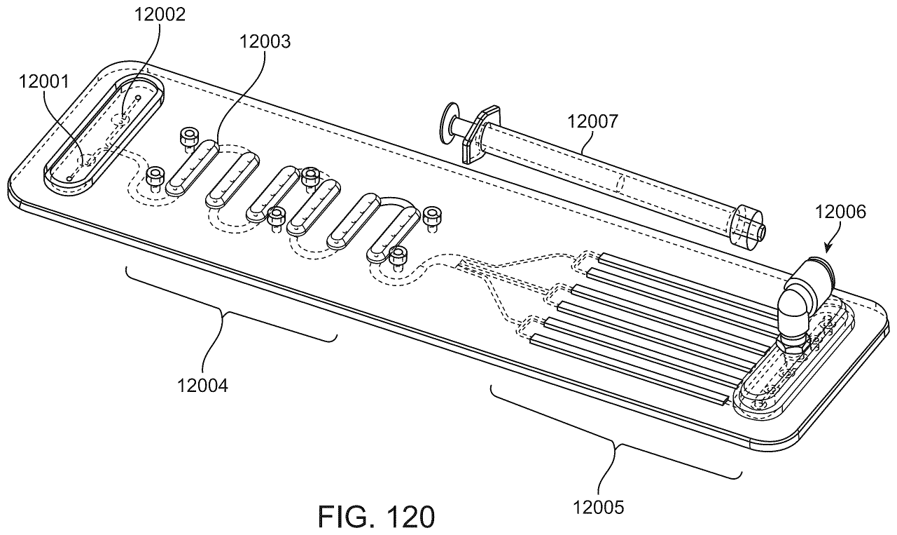

Described herein are various methods and devices for a programmable nuclease (e.g., DETECTR) assay based multiplex lateral flow assay as illustrated in FIG. 120. FIG. 120 depicts a non-limiting exemplary handheld device for performing a multiplexed programmable nuclease (e.g., DETECTR) assay. In some embodiments, a sample contains one or more target nucleic acid sequences. In some embodiments, the sample is introduced into a sample input (12001) (also referred to herein as a sample interface) and loaded into one or more zones (e.g., heating regions or reaction chambers, 12003) comprising one or more dried or lyophilized programmable nuclease reagents (e.g., immobilized to a glass bead). In some embodiments, the sample interface may be configured to concentrate, filter, lyse, or otherwise prepare the sample as described herein. In some embodiments, negative pressure is applied to a negative pressure port (12006) by a negative pressure source (e.g., a syringe, 12007) in order to load the DETECTR zones with the sample. In some embodiments, the sample is a lysis sample. In some embodiments, a plurality of DETECTR zones (12003) are coupled to one another along a serpentine fluid path (12004). In some embodiments, the fluid path may be spiral as described herein. In some embodiments, amplification of one or more target nucleic acids is performed in an amplification region comprising the one or more DETECTR zones (12003). The amplification region (12004) may be heated (e.g., to 55 C for 20 minutes or less) to amplify the one or more target nucleic acids as described herein. In some embodiments, the amplification region (12004) may comprise interspersed polymers. Heating can include liquid-phase chemical heating, solid phase chemical heating, and electric heating (including, but not limited to, resistance/Joule heating, induction heating, Peltier heat pumping, etc.). In some embodiments, the device includes multiple lateral flow strips (12005). In some embodiments, a diluent is introduced into a diluent input (12002). Those skilled in the art will understand that the detection region (e.g., lateral flow assay region (12005)) may include any compatible assay or a lateral flow strip as described herein.

In some embodiments, the amplification region (12004) may be coated. In some embodiments, a coating may be a hydrophilic coating, a hydrophobic coating, an inorganic coating, or an organic coating. In some embodiments, a coating may comprise a polymer coating. In some embodiments, a coating may comprise a polyethylene glycol coating. In some embodiments, a coating may comprise a streptavidin coating. In some embodiments, the interspersed polymers may be a crowding agent. In some embodiments, the crowding agent may comprise polyethylene glycol.

In some embodiments, the one or more reaction or heating zones may comprise guide nucleic acids (e.g, sgRNAs) immobilized to a surface (e.g., a glass bead disposed within a DETECTR zone). In some embodiments, the guide nucleic acids are part of a programmable nuclease (e.g., Cas-complex) probe immobilized to a surface. In some embodiments, a guide nucleic acid is designed to specifically bind to a target nucleic acid in the sample. In some embodiments, there are different guide nucleic acids corresponding to different locations on the surface and/or different surfaces in the one or more zones, where each different guide nucleic acid is complimentary for a different target nucleic acid sequence that may or may not be present in the sample. In some embodiments, in addition to the programmable nuclease probes containing guide nucleic acids, each surface location is functionalized with one or more reporters having distinct functional groups. In some embodiments, the reporters may be in close enough proximity to be cleaved by the programmable nuclease probes. In some embodiments, as described in example 13, reporters are cleaved and released into the solution upon binding between a particular guide nucleic acid and the target nucleic acid to which the guide nucleic acid is designed to specifically bind. In some embodiments, reporters are functionalized with a detection moiety (e.g., a label).

In some embodiments, chemical heating may be used. In some embodiments, chemical heating may be used to supply energy to initiate and run reactions. In some embodiments, chemical heating may be used to supply energy to initiate and run programmable nuclease assay reactions. In some embodiments, chemical heating may be used to heat reaction or heating zones. In some embodiments, chemical heating may be used to heat regions, chambers, volumes, zones, surfaces, or areas of a device.

In some embodiments, the device may comprise one or more lateral flow assay strips in a detection region disposed downstream of the amplification region. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, a first lateral flow assay strip contains the capture antibody anti-FITC. In some embodiments, a first DETECTR region or surface location (e.g., within a reaction chamber or heating region) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, upon binding of the first target nucleic acid sequence, the first immobilized reporter is cleaved and released into solution. In some embodiments, the first detection moiety is released into solution and the remainder of the first reporter remains immobilized on the surface. Alternatively, or in combination, in some embodiments, a second lateral flow assay strip contains the capture antibody anti-DIG. In some embodiments, a second DETECTR region or surface location (e.g., within a reaction chamber or heating region) contains the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety (e.g., DIG). Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solution containing the first and second cleaved reporters is transferred from the amplification region to the lateral flow region comprising the first lateral flow assay strip and the second lateral flow assay strip. In some embodiments, a chase buffer or diluent is introduced into a diluent input and negative pressure is applied to the negative pressure port to contact the solution containing the first and second cleaved reporters to the lateral flow assay strips of the lateral flow region, where the reporters are bound to conjugate molecules e.g., anti-biotin-AuNPs. In some embodiments, the AuNP-reporter conjugates having the first reporter labeled with the first detection moiety (e.g., FITC) will selectively bind to a first detection region or spot containing the first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing the second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the amplification region is configured to hold about 200 μL of liquid (e.g., sample solution and reagent(s)). In some embodiments, each lateral flow assay strip is configured to hold about 80 μL of liquid (e.g., sample solution and/or chase buffer). In some embodiments, the device may comprise more than one lateral flow assay strip. For example, the device may comprise two, three, four, five, six, seven, eight, nine, ten, or more lateral flow assay strips. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

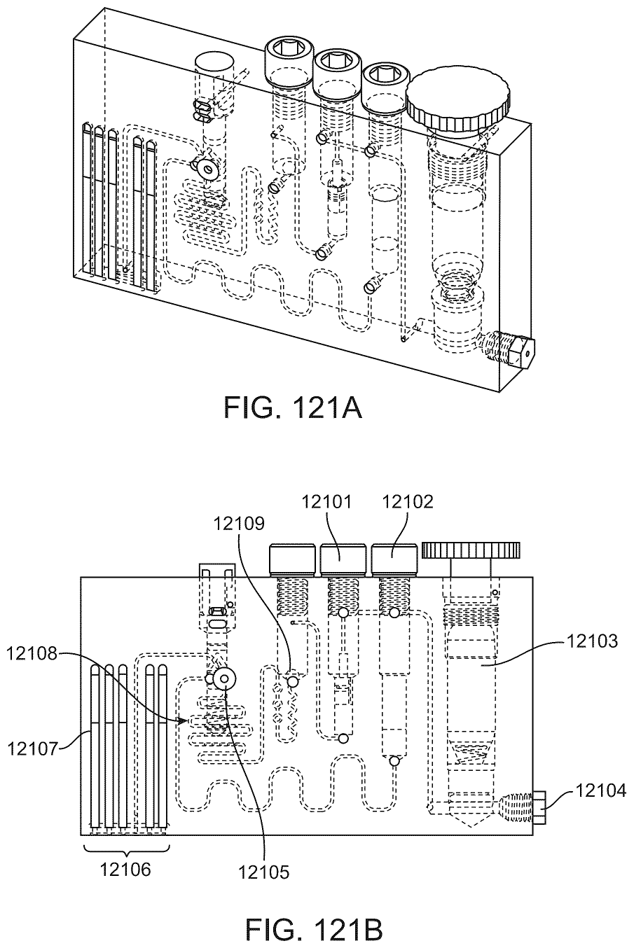

Described herein are various methods and devices for a programmable nuclease assay based multiplex lateral flow assay as illustrated in FIGS. 121A-121B. FIGS. 121A-121B illustrate a non-limiting example of a point of care device for parallel detection of two or more target nucleic acid sequences. In some embodiments, a sample contains one or more target nucleic acid sequences. In some embodiments, the sample is introduced into a sample input chamber (12101) (also referred to herein as a sample interface). The sample input chamber (12101) may be configured to receive a swab comprising the sample. In some embodiments, the sample input chamber (12101) may comprise a solution/buffer (e.g., lysis buffer) to retrieve the sample from the swab. In some embodiments, the sample interface may be configured to concentrate, filter, lyse, or otherwise prepare the sample as described herein. In some embodiments, positive pressure is applied to the sample input chamber (12101) to move the sample solution from the sample input chamber (12101) to a reaction chamber (12108) (e.g., within a heating region) containing one or more dried or lyophilized programmable nuclease (e.g., DETECTR) reagents (12109) (e.g., immobilized to a glass bead) as described herein. In some embodiments, positive pressure may be applied by a positive pressure source (e.g., a compressed gas tank, 12103) in order to load the reaction chamber with the sample solution. In some embodiments, pressure is regulated by a pressure valve (12104) coupled to the positive pressure source (12103). In some embodiments, application of positive pressure (e.g., by operating an actuator to pierce a positive pressure source 12103 comprising a compressed gas tank) moves the sample solution and dried or lyophilized reagent(s) (12109) into a serpentine amplification region of the reaction chamber (12108). In some embodiments, the amplification region may be spiral as described herein. In some embodiments, amplification of one or more target nucleic acids is performed in the amplification region (12108). The amplification region (12108) may be heated (e.g., to 55 C for 20 minutes or less) to amplify the one or more target nucleic acids as described herein. In some embodiments, application of sufficient positive pressure opens the pressure valve (12105) between the amplification region and the detection region, e.g., a lateral flow assay region (12106). In some embodiments, the valve (12105) between the amplification region (12108) and the lateral flow assay region (12106) comprising lateral flow strips (12107) may be opened by pressing a button connected to the valve (12105). Heating can include liquid-phase chemical heating, solid phase chemical heating, and electric heating (including, but not limited to, resistance/Joule heating, induction heating, Peltier heat pumping, etc.). In some embodiments, a diluent or chase buffer may be introduced to the system via the diluent input (12102). In some embodiments, opening the valve (12105) between the amplification region (12108) and the lateral flow assay region (12106) transfers the sample from the amplification region (12108) to the lateral flow assay region (12106). In some embodiments, the sample is mixed with diluent as it moves from the amplification region (12108) to the lateral flow assay region (12106). In some embodiments, the chase buffer pushes the sample from the amplification region (12108) to the lateral flow assay region (12106) without significantly diluting the sample.

In some embodiments, the one or more programmable nuclease reagent(s) comprise guide nucleic acids immobilized to a surface (e.g., a glass bead). In some embodiments, the guide nucleic acids are part of a programmable nuclease (e.g., Cas-complex) probe immobilized to a surface. In some embodiments, a guide nucleic acid may be designed to specifically bind to a target nucleic acid in the sample. In some embodiments, there are different guide nucleic acids corresponding to different locations on the surface and/or different surfaces in the one or more zones, where each different guide nucleic acid is complimentary for a different target nucleic acid sequence that may or may not be present in the sample. In some embodiments, in addition to the programmable nuclease probes containing guide nucleic acids, each surface location may be functionalized with one or more reporters having distinct functional groups. In some embodiments, the reporters are in close enough proximity to be cleaved by the programmable nuclease probes. In some embodiments, as described in example 13, reporters are cleaved and released into the solution upon binding between a particular sgRNA and the target nucleic acid to which the guide nucleic acid is designed to specifically bind. In some embodiments, reporters are functionalized with a detection moiety (e.g., a label).

In some embodiments, the device may comprise one or more lateral flow assay strips in a detection region disposed downstream of the amplification region. Each lateral flow assay strip contains one or more detection regions or spots, where each detection region or spot contains a different type of capture antibody. In some embodiments, each lateral flow assay strip may contain a different type of capture antibody. In some embodiments, each capture antibody type specifically binds to a particular label type of a reporter. In some embodiments, a first lateral flow assay strip contains a first capture antibody (e.g., anti-FITC). In some embodiments, a first surface (e.g., bead) contains the immobilized programmable nuclease (e.g., Cas-complex) including the guide nucleic acid (e.g., sgRNA) specific to the first target nucleic acid sequence. In some embodiments, the first surface additionally contains a first immobilized reporter which is labeled with a first detection moiety (e.g., FITC). In some embodiments, upon binding of the first target nucleic acid sequence, the first immobilized reporter is cleaved and released into solution as described herein. Alternatively, or in combination, in some embodiments, a second lateral flow assay strip contains a second capture antibody (e.g., anti-DIG). In some embodiments, a second surface (e.g., bead) contains the second immobilized programmable nuclease including the guide nucleic acid specific to the second target nucleic acid sequence. In some embodiments, the second surface additionally contains a second immobilized reporter which is labeled with a second detection moiety (e.g., DIG). Therefore, in some embodiments, upon binding of the second target nucleic acid sequence, the second immobilized reporter is cleaved and released into solution.

In some embodiments, the solution containing the first and second cleaved reporters is transferred from the amplification region to the lateral flow region comprising the first lateral flow assay strip and the second lateral flow assay strip. In some embodiments, a chase buffer or diluent is introduced into a diluent input or reservoir and negative pressure is applied to the negative pressure port to contact lateral flow region. A pressure valve may be disposed between the amplification region and the lateral flow region in order to regulate flow of the sample solution from the amplification region to the lateral flow region before amplification has occurred. Actuation of the pressure valve enables the solution containing the first and second cleaved reporters to contact the lateral flow assay strips of the lateral flow region, where the reporters are bound to conjugate molecules, e.g., anti-biotin-AuNPs. In some embodiments, the AuNP-reporter conjugates having the first reporter labeled with the first detection moiety (e.g., FITC) will selectively bind to a first detection region or spot containing a first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing a second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the amplification region is configured to hold about 200 μL of liquid (e.g., sample solution and reagent(s)). In some embodiments, each lateral flow assay strip is configured to hold about 80 μL of liquid (e.g., sample solution and/or chase buffer). In some embodiments, the device may comprise more than one lateral flow assay strip. For example, the device may comprise two, three, four, five, six, seven, eight, nine, ten, or more lateral flow assay strips. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

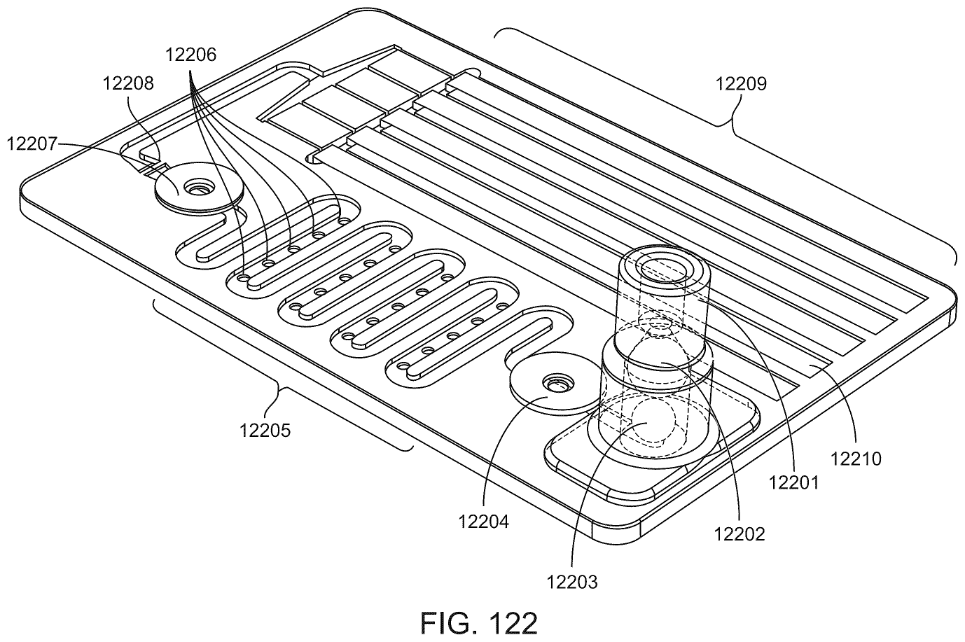

Described herein are various methods and devices for a DETECTR assay based multiplex lateral flow assay as illustrated in FIG. 122. FIG. 122 illustrates an embodiment of a point of care device comprising a lateral flow assay which is substantially similar to the embodiment of FIG. 120 but utilizes positive pressure to drive fluid flow instead of negative pressure. In some embodiments, the device may comprise an input port (12201) that receives a sample. In some embodiments, the input port may be configured to interface with a luer lock syringe containing the sample (e.g., containing a sample solution and/or a swab disposed therein). In some embodiments, the input port (12201) may comprise a ball check valve (12202) configured to seal the input port (12201) until a sample is received. In some embodiments, the device may comprise lyophilized reagents (12203). In some embodiments, the lyophilized reagents (12203) may be stored in the sealed sample interface chamber before the sample is received. In some embodiments, the device may comprise hydrophobic filters that are permeable to gases (12204 and 12207) and configured to permit liquid movement through the device and facilitate release of displaced gases therefrom. In some embodiments, the device may comprise a serpentine channel region (12205). In some embodiments, the serpentine channel region (12205) is heated. In some embodiments, the serpentine channel region (12205) may comprise one or more reaction zones (12206) within a heating region. In some embodiments, the reaction zones (12206) may be configured to perform one or more DETECTR assays. In some embodiments, the reaction zones (12206) comprise amplification enzymes or reagents. In some embodiments, the lyophilized reagents (12203) comprise one or more amplification enzymes or reagents which are reconstituted upon introduction of the sample into the sample interface and which, following reconstitution, may be transferred into the reaction chamber along with the sample. In some embodiments, the sample interface may be configured to concentrate, filter, lyse, or otherwise prepare the sample as described herein. In some embodiments, the reaction zones (12206) comprise visual assays that change color in the presence of a chemical. In some embodiments, the serpentine channel region (12205) is a nucleic acid amplification region. Alternatively, or in combination, the serpentine channel region may be configured to perform one or more programmable nuclease-based detection assays. In some embodiments, a valve may separate the detection region (12209) from the heating region (12205). In some embodiments, the device may comprise a pressure cracking valve (12208) that breaks under sufficient positive pressure and enable fluid movement between the two regions once broken (and, conversely, prevent fluid movement between the regions prior to positive pressure application). In some embodiments, a diluent or chase buffer may be injected into the device via the input port (12201) in order to break the pressure cracking valve (12208) and transfer the sample fluid from the heating/reaction region 12205 to the detection region 12209. In some embodiments, the device may receive positive gas pressure at the input port (12201). In some embodiments, breaking the pressure cracking valve (12208) allows the passage of liquids from the serpentine channel region (12205) to the lateral flow assay region (12209).

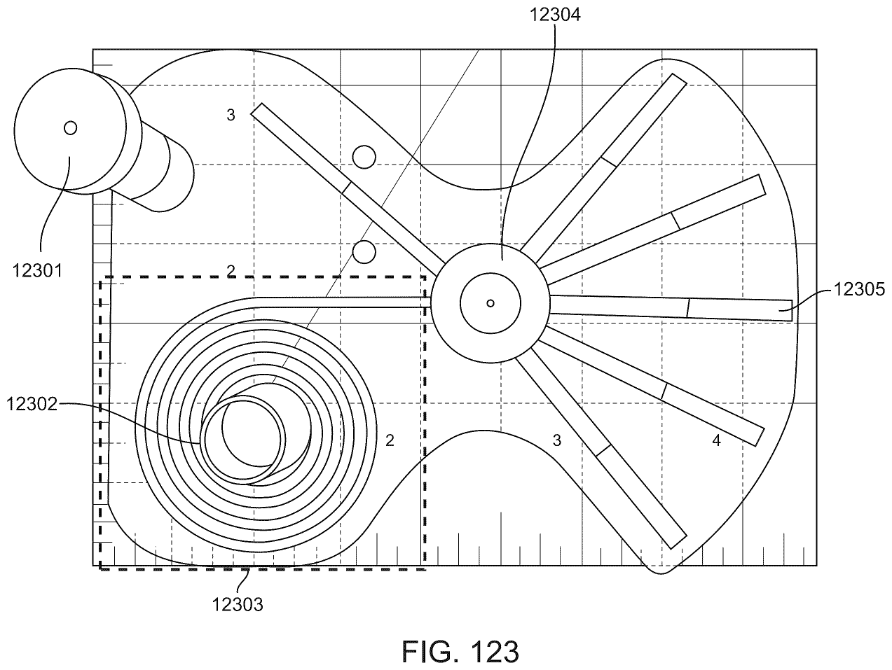

Described herein are various methods and devices for a handheld assay device as illustrated in FIG. 123. FIG. 123 illustrates an embodiment of a handheld assay device comprising an input port (12302), a spiral channel heating or reaction region (12303), a first actuator (12301), a second actuator (12304), and lateral flow assay region (12305). In some embodiments, a nucleic acid sample is delivered at the input port (12302) as described herein, and the sample flows through the spiral channel region (12303). In some embodiments, the sample interface 12302 may be configured to concentrate, filter, or otherwise prepare the sample as described herein prior to transfer into the reaction region 12303. In some embodiments, the first actuator (12301) stores mechanical energy. In some embodiments, the stored mechanical energy is stored as pressurized gas. In some embodiments, the stored mechanical energy is stored in compressed springs. In some embodiments, actuating the first actuator (12301) release the stored mechanical energy. In some embodiments, releasing the stored mechanical energy pushes fluid from the sample interface 12302 through the spiral channel region (12303). In some embodiments, releasing the stored mechanical energy pulls fluid through the spiral channel region (12303). In some embodiments, the spiral channel region may comprise amplification enzymes and reagents. In some embodiments, the sample interface may comprise amplification enzymes and reagents which can be transferred into the spiral channel region with the sample as described herein. In some embodiments, the nucleic acid sample is amplified in the spiral channel region (12303) as described herein. In some embodiments, the spiral channel region is heated. In some embodiments, the second actuator (12304) stores mechanical energy. In some embodiments, the stored mechanical energy is stored as pressured gas. In some embodiments, actuating the second actuator (12304) release the stored mechanical energy. In some embodiments, releasing the stored mechanical energy pushes the sample through the detection (e.g., lateral flow assay) region (12305). In some embodiments, releasing the stored mechanical energy pulls the sample through the lateral flow assay region (12305). The detection region may comprise one or more lateral flow assay strips as described herein.

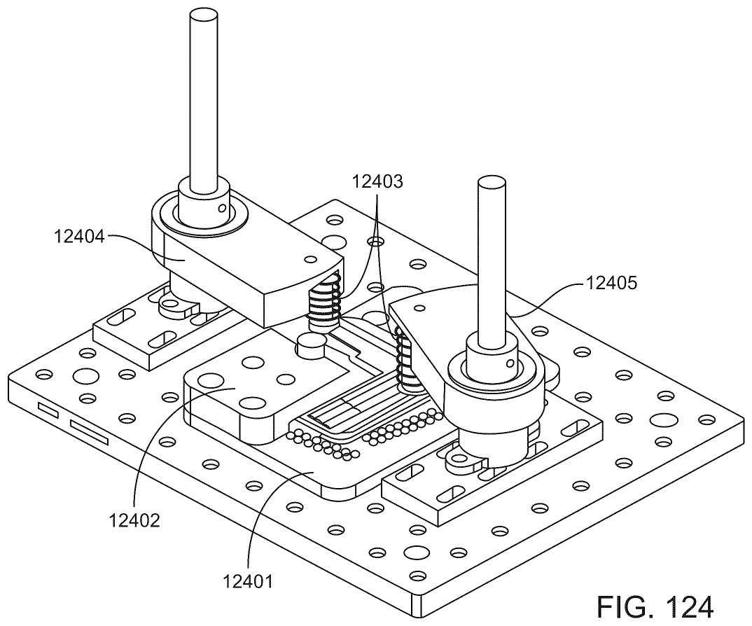

Described herein are various methods and devices for a handheld assay device as illustrated in FIG. 124. FIG. 124 illustrates an embodiment of a handheld assay device breadboard comprising actuator triggers (12404, 12405), actuators (12403), a heating plate (12401), or a platen (12402), which may be configured to be used in conjunction with the fluid flow scheme described in FIG. 123, for example. In some embodiments, the handheld assay device may comprise a single-use, disposable assay component. In some embodiments, the handheld assay device may comprise reusable hardware framework that houses the single-use, disposable assay components (e.g., cartridges). In some embodiments, the single-use, disposable assay components are held by a platen (12402). In some embodiments, the disposable assay components comprise actuators storing mechanical energy as described herein. In some embodiments, the actuators are actuated by actuator triggers (12404, 12405). In some embodiments, the disposable assay components are heated by a heating plate (12401). In some embodiments, the entire device, including triggers, actuators, heating plate, platen, and fluidic components (including lateral flow assay strips, etc.) are coupled together (e.g., within a housing) in a single disposable unit.

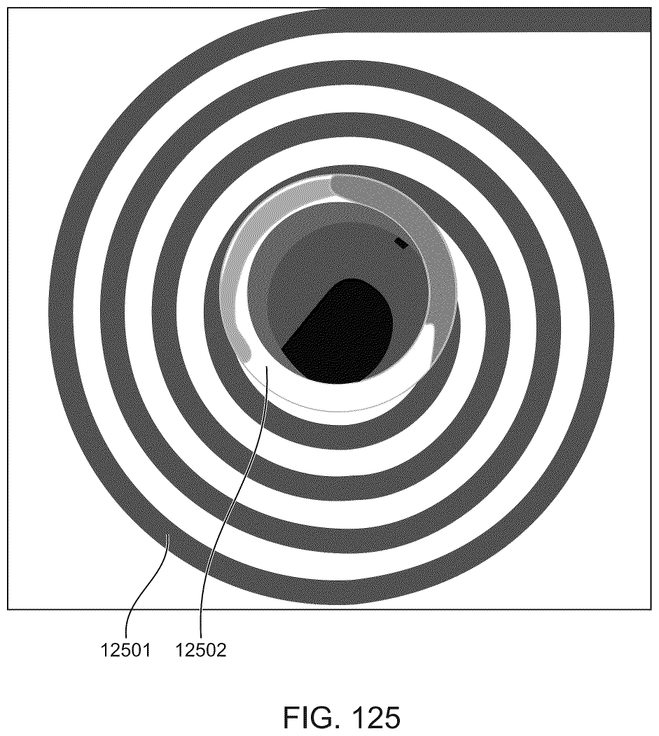

FIG. 125 illustrates an embodiment of a fluid channel (12501) of a handheld assay device. Any of the devices described herein may comprise a spiral fluid channel as shown (e.g., the heating region or reaction region may comprise a spiral fluid channel). In some embodiments, the fluid channel (12501) is substantially spiral in shape. In some embodiments, the fluid channel (12501) is coupled to a port (12502). In some embodiments, the port (12502) is a input sample receiver. In some embodiments, the port (12502) is an input pressure receiver. In some embodiments, the port (12502) is an input diluent receiver. In some embodiments, the fluid channel (12501) may comprise enzymes or reagents. In some embodiments, the fluid channel (12501) is an amplification region. In some embodiments, the fluid channel (12501) is heated. In some embodiments, the fluid channel (12501) may comprise one or more DETECTR assay reagents as described herein.

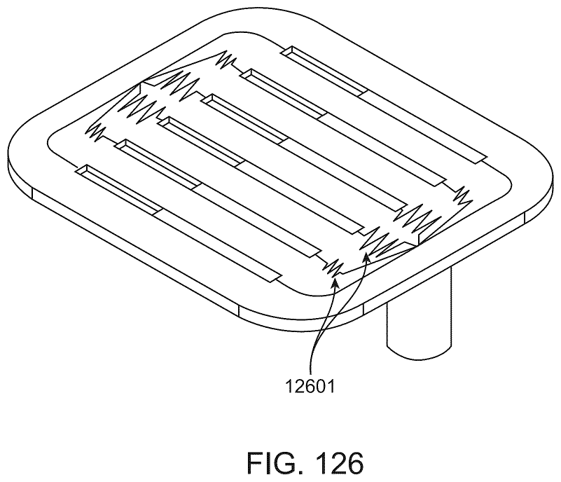

FIG. 126 illustrates an embodiment of a lateral flow assay region of a handheld assay device which may be implemented in any of the devices described herein. In some embodiments, the path length leading to each lateral flow strip is substantially equal in fluid path length (12601) in order to ensure substantially equal fluidic resistances between channels for substantially equal transfer of fluids from the reaction region to the lateral flow assay strips of the lateral flow assay detection region. The fluid path length is defined as the length of the path that is taken by a fluid that moves from one end of a channel to the other end of the channel.

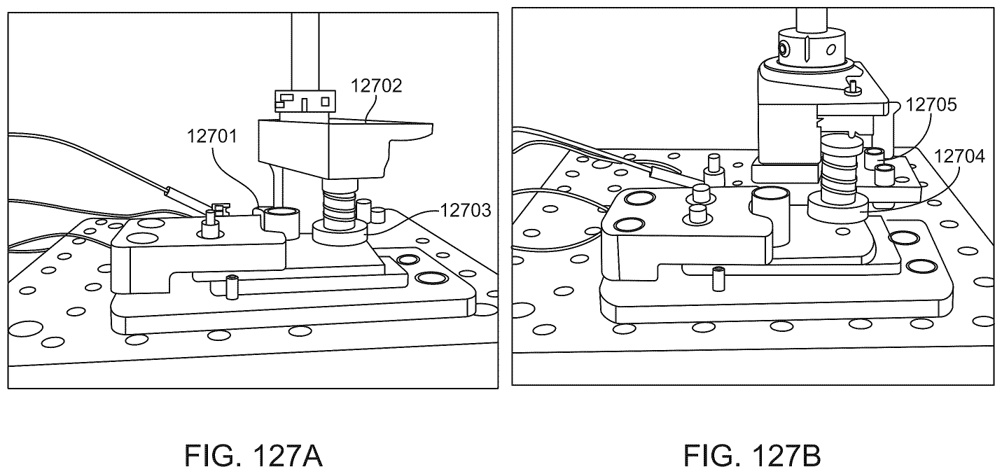

FIGS. 127A-127B show the breadboard device of FIG. 124 in action with the fluidic device of FIG. 123. In FIG. 127A, the spring-based actuator 12703 is loaded and the cartridge is ready for the addition of the sample into the sample interface. In FIG. 127B, the spring-based actuator is partially released and the sample has been pulled from the sample interface into the spiral reaction region. The spring is configured to stay in this position during the amplification and/or programmable nuclease-based detection reaction.

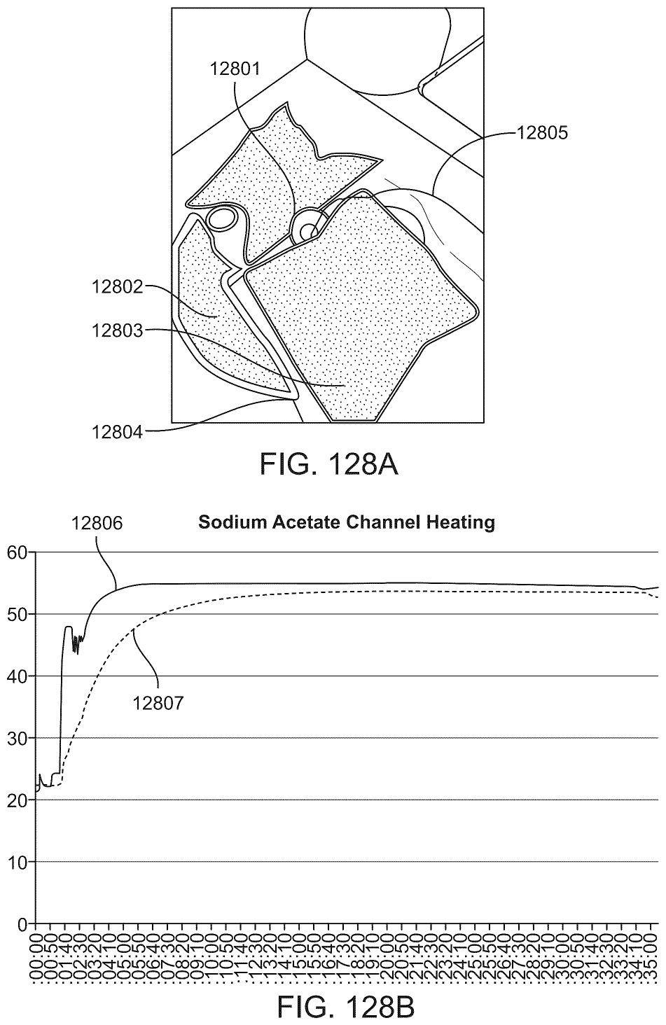

FIG. 128A depicts a chemical heating pouch disposed on the device of FIG. 123 for proof of concept testing. In some embodiments, the chemical heating pouch contains a solution comprising sodium acetate and a metal button, wherein pressing the metal button starts a crystallization reaction that is exothermic. The sample chamber (12801) of the disposable was exposed to allow for sample addition. The sample chamber disposable was nested between heated pouches (12802 and 12803). A temperature probe (12804) nested between the two pouches was used to measure the external temperature. A second temperature probe (12805) was used to measure fluid temperature inside the sample chamber. FIG. 128B depicts the temperature as a function of time measured at the chemical heating pouch (12806) and measured at the second temperature probe (12807). The temperature of the pouch was a couple of degrees hotter than the fluid temperature. In some embodiments, the temperature of the fluid approximately maintained a temperature of 53° C. for about 35 minutes. In some embodiments, the temperature of the fluid approximately maintained a temperature of 55° C. for about 30 minutes.

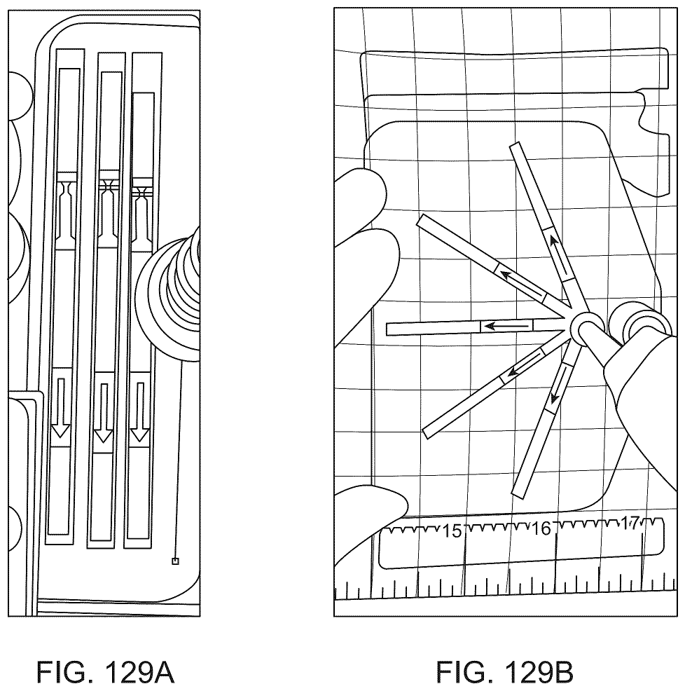

FIG. 129A-129B illustrate additional exemplary embodiments of a lateral flow assay region. In some embodiments, the sample fluid from the heated chamber may be transferred to the lateral flow assay region through aspiration, e.g., created by a syringe vacuum. In some embodiments, the lateral flow assay strips may be arranged in serial geometries as shown in FIG. 129A. In some embodiments, the lateral flow assay strips may be arranged in radial geometries as shown in FIG. 129B. In some embodiments, lateral flow assay strips may be arranged in parallel geometries as shown in FIG. 126. In some embodiments, the lateral flow strips may be used to capture programmable nuclease-mediated reporter cleavage as described herein.

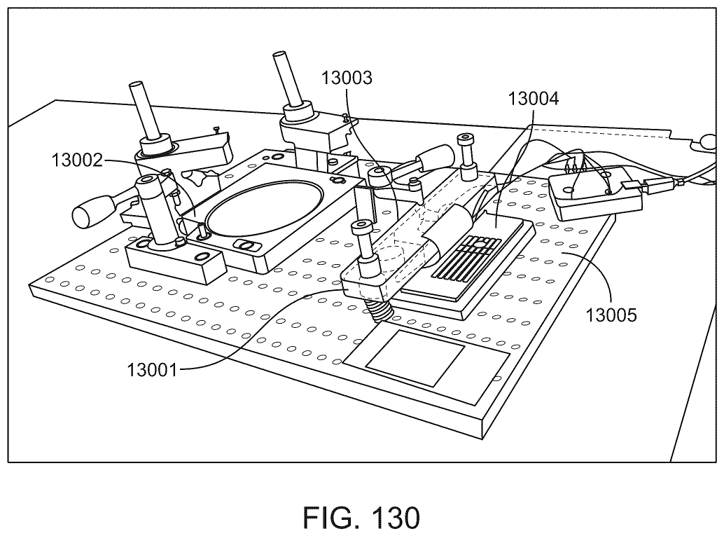

FIG. 130 depicts an embodiment of a breadboard device for testing DETECTR reactions. The breadboard holds 3D printed disposable chips with reaction chamber and lateral flow strips. This embodiment is compatible for testing DETECTR reactions with chemical and electrical heating. In some embodiments, the device may comprise a fluidic drive (13001). In some embodiments, the device may comprise a chemical heating element (13002). In some embodiments, the chemical heating element (13002) may comprise a pouch containing sodium acetate solution and a metal disk, wherein pressing the metal disk triggers the crystallization in the sodium acetate solution. In some embodiments, the temperature of the heating chamber is brought to at least about 50 to at least about 60° C. In some embodiments, the device may comprise a polyamide resistive heater. In some embodiments, the temperature of the heating chamber is brought to at least about 54 to at least about 56° C. In some embodiments, the device may comprise clamps (13003). In some embodiments, the device may comprise lateral flow strips (13004). In some embodiments, the device may comprise electric heating elements (13005). In some embodiments, the device may comprise a polyamide resistive heater.

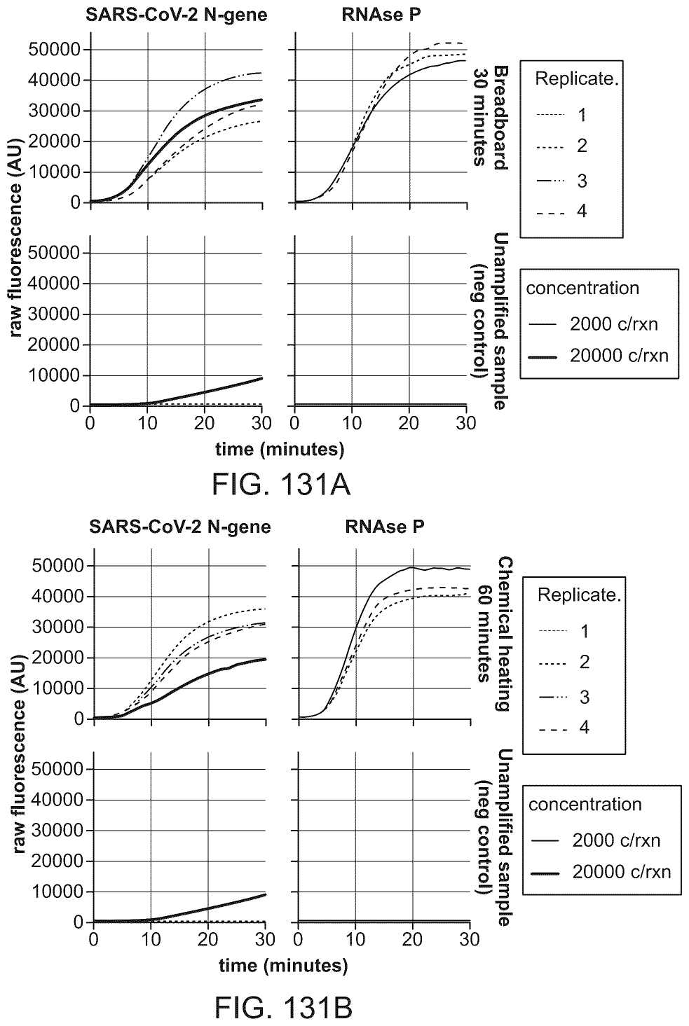

Described herein are various methods and devices for a handheld assay device as illustrated in FIGS. 131A-131B. In FIG. 131A, DETECTR analysis confirmed successful amplification of a duplex assay performed on an embodiment of a handheld device using electric heating. A 2-plex RT-LAMP assay was assembled using 2000 copies of HeLa RNA and 20,000 copies of a synthetic SARS-CoV-2 RNA. The experiment was performed with an assay temperature of 62° C. for 30 minutes. The amplification products were recovered from the chip and a DETECTR assay was performed using a Tecan plate reader to collect the results. A portion of the unamplified mastermix plus sample was stored on ice and used as the negative control for the DETECTR reaction. The resulting DETECTR curves confirm robust amplification had occurred on a spiral breadboard device using electric heating. In FIG. 131B, DETECTR analysis confirmed successful amplification of a duplex assay on an embodiment of a handheld device using chemical heating. A 2-plex RT-LAMP assay was assembled using 2000 copies of HeLa RNA and 20,000 copies of a synthetic SARS-CoV-2 RNA. The experiment was performed with an assay temperature of 55° C. for 60 minutes. The amplification products were recovered from the chip and a DETECTR™ assay was performed using a Tecan plate reader to collect the results. A portion of the unamplified mastermix plus sample was stored on ice and used as the negative control for the DETECTR reaction. The resulting DETECTR curves confirmed robust amplification had occurred on a spiral breadboard device using chemical heating.

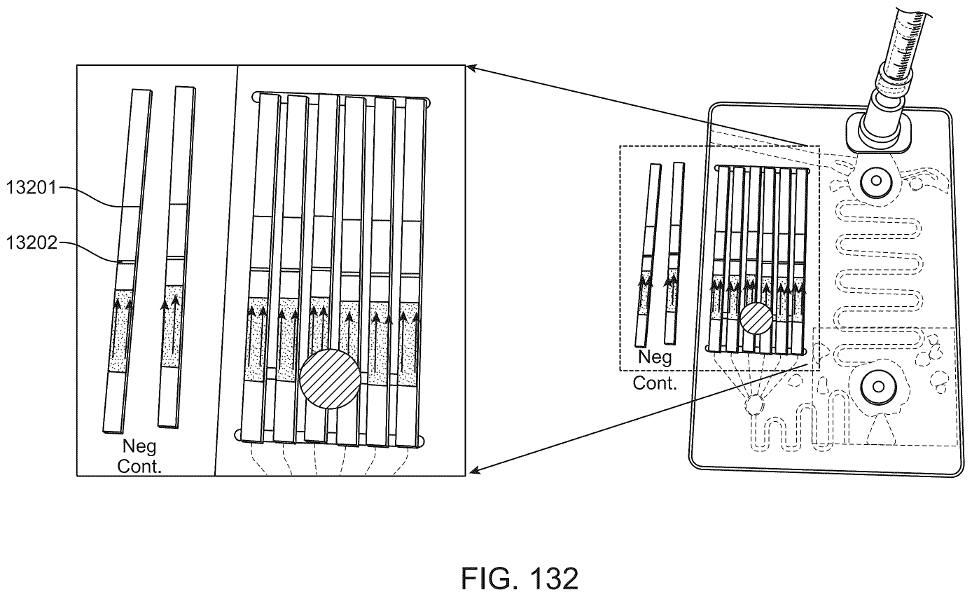

FIG. 132 depicts a DETECTR reaction with lateral flow readout performed on the breadboard device of FIG. 130 with a disposable chip similar to the device shown in FIG. 122. The disposable chip was loaded with 200 μL of DETECTR mastermix containing RT-LAMP amplified SARS-CoV-2 N-gene. The disposable chip was incubated on the breadboard device for 20 minutes using the electric heater. A ‘chase’ buffer applied to the sample input port to dilute and move the DETECTR reaction onto the lateral flow strips. The strips were allowed to absorb the DETECTR reaction for four minutes before the results were photographed. A portion of the reaction was removed prior to incubating on the device and used as the negative control ‘off device.’ For testing Milenia HybridDetect lateral flow strips were used, which placed the control band in the lower position (13201) and the SARS-CoV-2 N-gene band distal to the control (13202).

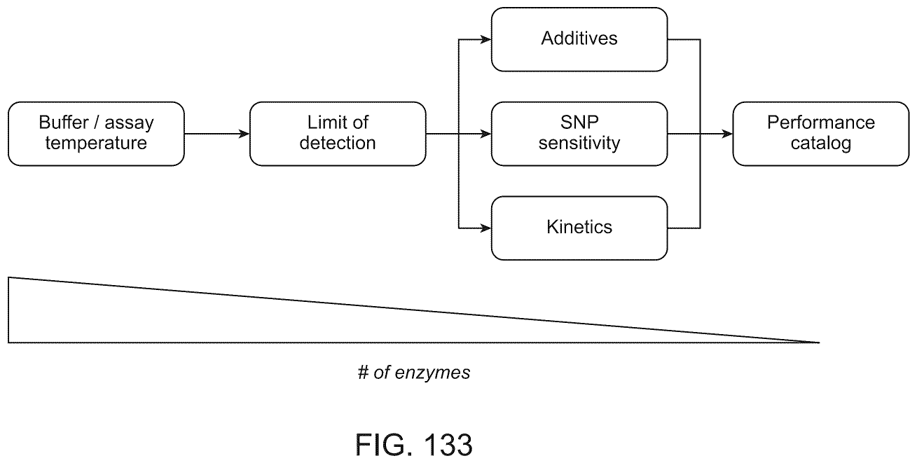

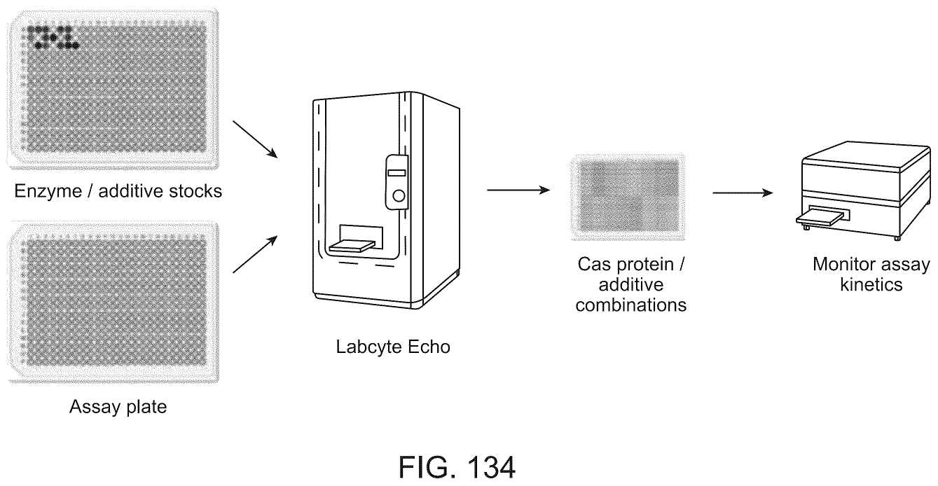

Disclosed herein are methods for optimizing proteins and formulations for diagnostic applications. An ideal Cas protein for CRISPR diagnostics is one that is fast, robust, and sensitive. A fast protein enables reduced turnaround times for diagnostic assays. Robust enzymes are more likely to be successful when combined with other molecular processes, such as in the one-pot DETECTR assay. Sensitive enzymes enable lower limits of detection from either small amounts of amplified product or when eliminating pre-amplification and doing direct detection of target nucleic acids. FIG. 133 illustrates a flow diagram of a process used to evaluate, characterize, and optimize proteins for diagnostic applications. Proteins may be evaluated based on thermostability, compatibility with various assay buffers, and sensitivity. FIG. 134 illustrates a schematic showing a workflow for the process using Labcyte Echo. Using Labcyte echo enables 4.5× faster evaluation and characterization of enzymes in comparison to manual or semi-automated setup.

Initial optimization of programmable nuclease systems (e.g., the combination of Cas protein and guide nucleic acids) involved screening the performance of candidate systems in a variety of buffers and at temperatures from 35° C. to 60° C. The buffers selected for screening included buffers used in DETECTR assays and other Cas activity buffers. The buffers may comprise a range of salt types, salt concentrations, counter ion types, and various additives important for polymerase performance.

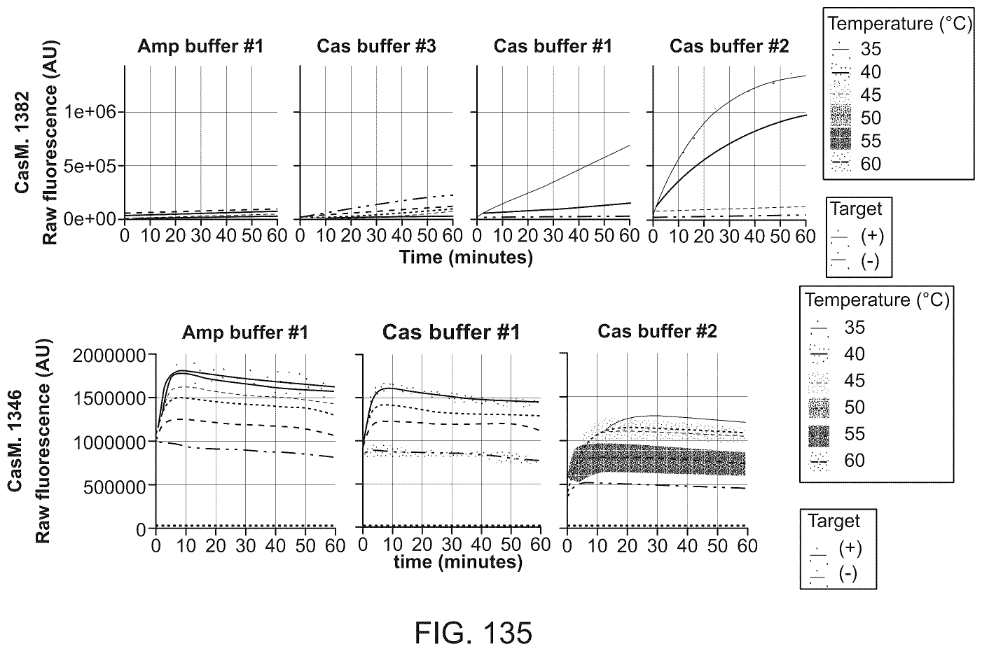

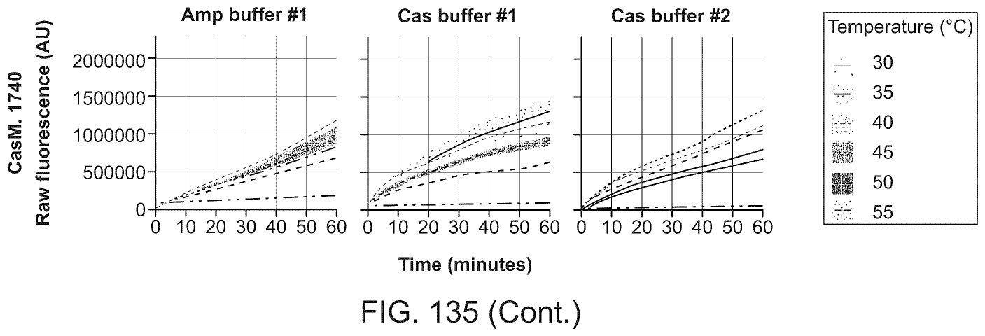

FIG. 135 shows the results of three candidate Cas enzymes trials. The results of trans-cleavage assays for the Cas enzymes exemplified the differences that were observed between enzyme performance. For example, CasM.1382 performed strongly in Cas buffer #2, and had little activity in Amp buffer #1. In Cas buffer #3, which had high levels of Mg⁺², elevated background signals were observed in the no-target controls. CasM.1346 showed strong performance in various buffers. The kinetic curves from this enzyme suggest that it was fast and sensitive, which lead to a large initial signal increase at multiple temperatures, but the enzyme quickly denatured and did not demonstrate sustained activity at temperatures over 40° C. CasM.1740 showed slower activity in comparison to CasM.1346. CasM.1740 showed robust and continued activity at temperatures as high as 50° C. but not at 55° C., suggesting that CasM. 1740 is a thermostable Cas protein and may be compatible with a variety of assay designs for direct detection or when coupled to other signal amplification processes.

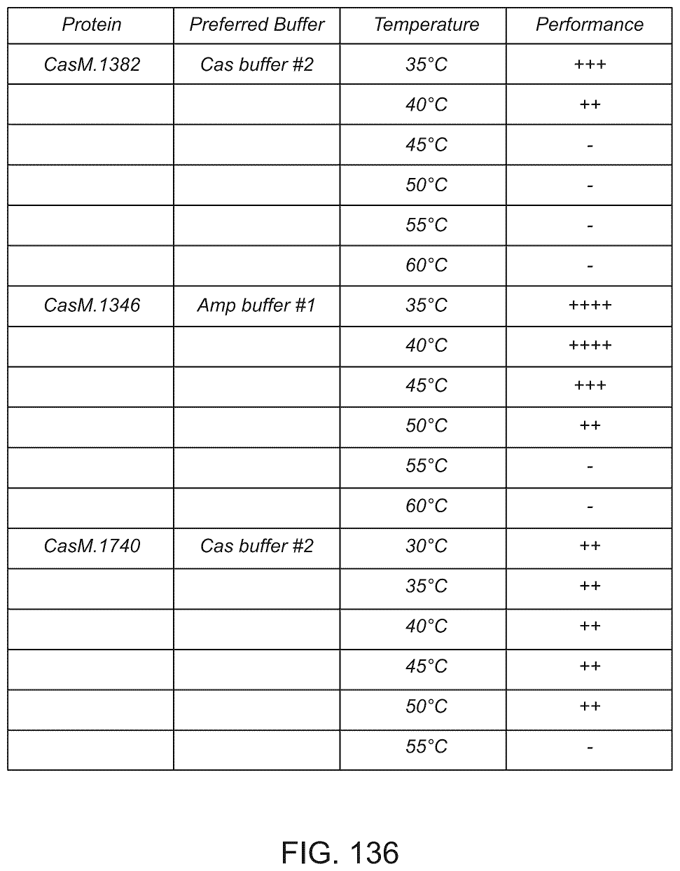

FIG. 136 shows the performance of three candidate Cas enzymes at different temperatures and buffers.

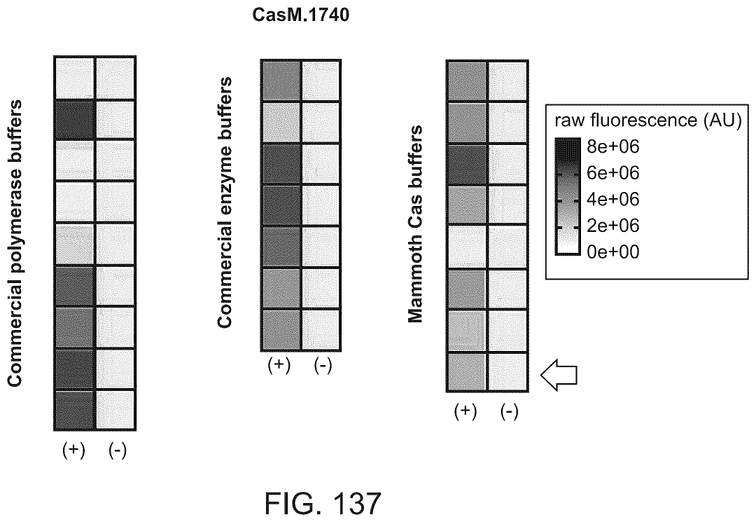

FIG. 137 shows the results of testing conducted with CasM. 1740 with three additional buffers at 35° C. CasM.1740 was robustly active in some polymerase buffers and many enzyme activity buffers. The results show that the performance of the enzymes may be optimized or enhanced by using certain buffers.

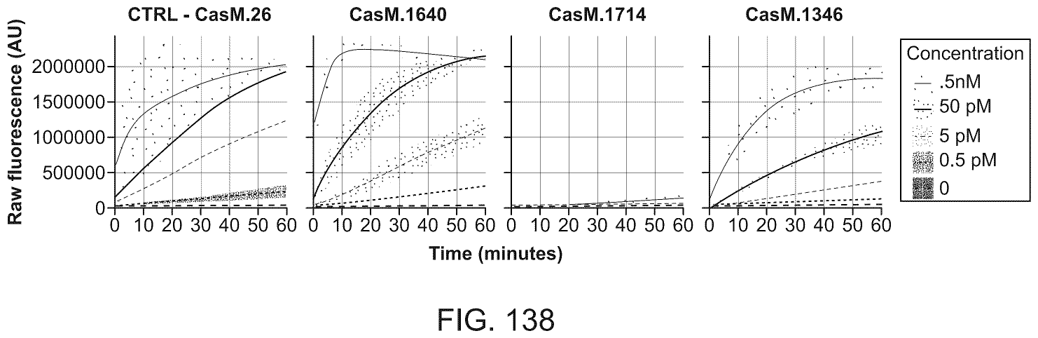

FIG. 138 shows the results of experiments investigating the limits of detection on single-strand oligo or synthetic dsDNA target at 35° C. Some systems, although functional, did not have robust limits of detection such as CasM.1714. Some performed similarly or potentially outperformed the control system CasM.26 (a Cas having SEQ ID NO: 21).

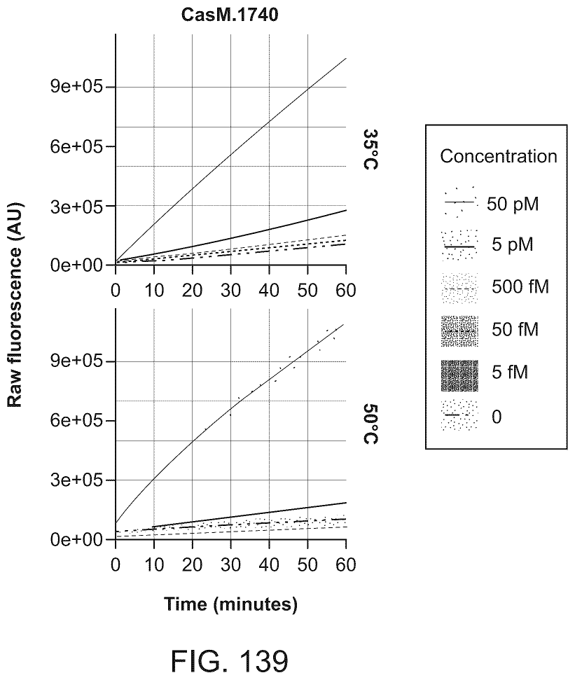

FIG. 139 shows the results of experiments evaluating the limit of detection at both 35° C. and the highest temperature that the protein was demonstrated to function at for CasM.1740. At both 35° C. and 50° C., the performance of the enzyme was similar with the ability to detect 5 μM of a target.

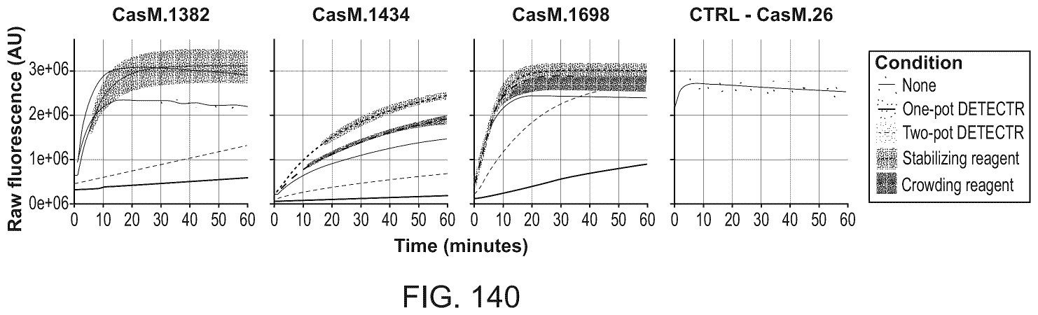

FIG. 140 shows the results of experiments investigating the effects of additives and assay formulations on the performance of the proteins. Additives included those used for lyophilization (e.g., trehalose), for stabilizing protein performance (e.g., BSA), and for reducing water content and crowding reactions (e.g., PEG). Assay formulations comprised a buffer, enzyme, primer, and dNTP concentrations used in one-pot DETECTR and two-pot DETECTR reactions. In some cases, the additives had little or no effect (e.g., CasM.1382 and CasM.1698), and in some cases, the additives boost the performance of the enzyme (e.g., CasM.1434). Some enzymes were more or less tolerant of assay formulations. For example, CasM. 1698 performed well in the two-pot DETECTR assay formulation and had reduced activity in the one-pot DETECTR assay formulation. In contrast, CasM. 1382 showed similar performance to CasM.1698 when no additives are used and was almost completely inhibited in the one-pot DETECTR assay formulation. This suggest that CasM. 1382 may be more robust in a range of assay conditions.

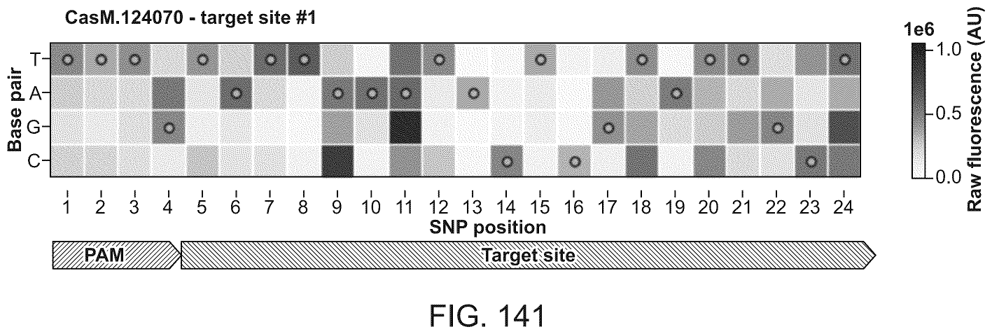

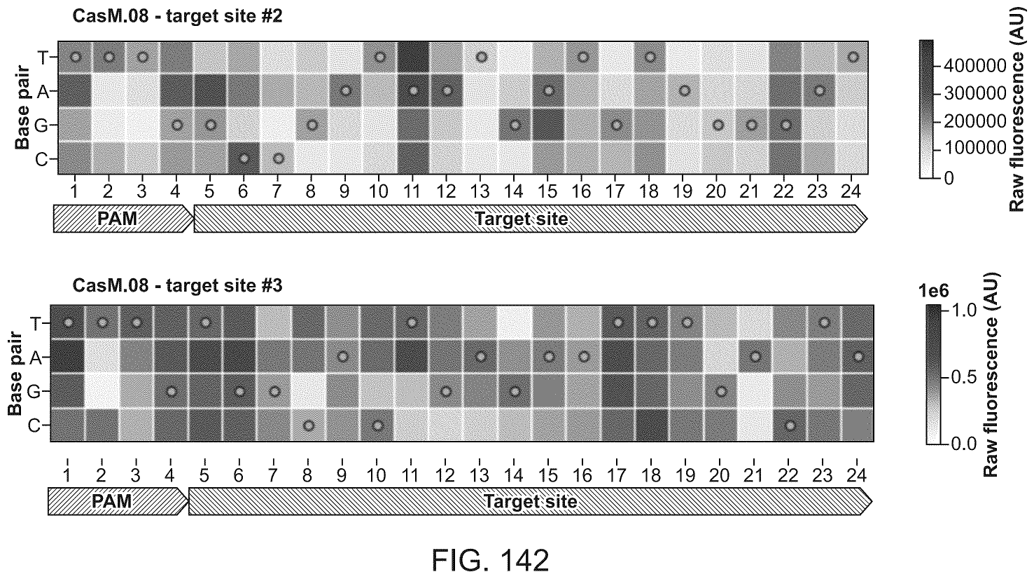

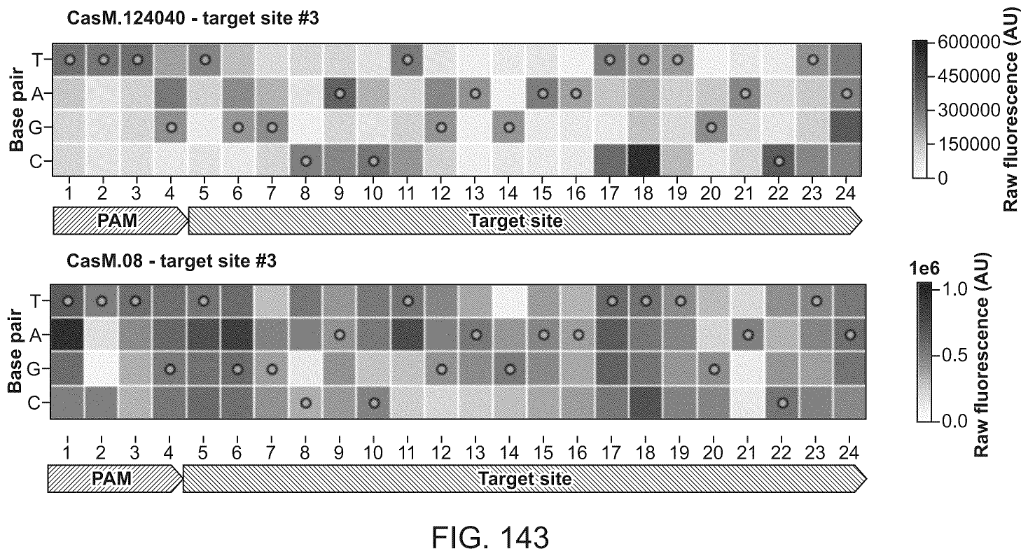

FIG. 141, FIG. 142, and FIG. 143 show the results of experiments investigating the sensitivity of Cas proteins to single-nucleotide mutations. Proteins that have high sensitivity to mutations may be useful for genotyping applications, such as the detection of viral strains or somatic genotyping for disease alleles. Meanwhile, proteins that have low sensitivity to mutations may be useful for assays where robust detection is most important. To evaluate single nucleotide mutation sensitivity of each protein, a series of synthetic dsDNA target molecules that contained all 4 potential mutations at every position along the target site and in the PAM region were synthesized. Each protein is screened in optimal conditions, as previously determined. For proteins with strong sensitivity to single nucleotide mutations, a high signal from the DETECTR assay was observed only for the nucleotides that match those in the gRNA. For proteins with low sensitivity to single nucleotide mutations, a high signal from the DETECTR assay was observed for one more nucleotides, regardless of whether the nucleotide matches those in the gRNA or not. FIG. 141 shows the results for CasM. 124070 which showed strong single nucleotide mutation sensitivity. FIG. 142 shows the results for CasM.08, which shows that the sensitivity depended on the target site. FIG. 143 shows the results for CasM.124070 and CasM.08 with the same target site, showing that different proteins had different sensitivity to the same target site.

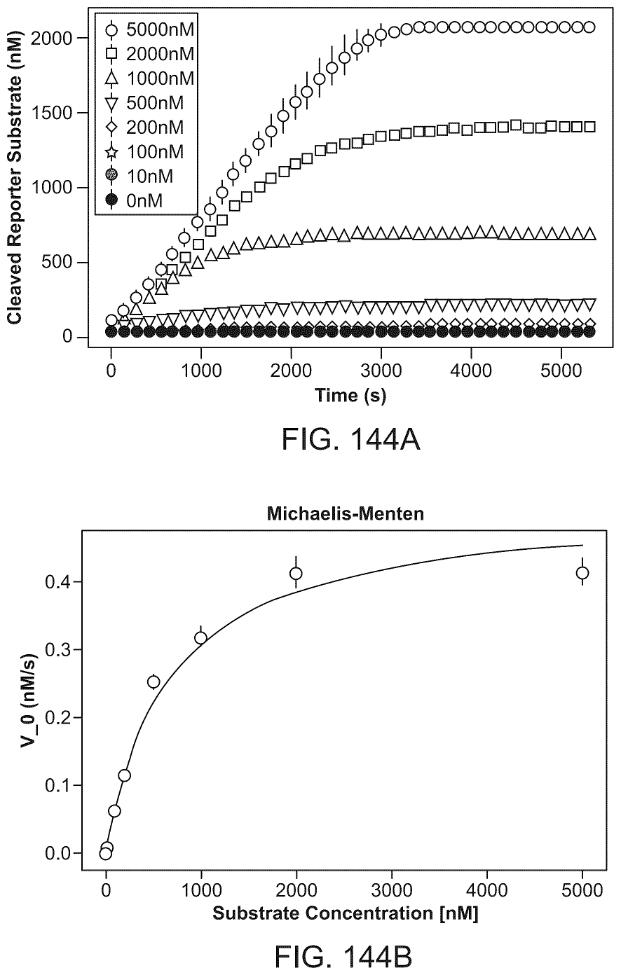

FIGS. 144A-144B show the results of experiments investigating the kinetics of trans-cleavage for proteins that have the best performance in terms of sensitivity, specificity, or thermostability. RNP complexes were prepared and incubated with target DNA or RNA for 30 minutes at the optimal temperature for the system. Various concentrations of quenched fluorescent reporters were added to the system and a plate reader was used to measure the fluorescence over time. The results were normalized across machines and temperatures for each protein. Michaelis-Menten kinetics were analyzed for each system. The results showed striking differences in the catalytic efficiency between Cas effectors. The results suggest that systems with the greatest catalytic efficiency tend to have the lowest limit of detection.

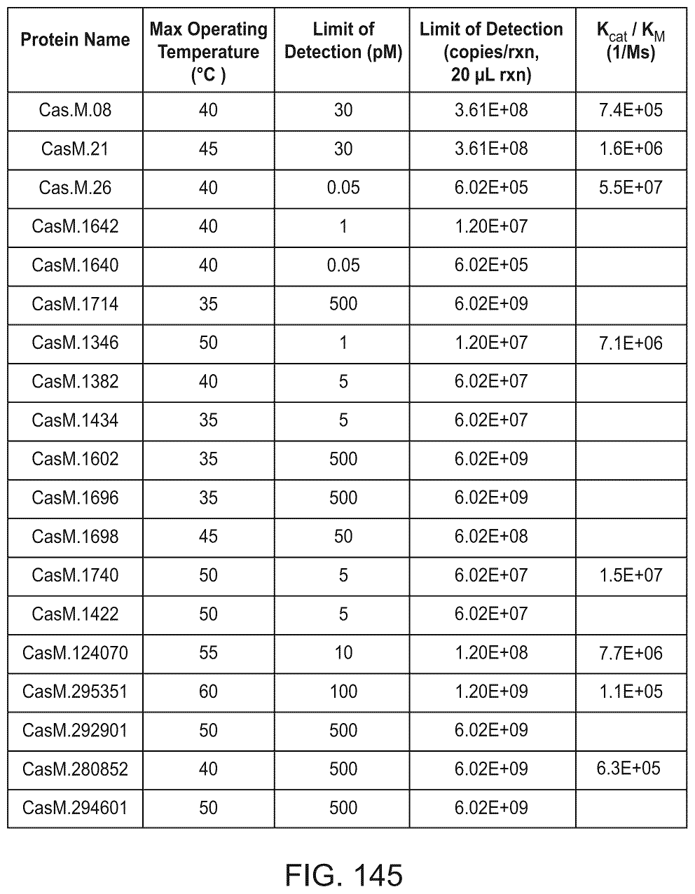

FIG. 145 summarizes the performance results for various Cas enzymes.

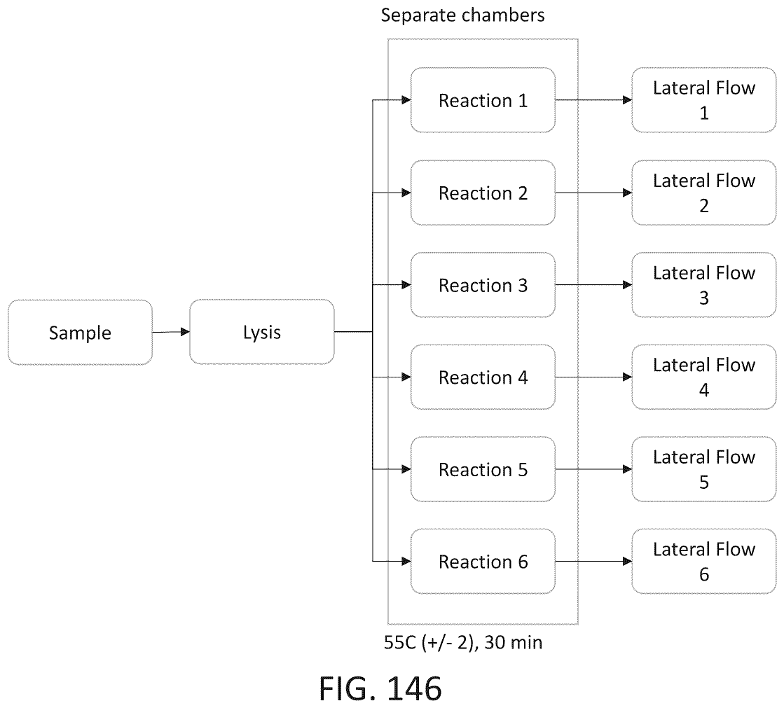

FIG. 146 shows a schematic of an exemplary workflow for a multiplexed programmable nuclease assay. Any of the devices described herein may be configured to perform one or more of the reactions described herein (e.g., amplification, detection, etc.) in separate chambers. In at least some instances, fluidly isolating reactions for different target nucleic acids may facilitate multiplexing.

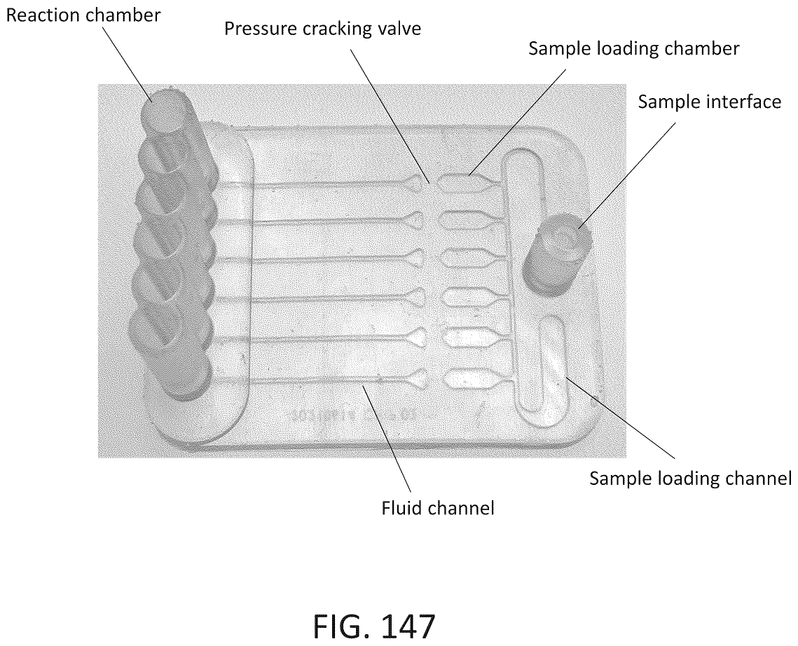

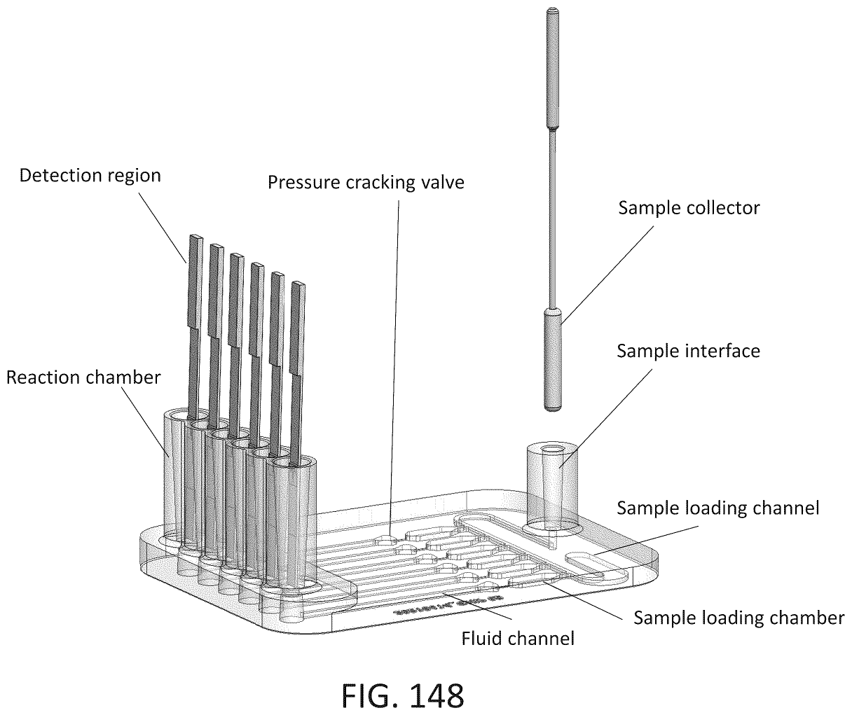

FIGS. 147 and 148 show a non-limiting exemplary handheld device for performing a multiplexed programmable nuclease (e.g., DETECTR) assay using a workflow as shown in FIG. 146. In some embodiments, a sample contains one or more target nucleic acid sequences. In some embodiments, the sample is introduced into a sample input (also referred to herein as a sample interface) and loaded into a plurality of sample loading chambers via a sample loading channel. In some embodiments, the sample interface may be configured to concentrate, filter, lyse, or otherwise prepare the sample as described herein. In some embodiments, positive pressure is applied to the sample interface by a positive pressure source (e.g., a syringe) in order to load the sample loading channel and sample loading chambers with the sample. In some embodiments, the sample is a lysis sample. The sample loading channel may be configured (e.g., sized and shaped) to load the plurality of sample loading chambers with a pre-determined volume of sample liquid. Upon loading of the sample loading chambers, sufficient positive pressure may be applied to the sample loading channel and chambers in order to break the pressure cracking valves coupled to each sample loading chamber. Sample liquid is then transferred downstream of each sample loading chamber to a reaction chamber via a fluid channel. In some embodiments, each reaction chamber is fluidly independent of every other reaction chamber in order to facilitate target analysis multiplexing. The reaction chambers may comprise one or more dried or lyophilized programmable nuclease reagents (e.g., immobilized to a glass bead) and/or one or more liquid, dried, or lyophilized amplification reagents (e.g., powdered). In some embodiments, amplification of one or more target nucleic acids is performed in each reaction chamber. In some embodiments, the amplification reagents of each reaction chamber may be different and may be designed to amplify different target nucleic acids of a plurality of target nucleic acids. The reaction chambers may be heated (e.g., to 55 C for 20 minutes or less) to amplify the one or more target nucleic acids as described herein. In some embodiments, heating may facilitate programmable nuclease-based detection reaction as described herein. Heating can include liquid-phase chemical heating, solid phase chemical heating, and electric heating (including, but not limited to, resistance/Joule heating, induction heating, Peltier heat pumping, etc.). In some embodiments, the amplification and detection reactions may be performed as a one-pot or hot pot reaction as described herein.

In some embodiments, the device includes multiple lateral flow strips. Each reaction chamber may be configured to interface with a detection region comprising a lateral flow strip. In some embodiments, the detection region may be in fluid communication with the reaction chamber. In some embodiments, the detection region may be contacted to the reaction chamber after amplification and the programmable nuclease-based reactions have been performed in the reaction chamber. In some embodiments, the lateral flow strips may be configured to be inserted into the reaction chambers as shown in FIG. 147. In some embodiments, the lateral flow strips may be configured to be inserted into the reaction chambers at the same time or at different times.

In some embodiments, each reaction chamber may comprise one or more guide nucleic acids (e.g., sgRNAs) immobilized to a surface (e.g., a glass bead or hydrogel disposed within a reaction chamber). In some embodiments, the guide nucleic acids are part of a programmable nuclease (e.g., Cas-complex) probe immobilized to a surface. In some embodiments, a guide nucleic acid is designed to specifically bind to a target nucleic acid in the sample. In some embodiments, there are different guide nucleic acids corresponding to different reaction chambers, where each different guide nucleic acid is complimentary for a different target nucleic acid sequence that may or may not be present in the sample. In some embodiments, in addition to the programmable nuclease probes containing guide nucleic acids, each reaction chamber may contain or be functionalized with one or more reporters having distinct functional groups as described herein. In some embodiments, the reporters may be in close enough proximity to be cleaved by the programmable nuclease probes. In some embodiments, as described in example 13, reporters are cleaved and portion thereof (e.g., a detection moiety) is released into the solution upon binding between a particular guide nucleic acid and the target nucleic acid to which the guide nucleic acid is designed to specifically bind. In some embodiments, reporters are functionalized with a detection moiety (e.g., a label).

In some embodiments, chemical heating may be used. In some embodiments, chemical heating may be used to supply energy to initiate and run reactions. In some embodiments, chemical heating may be used to supply energy to initiate and run programmable nuclease assay reactions. In some embodiments, chemical heating may be used to heat reaction or heating zones. In some embodiments, chemical heating may be used to heat regions, chambers, volumes, zones, surfaces, or areas of a device.

In some embodiments, the device may comprise one or more lateral flow assay strips in a detection region disposed downstream of the reaction chamber. In some embodiments, the device may comprise one or more lateral flow assay strips in a detection region which may be brought into fluid communication with the reaction chamber. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, a first lateral flow assay strip contains the capture antibody anti-FITC. In some embodiments, a first DETECTR region or surface location (e.g., within a first reaction chamber) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, upon binding of the first target nucleic acid sequence, the first immobilized reporter is cleaved and released into solution. In some embodiments, the first detection moiety is released into solution and the remainder of the first reporter remains immobilized on the surface. Alternatively, or in combination, in some embodiments, a second lateral flow assay strip contains the capture antibody anti-DIG. In some embodiments, a second DETECTR region or surface location (e.g., within a second reaction chamber) contains the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety (e.g., DIG). Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solutions containing the first or second cleaved reporters are transferred from their respective reaction chambers to a first lateral flow assay strip and a second lateral flow assay strip, respectively. In some embodiments, a chase buffer or diluent is introduced into a diluent input and negative pressure is applied to the negative pressure port to contact the solutions containing the first or second cleaved reporters to their respective lateral flow assay strips. The reporters may be bound to conjugate molecules e.g., anti-biotin-AuNPs. In some embodiments, the AuNP-reporter conjugates having the first reporter labeled with the first detection moiety (e.g., FITC) will selectively bind to a first detection region or spot containing the first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing the second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the device may comprise more than one lateral flow assay strip. For example, the device may comprise two, three, four, five, six, seven, eight, nine, ten, or more lateral flow assay strips. In some embodiments, each reaction chamber may be interfaced with a lateral flow assay strip for multiplexing. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

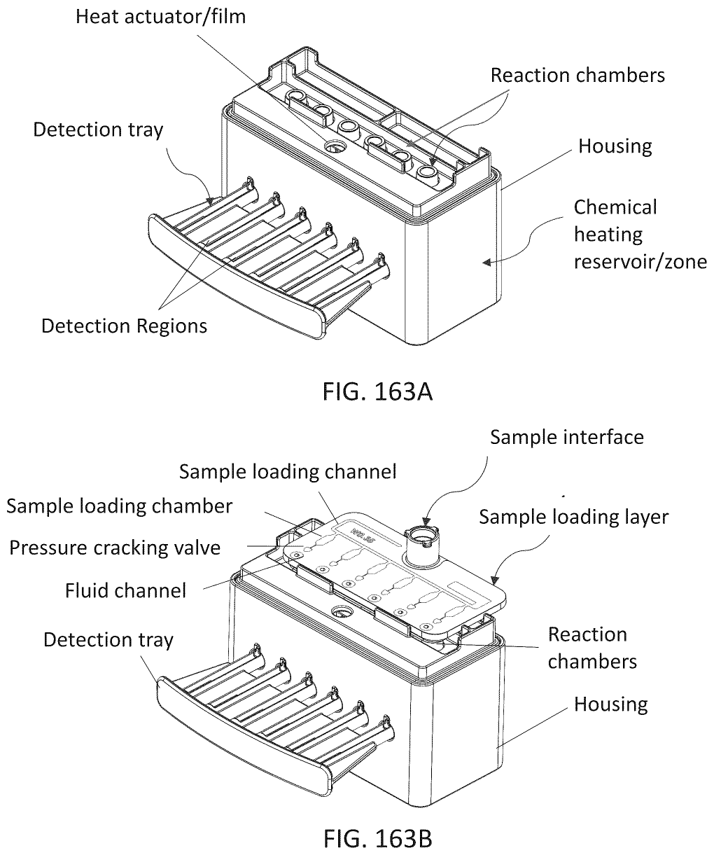

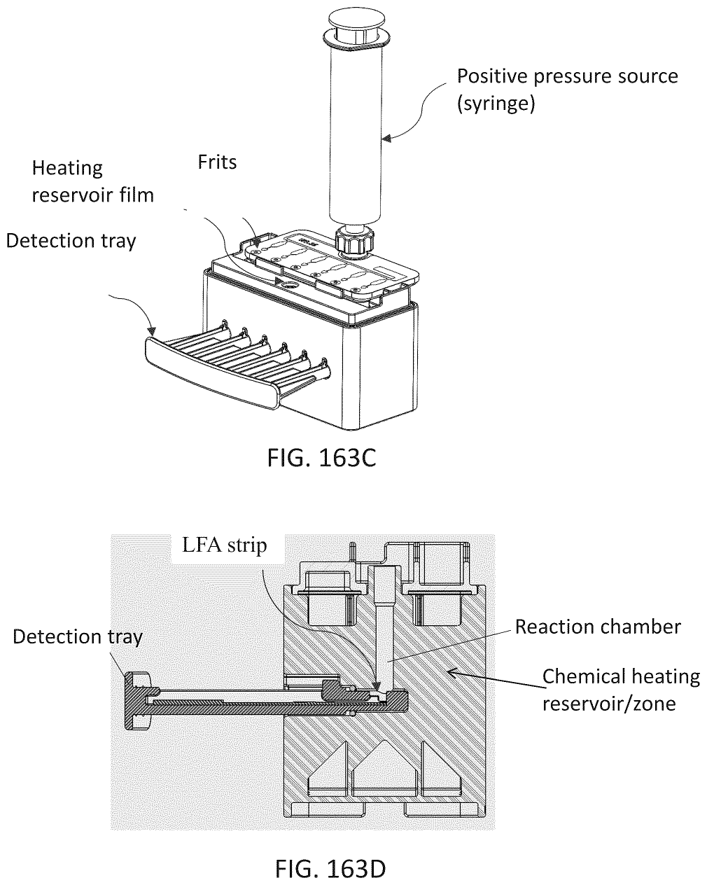

Described herein are various methods and devices for chemical heating-based PON devices utilizing lateral flow assay strips for DETECTR readout. FIGS. 163A-163D illustrate a non-limiting example of a handheld device for performing a multiplexed programmable nuclease (e.g., DETECTR) assay using a workflow as shown in FIG. 146. In some embodiments, the handheld device may be configured to provide a heating source for applications (e.g., amplification and/or DETECTR reactions) that do not allow for the use of battery or other electrical power supplies (such as in remote locations without easy access to reliable sources of electricity. In certain instances, the handheld device may comprise sodium acetate to provide heating to one or more heating regions of the device, via heat released by the exothermic chemical reaction of sodium acetate. In some embodiments, the design of the handheld device may allow for uniform heating of the amplification and/or DETECTR reaction components by uniformly surrounding the reaction chamber(s) with a sodium acetate heating agent reservoir.

FIGS. 163A-163D illustrate a non-limiting example of a handheld device for performing a multiplexed programmable nuclease (e.g., DETECTR) assay using a workflow as shown in FIG. 146. FIG. 163A shows a perspective view of the handheld device with reaction chambers exposed. FIG. 163B shows a perspective view of the handheld device with a sample loading layer covering the reaction chambers. FIG. 163C shows a perspective view of the fully assembled handheld device including positive pressure source engaged with a sample interface of the sample loading layer. FIG. 163D shows a cross-sectional view of the handheld device highlighting the relative locations of the reaction chamber and detection region within a heating reservoir. The device of FIGS. 163A-163D may be substantially similar to the devices shown in FIGS. 147-148, with the addition of a heating reservoir configured to surround the reaction chambers in order to provide integrated, sustained, even heating during isothermal amplification and/or programmable nuclease-based detection reactions.

In some embodiments, a sample contains one or more target nucleic acid sequences. In some embodiments, the sample is introduced into a sample input (also referred to herein as a sample interface) and loaded into a plurality of sample loading chambers via a sample loading channel. In some embodiments, the sample interface may be configured to concentrate, filter, lyse, or otherwise prepare the sample as described herein. In some embodiments, the sample may be loaded into the sample loading channel and sample loading chambers automatically via gravity and/or capillary forces. Alternatively, or in combination, positive pressure may be applied to the sample interface by a positive pressure source (e.g., a syringe) in order to load the sample loading channel and sample loading chambers with the sample. In some embodiments, the sample is a lysis sample. The sample loading channel may be configured (e.g., sized and shaped) to load the plurality of sample loading chambers with a pre-determined volume of sample liquid. Upon loading of the sample loading chambers, sufficient positive pressure may be applied to the sample loading channel and chambers in order to break the pressure cracking valves coupled to each sample loading chamber. Sample liquid is then transferred downstream of each sample loading chamber to a reaction chamber via a fluid channel. In some embodiments, a downstream end of the fluid channel may comprise a valve, hydrophobic filter, or frit or the like configured to allow gas to pass through during positive pressure application to the sample interface and enable fluid to flow along the fluid channel unimpeded. The hydrophobic filter or frit may be configured to prevent liquid to pass through as the sample is moved into the reaction chamber. In some embodiments, the sample input, sample loading channel(s), and/or sample loading chamber(s) may be located in a sample loading layer of the device. In some embodiments, the sample loading layer may be fluidly coupled to the reaction chambers. In some embodiments, the sample loading layer may be disposed above the reaction chambers. In some embodiments, the sample loading layer may be a chip or microfluidic device assembled over a housing comprising the reaction chambers. In some embodiments, each reaction chamber is fluidly independent of every other reaction chamber in order to facilitate target analysis multiplexing. The reaction chambers may comprise one or more dried or lyophilized programmable nuclease reagents (e.g., immobilized to a glass bead) and/or one or more liquid, dried, or lyophilized amplification reagents (e.g., powdered). In some embodiments, the programmable nuclease reagents and/or amplification reagents may be added by a user prior to use (e.g., pipetted into the reaction chambers by a user prior to connecting a sample loading layer thereto) and/or added during manufacturing. In some embodiments, amplification of one or more target nucleic acids is performed in each reaction chamber. In some embodiments, the amplification reagents of each reaction chamber may be different and may be designed to amplify different target nucleic acids of a plurality of target nucleic acids. The reaction chambers may be heated (e.g., to 55 C for 20 minutes or less) to amplify the one or more target nucleic acids as described herein. In some embodiments, heating may facilitate programmable nuclease-based detection reaction as described herein. Heating can include liquid-phase chemical heating, solid phase chemical heating, and electric heating (including, but not limited to, resistance/Joule heating, induction heating, Peltier heat pumping, etc.). In some embodiments, the reaction chambers may be surrounded by or disposed in a heating reservoir or zone. For example, the reaction chambers may comprise vertical chambers which are substantially surrounded by a heating reservoir as shown in FIG. 163D. The heating reservoir may comprise a liquid-phase chemical heating agent, a solid-phase chemical heating agent, or an electrical heater. In some embodiments, the amplification and detection reactions may be performed as a one-pot or hot pot reaction as described herein.

In some embodiments, the device includes multiple lateral flow strips. Each reaction chamber may be configured to interface with a detection region comprising a lateral flow strip. In some embodiments, the detection region may be in fluid communication with the reaction chamber. In some embodiments, the detection region may be contacted to the reaction chamber after amplification and the programmable nuclease-based reactions have been performed in the reaction chamber. In some embodiments, the lateral flow strips may be configured to be inserted into the reaction chambers as shown in FIG. 163D. In some embodiments, the lateral flow strips may be configured to be inserted into the reaction chambers at the same time or at different times. For example, the lateral flow strips may be disposed on a detection tray configured to be actuated from a first position, in which the lateral flow strips are not in fluid communication with their respective reaction chambers, to a second position, in which the lateral flow strips are in fluid communication with their respective reaction chambers. In some embodiments, the reaction chambers may be vertical chambers surrounded by a heating reservoir and the detection tray may be configured to hold the lateral flow strips at an angle relative to the vertical chambers (e.g., perpendicular) in order to facilitate wicking and capillary flow of the fluid from the reaction chambers along the lateral flow strips. Insertion of the detection tray into the housing comprising the chemical heating reservoir and reaction chambers may actuate the detection tray from the first position to the section position and bring the lateral flow strips into fluid communication with their respective reaction chambers in order to enable visual readout of the programmable nuclease-based reaction.

In some embodiments, each reaction chamber may comprise one or more guide nucleic acids (e.g., sgRNAs) immobilized to a surface (e.g., a glass bead or hydrogel disposed within a reaction chamber). In some embodiments, the guide nucleic acids are part of a programmable nuclease (e.g., Cas-complex) probe immobilized to a surface. In some embodiments, a guide nucleic acid is designed to specifically bind to a target nucleic acid in the sample. In some embodiments, there are different guide nucleic acids corresponding to different reaction chambers, where each different guide nucleic acid is complimentary for a different target nucleic acid sequence that may or may not be present in the sample. In some embodiments, in addition to the programmable nuclease probes containing guide nucleic acids, each reaction chamber may contain or be functionalized with one or more reporters having distinct functional groups as described herein. In some embodiments, the reporters may be in close enough proximity to be cleaved by the programmable nuclease probes. In some embodiments, as described in example 13, reporters are cleaved and portion thereof (e.g., a detection moiety) is released into the solution upon binding between a particular guide nucleic acid and the target nucleic acid to which the guide nucleic acid is designed to specifically bind. In some embodiments, reporters are functionalized with a detection moiety (e.g., a label).

In some embodiments, chemical heating may be used. In some embodiments, chemical heating may be used to supply energy to initiate and run reactions. In some embodiments, chemical heating may be used to supply energy to initiate and run programmable nuclease assay reactions. In some embodiments, chemical heating may be used to heat reaction or heating zones. In some embodiments, chemical heating may be used to heat regions, chambers, volumes, zones, surfaces, or areas of a device. In some embodiments, the reaction chambers may be surrounded by or disposed in a heating reservoir or zone. The heating reservoir may comprise a liquid-phase chemical heating agent configured to generate an exothermic chemical reaction upon contact with a nucleation site. For example, a piercer may be configured to pierce through a film surrounding the heating reservoir and contact the liquid-phase chemical heating agent (e.g., sodium acetate). The piercer may provide a nucleation site for crystallization of the liquid-phase chemical heating agent to begin phase change transformation and thereby generate heat within the heating reservoir. The amount of chemical heating agent may be adjusted to provide a desired temperature and/or duration of heating to the reaction chambers.

In some embodiments, the device may comprise one or more lateral flow assay strips in a detection region disposed downstream of the reaction chamber. In some embodiments, the device may comprise one or more lateral flow assay strips in a detection region which may be brought into fluid communication with the reaction chamber. In some embodiments, the detection region comprises a detection tray. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, a first lateral flow assay strip contains the capture antibody anti-FITC. In some embodiments, a first DETECTR region or surface location (e.g., within a first reaction chamber) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, the first reporter may be immobilized within a hydrogel as described herein. In some embodiments, upon binding of the first target nucleic acid sequence, the first immobilized reporter is cleaved and released into solution. In some embodiments, the first detection moiety is released into solution and the remainder of the first reporter remains immobilized on the surface. Alternatively, or in combination, in some embodiments, a second lateral flow assay strip contains the capture antibody anti-DIG. In some embodiments, a second DETECTR region or surface location (e.g., within a second reaction chamber) contains the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety (e.g., DIG). In some embodiments, the second reporter may be immobilized within a hydrogel as described herein. Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solutions containing the first or second cleaved reporters are transferred from their respective reaction chambers to a first lateral flow assay strip and a second lateral flow assay strip, respectively. In some embodiments, a chase buffer or diluent is introduced into a diluent input and pressure is applied to contact the solutions containing the first or second cleaved reporters to their respective lateral flow assay strips. In some embodiments, the reaction chambers are vertical reaction chambers and gravity is used to provide fluid flow between the reaction chambers and their respective lateral flow assay strips. The reporters may be bound to conjugate molecules e.g., anti-biotin-AuNPs. In some embodiments, the AuNP-reporter conjugates having the first reporter labeled with the first detection moiety (e.g., FITC) will selectively bind to a first detection region or spot containing the first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing the second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the device may comprise more than one lateral flow assay strip. For example, the device may comprise two, three, four, five, six, seven, eight, nine, ten, or more lateral flow assay strips. In some embodiments, each reaction chamber may be interfaced with a lateral flow assay strip for multiplexing. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

In some embodiments, the isothermal amplification and/or programmable nuclease-based reaction may be completed in about 40 minutes. In some embodiments, the isothermal amplification and/or programmable nuclease-based reaction may be completed in less than about 40 minutes. In some embodiments, the isothermal amplification and/or programmable nuclease-based reaction may be completed in greater than about 40 minutes. In some embodiments, the isothermal amplification and/or programmable nuclease-based reaction may be completed in about 25 minutes to about 35 minutes. In some embodiments, the isothermal amplification and/or programmable nuclease-based reaction may be completed in about 35 minutes to about 45 minutes. In some embodiments, the isothermal amplification and/or programmable nuclease-based reaction may be completed in about 45 minutes to about 55 minutes.

In some embodiments, results may be ready in less than about 1 minute after the sample begins the capillary process across the lateral flow assay strip. In some embodiments, results may be ready in less than about 2 minutes after the sample begins the capillary process across the lateral flow assay strip. In some embodiments, results may be ready in less than about 3 minutes after the sample begins the capillary process across the lateral flow assay strip. In some embodiments, results may be ready in less than about 5 minutes after the sample begins the capillary process across the lateral flow assay strip.

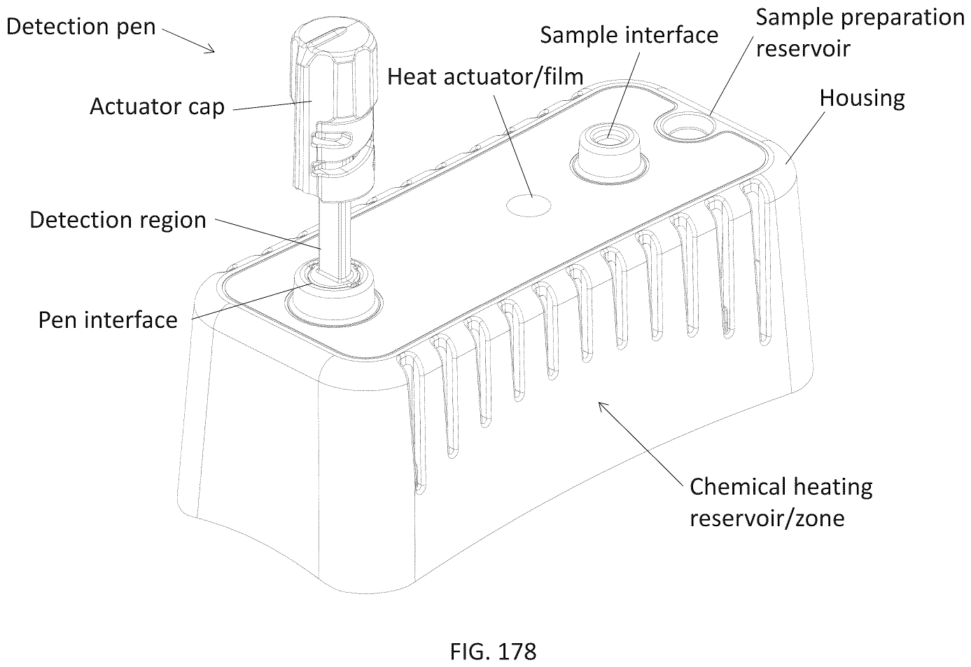

Described herein are various methods and devices for chemical heating-based PON devices utilizing lateral flow assay strips for DETECTR readout. FIGS. 178-184 illustrate a non-limiting example of a handheld device for performing a programmable nuclease (e.g., DETECTR) assay using a workflow as shown in FIG. 146. In some embodiments, the handheld device may be configured to provide a heating source for applications (e.g., amplification and/or DETECTR reactions) that do not allow for the use of battery or other electrical power supplies (such as in remote locations without easy access to reliable sources of electricity. In certain instances, the handheld device may comprise sodium acetate to provide heating to one or more heating regions of the device, via heat released by the exothermic chemical reaction of sodium acetate. In some embodiments, the design of the handheld device may allow for uniform heating of the amplification and/or DETECTR reaction components by uniformly surrounding the reaction chamber(s) with a sodium acetate heating agent reservoir.

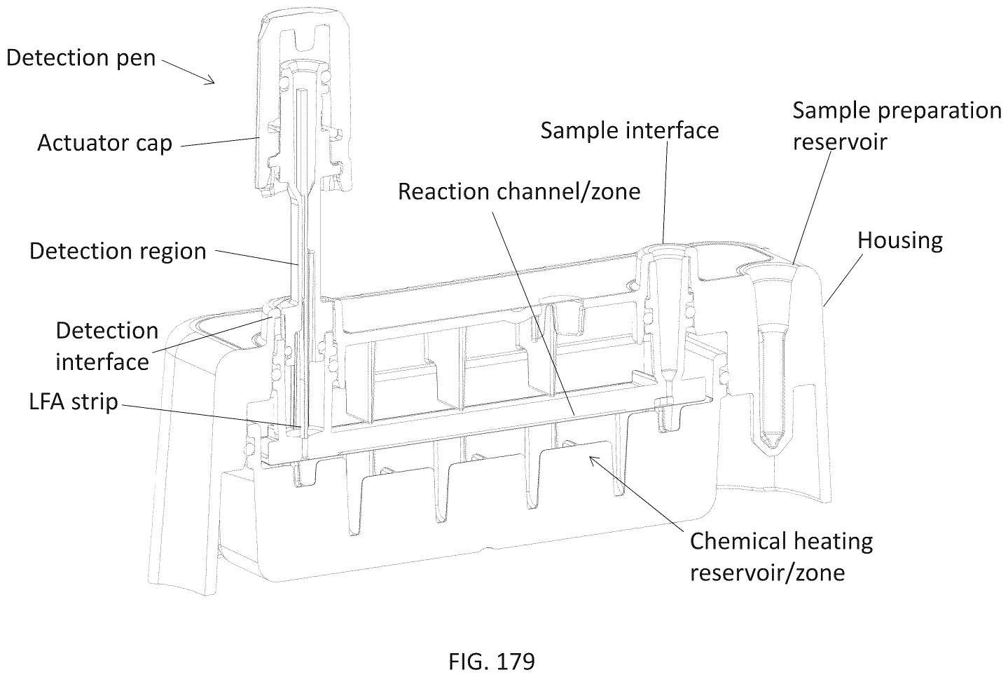

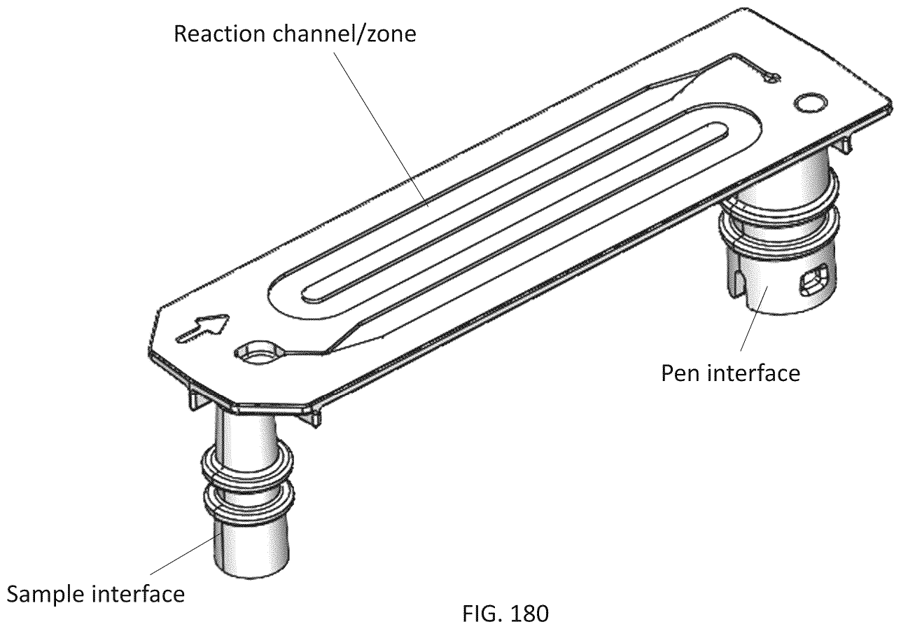

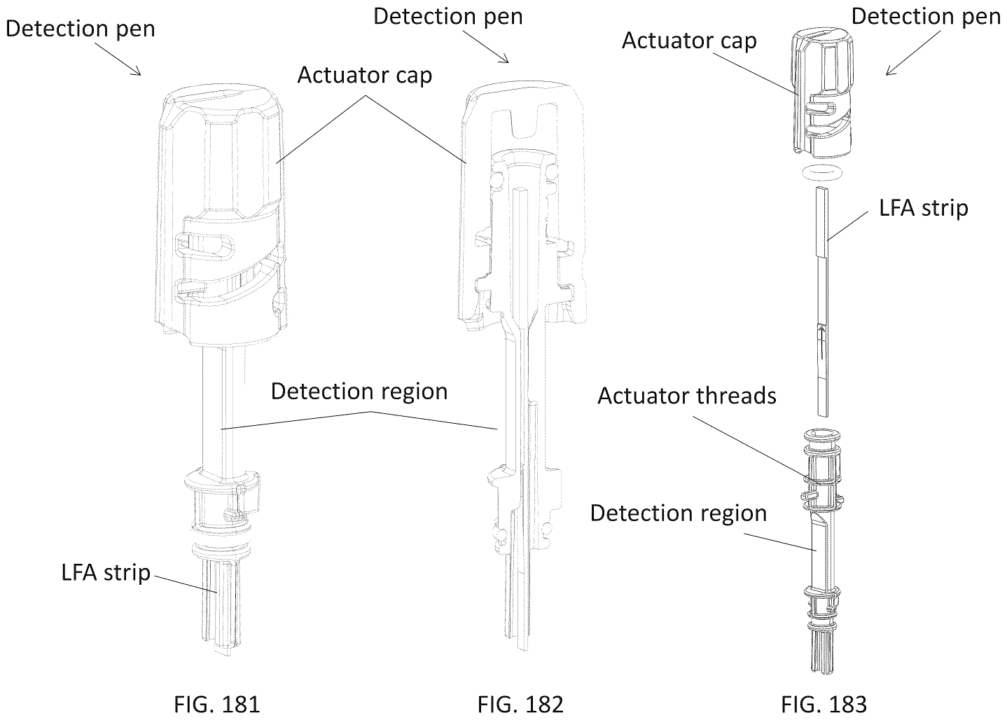

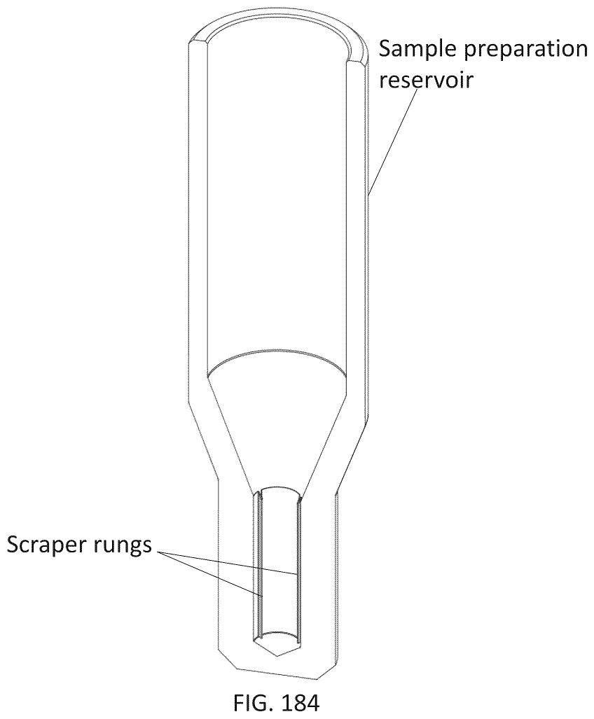

FIGS. 178-184 illustrate a non-limiting example of a handheld device for performing a programmable nuclease (e.g., DETECTR) assay using a workflow as shown in FIG. 146. FIG. 178 shows a perspective view of the fully assembled handheld device including negative pressure source (e.g., detection pen) engaged with a detection interface of the housing. FIG. 179 shows a cross-sectional view of the handheld device highlighting the relative locations of the reaction channel/chamber and detection region within a heating reservoir. FIG. 180 shows a reaction channel component of the handheld device. FIG. 181 shows an isometric view of a detection pen of the handheld device. FIG. 182 shows a cross-sectional view of the detection pen. FIG. 183 shows an exploded view of the detection pen. FIG. 184 shows a cross-sectional view of a sample preparation reservoir of the handheld device. The handheld device of FIGS. 163A-163D may be substantially similar to the devices shown in FIGS. 163A-163D, with the addition of a detection pen configured to provide negative pressure to the system to affect fluid movement therein and provide a fluid pathway for the reaction volume to contact a lateral flow assay strip disposed therein. Additionally, an optional sample preparation reservoir comprising a scraper comprising a plurality of vertical rungs may be provided by the handheld device.

In some embodiments, a sample contains one or more target nucleic acid sequences. In some embodiments, the sample is introduced into a sample input (also referred to herein as a sample interface) and loaded into a plurality of sample loading chambers via a sample loading channel. In some embodiments, the sample interface may be configured to concentrate, filter, lyse, or otherwise prepare the sample as described herein. In some embodiments, a separate sample preparation reservoir may be provided and configured to concentrate, filter, lyse, or otherwise prepare the sample as described herein before the sample is loaded into the sample interface. In some embodiments, the sample may be loaded into the sample loading channel and sample loading chambers automatically via gravity and/or capillary forces. Alternatively, or in combination, negative pressure may be applied to the detection interface by a negative pressure source (e.g., a syringe) in order to load the sample loading channel and sample loading chambers with the sample. In some embodiments, the negative pressure source may be in the form of a detection pen comprising a lateral flow assay strip disposed therein. Counter-clockwise, upwards rotation of the actuator cap of the detection pen from a first configuration to a second configuration may draw fluid into the reaction channel or chamber from the sample interface. In some embodiments, the sample is a lysis sample. In some embodiments, the device may comprise a single reaction channel or chamber. In some embodiments, the device may comprise a plurality of reaction channels or chambers. In some embodiments, each reaction chamber is fluidly independent of every other reaction chamber in order to facilitate target analysis multiplexing. The reaction chamber(s) may comprise one or more dried or lyophilized programmable nuclease reagents (e.g., immobilized to a glass bead) and/or one or more liquid, dried, or lyophilized amplification reagents (e.g., powdered). In some embodiments, the programmable nuclease reagents and/or amplification reagents may be added by a user prior to use and/or added during manufacturing. In some embodiments, amplification of one or more target nucleic acids is performed in each reaction chamber. In some embodiments, the amplification reagents of each reaction chamber may be different and may be designed to amplify different target nucleic acids of a plurality of target nucleic acids. The reaction chambers may be heated (e.g., to 55 C for 20 minutes or less) to amplify the one or more target nucleic acids as described herein. In some embodiments, heating may facilitate programmable nuclease-based detection reaction as described herein. Heating can include liquid-phase chemical heating, solid phase chemical heating, and electric heating (including, but not limited to, resistance/Joule heating, induction heating, Peltier heat pumping, etc.). In some embodiments, the reaction chambers may be surrounded by or disposed in a heating reservoir or zone. For example, the reaction chambers may comprise serpentine channels or spiral tubes which are substantially surrounded by a heating reservoir as shown in FIG. 180. The heating reservoir may comprise a liquid-phase chemical heating agent, a solid-phase chemical heating agent, or an electrical heater. In some embodiments, the amplification and detection reactions may be performed as a one-pot or hot pot reaction as described herein.

In some embodiments, the device includes one or more lateral flow strips disposed within one or more detection pens. Each reaction chamber may be configured to interface with a detection region comprising a lateral flow strip. In some embodiments, the detection region may be in fluid communication with the reaction chamber. In some embodiments, the detection region may be contacted to the reaction chamber after amplification and the programmable nuclease-based reactions have been performed in the reaction chamber. In some embodiments, the lateral flow strip may be configured to be inserted adjacent or into the reaction chamber/channel as shown in FIG. 179. In some embodiments, a plurality of lateral flow strips may be configured to be inserted into the reaction chambers at the same time or at different times. In some embodiments, the lateral flow strip(s) may be disposed in a detection pen(s) configured to be actuated from a first position, in which the lateral flow strip(s) is not in fluid communication with its respective reaction chamber(s), to a second position, in which the lateral flow strip(s) is in fluid communication with its respective reaction chamber(s). In some embodiments, the lateral flow strip(s) may be disposed in a detection pen(s) configured to draw the reaction liquid along a fluid flow path(s) toward the lateral flow strip(s). The lateral flow strip(s) may be in fluid communication with the reaction chamber(s) and actuation of the actuator cap of the detection pen from the second configuration to a third configuration may provide negative pressure and draw the reaction products from the reaction chamber to the lateral flow strips. In some embodiments, the detection pen may be configured to hold the lateral flow strips at an angle relative to the reaction chambers (e.g., perpendicular, vertical).

In some embodiments, each reaction chamber may comprise one or more guide nucleic acids (e.g., sgRNAs) immobilized to a surface (e.g., a glass bead or hydrogel disposed within a reaction chamber). In some embodiments, the guide nucleic acids are part of a programmable nuclease (e.g., Cas-complex) probe immobilized to a surface. In some embodiments, a guide nucleic acid is designed to specifically bind to a target nucleic acid in the sample. In some embodiments, there are different guide nucleic acids corresponding to different reaction chambers, where each different guide nucleic acid is complimentary for a different target nucleic acid sequence that may or may not be present in the sample. In some embodiments, in addition to the programmable nuclease probes containing guide nucleic acids, each reaction chamber may contain or be functionalized with one or more reporters having distinct functional groups as described herein. In some embodiments, the reporters may be in close enough proximity to be cleaved by the programmable nuclease probes. In some embodiments, as described in example 13, reporters are cleaved and portion thereof (e.g., a detection moiety) is released into the solution upon binding between a particular guide nucleic acid and the target nucleic acid to which the guide nucleic acid is designed to specifically bind. In some embodiments, reporters are functionalized with a detection moiety (e.g., a label). In some embodiments, chemical heating may be used. In some embodiments, chemical heating may be used to supply energy to initiate and run reactions. In some embodiments, chemical heating may be used to supply energy to initiate and run programmable nuclease assay reactions. In some embodiments, chemical heating may be used to heat reaction or heating zones. In some embodiments, chemical heating may be used to heat regions, chambers, volumes, zones, surfaces, or areas of a device. In some embodiments, the reaction chambers may be surrounded by or disposed in a heating reservoir or zone. The heating reservoir may comprise a liquid-phase chemical heating agent configured to generate an exothermic chemical reaction upon contact with a nucleation site. For example, a piercer may be configured to pierce through a film surrounding the heating reservoir and contact the liquid-phase chemical heating agent (e.g., sodium acetate). The piercer may provide a nucleation site for crystallization of the liquid-phase chemical heating agent to begin phase change transformation and thereby generate heat within the heating reservoir. The amount of chemical heating agent may be adjusted to provide a desired temperature and/or duration of heating to the reaction chambers.

In some embodiments, the device may comprise one or more lateral flow assay strips in a detection region disposed downstream of the reaction chamber. In some embodiments, the device may comprise one or more lateral flow assay strips in a detection region which may be brought into fluid communication with the reaction chamber. In some embodiments, the detection region comprises a detection tray. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, a first lateral flow assay strip contains the capture antibody anti-FITC. In some embodiments, a first DETECTR region or surface location (e.g., within a first reaction chamber) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, the first reporter may be immobilized within a hydrogel as described herein. In some embodiments, upon binding of the first target nucleic acid sequence, the first immobilized reporter is cleaved and released into solution. In some embodiments, the first detection moiety is released into solution and the remainder of the first reporter remains immobilized on the surface. Alternatively, or in combination, in some embodiments, a second lateral flow assay strip contains the capture antibody anti-DIG. In some embodiments, a second DETECTR region or surface location (e.g., within a second reaction chamber) contains the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety (e.g., DIG). In some embodiments, the second reporter may be immobilized within a hydrogel as described herein. Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solutions containing the first or second cleaved reporters are transferred from their respective reaction chambers to a first lateral flow assay strip and a second lateral flow assay strip, respectively. In some embodiments, a chase buffer or diluent is introduced into a diluent input and pressure is applied to contact the solutions containing the first or second cleaved reporters to their respective lateral flow assay strips. In some embodiments, the reaction chambers are vertical reaction chambers and gravity is used to provide fluid flow between the reaction chambers and their respective lateral flow assay strips. The reporters may be bound to conjugate molecules e.g., anti-biotin-AuNPs. In some embodiments, the AuNP-reporter conjugates having the first reporter labeled with the first detection moiety (e.g., FITC) will selectively bind to a first detection region or spot containing the first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing the second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the device may comprise more than one lateral flow assay strip. For example, the device may comprise two, three, four, five, six, seven, eight, nine, ten, or more lateral flow assay strips. In some embodiments, each reaction chamber may be interfaced with a lateral flow assay strip for multiplexing. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

In some embodiments, the isothermal amplification and/or programmable nuclease-based reaction may be completed in about 40 minutes. In some embodiments, the isothermal amplification and/or programmable nuclease-based reaction may be completed in less than about 40 minutes. In some embodiments, the isothermal amplification and/or programmable nuclease-based reaction may be completed in greater than about 40 minutes. In some embodiments, the isothermal amplification and/or programmable nuclease-based reaction may be completed in about 25 minutes to about 35 minutes. In some embodiments, the isothermal amplification and/or programmable nuclease-based reaction may be completed in about 35 minutes to about 45 minutes. In some embodiments, the isothermal amplification and/or programmable nuclease-based reaction may be completed in about 45 minutes to about 55 minutes.

In some embodiments, results may be ready in less than about 1 minute after the sample begins the capillary process across the lateral flow assay strip. In some embodiments, results may be ready in less than about 2 minutes after the sample begins the capillary process across the lateral flow assay strip. In some embodiments, results may be ready in less than about 3 minutes after the sample begins the capillary process across the lateral flow assay strip. In some embodiments, results may be ready in less than about 5 minutes after the sample begins the capillary process across the lateral flow assay strip.

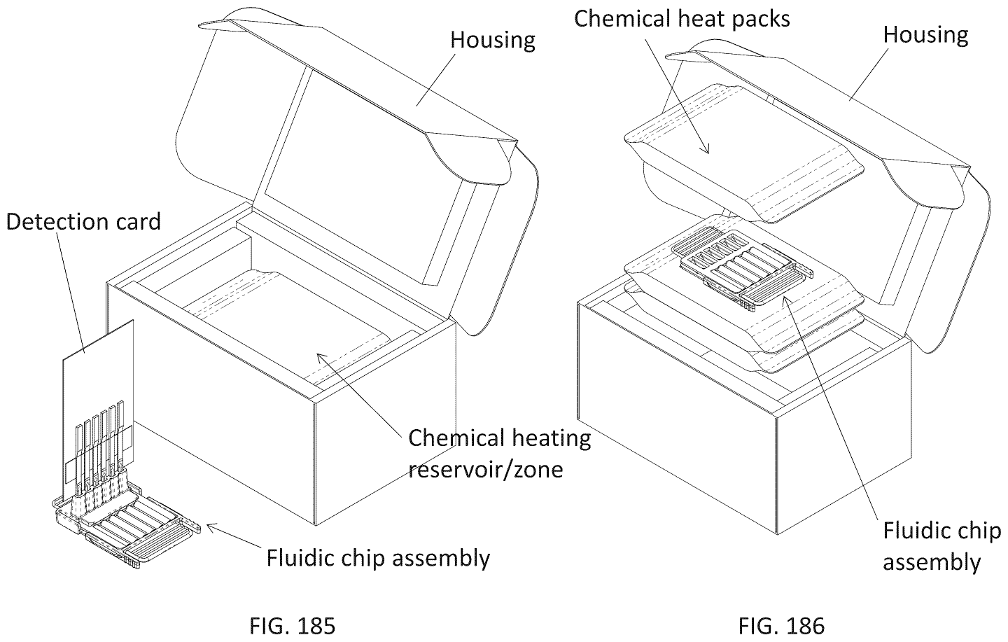

Described herein are various methods and devices for chemical heating-based PON devices utilizing lateral flow assay strips for DETECTR readout. FIGS. 185-191C illustrate a non-limiting example of a handheld device for performing a multiplexed programmable nuclease (e.g., DETECTR) assay using a workflow as shown in FIG. 146. In some embodiments, the handheld device may be configured to provide a heating source for applications (e.g., amplification and/or DETECTR reactions) that do not allow for the use of battery or other electrical power supplies (such as in remote locations without easy access to reliable sources of electricity. In certain instances, the handheld device may comprise sodium acetate to provide heating to one or more heating regions of the device, via heat released by the exothermic chemical reaction of sodium acetate. In some embodiments, the design of the handheld device may allow for uniform heating of the amplification and/or DETECTR reaction components by uniformly surrounding the reaction chamber(s) with a sodium acetate heating agent reservoir.

FIG. 185 shows a point of care device for performing a multiplexed programmable nuclease (e.g., DETECTR) assay. FIG. 186 shows an exploded view of point of care device with fluidic chip assembly disposed between chemical heating packs.

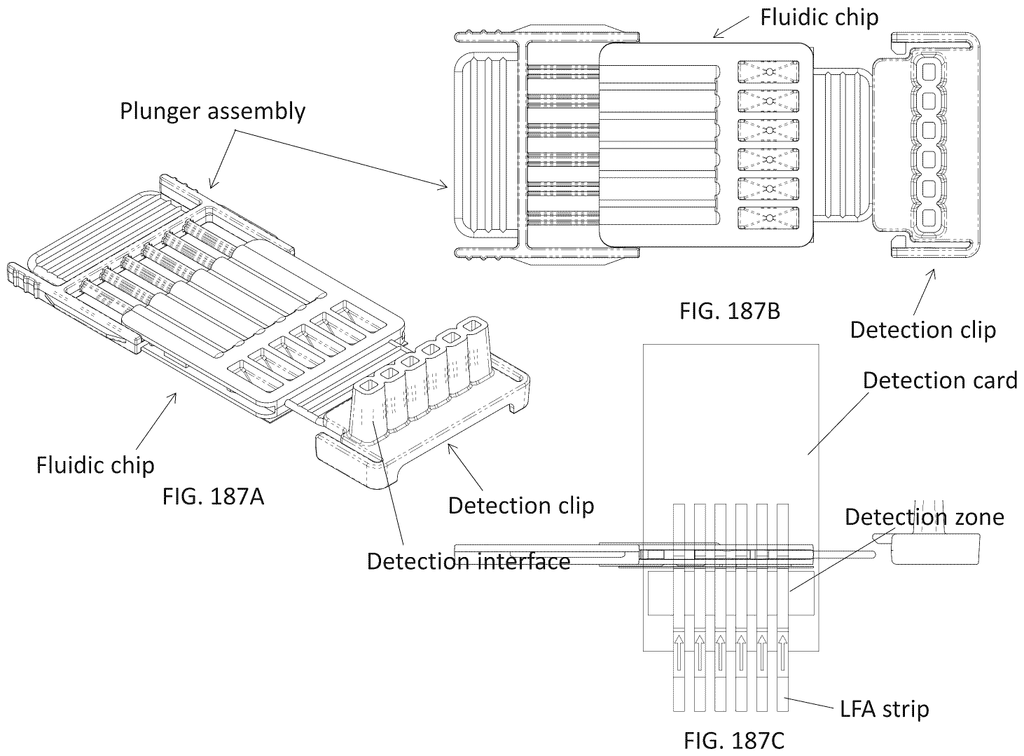

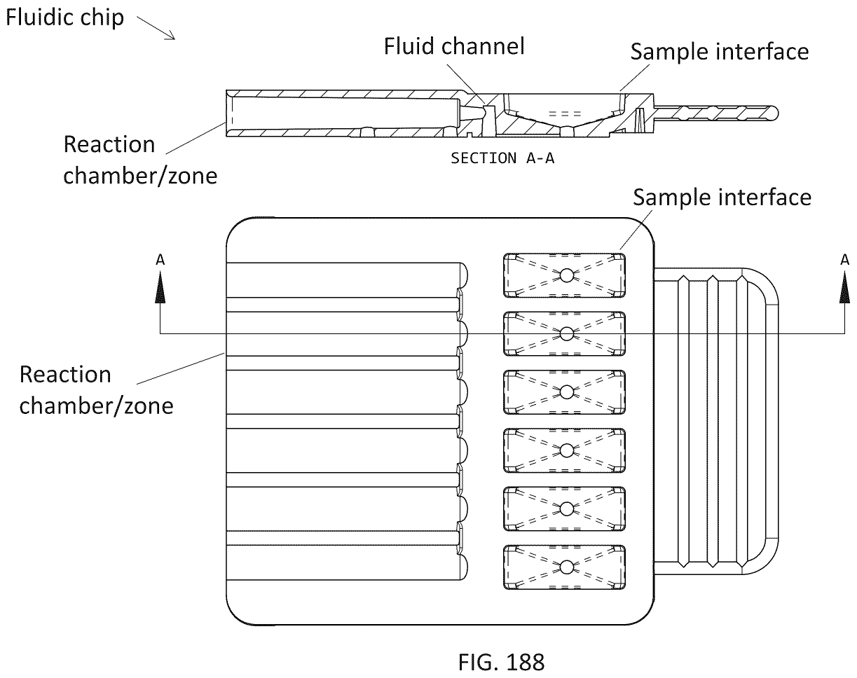

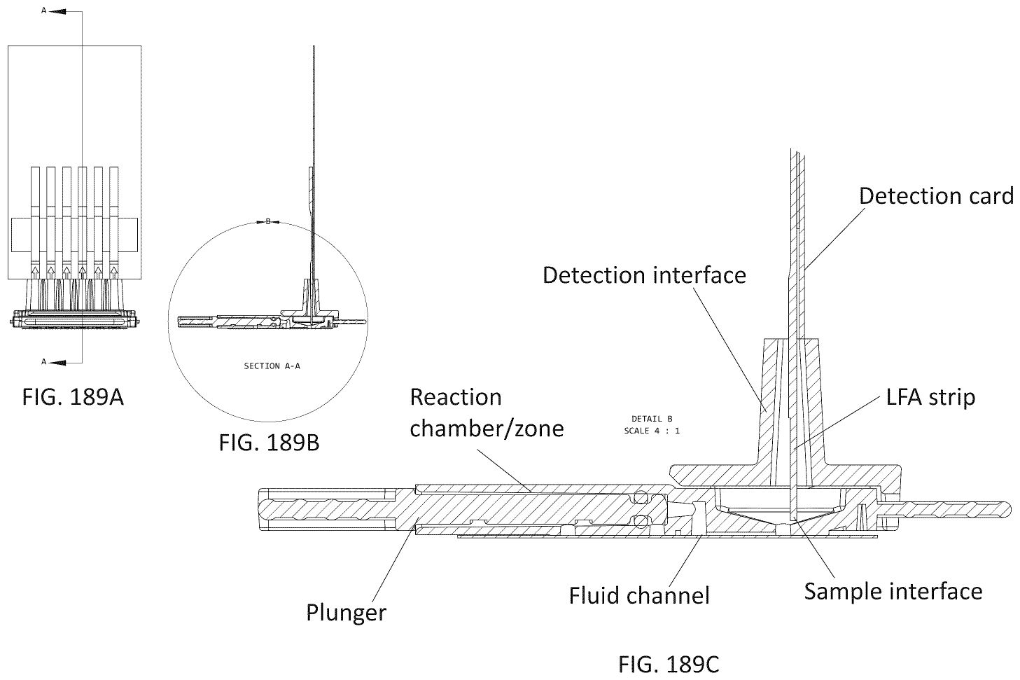

FIGS. 187A-187C show various views of the chip assembly of the device. FIG. 187A shows a perspective view of a chip assembly comprising a fluidic chip, a plunger assembly, and a detection clip. FIG. 187B shows a top view of the chip assembly. FIG. 187C shows a front view of a detection card configured to be inserted into a detection interface of the detection clip. FIG. 188 shows side and top views of the fluidic chip of FIG. 187A. FIGS. 189A-189C show front (FIG. 189A), side (FIG. 189B), and cross-sectional (FIG. 189C) vies of the fully assembled chip assembly of FIG. 185 including a fluidic chip, a plunger assembly, a detection clip, and a detection card.

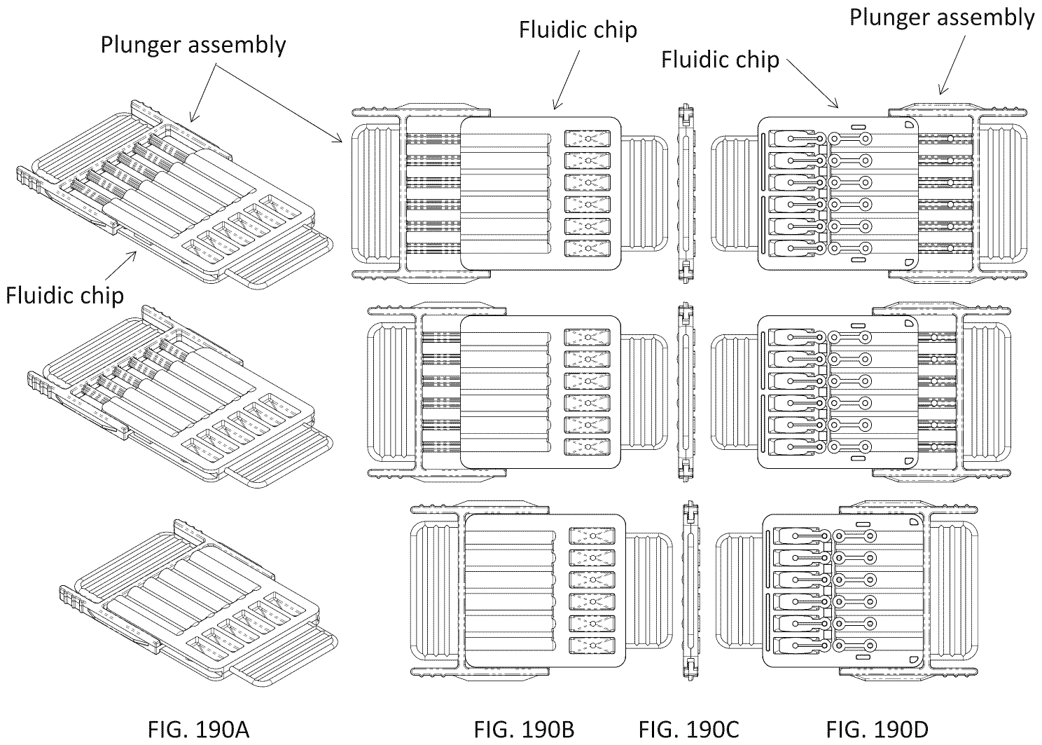

FIGS. 190A-190D show perspective (FIG. 190A), top (FIG. 190B), side (FIG. 190C), and bottom (FIG. 190D) views showing various configurations of the fluidic chip and plunger assembly in use. Prior to use, the plunger assembly may be disposed within the fluidic chip in an initial configuration as shown in the middle row. After the sample is added to the sample interface, the plunger assembly may be pulled back into a loaded configuration to draw the sample into the reaction chamber/channel as shown in the top row. After the amplification and/or DETECTR reactions are run, the plunger assembly may be pushed into a dispensing configuration to push the sample out of the reaction chamber/channel and into the sample interface/detection interface as shown in the bottom row. The detection card comprising lateral flow assay strips may then be inserted into the detection interface and the detection region may be visualized to determine if the target nucleic acid was present in the sample as described herein.

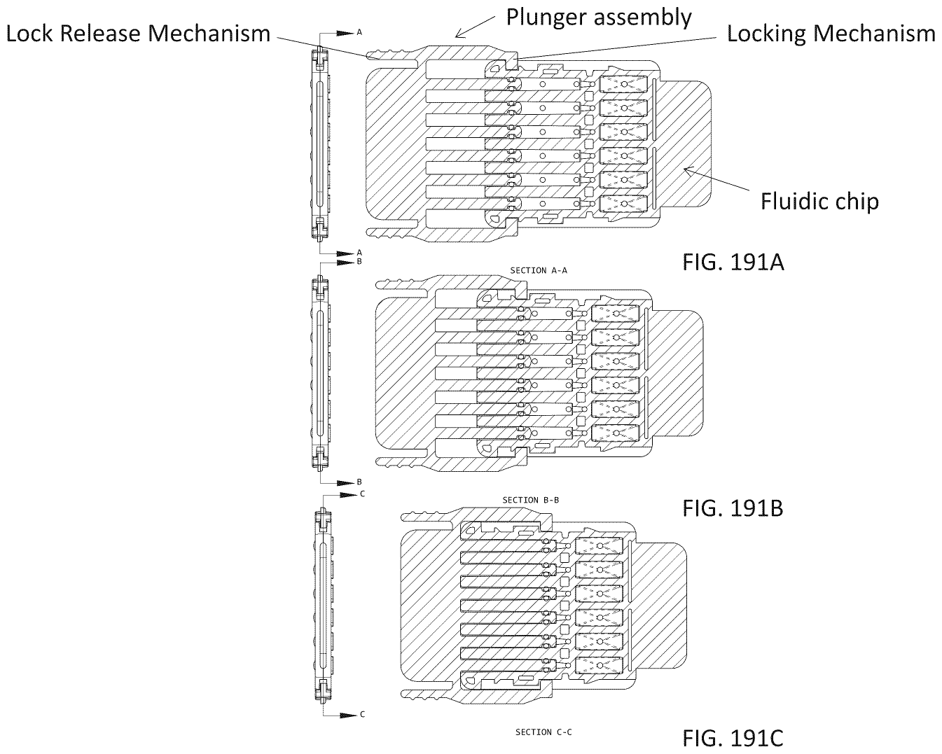

FIGS. 191A-191C show cross-sectional side (left) and top (right) views highlighting various configurations of the fluidic chip and plunger assembly in use. Prior to use, the plunger assembly may be disposed within the reaction chamber/channel of the fluidic chip in an initial configuration as shown in FIG. 191B. After the sample is added to the sample interface, the plunger assembly may be pulled back into a loaded configuration to draw the sample into the reaction chamber/channel as shown in FIG. 191A. A locking mechanism may prevent the plunger assembly from moving during the reaction. After the amplification and/or DETECTR reactions are run, the plunger assembly may be pushed into a dispensing configuration to push the sample out of the reaction chamber/channel and into the sample interface/detection interface as shown in FIG. 191C. A locking release mechanism may be actuated (e.g., by squeezing) to release the locking mechanism and allow the plunger assembly to move between configurations. The detection card comprising lateral flow assay strips may then be inserted into the detection interface and the detection region may be visualized to determine if the target nucleic acid was present in the sample as described herein.

### Enzyme-Linked Signal Amplification Strategies

Disclosed herein are systems, compositions, and methods for amplifying a signal from a target nucleic acid using an enzyme. In some embodiments, enzyme-based assays may allow for signal amplification of a binding event between one or more programmable nuclease probes and one or more target nucleic acids.

Disclosed herein are some strategies that use enzymes with programmable nucleases (e.g., DETECTR based assays) to amplify detection signals indicative of the presence of one or more target nucleic acids in a sample.

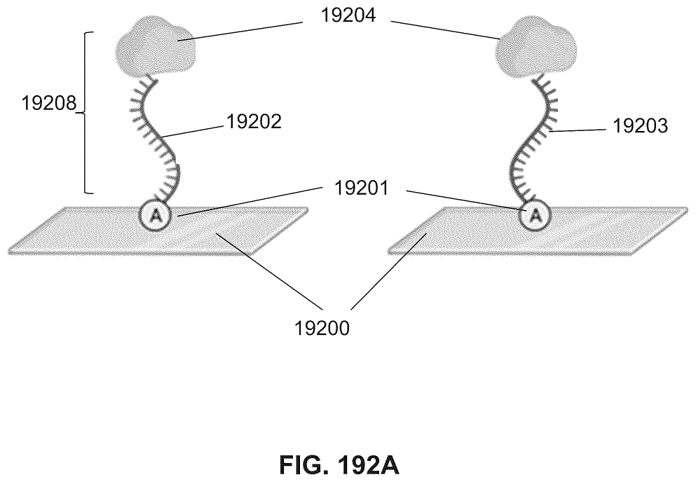

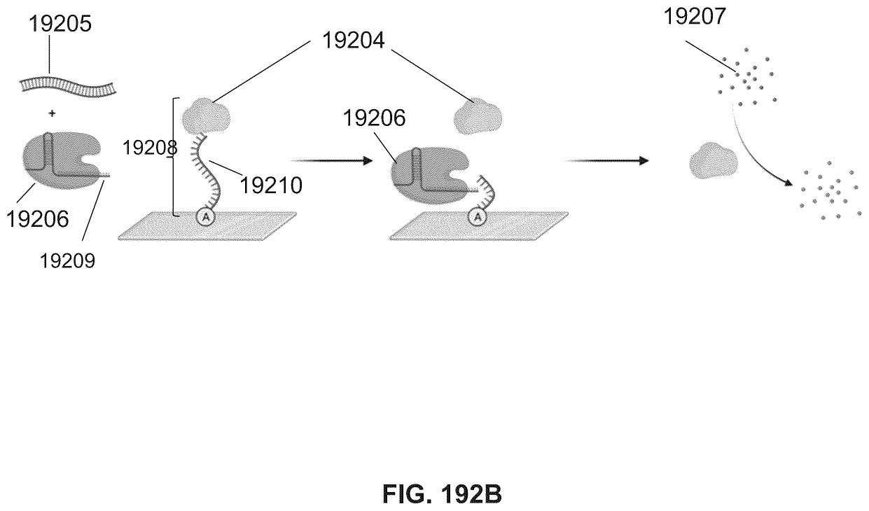

FIGS. 192A-192B show an enzyme-based signal generation and amplification strategy that involves a reporter (19208), wherein the reporter comprises a nucleic acid tether and an enzyme (19204). Activation of a programmable nuclease (19206) upon binding with its guide nucleic acid (19209) and a target nucleic acid (19205), may enable the programmable nuclease to carry out trans-cleavage of the nucleic acid tether of the reporter. The trans-cleavage of the nucleic acid tether may release the enzyme into solution. Release of the enzyme may create a detectable signal, for example, by the enzyme reacting with an enzyme substrate (19207) in a solution to induce a color change in the solution, thereby indicating the presence of the target nucleic acid in the sample.

FIG. 192A shows two exemplary embodiments of a reporter (19208) comprising an enzyme (19204) and a nucleic acid tether, wherein the nucleic acid tether immobilizes the reporter to a surface (19200). In some embodiments, the reporter may comprise an enzyme and a nucleic acid tether, e.g., a ssDNA tether (19202) or a RNA tether (19203). In some embodiments, the nucleic acid tether may comprise a mixture of DNA and RNA.

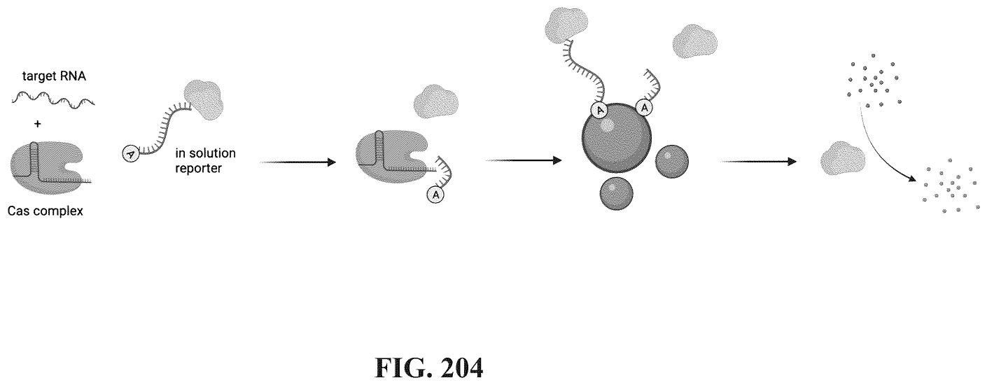

FIG. 192B shows an exemplary signal generation strategy utilizing a reporter (19208) comprising an enzyme (19204) and a nucleic acid tether (19210), wherein the nucleic acid tether immobilizes the reporter to a surface (19200). The first panel from the left shows a programmable nuclease complex, comprising a programmable nuclease (19206) with a guide nucleic acid (19209), and a target nucleic acid (19205) prior to encountering the reporter immobilized to the surface. Upon binding of a target nucleic acid to the programmable nuclease complex, the programmable nuclease may be activated and may cleave the nucleic acid tether of the reporter, thereby releasing the enzyme into solution. In some embodiments, an enzyme substrate (19207) may be added to the solution after the enzyme is cleaved off the reporter, as shown in the last panel from the left. In some embodiments, the solution (or supernatant) containing the released enzyme can be separated and added to a solution comprising an enzyme substrate (19207) upon which the enzyme (19204) can act to produce a detectable signal in the solution. FIG. 204 illustrates an alternative method in which the reporters comprise the linker and functionality for immobilization, but are free in solution at the time of exposure to the programmable nuclease. In the presence of a target nucleic acid, an activated programmable nuclease complex cleaves the reporter, separating the enzyme from the functionality. The reaction is then contacted with beads reactive with the functionality, leaving only released enzyme (if present) in the solution. The solution is then separated from the beads, and exposed to a substrate of the enzyme. If enzyme is present (resulting from cleavage of the reporter in response to presence of the target nucleic acid), that enzyme reacts with its substrate to produce a detectable signal. Without wishing to be bound by theory, reporters that are unbound in solution may be more accessible to activated programmable nuclease, while inclusion of the functionality for immobilization still provides the benefits of surface-bound reagents in later steps. Where reporters are surface-immobilized at the beginning of the reaction, longer linkers may be used to increase accessibility by the activated programmable nuclease.

In some embodiments, the method may comprise (a) providing a solution comprising a target nucleic acid, a programmable nuclease complex configured to bind to the target nucleic acid, and a reporter comprising an enzyme immobilized on a surface, and (b) adding an enzyme substrate configured to be acted on by the enzyme to induce color change of the solution.

In some embodiments, the method may comprise (a) providing a solution comprising a programmable nuclease complex configured to bind to a target nucleic acid and a reporter comprising an enzyme immobilized on a surface, and (b) adding the target nucleic acid and an enzyme substrate configured to be acted on by the enzyme to induce color change of the solution.

In some embodiments, the method may comprise (a) providing a solution comprising a target nucleic acid and a reporter comprising an enzyme immobilized on a surface, and (b) adding a programmable nuclease complex configured to bind to the target nucleic acid and an enzyme substrate configured to be acted on by the enzyme to induce color change of the solution.

In some embodiments, the method may comprise (a) providing a solution comprising a target nucleic acid and a programmable nuclease complex configured to bind to the target nucleic acid, and (b) adding a reporter comprising an enzyme immobilized on a surface and an enzyme substrate configured to be acted on by the enzyme to induce color change of the solution.

In some embodiments, the method may comprise (a) providing a solution comprising a reporter comprising an enzyme immobilized on a surface, and (b) adding a target nucleic acid, a programmable nuclease complex configured to bind to the target nucleic acid, and an enzyme substrate configured to be acted on by the enzyme to induce color change of the solution.

In some embodiments, the method may comprise (a) providing a solution comprising a target nucleic acid, a programmable nuclease complex configured to bind to the target nucleic acid, and an enzyme substrate configured to be acted on by an enzyme to induce color change of the solution, and (b) adding reporter comprising the enzyme immobilized on one or more surfaces.

In some embodiments, the method may comprise (a) providing a solution comprising programmable nuclease complex configured to bind to a target nucleic acid and an enzyme substrate configured to be acted on by an enzyme to induce color change of the solution, and (b) adding the target nucleic acid and a reporter comprising the enzyme immobilized on one or more surfaces.

In some embodiments, the method may comprise (a) providing a solution comprising a target nucleic acid and an enzyme substrate configured to be acted on by an enzyme to induce color change of the solution, and (b) adding a programmable nuclease complex configured to bind to the target nucleic acid and a reporter comprising the enzyme immobilized on one or more surfaces.

In some embodiments, the method may comprise (a) providing a solution comprising substrates configured to be acted on by an enzyme to induce color change of the solution, and (b) adding a target nucleic acid, a programmable nuclease complex configured to bind to the target nucleic acid, and a reporter comprising the enzyme immobilized on one or more surfaces.

In some embodiments, the reporter may comprise any of the reporters disclosed herein. In some embodiments, the programmable nuclease may comprise any one of the programmable nucleases disclosed herein. In some embodiments, the nucleic acid tether may comprise any of the nucleic acid tethers disclosed herein.

The nucleic acid tether of the reporter may be attached to, bound to, coupled to, or otherwise immobilized on the surface using any of the immobilization chemistries or linkages described herein. In some embodiments, the immobilization chemistry or linkage may be between: streptavidin and biotin; an amine group and N-hydroxysuccinimide (NHS), an amine group and an epoxy group, or a malemide group and a thiol group.

In some embodiments, the surface may be any surface disclosed herein. In some embodiments the surface may be of a support as described herein. In some embodiments, the support may comprise glass, PDMS, a hydrogel, plastic, magnetic beads, agarose beads, nitrocellulose, or any other support whose surface can be modified to bind nucleic acids. In some embodiments, the surface of the support, or of a reaction chamber, comprises streptavidin, biotin, an amine group, a carboxyl group, an epoxy group, an NHS group, a malemide group, or a thiol group. In some embodiments, the target nucleic acid may be any target nucleic acid disclosed herein. In some embodiments, the target nucleic acid may be a single-stranded DNA, a single-stranded RNA, a double-stranded DNA, a double-stranded RNA, or a double-stranded DNA/RNA hybrid.

In some embodiments, the guide nucleic acid may be any guide nucleic acid disclosed herein. In some embodiments, the guide nucleic acid may be a single-stranded DNA or a single-stranded RNA. In some embodiments, the nucleic acid tethering the reporter may comprise any of the reporter nucleic acids described herein.

In some embodiments, the enzyme may be HRP, AP, beta-galactosidase, acetylcholinesterase, catalase, catacolase, tyronase, nitrocefelin, alkaline phosphatase, or invertase. In some embodiments, the enzyme may be any one of the enzymes described herein.

In some embodiments, the enzyme may bind with an enzyme substrate and produce a detectable signal. In some embodiments, the enzyme substrate may be 3,3′,5,5′-tetramethylbenzidine (TMB), 2,2′-Azinobis [3-ethylbenzothiazoline-6-sulfonic acid]-diammonium salt (ABTS), o-phenylenediamine dihydrochloride (OPD), p-Nitrophenyl Phosphate (PNPP), o-nitrophenyl-β-D-galactopyranoside (ONPG), 3,3′-diaminobenzidine (DAB), p-hydroxyphenylacetic acid, 3-(p-hydroxyphenyl)-propionic acid, homovanillic acid, or o-aminophenol. In some embodiments, the enzyme substrate may be a commercial enzyme substrate including SuperSignal ELISA Pico, SuperSignal Elisa Femto, CDP-Star Substrate, CSPD Substrate, DynaLight Substrate with RapidGlow Enhancer, QuantaBlu, QuantaRed, or Amplex.

In some embodiments, the enzyme may generate a colorimetric signal, a fluorescent signal, an electrochemical signal, a chemiluminescent signal, or another type of signal. In some embodiments, the enzyme may induce color-change in substances.

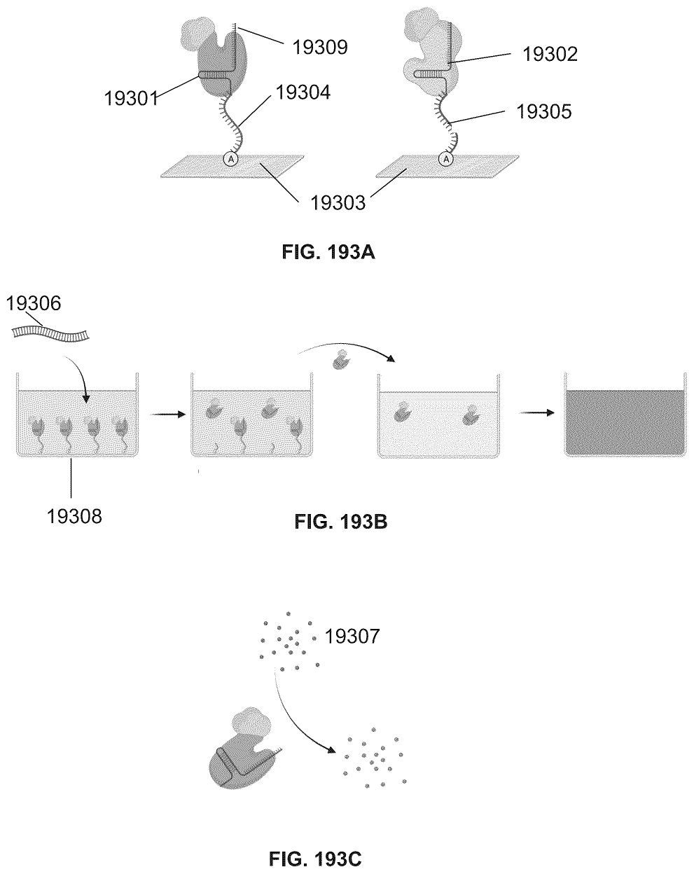

FIGS. 193A-193C show a signal generation and amplification strategy involving the release of a programmable nuclease-enzyme fusion protein comprising a programmable nuclease and an enzyme. Presence of a target nucleic acid may activate the programmable nuclease of the programmable nuclease-enzyme fusion protein, wherein the activated programmable nuclease may cleave a nucleic acid tether that immobilizes the programmable nuclease-enzyme fusion protein on a surface. Cleaving the nucleic acid tether may release the programmable nuclease-enzyme fusion protein into solution, wherein the enzyme of the programmable nuclease-enzyme fusion protein may be contacted with an enzyme substrate to create a detectable and amplified signal.

FIG. 193A shows two exemplary embodiments of a programmable nuclease-enzyme fusion protein comprising (i) an enzyme portion and (ii) a programmable nuclease portion complexed with a guide nucleic acid. In some embodiments, the programmable nuclease-enzyme fusion protein may be complexed with a guide nucleic acid (19309) tethered or immobilized to a surface (19303) as described herein. In some embodiments, the guide nucleic acid may be immobilized to the surface with a ssDNA tether (19304). In some embodiments, the guide nucleic acid may be immobilized to the surface with an RNA tether (19305).

In some embodiments, the programmable nuclease of the programmable nuclease-enzyme fusion protein may be Cas12, Cas13, Cas14, or CasPhi. In some embodiments, the programmable nuclease of the programmable nuclease-enzyme fusion protein may be any programmable nuclease disclosed herein.

In some embodiments, the enzyme of the programmable nuclease-enzyme fusion protein may be HRP, AP, beta-galactosidase, acetylcholinesterase, or catalase. In some embodiments, the enzyme of the programmable nuclease-enzyme fusion protein may be any enzyme disclosed herein.

In some embodiments, the programmable nuclease may be fused by a peptide linker to the enzyme. In some embodiments, the C-terminus of the programmable nuclease portion may be fused by a peptide linker to the N-terminus of the enzyme. In some embodiments, the N-terminus of the programmable nuclease portion may be fused by a peptide linker to the C-terminus of the enzyme. In some embodiments, the peptide linker may be XTEN.

FIG. 193B shows an example procedure using a programmable nuclease-enzyme fusion protein comprising (i) an enzyme and (ii) a programmable nuclease complexed with a guide nucleic acid. In some embodiments, a target nucleic acid (19306) may be added to a solution comprising the programmable nuclease-enzyme fusion protein bound to an immobilized guide nucleic acid. The target nucleic acid may bind with the programmable nuclease-enzyme fusion protein on the surface of a detection location (19308) and activate the trans-cleavage activity of the programmable nuclease of the programmable nuclease-enzyme fusion protein. The activated programmable nuclease may then cleave the nucleic acid tether (e.g., a ssDNA tether or a RNA tether) and release the programmable nuclease-enzyme fusion protein into solution (in a manner analogous to release of a detection moiety from a reporter as described herein). The solution (or the supernatant) comprising the released programmable nuclease-enzyme fusion protein can be added to a second solution comprising an enzyme substrate (19307) which can be acted upon by the enzyme of the programmable nuclease-enzyme fusion protein to produce a detectable signal as described herein (e.g., to induce a color change of the second solution) as shown in FIG. 193C.

In some embodiments, the method may comprise (a) providing a solution comprising an enzyme substrate and a programmable nuclease-enzyme fusion protein immobilized on a surface, wherein the programmable nuclease-enzyme fusion protein comprises a programmable nuclease and an enzyme, wherein the programmable nuclease is configured to bind to a target nucleic acid, and (b) adding the target nucleic acid.

In some embodiments, the method may comprise (a) providing a solution comprising an enzyme substrate and a target nucleic acid, and (b) adding a programmable nuclease-enzyme fusion protein immobilized on a surface, wherein the programmable nuclease-enzyme fusion protein comprises a programmable nuclease and an enzyme, wherein the programmable nuclease is configured to bind to a target nucleic acid.

In some embodiments, the programmable nuclease-enzyme fusion protein may comprise any one of the programmable nucleases disclosed herein. In some embodiments, the target nucleic acid may be any one of the target nucleic acids disclosed herein. In some embodiments, the guide nucleic acid may comprise any one of the guide nucleic acids disclosed herein. In some embodiments, the nucleic acid tethering the guide nucleic acid to the surface may comprise any of the reporter nucleic acids described herein. In some embodiments, the programmable nuclease-enzyme fusion protein may comprise any of the enzymes described herein.

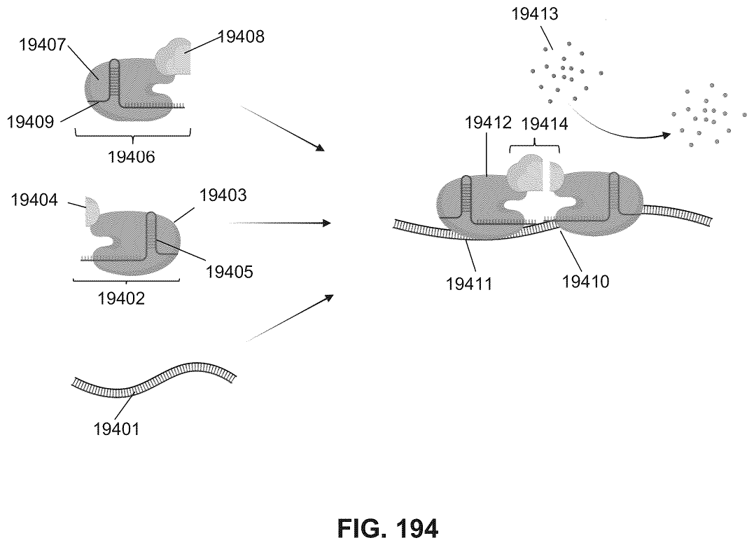

FIG. 194 shows an exemplary signal generation strategy utilizing a split-enzyme based assay strategy. A first programmable nuclease-split enzyme fusion protein (19402) may comprise a first catalytically inactive programmable nuclease (19403), e.g., a first dead Cas (dCas) protein, fused to a first subunit (19404) of a split enzyme. A second programmable nuclease-split enzyme fusion protein (19406) may comprise a second catalytically inactive programmable nuclease (19407), e.g., a second dead Cas (dCas) protein, fused to a second subunit (19408) of the split enzyme. The first catalytically inactive programmable nuclease (19403) may be complexed to a first guide nucleic acid (19405) configured to bind to a first portion or sequence (19410) of a target nucleic acid. The second catalytically inactive programmable nuclease (19407) may be complexed to a second guide nucleic acid (19409) configured to bind to a second portion or sequence (19411) of a target nucleic acid. The first portion (19410) and the second portion (19411) of the target nucleic acid may be sufficiently close to bring the first subunit (19404) and the second subunit (19408) of the split enzyme together to form a functional enzyme (19414). Once activated, the functional enzyme (19414) may act upon an enzyme substrate (19413) to generate a detectable signal as described herein.

In some embodiments, a catalytically inactive programmable nuclease may have no substantial nucleic acid-cleaving activity. In some embodiments, a catalytically inactive programmable nuclease may be catalytically inactive or “dead,” that is, they may bind to a target nucleic acid but not cleave the target nucleic acid. A catalytically inactive or dead programmable nuclease may comprise a catalytically inactive domain (e.g., inactive nuclease domain). In some instances, a catalytically inactive domain may comprise at least 1, at least 2, at least 3, at least 4, or at least 5 mutations relative to an active domain of a catalytically active programmable nuclease. In some instances, the mutations may be present within a cleaving or active site of the programmable nuclease. In some instances, a catalytically inactive or dead programmable nuclease is fused to a fusion partner. In some instances, catalytically inactive may refer to an activity less than 1%, less than 2%, less than 3%, less than 4%, less than 5%, less than 6%, less than 7%, less than 8%, less than 9%, or less than 10% activity compared to a wild-type counterpart of the programmable nuclease.

In an exemplary embodiment, a target nucleic acid (19401) may bind with a programmable nuclease-split enzyme fusion protein comprising a dCas protein, an “S” subunit of a split HRP protein, and a first guide nucleic acid. In some embodiments, the dCas protein is not capable of cis or trans cleavage. The target nucleic acid (19402) may also bind with another programmable nuclease-split enzyme fusion protein (19406) comprising a dCas protein (19407), an “L” subunit of a split HRP protein (19408), and a second guide nucleic acid (19409). The first guide nucleic acid (19402) may be configured to target a first portion (19410) of the target nucleic sequence. The second guide nucleic acid (19409) may be configured to target a second portion (19411) of the target nucleic acid sequence, wherein the second portion is sufficiently close to the first portion such that the “S” subunit of the HRP (19404) and the “L” subunit of the HRP (19408) can dock together to form an activated HRP protein (19412). The activated HRP protein (19412) then may act on an enzyme substrate (19413) in solution to induce a color change.

In some embodiments, the programmable nuclease-split enzyme fusion protein may comprise any one of the programmable nucleases disclosed herein. In some embodiments, the target nucleic acid may be any one of the target nucleic acids disclosed herein. In some embodiments, the guide nucleic acids may comprise any of the guide nucleic acids disclosed herein. In some embodiments, the programmable nuclease-split enzyme fusion protein may comprise any of the enzymes described herein in split form.

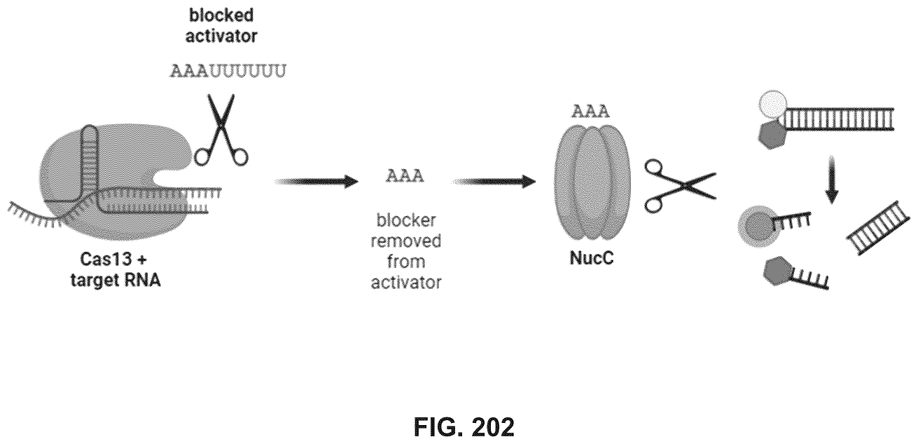

FIG. 202 shows a schematic of a signal amplification strategy involving downstream activation of an endonuclease (e.g., NucC, Csm6, etc.) following activation of a programmable nuclease (e.g., any of the programmable nucleases described herein) in response to a target nucleic acid. The programmable nuclease may be configured to cleave a first reporter comprising a first nucleic acid section and a second nucleic acid section. The first nucleic acid section may act as an activator for the endonuclease when separated from the second nucleic acid section. The second nucleic acid section may act as a blocker nucleic acid and prevent the first nucleic acid from binding to and/or activating the endonuclease. Presence of the target nucleic acid may activate the programmable nuclease, which may cleave the first nucleic acid section from the second nucleic acid section, thereby freeing the first nucleic acid section to bind to and activate the endonuclease. The activated endonuclease may then cleave the second reporter and release a detection moiety where may be detected as described herein.

In one aspect, the present disclosure provides a composition for detecting a target nucleic acid, in which the composition comprises a programmable nuclease, a guide nucleic acid, a first reporter, an enzyme, and a second reporter. In some embodiments, (a) the guide nucleic acid is configured to bind to the target nucleic acid; (b) the programmable nuclease is effective to cleave the first reporter in response to formation of a complex comprising the programmable nuclease, the guide nucleic acid, and the target nucleic acid; (c) the cleavage of the first reporter is effective to separate a first nucleic acid section from a second nucleic acid section thereof; (d) the first nucleic acid section is effective to activate the enzyme; and (e) the activated enzyme is effective to cleave the second reporter to produce a detectable product comprising a detection moiety. In some embodiments, the programmable nuclease is a Cas protein or fragment thereof. In some embodiments, the programmable nuclease is a Cas13. In some embodiments, the programmable nuclease is a Cas13a. In some embodiments, the programmable nuclease is a Cas13c. In some embodiments, the programmable nuclease is thermostable at elevated temperatures. In some embodiments, the enzyme is an endonuclease and the second reporter comprises a polynucleotide substrate of the enzyme. In some embodiments, the endonuclease is a NucC endonuclease. In some embodiments, the first nucleic acid section comprises adenosine residues (such as cyclic adenylate) effective to activate the NucC. In some embodiments, the second nucleic acid section comprises RNA residues, optionally wherein the RNA residues comprise a plurality of uracil residues (e.g., at least 4, 5, 6, 7, 8, 9 or 10 RNA residues, such as uracils). In some embodiments, the second nucleic acid section comprises DNA residues, optionally wherein the DNA residues comprise a plurality of thymine residues (e.g., at least 4, 5, 6, 7, 8, 9 or 10 DNA residues, such as thymines). In some embodiments, (a) the second reporter comprises a fluorescent label and a quencher, and (b) cleavage of the second reporter by the activated enzyme is effective to separate the fluorescent label from the quencher.

In some embodiments, such as when the endonuclease is NucC, the first reporter may comprise a first nucleic acid section comprising a plurality of adenosine residues and a second nucleic acid section comprising a plurality of uracil residues. In some embodiments, one or more of the plurality of adenosine residues may be modified (e.g., fluoro-adenosine, etc.) as described herein. When the target nucleic acid is a target RNA, the programmable nuclease may comprise a Cas13 nuclease which, when activated by the target RNA, may preferentially cleave the uracil residues off of the first reporter and release the first nucleic acid section comprising the adenosine residues. NucC is naturally activated by cyclic adenylate (cA3) and cleaves double-stranded DNA, thus in an exemplary embodiment the plurality of adenosine residues may be at least two or three adenosine residues and the second reporter may comprise a detection moiety (e.g., a fluorophore) and a nucleic acid comprising double-stranded DNA. In some embodiments, the second reporter may comprise a detection moiety (e.g., a fluorophore) and a quencher moiety. Cleavage of the second reporter may separate the detection moiety from the quencher moiety and generate a detectable signal as described herein.

In some embodiments, such as when the endonuclease is NucC, the composition for detecting the target nucleic acid and the target nucleic acid are reacted and analyzed at room temperature. In some embodiments, the composition for detecting the target nucleic acid and the target nucleic acid are reacted and analyzed at an elevated temperature. In some embodiments, the elevated temperature is a temperature selected from: 40° C., 45° C., 50° C., 55° C., 60° C., or 65° C.

In various aspects, provided herein are methods for detecting a target nucleic acid in a sample, the methods comprising contacting the sample with a composition, device, or system described herein. In an embodiment, the method comprises (a) contacting the sample with composition described herein; (b) cleaving the first reporter with the programmable nuclease in response to formation of the complex comprising the programmable nuclease, the guide nucleic acid, and the target nucleic acid, thereby releasing the first nucleic acid section; (c) activating the enzyme with the first nucleic acid section; (d) cleaving the second reporter with the activated enzyme, thereby producing the detectable product comprising the detection moiety; and; and (e) detecting the detection moiety, thereby detecting the presence of the target nucleic acid in the sample.

### Enzyme-Based Reporters for DETECTR Signal Enhancement

Described herein are various compositions, devices, and methods for detecting a target nucleic acid, utilizing a DETECTR assay as described herein. In some embodiments, the DETECTR assay utilizes reporters comprising one or more detection moieties. One or more of the detection moieties may comprise an enzyme configured for assay signal enhancement as described herein. In some embodiments, the enzyme is horseradish peroxidase (HRP). In some embodiments, enzyme-modified reporters may be immobilized to a surface and configured to release the enzyme upon cleavage of a nucleic acid of the reporter by an activated programmable nuclease-guide complex bound to a target nucleic acid as described herein. In some embodiments, one or more enzymes are conjugated to a linker, the comprises one or more nucleic acid sections, and the linker comprises a functionality for immobilization to a support (but is not immobilized to the support at the time of cleavage by an activated programmable nuclease). In some embodiments, the reporters may comprise two or more enzymes configured for signal enhancement. In some embodiments, a reporter comprising two or more enzymes configured for signal enhancement is referred to as a poly-enzyme reporter. In some embodiments, each of the two or more enzymes configured for signal enhancement are HRP. In some embodiments, wherein the two or more enzymes configured for signal enhancement are each HRP, the reporter is referred to as a poly-HRP reporter.

Described herein are various devices for detecting a target nucleic acid, comprising: a sample interface configured to receive a sample comprising the target nucleic acid; a first reaction chamber in fluid communication with the sample interface, wherein the first reaction chamber comprises a plurality of reporters, each reporter of the plurality comprising two or more enzymes; and a surface of the first reaction chamber, wherein each reporter of the plurality of reporters is immobilized to the surface of the first reaction chamber via a nucleic acid (which may comprise or be a part of a linker or tether as described herein), and a second reaction chamber in fluid communication with the first reaction chamber, wherein the second reaction chamber comprises an enzyme substrate, and wherein cleaving the nucleic acid of the reporter by a programmable nuclease complexed with a guide nucleic acid, the guide nucleic acid being configured to bind to the target nucleic acid, may release the two or more enzymes into solution, and wherein each enzyme of the two or more enzymes may be contacted with the enzyme substrate in the second reaction chamber to generate a detectable and amplified signal indicative of a presence or absence of the target nucleic acid in the sample. In some embodiments, each enzyme of the two or more enzymes comprises HRP. In some embodiments, the reporter comprises a detection moiety comprising a streptavidin conjugated to the two or more enzymes. In some embodiments, the nucleic acid of the reporter may be immobilized to the surface via a functionality (e.g., a biotin, an acrydite, an amine, or the like) as described herein. In some embodiments, the surface of the first reaction chamber may be functionalized with a corresponding functionality (e.g., a hydrogel, carboxyl group, NHS groups, or like) as described herein. In some embodiments, the tether comprises a segment(s) of cleavable DNA, cleavable RNA, or a combination thereof. In some embodiments, the tether comprises a segment(s) of non-cleavable DNA, non-cleavable RNA, or a combination thereof. In some embodiments, the enzyme comprises HRP and the enzyme substrate comprises 3,3′,5,5′-tetramethylbenzidine (TMB). In some embodiments, the surface of the first reaction chamber comprises the inner wall of the first reaction chamber. In some embodiments, the surface of the first reaction chamber comprises a surface of a bead, wherein the bead is contained within the first reaction chamber. In some embodiments, the bead is a magnetic bead.

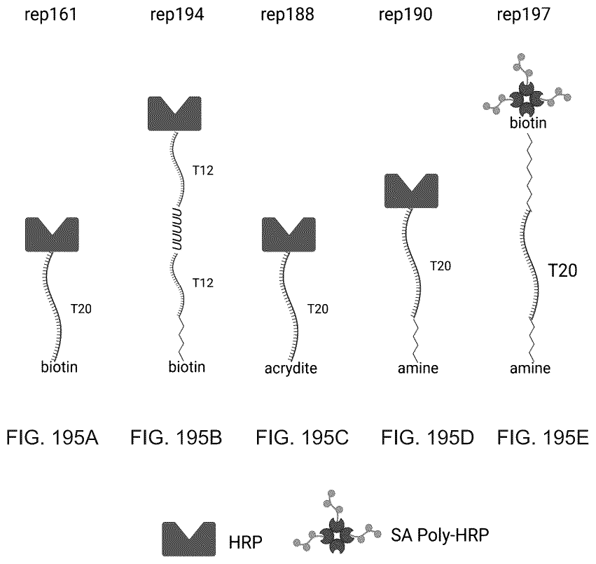

Described herein are various devices and methods for detecting a target nucleic acid, utilizing a DETECTR assay as described herein. In some embodiments, the DETECTR assay utilizes reporters comprising a detection moiety comprising an enzyme configured for assay signal enhancement as described herein. Non-limiting examples of enzymes for assay signal enhancement include horseradish peroxidase (HRP), alkaline phosphatase, beta-galactosidase, catacholase, invertase, glucose, beta-lactamase, luciferase, and complexes comprising two or more thereof (e.g., two or more of the same enzyme, or two or more different enzymes). In some embodiments, the enzyme is HRP. In some embodiments, the enzyme is a poly-HRP. In some embodiments, enzyme-modified reporters may be immobilized to a surface and configured to release the enzyme upon cleavage of a nucleic acid of the reporter by an activated programmable nuclease-guide complex bound to a target nucleic acid as described herein. In some embodiments, the reporters comprise two or more enzymes configured for signal enhancement. In some embodiments, the incorporation of the two or more enzymes configured for signal enhancement may provide for assay signal enhancement upon a single cleavage event of the reporter induced by the activated programmable nuclease. In some embodiments, the two or more enzymes comprise a group, wherein the group further comprises a protein. In some embodiments, the two or more enzymes comprise a group, wherein the group further comprises a protein, the protein comprising streptavidin (SA). In some embodiments, the group comprises two or more HRP. In some embodiments, the group comprising two or more enzymes is attached to the nucleic acid of the reporter via a Biotin-Streptavidin interaction (e.g., as shown in FIG. 195E).

In some embodiments, the reporter comprises a linker (also referred to herein as a tether). In some embodiments, the linker comprises one or more linker-sections. In some embodiments, the one or more linker sections comprises a cleavable section comprising one or more nucleic acids which may be cleaved by an activated programmable nuclease as described herein. In some embodiments, the one or more linker sections comprises cleavable and non-cleavable section, the non-cleavable section comprising one or more nucleic acids, carbon spacers, or other molecules which may not be cleaved by an activated programmable nuclease. For example, FIG. 195B shows an exemplary reporter comprising a linker comprising a carbon spacer, a first linker section comprising a first plurality of thymines (T12 (SEQ ID NO: 127)), a second linker section comprising a plurality of uracils (UUUUU), and a third linker section comprising a second plurality of thymines (T12 (SEQ ID NO: 127)). The carbon spacer may be uncleavable by the programmable nuclease. When the programmable nuclease is a DNA-targeting programmable nuclease, the first and second pluralities of thymines may be cleavable linker sections and the plurality of uracils may be a non-cleavable linker section. When the programmable nuclease is an RNA-targeting programmable nuclease, the first and second pluralities of thymines may be non-cleavable linker sections and the plurality of uracils may be a cleavable linker section.

In some embodiments, the linker comprises one or more nucleic acid sections, and the only nucleic acids in the linker consist of DNA. In some embodiments, the linker comprises one or more nucleic acid sections, and the only nucleic acids in the linker consist of RNA. In some embodiments, the linker comprises one or more sections of DNA and one or more sections of RNA (e.g., one section of each, one section of RNA between two sections of DNA, one section of DNA between two sections of RNA, or greater numbers of each). In linkers comprising DNA and RNA, the DNA and RNA may be linked (e.g., covalently linked) directly to one another, or separated by one or more intervening molecules or linkages to which both the DNA and RNA are linked (e.g., covalently linked). The type, length, and arrangement of the one or more nucleic acid sections can be selected based on the programmable nuclease(s) to be used. In some embodiments, one or more DNA sections comprise one or more poly-T sections consisting of 2, 3, 4, 5, 6, 7, 8, 9, 10, 11, 12, 13, 14, 15, 20, 25, 30, or more contiguous thymine nucleotides. In some embodiments, the poly-T section consists of 12 contiguous thymine nucleotides (T12) (SEQ ID NO: 127). In some embodiments, the poly-T section consists of 20 contiguous thymine nucleotides (T20) (SEQ ID NO: 126). In a linker comprising two or more poly-T sections, the poly-T sections may be of the same or different lengths (e.g., two of T12 (“T12” disclosed as SEQ ID NO: 127), a T12 (SEQ ID NO: 127) and a T20 (SEQ ID NO: 126), etc.). In some embodiments, one or more RNA sections comprise one or more poly-U sections consisting of 2, 3, 4, 5, 6, 7, 8, 9, 10, 11, 12, 13, 14, 15, 20, 25, 30, or more contiguous uracil nucleotides. In some embodiments, the poly-U section consists of 5 contiguous uracil nucleotides (UUUUU). In a linker comprising two or more poly-U sections, the poly-U sections may be of the same or different lengths (e.g., two of UUUUU, a UUUUU and a U8, etc.) In some embodiments, the RNA section is about or less than about 20, 15, 10, or 5 nucleotides in length. In some embodiments, a linker includes only one RNA section of fewer than 20 nucleotides (e.g., about 5 nucleotides) and is adjusted to a desired length by the inclusion of one or more DNA sections (e.g., at one end or at each end). In some embodiments, the desired length adjustment is the length of 24 DNA nucleotides (e.g., as in two T12 (“T12” disclosed as SEQ ID NO: 127) sections), 40 nucleotides (e.g., as in two T20 (“T20” disclosed as SEQ ID NO: 126) sections), a length intermediate to these (e.g., a T12 (SEQ ID NO: 127) and a T20 (SEQ ID NO: 126)), or another length as specified herein. In some embodiments, the length is adjusted to a combined length of at least about 20, 30, 40, 50, 75, 100, or 200 nucleotides. In some embodiments, the length is adjusted to a combined length of about 10 to 200 nucleotides, 20 to 100 nucleotides, or 25 to 75 nucleotides. In some embodiments, the length is adjusted to a combined length of at least about 20, 30, 40, or 50 nucleotides. In some embodiments, the length is adjusted to a combined length of at least about 30 nucleotides. In some embodiments, the one or more nucleic acid sections are single-stranded.

In some embodiments, the one or more linker sections comprise at least one hydrocarbon chain. In some embodiments, the one or more hydrocarbon chains comprise a linear chain of 2, 3, 4, 5, 6, 7, 8, 9, 10, 20, 50, 75 or more carbons. In some embodiments, a hydrocarbon chain comprises one or more units (e.g., 2, 3, 4, 5, or more units) of an 18-carbon chain. In some embodiments the hydrocarbon chain is a chain of 4 carbons (C₄H₈). In some embodiments the hydrocarbon chain is a chain of 8 carbons (C₈H₁₆). In some embodiments, the hydrocarbon chain is a chain of at least 36 carbons. In some embodiments, the hydrocarbon chain is a chain of at least 54-carbon chain. In a linker comprising two or more hydrocarbon chains, the hydrocarbon chains can be chains of the same or different lengths (e.g., two chains of 4 carbons, a chain of 4 carbons and a chain of 8 carbons, a chain of 36 carbons and a chain of 54 carbons, etc.). In some embodiments of a linker comprising two hydrocarbon chains separated by one or more nucleic acid sections, the two hydrocarbon chains have a combine length of about or at least about 30, 50, 75, 100, or more carbons. In some embodiments, the combined chain length is at least 75 carbons. In some embodiments, a hydrocarbon chain is located at one or more of (i) between a nucleic acid section of a linker and a solid support to which the linker is attached, (ii) between a nucleic acid section of a linker and an enzyme for signal enhancement (e.g., an HRP or poly-HRP), or (iii) between two nucleic acid sections of the same or different type. In some embodiments, the linker comprises a single nucleic acid section (e.g., an RNA section) with a hydrocarbon chain at both ends, one joining the nucleic acid section to the support at one end, and the other joining the nucleic acid section to the enzyme at the other end. In some embodiments, a linker includes only one RNA section of fewer than 20 nucleotides (e.g., about 5 nucleotides) and is adjusted to a desired length by the inclusion of one or more hydrocarbon chains (e.g., at one end or at each end). In some embodiments one or more hydrocarbon chains and one or more DNA sections are used to adjust the linker length.

In some embodiments, the reporter may comprise a functionality for binding to a solid support as described herein. In some embodiments, the reporter may comprise a functionality for binding to a surface of a solid support as described herein. In some embodiments, the surface may comprise a surface of a chamber, the surface of a particle, a bead, a polymer matrix, or other scaffold as described herein. In some embodiments, the functionality may comprise a biotin, an acrydite, an amine, or the like, or a combination thereof, as described herein. FIGS. 195A-195E show various non-limiting examples of reporters, each comprising at least one enzyme for signal enhancement of the DETECTR assay. FIG. 195A shows a reporter, rep161, comprising an HRP enzyme for signal enhancement, a T20 linker (SEQ ID NO: 126) segment, and a biotin for immobilization to a support, as described herein. FIG. 195B shows a reporter, rep194, comprising an HRP enzyme for signal enhancement, a T12 (SEQ ID NO: 127) linker segment, a UUUUU linker segment, a second T12 (SEQ ID NO: 127) linker segment and a biotin functionality for immobilization to a support. FIG. 195C shows a reporter, rep188, comprising an HRP enzyme for signal enhancement, a T20 linker (SEQ ID NO: 126) segment and an acrydite functionality for immobilization to a support. FIG. 195D shows a reporter, rep 190, comprising an HRP enzyme for signal enhancement, a T20 linker (SEQ ID NO: 126) segment, a carbon spacer, and an amine functionality for immobilization to a support. FIG. 195E shows a reporter, rep197, comprising an SA Poly-HRP for signal enhancement, a first carbon spacer, a T20 linker (SEQ ID NO: 126) segment, a second carbon spacer, and an amine functionality for immobilization to a support.

Table 4 describes reporters as shown in FIG. 195.

| Reporter | |
|---|---|
| Name | Description |
| rep161 | HRP-[T20]-biotin (SEQ ID NO: 1498) |
| rep194 | HRP-[T12]-UUUUU-[T12]-[C₄H₈]-biotin (SEQ ID NO: 1499) |
| rep188 | HRP-[T20]-acrydite (SEQ ID NO: 1500) |
| rep190 | HRP-[T20]-[C₄H₈]-amine (SEQ ID NO: 1501) |
| rep197 | Streptavidin-poly-HRP-biotin-[C₈H₁₆]-[T20]-[C₄H₈]-amine |
| | (SEQ ID NO: 1502) |

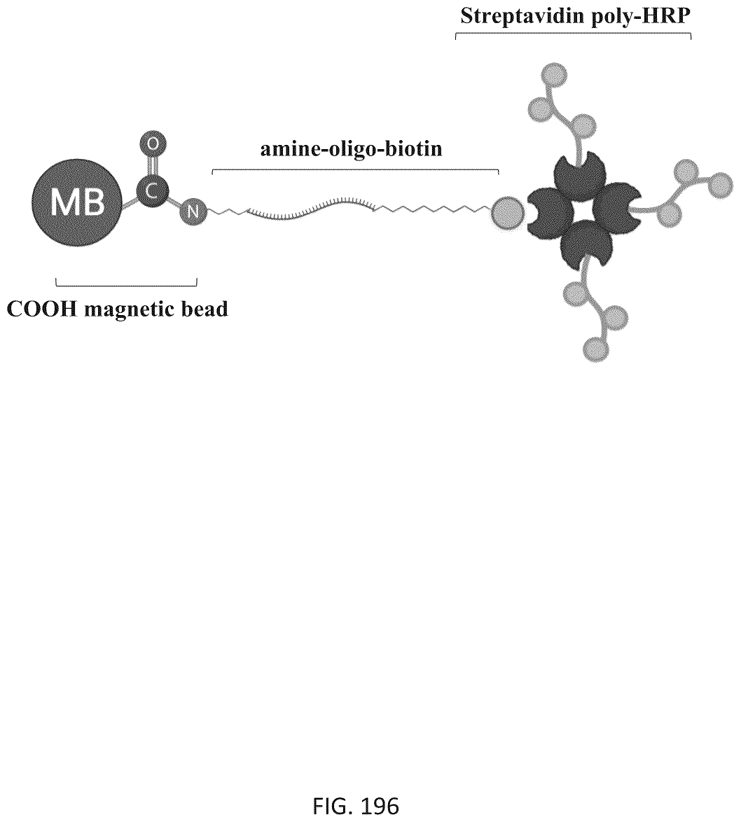

FIG. 196 shows a non-limiting example of a reporter comprising a streptavidin-based poly-HRP detection moiety coupled to a carboxyl-functionalized magnetic bead. The multiple HRP units released per trans-cleavage event is designed to increase signal intensity indicative of activation of the programmable nuclease. In general, the term “poly-HRP” is used to refer to a plurality of HRP enzymes complexed to one another by interactions with one or more complexing molecules. Typically, the complexing molecule is streptavidin. Illustrations of poly-HRP, such as in the figures, are simplified for illustrative purposes. The term “poly-HRP” does not require any particular stoichiometry or arrangement of molecules within the complex. A variety of suitable poly-HRP complexes are commercially available.

In some embodiments, the reporter may be immobilized onto a solid support as described herein. In some embodiments, the reporter may be a poly-enzyme reporter. In some embodiments, one or more of the poly-enzymes of the reporter may be HRP. In some embodiments, the poly-HRP reporter may be immobilized to a bead functionalized with a functionality such as streptavidin. A variety of suitable beads are available, non-limiting examples of which include DYNABEADS MYONE carboxylic acid magnetic beads (1 μm diameter), 1 μm PIERCE streptavidin magnetic beads, 4.5 μm DYNABEADS epoxy magnetic beads, 2.7 μm DYNABEADS epoxy magnetic beads, and 1 μm PIERCE NHS-activated magnetic beads. In some embodiments, during reporter immobilization onto beads functionalized with streptavidin, the reporter may be present at a concentration of about 0.1 nM to about 1,000 nM, for example within a range of about 0.1 nM to about 10 nM. In some embodiments, during reporter immobilization, the reporter is present at a concentration of about 0.1 nM to about 1 nM, about 0.1 nM to about 10 nM, about 0.1 nM to about 100 nM, about 0.1 nM to about 250 nM, about 0.1 nM to about 500 nM, about 0.1 nM to about 1,000 nM, about 1 nM to about 10 nM, about 1 nM to about 100 nM, about 1 nM to about 250 nM, about 1 nM to about 500 nM, about 1 nM to about 1,000 nM, about 10 nM to about 100 nM, about 10 nM to about 250 nM, about 10 nM to about 500 nM, about 10 nM to about 1,000 nM, about 100 nM to about 250 nM, about 100 nM to about 500 nM, about 100 nM to about 1,000 nM, about 250 nM to about 500 nM, about 250 nM to about 1,000 nM, or about 500 nM to about 1,000 nM. In some embodiments, during reporter immobilization, the reporter is present at a concentration of about 0.1 nM, about 1 nM, about 10 nM, about 100 nM, about 250 nM, about 500 nM, or about 1,000 nM. In some embodiments, during reporter immobilization, the reporter is present at a concentration of at least about 0.1 nM, about 1 nM, about 10 nM, about 100 nM, about 250 nM, or about 500 nM. In some embodiments, during reporter immobilization, the reporter is present at a concentration of at most about 1 nM, about 10 nM, about 100 nM, about 250 nM, about 500 nM, or about 1,000 nM.

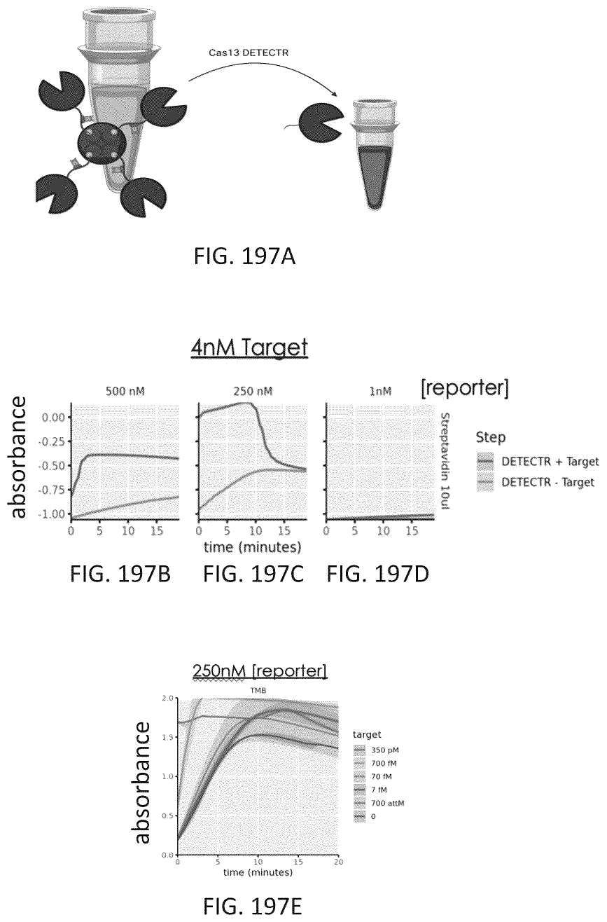

FIGS. 197A-197E show an illustrative programmable nuclease-based detection (e.g., DETECTR) assay scheme utilizing reporters comprising an enzyme-based detection moiety as well as assay results showing clear differentiation between target and non-target containing samples. FIG. 197A shows a schematic of an exemplary immobilized HRP-based reporter on a streptavidin bead being cleaved by an activated programmable nuclease, a Cas13 variant (SEQ ID NO: 21) in this case, thereby generating a visual signal upon introduction of an HRP-substrate such as 3,3′,5,5′-tetramethylbenzidine (TMB) as described herein. FIGS. 197B-197D show absorbance vs time plots for three reactions, where reporters were immobilized onto 10 μL of streptavidin-functionalized beads at reporter concentrations of 500 nM, 250 nM, and 1 nM, respectively. After running the DETECTR reaction, the beads were immobilized and the supernatant was added to the HRP-substrate TMG before absorbance at 450 nm was measured. In each of the three cases, the top trace shows 450 nm absorbance values for when the target was present at a concentration of 4 nM. The bottom trace shows absorbance values for the no target control (NTC). The maximum absorbance signal under the conditions tested can be observed in FIG. 197C, where the reporter was immobilized at 250 nM. FIG. 197E shows absorbance versus time when 250 nM of reporter was used to generate the streptavidin beads and the target concentration was varied from 0 to 350 μM in concentration. Each reporter molecule included a single HRP enzyme, a linker, and a biotin functionality for conjugation to a bead. Bead preparation allowed for conjugation of multiple reporters to a single bead. The beads were magnetic beads to facilitate separation of supernatant and un-cleaved reporters. Under these assay conditions, the limit of detection (LOD) was estimated to be at least about 700 fM.

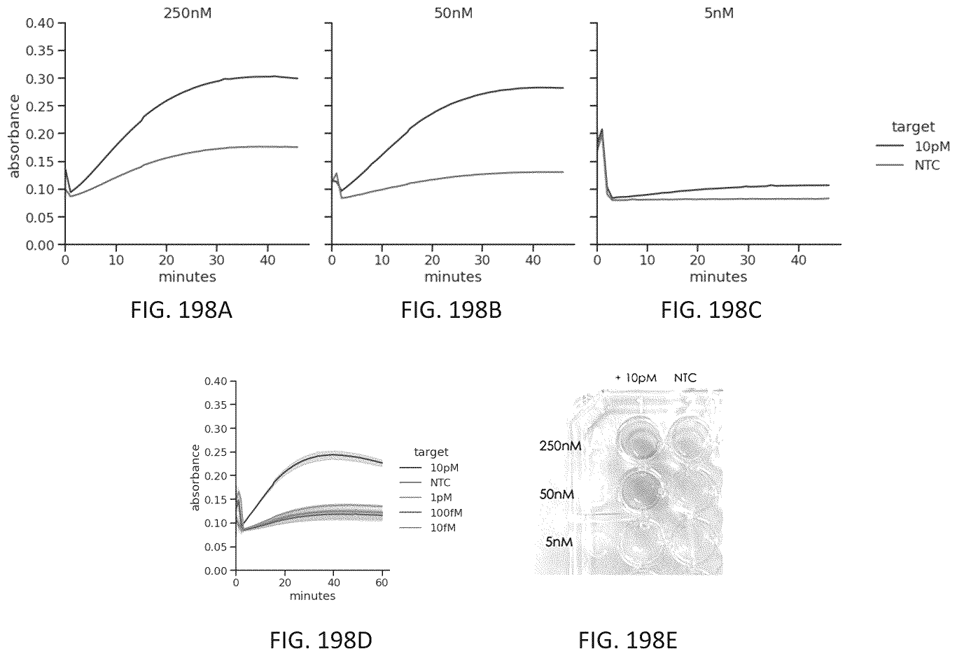

FIGS. 198A-198E show results for a study involving effects of different concentrations of reporter functionalized to a streptavidin-coated plate, and measurements to determine the limit of detection. FIGS. 198A-198C show the effects of varying the reporter concentration functionalized to a streptavidin-coated plate. The DETECTR assay performed was substantially the same as that of FIGS. 197A-197E, but with the reporter immobilized to a streptavidin-coated plate instead of a magnetic bead. The top trace depicts absorbance values for when target is present at 10 μM. The bottom trace depicts absorbance values for when target is not present, or no target control (NTC). FIG. 198D shows the results of a study where the target concentration was varied from 10 fM to 10 μM. From this data, the limit of detection for the target was determined to be at least 10 μM when 50 nM reporter was bound to the plate under the conditions tested. FIG. 198E shows an image of a multiwell plate corresponding to the data plotted in FIGS. 198A-198C, where the left column shows wells containing 10 PM target and the right column shows wells containing no target. The top row contains 250 nM reporter, the middle row contains 50 nM reporter, and the bottom row contains 5 nM reporter.

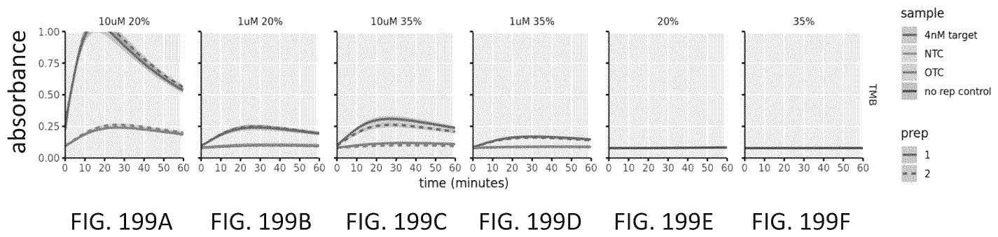

Described herein are various embodiments of DETECTR assays that may utilize hydrogels as a support for reporters, as described herein. In some embodiments, such hydrogels may comprise poly (ethylene glycol) diacrylate, or equivalently PEGDA or PEG-DA. FIGS. 199A-199F show absorbances versus time results for HRP-hydrogels at different PEG-DA concentrations (20% or 35% PEG-diacrylate (MW=700 monomers) with 80% or 65% unfunctionalized PEG (MW=600 monomers) respectively) co-polymerized with different concentrations of reporters (10 uM or 1 uM) comprising HRP-based detection moieties and acrydite functionalities (rep188). The hydrogels served as a solid support for the reporters comprising HRP for signal enhancement. The particular hydrogel constructions of this example is a non-limiting example. Alternative compositions and constructions are provided herein.

In some embodiments, reporters configured for HRP-reporter-based programmable nuclease-based detection (e.g., DETECTR) assays may be immobilized onto a solid support as described herein. In some embodiments, one or more HRP-conjugated reporters may be immobilized onto a solid support comprising a resin. In some embodiments, one or more HRP-conjugated reporters may be immobilized onto a solid support comprising an activated resin. In some embodiments, one or more HRP-conjugated reporters may be immobilized onto a solid support comprising a resin activated with N-Hydroxysuccinimide (NHS) functionalities. In some embodiments, one or more HRP-conjugated reporters are immobilized onto a solid support comprising a resin activated with NHS functionalities, wherein the reporters comprise corresponding amine functionalities as described herein. In some embodiments, one or more HRP-conjugated reporters are immobilized onto a solid support comprising a resin activated with NHS functionalities, wherein the reporters comprise a protein, for example an enzyme such as HRP. In some embodiments, one or more reporters are configured for HRP-based DETECTR assays and may be immobilized onto one or more solid support comprising a resin activated with NHS functionalities, wherein the reporters comprise an HRP and an amine functionality connected by a linker as described herein. In some embodiments, the resin may comprise agarose.

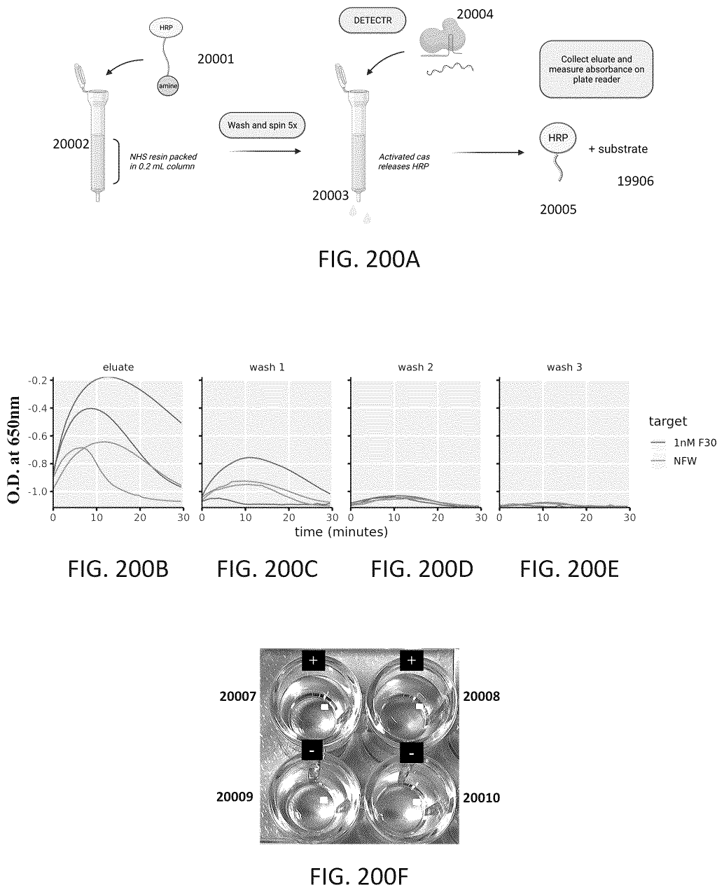

FIGS. 200A-200F show a schematic and results for a programmable nuclease-based detection assay utilizing NHS-activated resin as a solid support for an HRP-based reporter for signal enhancement (rep190). FIG. 200A shows a schematic describing the immobilization of an HRP-reporter and an HRP-based detection assay. In FIG. 200A a reporter, 20001, comprising HRP and an amine functionality is immobilized onto the NHS activated resin packed into a 0.2 mL column 20002. The column may then be washed and exposed to programmable-nuclease based detection (e.g., DETECTR) reagents 20004 comprising a programmable nuclease (e.g., SEQ ID NO: 34) complexed with a guide nucleic acid, as described herein. In the last step of FIG. 200A, the eluate, which contains cleaved reporter fragments comprising HRP 20005 when the target is present, may be mixed with a substrate (e.g., TMB) for generation of a detectable signal (e.g., a color change which can be detected by absorbance measurements) as described herein.

FIG. 200B shows assay results in the form of absorbance (O.D. at 650 nm) versus time, for the assay eluate comprising a substrate and the cleaved HRP-based reporter, rep 190, as described herein. FIGS. 200C-200E show measurements of the eluates from a first wash, a second wash and a third wash, respectively, of the resin packed column. In FIG. 200B, the two top traces show results for the assay carried out with 1 nM target, and the two bottom traces show results for the NEW condition. In FIG. 200C, the NEW condition is represented by the two middle traces. In FIGS. 200D-200E, traces for the two conditions overlapped. Additional details concerning the illustrative assays are described in the examples below.

In some embodiments, reporters configured for poly enzyme reporter-based DETECTR assays may be immobilized onto solid-supports. In some embodiments, reporters configured for poly HRP reporter-based DETECTR assays may be immobilized onto solid-supports comprising beads. In some embodiments, the beads are functionalized with streptavidin or NHS. In some embodiments, the poly HRP-reporters are immobilized onto the beads at a concentration of about 0.01 nM to about 0.5 μM, such as about 0.1 nM to about 250 nM, about 0.5 nM to about 100 nM, or about 1 nM to about 50 nM. In some embodiments, poly-HRP reporter molecules are immobilized at a concentration of about 10 nM. In some embodiments, mono-HRP reporter molecules are immobilized at a concentration of about 1 nM to about 50 μM, about 10 nM to about 25 μM, about 50 nM to about 10 μM, or about 100 nM to about 5 μM. In some embodiments, mono-HRP reporter molecules are immobilized at a concentration of about 1 μM. In some embodiments, HRP-DETECTR reagents are added to the beads functionalized with reporters, wherein the % solid content (e.g., concentration) of the beads is about 1 to about 50 mg/mL (e.g., about 5 to about 25, or about 10 mg/mL). In some embodiments, the reporter-functionalized beads are used at a concentration of about 10 mg/mL.

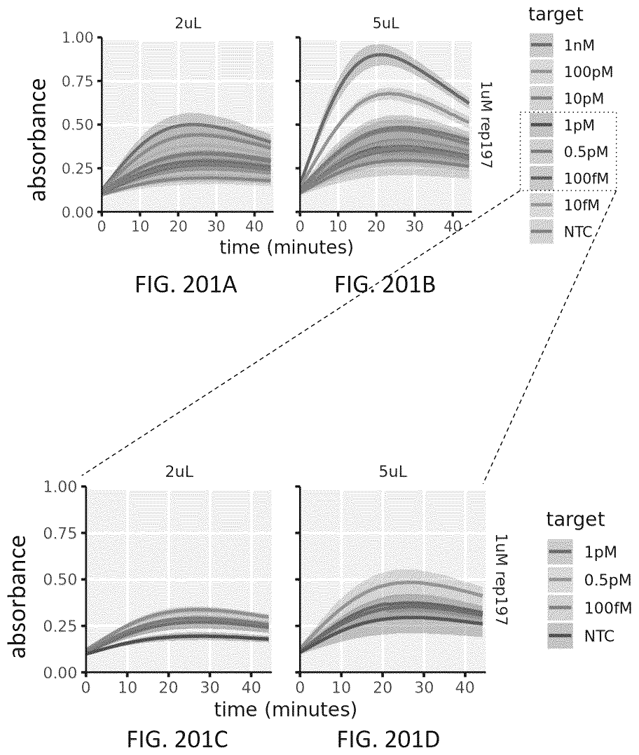

FIGS. 201A-201D show results of a poly-HRP-conjugated reporter-based programmable nuclease-based detection (e.g., DETECTR) assay study utilizing a poly-HRP reporter to determine a limit of detection (LOD) and to study the effect of varying the bead volume and concentration of the target. FIGS. 201A and 201B show absorbance values versus time for a series of target concentrations including 0 nM (NTC), 10 fM, 100 fM, 0.5 μM, 1 μM, 10 μM, 100 μM and 1 nM. Additionally, FIGS. 201A and 201B show DETECTR assay results using 2 μL of poly-HRP-reporter functionalized beads (as shown in FIGS. 196) and 5 μL of poly-HRP-reporter functionalized beads, respectively. In both cases, the concentration of the beads was 10 mg/mL. At the concentrations and amounts used, there were approximately 14×10⁹ reporter molecules per gram of beads. FIGS. 201C and 201D show the same absorbance data with only the 1 μM, 0.5 μM, 100 fM and NTC series. From this data, an LOD of about 100 fM was determined. There were three replicates per condition tested.

In various aspects, the present disclosure provides methods of using the compositions, devices, and systems disclosed herein to detect a target nucleic acid. In some embodiments, the method comprises (a) contacting the sample with a composition disclosed herein to produce a reaction fluid, wherein (i) the linker is conjugated to the support, (ii) the composition further comprises a guide nucleic acid configured to bind to the target nucleic acid, and (iii) the programmable nuclease cleaves the linker in response to formation of a complex comprising the programmable nuclease, the guide nucleic acid, and the target nucleic acid; (b) separating the reaction fluid from the support; (c) contacting the separated reaction fluid with a substrate of the one or more enzymes, wherein presence of the one or more enzymes in the fluid produces a detectable signal from the enzyme substrate; and (d) detecting the detectable signal. In some embodiments, the method comprises (a) contacting the sample with a composition disclosed herein to produce a reaction fluid, wherein (i) the composition further comprises a programmable nuclease and a guide nucleic acid, (ii) the guide nucleic acid is configured to bind to the target nucleic acid, and (iii) the programmable nuclease cleaves the linker in response to formation of a complex comprising the programmable nuclease, the guide nucleic acid, and the target nucleic acid; (b) contacting the reaction fluid with a plurality of beads, wherein each bead reacts with a plurality of the functionalities, thereby immobilizing the functionalities to the beads; (c) separating the reaction fluid from the beads; (d) contacting the separated reaction fluid with a substrate of the one or more enzymes, wherein presence of the one or more enzymes in the fluid produces a detectable signal from the enzyme substrate; and (e) detecting the detectable signal.

In some embodiments, the method further comprises concentrating the one or more enzymes prior to contacting the one or more enzymes with the substrate, wherein the concentrating comprises separating the one or more enzymes from at least a portion of the separated reaction fluid. For example, the enzyme may be captured to a support, such as a polymer matrix. The enzyme may be retained on the polymer matrix having a volume smaller than the reaction fluid applied to the matrix, thereby concentrating the enzyme. The enzyme may be reacted with a substrate while complexed to the matrix, or may be eluted in a fluid for subsequent reaction with the substrate. In embodiments where the enzyme is reacted to the substrate while complexed to the matrix, the substrate solutions comprising the substrate may be added serially (e.g., 2, 3, 4, 5, or more times), which may then be combined to detect enzymatic activity. In some embodiments, the enzyme comprises a horseradish peroxidase (HRP).

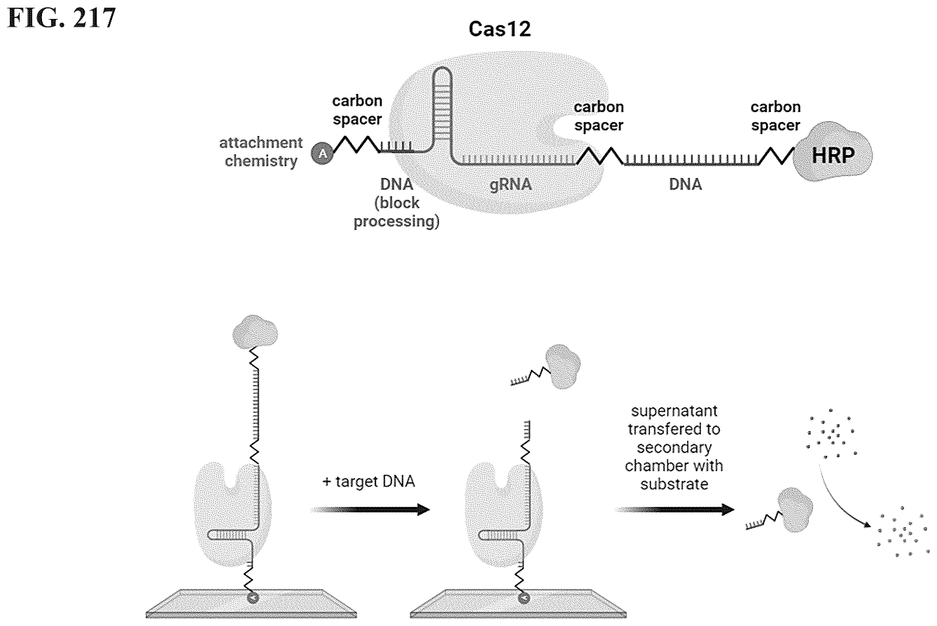

In some cases, the reporters are immobilized to the surface by attachment to the non-naturally occurring guide nucleic acid, a linkage immobilizing the guide nucleic acid to a surface, or both. In some instances, at least one of the reporters is immobilized to the surface by attachment to an end of the non-naturally occurring guide nucleic acid that is distal to the linkage. For example, at least one of the reporters can be immobilized by attaching to the 3′ end of a spacer region of the guide nucleic acid. In some cases, at least one of the reporters is immobilized to the surface by attachment to the linkage. For example, at least one of the reporters can be immobilized by attaching to a tether. In some embodiments, a plurality of reporters (e.g., at least 2, 3, 4, 5, 10, 15, or more) are attached to the linkage. In some embodiments, reporters are attached to both the linkage and an end of the guide nucleic acid. In some cases, a reporter comprises a detection moiety. In some cases, a reporter comprises an enzyme, such as HRP, as shown in FIG. 217. Molecules comprising a guide nucleic acid joined to a reporter are also referred to herein as “gREPs”.

Methods of Making Polymer Matrices with Immobilized Reporters

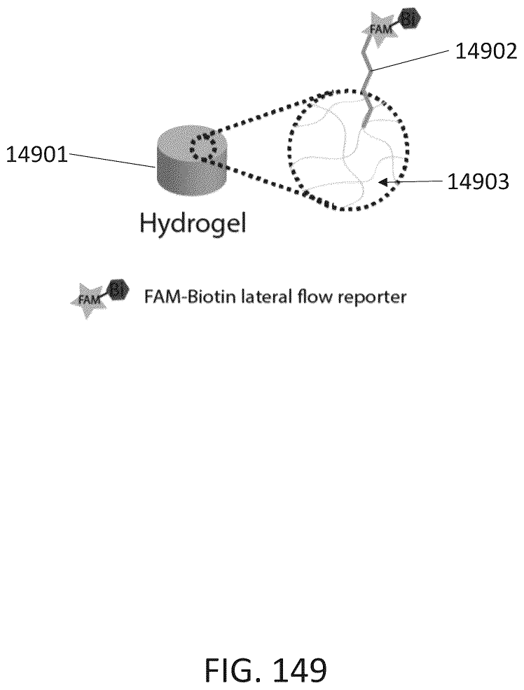

FIG. 149 shows an exemplary polymer immobilization matrix (14901) comprising a plurality of immobilized DETECTR reaction components. The DETECTR reaction components may comprise one or more reporters, one or more programmable nucleases, and/or one or more guide nucleic acids. In some embodiments, the polymer matrix may comprise a hydrogel. In the exemplary embodiment shown in FIG. 149, a plurality of reporters (14902) may be immobilized within a hydrogel (14901) matrix. In some embodiments, methods of immobilizing a reporter (14902) and/or other DETECTR reaction component may comprise (a) providing a polymerizable composition comprising: (i) a plurality of oligomers, (ii) a plurality of polymerizable (e.g., functionalized) oligomers, (iii) a set of polymerizable (e.g., functionalized) reporters (and/or other DETECTR reaction components), and (iv) a set of polymerization initiators; and (b) initiating the polymerization reaction by providing an initiation stimulus.

Co-polymerization of the reporter into the hydrogel may result in a higher density of reporter/unit volume or reporter/unit area than other immobilization methods utilizing surface immobilization (e.g., onto beads). Co-polymerization of the reporter into the hydrogel may result in less undesired release of the reporter (e.g., during an assay, a measurement, or on the shelf), and thus may cause less background signal, than other immobilization strategies (e.g., conjugation to a pre-formed hydrogel, bead, etc.). In at least some instances this may be due to better incorporation of reporters into the hydrogel as a co-polymer and fewer “free” reporter molecules retained on the hydrogel via non-covalent interactions or non-specific binding interactions.

In some embodiments, the plurality of oligomers and the plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for programmable nucleases to diffuse into the hydrogel and access internal reporter molecules. The relative percentages and/or molecular weights of the oligomers may be varied to vary the pore size of the hydrogel. For example, pore size may be tailored to increase the diffusion coefficient of the programmable nucleases.

In some embodiments, the functional groups attached to the reporters may be selected to preferentially incorporate the reporters into the hydrogel matrix via covalent binding at the functional group versus other locations along the nucleic acid of the reporter. In some embodiments, the functional groups attached to the reporters may be selected to favorably transfer free radicals from the functionalized ends of polymerizable oligomers to the functional group on the end of the reporter (e.g., 5′ end), thereby forming a covalent bond and immobilizing the reporter rather than destroying other parts of the reporter molecules.

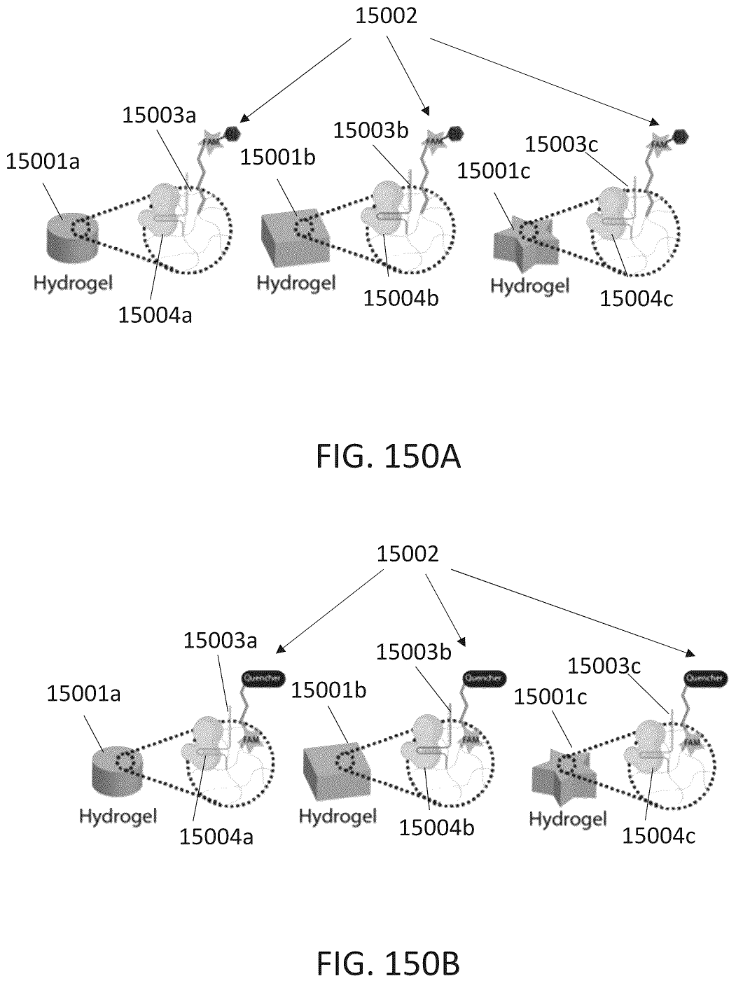

In some embodiments, the polymerizable composition may further comprise one or more polymerizable nucleic acids. In some embodiments, the polymerizable nucleic acids may comprise guide nucleic acids (e.g., guide nucleic acids 15003a, 15003b, or 15003c shown in FIGS. 150A-150B). In some embodiments, the polymerizable nucleic acids may comprise linker or tether nucleic acids. In some embodiments, the polymerizable nucleic acids may be configured to bind to a programmable nuclease (e.g., programmable nuclease 15004a, 15004b, or 15004c shown in FIGS. 150A-150B). In some embodiments, the programmable nuclease may be immobilized in the polymer matrix.

In some embodiments, the oligomers may form a polymer matrix comprising a hydrogel. In some embodiments, the oligomers may comprise poly (ethylene glycol) (PEG), poly (siloxane), poly (hydroxyethyl acrylate, poly (acrylic acid), poly (vinyl alcohol), poly (butyl acrylate), poly (2-ethylhexyl acrylate), poly (methyl acrylate), poly (ethyl acrylate), poly (acrylonitrile), poly (methyl methacrylate), poly (acrylamide), poly (TMPTA methacrylate), chitosan, alginate, or the like, or any combination thereof. One of ordinary skill in the art will recognize that the oligomers may comprise any oligomer or mix of oligomers capable of forming a hydrogel.

In some embodiments, the oligomers may comprise polar monomers, nonpolar monomers, protic monomers, aprotic monomers, solvophobic monomers, or solvophillic monomers, or any combination thereof.

In some embodiments, the oligomers may comprise a linear topology, branched topology, star topology, dendritic topology, hyperbranched topology, bottlebrush topology, ring topology, catenated topology, or any combination thereof. In some embodiments, the oligomers may comprise 3-armed topology, 4-armed topology, 5-armed topology, 6-armed topology, 7-armed topology, 8-armed topology, 9-armed topology, or 10-armed topology.

In some embodiments, the oligomers may comprise at least about 2 monomers, at least about 3 monomers, at least about 4 monomers, at least about 5 monomers, at least about 6 monomers, at least about 7 monomers, at least about 8 monomers, at least about 9 monomers, at least about 10 monomers, at least about 20 monomers, at least about 30 monomers, at least about 40 monomers, at least about 50 monomers, at least about 60 monomers, at least about 70 monomers, at least about 80 monomers, at least about 90 monomers, at least about 100 monomers, at least about 200 monomers, at least about 300 monomers, at least about 400 monomers, at least about 500 monomers, at least about 600 monomers, at least about 700 monomers, at least about 800 monomers, at least about 900 monomers, at least about 1000 monomers, at least about 2000 monomers, at least about 3000 monomers, at least about 4000 monomers, at least about 5000 monomers, at least about 6000 monomers, at least about 7000 monomers, at least about 8000 monomers, at least about 9000 monomers, at least about 10000 monomers, at least about 20000 monomers, at least about 30000 monomers, at least about 40000 monomers, at least about 50000 monomers, at least about 60000 monomers, at least about 70000 monomers, at least about 80000 monomers, at least about 90000 monomers, or at least about 100000 monomers.

In some embodiments, the oligomers may comprise a homopolymer, a copolymer, a random copolymer, a block copolymer, an alternative copolymer, a copolymer with regular repeating units, or any combination thereof.

In some embodiments, the oligomers may comprise 1 type of monomer, 2 types of monomers, 3 types of monomers, 4 types of monomers, 5 types of monomers, 6 types of monomers, 7 types of monomers, 8 types of monomers, 9 types of monomers, or 10 types of monomers.

The polymerizable oligomers may comprise any of the oligomers described herein. In some embodiments, the polymerizable oligomers may comprise one or more functional groups. In some embodiments, the functional group may comprise an acrylate group, N-hydroxysuccinimide ester group, thiol group, carboxyl group, azide group, alkyne group, an alkene group, or any combination thereof. One of ordinary skill in the art will recognize that a variety of functional groups may be used to functionalize oligomers into polymerizable oligomers depending on the desired properties of the polymerizable oligomers.

In some embodiments, the polymerizable oligomers may form a polymer matrix comprising a hydrogel. In some embodiments, the polymerizable oligomers may comprise PEG, poly (siloxane), poly (hydroxyethyl acrylate, poly (acrylic acid), poly (vinyl alcohol), or any combination thereof. One of ordinary skill in the art will recognize that the set of polymerizable oligomers may comprise any polymer capable of forming a hydrogel.

In some embodiments, the set of polymerizable oligomers comprises polar monomers, nonpolar monomers, protic monomers, aprotic monomers, solvophobic monomers, or solvophillic monomers.

In some embodiments, the set of polymerizable oligomers comprises a linear topology, branched topology, star topology, dendritic topology, hyperbranched topology, bottlebrush topology, ring topology, catenated topology, or any combination thereof. In some embodiments, the set of polymerizable oligomers comprises 3-armed topology, 4-armed topology, 5-armed topology, 6-armed topology, 7-armed topology, 8-armed topology, 9-armed topology, or 10-armed topology.

In some embodiments, the set of polymerizable oligomers comprises at least about 2 monomers, at least about 3 monomers, at least about 4 monomers, at least about 5 monomers, at least about 6 monomers, at least about 7 monomers, at least about 8 monomers, at least about 9 monomers, at least about 10 monomers, at least about 20 monomers, at least about 30 monomers, at least about 40 monomers, at least about 50 monomers, at least about 60 monomers, at least about 70 monomers, at least about 80 monomers, at least about 90 monomers, at least about 100 monomers, at least about 200 monomers, at least about 300 monomers, at least about 400 monomers, at least about 500 monomers, at least about 600 monomers, at least about 700 monomers, at least about 800 monomers, at least about 900 monomers, at least about 1000 monomers, at least about 2000 monomers, at least about 3000 monomers, at least about 4000 monomers, at least about 5000 monomers, at least about 6000 monomers, at least about 7000 monomers, at least about 8000 monomers, at least about 9000 monomers, at least about 10000 monomers, at least about 20000 monomers, at least about 30000 monomers, at least about 40000 monomers, at least about 50000 monomers, at least about 60000 monomers, at least about 70000 monomers, at least about 80000 monomers, at least about 90000 monomers, or at least about 100000 monomers. As used herein, “about” may mean plus or minus 1 monomer, plus or minus 10 monomers, plus or minus 100 monomers, plus or minus 1000 monomers, plus or minus 10000 monomers, or plus or minus 100000 monomers.

In some embodiments, the set of polymerizable oligomers comprises a homopolymer, a copolymer, a random copolymer, a block copolymer, an alternative copolymer, a copolymer with regular repeating units, or any combination thereof.

In some embodiments, the set of polymerizable oligomers comprises 1 type of monomer, 2 types of monomers, 3 types of monomers, 4 types of monomers, 5 types of monomers, 6 types of monomers, 7 types of monomers, 8 types of monomers, 9 types of monomers, or 10 types of monomers.

In some embodiments, the polymerizable composition may comprise a mix of unfunctionalized or unmodified oligomers and polymerizable oligomers as described herein. In some embodiments, the unfunctionalized or unmodified oligomers may act as porogens to generate pores within the polymer matrix.

The polymerizable reporters may comprise any of the reporters described herein. In some embodiments, the set of polymerizable reporters may comprise one or more functional groups. In some embodiments, the functional group may comprise a single stranded nucleic acid, a double stranded nucleic acid, an acrydite group, a 5′ thiol modifier, a 3′ thiol modifier, an amine group, a I-Linker™ group, methacryl group, or any combination thereof. One of ordinary skill in the art will recognize that a variety of functional groups may be used with the set of polymerizable reporters depending on the desired properties of the polymerizable reporters.

In some embodiments, the set of initiators may comprise one or more photoinitiators or thermal initiators. In some embodiments, the set of initiators may comprise cationic initiators, anionic initiators, or radical initiators. In some embodiments, the set of initiators may comprise AIBN, AMBN, ADVN, ACVA, dimethyl 2,2′-azo-bis (2methylpropionate), AAPH, 2,2′-azobis [2-(2-imidazolin-2-yl)-propane] dihydrochloride, TBHP, cumene hydroperoxide, di-tert-butyl peroxide, dicumyl peroxide, BPO, dicyandamide, cyclohexyl tosylate, diphenyl (methyl) sulfonium tetrafluoroborate, benzyl (4-hydroxyphenyl)-methylsulfonium hexafluoroantimonate, (4-hydroxyphenyl) methyl-(2-methylbenzyl) sulfonium hexafluoroantimonate, camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis (dimethylamino)-benzophenone, 4,4′-bis (diethylamino)-benzophenone, 4,4′-dichlorobenzophenone, 4-(p-tolylthio) benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone, 1-benzoylchclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylantraquinone, sodium anthraquinone-2-sulfonate monohydrate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′bis (2-chlorophenyl)-4,4′,5,5′-tetraphenyl-1,2′-biimidazole, diphenyl (2,4,6-trimethyl-benzoyl) phosphine oxide, phenylbis (2,4,6-trimethyl-benzoyl) phosphine oxide, lithium phenyl (2,4,6-trimethylbenzoyl) phosphinate, diphenyliodonium trifluoromethanesulfonate, diphenyliodonium hexafluorophosphate, diphenyliodonium hexafluoroarsenate, bis (4-tert-butylphenyl)-iodonium triflate, bis (4-tert-butylphenyl) iodonium hexafluorophosphate, 4-isopropyl-4′-methyl-diphenyliodonium tetrakis (pentafluorophenyl) borate, [4-[(2-hydroxytetradecyl)-oxy]phenyl]phenyliodonium hexafluoroantimonate, bis [4-(tert-butyl) phenyl]-iodonium tetra (nonafluoro-tert-butoxy) aluminate, cyclopropyldiphenylsulfonium tetrafluoroborate, triphenylsulfonium bromide, triphenylsulfonium tetrafluoroborate, tri-p-tolylsulfonium triflate, tri-p-tolylsulfonium hexafluorophosphate, 4-nitrobenzenediazonium tetrafluoroborate, 2-(4-methoxyphenyl)-4,6-bis (trichloromethyl)-1,3,5-triazine, 2-(1,3-benzodioxol-5-yl)-4,6-bis (trichloromethyl)-1,3,5-triazine, 2-(4-methoxystyryl)-4,6-bis (trichloromethyl)-1,3,5-triazine, 2-(3,4-dimethoxystyryl)-4,6-bis (trichloromethyl)-1,3,5-triazine, 2-[2-(Furan-2-yl) vinyl]-4,6-bis (trichloromethyl)-1,3,5-triazine, 2-[2-(5-methylfuran-2-yl) vinyl]-4,6-bis (trichloromethyl)-1,3,5-triazine, 2-(9-oxoxanthen-2-yl) proprionic acid 1,5,7-triazabicyclo[4.4.0]dec-5-ene salt, 2-(9-oxoxanthen-2-yl) proprionic acid 1,5-diazabicyclo[4.3.0]non-5-ene salt, 2-(9-oxoxanthen-2-yl) proprionic acid 1,8-diazabicyclo [5.4.0]-undec-7-ene salt, acetophenone O-benzoyloxime, 2-nitrobenzyl cyclohexylcarbamate, 1,2-bis (4-methoxyphenyl)-2-oxoethyl cyclohexylcarbamate, tert-amyl peroxybenzoate, 4,4-azobis (4-cyanovaleric acid), 1,1′-azobis (cyclohexanecarbonitrile), 2,2′-azobisisobutyronitrile, benzoyl peroxide, 2,2-bi (tert-butylperoxy) butane, 1,1-bis (tert-butylperoxy) cyclohexane, 2,5-bis (tert-butylperoxy)-2,5-dimethylhexane, bis (1-(tert-butylperoxy)-1-methylethyl) benzene, 1,1-bis (tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, 2-Hydroxy-2-methylpropiophenone, or any combination thereof. One of ordinary skill in the art will recognize that a variety of initiators may be used depending on the desired reaction conditions and chemistries.

In some embodiments, the initiation stimulus is UV light. In some embodiments, the initiation stimulus is UV light through a photomask. In some embodiments, the initiation stimulus is heat.

In some embodiments, the hydrogel may comprise a circular cross-sectional shape, a rectangular cross-sectional shape, a star cross-sectional shape, a dollop shape, an amorphous shape, or any shape of interest, or any combination thereof (e.g., as shown in FIGS. 150A-150B).

In some embodiments, a mask may be used to shape the initiation stimulus deposition on the polymerizable components (e.g., oligomers, etc.) and thereby shape the resulting polymer matrix. In some embodiments, the mask may comprise a circular shape, a rectangular shape, a star shape, a dollop shape, an amorphous shape, or any shape of interest, or any combination thereof.

Hydrogel Compositions with Immobilized Reporters

FIG. 149 and FIGS. 150A-150B show examples of hydrogels comprising immobilized reporters. In some aspects, provided herein are compositions comprising a hydrogel (14901) comprising (a) a network of covalently bound oligomers (14903) and (b) immobilized reporters (14902) covalently bound to said network (14903).

FIG. 149 shows an exemplary hydrogel (14901) comprising a plurality of reporters (14902) co-polymerized with a plurality of oligomers (modified and unmodified) to form a network or matrix (14903). FIGS. 150A-150B show exemplary multiplexing schemes utilizing hydrogel-immobilized reporters which may be implemented in any of the devices or methods described herein. Multiplexing could be distinguished through spatial multiplexing by knowing the location of hydrogels functionalized with each guide nucleic acid and/or through shape, by using different shapes of hydrogel for each guide nucleic acid.

In some embodiments, the composition may comprise a hydrogel (15001) comprising (a) a polymer network comprising covalently bound oligomers co-polymerized with reporters (15002) to covalently bind and immobilize the reporters to said network, and (b) immobilized programmable nuclease complexes covalently bound to said network (e.g., via co-polymerization or after reporter-immobilized polymer formation), wherein said programmable nuclease complexes may comprise a programmable nuclease (15004) and a guide nucleic acid (15003). In some embodiments, the guide nucleic acid (15003) and/or the programmable nuclease (15004) may be immobilized to or in the hydrogel as described herein (e.g., during or after formation of the hydrogel).

In some embodiments, the network of covalently bound oligomers may comprise a network formed by polymerizing one or more PEG species. In some embodiments, the network of covalently bound oligomers may comprise a network formed by polymerizing PEG comprising acrylate functional groups. In some embodiments, the acrylate functional groups may be PEG end groups. In some embodiments, the network may be formed by polymerizing PEG comprising acrylate functional groups with unmodified PEG. The molecular weight of the acrylate-modified PEG (e.g., PEG-diacrylate) and the unmodified PEG may be the same or different.

In some embodiments, the network of covalently bound oligomers may comprise a network formed from polymerizing one or more PEG species, wherein each PEG species may comprise a linear topology, branched topology, star topology, dendritic topology, hyperbranched topology, bottlebrush topology, ring topology, catenated topology, or any combination thereof. In some embodiments, the network of covalently bound oligomers may comprise a network formed from polymerizing one or more PEG species comprising a 3-armed topology, a 4-armed topology, a 5-armed topology, a 6-armed topology, a 7-armed topology, a 8-armed topology, a 9-armed topology, or a 10-armed topology.

In some embodiments, the immobilized reporter may comprise a reporter molecule covalently bound to a linker molecule, wherein the linker molecule is covalently bound to the hydrogel (e.g., via co-polymerization with the oligomers as described herein). In some embodiments, the linker molecule may comprise a single stranded nucleic acid, a double stranded nucleic acid, an acrydite group, a 5′ thiol modifier, a 3′ thiol modifier, an amine group, a I-Linker™ group, or any combination thereof. One of ordinary skill in the art will recognize that a variety of linker molecules may be used.

In some cases, the immobilized guide nucleic acid may comprise a guide nucleic acid covalently bound to a linker molecule, wherein the linker molecule is covalently bound to the hydrogel. In some embodiments, the linker molecule may comprise a single stranded nucleic acid, a double stranded nucleic acid, an acrydite group, a 5′ thiol modifier, a 3′ thiol modifier, an amine group, a I-Linker™ group, or any combination thereof. One of ordinary skill in the art will recognize that a variety of linker molecules may be used.

In some cases, the immobilized programmable nuclease may comprise a programmable nuclease covalently bound to a linker molecule, wherein the linker molecule is covalently bound to the hydrogel. In some embodiments, the linker molecule may comprise a single stranded nucleic acid, a double stranded nucleic acid, an acrydite group, a 5′ thiol modifier, a 3′ thiol modifier, an amine group, a I-Linker™ group, or any combination thereof. One of ordinary skill in the art will recognize that a variety of linker molecules may be used.

Methods of Using Hydrogels with Immobilized Reporters

Any of the methods described herein may utilize hydrogels (14901) with immobilized reporters (14902) for target detection assays. In some embodiments, the hydrogel (14901) comprises (a) a network of covalently bound oligomers (14903) and (b) immobilized reporters (14902) covalently bound to said network (14903) as shown in FIG. 149. A solution comprising target nucleic acid molecules and programmable nuclease complexes may be applied to the hydrogel (e.g., by pipetting or flowing over the hydrogel). The immobilized reporters (14902) may comprise a nucleic acid with a sequence cleavable by the programmable complex when the programmable nuclease complex is activated by binding of its associated guide nucleic acid to a target nucleic acid molecule as described herein. When activated, the programmable nuclease complex may trans-cleave the cleavable nucleic acid of the reporter molecule and generates a detectable signal as described herein. For example, the reporter may comprise a detection moiety which may be release upon cleavage of the reporter as described herein. The detection moiety may comprise FAM-biotin which may be captured by one or more capture molecules coupled to a surface of a support (e.g., a lateral flow assay strip) at a detection location as described herein. Detection of the detectable signal generated at the detection location by the detection moiety may indicate the presence of the target nucleic acid in the sample as described herein.

Any of the multiplexing methods described herein may utilize hydrogels (15001a, 15001b, 15001c, etc.) with immobilized reporters (15002) for multiplexed target detection assays. In some embodiments, each hydrogel (15001a, 150001b, 15001c, etc.) may comprise (a) a polymer network of covalently bound oligomers co-polymerized with reporters (15002) to covalently bind and immobilize the reporters to said network, and (b) one or more immobilized programmable nuclease complexes covalently bound to said network as shown in FIGS. 150A-150B. Each of the programmable nuclease complexes may comprise a programmable nuclease (15004a, 15004b, 15004c, etc.) and a guide nucleic acid (15003a, 15003b, 15003c, etc.). In some embodiments, the guide nucleic acid (15003) and/or the programmable nuclease (15004) may be immobilized to or in the hydrogel as described herein (e.g., during or after formation of the hydrogel). In some embodiments, multiplexing for a plurality of different targets may be facilitated by providing a plurality different and/or spatially separated hydrogels comprising a plurality of different DETECTR reaction components. In some embodiments, each hydrogel may comprise a different programmable nuclease as described herein. Alternatively, or in combination, each hydrogel may comprise a different guide nucleic acid configured to bind to a different target nucleic acid sequence as described herein. Alternatively, or in combination, each hydrogel may comprise a different reporter as described herein. Alternatively, or in combination, each hydrogel may comprise a different shape and be deposited on a surface of a support at different detection locations. For example, as shown in FIGS. 150A-150B, a first hydrogel (15001a) may comprise a first programmable nuclease (15004a), a first guide nucleic acid (15003a) configured to bind a first target nucleic acid, and a first reporter (15002). A second hydrogel (15001b) may comprise a second programmable nuclease (15004b), a second guide nucleic acid (15003b) configured to bind a second target nucleic acid, and a second reporter (15002). A third hydrogel (15001c) may comprise a third programmable nuclease (15004c), a third guide nucleic acid (15003c) configured to bind a third target nucleic acid, and a third reporter (15002). The programmable nucleases (15004a, 15004b, 15004c) may be the same programmable nuclease or different programmable nuclease. The guide nucleic acids (15003a, 15003b, 15003c) may be different guide nucleic acids configured to recognize different target nucleic acids. The reporters (15002) may be the same reporter or different reporters. A solution comprising one or more target nucleic acid molecules may be applied to the hydrogels (15001a, 15002b, 15003c), e.g., by pipetting or flowing over the hydrogels. The immobilized reporters (15002) may comprise a nucleic acid with a sequence cleavable by the programmable nuclease complexes (15004a, 15004b, 15004c) when the programmable nuclease complexes are activated by binding of their respective guide nucleic acids (15003a, 15003b, 15003c) to their respective target nucleic acid molecules as described herein. When activated, the programmable nuclease complexes may trans-cleave the cleavable nucleic acid of the reporter molecule and generates a detectable signal at the detection location as described herein. For example, the reporter may comprise a detection moiety which may be release upon cleavage of the reporter as described herein. The detection moiety may comprise FAM-biotin as shown in FIG. 150A which may be captured by one or more capture molecules coupled to a surface of a support (e.g., a lateral flow assay strip) at a detection location as described herein. Alternatively, the detection moiety may comprise a quencher moiety which may be released from the hydrogel upon cleavage of the reporter, thereby allowing a fluorescent moiety on the other end of the reporter to fluoresce at the detection location comprising the hydrogel as shown in FIG. 150B. Detection of the detectable signal generated at the detection locations by the detection moiety may indicate the presence of the target nucleic acid in the sample as described herein. Each hydrogel (15001a, 15001b, 15001c) may have a different shape and detection of a target nucleic acid may comprise detecting a particular fluorescent shape corresponding to the hydrogel shape at the detection location.

Devices Comprising Hydrogels with Immobilized Reporters

Any of the systems or devices described herein may comprise one or more hydrogels with immobilized reporters.

In some embodiments, the systems and devices described herein may comprise a plurality of hydrogels each comprising reporter molecules (e.g., in order to facilitate multiplexing and/or improve signal). In some embodiments, a first hydrogel may comprise a shape different from a shape of a second hydrogel. In some embodiments, the first hydrogel may comprise a plurality of first reporter molecules different from a plurality of second reporter molecules of the second hydrogel. In some embodiments, the reporters are the same in the first and second hydrogels. In some embodiments, the first hydrogel may comprise a circular shape, a square shape, a star shape, or any other shape distinguishable from a shape of the second hydrogel. In some embodiments, the plurality of first reporter molecules may each comprise a sequence cleavable by a programmable nuclease complex comprising a first programmable nuclease and a first guide nucleic acid. In some embodiments, the plurality of second reporter molecules may each comprise a sequence not cleavable by the first programmable nuclease complex.

Any of the systems or devices described herein may comprise a plurality of hydrogels each comprising reporter molecules. For example, a first hydrogel may comprise a plurality of first reporter molecules different from a plurality of second reporter molecules of a second hydrogel. In some embodiments, the plurality of first reporter molecules may each comprise a first fluorescent moiety, wherein the first fluorescent moiety is different than second fluorescent moieties of in each of the plurality of second reporter molecules. In some embodiments, the plurality of first reporter molecules may each comprise a sequence cleavable by a first programmable nuclease complex comprising a first programmable nuclease and a first guide nucleic acid. In some embodiments, the plurality of second reporter molecules may each comprise a sequence cleavable by a second programmable nuclease complex comprising a second programmable nuclease and a second guide nucleic acid.

Any of the systems or devices described herein may comprise at least about 2 hydrogels, at least about 3 hydrogels, at least about 4 hydrogels, at least about 5 hydrogels, at least about 6 hydrogels, at least about 7 hydrogels, at least about 8 hydrogels, at least about 9 hydrogels, at least about 10 hydrogels, at least about 20 hydrogels, at least about 30 hydrogels, at least about 40 hydrogels, at least about 50 hydrogels, at least about 60 hydrogels, at least about 70 hydrogels, at least about 80 hydrogels, at least about 90 hydrogels, at least about 100 hydrogels, at least about 200 hydrogels, at least about 300 hydrogels, at least about 400 hydrogels, at least about 500 hydrogels, at least about 600 hydrogels, at least about 700 hydrogels, at least about 800 hydrogels, at least about 900 hydrogels, at least about 1000 hydrogels,

Any of the systems or devices described herein may comprise one or more compartments, chambers, channels, or locations comprising the one or more hydrogels. In some embodiments, two or more of the compartments may be in fluid communication, optical communication, thermal communication, or any combination thereof with one another. In some embodiments, two or more compartments may be arranged in a sequence. In some embodiments, two or more compartments may be arranged in parallel. In some embodiments, two or more compartments may be arranged in sequence, parallel, or both. In some embodiments, one or more compartments may comprise a well. In some embodiments, one or more compartments may comprise a flow strip. In some embodiments, one or more compartments may comprise a heating element.

In some embodiments, the device may be a handheld device. In some embodiments, the device may be point-of-need device. In some embodiments, the device may comprise any one of the device configurations described herein. In some embodiments, the device may comprise one or more parts of any one of the device configurations described herein.

### Amplifying Signals Using Positive Feedback Systems

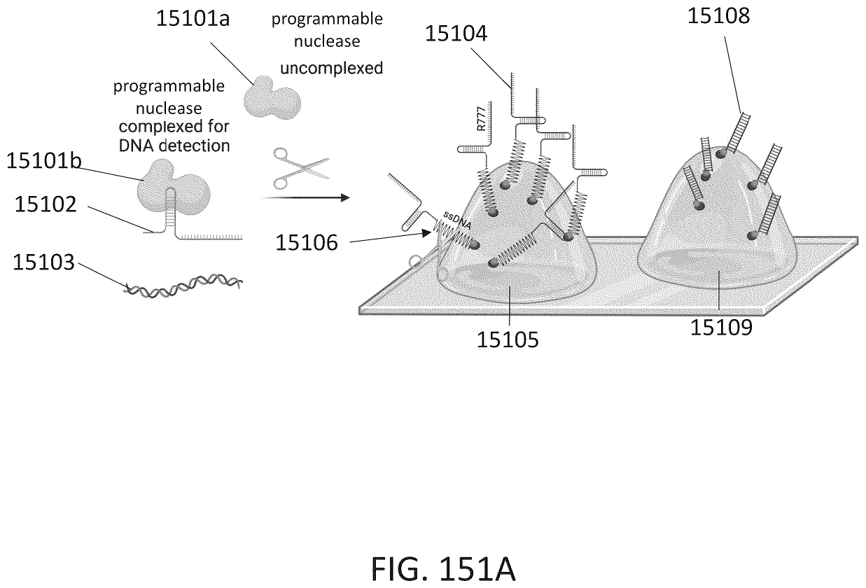

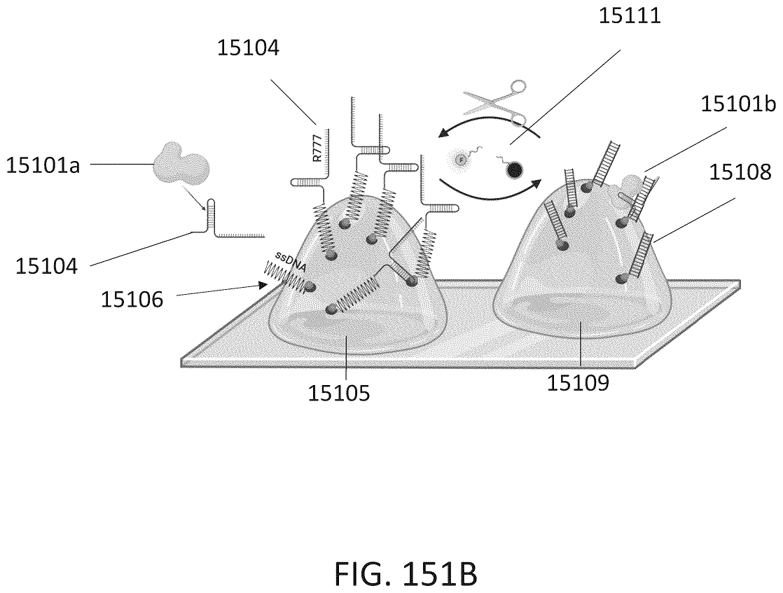

Any of the methods described herein may comprise amplifying a detection signal using a positive feedback system. FIGS. 151A-151B illustrates an exemplary positive feedback system for signal amplification. In some embodiments, a method for signal amplification may comprise binding a first nuclease, e.g., a first programmable nuclease (15101a) bound to a first guide nucleic acid (15102) with a first target nucleic acid (15103) to generate a first activated programmable nuclease complex (15101b), as shown in FIG. 151A. The first target nucleic acid (15103) may be present in a sample. Activation of the first programmable nuclease may result in release of one or more secondary target-specific guide nucleic acids (15104) from a first location (15105). The secondary target-specific guide nucleic acids (15104) may each comprise a nucleic acid tether (15106) capable of being cleaved by the first activated programmable nuclease complex. The secondary target-specific guide nucleic acids may be released by trans-cleaving the nucleic acid tethers (15106) via the first activated programmable nuclease complex. The secondary target-specific guide nucleic acids (15104) may then bind to an uncomplexed second programmable nuclease (15101a) present at the first location (15105), as shown in FIG. 151B. The second programmable nuclease (15101a) may then bind a second target nucleic acid (15108) at a second location (15109) to generate a second activated programmable nuclease complex (15101b). The second activated programmable nuclease complex (15101b) may then cleave the second target nucleic acid (15108) or remain immobilized at the second location (15109). One or more reporters may be present (e.g., free-floating, immobilized to a surface of a support at a third location, etc.) which may be cleaved by the first and second activated programmable nuclease complexes as described herein. Alternatively, or in combination, one or more of the secondary target-specific guide nucleic acid (15104), the tether (15106), and/or the second target nucleic acid (15108) may comprise a detection moiety which may provide a detectable signal upon cleavage of the nucleic acid species to which it is bound. In some embodiments, the detection moiety comprises a quencher-fluorophore pair as described herein.

A single first target nucleic acid can lead to the release of a plurality of secondary target-specific guide nucleic acids and the generation of the plurality of second activated programmable nuclease complexes as described herein. Then each second activated programmable nuclease complex can lead to the generation of another plurality of secondary target-specific guide nucleic acids and another plurality of second activated programmable nuclease complexes. And so on. The second activated programmable nuclease complexes can generate additional signal beyond that of the first activated programmable nuclease complex alone as described herein. Therefore, the detection of the single first target nucleic acid can activate a positive feedback loop for amplifying its signal.

In some aspects, provided herein are compositions for amplifying a detection signal using a positive feedback system. In some embodiments, a composition may comprise: (a) a first set of programmable nucleases (15101) each comprising a first guide nucleic acid (15102), wherein each programmable nuclease in the first set of programmable nucleases is configured to bind with a first target nucleic acid (15103) and then trans-cleave a plurality of nucleic acids comprising a first sequence; (b) a plurality of secondary target-specific guide nucleic acids (15104) each comprising a nucleic acid tether (15106) comprising the first sequence; (c) a second set of programmable nucleases (15101) each configured to bind with a secondary target-specific guide nucleic acid which is configured to bind with a second target nucleic acid and then cleave a plurality of nucleic acids comprising a second sequence; a plurality of second target nucleic acids (15108) each comprising the second sequence.

In some embodiments, the first programmable nuclease (15101) may be free in solution, as illustrated in FIGS. 151A-151B. In some embodiments, the first programmable nuclease (15401) may be immobilized to a substrate (e.g., 15105, 15109, etc.). In some embodiments, the second programmable nuclease (15101) may be free in solution. In some embodiments, the second programmable nuclease (15101) may be immobilized to a substrate (e.g., 15105, 15109, etc.).

In some embodiments, one or more components of the composition may be immobilized on a substrate (e.g., 15105, 15109).

In some embodiments, the substrate (15105, 15109) may comprise a hydrogel, as illustrated in FIG. 151. In some embodiments, a first hydrogel (15105) may comprises a secondary target-specific guide nucleic acid (15104) immobilized by a single-stranded nucleic acid (15106). In some embodiments, a second hydrogel (15109) may comprise an immobilized second target nucleic acid (15108). In some embodiments, the substrate (15105, 15109) may comprise a reporter-incorporated hydrogel as described herein. In some embodiments, the substrate (15105, 15109) may be in the form of a bead. In some embodiments, the substrate (15105, 15109) may be a glassy material. In some embodiments, the substrate (15105, 15109) may be a polymeric material.

In some embodiments, the secondary target-specific guide nucleic acids (15104) may be immobilized to a substrate (15105, 15109). In some embodiments, the secondary target-specific guide nucleic acids (15104) may be immobilized with a single stranded nucleic acid tether (15106). In some embodiments, the secondary target-specific guide nucleic acids (15104) may be free in solution. In some embodiments, the secondary target-specific guide nucleic acids (15104) may comprise reporters. In some embodiments, the secondary target-specific guide nucleic acids (15104) may comprise detection moieties (15111).

In some embodiments, the second target nucleic acids (15108) may be immobilized on a substrate (15105, 15109). In some embodiments, the second target nucleic acids (15108) may be free in solution. In some embodiments, the second target nucleic acids (15108) may comprise reporters. In some embodiments, the second target nucleic acids (15108) may comprise detection moieties (15111).

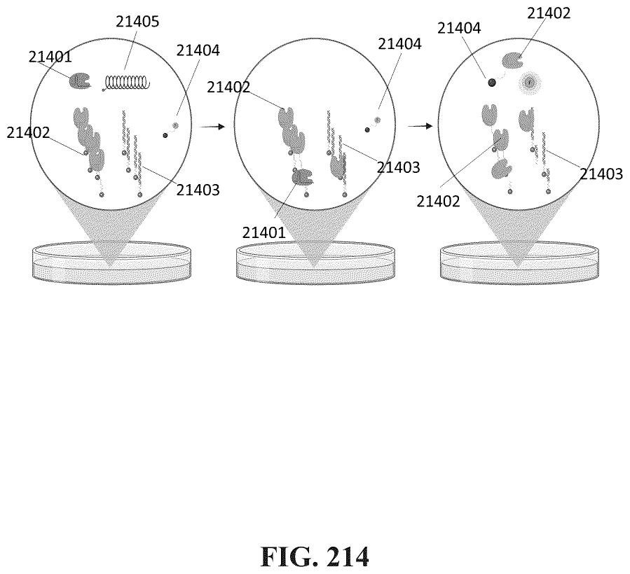

In some embodiments, the compositions for amplifying a detection signal using a positive feedback system can be described step-wise as shown in FIG. 214 which shows a schematic representation of a surface-immobilized positive-feedback system wherein the Cas #1 is initially in the solution and Cas #2 is initially immobilized. In a subsequent step, Cas #1 forms an activated complex upon binding to a first target nucleic acid and cleaves the ssDNA tether linking Cas #2 to the surface. The released Cas #2 is activated upon complexing with a second target nucleic acid (immobilized target #2), and the activated Cas #2 is effective to release additional immobilized Cas #2 and to cleave the reporter to produce a detectable product. In some embodiments, Cas #1 and Cas #2 are the same. In some embodiments, Cas #1 and Cas #2 are different. In some embodiments, target #1 is different for each of a plurality of reactions (e.g., in a plurality of wells), and target #2 is (a) different from each target #1, and (b) the same as each target #2 in the plurality of reactions.

The programmable nucleases may comprise any of the programmable nucleases described herein. In some embodiments, the nuclease is an endonuclease. In some embodiments, the nuclease is a Cas9 enzyme. In some embodiments, the nuclease is a mutant Cas9 enzyme. In some embodiments, the nuclease is an engineered Cas9 enzyme. In some embodiments, the nuclease is a Cas12 enzyme. In some embodiments, the nuclease is a Cas13 enzyme. In some embodiments, the nuclease is a Cas14 enzyme. In some embodiments, the nuclease is a CasPhi enzyme.

In some embodiments, the first and second programmable nucleases are the same. In some embodiments, the first and second programmable nucleases are different. In some embodiments, the programmable nucleases carry out cis cleavage. In some embodiments, the programmable nucleases carry out trans cleavage.

In some embodiments, cleaving by a programmable nuclease activates a reporter. In some embodiments, cleaving by a programmable nuclease activates (e.g., releases, unquenches, etc.) a detection moiety. In some embodiments, cleaving a nucleic acid tether activates a reporter. In some embodiments, cleaving a second target nucleic acid activates a reporter.

### Devices for Amplifying Signals Using Positive Feedback Systems

Any of the devices described herein may be configured for amplifying a detection signal using a positive feedback system. In some embodiments, a device may comprise one or more compartments configured to: (a) bind a first nuclease (15101) with a first guide nucleic acid (15102) and a first target nucleic acid (15103) to generate a first complex; (b) release one or more second guide nucleic acids (15104) each comprising a nucleic acid tether (15106) by cleaving the nucleic acid tether(s) (15106) with the first complex; (c) bind the second guide nucleic acids (15104) each with a second nuclease (15101) and a second target nucleic acid (15108) to generate a plurality of second complexes; (d) cleave a plurality of reporters with the first and second complexes as described herein. Additional second complexes may be formed by further cleavage by the first and second complexes as described herein.

In some embodiments, a device may comprise one or more compartments comprising: (a) a first set of nucleases (15101) each comprising a first guide nucleic acid (15402), wherein each nuclease in the first set of nucleases is configured to bind with a first target nucleic acid (15103) and then cleave a plurality of nucleic acids comprising a first sequence; (b) a plurality of secondary target-specific guide nucleic acids (15104)) each comprising a nucleic acid tether (15106) comprising the first sequence; (c) a second set of nucleases (15101) each configured to bind with the secondary target-specific guide nucleic acid (15104) and a second target nucleic acid (15108) and then cleave a plurality of nucleic acids; (d) a plurality of second target nucleic acids (15108).

In some embodiments, one or more compartments may be in fluid communication, optical communication, thermal communication, or any combination thereof with one another. In some embodiments, one or more compartments may be arranged in a sequence. In some embodiments, one or more compartments may be arranged in parallel. In some embodiments, one or more compartments may be arranged in sequence, parallel, or both. In some embodiments, one or more compartments may comprise a well. In some embodiments, one or more compartments may comprise a flow strip. In some embodiments, one or more compartments may comprise a heating element.

In some embodiments, the device may be a handheld device. In some embodiments, the device may be point-of-need device. In some embodiments, the device may comprise any one of the device configurations described in this disclosure. In some embodiments, the device may comprise one or more parts of any one of the device configurations described herein.

Assay Compositions with Thermostable Inorganic Pyrophosphatase

Any of the systems, methods, or devices described herein may comprise using thermostable inorganic pyrophosphatase (TIPP). In some embodiments, a composition for an assay may comprise TIPP. In some embodiments, the composition may comprise about 0.5 enzyme unit (U) per 10 μL of solution. In some embodiments, the composition may comprise at least about 0.1 U per 10 μL of solution. In some embodiments, the composition may comprise at most about 2 U per 10 μL of solution. In some embodiments, the composition may comprise at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 U per 10 μL of solution. In some embodiments, the composition may comprise at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 U per 10 μL of solution.

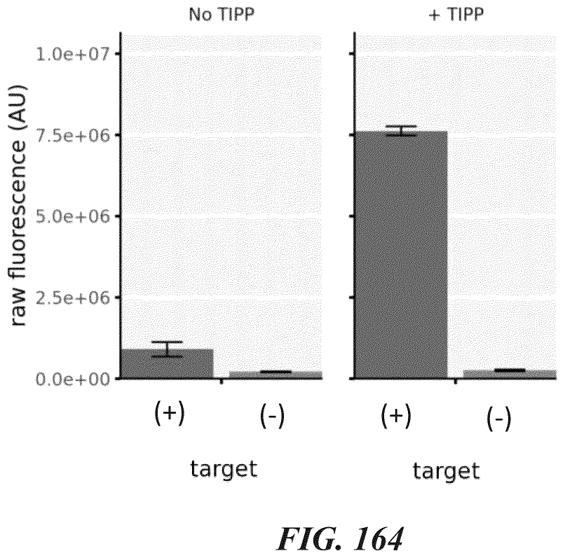

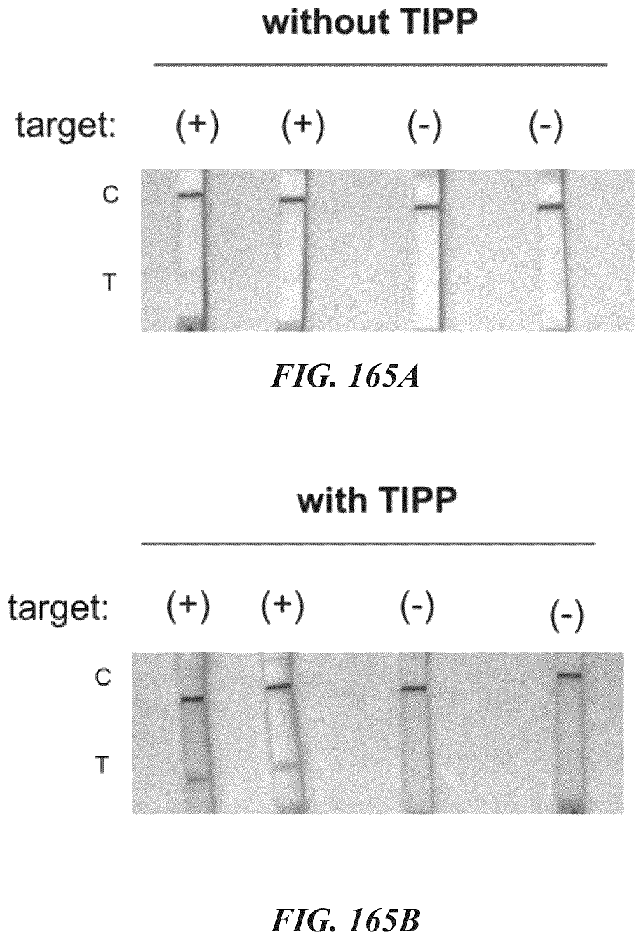

In some embodiments, TIPP may improve the signal to noise ratio of a programmable nuclease-based detection reaction. In some embodiments, TIPP may improve overall signal (e.g., fluorescence of a cleaved reporter as shown in FIG. 164 or intensity of a lateral flow assay strip detection location as shown in FIGS. 165A-165B). TIPP may improve signal by a factor, wherein the signal is indicative of the presence of a target nucleic acid. In some embodiments, the factor may be at least about 1.1. In some embodiments, the factor may be at least about 2. In some embodiments, the factor may be at least about 3. In some embodiments, the factor may be at least about 4. In some embodiments, the factor may be at least about 5. In some embodiments, the factor may be at least about 6. In some embodiments, the factor may be at least about 7. In some embodiments, the factor may be at least about 8. In some embodiments, the factor may be at least about 9. In some embodiments, the factor may be at least about 10.

In some cases, the TIPP enzyme may be present in the HotPot reaction mix at a concentration of 0.125 Units, 0.5 Units, 0.25 Units, 1.0 Units, 2.0 Units, 2.5 Units, or 4 Units per discrete reaction volume. In some cases, the buffer may comprise the TIPP signal enhancement reagent. Conversely, the TIPP signal enhancement reagent may be provided separately (e.g., in a separate vial in a kit) and added to a reaction mixture at a desired timepoint.

In some cases, a buffer may comprise a catalytic reagent for signal improvement or enhancement. In some cases, the catalytic reagent may enhance signal generation via hydrolysis of inorganic pyrophosphates. In some cases, the catalytic reagent may enhance signal generation via enhancement of DNA replication. In some cases, the catalytic reagent may enhance signal amplification via revival of Mg⁺² ions in the buffer solution which may otherwise be taken up by the phosphates produced from usage of dNTPs during the LAMP or NEAR reaction (or other isothermal or thermocycling replication reaction described herein). In some cases, the catalytic reagent may enhance signal generation by reviving the concentration of Mg+2 ions in the buffer thereby enhancing the function of the Cas nuclease effector enzyme. In some cases, the catalytic reagent for signal improvement may be an enzyme. In some cases, the catalytic reagent for signal improvement may be a Thermostable Inorganic Pyrophosphatase (TIPP).

### Nicking Enzyme Amplification Reaction (NEAR)

In some embodiments, a target nucleic acid may be amplified using a nicking enzyme amplification reaction (NEAR). NEAR may be used to amplify a region of a nucleic acid comprising a target nucleic acid. NEAR may comprise a forward primer and a reverse primer that at least partially anneals to complementary strands of a target nucleic acid 3′ of the region to be amplified. The forward primer and the reverse primer may comprise a stabilizing region that is not complementary to the target sequence. The forward primer and the reverse primer may comprise recognition regions that may be nicked by a nicking enzyme. A polymerase may polymerize a nucleic acid 5′ to 3′ from the 3′ end of the forward primer or the 3′ end of the reverse primer, using the strand to which the primer is annealed as a template, resulting in a double stranded nucleic acid product or amplicon. The newly synthesized strand may comprise a restriction site and may then serve as a template. The resulting double stranded nucleic acid amplicon may comprise nicking sites in both strands. A nicking enzyme may nick a single strand of the double stranded product or amplicon. The polymerase may polymerize a nucleic acid 5′ to 3′ from the 3′ end of the nucleic acid amplicon 5′ of the nick. The process may be repeated, thereby amplifying the target nucleic acid.

As described herein, a target nucleic acid may be detected using a DNA-activated programmable RNA nuclease (e.g., a Cas13), a DNA-activated programmable DNA nuclease (e.g., a Cas12), or an RNA-activated programmable RNA nuclease (e.g., a Cas13) and other reagents disclosed herein (e.g., RNA components). The target nucleic acid may be detected using DETECTR, as described herein. The target nucleic acid may be an RNA, reverse transcribed RNA, DNA, DNA amplicon, amplified DNA, synthetic nucleic acids, or nucleic acids found in biological or environmental samples. In some embodiments, the target nucleic acid is amplified prior to or concurrent with detection. In some embodiments, the target nucleic acid is reverse transcribed prior to amplification. The target nucleic acid may be amplified via NEAR of a target nucleic acid sequence. In some embodiments, the nucleic acid is amplified using NEAR coupled with reverse transcription (RT-NEAR). The NEAR amplification may be performed independently, or the NEAR amplification may be coupled to DETECTR for detection of the target nucleic acid. The RT-NEAR amplification may be performed independently, or the RT-NEAR amplification may be coupled to DETECTR for detection of the target nucleic acid. The DETECTR reaction may be performed using any method consistent with the methods disclosed herein.

NEAR may be performed as an isothermal reaction, for example NEAR may be performed at from about 37° C. to about 42° C. In some embodiments, NEAR may be performed at from about 15° C. to about 60° C., from about 15° C. to about 55° C., from about 15° C. to about 50° C., from about 15° C. to about 45° C., from about 15° C. to about 40° C., from about 15° C. to about 35° C., from about 15° C. to about 30° C., from about 15° C. to about 25° C., from about 15° C. to about 20° C., from about 20° C. to about 60° C., from about 20° C. to about 55° C., from about 20° C. to about 50° C., from about 20° C. to about 45° C., from about 20° C. to about 40° C., from about 20° C. to about 35° C., from about 20° C. to about 30° C., from about 20° C. to about 25° C., from about 25° C. to about 60° C., from about 25° C. to about 55° C., from about 25° C. to about 50° C., from about 25° C. to about 45° C., from about 25° C. to about 40° C., from about 25° C. to about 35° C., from about 25° C. to about 30° C., from about 30° C. to about 60° C., from about 30° C. to about 55° C., from about 30° C. to about 50° C., from about 30° C. to about 45° C., from about 30° C. to about 40° C., from about 30° C. to about 35° C., from about 35° C. to about 60° C., from about 35° C. to about 55° C., from about 35° C. to about 50° C., from about 35° C. to about 45° C., from about 35° C. to about 40° C., from about 40° C. to about 60° C., from about 40° C. to about 55° C., from about 40° C. to about 50° C., from about 40° C. to about 45° C., from about 45° C. to about 60° C., from about 45° C. to about 55° C., from about 45° C. to about 50° C., from about 50° C. to about 60° C., from about 50° C. to about 55° C., or from about 55° C. to about 60° C. In some embodiments, NEAR may be performed above about 15° C., above about 20° C., above about 25° C., above about 30° C., above about 35° C., above about 40° C., above about 45° C., or above about 50° C. In some embodiments, an SDA reaction may be performed below about 60° C., below about 55° C., below about 50° C., below about 45° C., below about 40° C., below about 35° C., below about 30° C., below about 25° C., below about 20° C., or below about 15° C. In some embodiments, NEAR may be performed at about room temperature. In some embodiments, a nucleic acid sample may be heated prior to isothermal amplification. In some embodiments, the nucleic acid sample heated prior to isothermal amplification may comprise one or more primers. The nucleic acid sample may be heated to about 95° C. prior to isothermal amplification. The nucleic acid sample may be heated to a temperature sufficient to dissociate two strands of a double stranded nucleic acid sequence.

NEAR may amplify a target nucleic acid to detectable levels within about 30 seconds, 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. NEAR may amplify a target nucleic acid to detectable levels within about 1 hour, about 1.1 hours, about 1.2 hours, about 1.3 hours, about 1.4 hours, about 1.5 hours, about 1.6 hours, about 1.7 hours, about 1.8 hours, about 1.9 hours, about 2 hours, about 2.2 hours, about 2.4 hours, about 2.5 hours, about 2.6 hours, about 2.8 hours, about 3 hours, about 3.5 hours, about 4 hours, about 4.5 hours, or about 5 hours. NEAR may amplify a target nucleic acid to detectable levels within from about 0.1 hours to about 0.5 hours, from about 0.1 hours to about 1 hour, from about 0.1 hours to about 1.5 hours, from about 0.1 hours to about 2 hours, from about 0.1 hours to about 2.5 hours, from about 0.1 hours to about 3 hours, from about 0.1 hours to about 3.5 hours, from about 0.1 hours to about 4 hours, from about 0.1 hours to about 4.5 hours, from about 0.1 hours to about 5 hours, from about 0.5 hours to about 1 hour, from about 0.5 hours to about 1.5 hours, from about 0.5 hours to about 2 hours, from about 0.5 hours to about 2.5 hours, from about 0.5 hours to about 3 hours, from about 0.5 hours to about 3.5 hours, from about 0.5 hours to about 4 hours, from about 0.5 hours to about 4.5 hours, from about 0.5 hours to about 5 hours, from about 1 hour to about 1.5 hours, from about 1 hour to about 2 hours, from about 1 hour to about 2.5 hours, from about 1 hour to about 3 hours, from about 1 hour to about 3.5 hours, from about 1 hour to about 4 hours, from about 1 hour to about 4.5 hours, from about 1 hour to about 5 hours, from about 1.5 hours to about 2 hours, from about 1.5 hours to about 2.5 hours, from about 1.5 hours to about 3 hours, from about 1.5 hours to about 3.5 hours, from about 1.5 hours to about 4 hours, from about 1.5 hours to about 4.5 hours, from about 1.5 hours to about 5 hours, from about 2 hours to about 2.5 hours, from about 2 hours to about 3 hours, from about 2 hours to about 3.5 hours, from about 2 hours to about 4 hours, from about 2 hours to about 4.5 hours, from about 2 hours to about 5 hours, from about 2.5 hours to about 3 hours, from about 2.5 hours to about 3.5 hours, from about 2.5 hours to about 4 hours, from about 2.5 hours to about 4.5 hours, from about 2.5 hours to about 5 hours, from about 3 hours to about 3.5 hours, from about 3 hours to about 4 hours, from about 3 hours to about 4.5 hours, from about 3 hours to about 5 hours, from about 3.5 hours to about 4 hours, from about 3.5 hours to about 4.5 hours, from about 3.5 hours to about 5 hours, from about 4 hours to about 4.5 hours, from about 4 hours to about 5 hours, or from about 4.5 hours to about 5 hours.

### NEAR Amplification and Detection Reaction Mixtures

NEAR reaction components may comprise a polymerase, a nicking enzyme, dNTPs, and one or more nucleic acid primers. In some embodiments, the reaction may further comprise a reverse transcriptase as described herein. In some embodiments, the polymerase may be an exo-Klenow polymerase. The nicking enzyme may be capable of nicking a single strand of a double stranded nucleic acid sequence. In some embodiments, the nicking enzyme may be capable of nicking an unthiolated strand of a double stranded nucleic acid sequence comprising a thiolated strand and an unthiolated strand. In some embodiments, the nicking enzyme may be capable of nicking a single strand comprising an unthiolated region of a double stranded nucleic acid comprising at one or more thiolated regions and one or more unthiolated regions. In some embodiments, the nicking enzyme is a restriction enzyme capable of nicking a single strand of a double stranded nucleic acid sequence. In some embodiments, the nicking enzyme is a modified restriction enzyme. The nicking enzyme may be a strand-limited restriction enzyme. The restriction enzyme may be HincII. In some embodiments, the restriction enzyme may be AluI, BamHI, EcoP15I, EcoRI, EcoRII, EcoRV, HaeIII, HgaI, HindII, HindIII, HinFI, KpnI, NotI, PstI, PvuII, SacI, SalI, Sau3, ScaI, SmaI, SpeI, SphI, StuI, TaqI, or XbaI, or the like. The nicking enzyme may be Nt.BspQI, Nt.CvPII, Nt.BstNBI, Nb.BsrDI, Nb.BtsI, Nt. AlwI, Nb.BbvCI, Nb.BsmI, Nb.BssSI, Nt. BsmAI, Nb.Bpu10I, Nt. Bpu1101, Nb. Mva1269I, or I-Hmul, or the like. The one or more nucleic acid primers may comprise two primers. For example, the one or more nucleic acid primers may comprise a first primer (e.g., an S1 primer) and a second primer (e.g., an S2 primer). The target nucleic acid may be single stranded DNA or double stranded DNA. In some embodiments, a target nucleic acid comprising RNA may be reverse transcribed into DNA using a reverse transcriptase prior to NEAR amplification. A reverse transcription reaction may comprise primers, dNTPs, and a reverse transcriptase. In some embodiments, the reverse transcription reaction and the NEAR amplification reaction may be performed in the same reaction. A combined RT-NEAR reaction may comprise NEAR primers, reverse transcription primers, dNTPs, a reverse transcriptase, a polymerase, and dNTPs. In some embodiment, the NEAR primers may comprise the reverse transcription primers.

A DETECTR reaction to detect the target nucleic acid sequence may comprise a guide nucleic acid comprising a segment that is reverse complementary to a segment of the target nucleic acid and a programmable nuclease. The programmable nuclease when activated, as described elsewhere herein, exhibits sequence-independent cleavage of a reporter (e.g., a nucleic acid comprising a moiety that becomes detectable upon cleavage of the nucleic acid by the programmable nuclease). The programmable nuclease is activated upon the guide nucleic acid hybridizing to the target nucleic acid. A combined NEAR DETECTR reaction may comprise a polymerase, a restriction enzyme, dNTPs, one or more nucleic acid primers, a guide nucleic acid, a programmable nuclease, and a substrate nucleic acid. A combined RT-NEAR DETECTR reaction may comprise reverse transcription primers, a reverse transcriptase, a polymerase, a restriction enzyme, dNTPs, one or more nucleic acid primers, a guide nucleic acid, a programmable nuclease, and a substrate nucleic acid. In some embodiment, the primers may comprise the reverse transcription primers. NEAR and DETECTR can be carried out in the same sample volume. NEAR and DETECTR can be carried out concurrently in separate sample volumes or in the same sample volume. RT-NEAR and DETECTR can be carried out in the same sample volume. RT-NEAR and DETECTR can be carried out concurrently in separate sample volumes or in the same sample volume. A NEAR reaction may be multiplexed to amplify a plurality of target nucleic acid sequences in a single reaction.

### NEAR Primers and Guide Nucleic Acids

A number of NEAR primers and NEAR primer design methods are consistent with the methods compositions, reagents, enzymes, and kits disclosed herein. NEAR may comprise a set of primers. In some embodiments, NEAR may be an RT-NEAR reaction, a NEAR DETECTR reaction, or an RT-NEAR DETECTR reaction. The set of NEAR primers may comprise two primers, a first primer and a second primer. In some embodiments, a first primer may comprise a sequence of the first region at the 3′ end of the first primer. The sequence of the first region may be from about 16 nucleic acids to about 25 nucleic acids long, or about 20 nucleic acids long. The 3′ end of the first primer may hybridize to the first complementary region of the target. The first complementary region may be 3′ of the target nucleic acid. The first complementary region may be 3′ of a sequence reverse complementary to the target nucleic acid. The first primer may further comprise a cut site 5′ of the sequence of the first region that may be recognized and cleaved by a nicking enzyme. The 3′ end of the first primer may further comprise a recognition site for a nicking enzyme. In some embodiments, the 3′ end of the first primer may further comprise a nicking enzyme stabilization region. The sequence of the second region may be from about 30 nucleic acids to about 38 nucleic acids long. The 3′ end of the second primer may hybridize to the second complementary region. The second complementary region may be 3′ of a sequence reverse complementary to the target nucleic acid. The second complementary region may be 3′ of the target nucleic acid. The second complementary region may be 3′ of a sequence reverse complementary to the target nucleic acid. The second primer may further comprise a cut site 5′ of the sequence of the second region that may be recognized and cleaved by a nicking enzyme. The 3′ end of the second primer may further comprise a recognition site for a nicking enzyme. In some embodiments, the 3′ end of the second primer may further comprise a nicking enzyme stabilization region.

The NEAR primers are designed depending on the site of the optimal guide RNA placement, which may or may not be determined by an available PAM sequence. When performing a NEAR-DETECTR reaction, single-stranded DNA is produced by the designed primers. Because the DETECTR reaction will detection single stranded DNA species, the amplification reaction can be biased to produce more of the particular strand than another. This can be done through changing of the ratio of the forward and reverse primer concentrations. In some embodiments, the concentration of forward primer can be 5 times, 4 times, 3 times, 2 times, or equal to the concentration of reverse primer. In some embodiments, the concentration of reverse primer can be 5 times, 4 times, 3 times, 2 times, or equal to the concentration of forward primer.

In some embodiments, the first region, the second region, or both may be about 8 nucleic acids, about 10 nucleic acids, about 12 nucleic acids, about 14 nucleic acids, about 16 nucleic acids, about 18 nucleic acids, about 20 nucleic acids, about 22 nucleic acids, about 24 nucleic acids, about 26 nucleic acids, about 28 nucleic acids, about 30 nucleic acids, about 32 nucleic acids, about 34 nucleic acids, about 36 nucleic acids, about 38 nucleic acids, about 40 nucleic acids, about 42 nucleic acids, about 44 nucleic acids, about 46 nucleic acids, about 48 nucleic acids, or about 50 nucleic acids long.

In some embodiments, the first region, the second region, or both may be from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, from about 8 to about 50, from about 12 to about 16, from about 12 to about 20, from about 12 to about 24, from about 12 to about 28, from about 12 to about 30, from about 12 to about 32, from about 12 to about 34, from about 12 to about 36, from about 12 to about 38, from about 12 to about 40, from about 12 to about 42, from about 12 to about 44, from about 12 to about 48, from about 12 to about 50, from about 16 to about 20, from about 16 to about 24, from about 16 to about 28, from about 16 to about 30, from about 16 to about 32, from about 16 to about 34, from about 16 to about 36, from about 16 to about 38, from about 16 to about 40, from about 16 to about 42, from about 16 to about 44, from about 16 to about 48, from about 16 to about 50, from about 20 to about 24, from about 20 to about 28, from about 20 to about 30, from about 20 to about 32, from about 20 to about 34, from about 20 to about 36, from about 20 to about 38, from about 20 to about 40, from about 20 to about 42, from about 20 to about 44, from about 20 to about 48, from about 20 to about 50, from about 24 to about 28, from about 24 to about 30, from about 24 to about 32, from about 24 to about 34, from about 24 to about 36, from about 24 to about 38, from about 24 to about 40, from about 24 to about 42, from about 24 to about 44, from about 24 to about 48, from about 24 to about 50, from about 28 to about 30, from about 28 to about 32, from about 28 to about 34, from about 28 to about 36, from about 28 to about 38, from about 28 to about 40, from about 28 to about 42, from about 28 to about 44, from about 28 to about 48, from about 28 to about 50, from about 30 to about 32, from about 30 to about 34, from about 30 to about 36, from about 30 to about 38, from about 30 to about 40, from about 30 to about 42, from about 30 to about 44, from about 30 to about 48, from about 30 to about 50, from about 32 to about 34, from about 32 to about 36, from about 32 to about 38, from about 32 to about 40, from about 32 to about 42, from about 32 to about 44, from about 32 to about 48, from about 32 to about 50, from about 34 to about 36, from about 34 to about 38, from about 34 to about 40, from about 34 to about 42, from about 34 to about 44, from about 34 to about 48, from about 34 to about 50, from about 36 to about 38, from about 36 to about 40, from about 36 to about 42, from about 36 to about 44, from about 36 to about 48, from about 36 to about 50, from about 38 to about 40, from about 38 to about 42, from about 38 to about 44, from about 38 to about 48, from about 38 to about 50, from about 40 to about 42, from about 40 to about 44, from about 40 to about 48, from about 40 to about 50, from about 42 to about 44, from about 42 to about 48, from about 42 to about 50, from about 44 to about 48, from about 44 to about 50, from about 48 to about 50 nucleic acids long.

In some embodiments, the first region, the second region, or both may comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both may comprise a GC content of from about 1% to about 5%, from about 1% to about 10%, from about 1% to about 15%, from about 1% to about 20%, from about 1% to about 25%, from about 1% to about 30%, from about 1% to about 40%, from about 1% to about 50%, from about 1% to about 60%, from about 1% to about 70%, from about 1% to about 80%, from about 1% to about 90%, from about 1% to about 95%, from about 1% to about 99%, from about 5% to about 10%, from about 5% to about 15%, from about 5% to about 20%, from about 5% to about 25%, from about 5% to about 30%, from about 5% to about 40%, from about 5% to about 50%, from about 5% to about 60%, from about 5% to about 70%, from about 5% to about 80%, from about 5% to about 90%, from about 5% to about 95%, from about 5% to about 99%, from about 10% to about 15%, from about 10% to about 20%, from about 10% to about 25%, from about 10% to about 30%, from about 10% to about 40%, from about 10% to about 50%, from about 10% to about 60%, from about 10% to about 70%, from about 10% to about 80%, from about 10% to about 90%, from about 10% to about 95%, from about 10% to about 99%, from about 15% to about 20%, from about 15% to about 25%, from about 15% to about 30%, from about 15% to about 40%, from about 15% to about 50%, from about 15% to about 60%, from about 15% to about 70%, from about 15% to about 80%, from about 15% to about 90%, from about 15% to about 95%, from about 15% to about 99%, from about 20% to about 25%, from about 20% to about 30%, from about 20% to about 40%, from about 20% to about 50%, from about 20% to about 60%, from about 20% to about 70%, from about 20% to about 80%, from about 20% to about 90%, from about 20% to about 95%, from about 20% to about 99%, from about 25% to about 30%, from about 25% to about 40%, from about 25% to about 50%, from about 25% to about 60%, from about 25% to about 70%, from about 25% to about 80%, from about 25% to about 90%, from about 25% to about 95%, from about 25% to about 99%, from about 30% to about 40%, from about 30% to about 50%, from about 30% to about 60%, from about 30% to about 70%, from about 30% to about 80%, from about 30% to about 90%, from about 30% to about 95%, from about 30% to about 99%, from about 40% to about 50%, from about 40% to about 60%, from about 40% to about 70%, from about 40% to about 80%, from about 40% to about 90%, from about 40% to about 95%, from about 40% to about 99%, from about 50% to about 60%, from about 50% to about 70%, from about 50% to about 80%, from about 50% to about 90%, from about 50% to about 95%, from about 50% to about 99%, from about 60% to about 70%, from about 60% to about 80%, from about 60% to about 90%, from about 60% to about 95%, from about 60% to about 99%, from about 70% to about 80%, from about 70% to about 90%, from about 70% to about 95%, from about 70% to about 99%, from about 80% to about 90%, from about 80% to about 95%, from about 80% to about 99%, from about 90% to about 95%, from about 90% to about 99%, or from about 95% to about 99%.

In some embodiments, the first region, the second region, or both may have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both may have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., from about 35° C. to about 85° C., from about 35° C. to about 90° C., from about 35° C. to about 95° C., from about 40° C. to about 45° C., from about 40° C. to about 50° C., from about 40° C. to about 55° C., from about 40° C. to about 60° C., from about 40° C. to about 65° C., from about 40° C. to about 70° C., from about 40° C. to about 75° C., from about 40° C. to about 80° C., from about 40° C. to about 85° C., from about 40° C. to about 90° C., from about 40° C. to about 95° C., from about 45° C. to about 50° C., from about 45° C. to about 55° C., from about 45° C. to about 60° C., from about 45° C. to about 65° C., from about 45° C. to about 70° C., from about 45° C. to about 75° C., from about 45° C. to about 80° C., from about 45° C. to about 85° C., from about 45° C. to about 90° C., from about 45° C. to about 95° C., from about 50° C. to about 55° C., from about 50° C. to about 60° C., from about 50° C. to about 65° C., from about 50° C. to about 70° C., from about 50° C. to about 75° C., from about 50° C. to about 80° C., from about 50° C. to about 85° C., from about 50° C. to about 90° C., from about 50° C. to about 95° C., from about 55° C. to about 60° C., from about 55° C. to about 65° C., from about 55° C. to about 70° C., from about 55° C. to about 75° C., from about 55° C. to about 80° C., from about 55° C. to about 85° C., from about 55° C. to about 90° C., from about 55° C. to about 95° C., from about 60° C. to about 65° C., from about 60° C. to about 70° C., from about 60° C. to about 75° C., from about 60° C. to about 80° C., from about 60° C. to about 85° C., from about 60° C. to about 90° C., from about 60° C. to about 95° C., from about 65° C. to about 70° C., from about 65° C. to about 75° C., from about 65° C. to about 80° C., from about 65° C. to about 85° C., from about 65° C. to about 90° C., from about 65° C. to about 95° C., from about 70° C. to about 75° C., from about 70° C. to about 80° C., from about 70° C. to about 85° C., from about 70° C. to about 90° C., from about 70° C. to about 95° C., from about 75° C. to about 80° C., from about 75° C. to about 85° C., from about 75° C. to about 90° C., from about 75° C. to about 95° C., from about 80° C. to about 85° C., from about 80° C. to about 90° C., from about 80° C. to about 95° C., from about 85° C. to about 90° C., from about 85° C. to about 95° C., or from about 90° C. to about 95° C.

A set of NEAR primers may be designed for use in combination with a DETECTR reaction. The amplified nucleic acid sequence may comprise a sequence that hybridizes to a guide RNA. The amplified nucleic acid sequence may comprise a target nucleic acid. The guide RNA may hybridize to the target nucleic acid. The amplified nucleic acid sequence may comprise corresponding to a guide RNA. The amplified nucleic acid sequence may comprise a sequence reverse complementary to the target nucleic acid. All or part of the guide RNA may be reverse complementary to all or part of the target nucleic acid. The amplified nucleic acid sequence may comprise a protospacer adjacent motif (PAM) positioned next to or near the target sequence. The PAM may be 3′ of the target nucleic acid. In some embodiments, a portion of a sequence that hybridizes the guide RNA may be located between the first region and the second complementary region. The portion of a sequence that hybridizes the guide RNA located between the first region and the second complementary region may comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more of the sequence that hybridizes guide RNA. In some embodiments, the 5′ end of the sequence that hybridizes the guide RNA is 3′ of the 3′ end of the first region and 3′ end of the sequence that hybridizes the guide RNA is 5′ of the 5′ end of the second complementary region. In some embodiments, a portion of a sequence that hybridizes the guide RNA may be located between the second region and the first complementary region. The portion of a sequence that hybridizes the guide RNA located between the second region and the first complementary region may comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more of the sequence that hybridizes the guide RNA. In some embodiments, the 5′ end of the sequence that hybridizes the guide RNA is 3′ of the 3′ end of the second region and 3′ end of the sequence that hybridizes the guide RNA is 5′ of the 5′ end of the first complementary region.

In some embodiments, a sequence that hybridizes the guide RNA may overlap the first region, the first complementary region, the second region, or the second complementary region by no more than no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, or no more than about 95%. In some embodiments, the sequence that hybridizes the guide RNA does not overlap the first region, the first complementary region, the second region, or the second complementary region. In some embodiments, the guide RNA does not hybridize to the first primer or the second primer.

In some embodiments, a NEAR primer set may be designed using a commercially available primer design software. A NEAR primer set may be designed for use in combination with a DETECR reaction, a reverse transcription reaction, or both. One or more methods of designing a set of NEAR primers may be readily apparent to one skilled in the art and may be employed in any of the compositions, kits and methods described herein.

TABLE 5
Exemplary NEAR Primers

| | PRIMER | ALTERNATIVE | |
|---|---|---|---|
| SEQ ID NO: | NAME | NAME | SEQUENCE |
| SEQ ID NO: 35 | M2805 | COV2-M1 | TTGCTTTCGTGGTATT |
| SEQ ID NO: 36 | M2811 | COV2-M2 | GGATGGCTAGTGTAA |
| SEQ ID NO: 37 | R1763 F8 | COV2-F8 | TTTGCCCCCAGCGCTT |
| SEQ ID NO: 38 | R1763 R8 | COV2-R8 | ATGCGCGACATTCCGA |
| SEQ ID NO: 39 | R1763 F7 | COV2-F7 | ATTTGCCCCCAGCGCT |
| SEQ ID NO: 40 | R1763 R7 | COV2-R7 | TGCGCGACATTCCGAA |
| SEQ ID NO: 41 | R1763 F6 | COV2-F6 | AATTTGCCCCCAGCGC |
| SEQ ID NO: 42 | R1763 R6 | COV2-R6 | GCGCGACATTCCGAAG |
| SEQ ID NO: 43 | R1763 F5 | COV2-F5 | CAATTTGCCCCCAGCG |
| SEQ ID NO: 44 | R1763 R5 | COV2-R5 | CGCGACATTCCGAAGA |
| SEQ ID NO: 45 | R1763 F4 | COV2-F4 | ACAATTTGCCCCCAGC |
| SEQ ID NO: 46 | R1763 R4 | COV2-R4 | GCGACATTCCGAAGAA |
| SEQ ID NO: 47 | R1763 F3 | COV2-F3 | CACAATTTGCCCCCAG |
| SEQ ID NO: 48 | R1763 R3 | COV2-R3 | CGACATTCCGAAGAAC |
| SEQ ID NO: 49 | R1763 F2 | COV2-F2 | TCACAATTTGCCCCCA |
| SEQ ID NO: 50 | R1763 R2 | COV2-R2 | GACATTCCGAAGAACG |
| SEQ ID NO: 51 | R1763 F1 | COV2-F1 | TTCACAATTTGCCCCC |
| SEQ ID NO: 52 | R1763 R1 | COV2-R1 | ACATTCCGAAGAACGC |
| SEQ ID NO: 53 | EXAMPLE | EXAMPLE PRIMER | TAGCCAAACAGGAACA |
| | PRIMER 1F | 1F | |
| SEQ ID NO: 54 | EXAMPLE | EXAMPLE PRIMER | GCCAGCAATCTGAGGA |
| | PRIMER 2R | 2R | |

TABLE 6
Exemplary Guides

| | | ALTERNATIVE | |
|---|---|---|---|
| SEQ ID NO: | GUIDE NAME | NAME | Sequence |
| SEQ ID NO: 55 | R1763 | COV2-G1 | TTTGCCCCCAGCGCTTCAGC |
| | | | GTTC |
| SEQ ID NO: 56 | R1765 | COV2-G2 | TTTCGTGGTATTGCTAGTTAC |
| SEQ ID NO: 57 | EXAMPLE | EXAMPLE | CAATCTGAGGAGAGGCCATA |
| | GUIDE 19 | GUIDE 19 | |
| SEQ ID NO: 58 | EXAMPLE | EXAMPLE | AATCTGAGGAGAGGCCATAC |
| | GUIDE 18 | GUIDE 18 | |
| SEQ ID NO: 59 | EXAMPLE | EXAMPLE | ATCTGAGGAGAGGCCATACT |
| | GUIDE 17 | GUIDE 17 | |
| SEQ ID NO: 60 | EXAMPLE | EXAMPLE | TCTGAGGAGAGGCCATACTT |
| | GUIDE16 | GUIDE16 | |
| SEQ ID NO: 61 | EXAMPLE | EXAMPLE | CTGAGGAGAGGCCATACTTG |
| | GUIDE 15 | GUIDE 15 | |
| SEQ ID NO: 62 | EXAMPLE | EXAMPLE | TGAGGAGAGGCCATACTTGT |
| | GUIDE 14 | GUIDE 14 | |
| SEQ ID NO: 63 | EXAMPLE | EXAMPLE | GAGGAGAGGCCATACTTGTT |
| | GUIDE 13 | GUIDE 13 | |
| SEQ ID NO: 64 | EXAMPLE | EXAMPLE | AGGAGAGGCCATACTTGTTC |
| | GUIDE 12 | GUIDE 12 | |
| SEQ ID NO: 65 | EXAMPLE | EXAMPLE | GGAGAGGCCATACTTGTTCC |
| | GUIDE 11 | GUIDE 11 | |
| SEQ ID NO: 66 | EXAMPLE | EXAMPLE | GAGAGGCCATACTTGTTCCT |
| | GUIDE 10 | GUIDE 10 | |
| SEQ ID NO: 67 | EXAMPLE | EXAMPLE | AGAGGCCATACTTGTTCCTG |
| | GUIDE 9 | GUIDE 9 | |
| SEQ ID NO: 68 | EXAMPLE | EXAMPLE | GAGGCCATACTTGTTCCTGT |
| | GUIDE 8 | GUIDE 8 | |
| SEQ ID NO: 69 | EXAMPLE | EXAMPLE | AGGCCATACTTGTTCCTGTT |
| | GUIDE 7 | GUIDE 7 | |
| SEQ ID NO: 70 | EXAMPLE | EXAMPLE | GGCCATACTTGTTCCTGTTT |
| | GUIDE 6 | GUIDE 6 | |
| SEQ ID NO: 71 | EXAMPLE | EXAMPLE | GCCATACTTGTTCCTGTTTG |
| | GUIDE 5 | GUIDE 5 | |
| SEQ ID NO: 72 | EXAMPLE | EXAMPLE | CCATACTTGTTCCTGTTTGG |
| | GUIDE 4 | GUIDE 4 | |
| SEQ ID NO: 73 | EXAMPLE | EXAMPLE | CATACTTGTTCCTGTTTGGC |
| | GUIDE 3 | GUIDE 3 | |
| SEQ ID NO: 74 | EXAMPLE | EXAMPLE | ATACTTGTTCCTGTTTGGCT |
| | GUIDE 2 | GUIDE 2 | |
| SEQ ID NO: 75 | EXAMPLE | EXAMPLE | TACTTGTTCCTGTTTGGCTA |
| | GUIDE 1 | GUIDE 1 | |
| SEQ ID NO: 76 | R1107 | | UAAUUUCUACUAAGUGUAG |
| | | | AUUAGGUCAACAUGCAGCU |
| | | | ACA |
| SEQ ID NO: 77 | R1790 | | GCCACCCCAAAAAUGAAGG |
| | | | GGACUAAAACAUAGGUCAA |
| | | | CAUGCAGCUACA |
| SEQ ID NO: 78 | R1791 | | GCCACCCCAAAAAUGAAGG |
| | | | GGACUAAAACAGUAGGUCA |
| | | | ACAUGCAGCUAC |
| SEQ ID NO: 79 | R1792 | | GCCACCCCAAAAAUGAAGG |
| | | | GGACUAAAACAGGUCAACA |
| | | | UGCAGCUACAGG |
| SEQ ID NO: 80 | R1793 | | GACGAAUGAAGGAAUGCAA |
| | | | CUAGGUCAACAUGCAGCUA |
| | | | CA |
| SEQ ID NO: 81 | R1794 | | GACGAAUGAAGGAAUGCAA |
| | | | CGUAGGUCAACAUGCAGCU |
| | | | AC |
| SEQ ID NO: 82 | R1795 | | GACGAAUGAAGGAAUGCAA |
| | | | CGGUCAACAUGCAGCUACA |
| | | | GG |
| SEQ ID NO: 83 | Cas14a.1 | | Cas14a.1 |
| | tracrRNA-2 | | |

### Amplification and Detection of a Gene of Interest

A DETECTR reaction may be used to detect the presence of a specific target gene in the same. The DETECTR reaction may produce a detectable signal, as described elsewhere herein, in the presence of a target nucleic acid sequence comprising a target gene. The DETECTR reaction may not produce a signal in the absence of the target nucleic acid or in the presence of a nucleic acid sequence that does not comprise the specific SNP allele or comprises a different SNP allele. In some embodiments, a DETECTR reaction may comprise a guide RNA reverse complementary to a portion of a target nucleic acid sequence comprising a specific SNP allele. The guide RNA and the target nucleic acid comprising the specific SNP allele may bind to and activate a programmable nuclease, thereby producing a detectable signal as described elsewhere herein. The guide RNA and a nucleic acid sequence that does not comprise the specific SNP allele may not bind to or activate the programmable nuclease and may not produce a detectable signal. In some embodiments, a target nucleic acid sequence that may or may not comprise a specific SNP allele may be amplified using, for example, a LAMP amplification reaction, an RPA amplification reaction, an SDA amplification reaction, a NEAR amplification reaction, or any other amplification method. In some embodiments, the amplification reaction may be combined with a reverse transcription reaction, a DETECTR reaction, or both. For example, the amplification reaction may be an RT-NEAR reaction, a NEAR DETECTR reaction, or an RT-NEAR DETECTR reaction. In some embodiments, the target nucleic acid sequence can comprise a SNP. In some embodiments, the target nucleic acid sequence can comprise a sequence indicative of a human disease.

A DETECTR reaction, as described elsewhere herein, may produce a detectable signal specifically in the presence of a target nucleic acid sequence comprising a target gene. In addition to the DETECTR reaction, the target nucleic acid having the target gene may be concurrently, sequentially, concurrently together in a sample, or sequentially together in a sample be carried out alongside NEAR or RT-NEAR. For example, the reactions can comprise NEAR and DETECTR reactions, or RT-NEAR and DETECTR reactions. Performing a DETECTR reaction in combination with a NEAR reaction may result in an increased detectable signal as compared to the DETECTR reaction in the absence of the NEAR reaction. In some embodiments, the target nucleic acid sequence can comprise a SNP. In some embodiments, the target nucleic acid sequence can comprise a sequence indicative of a human disease.

In some embodiments, the detectable signal produced in the DETECTR reaction may be higher in the presence of a target nucleic acid comprising target nucleic acid than in the presence of a nucleic acid that does not comprise the target nucleic acid. In some embodiments, the DETECTR reaction may produce a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a target nucleic acid than in the presence of a nucleic acid that does not comprise the target nucleic acid. In some embodiments, the DETECTR reaction may produce a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific SNP allele than in the presence of a nucleic acid that does not comprise the specific SNP allele. In some embodiments, the target nucleic acid sequence can comprise a SNP. In some embodiments, the target nucleic acid sequence can comprise a sequence indicative of a human disease.

A DETECTR reaction may be used to detect the presence of a target nucleic acid associated with a disease or a condition in a nucleic acid sample. The DETECTR reaction may reach signal saturation within about 30 seconds, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 75 minutes, about 80 minutes, or about 85 minutes and be used to detect the presence of a target gene associated with an increased likelihood of developing a disease or a condition in a nucleic acid sample. The DETECTR reaction may be used to detect the presence of a target gene associated with a phenotype in a nucleic acid sample. For example, a DETECTR reaction may be used to detect target gene associated with a disease such as phenylketonuria (PKU), cystic fibrosis, sickle-cell anemia, albinism, Huntington's disease, myotonic dystrophy type 1, hypercholesterolemia, neurofibromatosis, polycystic kidney disease, hemophilia, muscular dystrophy, hypophosphatemic rickets, Rett's syndrome, or spermatogenic failure. A DETECTR reaction may be used to detect a SNP allele associated with an increased risk of cancer, for example bladder cancer, brain cancer, breast cancer, cervical cancer, colon cancer, colorectal cancer, gallbladder cancer, stomach cancer, leukemia, liver cancer, lung cancer, oral cancer, esophageal cancer, ovarian cancer, pancreatic cancer, prostate cancer, skin cancer, testicular cancer, thyroid cancer, neuroblastoma, or lymphoma. A DETECTR reaction may be used to detect a SNP allele associated with an increased risk of a disease, for example Alzheimer's disease, Parkinson's disease, amyloidosis, heterochromatosis, celiac disease, macular degeneration, or hypercholesterolemia. A DETECTR reaction may be used to detect a SNP allele associated with a phenotype, for example, eye color, hair color, height, skin color, race, alcohol flush reaction, caffeine consumption, deep sleep, genetic weight, lactose intolerance, muscle composition, saturated fat and weight, or sleep movement. A DETECTR reaction may also be used to detect the presence of a pathological organism. In some embodiments, the pathological organism is a prokaryote, eukaryote, or a protozoa. In some embodiments, the pathological organism is a virus, an opportunistic pathogen, a parasite, a bacterium, or any combination thereof. In some embodiments, the pathological organism is SARS-CoV-2 or Streptococcus pyogenes.

The terms “sample interface”, “sample input”, “input port”, “input”, “port” as used herein, generally refers to a portion of a device that is configured to receive a sample.

The terms “heating region”, “heated region”, “heat chambers”, “heat volumes”, “heat zones”, “heat surfaces”, “heat areas”, and the like, as used herein, generally refers to a portion of a device that is in thermal communication with a heating unit.

The terms “heater”, “heating unit”, “heating element”, “heat source”, and the like, as used herein, generally refers to an element that is configured to produce heat and is in thermal communication with a portion of a device.

The term “reagent mix”, “reagent master mix”, “reagents”, and the like, as used herein, generally refers to a formulation comprising one or more chemicals that partake in a reaction that the reagent mix is formulated for.

The term “non-cycled temperature profile,” as used herein, generally refers to a temperature profile that is cyclical or sinusoidal in that the temperature profile has an initial temperature, a target temperature, and a final temperature.

The term “capture probe”, “capture molecule”, and the like, as used herein, generally refers to a molecule that selectively binds to a target nucleic acid and only nonspecifically binds to other nucleic acids that can be washed away.

The term “collection tube,” as used herein, generally refers to a compartment that is used to collect a sample and deliver the sample to the sample interface of a device. In some embodiments, the collection tube may be portable. In some embodiments, the collection tube is a syringe.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), or +/−10% of the stated value (or range of values). Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that the throughout the application, data is provided in a number of different formats, and that this data, represents endpoints and starting points, and ranges for any combination of the data points.

As used herein, the terms “thermostable” and “thermostability” refer to the stability of a composition disclosed herein at one or more temperatures, such as an elevated operating temperature for a given reaction. Stability may be assessed by the ability of the composition to perform an activity, e.g., cleaving a target nucleic acid or reporter. Improving thermostability means improving the quantity or quality of the activity at one or more temperatures.

As used herein, the terms “percent identity,” “% identity,” and “% identical” refer to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y in an alignment between the two. Generally, computer programs may be employed for such calculations. Illustrative programs that compare and align pairs of sequences, include ALIGN (Myers and Miller, Comput Appl Biosci. 1988 Mar.; 4 (1): 11-7), FASTA (Pearson and Lipman, Proc Natl Acad Sci USA. 1988 Apr; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95). For the purposes of calculating identity to the sequence, extensions, such as tags, are not included.

As used herein, a “one-pot” reaction refers to a reaction in which more than one reaction occurs in a single volume alongside a programmable nuclease-based detection (e.g., DETECTR) assay. For example, in a one-pot assay, sample preparation, reverse transcription, amplification, in vitro transcription, or any combination thereof, and programmable nuclease-based detection (e.g., DETECTR) assays are carried out in a single volume. In some embodiments, amplification and detection are carried out within a same volume or region of a device (e.g., within a detection region). Readout of the detection (e.g., DETECTR) assay may occur in the single volume or in a second volume. For example, the product of the one-pot DETECTR reaction (e.g., a cleaved detection moiety comprising an enzyme) may be transferred to another volume (e.g., a volume comprising an enzyme substrate) for signal generation and indirect detection of reporter cleavage by a sensor or detector (or by eye in the case of a colorimetric signal).

As used herein, “HotPot” refers to a one-pot reaction in which both amplification (e.g., RT-LAMP) and detection (e.g., DETECTR) reactions occur simultaneously. In many embodiments, a HotPot reaction may utilize a thermostable programmable nuclease which exhibits trans cleavage at elevated temperatures (e.g., greater than 37C).

The terms, “amplification” and “amplifying,” as used herein, refer to a process by which a nucleic acid molecule is enzymatically copied to generate a plurality of nucleic acid molecules containing the same sequence as the original nucleic acid molecule or a distinguishable portion thereof.

The term, “complementary,” as used herein with reference to a nucleic acid refers to the characteristic of a polynucleotide having nucleotides that base pair with their Watson-Crick counterparts (C with G; or A with T/U) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “cleavage assay,” as used herein refers to an assay designed to visualize, quantitate or identify cleavage of a nucleic acid. In some cases, the cleavage activity may be cis-cleavage activity. In some cases, the cleavage activity may be trans-cleavage activity.

Assays which leverage the transcollateral cleavage properties of programmable nuclease enzymes (e.g., CRISPR-Cas enzymes) are often referred to herein as DNA endonuclease targeted CRISPR trans reporter (DETECTR) reactions. As used herein, detection of reporter cleavage (directly or indirectly) to determine the presence of a target nucleic acid sequence may be referred to as “DETECTR”.

The term, “detectable signal,” as used herein refers to a signal that can be detected using optical, fluorescent, chemiluminescent, electrochemical or other detection methods known in the art.

The term, “detecting a nucleic acid” and its grammatical equivalents, as used herein refers to detecting the presence or absence of the target nucleic acid in a sample that potentially contains the nucleic acid being detected.

The term, “effector protein,” as used herein refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. In some embodiments, the complex comprises multiple effector proteins. In some embodiments, the effector protein modifies the target nucleic acid when the complex contacts the target nucleic acid. In some embodiments, the effector protein does not modify the target nucleic acid, but it is fused to a fusion partner protein that modifies the target nucleic acid. A non-limiting example of modifying a target nucleic acid is cleaving (hydrolysis) of a phosphodiester bond. Additional examples of modifying target nucleic acids are described herein and throughout. In some embodiments, the term, “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). The effector protein may be a Cas protein (i.e., an effector protein of a CRISPR-Cas system).

The term, “guide nucleic acid,” as used herein refers to a nucleic acid comprising: a first nucleotide sequence that hybridizes to a target nucleic acid; and a second nucleotide sequence that is capable of being non-covalently bound by an effector protein. The first sequence may be referred to herein as a spacer sequence. The second sequence may be referred to herein as a repeat sequence. In some embodiments, the first sequence is located 5′ of the second nucleotide sequence. In some embodiments, the first sequence is located 3′ of the second nucleotide sequence.

The terms, “non-naturally occurring” and “engineered,” as used herein are used interchangeably and indicate the involvement of human intervention. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by human intervention.

The term, “protospacer adjacent motif (PAM),” as used herein refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The terms, “reporter” and “reporter nucleic acid,” are used interchangeably herein to refer to a non-target nucleic acid molecule that can provide a detectable signal upon cleavage by an effector protein. Examples of detectable signals and detectable moieties that generate detectable signals are provided herein.

The term, “sample,” as used herein generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term, “target nucleic acid,” as used herein refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein when used in reference to a target nucleic acid refers to a sequence of nucleotides that hybridizes to a portion (preferably an equal length portion) of a guide nucleic acid. Hybridization of the guide nucleic acid to the target sequence may bring an effector protein into contact with the target nucleic acid.

The section headings used herein are for organizational purposes only and are not to be construed as limiting the subject matter described.

### ILLUSTRATIVE EMBODIMENTS

The present disclosure provides the following illustrative embodiments.

Embodiment 1. A device for detecting a target nucleic acid, the device comprising:
a sample interface configured to receive a sample;
a first sample chamber in fluid communication with the sample interface and configured to hold a first predetermined volume of the sample;
a first reaction chamber in fluid communication with the first sample chamber and comprising a first set of amplification reagents and a first set of detection reagents, wherein the first set of amplification reagents are configured to amplify a first target nucleic acid, wherein the first set of detection reagents comprises a first programmable nuclease, a first guide nucleic acid configured to bind to the first target nucleic acid, and a first reporter configured to release a first detection moiety when cleaved by the first programmable nuclease following binding of the first guide nucleic acid to the first target nucleic acid;
a first valve disposed between the first sample chamber and the first reaction chamber and configured to regulate flow therebetween;
a first lateral flow assay strip configured to be inserted into the first reaction chamber and capture the first detection moiety when released and thereby detect a presence or absence of the first target nucleic acid in the sample;
a second sample chamber in fluid communication with the sample interface and configured to hold a second predetermined volume of the sample;
a second reaction chamber in fluid communication with the second sample chamber and comprising a second set of amplification reagents and a second set of detection reagents, wherein the second set of amplification reagents are configured to amplify a second target nucleic acid, wherein the second set of detection reagents comprises a second programmable nuclease, a second guide nucleic acid configured to bind to the second target nucleic acid, and a second reporter configured to release a second detection moiety when cleaved by the second programmable nuclease following binding of the second guide nucleic acid to the second target nucleic acid;
a second valve disposed between the second sample chamber and second first reaction chamber and configured to regulate flow therebetween; and
a second lateral flow assay strip configured to be inserted into the second reaction chamber and capture the second detection moiety when released and thereby detect a presence or absence of the second target nucleic acid in the sample, wherein the first and second reaction chambers are fluidly independent of one another.

Embodiment 2. A method for detecting a plurality of target nucleic acids, the method comprising:
applying the sample to the sample interface of the device of Embodiment 1;
transferring the first predetermined volume of the sample into the first sample chamber;
transferring the second predetermined volume of the sample into the second sample chamber;
transferring the first predetermined volume from the first sample chamber to the first reaction chamber;
transferring the second predetermined volume from the second sample chamber to the second reaction chamber;
amplifying the first target nucleic acid in the first reaction chamber;
amplifying the second target nucleic acid in the second reaction chamber;
binding the first target nucleic acid with the first guide nucleic acid, thereby cleaving the first detection moiety from the first reporter in the first reaction chamber;
binding the second target nucleic acid with the second guide nucleic acid, thereby cleaving the second detection moiety from the second reporter in the second reaction chamber;
inserting the first lateral flow assay strip into the first reaction chamber;
inserting the second lateral flow assay strip into the second reaction chamber;
capturing the first detection moiety with the first lateral flow assay strip, thereby detecting the presence of the first target nucleic acid; and
capturing the second detection moiety with the second lateral flow assay strip, thereby detecting the presence of the second target nucleic acid.

Embodiment 3. A device for detecting a target nucleic acid, the device comprising:
a sample interface configured to receive a sample;
a first reaction chamber in fluid communication with the sample interface and comprising a first set of amplification reagents and a first set of detection reagents, wherein the first set of amplification reagents are configured to amplify a first target nucleic acid, wherein the first set of detection reagents comprises a first programmable nuclease, a first guide nucleic acid configured to bind to the first target nucleic acid, and a first reporter configured to release a first detection moiety when cleaved by the first programmable nuclease following binding of the first guide nucleic acid to the first target nucleic acid;
a first lateral flow assay strip in fluid communication with the first reaction chamber and configured to capture the first detection moiety when released and thereby detect a presence or absence of the first target nucleic acid in the sample;
a second reaction chamber in fluid communication with the sample interface and comprising a second set of amplification reagents and a second set of detection reagents, wherein the second set of amplification reagents are configured to amplify a second target nucleic acid, wherein the second set of detection reagents comprises a second programmable nuclease, a second guide nucleic acid configured to bind to the second target nucleic acid, and a second reporter configured to release a second detection moiety when cleaved by the second programmable nuclease following binding of the second guide nucleic acid to the second target nucleic acid; and
a second lateral flow assay strip in fluid communication with the second reaction chamber and configured to capture the second detection moiety when released and thereby detect a presence or absence of the second target nucleic acid in the sample,
wherein the first and second reaction chambers are fluidly independent of one another.

Embodiment 4. A device for detecting a target nucleic acid, comprising:
a sample interface configured to receive a sample;
a reaction chamber in fluid communication with the sample interface;
a programmable nuclease and a guide nucleic acid disposed within the reaction chamber, the guide nucleic acid being configured to bind to a target nucleic acid;
a polymer matrix disposed within the reaction chamber, wherein the polymer matrix comprises a plurality of reporters co-polymerized with at least a first plurality of oligomers,
wherein each of the plurality of reporters is configured to release a detection moiety when cleaved by the programmable nuclease following binding of the guide nucleic acid to the target nucleic acid, and
wherein release of the detection moiety is indicative of a presence or absence of the target nucleic acid.

Embodiment 5. A device for detecting a target nucleic acid, the device comprising:
a sample interface configured to receive a sample;
a first vertical reaction chamber in fluid communication with the sample interface and comprising a first set of amplification reagents and a first set of detection reagents, wherein the first set of amplification reagents are configured to amplify a first target nucleic acid, wherein the first set of detection reagents comprises a first programmable nuclease, a first guide nucleic acid configured to bind to the first target nucleic acid, and a first reporter configured to release a first detection moiety when cleaved by the first programmable nuclease following binding of the first guide nucleic acid to the first target nucleic acid;
a heating reservoir surrounding the first vertical reaction chamber, the heating reservoir configured to heat the first vertical reaction chamber when activated; and
a first lateral flow assay strip disposed at an angle relative to the first vertical reaction chamber and configured to be moved from a first position into a second position in fluid communication with the first reaction chamber, wherein, when in the second position, the first lateral flow assay strip is configured to capture the first detection moiety when released and thereby detect a presence or absence of the first target nucleic acid in the sample.

Embodiment 6. A device for detecting a target nucleic acid, comprising:
a sample interface configured to receive a sample;
a reaction chamber in fluid communication with the sample interface, wherein the reaction chamber comprises a plurality of reporters and thermostable inorganic pyrophosphatase;
a programmable nuclease and a guide nucleic acid disposed within the reaction chamber, the guide nucleic acid being configured to bind to a target nucleic acid;
wherein each of the plurality of reporters is configured to release a detection moiety when cleaved by the programmable nuclease following binding of the guide nucleic acid to the target nucleic acid, and
wherein release of the detection moiety is indicative of a presence or absence of the target nucleic acid.

Embodiment 7. A device for detecting a target nucleic acid, comprising:
a sample interface configured to receive a sample;
a first reaction chamber in fluid communication with the sample interface, wherein the first reaction chamber comprises amplification reagents configured to amplify the sample, said amplification reagents comprising a forward primer, a reverse primer, dNTPs, a DNA polymerase, and a nicking endonuclease;
a second reaction chamber in fluid communication with the first reaction chamber, wherein the second reaction chamber comprises a plurality of reporters;
a programmable nuclease and a guide nucleic acid disposed within the second reaction chamber, the guide nucleic acid being configured to bind to a target nucleic acid;
wherein each of the plurality of reporters is configured to release a detection moiety when cleaved by the programmable nuclease following binding of the guide nucleic acid to the target nucleic acid, and
wherein release of the detection moiety is indicative of a presence or absence of the target nucleic acid in the sample.

Embodiment 8. A device for detecting a target nucleic acid, comprising:
(a) a sample interface configured to receive a sample comprising the target nucleic acid;
(b) a first reaction chamber in fluid communication with the sample interface, wherein the first reaction chamber comprises a plurality of reporters; and
(c) a second reaction chamber in fluid communication with the first reaction chamber, wherein the second reaction chamber comprises an enzyme substrate,
wherein each reporter of the plurality of reporters (i) is immobilized to a surface of the reaction chamber via a tether, (ii) comprised of a programmable nuclease-enzyme fusion protein complexed with a guide nucleic acid, the guide nucleic acid being configured to bind to the target nucleic acid, and (iii) configured to release the programmable nuclease-enzyme fusion protein following cleavage of the tether by the programmable nuclease-enzyme fusion protein bound to the target nucleic acid, and
wherein release of the programmable nuclease-enzyme fusion protein is indicative of a presence or absence of the target nucleic acid in the sample.

Embodiment 9. The device of Embodiment 8, wherein the programmable nuclease-enzyme fusion protein comprises Cas12-HRP.

Embodiment 10. The device of Embodiment 9, wherein the tether of each reporter of the plurality of reporters comprises ssDNA.

Embodiment 11. The device of Embodiment 9, wherein the tether of each reporter of the plurality of reporters comprises RNA.

Embodiment 12. The device of Embodiment 11, wherein the tether of each reporter of the plurality of reporters further comprises DNA.

Embodiment 13. The device of Embodiment 8, wherein the programmable nuclease-enzyme fusion protein comprises Cas12-HRP.

Embodiment 14. The device of Embodiment 13, wherein the tether of each reporter of the plurality of reporters comprises RNA.

Embodiment 15. The device of any one of Embodiments 8-14, wherein the enzyme substrate is HRP substrate.

Embodiment 16. The device of Embodiment 8, wherein the surface is configured to immobilize a nucleic acid.

Embodiment 17. The device of Embodiment 8, wherein the surface comprises streptavidin, biotin, an amine group, a carboxyl group, an epoxy group, an NHS group, a malemide group, or a thiol group.

Embodiment 18. The device of Embodiment 8, wherein the surface is a hydrogel surface.

Embodiment 19. The device of Embodiment 18, wherein the surface comprises an acrydite modification.

Embodiment 20. A device for detecting a target nucleic acid, comprising:
(a) a sample interface configured to receive a sample comprising the target nucleic acid;
(b) a first reaction chamber in fluid communication with the sample interface, wherein the first reaction chamber comprises a plurality of reporters; and
(c) a second reaction chamber in fluid communication with the first reaction chamber, wherein the second reaction chamber comprises an enzyme substrate, and
wherein each reporter of the plurality of reporters comprises a programmable nuclease-split enzyme fusion protein complexed with a guide nucleic acid, the guide nucleic acid being configured to bind to the target nucleic acid, and
wherein release of the programmable nuclease-split enzyme fusion protein is indicative of a presence or absence of the target nucleic acid in the sample.

Embodiment 21. The device of Embodiment 20, wherein the programmable nuclease-split enzyme fusion protein comprises HRP-L.

Embodiment 22. The device of Embodiment 21, further comprising a programmable nuclease-split enzyme fusion protein comprising HRP-S.

Embodiment 23. The device of Embodiment 22, wherein the device is configured to produce a signal upon binding of the programmable nuclease-split enzyme fusion protein comprising HRP-L and binding of the programmable nuclease-split enzyme fusion protein comprising HRP-S to the target nucleic acid.

Embodiment 24. A device for detecting a target nucleic acid, comprising:
(a) a sample interface configured to receive a sample comprising the target nucleic acid;
(b) a first reaction chamber in fluid communication with the sample interface, wherein the first reaction chamber comprises a plurality of reporters, each reporter of the plurality comprising two or more enzymes; and
(c) a surface of the first reaction chamber, wherein each reporter of the plurality of reporters is immobilized to the surface of the first reaction chamber via a tether, and
(d) a second reaction chamber in fluid communication with the first reaction chamber, wherein the second reaction chamber comprises an enzyme substrate, and
wherein cleaving the tether by a programmable nuclease complexed with a guide nucleic acid, the guide nucleic acid being configured to bind to the target nucleic acid, may release the two or more enzymes into solution, and
wherein each enzyme of the two or more enzymes may be contacted with the enzyme substrate in the second reaction chamber to create a detectable and amplified signal indicative of a presence or absence of the target nucleic acid in the sample.

Embodiment 25. The device of Embodiment 24, wherein each enzyme of the two or more enzymes comprises HRP.

Embodiment 26. The device of Embodiment 24, wherein the reporter comprises a poly-HRP comprising a streptavidin comprising the two or more enzymes.

Embodiment 27. The device of Embodiment 24, wherein the tether contacts the surface via a functionality optionally comprising a biotin, an acrydite, or an amine.

Embodiment 28. The device of Embodiment 24, wherein the surface of the first reaction chamber is functionalized with a hydrogel, carboxyl group, or N-Hydroxysuccinimide (NHS).

Embodiment 29. The device of Embodiment 24, wherein the tether comprises a segment of cleavable DNA, cleavable RNA, or a combination thereof.

Embodiment 30. The device of Embodiment 24 or 25, wherein the enzyme substrate comprises 3,3′,5,5′-tetramethylbenzidine (TMB).

Embodiment 31. The device of the Embodiment 24, wherein the surface of the first reaction chamber comprises the inner wall of the first reaction chamber.

Embodiment 32. The device of Embodiment 24, wherein the surface of the first reaction chamber comprises a surface of a bead, wherein the bead is contained within the first reaction chamber.

Embodiment 33. The device of Embodiment 32, wherein the bead is a magnetic bead.

Embodiment 34. A composition comprising a plurality of reporters, wherein (a) each reporter of the plurality of reporters comprises one or more enzymes conjugated to a linker; (b) the linker comprises one or more nucleic acid sections; and (c) the linker (i) comprises a functionality for immobilization to a support, or (ii) is conjugated to the support.

Embodiment 35. The composition of Embodiment 34, wherein the one or more enzymes is a single enzyme.

Embodiment 36. The composition of Embodiment 35, wherein the enzyme is a horseradish peroxidase (HRP).

Embodiment 37. The composition of Embodiment 34, wherein the one or more enzymes comprises a poly-HRP comprising a plurality of HRP enzymes.

Embodiment 38. The composition of Embodiment 37, wherein the plurality of HRP enzymes of the poly-HRP are complexed with streptavidin, and the linker comprises a biotin for conjugation with the poly-HRP.

Embodiment 39. The composition of any one of Embodiments 34-38, wherein the one or more nucleic acid sections comprise one or more sections of DNA, one or more sections of RNA, or a combination thereof.

Embodiment 40. The composition of Embodiment 39, wherein the one or more nucleic acid sections consist of DNA.

Embodiment 41. The composition of Embodiment 39, wherein the one or more nucleic acid sections consist of RNA.

Embodiment 42. The composition of Embodiment 39, wherein the one or more nucleic acid sections comprise one or more sections of DNA and one or more sections of RNA.

Embodiment 43. The composition of any one of Embodiments 39-42, wherein the one or more nucleic acid sections are single-stranded.

Embodiment 44. The composition of any one of Embodiments 39-43, wherein the one or more nucleic acid sections have a combined length of at least 20, 30, 40, or 50 nucleotides.

Embodiment 45. The composition of any one of Embodiments 39-43, wherein at least one of the one or more nucleic acid sections is a substrate for cleavage by a programmable nuclease.

Embodiment 46. The composition of Embodiment 39, wherein the linker comprises (a) a first portion comprising a non-naturally occurring guide nucleic acid, and (b) a second portion comprising a nucleic acid section that is a substrate for cleavage by a programmable nuclease; and further wherein the one or more enzymes are conjugated to the linker via the second portion.

Embodiment 47. The composition of Embodiment 46, wherein the one or more enzymes comprise HRP.

Embodiment 48. The composition of any one of Embodiments 34-47, wherein the linker further comprises one or more hydrocarbon chains.

Embodiment 49. The composition of Embodiment 48, wherein the one or more hydrocarbon chains comprises a hydrocarbon chain between the one or more nucleic acid sections and the support.

Embodiment 50. The composition of Embodiment 48 or 49, wherein the one or more hydrocarbon chains comprises a hydrocarbon chain between the one or more nucleic acid sections and the one or more enzymes.

Embodiment 51. The composition of any one of Embodiments 34-50, wherein the support is a bead, a hydrogel, or the surface of a container.

Embodiment 52. The composition of any one of Embodiments 34-51, wherein the linker comprises the functionality for immobilization to the support.

Embodiment 53. The composition of any one of Embodiments 34-51, wherein the linker is conjugated to the support.

Embodiment 54. The composition of any one of Embodiments 34-53, further comprising a programmable nuclease.

Embodiment 55. A system comprising a reaction chamber, wherein the reaction chamber comprises the composition of any one of Embodiments 34-54.

Embodiment 56. A method for detecting a target nucleic acid in a sample, the method comprising
(a) contacting the sample with the composition of Embodiment 54 to produce a reaction fluid, wherein (i) the linker is conjugated to the support, (ii) the composition further comprises a guide nucleic acid configured to bind to the target nucleic acid, and (iii) the programmable nuclease cleaves the linker in response to formation of a complex comprising the programmable nuclease, the guide nucleic acid, and the target nucleic acid;
(b) separating the reaction fluid from the support;
(c) contacting the separated reaction fluid with a substrate of the one or more enzymes, wherein presence of the one or more enzymes in the fluid produces a detectable signal from the enzyme substrate; and
(d) detecting the detectable signal.

Embodiment 57. A method of detecting a target nucleic acid in a sample, the method comprising:
(a) contacting the sample with the composition of Embodiment 52 to produce a reaction fluid, wherein (i) the composition further comprises a programmable nuclease and a guide nucleic acid, (ii) the guide nucleic acid is configured to bind to the target nucleic acid, and (iii) the programmable nuclease cleaves the linker in response to formation of a complex comprising the programmable nuclease, the guide nucleic acid, and the target nucleic acid;
(b) contacting the reaction fluid with a plurality of beads, wherein each bead reacts with a plurality of the functionalities, thereby immobilizing the functionalities to the beads;
(c) separating the reaction fluid from the beads;
(d) contacting the separated reaction fluid with a substrate of the one or more enzymes, wherein presence of the one or more enzymes in the fluid produces a detectable signal from the enzyme substrate; and
(e) detecting the detectable signal.

Embodiment 58. The method of Embodiment 56 or 57, further comprising concentrating the one or more enzymes prior to contacting the one or more enzymes with the substrate, wherein the concentrating comprises separating the one or more enzymes from at least a portion of the separated reaction fluid.

Embodiment 59. The method of Embodiment 58, wherein concentrating comprises capture of the enzyme on a support.

Embodiment 60. The method of Embodiment 59, wherein contacting the separated reaction fluid with the substrate of the one or more enzymes comprises contacting the enzyme on the support with the substrate 2, 3, 4, 5, or more times.

Embodiment 61. A composition for detecting a target nucleic acid, the composition comprising a programmable nuclease, a guide nucleic acid, a first reporter, an enzyme, and a second reporter, wherein:
(a) the guide nucleic acid is configured to bind to the target nucleic acid;
(b) the programmable nuclease is effective to cleave the first reporter in response to formation of a complex comprising the programmable nuclease, the guide nucleic acid, and the target nucleic acid;
(c) the cleavage of the first reporter is effective to separate a first nucleic acid section from a second nucleic acid section thereof;
(d) the first nucleic acid section is effective to activate the enzyme; and
(e) the activated enzyme is effective to cleave the second reporter to produce a detectable product comprising a detection moiety.

Embodiment 62. The composition of Embodiment 61, wherein the enzyme is an endonuclease and the second reporter comprises a polynucleotide substrate of the enzyme.

Embodiment 63. The composition of Embodiment 62, wherein the endonuclease is a NucC endonuclease.

Embodiment 64. The composition of Embodiment 63, wherein the first nucleic acid section comprises adenosine residues.

Embodiment 65. The composition of Embodiment 64, wherein the adenosine residues comprise cyclic adenylate (cA3).

Embodiment 66. The composition of any one of Embodiments 61-65, wherein the second nucleic acid section comprises RNA residues, optionally wherein the RNA residues comprise a plurality of uracil residues.

Embodiment 67. The composition of any one of Embodiments 61-65, wherein the second nucleic acid section comprises DNA residues, optionally wherein the DNA residues comprise a plurality of thymine residues.

Embodiment 68. The composition of any one of Embodiments 61-67, wherein (a) the second reporter comprises a fluorescent label and a quencher, and (b) cleavage of the second reporter by the activated enzyme is effective to separate the fluorescent label from the quencher.

Embodiment 69. A method of detecting a target nucleic acid in a sample, the method comprising:
(a) contacting the sample with the composition of any one of Embodiments 61-68;
(b) cleaving the first reporter with the programmable nuclease in response to formation of the complex comprising the programmable nuclease, the guide nucleic acid, and the target nucleic acid, thereby releasing the first nucleic acid section;
(c) activating the enzyme with the first nucleic acid section;
(d) cleaving the second reporter with the activated enzyme, thereby producing the detectable product comprising the detection moiety; and
(e) detecting the detection moiety, thereby detecting the presence of the target nucleic acid in the sample.

Embodiment 70. The method of Embodiment 69, wherein (a) the second reporter comprises a polynucleotide substrate of the enzyme, and (b) the enzyme is a NucC.

Embodiment 71. The method of Embodiment 70, wherein step (d) is performed at a temperature of at least 40° C.

Embodiment 72. A composition for detecting a target nucleic acid in a reaction chamber, the composition comprising a programmable nuclease, a guide nucleic acid, a forward primer, a reverse primer, a polymerase, a nicking endonuclease, and a reporter, wherein:
(a) the guide nucleic acid is configured to bind to the target nucleic acid;
(b) the forward primer comprises (i) a 5′ portion comprising a first hairpin, and (ii) a 3′ portion that is configured to bind the target nucleic acid at a first overlapping region with respect to the guide nucleic acid;
(c) the reverse primer comprises (i) a 5′ portion comprising a second hairpin, and (ii) a 3′ portion that is configured to bind a complement of the target nucleic acid at a second overlapping region with respect to the guide nucleic acid;
(d) the first and second hairpins are cleavage substrates for the nicking endonuclease;
(e) the programmable nuclease is effective to cleave the reporter in response to formation of a complex comprising the programmable nuclease, the guide nucleic acid, and (i) the target nucleic acid or (ii) an amplicon of the target nucleic acid; and
(f) the cleavage of the reporter is effective to produce a detectable product comprising a detection moiety.

Embodiment 73. The composition of Embodiment 72, wherein (a) the sequence of the target nucleic acid to which the 3′ portion of the first primer is configured to bind defines a first sequence of the target nucleic acid; (b) the sequence of the 3′ portion of the reverse primer defines a second sequence of the target nucleic acid; and (c) the first sequence and second sequence are separated by about 5 to about 10 nucleotides along the target nucleic acid.

Embodiment 74. The composition of Embodiment 72 or 73, wherein the 3′ portions of the forward primer and reverse primer are about 16 to about 20 nucleotides in length.

Embodiment 75. The composition of any one of Embodiments 72-74, wherein overlap between the 3′ portion of the reverse primer and the sequence to which the guide nucleic acid is configured to bind overlap by 1 to 5 nucleotides, 2 to 5 nucleotides, or 3 nucleotides.

Embodiment 76. The composition of any one of Embodiments 72-75, wherein the first hairpin and/or the second hairpin are 10 to 20 nucleotides in length, 16 to 20 nucleotides in length, or 16 nucleotides in length.

Embodiment 77. The composition of any one of Embodiments 72-76, wherein the programmable nuclease is a Cas protein, optionally wherein the Cas protein is a Cas12 protein or a Cas14 protein.

Embodiment 78. A method of detecting a target nucleic acid in a sample, the method comprising:
(a) contacting the sample with the composition of any one of Embodiments 72-77;
(b) performing nicking enzyme amplification reaction (NEAR) reaction to amplify the target nucleic acid;
(c) forming a complex comprising the programmable nuclease, the guide nucleic acid, and (i) the target nucleic acid, or (ii) an amplicon of the target nucleic acid;
(d) cleaving the reporter with the programmable nuclease activated by formation of the complex, thereby producing the detectable cleavage product; and
(e) detecting the detection moiety, thereby detecting the presence of the target nucleic acid in the sample.

Embodiment 79. The method of Embodiment 78, wherein steps (b) through (d) are performed at about the same temperature.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Electrochemical Detection of the HERC2 Gene Using the DETECTR Reaction

DETECTR has previously been demonstrated to be a powerful technology for detection of pathogens such as SARS-CoV-2 (Broughton et al., 2020). Electrochemical detection has been demonstrated to show a lower limit of detection than fluorescence-based assays by roughly two orders of magnitude (Lou et al., 2015). In this example, an electrochemical probe is incorporated into the DETECTR assay.

The following electrochemical probe: 5′-2XXTTATTXX-3′; Where 2=5′ 6-FAM; X=ferrocene dT and 3=3′ Biotin TEG was used. Additionally, both cyclic voltammetry and square wave voltammetry with the probe, using a DropSens μSTAT. ECL instrument and DropSens screen-printed carbon electrodes were used. A difference in signal between the initial and final timepoints of the DETECTR reaction was observed. A detection of 50 fM. HERC2 target, which is much lower than has been observed with the fluorescence assay was achieved. FIG. 13: presents an oxidation curve for HERC2 DETECTR reaction with electrochemical reporters. Error bars represent standard deviation of two measurements of the same solution, using three traces from each measurement. A 40 nA difference in signal was observed indicating presence of 50 fM of HERC2 target. FIG. 14: presents a reduction curve for HERC2 DETECTR reaction with electrochemical reporters. Error bars represent standard deviation of two measurements of the same solution, using three traces from each measurement. A 30 nA difference in signal indicates the presence of 50 nM of HERC2 after 33 min of reaction. FIG. 15: presents a resultant cyclic voltammogram taken before and after HERC2 DETECTR reaction using 24 μM electrochemical reporter. Each trace is the average of three scans of the same solution and error bars represent standard deviations and the discernable difference between the two voltammograms indicates the presence of 24 μM of reporter.

### Example 2: SARS-CoV-2 Electrochemical DETECTR Reaction

The following electrochemical probe: 5′-2XXTTATTXX-3′; where 2=5′ 6-FAM; X=ferrocene dT; and 3=3′ Biotin TEG was used with a DNA sequence, part of the genome of SARS-CoV-2. Square wave voltammetry measurements were performed using a DropSens μSTAT ECL instrument and DropSens screen-printed carbon electrodes. A difference in signal between the initial and final timepoints of the DETECTR reaction, using 500 fM of target, was detected. FIG. 16 presents an oxidation curve for SARS-CoV-2 DETECTR reaction with electrochemical reporters. Error bars represent standard deviation of three traces from each measurement. The detailed experimental conditions are as follows. A DETECTR mix was prepared according to FIG. 18. Diluted stock reporter 1/10 (1 μL reporter plus 9 μL nuclease-free water) was used to make 240 μM stock. After 30 minute incubation of DETECTR master mix at 37° C. and adding reporter, the reaction mixture was split into two aliquots of 152 μL of master mix plus 8 μL of target (either 1× TE or 10 pM stock concentration gene fragment). The final target concentration was 500 fM. A 55 μL aliquot for measurement was removed at t=0 and then incubated the rest on a heat block at 37° C. for 20 minutes prior to electrochemical measurement. Square wave voltammetry with parameters according to FIG. 19 were used. One 50 μL drop of solution for each measurement was used. Prior to use, each electrode was rinsed with 1 mL of 1× TE and dried with compressed air.

Data Analysis was carried out as follows. The first 2 scans were discarded because they typically have anomalous signals from debris on the electrode that is cleared by voltage application. The remaining 3 scans are averaged for each sample, and the standard deviation and coefficient of variance is calculated. The mean current traces were then imported into PeakFit software for a baseline correction. The corrected traces were then exported and the original CV values were used to calculate standard deviations for the error. It should be noted that the aliquot of reporter used had experienced multiple freeze-thaw cycles. The data from the measurements with the original parameter set were noisy and were not fully analyzed. The location of this peak was different from previous experiments, where a peak was observed around 0.15 V. It is worth noting that the aliquot of reporter had experienced multiple freeze thaw cycles, so it may have degraded. As a control, LAMP buffer to determine the relative signal was also run. The LAMP buffer was already activated with magnesium in a previous experiment, then refrozen). The LAMP buffer was run neat, without any dilution. The signal from the LAMP buffer was much higher than the signal from the DETECTR reactions, as is visible in FIG. 17. It was also found that the signal from the negative control at t=0 was higher than the signal from the other DETECTR reactions. However, this measurement was the second one on that sample and electrode, after a first measurement with the original parameters. In conclusion, SARS-CoV-2 electrochemical DETECTR enabled detectable results for the SARS-CoV-2 virus.

### Example 3: Evaluating Function of Biotin Modified gRNAs for Cas13

The purpose of this experiment was to evaluate biotinylated gRNA functionality with Cas13a both in solution and immobilized on a surface. In this experiment three replicate runs of a biotin-modified gRNA (mod023) and three replicate runs of a non-biotin modified gRNA (R0003) were carried out. Three replicate “no target control,” or NTC runs were carried out for both the mod023 reporter and R0003 control. The procedure was carried out as follows:
(1) gRNAs were diluted to 20 μM.
(2) Cas12 variant (SEQ ID NO: 17) complexing reactions using gRNAs were prepared. The testing and control complexes were diluted to 100 μM final concentrations. Complexing reactions were carried out in two conditions with 3 replicates each resulting in 6 reactions per gRNA. Please see FIG. 16 for complexing mix details.
(3) Sample was incubated for 30 min at 37 C.
(4) Reporter substrate was added.
(5) Reaction was kept on ice until the next step.
(6) 13 μL of 1× MBuffer 1 was added in wells on 384-well plates.
(7) 5 μL of complexing reaction was added.
(8) In a post-amp hood, 2 μL of 1 nM target (respective) was transferred to and kept in a 384 well plate on ice.
(9) Sealed plate with optically clear seal.
(10) Spun down for 30 sec at 2000 rcf.
(11) Read on plate reader with extended gain settings for 30 min at 37 C.

FIGS. 32A and 32B show results, in solution, for mod023, the biotin modified reporter, and R003 the non-biotin-modified reporter, respectively. It was shown that the biotin-modified gRNA had similar performance to the non-biotin-modified gRNA in solution. FIG. 32C shows results for gRNA that was modified with biotin and immobilized to the surface. FIG. 32D shows results for gRNA that was not modified with biotin but was deposited on the surface in the same manner as C. FIG. 32E, similar to FIGS. 32A and 32B, shows results for gRNA that was unmodified and in solution. Together these results showed that with biotin modification and surface immobilization functionality was maintained and DETECTR assay performance was not adversely affected.

### Example 4: Optimizing Reporter Incubation Time on Streptavidin Slides

The objective of this experiment was to verify if a 30 min incubation time was sufficient to produce a strong signal assay signal. Two concentrations were run. The procedure used is as follows:
1. Dilutions of REP072 rep 106 at 1 μM and 5 μM in 1× Wash Buffer were prepared. 1× Wash Buffer is composed of 25 mM Tris, 150 mM NaCl; pH 7.2; 0.1% BSA, and 0.05% Tween®-20 Detergent.
2. The wells of a fresh streptavidin slide were marked as shown in FIG. 33 and FIGS. 34A and 34B using a hydrophobic pen.
3. Three uL of each dilution were spotted on the streptavidin coated slide (in triplicate).
4. The slide was incubated for 30 min at room temperature and covered from light.
5. Three uL of the dilution were removed from each spot.
6. Five uL of 1× wash buffer was added to each spot and mixed up and down 3 times. This wash step was repeated three times.
7. 50 μL of 1× wash buffer was added and incubated for 5 min.
8. The slide was then flicked over a kim wipe to remove all the solution then dabbed gently on all four sides against a kim wipe to clean up the edges.
9. The slides were then imaged on GelDoc (SYBR Blue setting, autoexposure with adjusted gain settings).

FIG. 33 shows results for the test reporter, Rep072, and the negative control, Rep106. The replicates of Rep072 at 5 μM show the strongest signal and the three replicates of Rep072 at 1 μM concentration show the next strongest signal. The negative control reporter, rep 106 shows the same low signal (on none at all) for both 5 μM and 1 μM concentrations. This result shows specific binding of a FAM-bioinylated reporter with a 30 minute incubation time at both 5 μM and 1 μM concentrations. FIGS. 34A-34B show similar results with reporters at 5 mM in FIG. 34A and 2.5 mM in FIG. 34B. The top row of FIGS. 34A and 34B shows spots exhibiting bright fluorescence and the bottom row of FIGS. 34A and 34B show spots exhibiting similarly low fluorescence.

### Example 5: Quencher-Based Reporter Testing for Immobilization

Fluorescent quencher-based reporters were tested in an immobilized DETECTR assay. Streptavidin functionalized plates and biotin labeled reporters were used. FIG. 36 shows sequence and other details for reporters used in this experiment. The following procedure was used:
1. Stocks of the reporters rep072, rep104, rep105, rep117 and rep118 were prepared for binding to the reader plate. Reporter binding details can be seen in FIG. 37A.
2. Complexing reactions were then prepared using the mod018 sequence that is 5′ modified with biotin TEG. See FIG. 36 for more details on sequence mod018. Complexing mix details can be seen in FIG. 37B.
3. Complexing reactions were incubated for 30 min at 37 C.
4. Grid of dilutions of RNP and reporter were prepared with (50:50 ratio) with enough material for 2 reactions each.
5. Wells of a 96-well streptavidin coated plate were pre-rinsed with 100 μL of 1× MBuffer1, twice.
6. 25 μL of complex and 25 μL reporter mix were then added.
7. Sealed plate with foil seal.
8. Binding was then carried out at 25 C for 30 minutes with intermittent shaking (1000 rpm 15 sec every 2 min on Thermomixer).
9. Plates were then spun down briefly.
10. Supernatant was removed.
11. Washed once with 100 μL 1× MBuffer-1. 1× MBuffer-1 is composed of 20 mM Imidazole 7.5, 25 mM KCl, 5 mM MgCl2, 10 μg/mL BSA, 0.01% Igepal Ca-630, and 5% Glycerol.
12. Washed once with 100 μL 1× MBuffer-3. 1× MBuffer-3 is composed of 20 mM HEPES pH 7.5, 2 mM KOAc, 5 mM MgOAc, 1% Glycerol, and 0.00016% Triton-X 100.
13. Added 50 μl of 1× MBuffer3 to each well.
14. Added 5 μL of target/no-target in 1× MBuffer3 a. target volume=5 μL per reaction (GF577 PCR product 1:10).
15. Sealed plate with foil seal.
16. Incubated at 37 C for 90 minutes with intermittent shaking in plate reader measuring FAM intensity.
17. Spun down briefly.
18. Transferred 20 μL of supernatant to wells of 384-well plate and measured FAM fluorescent intensity (single-read).

Results are illustrated in FIGS. 27A-27E. As predicted the positive control shows a positive slope indicating increased binding over the course of the reaction. This is due to the release of FAM dye into solution upon binding and transcleavage as seen in FIG. 27B. In rep104 the cleavage point is between the FAM and the biotin, while the biotin in all reporters is the attachment point to the streptavidin surface. FIG. 27C plots the control, target binding kinetic plot for rep 105. Rep 105 is composed of biotin-FAM-T16-FQ. In this case the streptavidin coated surface emits fluorescence because the region between the FAM dye and the quencher is cleaved upon binding and the quencher is released. FIG. 27D plots the control, target binding kinetic plot for rep 117. Rep117 is composed of biotin-FAM-T20-FQ. In this embodiment, where the reporter is cleaved between the FAM dye and the quencher, thus allowing for release of the quencher in the solution upon binding and transcleavage. This in turn, allows the surface emits fluorescence. FIG. 27E plots the control, target binding kinetic plot for rep118. Rep118 is composed of FAM-T20-biotin-FQ. In this embodiment, the solution emits fluorescence because upon binding the nucleic acid region between the biotin and the FAM is transcleaved, thus releasing the FAM into solution.

### Example 6: Immobilization Optimization-Complex Formation Step

The objective of this experiment was to determine whether binding both the gRNA and reporter to a plate allows the DETECTR assay to be as effective as binding the CAS protein-gRNA complex and reporter. This removes the need to functionalize the surface with the pre-complex of gRNA and CAS protein, allowing for an easier manufacturing process. Additionally, greater specificity can be achieved by allowing for more stringent washes. The following procedure was used.
1. The experiment was designed as shown in FIG. 38A.
2. A stock solution of reporter rep117 was bound to the plate according to the conditions presented in FIG. 38B.
3. Complexing reactions for mod018 (5′ biotin-TEG R1763 SARS-CoV-2 N-gene) and R1763 CDC-N2-Wuhan were then prepared according to the conditions presented in FIG. 39A.
4. Two sets of full complexing mix were made for each and two mixes without Cas12 variant (SEQ ID NO: 17) according to FIG. 39B.
5. Incubated complexing reactions for 30 min at 37C
6. Pre-rinsed wells of 96-well streptavidin coated plate with 50 μL of 1× MBuffer1, twice
7. Added 25 μL reporter to each well.
8. Added 25 μL of complex to A1-D2, 25 μL 1× MB1 to A3-D4, and 25 μL CRNA mix A5-D6.
9. Sealed plate with foil seal.
10. Ran binding reaction at 25 C for 30 minutes with intermittent shaking, 1000 rpm 15 sec every 2 min on Thermomixer
11. Spun streptavidin plate down briefly.
12. Removed supernatant.
13. Washed twice with 100 μL 1× MBuffer-1.
14. Washed once with 100 μL 1× MBuffer-3.
15. Added 50 μl of 1× MBuffer3 to wells A1-D2.
16. Added 25 μL 1×MB3 and 25 μL of complex to “in-solution” wells A3-D4.
17. Added 47.5 μL 1×MB3 and 2.5 μL Cas12 variant (SEQ ID NO: 17) (50 μL MM) to each “prot after” well A5-D6.
18. Added 5 μL of 1:10 diluted purified LAMP product to (+) target wells.
19. Sealed plate with optically clear seal.
20. Read on plate reader-FAM, 37 C, 90 min.

The results of this experiment are shown in FIGS. 29A-29F show that is it possible to add CAS protein with the target and still achieve complexing and signal. FIGS. 29A-29C illustrate results for a first replicate of tests. FIGS. 29D-29F illustrate results for a second replicate of tests. FIGS. 29A and 29D show results where both a biotinylated reporter and a complex of biotinylated RNA and CAS protein were immobilized. Here activity buffer and target were then added. FIGS. 29B and 29E illustrate results where the biotinylated reporter is immobilized and all other reaction components including gRNA and CAS protein are introduced in solution. FIGS. 29C and 29F illustrate results where the biotinylated reporter and biotinylated gRNA are immobilized and then buffer, CAS protein and target are added. In these results it is observed that complexation of CAS protein and gRNA and a reporter signal upon binding can be detected when only gRNA and reporter are immobilized as shown in FIG. 29F.

### Example 7: Demonstration of Immobilized Target Discrimination

The purpose of this experiment was to demonstrate target discrimination for immobilized reporters for the DETECTR reaction. The experiment design used in this experiment is shown in FIG. 40A. The following procedure was used.
1. Experiment planned as shown in FIG. 40A. The experiment included 3 gRNAs including mod018, mod025 and mod024. Two targets and two controls were used. The two targets were N-gene and RNaseP. The two controls were: (1) no target with all other reaction components and (2) water.
2. Stock solution of reporter rep117, later bound to plate, was prepared as shown in FIG. 40B.
3. Complexing reactions were prepared for the three gRNAs: mod018, mod024 and mod025 with reporters:
(1) biotin-TEG R1763 SARS-CoV-2 N-gene,
(2) 5′ biotin-TEG R777 Mammuthus,
(3) 5′ biotin-TEG R1965 RNase P, respectively. The reaction conditions are shown FIG. 41A.
4. Pre-rinsed wells of 96-well streptavidin coated plate with 50 μL of 1× MBuffer1, twice.
5. Added 25 μL reporter to each well.
6. Added 25 μL complexing mix to wells.
7. Sealed plate with foil seal.
8. Ran binding reaction at 25 C for 30 minutes with intermittent shaking (1000 rpm 15 sec every 2 min on Thermomixer).
9. Ran FASTR protocol as follows:
a. Primers used:
   I. SARS-CoV-2: M2062 CDC N2-FWD/M2063 CDC N2-REV.
   II. RNase P: POP7 8F/6R. Please see FIG. 41B for reaction conditions.
   II. a. Pipette 4 μL of master mix into wells of MBS 96-well plate.
b. Added 1 uL twist RNA dilution.
c. 1000 copies/uL: 7.8 μL of 6400c/uL in 42.2 μL H2O.
d. Sealed plate with foil seal at 165 C for 1.5 seconds.
e. Ran the following PCR protocol on the MBS NEXTGENPCR thermocycler according to conditions shown in FIG. 42.
f. Removed plate from thermocycler.
g. Spun down at 2000 rpm for 30 sec.
h. Kept on ice until ready to use.
10. Spun streptavidin plate down briefly.
11. Removed supernatant.
12. Washed twice with 100 μL 1× MBuffer-1.
13. Washed once with 100 μL 1× MBuffer-3.
14. Added 50 μL 1XMB3 15 mM Mg2+.
15. Added 4 μL of target from FASTR to target wells.
16. Sealed plate with optically clear seal.
17. Read on plate reader-FAM, 37 C, 90 min.

Results are shown FIGS. 31A-31C. FIG. 31A presents results for reporter mod018 showing specificity for the N-gene target. FIG. 31B presents results for reporter mod025 showing specificity for the RNaseP target. FIG. 31C presents results for mod024 showing no signal as predicted since no target was present.

### Example 8: Functional Testing for One-Pot RT-LAMP

The purpose of this experiment was to test the functionality of a one-pot reaction composed of both RT-LAMP and Cas12 variant enzyme-based DETECTR master mixes using SEQ ID NO: 17. Both master mixes were co-lyophilized together into one master mix. The reactions are functionally incompatible due to optimal reaction temperature differences, so they were evaluated independently. The RT-LAMP master mix shows reaction characteristics like the liquid controls. Results are shown for the RT-LAMP assay in FIG. 99A and the Cas12 variant assay using SEQ ID NO: 17 in FIG. 99B. The results shown in in FIG. 100 demonstrate a ‘one pot’ reaction, composed of RT-LAMP and Cas12 variant DETECTR master mixes using SEQ ID NO: 17, co-lyophilized into one master mix and reconstituted. The results shown in FIG. 100 demonstrate activity based on the reaction curves.

### Example 9: Cas14a1 DETECTR

The purpose of this experiment was to investigate Cas14a1 which is functional at higher temperatures than RT-LAMP assay requires. In this example the temperatures were run up to 55° C. The results shown in FIG. 101 demonstrate that Cas14a1 was able to be lyophilized and reconstituted with activities comparable to the control.

### Example 10: Functional Testing of a One-Pot Cas12 Variant-Based DETECTR Assay Using SEQ ID NO: 17

The purpose of this experiment was to investigate the performance of pooled and lyophilized master mixes containing both RT-LAMP and DETECTR reaction reagents together. Further this study showed that the pre-lyophilized mixture is stable for two weeks prior to lyophilization. The master mix of RT-LAMP and Cas12 variant-based DETECTR lyophilized together, was functionally tested separately using SEQ ID NO: 17, since the Cas12 variant (SEQ ID NO: 17) is not compatible with the RT-LAMP amplification conditions. These data show robust RT-LAMP and Cas12 variant-based DETECTR activity using SEQ ID NO: 17, comparable to the master mix that was stored at 4° C. for two weeks prior to lyophilization. Results are shown in FIGS. 102A and 102B.

### Example 11: Smaller Volume Lyophilization Reactions

Previous to this example, lyophilized sample volume was demonstrated at 250 μL and performed in glass vials. This example provides data for lyophilization of 25 μL samples in 8-well plastic strip tubes. Additionally, the pre-lyophilization (3 weeks at 4° C.) and values are comparable showing stability of the master mix as seen in FIG. 103. Differential scanning calorimetry results of a lyophilized master mix of reaction reagents, including in 15% Trehalose, are shown in FIG. 104. A half-height mid-point of 109.89° C. was observed. These results taken together demonstrate that the master mix of reaction reagents are stable throughout the lyophilization process at smaller volumes.

### Example 12: One-Pot DETECTR on Handheld Microfluidic Device

In this experiment, the performance of the one-pot DETECTR assay in a handheld microfluidic device was evaluated. Here, one-pot refers to both the RT-LAMP and DETECTR reagents lyophilized as one master mix of reagents. The functions performed on the handheld, microfluidic device included: sample intake; RNA extraction from sample; mixing of the sample with CRISPR reactants; and the transfer of the mixture to a well to heat the mixture to the reaction temperature. In the device, the mixture was heated, and fluorescence was monitored continuously over time. FIG. 106 displays results plotted at 3 different data acquisition settings. The series made of squares shows a setting that did not saturate the detector and therefore displayed the full dynamic range of the signal throughout the life of the assay. From this data it was determined that the DETECTR assay is functional when ran on a miniaturized handheld, microfluidic device.

### Example 13: DETECTR-Based Lateral Flow Assay Strip

The purpose of this example is to demonstrate a DETECTR™-based multiplexed assay using a lateral flow assay (LFA) strip for parallel readout as illustrated in FIG. 107A-107B. To perform the DETECTR™ assay, a surface (10700) is immobilized with programmable nuclease probes (10707) and reporter probes (10701). In this example, the surface is the bottom of a well, separate from the LFA strip. The reporter probes (10701) contain a surface linker (10702), a cleavable nucleic acid sequence (10703), a label (10704) and binding moiety for the flowing capture probe (10705) of the strip. In this example the binding moiety is biotin. The label and binding moiety are attached to the nucleic acid (10703) by a second linker (10706), where in this example the linker is a dendrimer or trebler molecule. The programmable nuclease probe (10707) contains a surface linker and a programmable nuclease (e.g., Cas enzyme) that in turn contains an sgRNA (10708). The sgRNA contains a repeat unit (or hair pin) and a recognition sequence. The recognition sequence is the compliment for a target nucleic acid of the sample. Anti-biotin labeled gold nanoparticles are located in the sample pad (10711) of the LFA strip (10710) as shown in FIG. 107B. In this example, the first step is to contact the surface (10700) with a sample containing target nucleic acids. Upon binding of a target nucleic acid that is complimentary to the sgRNA (10708) of the immobilized programmable nuclease probe (10707), the reporters (10701) immobilized in near proximity, are cleaved by the programmable nuclease, releasing a cleaved section of the reporter (10709) into solution. The sample solution now containing cleaved reporters corresponding to target nucleic acids that were present in the sample, is then contacted to the sample pad (10711) of the lateral flow assay strip (10710). In this example, the sample pad has flowing capture probes (e.g., anti-biotin labeled gold nanoparticles) disposed thereon. Once the sample solution containing released sections of reporters is contacted with the sample pad, said sample solution then flows across the sample pad (for e.g., by being wicked). The cleaved reporters in the sample solution contact and bind to the anti-biotin gold nanoparticle flowing capture probes upon contact in solution. The complex of reporter and nanoparticle is then carried downstream with the rest of the liquid sample by capillary action through the detection region (10712) of the lateral flow assay strip. In this example, the detection region (10712) may comprise six detection spots (10713), where each detection spot (10713) contains a different capture antibody type that is specific for a reporter's dye (10704). In this example, the dye (10704) is FITC and the stationary capture probe is an anti-FITC antibody functionalized to the detection spot (10713) allowing for the specific detection of the FITC labeled reporter among other reporters that are specific for other target nucleic acids. A control line (10714) is present, functionalized with anti-IgG so that all flowing capture probes, not bound to a FITC labeled reporter fragment, (e.g., detection moiety) are captured and detected. It should be noted that in this example, multiple labels and binding moieties were present via the dendritic linker of the detection moiety (10706) to amplify the signal. In this example, multiple detections spots (10713) are present, allowing for the possibility parallel detection of multiplexed samples, as explained in Example 14.

### Example 14: Multiplexed DETECTR-Based Lateral Flow Assay Strip

The purpose of this example is to demonstrate a lateral flow assay strip workflow utilizing a multiplex “Hotpot” assay as illustrated in FIG. 108. In this example, a sample (10801) contains target nucleic acid sequences 1 and 2. The sample (10801) is contacted to the surface (10802) of a well where at each of five locations, D1 through D5, there are different sgRNA's immobilized to the surface. For example, each of the 5 different sgRNA's are part of 5 different programmable nuclease probes (e.g., see FIG. 107) immobilized in the five different locations D1 through D5, as depicted in FIG. 108. Additionally, each of the 5 different sgRNA's are designed to specifically bind to different target nucleic acids in the sample, thus allowing for sample multiplexing. In addition to the immobilized programmable nuclease probes containing sgRNAs, each location, D1 through D5, is functionalized with reporter probes having distinct functional groups. The reporter probes are in close enough proximity to be cleaved by the programmable nuclease probes. Therefore, as described in example 13, reporters are cleaved and released into the solution upon binding between a sgRNA and the target nucleic acid that the sgRNA is designed to bind specifically to. In this example, D4 and D5 each contain reporters labeled with two different labels or capture antibody recognition elements. Once the sample has contacted with the wells (D1 to D5), the respective cleaved sections of reporters are released into the sample solution (as described in Example 13). The sample solution with the released reporters are then contacted with a sample pad, wherein in this example, is situated on lateral flow assay. In this example each detection spot contains a different type of capture antibody, where each capture antibody type specifically binds to a particular label of a reporter. For this example, detection spot (10803) contains the capture antibody anti-FITC, whereas well D5 contains 1) the immobilized Cas-complex including the sgRNA specific to a first target nucleic acid sequence, and 2) the immobilized reporter (10806), which is labeled with FITC. Therefore, upon binding of the first target nucleic acid sequence with the corresponding sgRNA, the linkage between the corresponding immobilized reporter (10806) and corresponding nucleic acid (for example see ref. char. 10701 in FIG. 107) is cleaved, thereby releasing the reporter into solution. By contrast, for this example, detection spot (10804) contains the capture antibody anti-DIG, whereas well D4 contains 1) the immobilized Cas complex including the sgRNA specific to a second target nucleic acid sequence, and 2) the immobilized reporter (10805), which is labeled with DIG. Therefore, upon binding of the second target nucleic acid sequence with the corresponding sgRNA, the linkage between the corresponding immobilized reporter (10805) and corresponding nucleic acid (for example see ref. char. 10701 in FIG. 107) is cleaved, thereby releasing the reporter the solution. The solution now containing cleaved reporters (10805 and 10806) is then contacted to the sample pad of the LFA strip along with chase buffer, where the reporters bind with and pick up flowing capture probes (e.g., anti-biotin-AuNPs) that are disposed on the sample pad. The AuNP-reporter conjugates having reporter (10806) labeled with FITC will selectively bind to detection spot (10803) containing the capture antibody anti-FITC, thus indicating the presence of the first target nucleic acid sequence in the sample. The AuNP-reporter conjugates having reporter (10805) labeled with DIG will selectively bind to detection spot (10804) containing the capture antibody anti-DIG, thus indicating the presence of the second target nucleic acid sequence in the sample. In this manner, parallel detection of 2 or more target nucleic acid sequences present in a multiplexed sample is enabled. In some examples, the detection spots are spaced apart from each other in prescribed locations, such that detection of a reporter at a given detection spot will correlate with a specific target nucleic acid.

### Example 15: HRP-Enhanced DETECTR-Based Lateral Flow Assay Strip

The purpose of this example is to demonstrate horse radish peroxidase (HRP) paper-based detection as illustrated in FIG. 109. Here, “paper-based” refers to a lateral flow assay strip. As in examples 7 and 8, a liquid, blue color sample, containing a target(s) nucleic acid sequence is exposed to a surface upon which CRISPR-Cas/gRNA probes and reporter probes are immobilized. In this example, the reporter probes contain HRP molecules. Upon cleavage of the reporter by the Cas enzyme (e.g., programmable nuclease) following a specific binding event between the target and the guide RNA, the cleaved portion of the reporter is released into the sample solution. In this example, the sample solution having the released sections of reported molecules is then contacted with a lateral flow assay strip comprising a sample pad containing sodium percarbonate, which generates H₂O₂ when hydrated. The rehydration of the sodium percarbonate to form H₂O₂ occurs when the sample is wicked through the sample pad and the lateral flow assay to a detection spot, as described in the previous examples 7 and 8. In this example, the lateral flow assay strip contains the chemical substrates DAB and TMB which activate a color change from blue to red, indicating the presence of HRP, and in turn a “hit” for the target nucleic acid sequence. The chemical catalytic nature of HRP enables signal amplification. Alternatively, the readout can also be accomplished in solution, upon a color change of the sample solution from blue to red.

### Example 16: HRP-Enhanced Multiplexed DETECTR-Based Lateral Flow Assay Strip

The purpose of this example is to demonstrate an HRP-signal enhanced multiplexed lateral flow assay as illustrated in FIG. 110. The immobilized surface (11000) and detection on the lateral flow assay strip (11010) are carried out as described in the previous examples with the exception that signal enhancement is not carried out by gold nanoparticles scattering light. Instead, the anti-biotin labeled AuNP are supplanted by HRP-anti-biotin DAB/TMB. The HRP is activated by sodium percarbonate present in the LFA strip which is rehydrated by the reaction and or chase buffer. In this manner, HRP allows for strong enough signal so as not to require sample amplification such as PCR. Multiplexed detection is accomplished in the same manner as described in Example 14.

### Example 17: Cas13 Based, HRP-Enhanced DETECTR LFA with Capture Oligo Probe Specificity

The purpose of this example is to demonstrate multiplexed target nucleic acid detection utilizing Cas13 RNA cleaving specificity over DNA, HRP-signal enhancement, and capture oligo probe specificity as shown in FIG. 111. In this example, the sample (11100) contains different target nucleic acids. The sample (11100) is then contacted to the surface (11101) of the well that is functionalized at five locations, D1-D5, as described in Example 14. The difference here is that the Cas13 enzyme is present in the Cas-complex probe. Cas13 cleaves RNA but not DNA. This enables the use of a reporter (11102) that contains nucleic acid sequences, one composed of DNA and the other composed of RNA. Upon binding of the target nucleic acid to the sgRNA, the RNA of the reporter is cleaved by the Cas13 enzyme and a fragment containing: a portion of the RNA; the complete DNA sequence; and the FITC label is released into solution. This action is repeated in parallel at each spot D1 through D5 for five different target nucleic acids, producing 5 distinct reporter fragments. The solution is then contacted to the sample pad of the LFA strip, where the sample pad contains HRP-anti-FITC. The FITC labeled reporter fragment then binds to the HRP-anti-FITC, forming a complex (11103) and is carried downstream across the detection region, binding specifically to the detection spot containing a capture oligo that has been designed to be the compliment for the oligo in the complex (11103). In this manner, parallel detection of multiplexed samples as described in Example 14 is possible.

### Example 18: HRP-Enhanced DETECTR™-Based Lateral Flow Assay Strip Utilizing Cas14

The purpose of this example is to demonstrate horse radish peroxidase (HRP) paper-based detection as illustrated in FIG. 109. Here, “paper-based” refers to a lateral flow assay strip. As in examples 13 and 14, a liquid, blue color sample, containing a target(s) nucleic acid sequence is exposed to a surface upon which programmable nuclease probes and reporter probes are immobilized. In this example, the reporter probes contain HRP molecules. Upon cleavage of the reporter by the Cas 14 enzyme following a specific binding event between the target and the guide nucleic acid, the cleaved portion of the reporter is released into the sample solution. In this example, the sample solution is then exposed to a lateral flow assay strip comprising a sample pad containing sodium percarbonate, which generates H₂O₂ when hydrated. The rehydration of the sodium percarbonate to form H₂O₂ occurs when the sample is wicked through the region, as described in the previous examples 13 and 14. Since the substrate contains DAB, TMB, etc . . . the “spot” changes from blue to red, indicating the presence of HRP, and in turn a “hit” for the target nucleic acid sequence. In this manner HRP enables signal amplification. Alternatively, as shown in FIG. 109, the readout can also be accomplished in solution, upon a color change of the sample solution from blue to red.

### Example 19: HRP-T20-Biotin (SEQ ID NO: 1498) DETECTR™-Based Assay

The purpose of this example was to demonstrate an HRP and DETECTR™-based assay. In this example, reporters were cleaved by a Cas complex, or a DNAse enzyme in solution. The cleaved reporter was reacted to HRP-T20-biotin (SEQ ID NO: 1498). The supernatant solution was then added to a reaction volume that contained TMB and H₂O₂ to generate a color signal. The cleaved reporter-HRP conjugate was then detected by optical density measurement of the solution. Optical density measurements were acquired from the beginning of the reaction. The experiment was performed in two sets comprising 2 runs each, where each set was run 1 week apart. DNase and DETECTR™ were used separately in each run of each set. In the DNase runs, 1 nM of HRP target oligo was used in the filled in circle series. Results are shown in FIG. 112. The significance of this example is that multiple turnovers of both HRP and DETECTR™ enable alternative signal amplification to sample amplification such as PCR.

### Example 20: Guide Pooling for Enhanced Target Detection Signal in DETECTR Assays

Guide RNAs that were designed to bind to a different region within a single target nucleic acid were pooled as a strategy for enhancing the target detection signal from DETECTR assays. For examples, in this strategy, each DETECTR™ reaction contained a pool of CRISPR-Cas RNP complexes each of which targeted a different region within a single molecule. As discussed in the paragraphs below, this strategy resulted in increased sensitivity to target detection by using increased number of complexes/single target such that the signal is strong enough to detect within a Poisson distribution (sub-one copy/droplet) and provide a quantitative evaluation of target numbers within a sample.

To test the effect of guide pooling on target detection using the Cas12a nuclease, first, a Cas12a complexing mix was prepared. The R1965 (off-target guide), R1767, R3164, R3178 guides were present in the Cas12a complexing mix in either a pooled-gRNA format (a pool of two or more of the three guides selected from R1767, R3164, or R3178) or in a single-gRNA format (wherein R1767, R3164, R3178 were present individually) and the mix was incubated for 20 minutes at 37° C. A 2-fold dilution series for the template RNA (GF184) was created from a starting dilution concentration (wherein 5.4 μl of GF184 at 0.1 ng/uL was added to 44.6 μl of nuclease-free water). DETECTR master mixes which included the Cas12 complex, Reporter substrate, Fluorescein, Buffer, and diluted template (GF184 or off-target template GF577) were then assembled as shown in Table 7. The DETECTR mixes were then loaded into a Stilla Sapphire chip and placed into the Naica Geode. Crystals were created from thousands of droplets from each sample. No amplification step was performed. The signal from the Sapphire chips was measured in the Red channel. The results of the DETECTR assay showed enhanced Cas12a-based detection of the GF184 target using a pooled-guide format compared to DETECTR Cas12a-based assay using an individual guide format. For example, the DETECTR assays showed an enhanced signal from chamber 5 containing a pool of two guides R1767 and R3178, compared to the signal from chamber 2 or chamber 4 which contained the R1767 and R3178 in individual guide format, respectively, as shown in FIG. 114. Similarly, the DETECTR assays showed an enhanced signal from chamber 9 containing a pool of three guides (R1767, R3164, and R3178), compared to the signal from chamber 5 which contained a pool of two guides (R1767 and R3178) and compared to the signal from chamber 2, chamber 3, or chamber 4 which contained the R1767, R3164, and R3178 in individual guide format, respectively, as shown in FIG. 114.

Table 7 displays conditions, copies per chamber, number of droplets and copies/droplet per chamber.

| | | Copies/ | # | copies/ |
|---|---|---|---|---|
| Chamber | Condition | Chamber | Droplets | droplet |
| 1 | Off Target | 2.5 × 10⁷ | 29336 | 852 |
| | Guide (1965) | | | |
| 2 | Single R1767 | 2.5 × 10⁷ | 26838 | 931 |
| 3 | Single R3164 | 2.5 × 10⁷ | 29590 | 845 |
| 4 | Single R3178 | 2.5 × 10⁷ | 27769 | 900 |
| 5 | 2x pool | 2.5 × 10⁷ | 27929 | 895 |
| | (R1767, R3178) | | | |
| 6 | 2x pool | 1.25 × 10⁷ | 28787 | 434 |
| | (R1767, R3178) | | | |
| 7 | 2x pool | 6.125 × 10⁶ | 27503 | 223 |
| | (R1767, R3178) | | | |
| 8 | 2x pool | 0 | 28814 | 0 |
| | (R1767, R3178) | | | |
| 9 | 3x Pool | 2.5 × 10⁷ | 27881 | 897 |
| | (R1767, R3164, | | | |
| | R3178) | | | |
| 10 | 3x Pool | 1.25 × 10⁷ | 29523 | 423 |
| | (R1767, R3164, | | | |
| | R3178) | | | |
| 11 | 3x Pool | 6.125 × 10⁶ | 28957 | 211 |
| | (R1767, R3164, | | | |
| | R3178) | | | |
| 12 | 3x Pool | 0 | 29087 | 0 |
| | (R1767, R3164, | | | |
| | R3178) | | | |

Enhanced sensitivity to target detection with guide-pooling was observed in the case of Cas13a nuclease also. In these assays, a Cas13a complexing mix was prepared wherein the R002 (off-target guide), R4517, R4519, R4530 guides were present in either a pooled-gRNA format (a pool of two or more of the three guides R4517, R4519, and R4530) or single-gRNA format (wherein R4517, R4519, and R4530 were present individually) and the mix was incubated for 20 minutes at 37 C. DETECTR master mixes which included the Cas13a complex, FAM-U5 Reporter substrate, Buffer, and diluted template SC2 RNA (or off-target template 5S-87) was then assembled as shown in Table 8. The DETECTR mixes were then loaded into a Stilla Sapphire chip and placed into the Naica® Geode system. Crystals were generated from the droplets from each of the samples and incubated at 37° C. (no amplification step was performed). The signal from the Sapphire chips was measured in the Cy5 channel. The results of the DETECTR assay showed enhanced Cas13a-based detection of the SC2 target RNA using a pooled-guide format compared to a Cas13a-based detection of the SC2 target RNA using a single-guide format. For example, the DETECTR assays showed an enhanced signal from chamber 8 (saturated—not displayed), containing the template at a concentration of 1×10⁶ copies, and a pool of the three guides R4517, R4519, and R4530, compared to the signal from chamber 2, chamber 4, or chamber 6 which contained the template at a concentration of 1×10⁶ copies, and the guides R4517, R4519, and R4530 in individual guide format, respectively, as shown in FIG. 115. Similarly, the DETECTR assays showed an enhanced signal from chamber 9 which contained the template at a concentration of 1×10⁵ copies and a pool of three guides (R1767, R3164, and R3178), compared to the signal from chamber 2, chamber 6, or chamber 4, which contained the template at a concentration of 1×10⁶ copies, and which contained the R1767, R3164, and R3178 in individual guide format, respectively, as shown in FIG. 115.

Table 8 displays conditions, copies per chamber, number of droplets per chamber and copies per droplet per chamber.

| | | Copies/ | # | |
|---|---|---|---|---|
| Chamber | Condition | Chamber | Droplets | copies/droplet |
| 1 | Off Target | 1 × 10⁶ | 19960 | 50 |
| | Guide (R002) | | | |
| 2 | Single R4517 | 1 × 10⁶ | 18102 | 55 |
| 3 | Single R4517 | 0 | 19146 | 0 |
| 4 | Single R4519 | 1 × 10⁶ | 18289 | 55 |
| 5 | Single R4519 | 0 | 23324 | 0 |
| 6 | Single R4530 | 1 × 10⁶ | 25402 | 39 |
| 7 | Single R4530 | 0 | 26285 | 0 |
| 8 | 3 pool | 1 × 10⁶ | saturated | ~40 |
| 9 | 3 pool | 1 × 10⁵ | 23209 | 4.3 |
| 10 | 3 pool | 1 × 10⁴ | 24064 | 0.41 |
| 11 | 3 pool | 0 | 21137 | 0 |
| 12 | 3 pool | 1 × 10⁶ | 24885 | 40 |

Next, the sensitivity of a target detection in Cas13a digital droplet DETECTR assays containing guide RNA in either a pooled-guide format versus a single guide format was assayed. DETECTR reaction master-mixes was prepared for each gRNA (R4637, R4638, R4667, R4676, R4684, R4689, R4691, or R4785 (RNaseP)) and included, in addition to the gRNA, the Cas13a nuclease, and the reporter substrate. After complexing, 2 μL of each RNP was combined in either a pooled-gRNA format (a pool of the seven gRNAs, i.e., R4637, R4638, R4676, R4689, R4691, R4667, and R4684) or remained in the single-gRNA format (wherein R4667, R4684, and R4785 (RNAse P were present individually). The template RNAs (Twist SC2, ATCC SC2, and 5s-87 off-target) were diluted to obtain a series of template concentrations. DETECTR reactions directed to the detection of the template RNAs (Twist SC2, ATCC SC2, and 5s-87 off-target template RNAs) were assembled by combining the Cas13a-gRNA RNPs with the diluted template RNA from the previous step as shown in Table 9. The assembled DETECTR reactions were loaded into chambers on a Stilla Sapphire Chip. The Chips were placed into the Naica® Geode system and crystals were generated using the droplet generation program and imaged to reveal droplets that contain detected targets.

The sensitivity of target detection by the DETECTR assays containing the pooled guides (R4637, R4638, R4667, R4676, R4684, R4689, R4691) was compared with the sensitivity of target detection by the DETECTR assays containing the single guides R4684, R4667, R4785 (RNAseP guide) in individual format. Relative quantification performed by counting the number of these positive droplets showed that the samples containing the pooled guide RNAs generated more crystals containing the amplified products per copy of starting target RNA than the samples containing the guide RNAs in individual format as shown in FIG. 116. For example, the number of positive droplets from chamber 1 is higher than the number of droplets in chamber 2 and 3; and the number of droplets from chamber 5 is higher than the number of droplets in chambers 6 and 7 as shown in FIG. 116. Measurement of the target detection signal intensity from the chips also confirmed that the sensitivity of target detection per copy of starting target RNA by the DETECTR assays containing the pooled guides (R4637, R4638, R4667, R4676, R4684, R4689, R4691) was higher than the sensitivity of target detection by the DETECTR assays containing the single guides R4684, R4667, R4785 (RNAseP guide) in individual format as shown in FIG. 117. For example, signal intensity from chamber 1 (containing the seven-guide pool and the Twist SC2 template RNA is higher than the signal intensity in chamber 2 and 3 (containing the R4684, and the R4667 gRNAs in individual format respectively in the presence of the Twist SC2 RNA); and the signal intensity from chamber 5 (containing the seven-guide pool and the ATCC SC2 template RNA) is higher than the signal intensity in chambers 6 and 7 (containing the R4684, and the R4667 gRNAs in individual format respectively, in the presence of the ATCC SC2 RNA) FIG. 117. Similarly, the signal intensity from chamber 5 (containing the seven-guide pool and the ATCC SC2 template RNA) is higher than the signal intensity in chamber 6 (containing the gRNA R4684 in individual format and the ATCC SC2 RNA), the signal intensity from chamber 8 (containing the control RNaseP gRNA in individual format with the ATCC SC2 template RNA) and the signal intensity from chamber 12 (containing the seven pooled gRNAs with no template RNA) FIG. 117.

Relative quantification of the number of droplets containing amplified target (per copy of starting target RNA) observed in chamber 5 (containing the seven-guide pool and the ATCC SC2 template RNA) is higher than the number of droplets observed in chamber 6 (containing the gRNA R4684 in individual format and the ATCC SC2 RNA), the number of droplets observed in chamber 8 (containing the control RNaseP gRNA in individual format with the ATCC SC2 template RNA) and the number of droplets observed in chamber 12 (containing the seven pooled gRNAs with no template RNA) as shown in FIG. 118. The sensitivity of target detection by the assays containing the pooled guides (R4637, R4638, R4667, R4676, R4684, R4689, R4691) was compared with the sensitivity of target detection by the assays containing the single guides R4684, R4667, R4785 (RNAseP guide) in individual format, when the assays were conducted in a benchtop assay format FIG. 119. Results from the bench top assay showed that the samples containing the pooled guides (R4637, R4638, R4667, R4676, R4684, R4689, R4691) was not higher than the sensitivity of target detection by the in the samples containing the single guides R4684, R4667, or R4785 (RNAseP guide) in individual format as shown in FIG. 119.

Table 9 displays the guide pool and template per chamber.

| Chamber | Guide | Template |
|---|---|---|
| 1 | 7 pool | 5000 copies Twist SC2 |
| 2 | R4684 | 5000 copies Twist SC2 |
| 3 | R4667 | 5000 copies Twist SC2 |
| 4 | R4785(RNaseP) | 5000 copies Twist SC2 |
| 5 | 7 pool | 5000 copies ATCC SC2 |
| 6 | R4684 | 5000 copies ATCC SC2 |
| 7 | R4667 | 5000 copies ATCC SC2 |
| 8 | R4785(RNaseP) | 5000 copies ATCC SC2 |
| 9 | 7 pool | 5000 copies 5s-87 |
| 10 | R4684 | 5000 copies 5s-87 |
| 11 | R4667 | 5000 copies 5s-87 |
| 12 | 7 pool | NTC |

### Example 21: Assay Testing Using Bead Immobilized Reporters Following HotPot Protocol

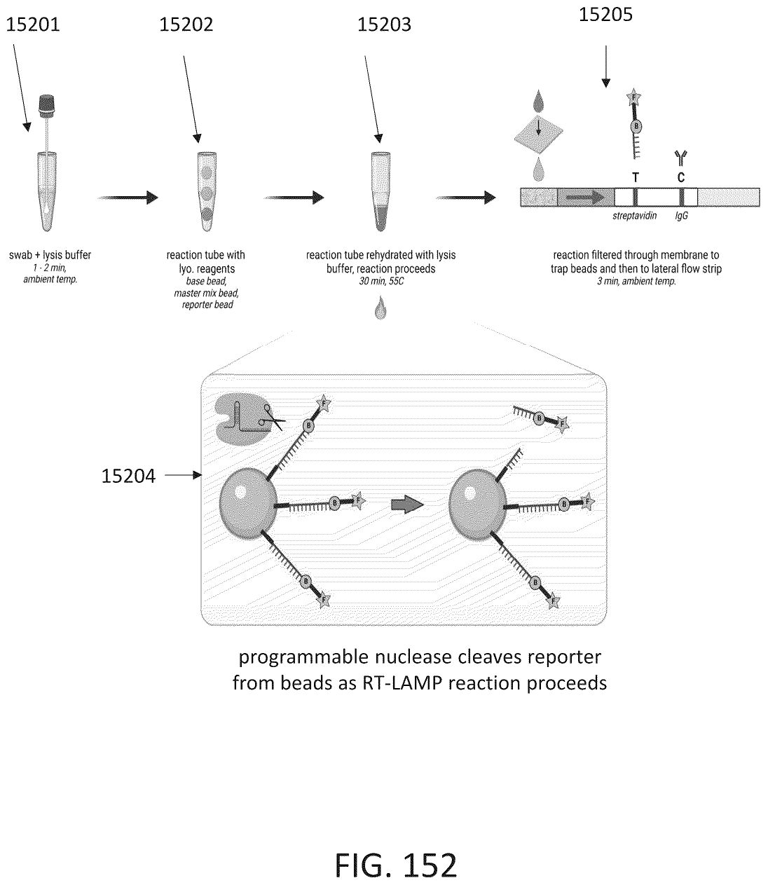

FIG. 152 shows a drawing illustrating the manual HotPot experimental protocol used to test bead-immobilized reporter cleavage. A sample containing target nucleic acids was added to a tube containing lysis buffer (15201). After lysing 1-2 minutes at ambient temperature, the solution was transferred to a reaction tube (15202) containing lyophilized reagents (i.e., the base bead, the master mix bead, and the reporter bead). Contents of the reaction tube were rehydrated and reconstituted with lysis buffer, and the HotPot reaction was started and maintained at 55° C. for 30 minutes (15203). During the reaction, programmable nucleases in the solution cleaved reporter molecules from the beads at the same time as an RT-LAMP reaction proceeded to amplify the target nucleic acids (15204). The reaction medium was then filtered through a membrane to trap the beads and a first portion of the filtered product was used to measure fluorescence thereof on a fluorescence reader. A second portion of the filtered product was applied to a sample pad of a lateral flow strip. The lateral flow strip included a target capture area (T) comprising streptavidin and a control area (C) comprising IgG. The lateral flow strip assay was allowed to run for 3 minutes at ambient temperature (15205) before pictures were taken of the resulting bands at the target capture area T and the control area C.

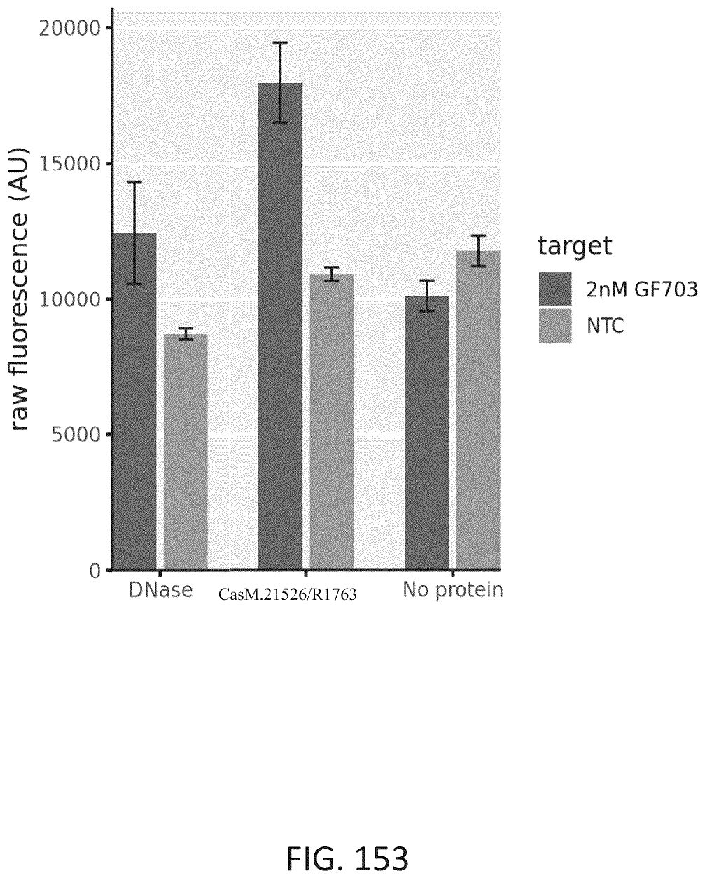

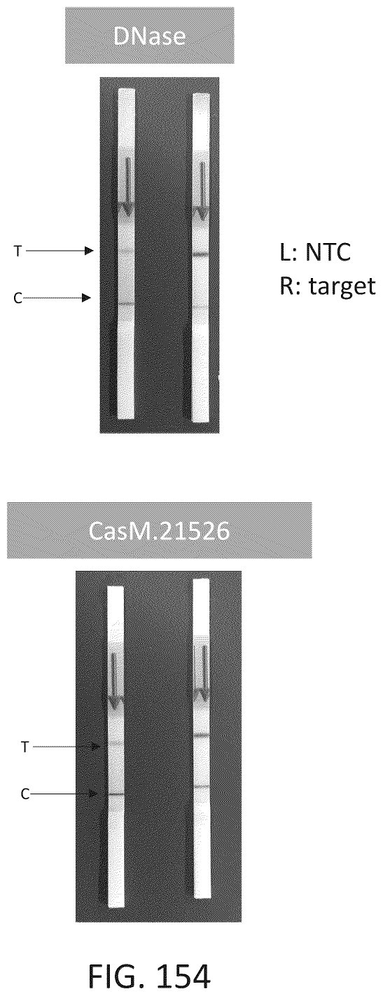

FIG. 153 shows fluorescence DETECTR results with reporters immobilized onto glass beads. Experiments with both DNase and CasM.21526/R1763 showed larger fluorescence signal in the presence of target nucleic acids (2 nM, GF703) compared to the no target control experiments (NTC), thus the HotPot DETECTR reaction successfully cleaved the immobilized reporters from the glass beads. Experiments with CasM.21526 were carried out at 55° C. with H₂.B buffer. Experiments with DNase were carried out at 37° C. with 1× Turbo DNase buffer. FIG. 154 shows photographs of the lateral flow strips to which the DNase and CasM.21526 samples from FIG. 153 were applied.

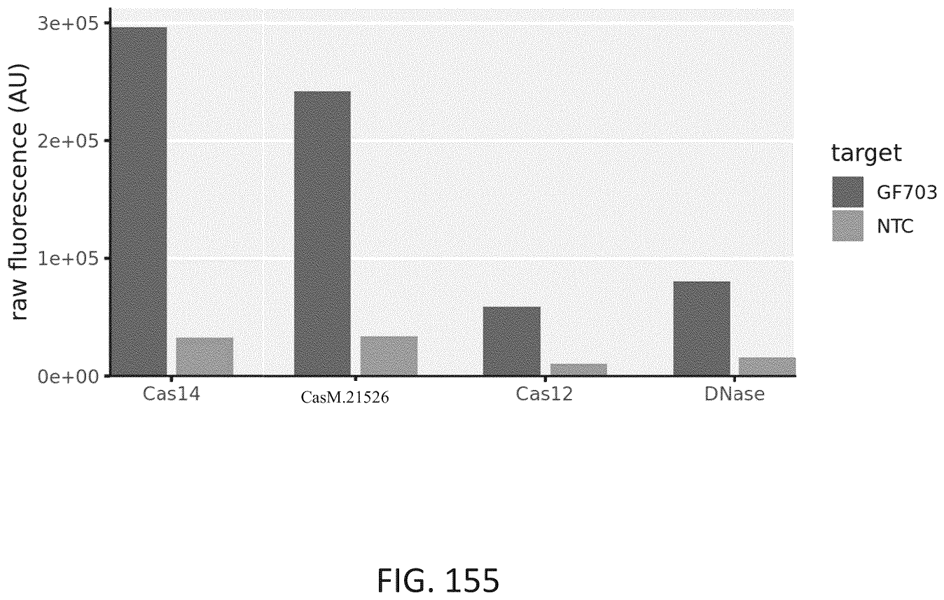

FIG. 155 shows results with maleimide-coated magnetic beads immobilized with thiol-FAM reporter. Experiments with each protein (Cas14, CasM.21526, Cas12) resulted in larger signals with target nucleic acids (GF703) compared to the no target control experiments (NTC), thus the HotPot DETECTR reaction successfully cleaved the immobilized reporters from the maleimide-coated magnetic beads.

### Example 22: HotPot DETECTR-Based Assay for Respiratory Virus Targets

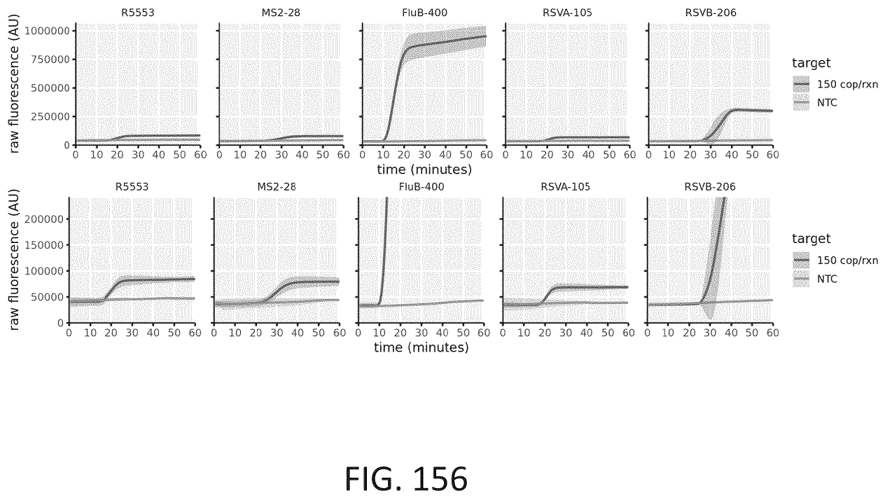

FIG. 156 shows the results HotPot DETECTR-based assays using the protocol shown in FIG. 152 with Cas14a. 1 for multiple different respiratory virus target nucleic acids including SARS-CoV-2, MS2, FluB, RSV-A, and RSV-B. Plots in the top row and the plots in the bottom row show the same data, with different y-axis scale. Each plot shows the raw fluorescence measured during the assay of unique nucleic acid sequences as a function of time. Depending on the target, the saturation signal strength varied from about 70000 AUs to 1000000 AUs, and the saturation time was reached in between about 20 to about 40 minutes (SARS-CoV-2=25 min, MS2=35 min, FluB=20 min, RSV-A=25 min, RSV-B=40 min). Depending on the target, the signal strength from the HotPot DETECTR assay ranged from about 1× to about 25× the signal strength of the SARS-CoV-2 reaction (SARS-CoV-2=1×, MS2=1×, FluB=25×, RSV-A=0.9×, RSV-B=7×).

### Example 23: HotPot Limit of Detection (LOD) Assay for SARS-CoV-2 Target

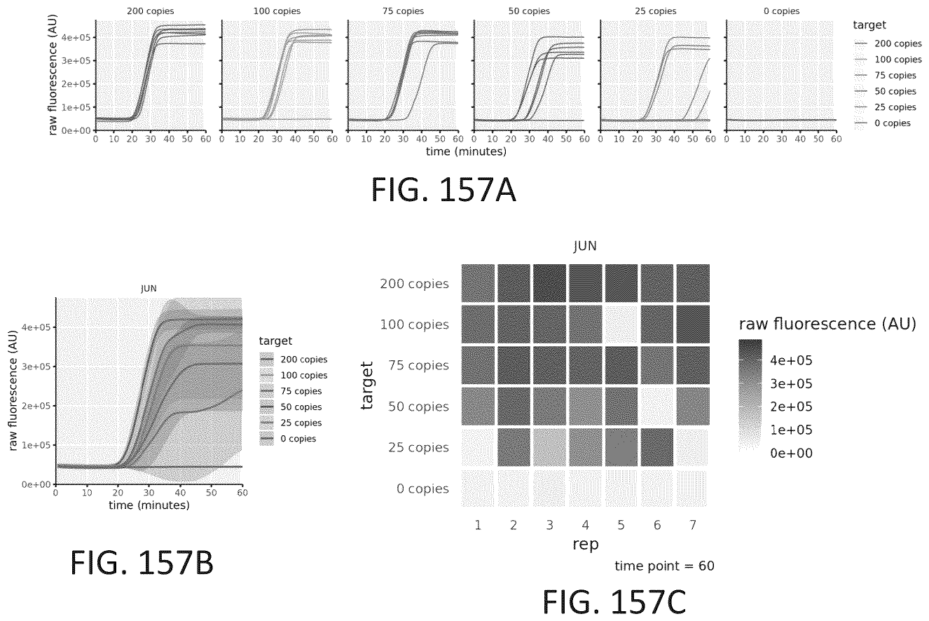

The following describes experiments carried out to determine limit of detection for HotPot reactions using Cas.M21526. The HotPot DETECTR-based assays were run using the protocol shown in FIG. 152. FIG. 157A shows results of experiments carried out using IB15 buffer with different target concentrations ranging from 200 copies to 0 copies. Multiple replicates were conducted at each concentration, and the results of each replicate are shown as individual lines in the plots. FIG. 157B shows an aggregated plot of the data in FIG. 157A where the average and the error from the multiple replicates are plotted in a single plot. FIG. 157C shows the saturation fluorescence heatmap of the multiple replicates (from 1 to 7) of FIG. 157A at different target concentrations. Strong signal was produced at all target concentrations, with about 5 copies being the LOD for the experimental conditions tested.

### Example 24: Additive Screening for SARS-CoV-2 Target HotPot

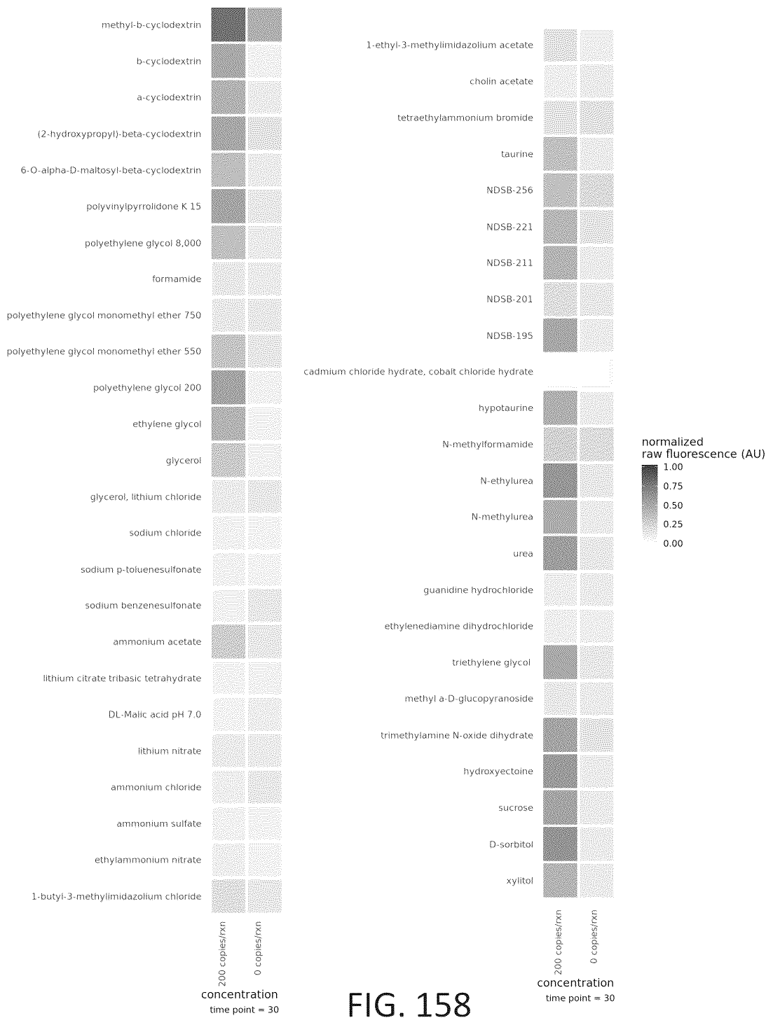

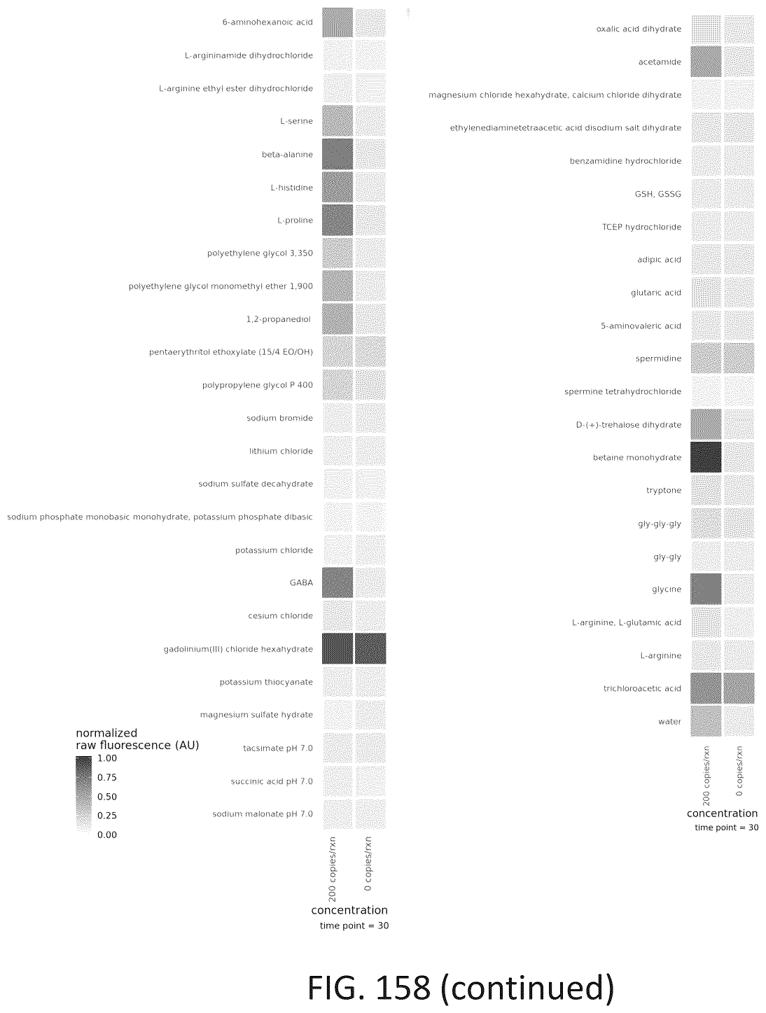

These experiments were conducted to identify potential additives for HotPot assays. 96 potential additives were identified and individually screened for their influence on the output fluorescence. FIG. 158 shows the fluorescence detected from the assays when using a target concentration of 200 copies/reaction (left column) and when using a target concentration of 0 copies/reaction (i.e., no target, right column). The color intensity of the squares in the heatmap indicate the strength of the fluorescence detected.

Additives that were identified to increase the speed of the reaction under the conditions tested included: betaine monohydrate, acetamide, GABA, L-proline, beta-alanine, 6-aminohexanoic acid, urea, methylurea, ethylurea, hypotaurine, NDSB-256, and ammonium acetate.

Additives that were identified to increase the signal strength from the reaction under the conditions tested included: trehalose, xylitol, D-sorbitol, sucrose, and trimethylamine N-oxide dihydrate.

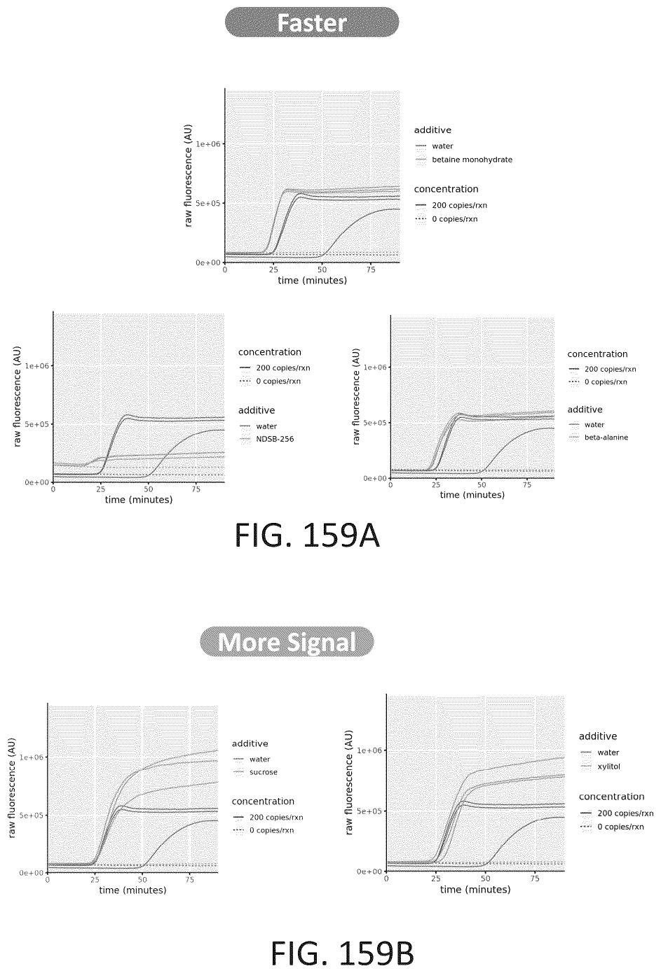

FIG. 159A shows the influence of some additives that increased the speed of the reaction under the conditions tested. The influence of each additive (e.g., betaine monohydrate, NDSB-256, and beta-alanine) is shown against control experiments carried out with added water (i.e., without additive). Experiments carried out with no target (i.e., 0 copies/reaction) showed negligible fluorescence signals. In each scenario, the presence of the additives reduced the amount of time it took for fluorescence signals to reach saturation point (i.e., a plateau in the plot).

FIG. 159B shows the influence of some additives that increased the signal strength of the reaction under the conditions tested. The influence of each additive (e.g., sucrose and xylitol) is shown against control experiments carried out with added water (i.e., without additive). Experiments carried out with no target (i.e., 0 copies/reaction) showed negligible fluorescence signal. In each scenario, the presence of the additives increased the overall fluorescence signal strength generated in the presence of targets.

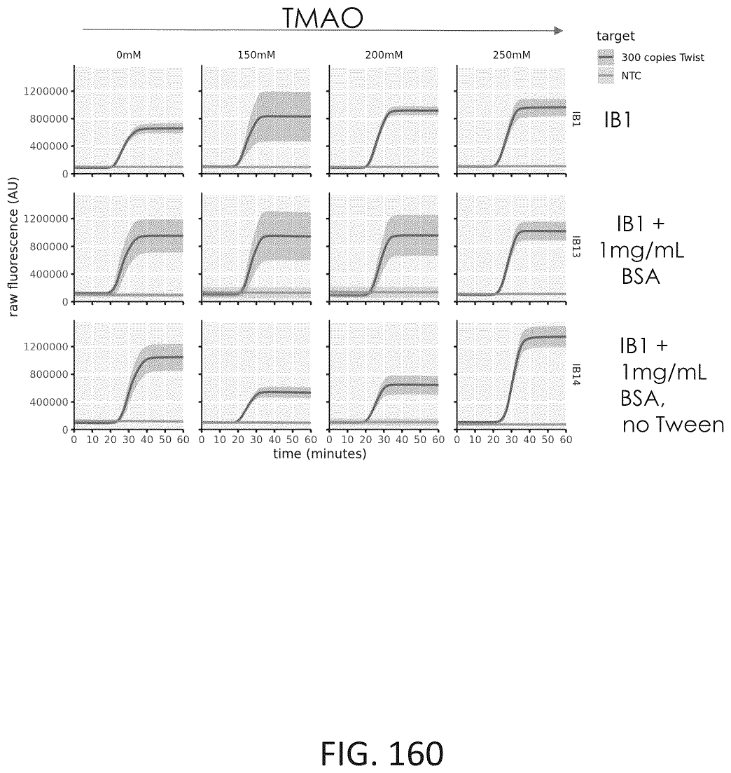

FIG. 160 shows HotPot DETECTR results for various combinations of additives with 300 copies/reaction of Twist SC2 RNA or with no target controls (NTC). Experiments were conducted in the presence of different amounts of trimethylamine N-oxide dihydrate (TMAO, concentrations shown on top of columns), in combination with IB1 buffer (top row), IB13 (comprising IB1 buffer plus 1 mg/ml bovine serum albumin, middle row), or IB14 (comprising IB1 buffer plus 1 mg/ml bovine serum albumin and no Tween, bottom row). All conditions tested exhibited strong DETECTR signals, with 250 mM TMAO appearing to provide the strongest signal in all buffer conditions.

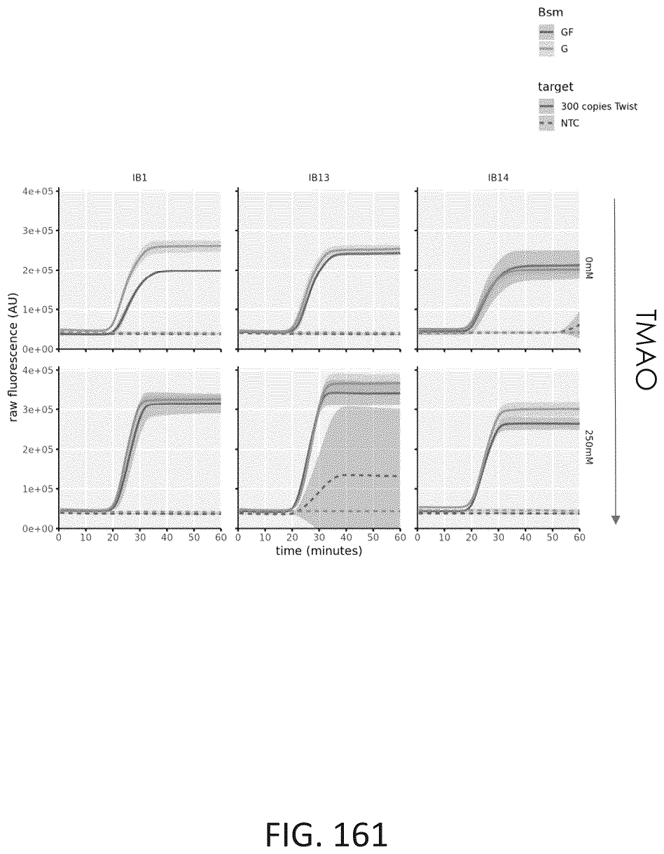

Further HotPot experiments were carried out to determining the effects of using glycerol free (GF) Bsm DNA polymerase and glycerol (G) containing Bsm DNA polymerase with or without 250 mM TMAO added. FIG. 161 shows that both G and GF Bsm DNA polymerases were able to output satisfactory fluorescence signals with each formulation.

### Example 25: DETECTR-Based OnePot and HotPot Reactions Using Reporter Immobilization within Hydrogels

These experiments were carried out to synthesize hydrogels containing immobilized reporters co-polymerized with a mixture of oligomers as described in FIG. 149 and FIGS. 150A-150B and determine their applicability for OnePot and HotPot DETECTR assays. FIG. 149 illustrates the hydrogel structure with a covalently incorporated reporter that was generated via co-polymerization with the reporter.

Reporter was covalently incorporated into PEG hydrogels during polymerization. A 2:1 ratio mixture of unfunctionalized PEG (MW=600 monomers) and PEG-diacrylate (MW=700 monomers) were mixed together with a photoinitiator (2-Hydroxy-2-methylpropiophenone (Darocur 1173)) and 100 μM of Acrydite-modified Reporter 172 (/5Acryd/TTT TTT TTT TTT TTT TTT TT/16-FAMK//3Bio/(SEQ ID NO: 1482)). The mixture was exposed to UV light (365 nm, 200 ms) under a photomask. The mask was configured to polymerize the mix into circular cross-sectional rods of hydrogel 400 μm in diameter. Excess material was washed off hydrogels after polymerization. The acrydite group on the 5′ end of the reporter was covalently reacted with the acrylate groups of PEG-diacrylate oligomers during co-polymerization in order to incorporate the reporter into the hydrogel.

OnePot (using a Cas 12 enzyme) and HotPot (using Cas14a.1) DETECTR reactions were run as described herein by applying the programmable nuclease complexes and target nucleic acids to a tube containing the hydrogels. 6 hydrogels/reaction were added for OnePort DETECTR and 10 hydrogels/reaction for Cas14a. 1 HotPot DETECTR assays. DETECTR reactions were run for 60 min at 37° C. with mixing for Cas 12 OnePot or 60 min at 55° C. with mixing for Cas14a.1 HotPot. Duplicate reactions were run for each of a target RNA and the NTC for both Cas 12 OnePot and Cas14a. 1 HotPot.

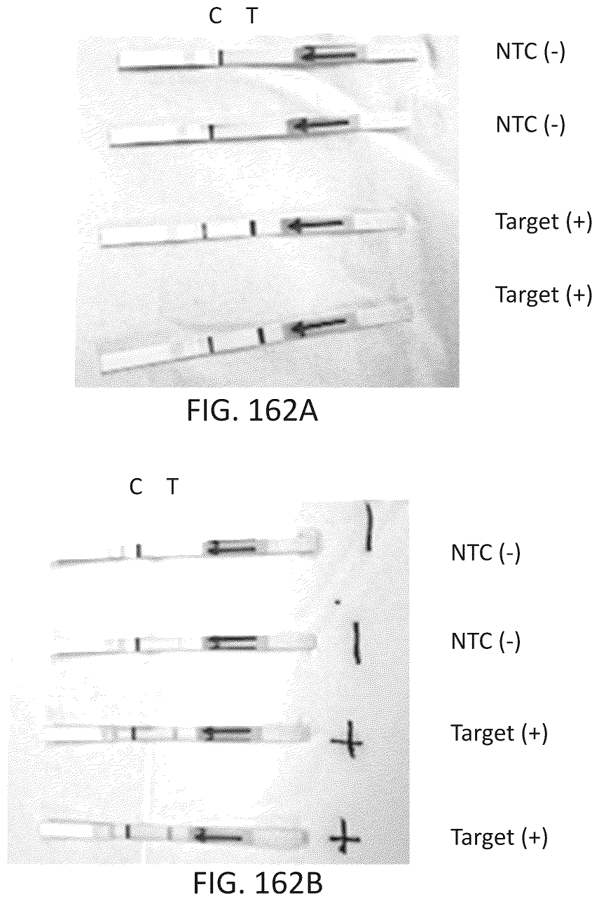

The tubes were then spun down and the supernatant was applied to lateral flow strips. The sample pad of lateral flow strip contained anti-FITC conjugate particles (colloidal gold). If target was present, the supernatant contained cleaved FAM-biotin-labeled reporter molecules which bound to an anti-biotin (e.g., streptavidin) target line on the lateral flow strip. The anti-FITC conjugate particles bound the FAM moiety on the reporter molecules and a target band appeared on lateral flow strips at the anti-biotin target line. If target was not present (as in NTC DETECTR reactions), the supernatant did not contain any FAM-biotin-labeled molecules and nothing bound to the anti-biotin target line. The lateral flow assay strip also contained an anti-IgG flow control line, downstream of the anti-biotin target line, which bound to the anti-FITC moiety of the conjugate particles to confirm that the lateral flow assay functioned properly. FIG. 162A shows the results of the Cas12 OnePot DETECTR assays. FIG. 162B shows the results of the Cas14a. 1 HotPot DETECTR assays. Strong signals were seen in both positive sample replicates while minimal background appeared in NTC replicate strips at the target line.

### Example 26: DETECTR-Based OnePot and HotPot Reactions Using Guide Nucleic Acid and Reporter Immobilization within Hydrogels

This example demonstrates a method of making and using a hydrogel comprising immobilized guide nucleic acids and reporters.

Guide nucleic acids are covalently incorporated into PEG hydrogels during polymerization. A 2:1 ratio mixture of unfunctionalized PEG (MW-600 monomers) and PEG-diacrylate (MW=700 monomers) are mixed together with a photoinitiator (2-Hydroxy-2-methylpropiophenone (Darocur 1173)), 100 μM of Acrydite-modified Reporter 172 (/5Acryd/TTT TTT TTT TTT TTT TTT TT/16-FAMK//3Bio/(SEQ ID NO: 1482)), and 100 μM of Acrydite-modified guide nucleic acids (e.g., R1763 with acrydite modification: /5 Acryd/UAA UUU CUA CUA AGU GUA GAU CCC CCA GCG CUU CAG CGU UC (SEQ ID NO: 1483), or R1965 with acrydite modification:/5Acryd/rUrArAr UrUrUr CrUrAr CrUrAr ArGrUr GrUrAr GrArUr UrUrAr CrArUr GrGrCr UrCrUr GrGrUr CrCrGr Ar G (SEQ ID NO: 1484)). The mixture is exposed to UV light (365 nm, 200 ms) under a photomask. The mask is configured to polymerize the mix into circular cross-sectional rods of hydrogel 400 μm in diameter. Excess material is washed off hydrogels after polymerization. The acrydite group on the 5′ end of the reporters and the acrydite group on the 5′ end of the guide nucleic acids are covalently reacted with the acrylate groups of PEG-diacrylate oligomers during co-polymerization in order to incorporate the reporters and the guide nucleic acids into the hydrogel.

OnePot (using a Cas12 enzyme) and HotPot (using Cas14a.1) DETECTR reactions are run as described herein by applying the programmable nucleases and target nucleic acids to a tube containing the hydrogels. 6 hydrogels/reaction are added for OnePort DETECTR and 10 hydrogels/reaction for Cas14a. 1 HotPot DETECTR assays. DETECTR reactions are run for 60 min at 37° C. with mixing for Cas 12 OnePot or 60 min at 55° C. with mixing for Cas14a. 1 HotPot. Duplicate reactions are run for each of a target RNA and the NTC for both Cas 12 OnePot and Cas14a.1 HotPot.

The tubes are then spun down and the supernatant is applied to lateral flow strips. The sample pad of lateral flow strip contains anti-FITC conjugate particles (colloidal gold). If target is present, the supernatant contains cleaved FAM-biotin-labeled reporter molecules which bind to an anti-biotin (e.g., streptavidin) target line on the lateral flow strip. The anti-FITC conjugate particles bind the FAM moiety on the reporter molecules and a target band appeared on lateral flow strips at the anti-biotin target line. If target is not present, the supernatant does not contain any FAM-biotin-labeled molecules and nothing binds to the anti-biotin target line. The lateral flow assay strip also contains an anti-IgG flow control line, downstream of the anti-biotin target line, which binds to the anti-FITC moiety of the conjugate particles to confirm that the lateral flow assay functions properly.

### Example 27: DETECTR-Based OnePot and HotPot Reactions Using Programmable Nuclease, Guide Nucleic Acid, and Reporter Immobilization within Hydrogels

This example demonstrates a method of making and using a hydrogel comprising immobilized programmable nucleases, guide nucleic acids, and reporter.

Guide nucleic acids are covalently incorporated into PEG hydrogels during polymerization. A 2:1 ratio mixture of unfunctionalized PEG (MW=600 monomers) and PEG-diacrylate (MW=700 monomers) are mixed together with a photoinitiator (2-Hydroxy-2-methylpropiophenone (Darocur 1173)), 100 μM of programmable nuclease (e.g., Cas12 or Cas14a.1), 100 μM of Acrydite-modified Reporter 172 (/5Acryd/TTT TTT TTT TTT TTT TTT TT/16-FAMK//3Bio/(SEQ ID NO: 1482)), and 100 μM of Acrydite-modified guide nucleic acids (e.g., R1763 with acrydite modification:/5Acryd/UAA UUU CUA CUA AGU GUA GAU CCC CCA GCG CUU CAG CGU UC (SEQ ID NO: 1483), or R1965 with acrydite modification:/5Acryd/rUrArAr UrUrUr CrUrAr CrUrAr ArGrUr GrUrAr GrArUr UrUrAr CrArUr GrGrCr UrCrUr GrGrUr CrCrGr Ar G (SEQ ID NO: 1484)). The mixture is exposed to UV light (365 nm, 200 ms) under a photomask. The mask is configured to polymerize the mix into circular cross-sectional rods of hydrogel 400 μm in diameter. Excess material is washed off hydrogels after polymerization. The acrydite group on the 5′ end of the reporters and the acrydite group on the 5′ end of the guide nucleic acids are covalently reacted with the acrylate groups of PEG-diacrylate oligomers during co-polymerization in order to incorporate the reporters and the guide nucleic acids into the hydrogel. The programmable nucleases are immobilized by complexing with guide nucleic acids.

OnePot and HotPot DETECTR reactions are run as described herein by applying target nucleic acids to a tube containing the hydrogels. 6 hydrogels/reaction are added for OnePort DETECTR and 10 hydrogels/reaction for HotPot DETECTR assays. DETECTR reactions are run for 60 min at 37° C. with mixing for OnePot or 60 min at 55° C. with mixing. Duplicate reactions are run for each of a target RNA and the NTC for both OnePot and HotPot.

The tubes are then spun down and the supernatant is applied to lateral flow strips. The sample pad of lateral flow strip contains anti-FITC conjugate particles (colloidal gold). If target is present, the supernatant contains cleaved FAM-biotin-labeled reporter molecules which bind to an anti-biotin (e.g., streptavidin) target line on the lateral flow strip. The anti-FITC conjugate particles bind the FAM moiety on the reporter molecules and a target band appeared on lateral flow strips at the anti-biotin target line. If target is not present, the supernatant does not contain any FAM-biotin-labeled molecules and nothing binds to the anti-biotin target line. The lateral flow assay strip also contains an anti-IgG flow control line, downstream of the anti-biotin target line, which binds to the anti-FITC moiety of the conjugate particles to confirm that the lateral flow assay functions properly.

### Example 28: Positive Feedback Loop System for Amplifying Signals

This example demonstrates a positive feedback loop system for amplifying the signal for each target nucleic acid molecule in a sample as described in FIG. 151.

A mixture comprising programmable nucleases (15101), primary target-specific guide nucleic acids (15102), and primary target nucleic acids (15103) is flowed over a well. The well comprises two types of hydrogels. The first type of hydrogel (15105) contains secondary target-specific guide nucleic acids (15104) that are each immobilized by a single-stranded DNA molecule (15106) onto or into the first hydrogel (15105). The second type of hydrogel (15109) contains double stranded secondary target nucleic acids (15108) immobilized onto or into the second hydrogel (15109). The first programmable nuclease is complexed to the primary target-specific guide nucleic acid (15102). The second programmable nuclease is not bound to a guide nucleic acid.

The first programmable nucleases complexed with the primary target guide nucleic acids (15102) binds to the primary target nucleic acids (15103) to create activated programmable nuclease complexes that trans-cleave nearby single-stranded nucleic acid species. The species include reporters (15111), e.g., free-floating in solution or immobilized to a substrate or hydrogel e.g., adjacent the single-stranded DNA molecules (15106) and/or secondary target nucleic acids, as described herein and the single-stranded DNA molecules (15106) immobilizing the secondary target-specific guide nucleic acids (15104) on or in the first hydrogel (15105). Cleavage of the single-stranded DNA linkers (15104) releases the secondary target-specific guide nucleic acids (15104) from the first hydrogel (15105). This leads to a free-floating population of secondary target-specific guide nucleic acids (15104) in solution. The free-floating secondary target-specific guide nucleic acids (15104) complex with the second programmable nucleases. The second programmable nucleases are then able to bind to the secondary target nucleic acids (15108) immobilized on the second hydrogel (15109) to form activated programmable nuclease complexes that trans-cleave nearby single-stranded nucleic acid species, including reporters (15111) and the single-stranded DNA molecules (15106) on the first hydrogel (15105). This in turn leads to more additional cleavage of reporters (15111) and secondary target-specific guide nucleic acids (15104) in solution, which in turn goes on to create more activated programmable nuclease complexes that release further secondary target-specific guide nucleic acids (15104) into the solution, and so on, thereby amplifying the initial signal from the primary target nucleic acids.

The immobilized nucleic acids (guide nucleic acids, secondary target nucleic acids) comprise fluorescent moieties and quencher moieties. Cleavage of the immobilized nucleic acids separates the quencher moieties from the fluorescent moieties, thereby allowing the fluorescent moieties to produce a detectable fluorescent signal. The fluorescent moieties remain bound to the hydrogels (15105, 15109) after cleavage, which results in an increase in fluorescence at the hydrogel locations. Thus, the amplified signals can be detected by an optical instrument.

### Example 29: Integrated Sodium Acetate LAMP Reaction Consumable Handheld Device

This example demonstrates a handheld device comprising an integrated sodium acetate LAMP reaction consumable handheld device. The purpose of the handheld device is to provide a heating source for programmable nuclease-based detection applications that do not allow for battery or other electrical power supplies. The integrated sodium acetate LAMP reaction consumable provides heating to LAMP and programmable nuclease-based reactions via heat released from the crystallization of liquid-phase sodium acetate. The design of the integrated sodium acetate LAMP reaction consumable allows for uniform heating of the LAMP and programmable nuclease-based reactions by uniformly surrounding the LAMP reaction with sodium acetate heating agent in a heating reservoir.

In the integrated sodium acetate LAMP reaction consumable handheld device, liquid-phase sodium acetate is stored in a heating reservoir as shown in FIG. 163D. Six lateral flow strips (LFS) are placed inside the troughs on the detection tray as shown in FIGS. 163A-163C. LAMP reagents and programmable nuclease-based detection reagents (e.g., programmable nucleases, guide nucleic acids, and reports) are inserted into the 6 reaction chambers shown in FIG. 163A by a user with a pipette. A sample loading layer (e.g., an aliquot chip) is assembled over the reaction chambers as shown in FIG. 163B. In other examples, the reagents come pre-loaded into the reaction chambers and the sample loading layer is pre-assembled onto the rest of the device housing. Samples are added into the sample well as shown in FIGS. 163B-163C. Capillary forces and gravity start the aliquoting of the sample into the sample loading channel and sample loading chambers automatically. A 10 mL syringe (piston drawn up) is threaded on over the sample well as shown in FIG. 163C. The piston of the syringe is then pushed down to drive the remaining sample volume into the sample loading chambers and the appropriate reaction chambers. A frit at the end of each fluid channel downstream of the pressure cracking valve relieves excess pressure as fluids are pushed into the reaction chambers as shown in FIG. 163C. A piercer (not shown), pierces through a heating reservoir film surrounding the heating reservoir to activate the sodium acetate therein. The reaction chamber is heated by the sodium acetate and the isothermal LAMP amplification and programmable nuclease-based detection reactions are initiated. After 40 minutes, the reactions are complete. The detection tray is then pushed forward into the housing to connect the lateral flow assay strips to their respective reaction chambers and the fluid inside the reaction chambers drips down to the lateral flow assay strips and starts the capillary processes as shown in FIG. 163D. Results will be ready 2 minutes after the sample begins the capillary process across the lateral flow assay strips. In the presence of a target nucleic acid(s), the reporters in the reaction chamber(s) will be cleaved during the programmable nuclease-based detection reaction and the cleaved reporter are detected on detection regions or spots of the lateral flow assay strip(s) as described herein.

### Example 30: DETECTR-Based HotPot Reactions Using Thermostable Inorganic Pyrophosphatase

This example demonstrates compositions and methods for HotPot reactions using thermostable inorganic pyrophosphatase (TIPP) to improve signal generation. TIPP is an enzyme that can catalyze the hydrolysis of inorganic pyrophosphates. This example demonstrates compositions and methods for HotPot reactions using thermostable inorganic pyrophosphatase (TIPP) to amplify output signals.

HotPot reactions were carried out under with various compositions as shown in Table 10. In the Experimental Condition, the HotPot reaction was carried out in a solution comprising both TIPP and target nucleic acid. In Control A, the HotPot reaction was carried out with target nucleic acid but without TIPP. In Control B, HotPot reaction was carried out without target RNA. In Control C, HotPot reaction was carried out without TIPP and without target RNA.

| | Amount in (μL) | | | |
|---|---|---|---|---|
| | Experimental | Control | Control | Control |
| Component | Condition | A | B | C |
| Nuclease-free water | 1.975 | 1.975 | 1.975 | 1.975 |
| 5X IB 15 | 2 | 2 | 2 | 2 |
| 10 mM dNTP (NEB) | 1 | 1 | 1 | 1 |
| 100 μM rep033 | 0.1 | 0.1 | 0.1 | 0.1 |
| RNase Inhibitor, Murine | 0.05 | 0.05 | 0.05 | 0.05 |
| (NEB) | | | | |
| Bsm (Thermo) | 0.5 | 0.5 | 0.5 | 0.5 |
| Warmstart RTx (NEB) | 0.125 | 0.125 | 0.125 | 0.125 |
| Cas14a complexing | 1 | 1 | 1 | 1 |
| reaction | | | | |
| 10X Primer Mix | 1 | 1 | 1 | 1 |
| TIPP (2U/μL) | 0.25 | — | 0.25 | — |
| Target RNA | 2 | 2 | — | — |
| Total | 10 | 9.75 | 8 | 7.75 |

Briefly, in a first experimental example, Cas14a effector proteins were complexed with sgRNA for 30 minutes at 37° C. The 1× concentration of proteins was 40 nM and the final concentration of sgRNA was 40 nM. 1 uL of the Cas14a complexing reaction was combined with the HotPot components listed in Table 9 for each experimental or control condition listed. Reactions were carried out at 55° C. for 60 minutes. Trans cleavage activity was detected by fluorescence signal upon cleavage of a fluorophore-quencher reporter in the HotPot DETECTR reaction. FIG. 164 shows HotPot reaction results under the various conditions. The experimental condition, compared to control A without TIPP, had greater than 5-fold increase in the output fluorescence signal, while control A still had significantly higher signal than control D (no TIPP or target nucleic acid) and both conditions were able to detect the target nucleic acid. Control B without target RNA had about the same output fluorescence signal as control C without TIPP and without target RNA.

In a second experimental example, a 2:1 ratio mixture of unfunctionalized PEG (MW=600 monomers) and PEG-diacrylate (MW=700 monomers) were mixed together with a photoinitiator (2-Hydroxy-2-methylpropiophenone (Darocur 1173)) and 100 μM of Acrydite-modified “Rep172” reporter (/5Acryd/TTT TTT TTT TTT TTT TTT TT/16-FAMK//3Bio/(SEQ ID NO: 1482)). The mixture was exposed to UV light (365 nm, 200 ms) under a photomask to generate circular cross-section rods of hydrogel containing immobilized reporters. Cas14a effector proteins were complexed with sgRNA for 30 minutes at 37° C. The 1× concentration of proteins was 40 nM and the final concentration of sgRNA was 40 nM. 5 μL of these RNPs was combined with the following components for a final volume of 50 μL (listed at final concentration): 10 μL of target RNaseP RNA (45 pg/uL) or no target control (“(−)”), reporter-immobilized hydrogels (10 hydrogels/uL), IB15 one pot LAMP trans-cleavage buffer, dNTPs (1 mM), RNAse inhibitor, Bsm DNA polymerase, Warmstart RTx reverse transcriptase, RNase P LAMP primer mix, and TIPP (0.5 U/uL) or water (“no TIPP”). Reactions were carried out at 55° C. for 35 minutes. Trans cleavage activity was detected with lateral flow assay strips. The supernatant for each reaction was applied to the sample pad of a lateral flow assay strip containing anti-FITC conjugate particles (colloidal gold). If trans cleavage occurred, the supernatant contained cleaved FAM-biotin-labeled reporter molecules which bound to an anti-biotin (e.g., streptavidin) target line on the lateral flow strip. The anti-FITC conjugate particles bound the FAM moiety on the reporter molecules and a target band appeared on lateral flow strips at the anti-biotin target line. If trans cleavage did not occur (as in NTC or no guide RNA reactions), the supernatant did not contain any FAM-biotin-labeled molecules, and nothing bound to the anti-biotin target line. The lateral flow assay strip also contained an anti-IgG flow control line, downstream of the anti-biotin target line, which bound to the anti-FITC moiety of the conjugate particles to confirm that the lateral flow assay functioned properly. Lateral flow strips without TIPP are shown in FIG. 165A and lateral flow strips with TIPP are shown in FIG. 165B. The presence of target RNA is indicated with (+) and the absence of target RNA is indicated with (−). As in the case with a fluorescent signal output, lateral flow assay signal was improved by the addition of TIPP.

Without being bound to any particular theory, it is believed that TIPP may be responsible for processing phosphates which are produced when the LAMP reaction uses up dNTPs. Those phosphates can take Mg2 ions out of solution, which may be cause a Cas enzyme to exhaust the remaining Mg2 quickly and cause the reaction to plateau more rapidly than would occur with additional Mg2 in the system. With the presence of TIPP, those phosphates may be processed and the Mg2+ may be released for use by a Cas enzyme, which can improve DETECTR signal strength as the Cas enzyme is able to react unhindered by a lack of Mg2.

The results show that the addition of TIPP improved signal compared to without TIPP and made detection on lateral flow strips more robust. The amount of TIPP added can be varied to allow flexibility in assay designs.

### Example 31: Isothermal Nicking Enzyme Amplification for DETECTR Reactions

This example describes isothermal nicking enzyme pre-amplification for DETECTR reactions. CRISPR-based diagnostic DETECTR reactions using loop-mediated isothermal amplification (LAMP) can be completed in as little as 30 minutes. In some cases, longer LAMP amplification may be required to achieve single copy sensitivity. A DETECTR reaction utilizing nicking enzyme amplification (NEAR) was developed to reduce amplification time relative to methods utilizing LAMP amplification. The DETECTR reaction with NEAR also showed improved sensitivity over NEAR reactions performed with a beacon. In the DETECTR reaction with NEAR, a 30 to 40 nucleotide region of a target nucleic acid was amplified using NEAR and detected using a Cas12 variant (SEQ ID NO: 17).

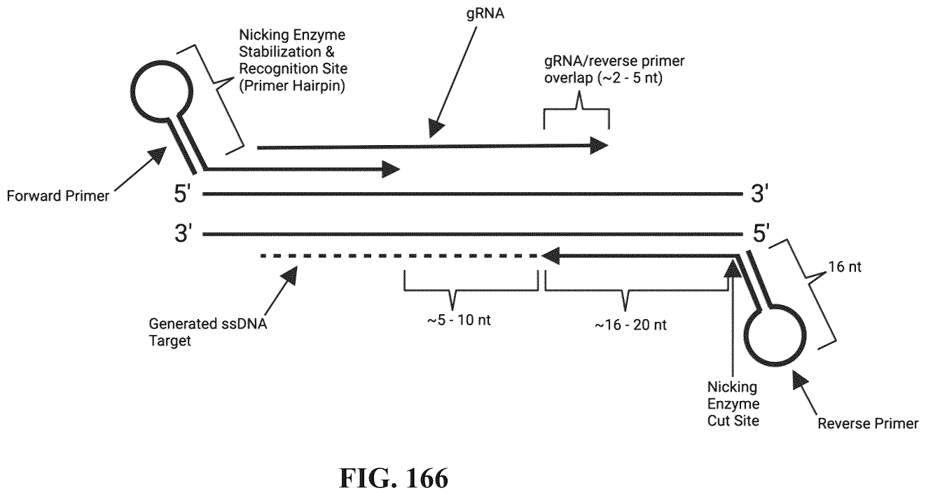

NEAR amplification was performed by contacting the target nucleic acid with a forward primer, a reverse primer, dNTPs, a DNA polymerase, and a nicking endonuclease. The reaction was performed at 60° C. for 10 minutes then cooled to 4° C. The forward primer contained a 16 to 20 nucleotide region reverse complementary to a first strand of the target nucleic acid and a 16 to 20 nucleotide nicking enzyme stabilization, binding, and recognition site 5′ of the region reverse complementary to the target nucleic acid. The reverse primer contained a 16 to 20 nucleotide region reverse complementary to a second strand of the target nucleic acid and a 16 to 20 nucleotide nicking enzyme stabilization, binding, and recognition site 5′ of the region reverse complementary to the target nucleic acid. A NEAR duplex incorporating a nicking enzyme stabilization, binding, and recognition site into an amplicon also containing the region of the target nucleic acid was then generated, as illustrated in FIG. 166. The duplex was then nicked by the nicking endonuclease and amplified using the DNA polymerase. The amplicons were detected using a DETECTR reaction with the Cas12 variant as described in at least Examples 32-35.

### Example 32: Detection of a Strep-A Target Nucleic Acid Using DETECTR with NEAR

This example describes detection of Streptococcus pyogenes using NECTR (NEAR combined with DETECTR) via amplification of the Strep-A target. The Strep-A target was amplified using the process described in Example 31. Specific conditions were optimized for Strep-A detection.

A 10 μL preamplification reaction was prepared at a final concentration of 1× Isothermal Amplification® Buffer (NEB), 1×NEBuffer™ 3.1 (NEB), 300 nM dNTPs (NEB), 4U of Bst 2.0, 3U of Nt BstNBI, 500 nM of the forward primer (SEQ ID NO: 79), and 100 nM of the reverse primer (SEQ ID NO: 80). In order to prevent non-specific amplification, the primers were added last.

8 μL of the prepared pre-amplification mixture was dispensed into a 96-well or 384-well plate. 2 μL of the target nucleic acid was added to each well in order to prevent early initiation of the reaction. Sample mixtures are mixed thoroughly and centrifuged to ensure that mixture is at the bottom of the wells. The pre-amplification reactions were incubated at 60° C. for 10 minutes and immediately placed at 4° C. or on ice to stop the reaction.

Detection of the Strep-A was achieved by our DETECTR technology via Cas12a. A Cas12a complexing reaction was prepared at a final concentration of 1× MB3, 160 nM of Cas12 variant (SEQ ID NO: 17) and 160 nM of R1107 crRNA (SEQ ID NO: 76) and incubated at 37° C. for 30 minutes. After incubation, the Beacon-AlexaFluor 594 (SEQ ID NO: 86) was added to the complexing reaction at a final concentration of 400 nM.

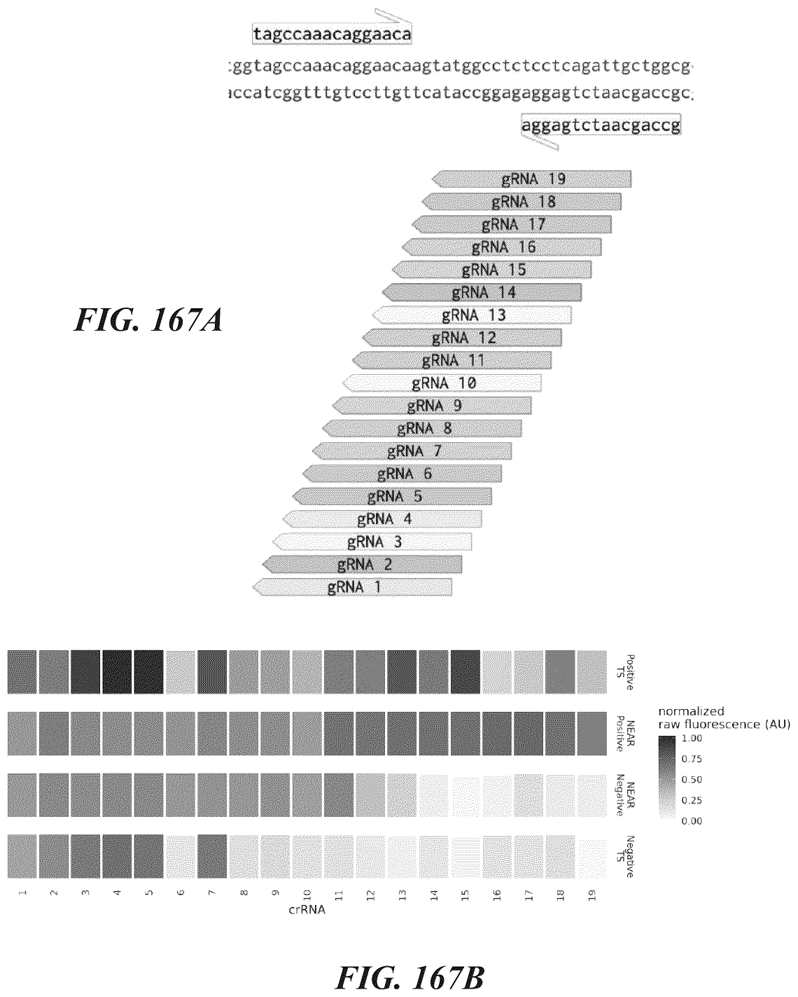

In a 384-well black assay plate on ice, 13 μL of the 1× MB3 and 5 μL of the Cas12a complexing mixture was added. Using aseptic technique to prevent any nucleic acid contamination, 2 μL of the pre-amplification reaction was added to the assay plate. The plate was sealed with an optically clear adhesive and spun at 2000 rcf. The plate was read at AF594 setting with extended gain. FIGS. 167A-167B, 169-171, and 177 show results obtained using variations of the Strep-A method described here.

TABLE 11
Nucleic acid sequences for NEAR reaction for Strep-A

| SEQ ID NO | Name | Sequence |
|---|---|---|
| SEQ ID NO: 84 | NEAR S-A | AGACTCCATATGGAGTCTAGCCAAACAGGAAC |
| | Primer F | A |
| SEQ ID NO: 85 | NEAR S-A | CGACTCCATATGGAGTCGAAAGCAATCTGAGGA |
| | Primer R | |
| SEQ ID NO: 86 | Beacon | ACAAGTATGTGAGGAGAGGCCATACTTGT |

### Example 33: Detection of a SARS-CoV-2 Target Nucleic Acid Using DETECTR with NEAR

This example describes detection of SARS-CoV-2 using NECTR (NEAR combined with DETECTR) via amplification of the E-gene target. The Strep-A target was amplified using the process described in Example 31. Specific conditions were optimized for E-gene detection.

A 10 μL preamplification reaction was prepared at a final concentration of 1× Isothermal Amplification Buffer (NEB), 1×NEBuffer 3.1 (NEB), 300 nM dNTPs (NEB), 4U of Bst 2.0, 3U of Nt BstNBI, 500 nM of the Forward primer (SEQ ID NO: 35), and 100 nM of the reverse primer (SEQ ID NO: 36). In order to prevent non-specific amplification, the primers were added last.

8 μL of the prepared pre-amplification mixture was dispensed into a 96-well or 384-well plate. 2 μL of the target nucleic acid was added to each well in order to prevent early initiation of the reaction. Sample mixtures are mixed thoroughly and centrifuged to ensure that mixture is at the bottom of the wells. The pre-amplification reactions were incubated at 60° C. for 10 minutes and immediately placed at 4° C. or on ice to stop the reaction.

Detection of the Strep-A was achieved by our DETECTR technology via Cas12. A Cas12 complexing reaction was prepared at a final concentration of 1× MB3, 160 nM of Cas12 Variant (SEQ ID NO: 17), and 160 nM of R1107 crRNA (SEQ ID NO: 76) and incubated at 37° C. for 30 minutes. After incubation, the Beacon-AlexaFluor 594 (SEQ ID NO: 81) was added to the complexing reaction at a final concentration of 400 nM.

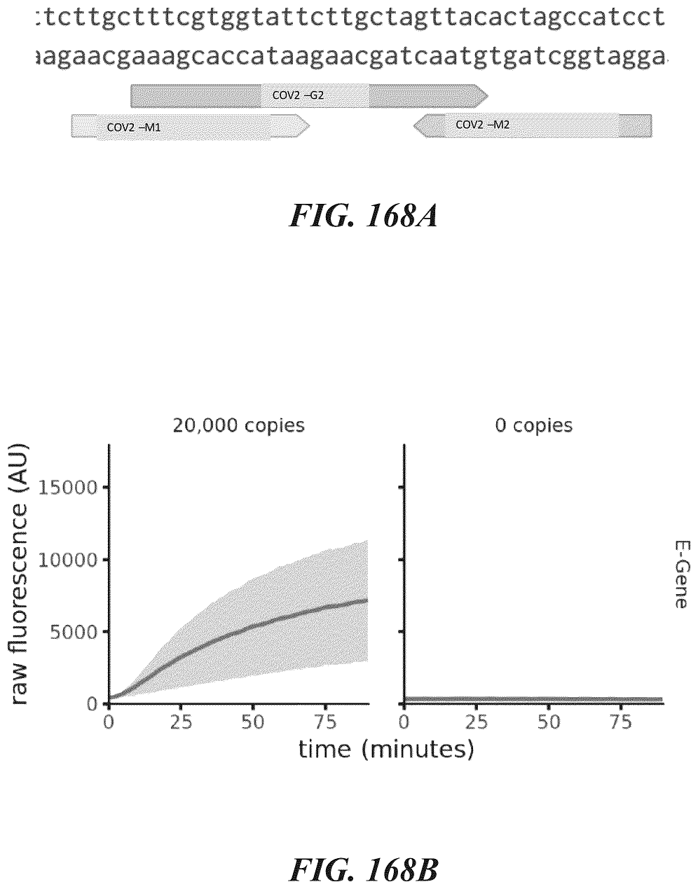

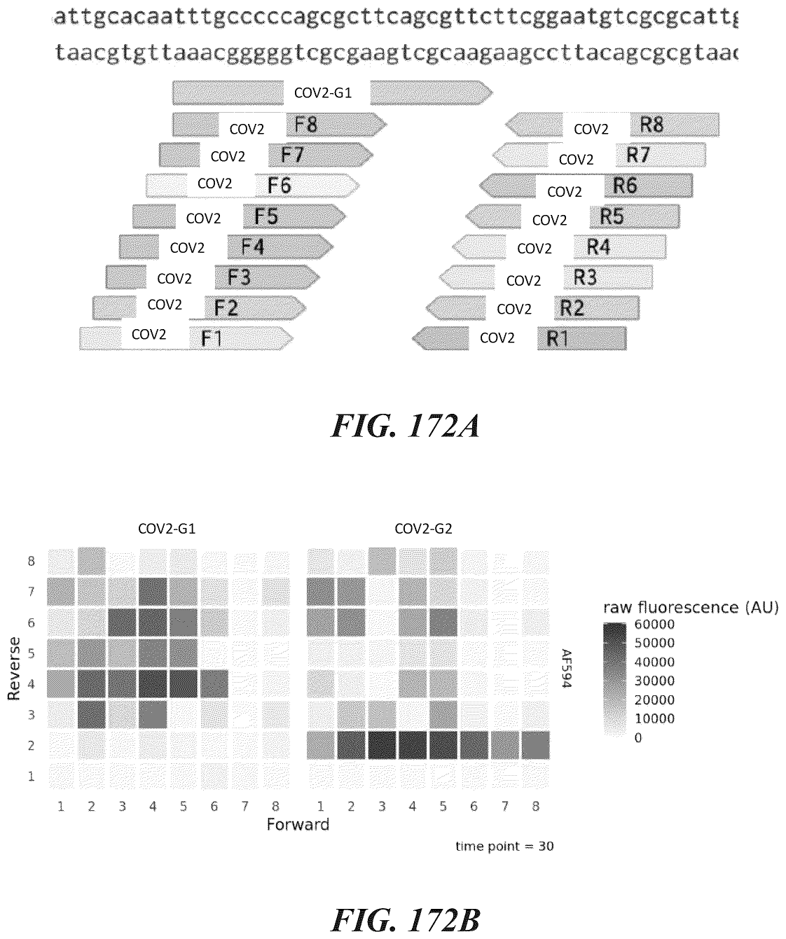

In a 384-well black assay plate on ice, 13 μL of the 1× MBuffer 3 and 5 μL of the Cas12a complexing mixture was added. Using aseptic technique to prevent any nucleic acid contamination, 2 μL of the pre-amplification reaction was added to the assay plate. The plate was sealed with an optically clear adhesive and spun at 2000×g rcf. The plate was read at AF594 setting with extended gain. FIGS. 168A-168B and 172A-176 show results obtained using variations of the SARS-CoV-2 E-gene method described here.

### Example 34: NEAR+DETECTR Reaction Optimization

### Determining the Mg2+ Concentration for a NEAR Reaction

This example describes optimization of Mg2+ concentration for the NEAR reaction. In addition, the performance of Bst 2.0 and Bst 3.0 were assessed. In this example, the protocol used was identical to that used in Example 31.

After amplification of Strep-A, the samples were tested in different buffers, Thrmopol, IsoAmp® I, and IsoAmp® II (NEB) at different added Mg2+ concentrations-0 mM, 2.5 mM, 5 mM, 7.5 mM, and 10 mM. The elapsed time at which it took to achieve a fluorescent readout from the Beacon plate reader was measured, with a lower value indicating that there were more copies of the amplicon present in the solution.

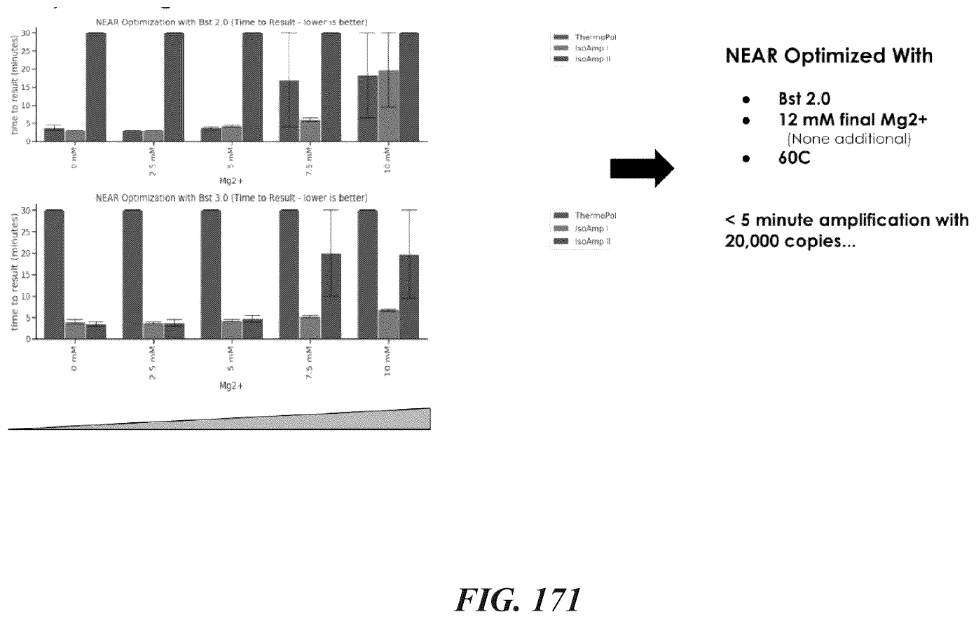

It was found that no additional magnesium was required and that Bst 2.0 was preferred for the NEAR reactions under the conditions tested. As shown in FIG. 171, Bst 2.0 was able to generate an observable fluorescent signal (˜20,000 copies) in less than 5 minutes. In FIG. 171, Bst 3.0 performed slightly slower in IsoAmp® I and IsoAmp® II buffers. Therefore, Bst 2.0 and a concentration of 12 mM Mg2+ were selected for future NEAR reaction conditions.

### Determining the Preferred Guide Sequence

This example describes how to select a guide RNA for detection of Strep-A, but it will be understood that the teachings described here may be used to select guides for other targets of interest as desired. The same primers and probe used in Example 32 were used. The following experiment determined if ssDNA could be detected using DETECTR from a NEAR reaction. Due to the biased reaction, gRNAs were generated in the reverse direction to detect any ssDNA produced from the nicking occurring on the forward primer nicking site.

A panel of 19 gRNAs was synthesized in order to determine which gRNA worked best in the DETECTR reaction under the conditions tested. As shown in FIGS. 167A-167B, all gRNAs successfully detected the positive control which comprised an oligonucleotide that was a complement of the gRNA. Guide RNAs 14-19 successfully detected the NEAR reaction products containing the target and positive control, but did not detect the NEAR negative control and negative (no input) controls. We observed that the maximum overlap with the guide RNA allowed was 3 nucleotides, otherwise signal detection in the negative controls were detected under the conditions tested.

### Characterizing NEAR RNA Targets Using DETECTR

This example describes quantification of NEAR RNA targets derived from SARS CoV-2 using the NECTR system described in Example 33. We used two NEAR primers to amplify the SARS-CoV-2 target sequence and a guide design as shown in FIG. 168A. As shown in FIGS. 168A-168B, the results of the SARS-CoV-2 E-gene DETECTR reaction are shown for samples including 20,000 NEAR copies or 0 NEAR copies. The DETECTR reaction successfully detected SARS-CoV-2 E-gene in the 20,000 target copy system and did not produce a signal in the 0 copy negative control conditions.

Next, we tried to determine the minimal incubation time of a NEAR reaction of Strep-A targets prior to detection via DETECTR. The NEAR protocol was substantially similar to that described in Example 32. Incubation of the target with NEAR reaction components proceeded for 30 seconds, 1 minute, 2 minutes, 3 minutes, 4 minutes, 5 minutes, or 6 minutes prior to DETECTR. Under the conditions tested, a minimum of 2 minutes was observed to be required to achieve a detectable DETECTR signal, however, 6 minutes was found to be more optimal as it achieved a signal much higher than the limit of detection, as shown in FIG. 169.

### Detecting NEAR RNA Targets Using Cas Variants

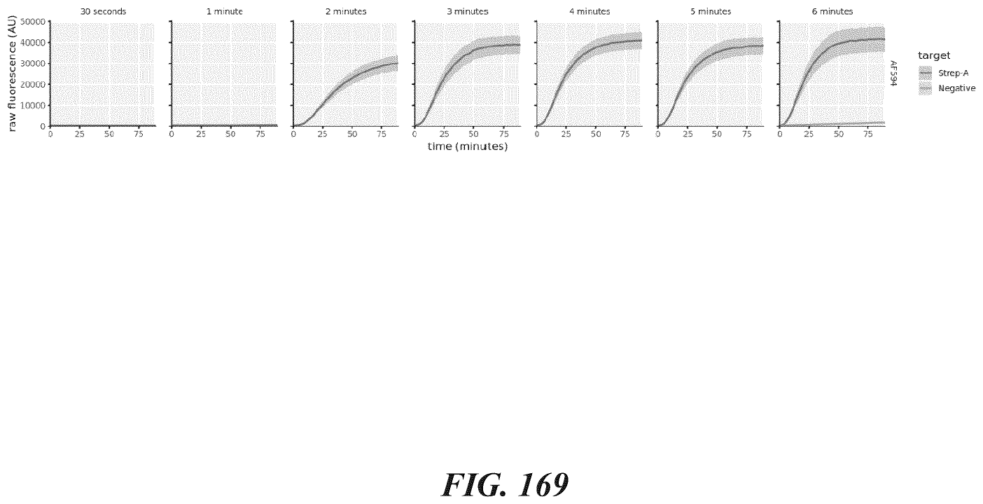

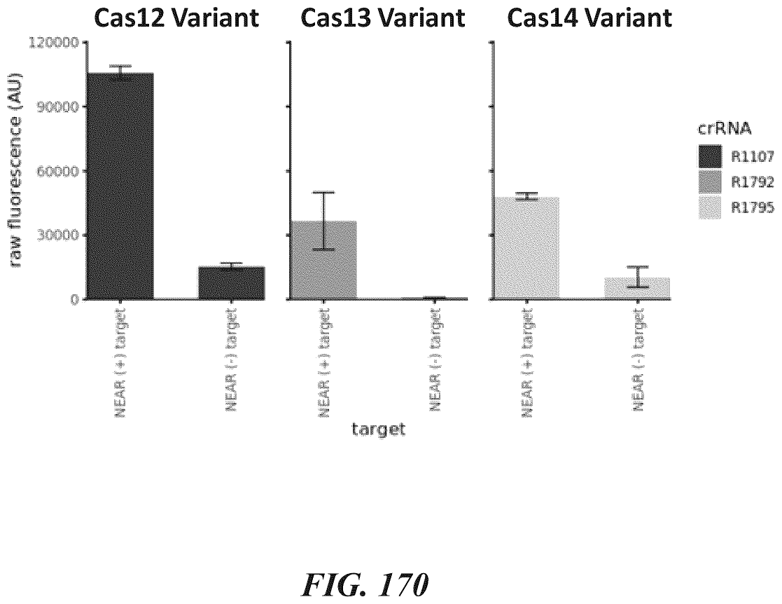

This example describes a comparison of NEAR amplicon detection using orthogonal Cas system to detect the target. The protocol of Example 32 was used for this example. Here, we utilized a Cas12 variant (SEQ ID NO: 17), a Cas13 variant (SEQ ID NO: 21), and a Cas14 variant (SEQ ID NO: 3). They were chosen because they have different cleavage preferences for the reporter molecule and can be multiplexed together. In FIG. 170, the Cas12 variant showed superior signal after NEAR DETECTR reaction under the conditions tested. These results showed that it is possible to multiplex different NEAR-DETECTR reactions with orthogonal Cas systems.

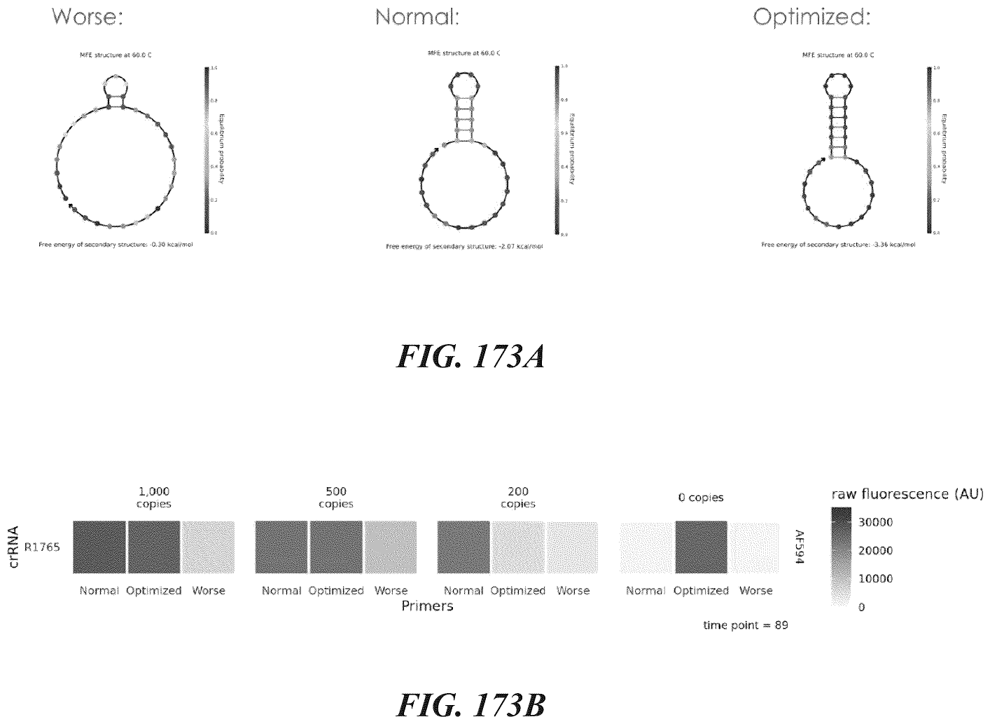

We also optimized the stability of the hairpin loop in the nickase stabilization region present in the NEAR primers. Without being bound to any particular theory, it is thought that the hairpin loop may impact the nickase activity of the enzyme. To test this, we used, alternative primer pairs to destroy, maintain, or enhance the hairpin loop formation (FIG. 173A). The performance of these new hairpin loops were tested on detection of SARS-CoV-2 E-gene as described in Example 33. In FIG. 173B, the destroyed hairpin loops showed reduced signal compared to both the normal and optimized hairpins, however, the enhanced hairpin showed strong signal even in the negative control under the conditions tested. It was determined that based on the results of this study that the normal primers would be used for future NEAR reactions.

### Determining the Optimal Reverse Transcriptase

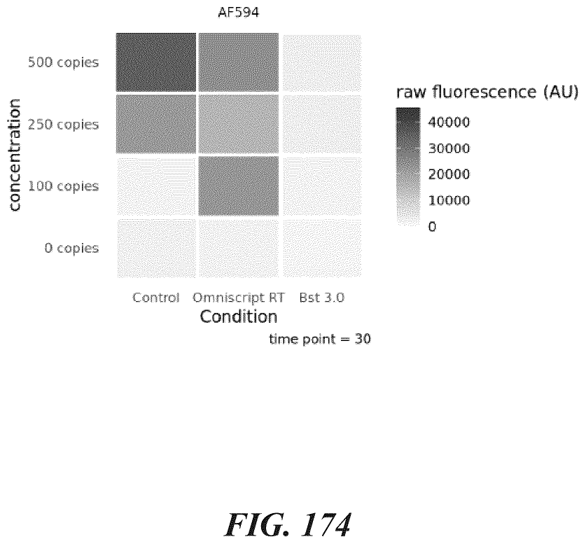

This example describes comparison of different reverse transcriptases for the RT-NECTR reaction described in Example 33. In this study, we tested Wartmstart RTx (NEB), Bst 3.0, and Omniscript RT (Qiagen) and they were compared by using the limit of detection as a metric. FIG. 174 shows that Omniscript achieved a better limit of detection compared to the other two reverse transcriptases under the conditions tested.

### Optimizing Primer Concentrations

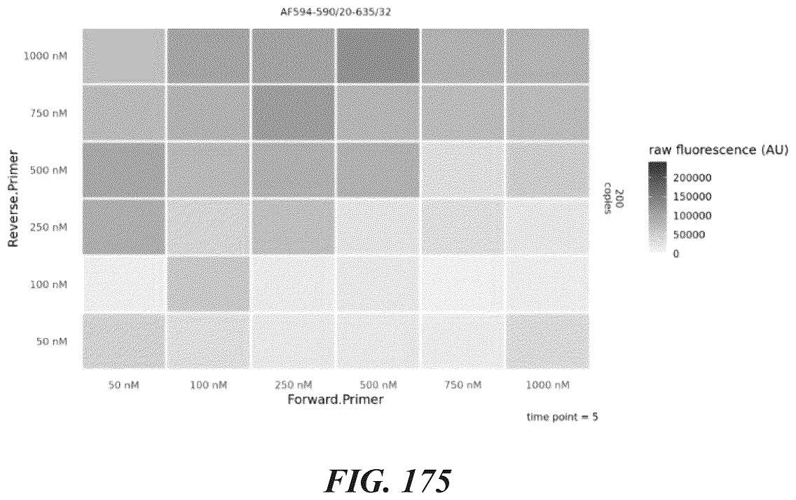

Primer concentrations were optimized in order to determine which concentrations would result in a better signal using the RT-NEAR reaction described in Example 33. In FIG. 175, primer concentrations of the forward and reverse NEAR primers were prepared at a concentration of 1000 nM, 750 nM, 500 nM, 250 nM nM, 100 nM, and 50 nM and mixed in a pairwise fashion in order to determine which concentration of forward and reverse primer will result in the best detectable signal. The forward primer used was M2805 (GAC TCC ACA CGG AGT Ctt gct ttc gtg gta tt (SEQ ID NO: 1503)) and the reverse primer used was M2811 (GAC TCC ACA CGG AGT Cgg atg gct agt gta ac (SEQ ID NO: 1504)). For this study, NEAR reactions were incubated and amplified targets for 5 minutes at 60° C. A combination of 1000 nM of the reverse primer and 500 nM of the forward primer performed best and resulted in a raw fluorescence greater than all other primer combinations tested. It was determined that by increasing the concentration of either the reverse or the forward primer resulted in increased efficiency of the reaction.

### Optimizing Amplification Times

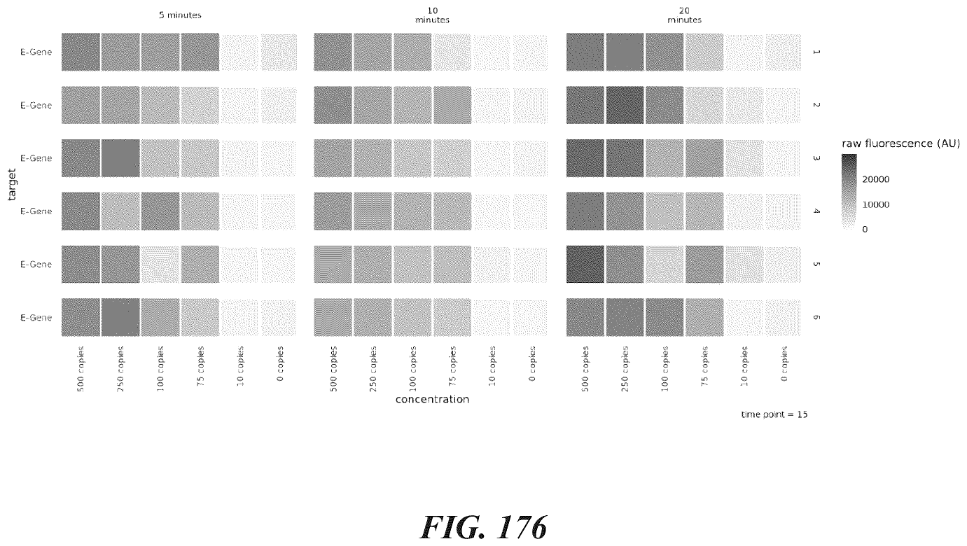

Due to the need to reverse transcribe the SARS-CoV2 prior to detection, different amplification times need to be tested in order to determine the optimal resulting signal from RT-NECTR protocol of Example 33. In FIG. 176, different amplification times of 5 minutes, 10 minutes, and 20 minutes were tested on different cDNA (SARS-CoV2 E-gene) inputs (500, 250, 100, 75, 10, and 0 cDNA copies). In this experiment, higher amplification times resulted in increased DETECTR signal, however, even after 20 minutes of incubation, DETECTR only resulted in a slight signal in 10 copies. At 75 copies, 10 minutes of amplification was sufficient to detect 75 copies of SAR-CoV2 via DETECTR within 5 minutes.

### Example 35: Testing Cas System Orthogonality

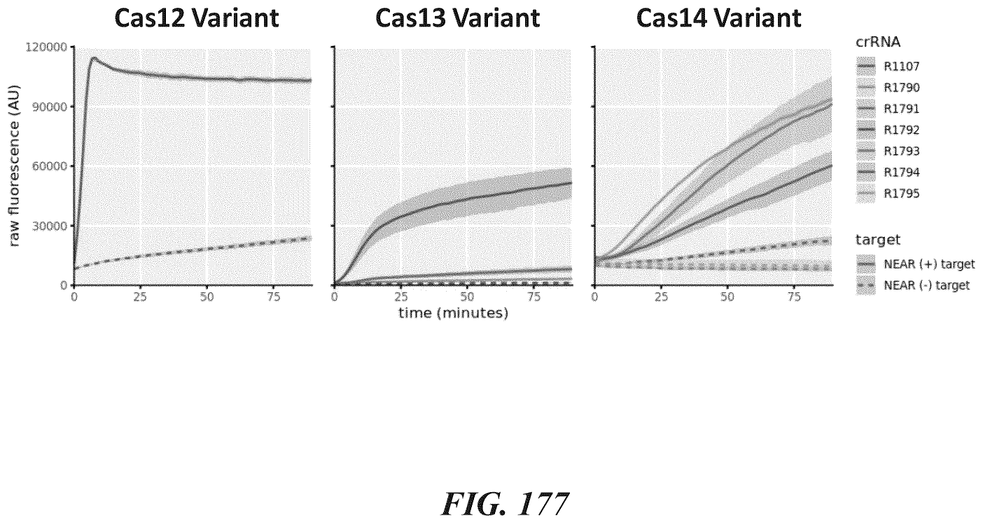

This example describes testing of different Cas systems for orthogonality of the NEAR-DETECTR reaction described in Example 32. Cas14 variant (SEQ ID NO: 3) has been previously reported to detect ssDNA, whereas Cas13 variant (SEQ ID NO: 21) has been determined to be capable of detecting ssDNA. Thus, it was determined whether it was possible for the Cas14 variant or Cas 13 variant to be used in NEAR-DETECTR to detect ssDNA. As shown in FIG. 177, Strep-A was detected using the primers M2048 and M2049, using the Cas12 variant (SEQ ID NO: 17), Cas13 variant (SEQ ID NO: 21), and Cas14 variant (SEQ ID NO: 3) systems. Detection was shown to be possible with Cas13 and Cas14 systems, suggesting the possibility of multiplexing NEAR amplicons using Cas12/13 or Cas 14/13 reaction combinations.

### Example 36: HRP-Based DETECTR Utilizing Streptavidin Beads as a Solid Support for Immobilized Reporters Comprising HRP Signal Enhancement Enzymes

The objective of the experiment described was to produce a visible signal with an HRP-based DETECTR assay, particularly in the absence of target nucleic acid amplification. In this experiment, reporters comprising HRP and biotin were immobilized onto beads functionalized with Streptavidin via the biotin-Streptavidin interaction. A schematic can be seen in FIG. 197A.

Reporter immobilization to the beads. The HRP-reporters with biotin functional groups were immobilized onto 10 μL of streptavidin beads at reporter concentrations of 1 nM, 250 nM and 500 nM respectively.

HRP-based DETECTR Assay. To each of the three separate bead suspensions, at 1 nM reporter, 250 nM reporter and 500 nM reporter, respectively, the programmable nuclease Cas13 having SEQ ID NO: 21, complexed with the nucleic acid guide, and 4 nM of target was added and allowed to react at 37° C. for 30 minutes. In this step, the HRP portion of the reporter was released into solution via cleaving by the activated programmable nuclease-guide nucleic acid-target complex. The supernatant containing the cleaved HRP was then decanted from the bead suspension and combined with a solution containing the HRP enzyme substrate, TMB, at an approximate ratio of 2:1 (v/v) TMB to HRP supernatant. The HRP supernatant was first transferred to a fresh 96-well clear plate and then TMB was added to each well at approximately T=0. It takes about 20-30s from adding the TMB to starting the absorbance read on the plate reader. While the HRP enzyme consumed the substrate, TMB, the absorbance of the solution was measured. Results can be seen in FIGS. 197B-197D.

An HRP-based DETECTR Assay was performed at six target concentrations to determine a limit of detection (LOD). Six aliquots were taken from the bead suspension functionalized with HRP reporter at 250 nM. A Cas13 programmable nuclease complexed with guide nucleic acid was added to all six. Target was added to each of five aliquots to obtain target concentrations of 700 attM, 7 fM, 70 fM, 700 fM and 350 μM. No target was added to the sixth aliquot to act as the no-target control (NTC). Reactions in all six aliquots were carried out at 37° C. for 30 minutes. The supernatant containing the cleaved HRP was then decanted from the bead suspensions and separately combined with solutions containing the HRP enzyme substrate, TMB, at an approximate ratio of 2:1 (v/v) TMB to HRP supernatant. While the HRP enzyme consumed the substrate, TMB, the absorbance of the solutions was measured. Results can be seen in FIG. 197E. An LOD in the mid fM range was determined under the conditions tested.

### Example 37: HRP-Based DETECTR Utilizing Streptavidin-Coated Plates as a Solid Support for Immobilized Reporters Comprising HRP Signal Enhancement Enzymes

This example describes effects of different concentrations of reporter functionalized to a streptavidin-coated plate, and measurements to determine the limit of detection.

Reporter immobilization. The HRP-reporter, rep 194, as described herein, containing a biotin functional group was immobilized onto streptavidin plates in three separate aliquots, where the concentrations of reporter was 5 nM, 50 nM and 250 nM respectively. A schematic of the reporter can be seen in FIG. 198D.

HRP-based DETECTR Assay. To each of the three separate plates, at 5 nM reporter, 50 nM reporter and 250 nM reporter, respectively, a programmable nuclease Cas13, complexed with the nucleic acid guide, and 10 μM of target was added and allowed to react at 37° C. for 30 minutes. In this step, the HRP portion of the reporter was released into solution via cleaving by the activated programmable nuclease-guide nucleic acid-target complex. The supernatant containing the cleaved HRP was then decanted from the streptavidin-coated plates and combined with a solution containing the HRP enzyme substrate, TMB. While the HRP enzyme consumed the substrate, TMB, the absorbance of the solution was measured. Results can be seen in FIGS. 198A-C.

An HRP-based DETECTR Assay was performed at five target concentrations to determine a limit of detection (LOD). Five wells of a streptavidin-coated plate were functionalized with HRP reporter at 250 nM. Cas13 programmable nuclease complexed with guide nucleic acid was added to all five. To each of four wells, target was added to obtain target concentrations of 10 fM, 100 fM, 1 μM and 10 pfM. No target was added to the fifth well to act as the no-target control (NTC). All five conditions were allowed to react with the HRP-reporter-bound streptavidin-coated plates at 37° C. for 30 minutes. The supernatant containing the cleaved HRP was then decanted from the plates and combined with solutions containing the HRP enzyme substrate, TMB. While the HRP enzyme consumed the substrate, TMB, the absorbance of the solutions was measured. Results can be seen in FIG. 198D. An LOD of about 10 μM was determined in the 50 nM HRP-reporter bound wells under the conditions tested. An image of six wells of a plate containing the solutions analyzed in FIGS. 198A-198C can be seen in FIG. 198E, where the left-hand column contains eluate from the HRP-DETECTR reaction run with 10 μM target present, and the right-hand column contains eluate from the HRP-DETECTR reaction run without target present (no target control, NTC).

### Example 38: HRP-Based DETECTR Assay Utilizing Hydrogels

This example describes an HRP-DETECTR assay utilizing HRP-hydrogels comprising different PEG-DA ratios.

HRP-Hydrogel Prep. Preparation of HRP-hydrogels was similar to that described in Example 25 above. 22.5 μL of PEG/PEG-DA monomer mix was added to a PCR tube with 2.5 μL of reporter for a final concentration of either 10 μM or 1 μM reporter in 25 μL hydrogel mix. The mixture was vortexed and spun down briefly, then pipetted into a PDMS microfluidic channel on a glass slide. The hydrogel mixture was polymerized by exposure to 365 nm UV light (275 ms for 20% PEG-DA, 260 ms for 35% PEG-DA). Hydrogels were collected from the PDMS channel, collected, washed, resuspended, spun down, and supernatant removed, leaving about 100 μL of hydrogel. Hydrogels were stored in 1 mL of wash buffer at 4° C. overnight. The concentration of each hydrogel pool was determined by pipetting 1 μL onto a glass slide and counting particles under a microscope. Volume was adjusted to reach the desired final concentration.

HRP-Hydrogel DETECTR assay. Detection assays were performed using different PEG-DA concentrations (20% or 35%) co-polymerized with different concentrations of reporters (10 uM or 1 uM) comprising HRP-based detection moieties and acrydite functionalities (rep188). Samples included 4 nM of target, a no-template control (NTC), and an off-target control (OTC). The OTC sample included a nucleic acid with a different sequence from the target nucleic acid, and served as a control for the specificity of the reaction in that no signal was expected in response to the OTC. One of the reactions did not include any reporter (a “no rep control”). Two replicates were run for each condition (“prep 1” and “prep 2”).

The reactions were run as follows. 5 μL of hydrogel were aliquoted into single PCR tubes. Programmable nuclease detection complexes were prepared by combining 3.6 μL of nuclease-free water, 1 μL of 5× buffer, 0.08 μL of gRNA, 0.32 μL of 5 μM Cas enzyme SEQ ID NO: 34, and incubating at 37° C. for 20-30 minutes. 26 μL buffer was added to the PCR tubes containing hydrogels, along with 5 μL of detection complexes, and 4 μL of sample (i.e., target, NTC, or OTC). The final concentrations of components in the reaction were 40 nM gRNA, 40 nM Cas enzyme, and 4 nM target. The mixtures were vortexed, spun down briefly, and incubated for 30 minutes at 50° C. with no shaking. The reactions were stopped by placing on ice. Tubes were spun down for 5 minutes, and 10 μL of supernatant from each tube was pipetted to separate wells of a 96-well plate. 20 μL of TMB was added to the wells, and absorbance was read on a plate reader. Results for absorbance are illustrated in FIGS. 199A-199F.

### Example 39: HRP-DETECTR on NHS Activated Resin

In this example, an HRP-DECTER assay is demonstrated by immobilizing HRP-reporter onto NHS-activated resin.

HRP-reporter immobilization to NHS-activated resin. Columns were prepared and the assay were run according to the schematic illustrated in FIG. 200A. The columns were prepared by adding 400 μL of 1 nM reporter directly to the dry resin, and incubating at room temperature for 1 hour with end-over-end mixing. The top and bottom caps were removed, and the columns were placed in 2 mL collection tubes. The columns were centrifuged at 1000×g for 1 minute. The columns were then washed with 500 μL wash buffer (1× PBS+0.1% TWEEN-20) and centrifuged at 1000×g for 1 minute. This wash was repeated three times. 500 μL 1M ethanolamine was added to quench the remaining active sites, the top and bottom caps were replaced, and the contents mixed by end-over-end mixing for 15-20 minutes. The top and bottom caps were removed, the columns placed in 2 mL collection tubes, and centrifuged at 1000×g for 1 minute. The flow through was discarded. 500 μL was buffer was added, the columns centrifuged at 1000×g for 1 minute, and wash steps repeated three times. 400 μL wash buffer was added for storage upright overnight at 4° C. The columns were spun down to wash the overnight supernatant by centrifugation at 1000×g for 1 minute. The columns were washed three times with 500 μL wash buffer. 50 μL TMB was added to the last wash to make sure it did not turn blue. The columns were then ready for the assay.

HRP-DETECTR assay. The detection reagents were prepared by combining 313.2 μL nuclease free water (NFW), 78.4 μL 5× buffer, 0.08 μL crRNA, 0.32 μL of 5 μM Cas enzyme SEQ ID NO: 34, and the resulting 392 μL were added to the resin in the columns. Columns then received either 8 μL of 50 nM target or NFW, and were incubated on a thermomixer at 48° C. for 45 minutes at 500 RPM with intermittent shaking (15 seconds on, 2 minutes off). The columns were spun down and eluate collected. The columns were washed four times with 400 μL buffer, and the wash fractions were collected. 150 μL of eluate was added to wells of a clear 96-well plate. 50 μL of substrate (TMB) was added to each well and absorbance (optical density at 650 nm) was measured. Illustrative results are shown in FIGS. 200B-200E. A top-down image of the plate is shown in FIG. 200F. Wells 20007, 20008, 20009, and 20010 represent wash 1 for +target, eluate for +target, wash 1 for NFW, and eluate for NFW, respectively.

### Example 40: HRP DETECTR Limit of Detection Study Utilizing Poly HRP

This example describes an HRP-DETECTR assay the demonstrated 100 fM limit of detection utilizing a poly-HRP reporter.

Preparation of reporter resin. Carboxylic beads having a concentration of about 10 mg/mL were functionalized with a poly-HRP reporter, as illustrated in FIG. 196. At the concentrations and amounts used, there were approximately 14× 10⁹ reporter molecules per gram of beads.

HRP-DETECTR assay. Two different volumes of the reporter poly-HRP reporter conjugated beads were tested, 2 μL and 5 μL. A programmable nuclease having SEQ ID NO: 34 was complexed with guide nucleic acid and added to the beads. Reactions also included 10 fM, 100 fM, 0.5 pM, 1 pM, 10 pM, 100 pM or 1 nM of target, except for a no template control (NTC). The contents of the columns were then reacted for 45 minutes at 50° C. The liquid portion was then collected and added to a TMB solution. The absorbance of the mixture was measured over time, and results are illustrated in FIGS. 201A-201D.

### Example 41: Sample Lysis with Proteinase K

This example describes a method of lysing a sample with a protease to release nucleic acids so that they are accessible to a programmable nuclease (e.g., in a DETECTR reaction).

A sample comprising cells and/or viral particles is exposed to a lysis buffer solution comprising Proteinase K to digest the proteins (including RNAses, DNAses, matrix proteins, viral proteins, etc.) in the sample and release any nucleic acids therefrom. The sample is incubated in the lysis buffer solution at 37° C. for a pre-determined digestion time (e.g., 1 hour) to allow for proteolysis to occur. A serine protease inhibitor (e.g., PMSF) is then added and the mixture is incubated at 37° C. for a pre-determined inhibition time (e.g., 1 hour) to inactivate the Proteinase K. The programmable nuclease and additional detection reagents (e.g., guide nucleic acids, reporters, etc.) are then added to a mixture comprising the released nucleic acids and inactivated Proteinase K.

### Example 42: Signal Amplification with NucC

This example describes a method of detecting a target nucleic acid using a programmable nuclease, a guide nucleic acid, a first reporter, an enzyme, and a second reporter. In general, formation of a complex comprising the programmable nuclease, the guide nucleic acid, and the target nucleic acid activates the programmable nuclease (a Cas protein, in this example). The activated Cas protein cleaves the first reporter, releasing a first nucleic acid section (a cyclic adenylate, in this example), which activates the enzyme (NucC, in this example). NucC then cleaves the second reporter, which separates a fluorescent label from a quencher. Detection of the fluorescent label is used as an indicator for presence of the target nucleic acid in the sample.

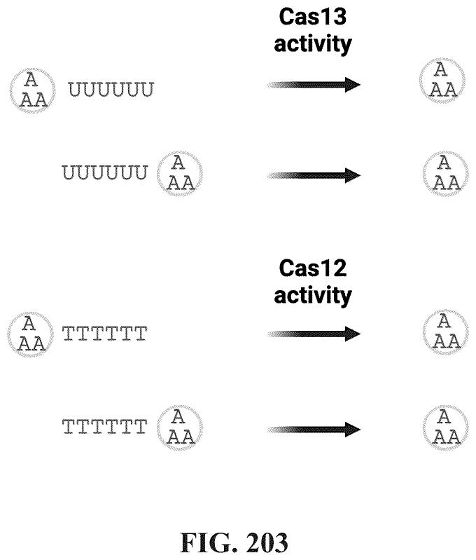

In this detection scheme, Cas13 removes a blocking group from a linear RNA (blocked-activator). The blocking group is removed specifically due to the cleavage preferences of Cas13 for U's over A's. The shorter activator is now able to function to activate NucC. NucC then cleaves a dsDNA reporter molecule which generates a fluorescent or colorimetric signal. The Cas13 is capable of multiple turnovers and so is the NucC. The activity of these two proteins together generates a signal amplification effect, where the initial Cas13 detection of a pathogen or other nucleic acid sequence of interest is finally reported out by NucC. An illustrative schematic of this system is illustrated in FIG. 202, and illustrative first reporters are shown in FIG. 203 (poly-U based reporters for Cas13, poly-T based reporters for Cas12). Specific examples of first reporters (mod286, mod287, mod288, and mod289) and strands of a double-stranded second reporter (rep213 and rep214) are provided in Table 12 below.

TABLE 12
Exemplary first reporters and exemplary double-stranded second reporters

| Name | Sequence | Description |
|---|---|---|
| rep213 | /56-FAM/ | Top strand of dsDNA reporter, from White |
| | AGTGTTACATTATCCACCAT | lab |
| | GGCGAGCTTT (SEQ ID NO: | |
| | 1505) | |
| rep214 | AAAGCTCGCCATGGTGGATA | Bottom strand of dsDNA reporter from |
| | ATGTAACACT/3IABKFQ/ | White lab |
| | (SEQ ID NO: 1506) | |
| mod286 | rArArArUrUrUrUrUrUrU | Unmodified NucC blocked-activator |
| | (SEQ ID NO: 1507) | |
| mod287 | /52FA/rArArUrUrUrUrUrUrU | Modified NucC activator with fluorobase |
| | (SEQ ID NO: 1508) | in the first position |
| mod288 | rA/i2FA/rArUrUrUrUrUrUrU | Modified NucC activator with fluorobase |
| | (SEQ ID NO: 1509) | in the second position |
| mod289 | rArAr/i2FA/UrUrUrUrUrUrU | Modified NucC activator with fluorobase |
| | (SEQ ID NO: 1510) | in the third position |

### High-Temperature NucC Tolerance

The temperature tolerance of NucC was assessed in order to determine whether NucC retains its ability to cleave double-stranded DNA at temperatures above 37° C. and is compatible with DETECTR assays run at elevated temperatures with a thermostable programmable nuclease (e.g., a Cas13c, etc.). Cleavage assays were performed with Vibrio metoecus NucC (VmeNucC) in MB3 buffer with FQ double-stranded DNA reporter (rep213+214). The final concentration of activator (cyclic triadenylate activator) was 1 nM and the final VmeNucC concentration was 250 nM.

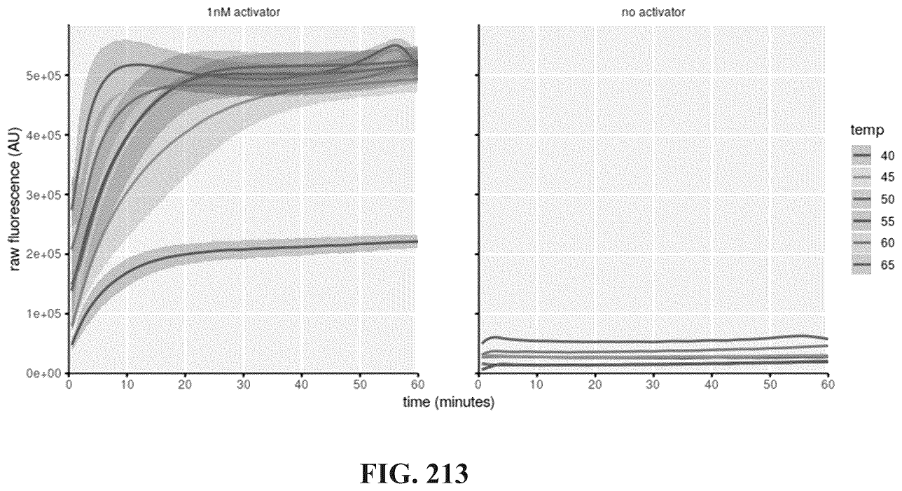

Each reaction was run for 60 minutes at a different temperature: 40° C., 45° C., 50° C., 55° C., 60° C., or 65° C. In parallel, a second series of reactions at the different temperatures were performed in the absence of the 1 nM activator. Graphical representations of the results with and without activator present during the reactions are shown in FIG. 213. It was found that VmeNucC was able to robustly cleave the double-stranded DNA reporter in the presence of 1 nM activator at all temperatures tested, making it a viable candidate for use in an elevated temperature signal amplification assay as described herein.

Activation of NucC Orthologs with Uncapped Linear Activators

The activity of NucC after addition of a linear activator was assessed in order to determine whether NucC could be activated by linear oligos, in addition to cyclic oligos, as shown in FIG. 202, which would greatly simplify manufacturing and design of appropriate activators. Cleavage assays were performed with VmeNucC and another NucC ortholog VmeNucC in MB3 buffer with FQ double-stranded DNA reporter (rep213+214). The final concentration of activator (uncapped mod286 with all adenosine residues removed by earlier Cas13 treatment) was 1 μM, 100 nM, 10 nm, 1 nM, 100 μM, or 0 nM and the final VmeNucC or FcoNucC (NucC from Faecalicatena contorta 2789STDY5834876) concentration was 250 nM.

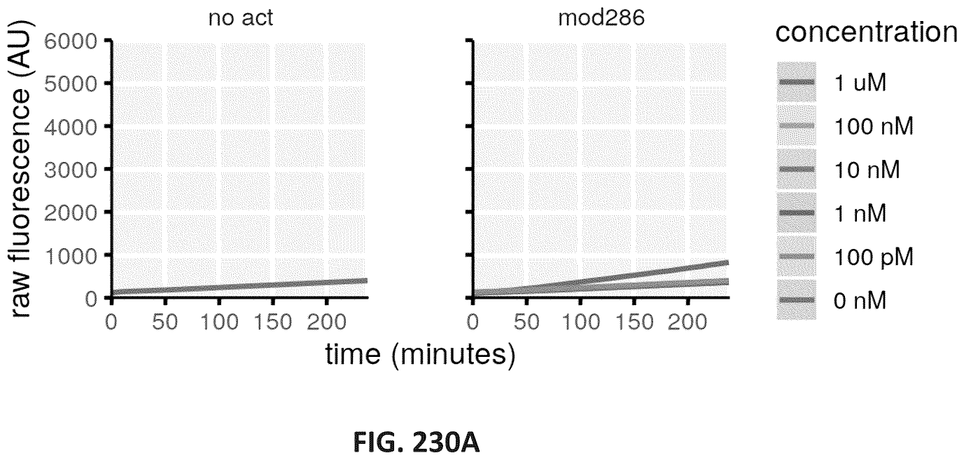

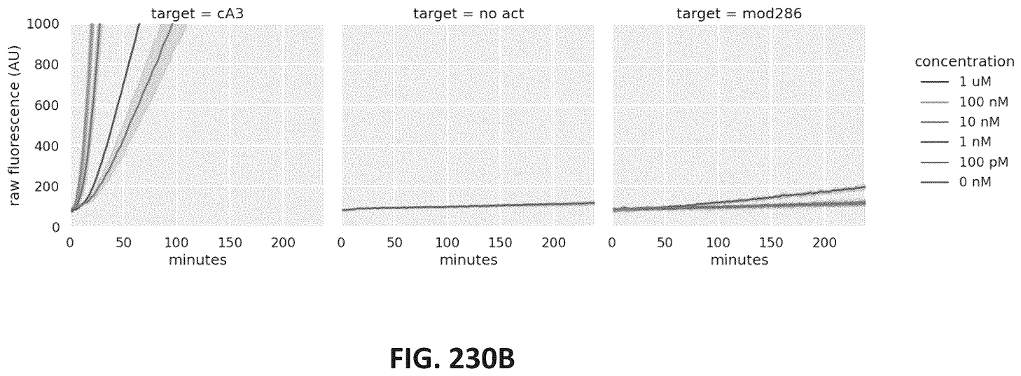

Each reaction was run for 240 minutes with or without the presence of the linear activator at various concentrations. Graphical representations of the results with and without linear activator present during the reactions are shown in FIGS. 230A-230B. It was found that both VmeNucC and FcoNucC were able to be activated by the linear activator (uncapped mod286) under the conditions tested.

### Example 43: Signal Amplification Systems with HRP

This example describes results for several approaches to signal amplification of Cas-based detection of target nucleic acids using horseradish peroxidase (HRP). Systems included those based on surface-tethered HRP, magnetic bead-tethered HRP, magnetic bead capture of HRP, and processes that included an HRP concentration step.

Surface Tethered Approach: HRP was bound to the surface of functionalized plates (e.g., streptavidin, NHS, epoxy, etc.) by a DNA or RNA tether; details are specified in the results section below. Wells were washed 4× with wash buffer. After the final wash, Cas reaction buffer was added to each well, along with the Cas-gRNA complex. Target was then added to the reaction wells. Reactions were allowed to proceed at 37° C. for 30 minutes. The supernatant was then transferred to a second plate which contained the HRP substrate, TMB. Absorbance was then measured for 30 minutes at 37° C.

Magnetic Bead Tethered Approach: This process was similar to the surface-tethered approach, but the HRP was bound to the surface of functionalized magnetic beads. After incubating with activated Cas-gRNA complex at 37° C. for 30 minutes, the magnetic beads were pelleted with a magnet, and the supernatant was transferred to a plate containing the HRP substrate, TMB. Absorbance was then measured for 30 minutes at 37° C.

Magnetic Bead Capture Approach: HRP-biotin reporter was incubated in-solution with activated Cas complex at 37° C. for 30 minutes. Magnetic beads were then added to capture the intact reporter and allow HRP reporters that had been cleaved by the Cas complex to remain in solution. The supernatant was then transferred to a plate containing the HRP substrate, TMB. Absorbance was then measured for 30 minutes at 37° C.

HRP Concentration Approach: Anti-HRP antibodies were attached to agarose resin and the resin was loaded into an empty micro-spin column. Following the Magnetic Bead Tethered Approach or Magnetic Bead Capture Approach, the HRP-containing supernatant was transferred to the prepared column and incubated at ambient temperature for 30 minutes. The column was centrifuged and the flow through was discarded. The HRP substrate, TMB, was added to the column and incubated for 30 minutes at room temperature and then recovered by centrifugation. Stop Solution was then added to the column and recovered by centrifugation. The recovered TMB and Stop Solution were combined and the absorbance was measured.

### Evaluation of In-Solution HRP Reporter

Surface-tethered reporters provide various advantages detailed herein. However, in some early experiments, detection below 1 pM (about 15 million copies per reaction) was challenging in some implementations. In an effort to improve sensitivity for lower target concentrations, we redesigned the assay so that the HRP reporter would be cleaved in-solution and then the reporter captured onto magnetic beads. Without wishing to be bound by theory, it was believed that the Cas protein would have easier access to reporter targets in solution than those bound to a surface. An example illustration of the assay workflow is provided in FIG. 202.

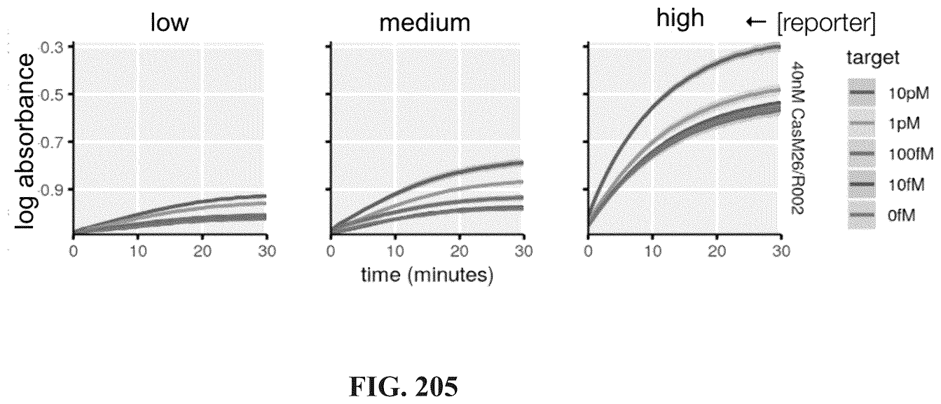

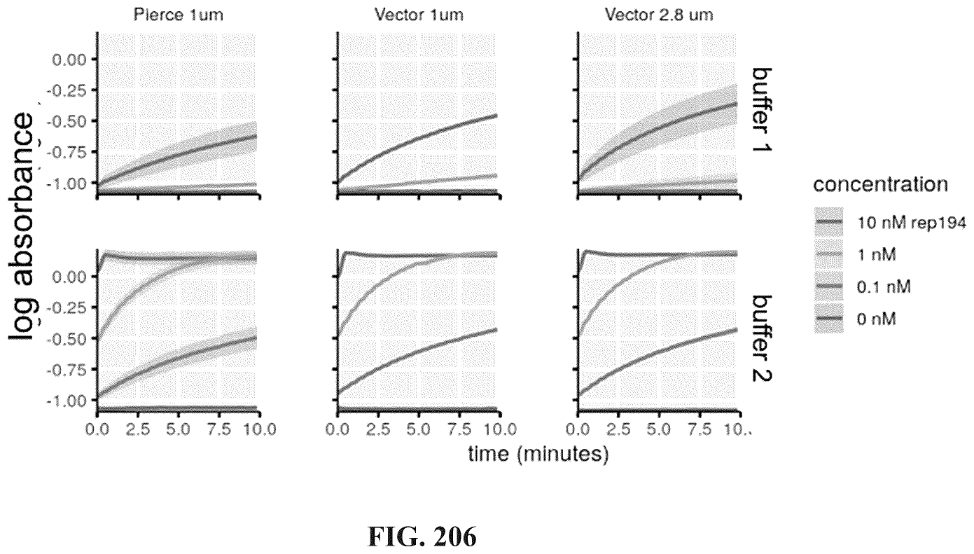

We evaluated the impact of reporter concentration on the performance of the in-solution assay (FIG. 205) and the binding buffer for capturing the reporter (FIG. 206). These experiments revealed an optimal concentration of reporter to be captured by the magnetic beads (labeled “medium” in FIG. 205) that enabled detection down to at least 100 fM (1,500,000 copies per reaction) under the conditions tested. Lower reporter concentrations appeared to make the assay less sensitive and higher reporter concentrations increased the background to the point that differentiation between positive and negative reactions was more difficult. In this example, reporter concentrations of 0.5 nM, 1 nM, and 5 nM were designated “low,” “medium,” and “high,” respectively (see also FIG. 205).

We also evaluated the choice of magnetic bead and the binding buffer for the reporter (FIG. 206). This revealed that while multiple brands and sizes of magnetic beads perform similarly for HRP-biotin capture, the binding buffer can impact the ability to reduce the background of the assay. As shown in FIG. 206, Buffer 2 (a Cas activity buffer MBuffer1) reduces the ability of the beads to bind the biotin-HRP, whereas Buffer 1 (a manufacturer suggested binding buffer of phosphate buffered saline) was able to efficiently pull-down the reporter.

### Optimization of Magnetic Beads

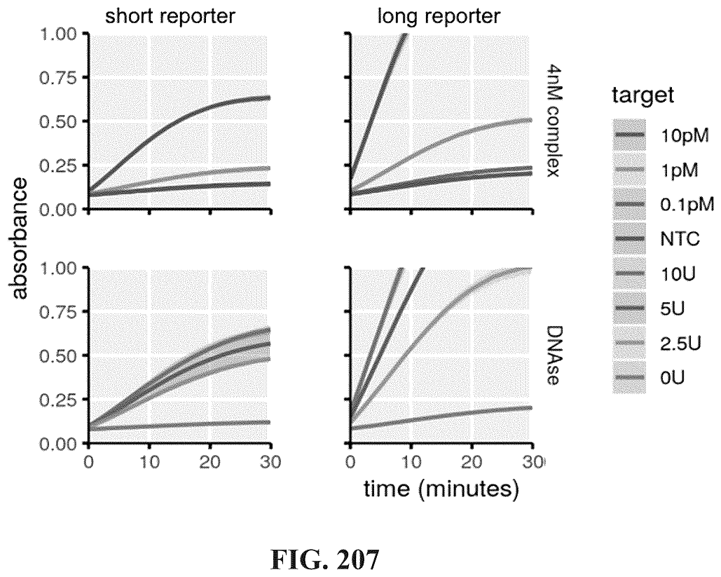

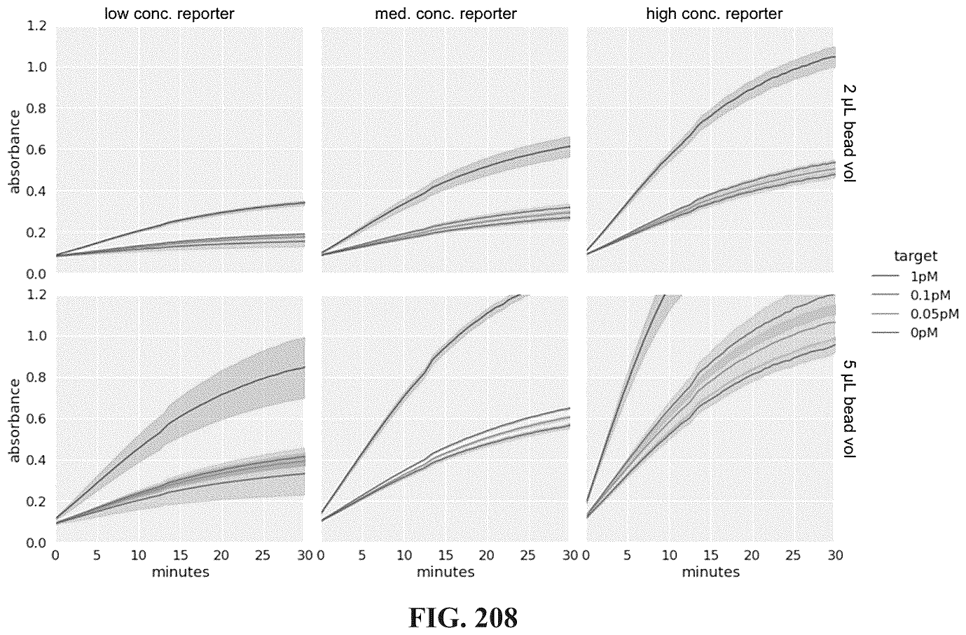

As an alternative to the in-solution HRP reporter followed by capture, we also evaluated the possibility of reducing potential steric inhibition of the reporter cleavage reaction by increasing the length of the tether to better accommodate the size of the Cas protein. Instead of using functionalized plates or slides, we used magnetic beads, as we found these easier to manufacture and also increased the surface area available for the Cas to interact with while active. Our experiment demonstrated that a shorter reporter than we used initially showed lower signal and lower sensitivity than a longer reporter that was designed to allow for better access of the Cas protein to the cleavable region (e.g., single strand RNA region) (FIG. 207). The longer reporter included a 36-carbon spacer on the 5′ end and a 54-carbon spacer at the 3′ end, and is represented as follows (with iSp18 denoting 18-carbon spacers): 5BiotinTEG//iSp18//iSp18/TTT TTT TTT rUrUrU rUrUT TTT TTT TTT T/iSp18//iSp18//iSp18//3HRPMD/(SEQ ID NO: 1511). The shorter reporter lacked spacers, and is represented as follows: 5′ HRP-TTT TTT TTT TTT rUrUrUrUrU-TTT TTT TTT TTT-3′ Biotin-TEG (SEQ ID NO: 1512). The longer reporter increased the sensitivity of the assay and increased the signal at each target concentration without dramatically increasing the background. We next optimized the concentration and the reporter bound to the beads during their functionalization and the volume of the beads added to the DETECTR® reaction. As illustrated in FIG. 208, results showed that an optimized combination of bead concentration and bead volume enabled detection down to at least 50 fM (750,000 copies per reaction) under the conditions tested. Here, bead concentrations of 50 nM, 100 nM, and 200 nM were designated “low,” “medium” and “high,” respectively.

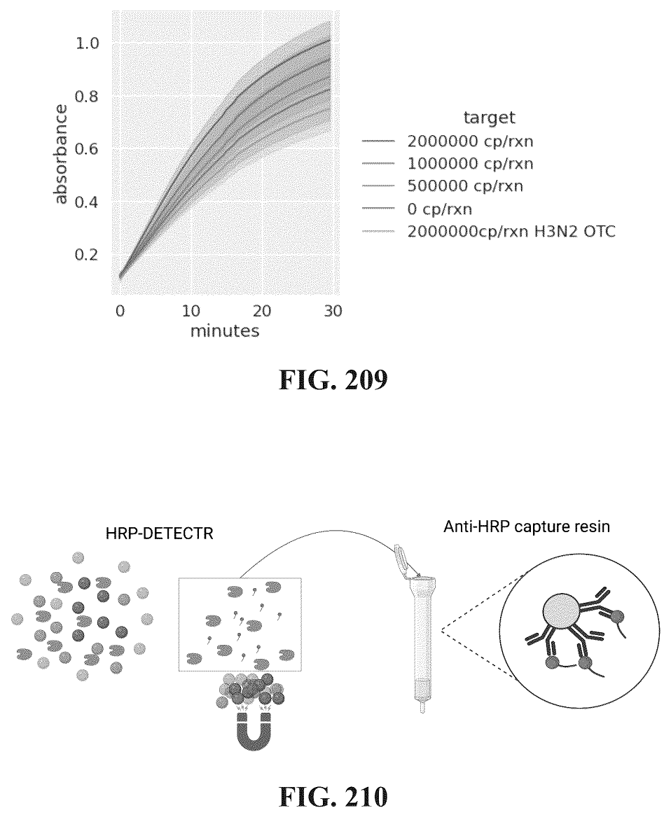

To evaluate the performance of the assay with the optimized reporter and bead concentrations, we performed a 22 replicate experiment with different concentrations of target nucleic acid, expressed in copies per reaction (“copies/rxn” or “cp/rxn”). Concentrations tested included 2,000,000 copies/rxn, 1,000,000 copies/rxn, 500,000 copies/rxn, and 0 copies/rxn, using ddPCR quantified synthetic SARS-CoV-2 RNA from Twist. Illustrative results are shown in FIG. 209. To demonstrate the specificity of the assay we also included 2,000,000 copies/rxn of FluA H3N2 synthetic RNA from Twist as an off-target control (OTC). The results of the experiment showed differentiation from both the 0 copies/rxn and 2,000,000 copies/rxn OTC conditions at 500,000 copies/rxn under the conditions tested. Differentiation from background was stronger at 2,000,000 copies/rxn and 1,000,000 copies/rxn.

### Optimization of Magnetic Beads

In the experiments discussed above in this example, the effective TMB and HRP concentrations were constrained by the volumes used for the DETECTR® reaction and the binding buffers. We hypothesized that capturing the HRP onto a surface after the cleavage reaction but before signal detection could provide better control over the TMB-HRP reaction kinetics.

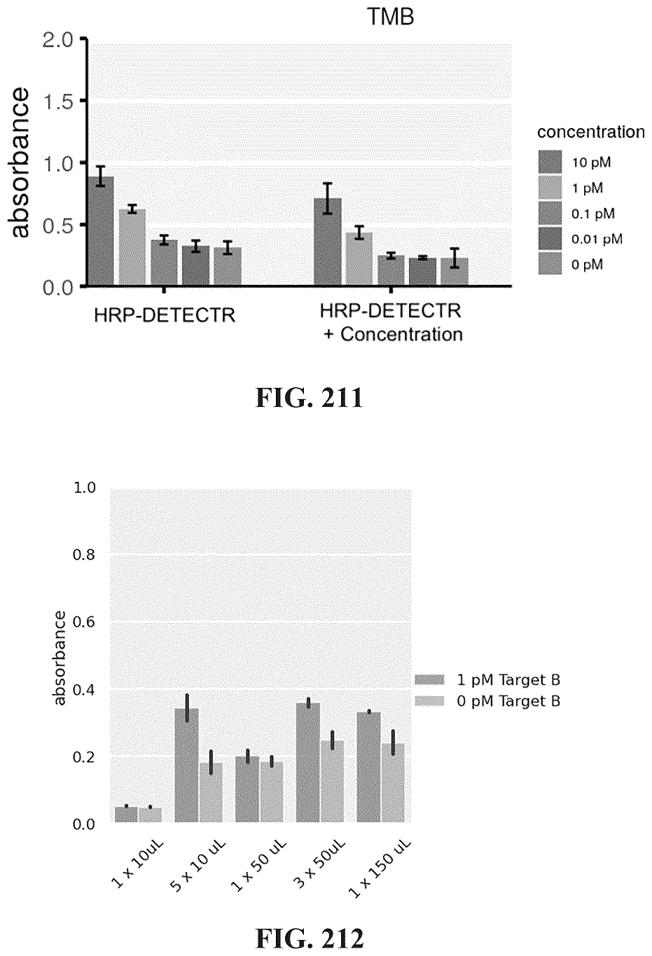

FIG. 211 illustrates results of an experiment in which we compared the performance of the concentrated HRP to the regular HRP+DETECTR® reaction. However, we found that the added concentration step did not dramatically improve the performance of the assay. We evaluated several reasons for this result, including testing the performance of HRP and antibody bound HRP, which showed a decrease in HRP function when bound by an antibody (data not shown). Furthermore, we suspected that the TMB may not have sufficient time to interact with the HRP in the anti-HRP resin. We next evaluated whether adding TMB serially may improve the signal-to-noise ratio of the reaction. Results illustrated in FIG. 212 show that the addition of multiple rounds of TMB (5×10 μL and 3×50 μL) improved the signal in comparison to the same volume but given as a single volume (1×50 μL and 1×150 μL). Considering relatively weak performance in the control reaction, additional experiments and optimization would be beneficial.

### Example 44: Immobilization Facilitated Positive Feedback Loop System for Amplifying Signals with Two Programmable Nucleases

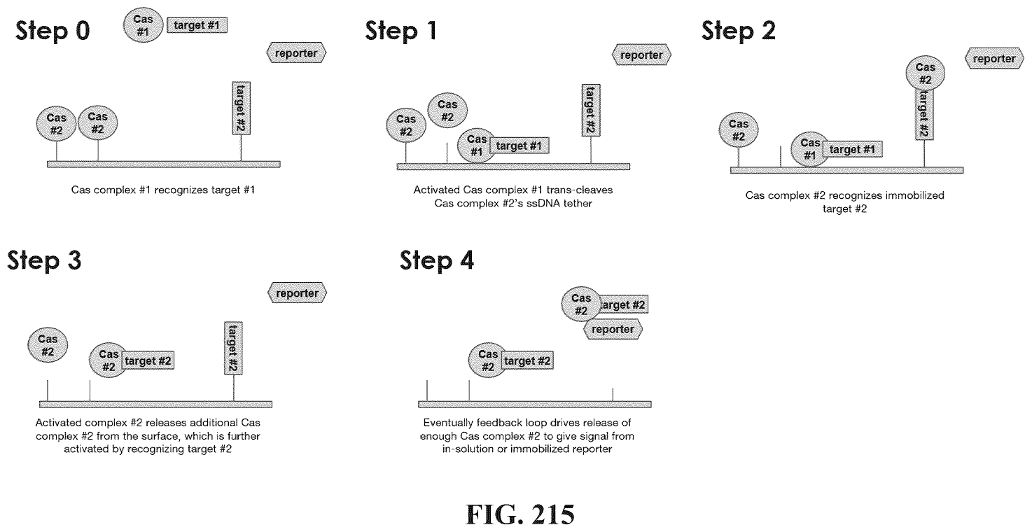

This example demonstrates a positive feedback loop system for amplifying the signal for each target nucleic acid molecule in a sample. The assay used in this example is illustrated diagrammatically in FIG. 214 and FIG. 215.

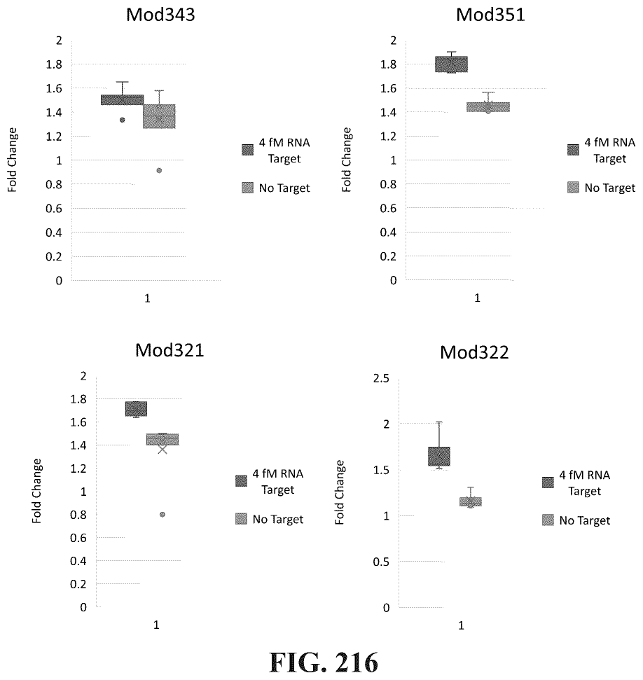

A mixture comprising a first programmable nuclease (21401, Cas #1) complexed with primary target-specific guide nucleic acids, a second programmable nuclease (21402, Cas #2) complexed with secondary target-specific guide nucleic acids immobilized to the surface, a secondary target nucleic acid (21403, target #2) immobilized to the surface, a reporter molecule in solution (21404), and finally, a primary target nucleic acid (21405, target #1) capable of interacting with the complex (21401, Cas #1) when introduced into solution. The programmable nuclease (21401, Cas #1) complex then trans-cleaves an ssDNA tether between the surface and the second programmable nuclease (21402, Cas #2) complex. This releases the second programmable nuclease (21402, Cas #2) into solution so it can recognize its corresponding target (21403, target #2) which activates the second programmable nuclease (21402, Cas #2) complex. The activation of the 21402 complex creates a feedback loop where further programmable nuclease 21402 can be released. Activated 21402 complex is also effective to transcollaterally cleave the reporter molecule. Cleavage of the immobilized nucleic acids separates the quencher moieties from the fluorescent moieties, thereby allowing the fluorescent moieties to produce a detectable fluorescent signal. In some embodiments, this assay can be conducted on hydrogels as described in Example 28. In some embodiments, this assay can be conducted using immobilized reporters as described in FIG. 216.

### Positive Feedback Loop Detection Assay for Low Copy Number Targets

Immobilized guide RNAs used in the assay to detect an immobilized secondary target (Target 2) were those shown in Table 13, with results shown in FIG. 216. Performance of the assay conditions were those described according to the following. A series of reactions were prepared with 7 spots each containing immobilized amine-modified dsDNA Target 2 (Mammuthus), ssDNA tether/guide RNA designed to hybridize to Target 2, and FAM-labeled reporter rep200 within N=1 NHS-coated wells. Four different ssDNA tether/guideRNA were tested-Mod343, Mod351, Mod321, or Mod322—as shown in TABLE 13. All cleavage reactions were carried out in DESPEC buffer in NHS-coated wells. Prior to initiating the detection assay, a Cas12 complexing reaction was prepared at a final concentration of 1× DESPEC buffer, 5 nM of a Cas12 variant (SEQ ID NO: 34), and 10 nM guide RNA R780 (targeting RNase P, “Target 1”) and incubated for 15 minutes at room temperature in solution to generate a first Cas complex as shown in FIG. 214 and FIG. 215 (21401, Cas #1). While the first Cas complex was being formed, uncomplexed Cas 12 variant was added to the prepared slide wells and allowed to complex with guideRNA immobilized to the slide to form an immobilized, second Cas complex as shown in FIG. 214 and FIG. 215 (21402, Cas #2). The first Cas complex (Cas #1, FIG. 214, 21401) was then added to slide wells along with 4 fM of RNaseP “Target 1” to initiate the detection assay and was incubated for 60 minutes at 55° C. on a thermomixer. Final fluorescence within the wells after 60 minutes was then imaged on a fluorescent microscope and analyzed for fold change in fluorescence versus fluorescence at time 0 (i.e., before the first Cas complex and first target were added to the wells). Reduced fluorescence (and a higher absolute fold change) indicated reporter cleavage and release of the FAM detection moiety from the surface of the well, with absolute fold change fluorescence results shown in FIG. 216 for each of the different immobilized guideRNAs tested.

TABLE 13
GuideRNAs for surface-immobilized target nucleic acid detection assays

| | |
|---|---|
| Mod343 | /5AmMC12/iSp18/iSp18/TTTTTTTTTTTTTTTTTTT/iSp18/iSp18/rUrArAr |
| | UrUrUrCrUrArCrUrArArGrUrGrUrArGrArUrGrCrCrGrArUrArArUrGrArU |
| | rGrUrArGrGrGrArU (SEQ ID NOS 1513 and 1485) |
| Mod351 | /5AmMC12/iSp18//TTTTTTTTTTTTTTTTTTT/iSp18/TArArUrUrUrCrUrArC |
| | rUrArArGrUrGrUrArGrArUrGrCrCrGrArUrArArUrGrArUrGrUrArGrGrGrAr |
| | U (SEQ ID NOS 1514 and 1486) |
| Mod321 | /5AmMC6/TTTTTTTTTTTTTTTTTTTTTTTTTTTTTTTTTTTTTTTTTTTTT |
| | AArUrUrUrCrUrArCrUrArArGrUrGrUrArGrArUrGrCrCrGrArUrArArUrGrAr |
| | UrGrUrArGrGrGrArU (SEQ ID NO: 1515) |
| Mod322 | /5AmMC6/TTTTTTTTTTTTTTTTTTTTrUrUrUrUTTTTTTTTTTTTTTTTTT |
| | TTTTTTTTAArUrUrUrCrUrArCrUrArArGrUrGrUrArGrArUrGrCrCrGrArUr |
| | ArArUrGrArUrGrUrArGrGrGrArU (SEQ ID NO: 1516) |

### Example 45: HRP-Bound gREP Spectrophotometric Assay for Detecting Target Nucleic Acids

This example describes production of a visible signal upon target nucleic acid detection, particularly in the absence of target nucleic acid amplification. In this experiment, reporters comprising HRP detection moieties linked to a tethered guide-reporter hybrid molecule (gREP) are immobilized to a surface by a linkage. The gREP forms a complex with a programmable nuclease, which is activated upon further complexing with a target nucleic acid. The activated programmable nuclease is effective to transcollaterally cleave one or more linkages connecting the HRP detection moiety to the remainder of the gREP. The HRP is released, and the supernatant containing the released HRP is then transferred to a secondary chamber containing an HRP substrate. When HRP is present in the supernatant it acts on the HRP substrate to produce a detectable signal. In some embodiments, the detectable signal can be detected by measuring an absorbance or fluorescence within the secondary chamber. A schematic representation of the assay is shown in FIG. 217.

### Example 46: Single-Step Nicking Enzyme Amplification Reaction (NEAR) with CRISPR-Based Detection of DNA and RNA (NECTR)

This example describes a single-step isothermal nicking enzyme amplification reaction (NEAR) for use with programmable nuclease-based detection reactions (e.g., DETECTR). The detection reaction incorporates CRISPR-based specificity with NEAR's rapid and robust amplification. A schematic of the NEAR amplification reaction can be found in FIG. 166, with particular attention being paid to primer and guide placement along with guide/primer overlap to aid in guide design.

LAMP, another isothermal amplification method, may take around 20-45 minutes of incubation time for sufficient amplification of a target sequence at a low copy number, which may be too long of a process for some point-of-care diagnostics settings. This duration can be mitigated through the incorporation of nicking enzyme assisted amplification reactions such as EXPAR, NEAR, and NEMA. In at least some instances, NEAR may be ideal for isothermal single-step reactions (e.g., HotPot reactions) due to its robust amplification speed at one temperature, though optimization may be beneficial in view of closely positioned primer pairs, which may be prone to off target activation. This example describes results showing development of NEAR for use with an illustrative Cas-based detection assay (e.g., DETECTR), which is ideal for amplifying and detecting targets of interest rapidly in a single-step reaction.

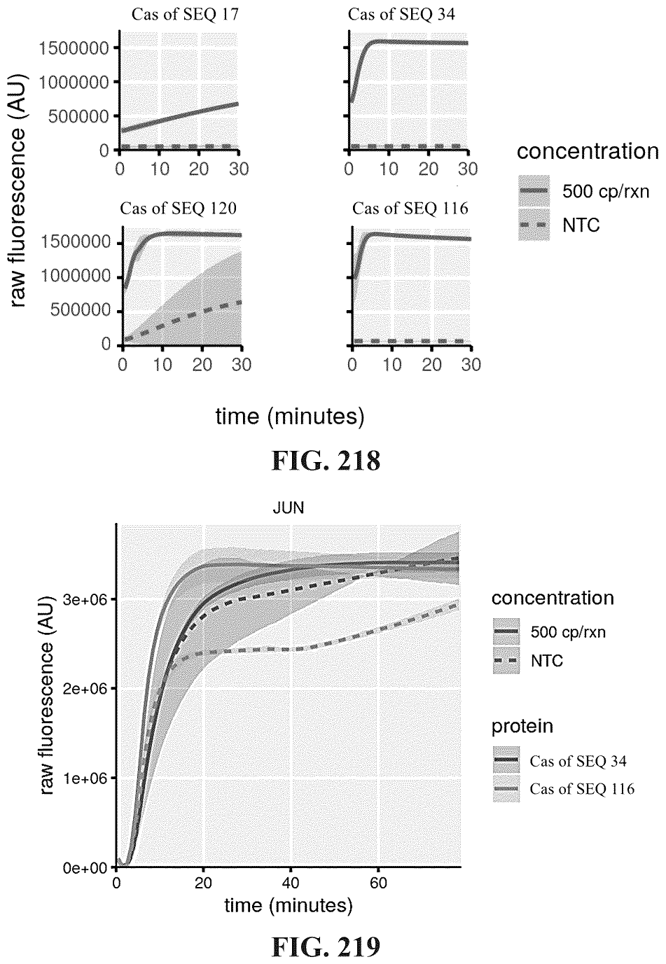

Cas variants were tested for performance in a high temperature and salt environment compatible with a single-step NEAR-detection reaction. Two-step NECTR reactions using a Cas 12 variant having SEQ ID NO: 34, along with two other thermostable variants (a Cas12 variant having SEQ ID NO: 120, and a Cas12 variant having SEQ ID NO: 116) were performed and showed high activity when run in NEAR buffer conditions (FIG. 218). Here, Cas complexing and DETECTR reactions were run in the NEAR reaction buffer (1× IsoAmp®+0.5×NEBuffer 3.1™). The Cas 12 variant, and other Cas variants (a Cas12 variant having SEQ ID NO: 120, and a Cas12 variant having SEQ ID NO:116), showed high activity when run in NEAR buffer conditions. Each Cas protein (40 nM final concentration) was pre-complexed with its guide nucleic acid (40 nM final concentration) at 37° C. for 30 mins in the NEAR reaction buffer. For each reaction, NEAR was performed on SARS-CoV-2 target RNA (500 copies/reaction, Twist, or 0 copies/reaction “NTC”) at 60° C. for 10 mins with Nt BstNBI nicking enzyme (0.3 U/ul final concentration), Bst 2.0 DNA polymerase (0.4 U/ul final concentration), Omniscript reverse transcriptase (0.2 U/ul final concentration), dNTPs (0.5 mM final concentration), forward primer (0.5 μM final concentration), and reverse primer (1 μM final concentration) as previously described, then 2 μL of the NEAR reaction was spiked into a solution containing one of the aforementioned Cas complexes and an FQ ssDNA reporter, and DETECTR was run for 30 mins at 37° C. and fluorescence signal indicative of reporter cleavage was monitored.

Next, one-step HotPot reactions were performed using the Cas 12 variant of SEQ ID NO: 34 and a Cas12 variant having SEQ ID NO:116 (FIG. 219), in which both proteins in unexpectedly showed low signal-to-noise in the combined reaction. The Cas enzymes were pre-complexed with their guide RNAs at 37° C. for 30 mins as described herein before being added directly into the NEAR reaction mix described herein (e.g., with reference to FIG. 218) containing an FQ ssDNA reporter. The one-pot NECTR reaction was incubated for 120 mins at 60° C. and fluorescence signal indicative of reporter cleavage was monitored. This resulted in low signal-to-noise ratios, with significant signal being observed in the NTC (“no template control”) conditions, suggesting that something other than the target was turning on the NECTR reaction. It was hypothesized that the guide RNA may have been used as a template for the primers, thus triggering the NECTR reaction to proceed even when no target was added.

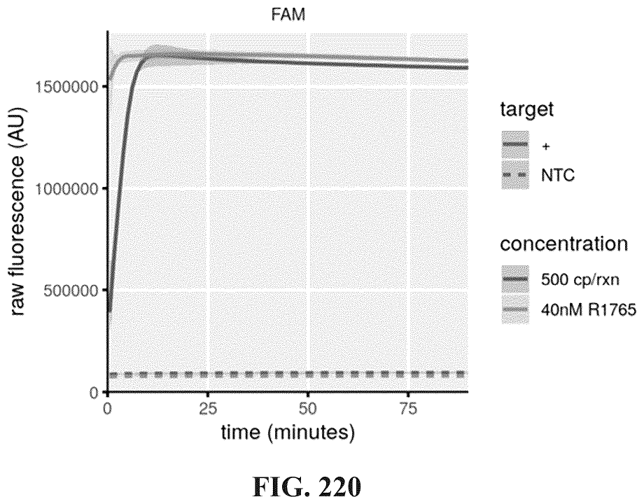

A NEAR reaction was thus performed using either the normal SARS-CoV-2 RNA template or the guide RNA as a template, with all other conditions the same as described previously with respect to FIG. 218 (FIG. 220). Briefly, the Cas12 variant having SEQ ID NO: 34 was pre-complexed with its guide RNA at 37° C. for 30 mins in the NEAR reaction buffer. For each reaction, NEAR was run at 60° C. for 10 mins (with 500 copies/reaction SARS-CoV-2 target RNA or 40 nM guide RNA being added as the template), then 2 μL of the NEAR reaction product was spiked into a solution containing the Cas complex and an FQ ssDNA reporter The DETECTR reaction was the run for 120 mins at 60° C. and fluorescence signal indicative of reporter cleavage was monitored. The reaction proceeded by the guide just as well as the template under the conditions tested, with both conditions having robust fluorescence, indicating off target amplification of the guide itself was occurring.

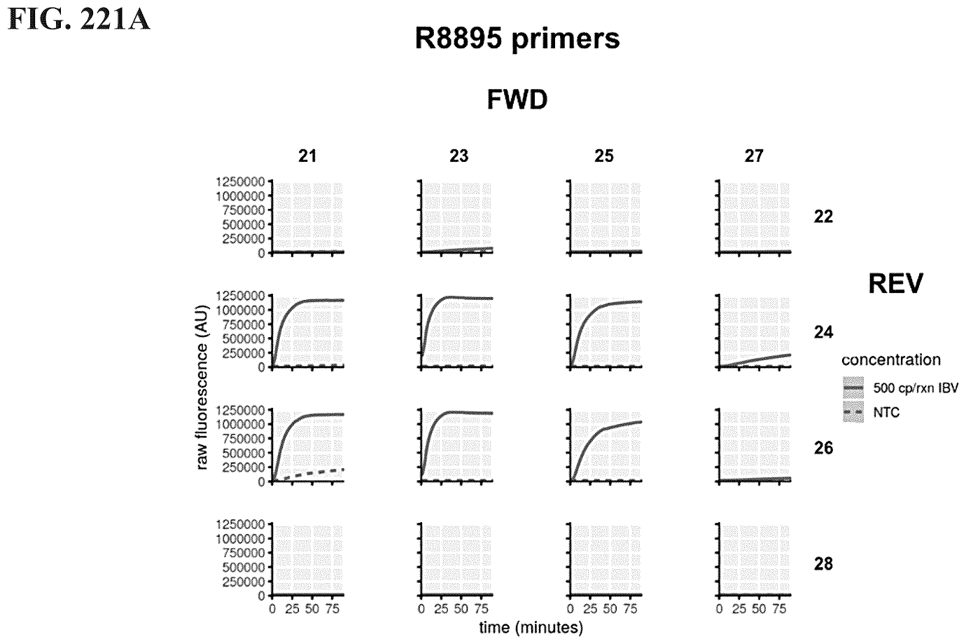

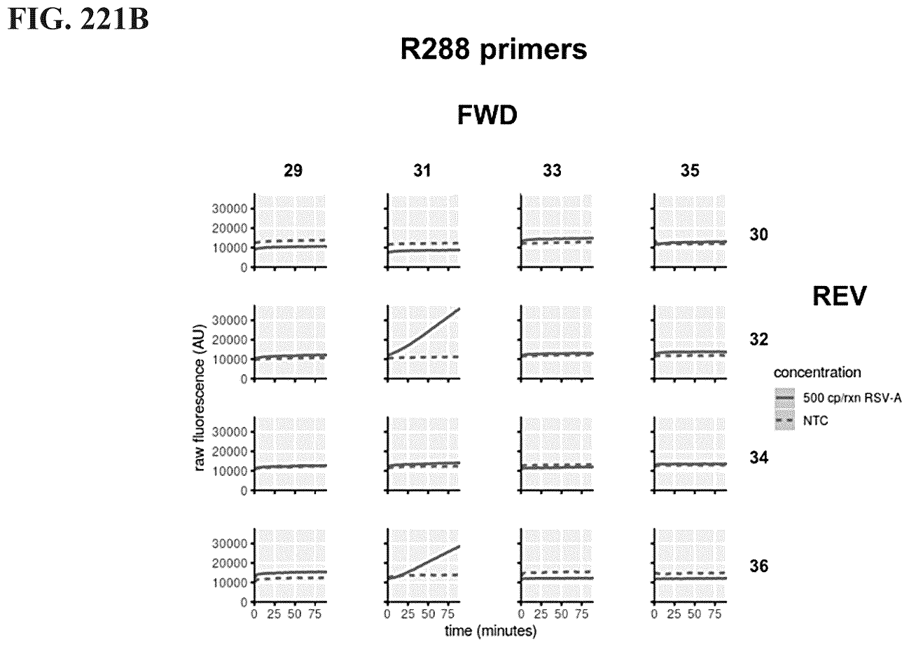

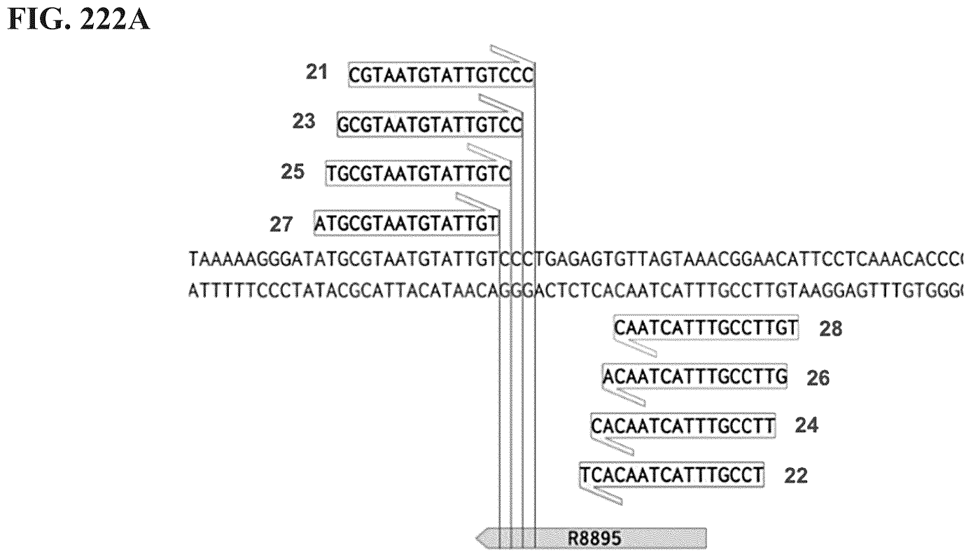

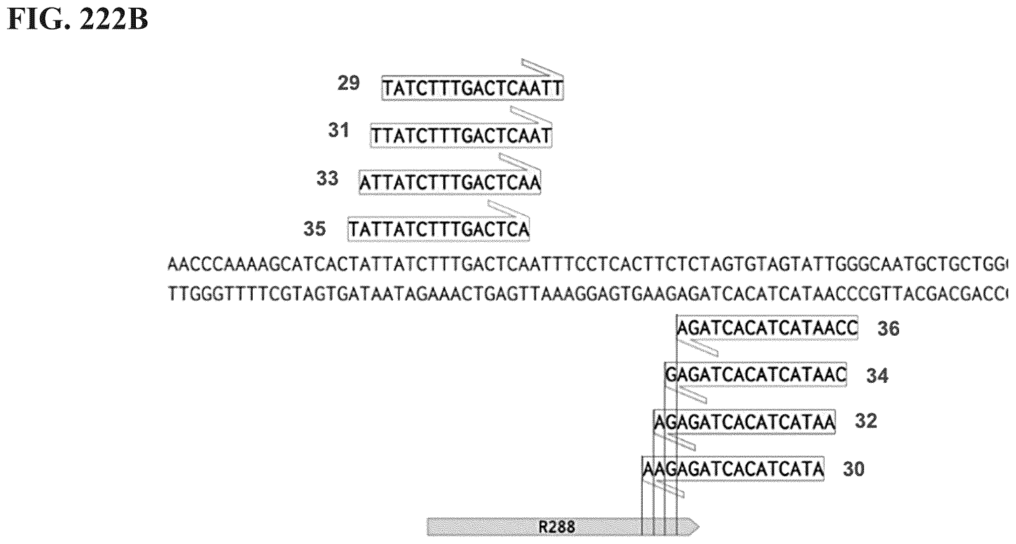

Given that the guide RNA itself could be used to trigger the NEAR reaction, new primer sets were designed around two alternative target sites, R8895 for influenza B virus (IBV) (FIGS. 221A, 222A) and R288 for RSV-A (FIGS. 221B, 222B). The primer sets were optimized for reduced overlap with the guide RNA while still maintaining the desired amplification speed of NEAR through single base pair shifts slightly increasing the length of the amplified region, (FIGS. 222A-222B graphically illustrate the guide/primer overlaps for IBV and RSV-A, respectively). The Cas12 variant of SEQ ID NO: 34 was pre-complexed with its guide RNA (R8895 for IBV or R288 for RSV-A) at 37° C. for 30 mins in NEAR reaction buffer as described herein. For each reaction, NEAR was run at 60° C. for 10 mins with different primer pairs (IBV: forward primers 21, 23, 25, or 27 in combination with reverse primers 22, 24, 26, or 28; RSV-A: forward primers 29, 31, 33, or 35 in combination with reverse primers 30, 32, 34, or 36) and with 500 copies/reaction or 0 copies/reaction (“NTC”) of IBV target RNA or RSV-A target RNA as described herein 2 μL of NEAR reaction product was then spiked into a DETECTR reaction with the Cas12 variant complex and an FQ-ssDNA reporter and incubated for 120 mins at 60° C. while fluorescence signal indicative of reporter cleavage was monitored as described herein.

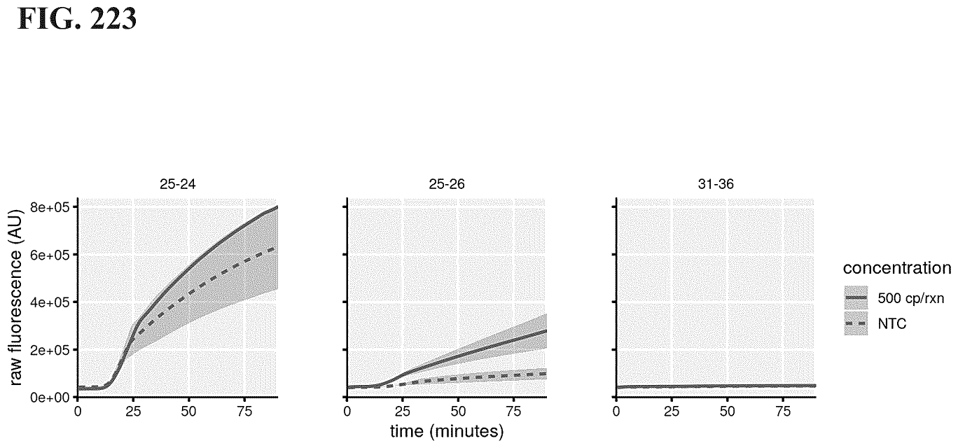

Three primer sets (25-24 and 25-26 for IBV, and 31-36 for RSV-A) were chosen for testing in the single-step based NECTR reaction due to their exhibiting high signal-to-noise (as demonstrated in FIGS. 221A-221B) with little guide overlap (as shown in FIGS. 222A-222B). Because NEAR is optimal with a small amplified region, designing primers to not overlap the guide RNA, while still keeping this region small enough for NEAR, can be difficult. Nonetheless, as shown in FIG. 223, the primer set of 25-26 resulted in high signal-to-noise in the HotPot NECTR reaction (which was performed as described with respect to FIG. 219).

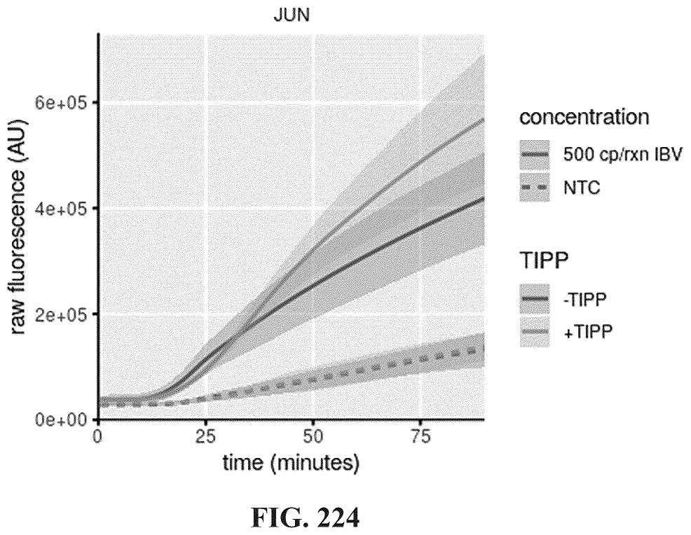

These reactions were next replicated with the addition of TIPP or in the absence of TIPP (FIG. 224). TIPP (thermostable inorganic pyrophosphatase) has previously been shown to help with Mg2+ concentration and enhancing separation between +/−targets (see, e.g., FIG. 164). The results with primer set 25-26 were successfully replicated and it was found that TIPP somewhat improved signal-to-noise over time.

### Example 47: Sample Preparation with Savinase® (Aka Subtilisin 309) to Remove Nucleases Having Endogenous RNase Activity Prior to DETECTR

This example describes a method of lysing a sample with a protease to release nucleic acids so that they are accessible to a programmable nuclease (e.g., in a DETECTR reaction). This example describes the use of Savinase® (Subtilisin 309) in a universal prep buffer for removing nucleases and thus reducing endogenous RNase activity. Biological samples, including saliva, nasal fluid, sputum, and blood, typically contain endogenous RNase species. Programmable nuclease-based detection assays for RNA viruses on crude sample preps often fail due to the presence of these RNases, which can cause degradation of the naked RNA viral template when it is released from viral particles during sample preparation. As RNases are protein in nature, the use of proteases can remove them, however the high variety of different RNase species in a sample means that few proteases will be able to remove all RNases present in the sample. It may also be beneficial to inhibit native RNase activity, particularly when the sample will be used in a programmable nuclease-based detection reaction, in order to prevent degradation of added nucleases (e.g., CRISPR-Cas enzymes) by the native RNAses. However, such nucleases may themselves be targets of proteases, therefore any protease used for sample preparation should preferably be removed, denatured, or otherwise inactivated before the sample is exposed to programmable nuclease-based detection reagents and conditions (e.g., DETECTR reaction compositions).

In this example, Savinase®, a serine protease, was tested for activity against the RNases present in fresh saliva samples using an RNase Reporter Assay (described below), and resulted in significantly reduced RNase activity. Savinase® was shown to be inhibited by 10 mM PMSF (phenylmethylsulfonyl fluoride, protease inhibitor). 10 mM PMSF was found to have no significant inhibition of a DETECTR reaction run using CasM.26. De-salted Savinase® successfully reduced saliva RNase levels, so that after treatment, following the addition of PMSF and spike-in of SARS-CoV-2 synthetic RNA, DETECTR was able to produce a positive signal. Without Savinase® treatment, RNase activity in saliva resulted in DETECTR failure.

Savinase® was purchased from Sigma® (P3111). Savinase® was initially screened alongside other proteases for activity against RNases from fresh saliva (50% v/v) in 10 mM Tris pH8. Saliva was chosen as the test sample. An RNase reporter assay (0.01% v/v Rep132 in 10 mM Tris pH8, incubated at 37° C. for 1 hour on QS5 using Cy5 channel for detection every 25s) was used to test the RNase activity following incubation at 37° C., 55° C., or 65° C. with 5% protease (v/v) for 1 hour. Savinase® showed the lowest RNase activity, with the preferred incubation temperature being 65° C. This was then repeated using 1× MB1 (buffer), 5% v/v protease, incubation at 37° C., 55° C., or 65° C. for 1 hour, and RNase activity measured using the FQ RNA reporter Rep132 (5′-Alex647N/rUrUrUrUrU/IAbRQSp-3′) in MB1. Again, Savinase® showed the lowest RNase activity, with the preferred incubation temperature tested being 65° C.

Savinase® was then tested against the other proteases which demonstrated some RNase removal at comparable activity concentrations of 3U, where possible. The lot of Savinase® used was documented as having 18U/g and the density of Savinase® at 1.16 g/mL, indicating an activity of 0.02U/uL. 30 μL of Savinase® (corresponding to 0.63U) was used in a 100 μL reaction (50 μL saliva, 20 μL 5× MB1), at 37° C., 55° C., or 65° C. for 1 hour; followed by RNase Reporter Assay (described above). Despite the units of Savinase® used being ˜5-fold less than for many of the other enzymes tested, Savinase® still showed to clearly have the best RNase removal of the proteases tested. The protease inhibitor PMSF was then tested for activity against Savinase® following the incubation at 55° C. and 65° C. for 1 hour; at 0, 0.1 mM, 1 mM, and 10 mM in 50% isopropanol, at room temperature (no incubation time) and Pierce™ Protease Activity assay (Thermo Scientific® 23266), 25° C. for 45 m on QS5 with signal detected using FAM channel, measuring every 25s. 10 mM PMSF was shown to significantly reduce Savinase® activity.

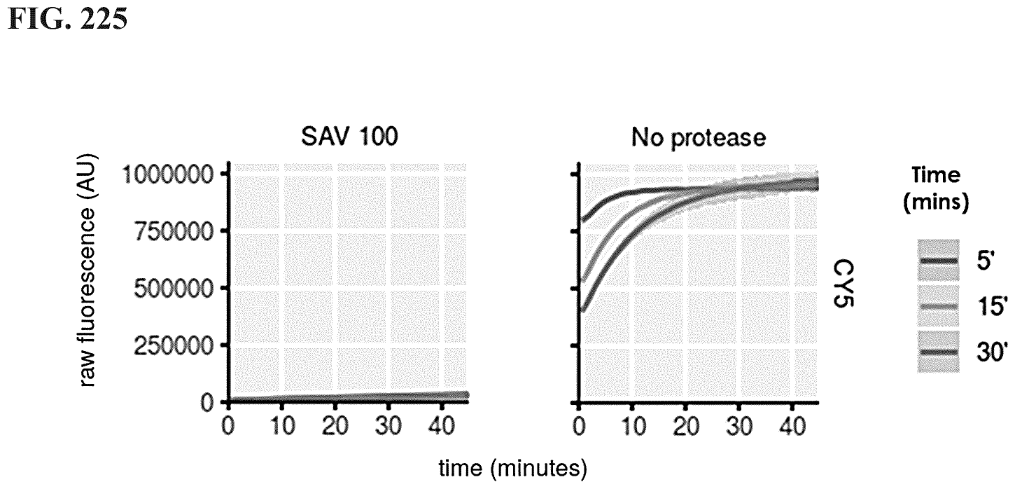

Further testing of Savinase® alone and in combination with the next most RNase-removing protease (Protamex, Sigma® P0029) was carried out using different ratios of each enzyme combined. Savinase® was confirmed to have significant anti-RNase activity via the RNAse reporter assay described above (FIG. 225). 30 μL of Savinase® (corresponding to 0.63U) was used in a 100 μL reactions (50 μL saliva, 20 μL 5× MB1) for 5, 15, or 30 minutes at 65° C. to determine whether RNAse activity was reduced with treatment with Savinase®. The RNase reporter assay (0.01% v/v Rep132 in 10 mM Tris pH8, incubated at 37° C. for 1 hour on QS5 using Cy5 channel for detection every 25s) was used to test the RNase activity of Savinase®. 100% Savinase was shown to have superior anti-RNase activity over Protamex® via the RNAse reporter assay.

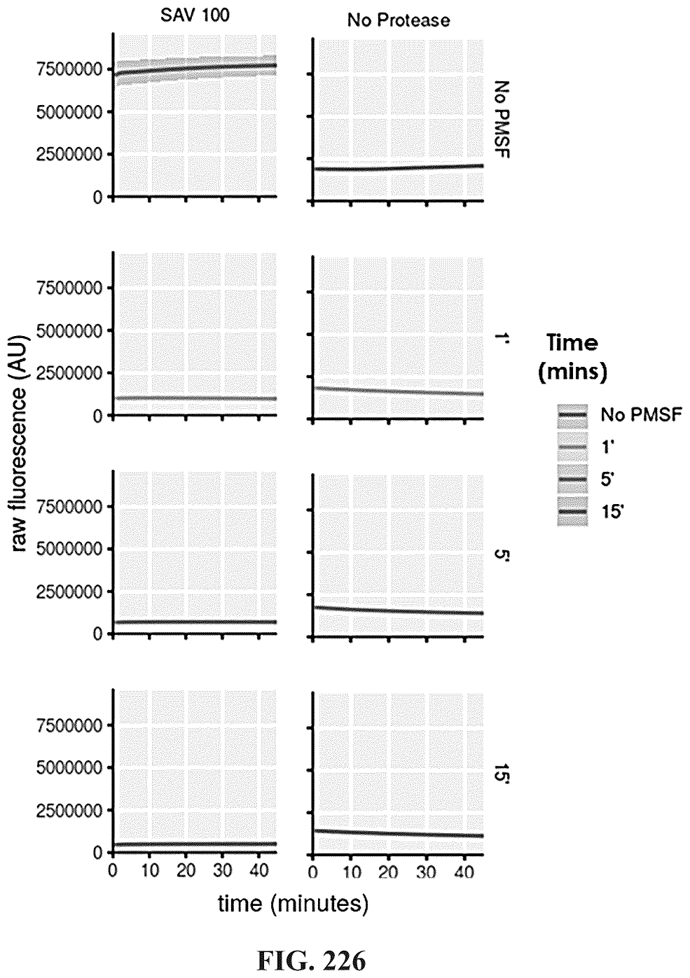

Savinase® also showed significant reduction in Protease Activity Assay following inhibition with 10 mM PMSF (in 15% isopropanol) after just 1 minute at room temperature; this was reduced further with longer incubation times (5 minutes and 15 minutes) (FIG. 226). The RNAse reporter assay was run as before following treatment of Savinase® with PMSF for the stated time periods. Other protease inhibitors were tested for inhibition of Savinase®, initially at the suggested working concentrations and then at 10× working concentrations, as the working concentration for PMSF is 1 mM, but 10 mM showed increased activity against Savinase®.

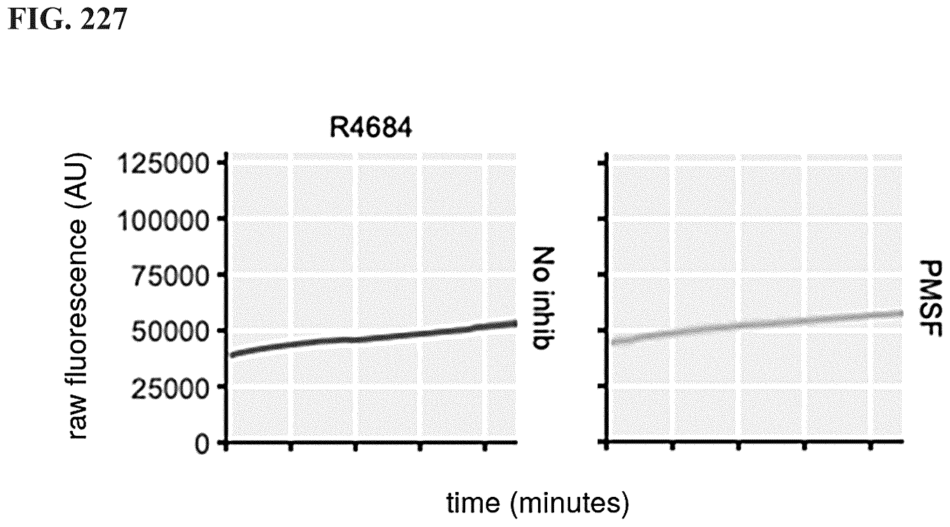

Protease inhibitors (including PMSF) were tested for activity against CasM.26 via a DETECTR reaction with 1.5M cp/rxn Twist SARS-CoV-2 RNA, R4684 guide RNA (5′-GCCACCCCAAAAAUGAAGGGGACUAAAACAAGAAGAAUUCAGAUUUUUAA-3′ (SEQ ID NO: 1517)), (62.5 nM final concentration), reporter Rep132 (100 nM final concentration), and buffer MB1. PMSF was shown not to adversely affect DETECTR (FIG. 227).

Some commercially-purchased Savinase® was found to be non-compatible with DETECTR, unless in very dilute concentrations. It was hypothesized that salts in the commercially-purchased Savinase® may have a deleterious effect on the DETCTR reaction. To test this, commercially-purchased Savinase® was subjected to desalting to determine whether salt concentration may contribute to reduced activity of the DETECTR reaction. Desalting of Savinase® on PD10 columns was shown to remove calcium from the Savinase® via an Alginate Polymerization Assay.

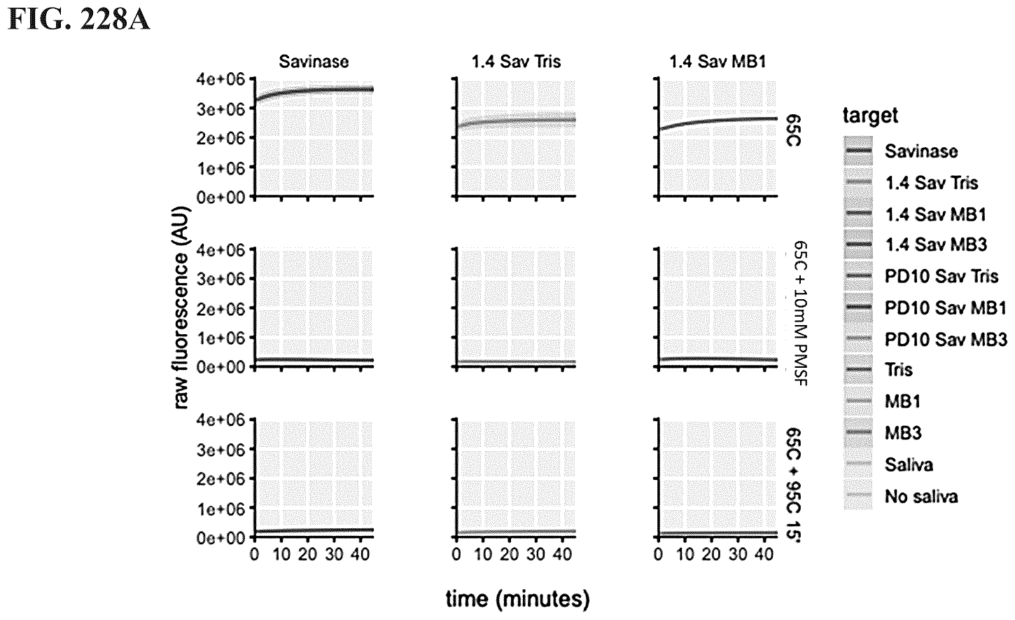

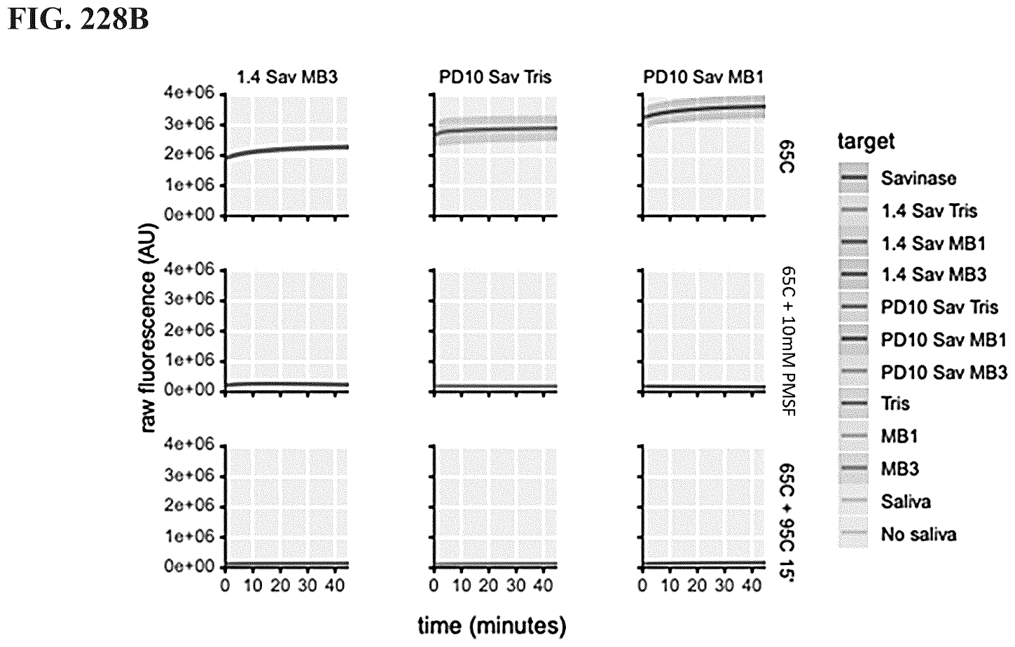

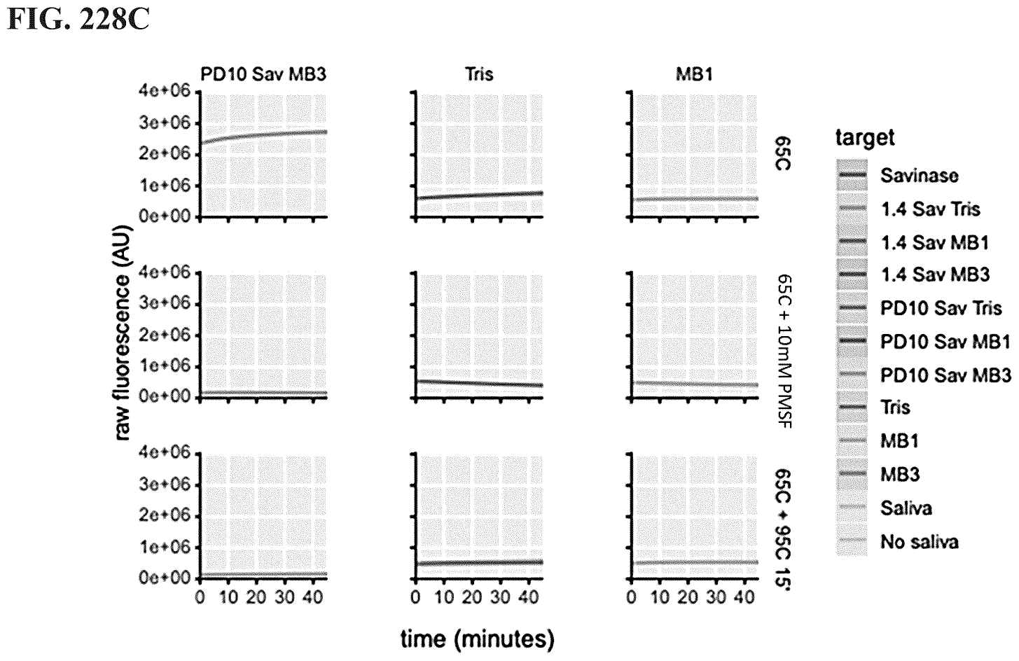

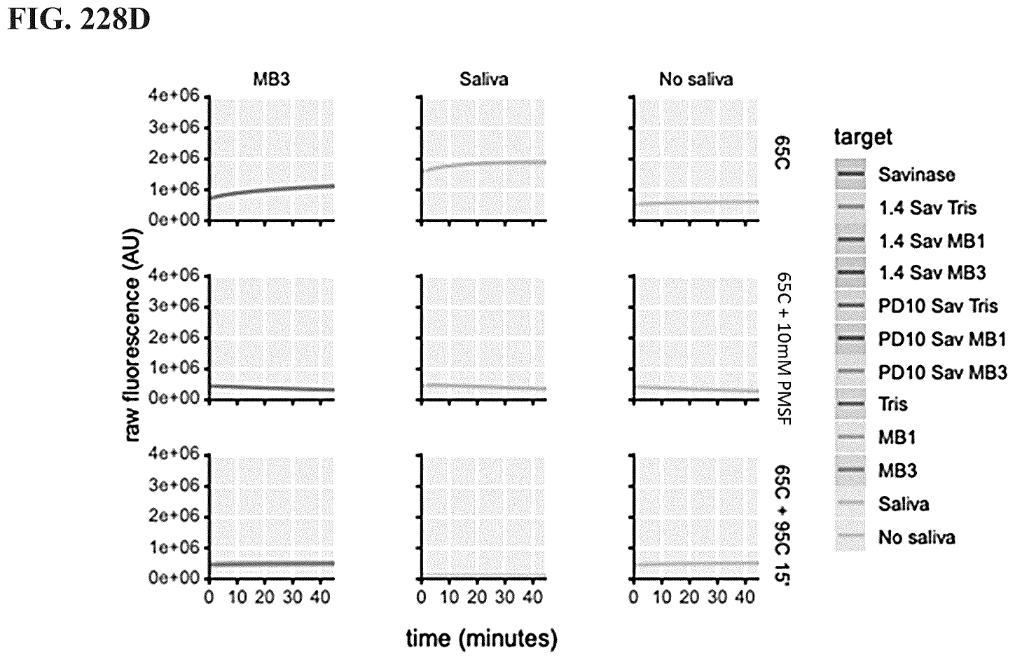

FIGS. 228A-228D depict a protease assay comparing commercially supplied Savinase®, 1.4× diluted Savinase® with PD10 cleaned Savinase® eluted in Tris, MB1 or MB3, & different elution buffers alone, all with 50% saliva (other than the No Saliva control), and the Saliva control has no Savinase®. Results show the endogenous protease activity in Saliva. All samples were heated at 65° C. for 5 m followed by no more treatment, the addition of 10 mM PMSF or 95° C. for 15m. Pierce™ Protease Activity assay was used to define protease activity (measured on QS5 at 25° C. for 90 cycles if 25 seconds detection, 5 seconds). De-salted Savinase® retained its proteolytic activity. Both PMSF and heat treatment were sufficient to reduce protease activity of all Savinase® species (as-purchased or following de-salting).

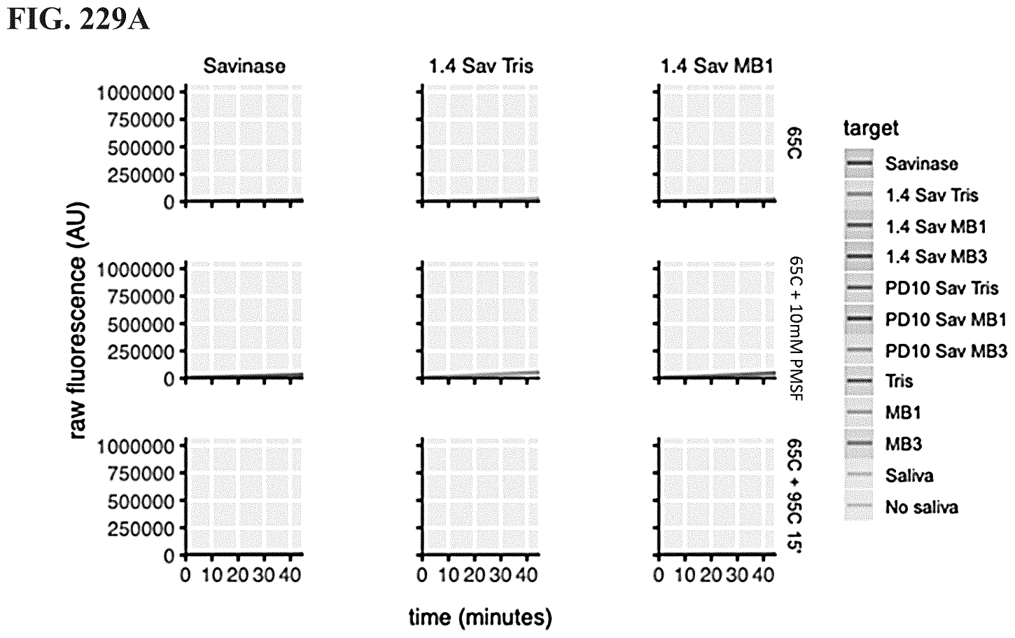

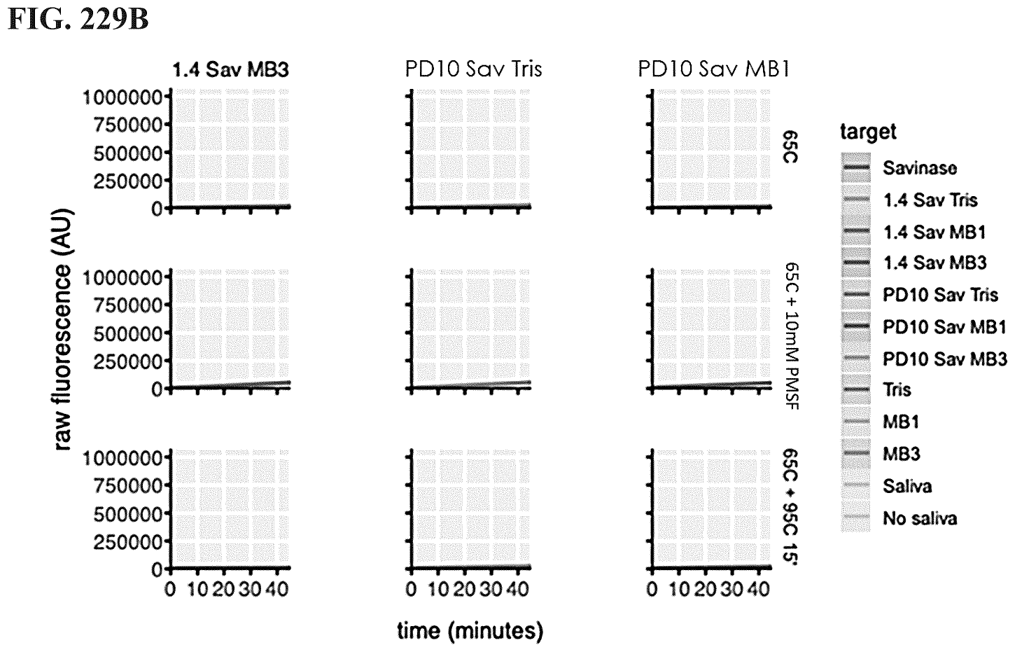

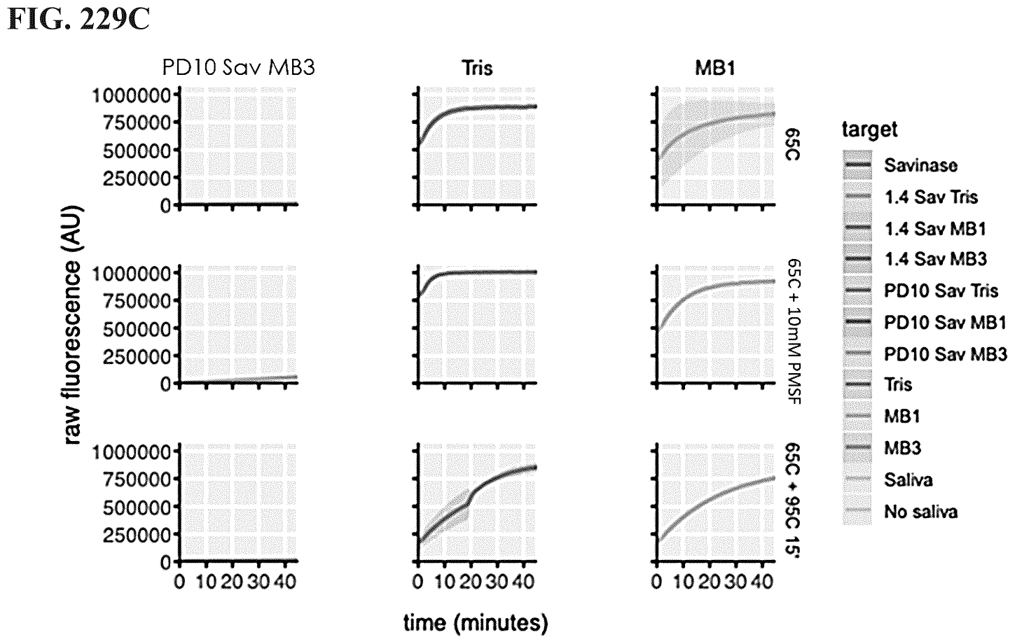

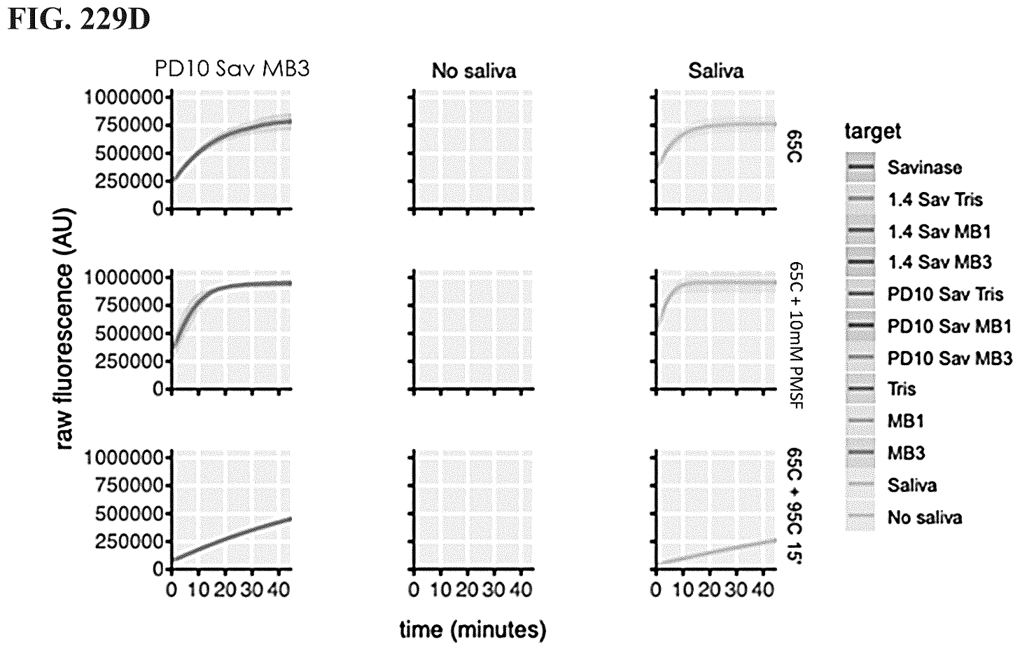

FIGS. 229A-229D depict the remaining RNase activity following treatment with commercially supplied Savinase®, 1.4× diluted Savinase®, or PD10 cleaned Savinase® eluted in Tris, MB1 or MB3, & different elution buffers alone, all with 50% saliva (other than the No Saliva control), and the Saliva control has no Savinase®. Results show the endogenous protease activity in Saliva. All samples were heated at 65 C for 5 m followed by no more treatment, the addition of 10 mM PMSF or 95° C. for 15m. RNase Reporter assay (Rep132 in MB1) was used to define protease activity (measured on QS5 at 37° C. for 90 cycles if 25s detection, 5s). Desalted Savinase® was able to remove native RNase activity from saliva.

Desalting resulted in a Savinase® solution which still retained protease activity, was still highly effective at removing RNase activity from saliva, and showed potential compatibility with RNA-based DETECTR. In an exemplary DETECTR reaction, saliva treated with PD10 desalted Savinase® is incubated at 65° C. for 5 minutes, denatured with 10 mM PMSF at room temperatures, spiked with 2M cp/rxn SARS-CoV-2 synthetic RNA (Twist), and then incubated with CasM.26, R4684 guideRNA, reporter Rep132, and MB1 buffer at 37° C. Fluorescence is monitored over time to determine whether reporter is cleaved by the Cas enzyme as described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein can be employed. It is intended that the following claims define the scope of the invention and that methods and structures within the scope of these claims and their equivalents be covered thereby.

1. A composition for detecting a target nucleic acid, the composition comprising a programmable nuclease, a guide nucleic acid, a first reporter, an enzyme, and a second reporter, wherein:
(a) the guide nucleic acid is configured to bind to the target nucleic acid;
(b) the programmable nuclease is effective to cleave the first reporter in response to formation of a complex comprising the programmable nuclease, the guide nucleic acid, and the target nucleic acid;
(c) the cleavage of the first reporter is effective to separate a first nucleic acid section from a second nucleic acid section thereof;
(d) the first nucleic acid section is effective to activate the enzyme; and
(e) the activated enzyme is effective to cleave the second reporter to produce a detectable product comprising a detection moiety.

2. The composition of claim 1, wherein the enzyme is an endonuclease and the second reporter comprises a polynucleotide substrate of the enzyme.

3. The composition of claim 2, wherein the endonuclease is a NucC endonuclease.

4. The composition of claim 3, wherein the first nucleic acid section comprises adenosine residues.

5. The composition of claim 4, wherein the adenosine residues comprise cyclic adenylate (cA3).

6. The composition of claim 1, wherein the second nucleic acid section comprises RNA residues, optionally wherein the RNA residues comprise a plurality of uracil residues.

7. The composition of claim 1, wherein the second nucleic acid section comprises DNA residues, optionally wherein the DNA residues comprise a plurality of thymine residues.

8. The composition of claim 1, wherein (a) the second reporter comprises a fluorescent label and a quencher, and (b) cleavage of the second reporter by the activated enzyme is effective to separate the fluorescent label from the quencher.

9. A method of detecting a target nucleic acid in a sample, the method comprising:
(a) contacting the sample with the composition of claim 1;
(b) cleaving the first reporter with the programmable nuclease in response to formation of the complex comprising the programmable nuclease, the guide nucleic acid, and the target nucleic acid, thereby releasing the first nucleic acid section;
(c) activating the enzyme with the first nucleic acid section;
(d) cleaving the second reporter with the activated enzyme, thereby producing the detectable product comprising the detection moiety; and
(e) detecting the detection moiety, thereby detecting the presence of the target nucleic acid in the sample.

10. The method of claim 9, wherein (a) the second reporter comprises a polynucleotide substrate of the enzyme, and (b) the enzyme is a NucC.

11. The method of claim 10, wherein step (d) is performed at a temperature of at least 40° C.

12. A device for detecting a target nucleic acid, the device comprising:
a sample interface configured to receive a sample;
a first sample chamber in fluid communication with the sample interface and configured to hold a first predetermined volume of the sample;
a first reaction chamber in fluid communication with the first sample chamber and comprising a first set of amplification reagents and a first set of detection reagents, wherein the first set of amplification reagents are configured to amplify a first target nucleic acid, wherein the first set of detection reagents comprises a first programmable nuclease, a first guide nucleic acid configured to bind to the first target nucleic acid, and a first reporter configured to release a first detection moiety when cleaved by the first programmable nuclease following binding of the first guide nucleic acid to the first target nucleic acid;
a first valve disposed between the first sample chamber and the first reaction chamber and configured to regulate flow therebetween;
a first lateral flow assay strip configured to be inserted into the first reaction chamber and capture the first detection moiety when released and thereby detect a presence or absence of the first target nucleic acid in the sample;
a second sample chamber in fluid communication with the sample interface and configured to hold a second predetermined volume of the sample;
a second reaction chamber in fluid communication with the second sample chamber and comprising a second set of amplification reagents and a second set of detection reagents, wherein the second set of amplification reagents are configured to amplify a second target nucleic acid, wherein the second set of detection reagents comprises a second programmable nuclease, a second guide nucleic acid configured to bind to the second target nucleic acid, and a second reporter configured to release a second detection moiety when cleaved by the second programmable nuclease following binding of the second guide nucleic acid to the second target nucleic acid;
a second valve disposed between the second sample chamber and second first reaction chamber and configured to regulate flow therebetween; and
a second lateral flow assay strip configured to be inserted into the second reaction chamber and capture the second detection moiety when released and thereby detect a presence or absence of the second target nucleic acid in the sample,
wherein the first and second reaction chambers are fluidly independent of one another.

13. A method for detecting a plurality of target nucleic acids, the method comprising:
applying the sample to the sample interface of the device of claim 12;
transferring the first predetermined volume of the sample into the first sample chamber;
transferring the second predetermined volume of the sample into the second sample chamber;
transferring the first predetermined volume from the first sample chamber to the first reaction chamber;
transferring the second predetermined volume from the second sample chamber to the second reaction chamber;
amplifying the first target nucleic acid in the first reaction chamber;
amplifying the second target nucleic acid in the second reaction chamber;
binding the first target nucleic acid with the first guide nucleic acid, thereby cleaving the first detection moiety from the first reporter in the first reaction chamber;
binding the second target nucleic acid with the second guide nucleic acid, thereby cleaving the second detection moiety from the second reporter in the second reaction chamber;
inserting the first lateral flow assay strip into the first reaction chamber;
inserting the second lateral flow assay strip into the second reaction chamber;
capturing the first detection moiety with the first lateral flow assay strip, thereby detecting the presence of the first target nucleic acid; and
capturing the second detection moiety with the second lateral flow assay strip, thereby detecting the presence of the second target nucleic acid.

14. A device for detecting a target nucleic acid, the device comprising:
a sample interface configured to receive a sample;
a first reaction chamber in fluid communication with the sample interface and comprising a first set of amplification reagents and a first set of detection reagents, wherein the first set of amplification reagents are configured to amplify a first target nucleic acid, wherein the first set of detection reagents comprises a first programmable nuclease, a first guide nucleic acid configured to bind to the first target nucleic acid, and a first reporter configured to release a first detection moiety when cleaved by the first programmable nuclease following binding of the first guide nucleic acid to the first target nucleic acid;
a first lateral flow assay strip in fluid communication with the first reaction chamber and configured to capture the first detection moiety when released and thereby detect a presence or absence of the first target nucleic acid in the sample;
a second reaction chamber in fluid communication with the sample interface and comprising a second set of amplification reagents and a second set of detection reagents, wherein the second set of amplification reagents are configured to amplify a second target nucleic acid, wherein the second set of detection reagents comprises a second programmable nuclease, a second guide nucleic acid configured to bind to the second target nucleic acid, and a second reporter configured to release a second detection moiety when cleaved by the second programmable nuclease following binding of the second guide nucleic acid to the second target nucleic acid; and
a second lateral flow assay strip in fluid communication with the second reaction chamber and configured to capture the second detection moiety when released and thereby detect a presence or absence of the second target nucleic acid in the sample,
wherein the first and second reaction chambers are fluidly independent of one another.

15. (canceled)

16. (canceled)

17. A device for detecting a target nucleic acid, comprising:
a sample interface configured to receive a sample;
a reaction chamber in fluid communication with the sample interface, wherein the reaction chamber comprises a plurality of reporters and thermostable inorganic pyrophosphatase;
a programmable nuclease and a guide nucleic acid disposed within the reaction chamber, the guide nucleic acid being configured to bind to a target nucleic acid;
wherein each of the plurality of reporters is configured to release a detection moiety when cleaved by the programmable nuclease following binding of the guide nucleic acid to the target nucleic acid, and
wherein release of the detection moiety is indicative of a presence or absence of the target nucleic acid.

18. A device for detecting a target nucleic acid, comprising:
a sample interface configured to receive a sample;
a first reaction chamber in fluid communication with the sample interface, wherein the first reaction chamber comprises amplification reagents configured to amplify the sample, said amplification reagents comprising a forward primer, a reverse primer, dNTPs, a DNA polymerase, and a nicking endonuclease;
a second reaction chamber in fluid communication with the first reaction chamber, wherein the second reaction chamber comprises a plurality of reporters;
a programmable nuclease and a guide nucleic acid disposed within the second reaction chamber, the guide nucleic acid being configured to bind to a target nucleic acid;
wherein each of the plurality of reporters is configured to release a detection moiety when cleaved by the programmable nuclease following binding of the guide nucleic acid to the target nucleic acid, and
wherein release of the detection moiety is indicative of a presence or absence of the target nucleic acid in the sample.

19.-71. (canceled)

72. A composition for detecting a target nucleic acid in a reaction chamber, the composition comprising a programmable nuclease, a guide nucleic acid, a forward primer, a reverse primer, a polymerase, a nicking endonuclease, and a reporter, wherein:
(a) the guide nucleic acid is configured to bind to the target nucleic acid;
(b) the forward primer comprises (i) a 5′ portion comprising a first hairpin, and (ii) a 3′ portion that is configured to bind the target nucleic acid at a first overlapping region with respect to the guide nucleic acid;
(c) the reverse primer comprises (i) a 5′ portion comprising a second hairpin, and (ii) a 3′ portion that is configured to bind a complement of the target nucleic acid at a second overlapping region with respect to the guide nucleic acid;
(d) the first and second hairpins are cleavage substrates for the nicking endonuclease;
(e) the programmable nuclease is effective to cleave the reporter in response to formation of a complex comprising the programmable nuclease, the guide nucleic acid, and (i) the target nucleic acid or (ii) an amplicon of the target nucleic acid; and
(f) the cleavage of the reporter is effective to produce a detectable product comprising a detection moiety.

73. The composition of claim 72, wherein (a) the sequence of the target nucleic acid to which the 3′ portion of the first primer is configured to bind defines a first sequence of the target nucleic acid; (b) the sequence of the 3′ portion of the reverse primer defines a second sequence of the target nucleic acid; and (c) the first sequence and second sequence are separated by about 5 to about 10 nucleotides along the target nucleic acid.

74. The composition of claim 72, wherein the 3′ portions of the forward primer and reverse primer are about 16 to about 20 nucleotides in length.

75. The composition of claim 72, wherein overlap between the 3′ portion of the reverse primer and the sequence to which the guide nucleic acid is configured to bind overlap by 1 to 5 nucleotides, 2 to 5 nucleotides, or 3 nucleotides.

76. The composition of claim 72, wherein the first hairpin and/or the second hairpin are 10 to 20 nucleotides in length, 16 to 20 nucleotides in length, or 16 nucleotides in length.

77. The composition of claim 72, wherein the programmable nuclease is a Cas protein, optionally wherein the Cas protein is a Cas 12 protein or a Cas14 protein.

78. A method of detecting a target nucleic acid in a sample, the method comprising:
(a) contacting the sample with the composition of claim 72;
(b) performing nicking enzyme amplification reaction (NEAR) reaction to amplify the target nucleic acid;
(c) forming a complex comprising the programmable nuclease, the guide nucleic acid, and (i) the target nucleic acid, or (ii) an amplicon of the target nucleic acid;
(d) cleaving the reporter with the programmable nuclease activated by formation of the complex, thereby producing the detectable cleavage product; and
(e) detecting the detection moiety, thereby detecting the presence of the target nucleic acid in the sample.

79. The method of claim 78, wherein steps (b) through (d) are performed at about the same temperature.
